(19) United States
(12) Patent Application Publication
(10) Pub. No.: US 2023/0250096 A1
(43) Pub. Date: Aug. 10, 2023

(54) SUBSTITUTED PHENYL-1H-PYRROLO[2,3-c] PYRIDINE DERIVATIVES

(71) Applicant: Janssen Pharmaceutica NV, Beerse (BE)

(72) Inventors: Wei Cai, Shanghai (CN); Johannes Wilhelmus J. Thuring, Antwerpen (BE); Fabian Hulpia, Sint-Niklaas (BE); Xuedong Dai, Shanghai (CN); Ming Li, Shanghai (CN); Xiangjun Deng, Shanghai (CN); Chao Liang, Shanghai (CN); Alicia Tee Fuay Ng, Franklin Park, NJ (US); Zhen Sun, Shanghai (CN); Zhigao Zhang, Shanghai (CN); Samuel Dominique Demin, Mechelen (BE); Natalia Nikolaevna Dyubankova, Heverlee (BE); Matthiew Dominique Jouffroy, Berchem (BE); Susan Lepri, Antwerpen (BE); Nicolas Freddy J. Darville, Linkhout (BE); Vineet Pande, Vosselaar (BE); Wim Bert Griet Schepens, Sint-katelijne-Waver (BE); James Patrick Edwards, Ambler, PA (US); Olivier Alexis Georges Querolle, Saint Vigor (FR)

(21) Appl. No.: 18/015,661

(22) PCT Filed: May 30, 2022
(86) PCT No.: PCT/CN2022/095901

§ 371 Date: Jan. 11, 2023

(30) Foreign Application Priority Data
Jun. 1, 2021 (WO) PCT/CN2021/097679
Apr. 8, 2022 (WO) PCT/CN2022/085680

(51) Int. Cl. C07D 471/04; C07D 519/00

(52) U.S. Cl. CPC C07D 471/04; C07D 519/00; C07B 2200/07; C07B 2200/09; C07B 2200/05

(57) ABSTRACT

The present invention relates to pharmaceutical agents useful for therapy and/or prophylaxis in a mammal, pharmaceutical composition comprising such compounds, and their use as menin/MLL protein/protein interaction inhibitors, useful for treating diseases such as cancer, including but not limited to leukemia, myelodysplastic syndrome (MDS), and myeloproliferative neoplasms (MPN); and diabetes.

### FIELD OF THE INVENTION

The present invention relates to pharmaceutical agents useful for therapy and/or prophylaxis in a mammal, pharmaceutical composition comprising such compounds, and their use as menin/MLL protein/protein interaction inhibitors, useful for treating diseases such as cancer, including but not limited to leukemia, myelodysplastic syndrome (MDS), and myeloproliferative neoplasms (MPN); and diabetes.

### BACKGROUND OF THE INVENTION

Chromosomal rearrangements affecting the mixed lineage leukemia gene (MLL; MLL1; KMT2A) result in aggressive acute leukemias across all age groups and still represent mostly incurable diseases emphasizing the urgent need for novel therapeutic approaches. Acute leukemias harboring these chromosomal translocations of MLL represent as lymphoid, myeloid or biphenotypic disease and constitute 5 to 10% of acute leukemias in adults and approximately 70% in infants (Marschalek, Br J Haematol 2011. 152(2), 141-54; Tomizawa et al., Pediatr Blood Cancer 2007. 49(2), 127-32).

MLL is a histone methyltransferase that methylates histone H3 on lysine 4 (H3K4) and functions in multiprotein complexes. Use of inducible loss-of-function alleles of Mll1 demonstrated that Mll1 plays an essential role in sustaining hematopoietic stem cells (HSCs) and developing B cells although its histone methyltransferase activity is dispensable for hematopoiesis (Mishra et al., Cell Rep 2014. 7(4), 1239-47).

Fusion of MLL with more than 60 different partners has been reported to date and has been associated with leukemia formation/progression (Meyer et al., Leukemia 2013. 27, 2165-2176). Interestingly, the SET (Su(var)3-9, enhancer of zeste, and trithorax) domain of MLL is not retained in chimeric proteins but is replaced by the fusion partner (Thiel et al., Bioessays 2012. 34, 771-80). Recruitment of chromatin modifying enzymes like Dot1 L and/or the pTEFb complex by the fusion partner leads to enhanced transcription and transcriptional elongation of MLL target genes including HOXA genes (e.g. HOXA9) and the HOX cofactor MEIS1 as the most prominent ones. Aberrant expression of these genes in turn blocks hematopoietic differentiation and enhances proliferation.

Menin which is encoded by the Multiple Endocrine Neoplasia type 1 (MEN1) gene is expressed ubiquitously and is predominantly localized in the nucleus. It has been shown to interact with numerous proteins and is, therefore, involved in a variety of cellular processes. The best understood function of menin is its role as an oncogenic cofactor of MLL fusion proteins. Menin interacts with two motifs within the N-terminal fragment of MLL that is retained in all fusion proteins, MBM1 (menin-binding motif 1) and MBM2 (Thiel et al., Bioessays 2012. 34, 771-80). Menin/MLL interaction leads to the formation of a new interaction surface for lens epithelium-derived growth factor (LEDGF). Although MLL directly binds to LEDGF, menin is obligatory for the stable interaction between MILL and LEDGF and the gene specific chromatin recruitment of the MLL complex via the PWWP domain of LEDGF (Cermakova et al., Cancer Res 2014. 15, 5139-51; Yokoyama & Cleary, Cancer Cell 2008. 8, 36-46). Furthermore, numerous genetic studies have shown that menin is strictly required for oncogenic transformation by MLL fusion proteins suggesting the menin/MLL interaction as an attractive therapeutic target. For example, conditional deletion of Men1 prevents leukomogenesis in bone marrow progenitor cells ectopically expressing MLL fusions (Chen et al., Proc Natl Acad Sci 2006. 103, 1018-23). Similarly, genetic disruption of menin/MLL fusion interaction by loss-of-function mutations abrogates the oncogenic properties of the MLL fusion proteins, blocks the development of leukemia in vivo and releases the differentiation block of MLL-transformed leukemic blasts. These studies also showed that menin is required for the maintenance of HOX gene expression by MLL fusion proteins (Yokoyama et al., Cell 2005. 123, 207-18). In addition, small molecule inhibitors of menin/MLL interaction have been developed suggesting druggability of this protein/protein interaction and have also demonstrated efficacy in preclinical models of AML (Borkin et al., Cancer Cell 2015. 27, 589-602; Cierpicki and Grembecka, Future Med Chem 2014. 6, 447-462). Together with the observation that menin is not a requisite cofactor of MLL1 during normal hematopoiesis (Li et al., Blood 2013. 122, 2039-2046), these data validate the disruption of menin/MLL interaction as a promising new therapeutic approach for the treatment of MLL rearranged leukemia and other cancers with an active HOX/MEI gene signature. For example, an internal partial tandem duplication (PTD) within the 5′region of the MLL gene represents another major aberration that is found predominantly in de novo and secondary AML as well as myeloid dysplasia syndromes. Although the molecular mechanism and the biological function of MLL-PTD is not well understood, new therapeutic targeting strategies affecting the menin/MLL interaction might also prove effective in the treatment of MLL-PTD-related leukemias. Furthermore, castration-resistant prostate cancer has been shown to be dependent on the menin/MLL interaction (Malik et al., Nat Med 2015. 21, 344-52).

MLL protein is also known as Histone-lysine N-methyltransferase 2A (KMT2A) protein in the scientific field (UniProt Accession #Q03164).

Several references describe inhibitors targeting the menin-MLL interaction: WO2011029054, J Med Chem 2016, 59, 892-913 describe the preparation of thienopyrimidine and benzodiazepine derivatives; WO2014164543 describes thienopyrimidine and thienopyridine derivatives; Nature Chemical Biology March 2012, 8, 277-284 and Ren, J.; et al. Bioorg Med Chem Lett (2016), 26(18), 4472-4476 describe thienopyrimidine derivatives: J Med Chem 2014, 57, 1543-1556 describes hydroxy- and aminomethylpiperidine derivatives; Future Med Chen 2014, 6, 447-462 reviews small molecule and peptidomimetic compounds; WO2016195776 describes furo[2,3-d]pyrimidine, 9H-purine, [1,3]oxazolo[5,4-d]pyrimidine, [1,3]oxazolo[4,5-d]pyrimidine, [1,3]thiazolo[5,4-d]pyrimidine, thieno[2,3-b]pyridine and thieno[2,3-d]pyrimidine derivatives; WO2016197027 describes 5,6,7,8-tetrahydropyrido[3,4-d]pyrimidine, 5,6,7,8-tetrahydropyrido[4,3-d]pyrimidine, pyrido[2,3-d]pyrimidine and quinoline derivatives; and WO2016040330 describes thienopyrimidine and thienopyridine compounds. WO2017192543 describes piperidines as Menin inhibitors. WO2017112768, WO2017207387, WO2017214367, WO2018053267 and WO2018024602 describe inhibitors of the menin-MLL interaction. WO2017161002 and WO2017161028 describe inhibitors of menin-MLL. WO2018050686, WO2018050684 and WO2018109088 describe inhibitors of the menin-MLL interaction. WO2018226976 describes methods and compositions for inhibiting the interaction of menin with MLL proteins. WO2018175746 provides methods of treatment for hematological malignancies and Ewing's sarcoma. WO2018106818 and WO2018106820 provide methods of promoting proliferation of a pancreatic cell. WO2018153312 discloses azaspiro compounds relating to the field of medicinal chemistry. WO2017132398 discloses methods comprising contacting a leukemia cell exhibiting an NPM1 mutation with a pharmacologic inhibitor of interaction between MLL and Menin. WO2019060365 describes substituted inhibitors of menin-MLL. WO2020069027 describes the treatment of hematological malignancies with inhibitors of menin. Krivtsov et al., Cancer Cell 2019. No. 6 Vol. 36, 660-673 describes a menin-MLL inhibitor.

WO2014199171 discloses compounds as VAP1 inhibitors. WO2011113798 and WO2013037411 disclose compounds as SSAO inhibitors. WO2011056440 discloses compounds as CCR1 inhibitors.

WO2021060453 describes a crosslinking-type optically-active secondary amine derivative. WO2021121327 describes substituted straight chain spiro derivatives and their use as menin/MLL protein/protein interaction inhibitors.

### DESCRIPTION OF THE INVENTION

The present invention concerns novel compounds of Formula (I),

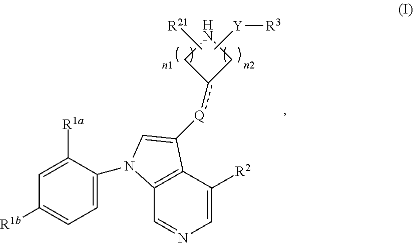

and the tautomers and the stereoisomeric forms thereof, wherein

Q represents —CHR^{y}—, —O—, —C(═O)—, —NR^{q}—, or —CR^{y}═; the dotted line is an optional additional bond to form a double bond in case Q represents —CR^{y}═;

R^{1a} represents hydrogen; cyano; halo; Het; —C(═O)—NR^{xa}R^{xb}; —S(═O)₂—R⁸; —C(═O)—O—C₁₋₄alkyl-NR^{22a}R^{22b}; —C(═O)—O—C₁₋₄alkyl;

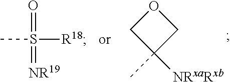

R¹⁸ represents C₁₋₆alkyl or C₃₋₆cycloalkyl;
R¹⁹ represents hydrogen or C₁₋₆alkyl;
or R¹⁸ and R¹⁹ are taken together to form —(CH₂)₃—, —(CH₂)₄— or —(CH₂)₅—;

Het represents a monocyclic 5- or 6-membered aromatic ring containing one, two or three O-, S- or N-atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;

R^{xa} and R^{xb} are each independently selected from the group consisting of hydrogen; Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, —C₁₋₄alkyl-OH, halo, CF₃, C₃₋₆cycloalkyl, Het³, and NR^{11c}—R^{11d};

or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents selected from the group consisting of halo and OR²³;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents each independently selected from the group consisting of halo and OR²³;
R²³ represents hydrogen or C₁₋₄alkyl optionally substituted with one, two or three halo;

R^{1b} represents hydrogen, F, Cl, or —O—C₁₋₄alkyl;
R² represents halo, C₃₋₆cycloalkyl, C₁₋₄alkyl, —O—C₁₋₄alkyl, cyano, or C₁₋₄alkyl substituted with one, two or three halo substituents;
R² represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;

Y and Y^{a} each independently represent a covalent bond or

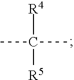

n1 is selected from 1 and 2;

n2 is selected from 1, 2, 3 and 4;

R^{y} represents hydrogen, —OH, C₁₋₄alkyl, —C₁₋₄alkyl-OH, or —C₁₋₄alkyl-O—C₁₋₄alkyl;

R^{q} represents hydrogen or C₁₋₄alkyl;

R⁵ represents hydrogen, C₁₋₄alkyl, or C₃₋₆cycloalkyl;

R³, R^{3a}, and R⁴ are each independently selected from the group consisting of Het¹; Het²; Cy²; C₁₋₈alkyl; and C₁₋₈alkyl substituted with one, two, three or four substituents each independently selected from the group consisting of —C(═O)—NR^{10a}R^{10b}, —C(═O)—Het^{6a}, —C(═O)—Het^{6b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², Ar¹, and Cy²;

R^{xc} represents Cy¹; Het⁵; —C₁₋₆alkyl-Cy¹; —C₁₋₆alkyl-Het³; —C₁₋₆alkyl-Het⁴; or —C₁₋₆alkyl-phenyl;

R^{xd} represents hydrogen; C₁₋₄alkyl; or C₁₋₄alkyl substituted with one, two or three substituents selected from the group consisting of halo, —OH, —O—C₁₋₄alkyl, and cyano;

or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;

R^{8a} and R^{8b} are each independently selected from the group consisting of hydrogen; C₁₋₆alkyl; —(C═O)—C₁₋₄alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl;

Ar¹ represents phenyl optionally substituted with one, two or three substituents each independently selected from the group consisting of C₁₋₄alkyl, halo, —O—C₁₋₄alkyl, —CF₃, —OH, —S(═O)₂—C₁₋₄alkyl, and —C(═O)—NR^{10a}R^{10b};

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂ wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;

Het² represents C-linked pyrazolyl, 1,2,4-oxadiazolyl, pyridazinyl or triazolyl; which may be optionally substituted on one nitrogen atom with R^{6a};

R⁶ and R^{6a} are each independently selected from the group consisting of

Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;

C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—N(C₁₋₄alkyl)₂, —C(═O)—NH—C₁₋₄alkyl-C₃₋₆cycloalkyl, —C(═O)—OH, —NR^{11a}R^{11b}, and —NH—S(═O)₂—C₁₋₄alkyl; and

C₃₋₆cycloalkyl optionally substituted by one or two substituents each independently selected from the group consisting of —CN, —OH, —O—C₁₋₄alkyl, —C(═O)—NH—C₁₋₄alkyl,

—C(═O)—N(C₁₋₄alkyl)₂, —NH—S(═O)₂—C₁₋₄alkyl, and C₇₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;

R⁸ represents hydrogen, —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, —S(═O)₂—C₁₋₄alkyl, Het^{3a}, and Het^{6a};

Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl or —(C═O)—C₁₋₄alkyl;

Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b}.

Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo, —NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};

Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b} and C₁₋₄alkyl;

Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;

Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, —O—C₁₋₄alkyl, cyano,

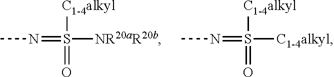

and

C₁₋₄alkyl substituted with one or two substituents each independently selected from the group consisting of Het^{3a}, Het^{6a}, Het^{6b}, and —NR^{9a}R^{9b};

Cy³ represents C₃₋₇cycloalkyl; wherein said C₃₋₇cycloalkyl is optionally substituted with one, two or three halo substituents;

R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵; Het⁷; —C₁₋₄alkyl-R¹⁶; —C(═O)—C₇₋₄alkyl-Het^{3a}; —C(═O)—R¹⁴;

C₃₋₆cycloalkyl substituted with one, two or three substituents selected from the group consisting of halo, —OH, —O—C₁₋₄alkyl, —NR^{11a}R^{11b}, and cyano; and

C₁₋₄alkyl substituted with one, two or three substituents selected from the group consisting of halo, —OH, —O—C₁₋₄alkyl, —NR^{11a}R^{11b}, and cyano;

R^{11a}, R^{11b}, R^{13a}, R^{13b}, R^{15a}, R^{15b}, R^{17a}, R^{17b}, R^{20a}, R^{20b}, R^{22a} and R^{22b} are each independently selected from the group consisting of hydrogen and C₁₋₄alkyl;

R^{11c} and R^{11d} are each independently selected from the group consisting of hydrogen, C₁₋₆alkyl, and —C(═O)—C₁₋₄alkyl;

R^{10a}, R^{10b} and R^{10c} are each independently selected from the group consisting of hydrogen, C₁₋₄alkyl, and C₃₋₆cycloalkyl;

R^{10d} and R^{10e} are each independently selected from the group consisting of C₁₋₄alkyl, —O—C₁₋₄alkyl and C₃₋₆cycloalkyl;

R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;

R¹⁶ represents —C(═O)—NR^{17a}R^{17b}, —S(═O)₂—C₁₋₄alkyl, Het⁵, Het⁷, or Het⁸;

and the pharmaceutically acceptable salts and the solvates thereof.

It should be clear that substituents R²¹ and —Y—R³ in Formula (I) can be attached to any carbon or nitrogen atom of the ring to which they are attached, thereby replacing hydrogens on the same atom or they may replace hydrogen atoms on different atoms in the moiety (including the N-atom). Lines drawn from substituents into ring systems indicate that the bond may be attached to any of the suitable ring atoms.

The present invention also concerns novel compounds of Formula (A),

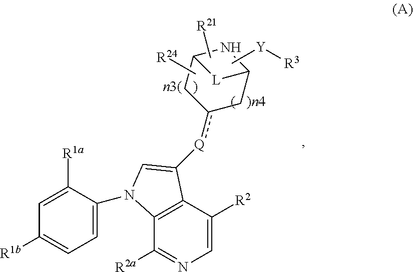

and the tautomers and the stereoisomeric forms thereof wherein

L is absent or represents —CH₂— or —CH₂—CH₂—;

Q represents —CHR^{y}—, —O—, —C(═O)—, —NR^{q}—, or —CR^{y}═; the dotted line is an optional additional bond to form a double bond in case Q represents —CR^{y}═;

R^{1a} represents hydrogen; cyano; halo; Het; —C(═O)—NR^{xa}R^{xb}; —S(═O)₂—R¹⁸; —C(═O)—O—C₁₋₄alkyl-NR^{22a}R^{22b}; —C(═O)—O—C₁₋₄alkyl;

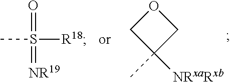

R¹⁸ represents C₁₋₆alkyl or C₃₋₆cycloalkyl;

R¹⁹ represents hydrogen or C₁₋₆alkyl;

or R¹⁸ and R¹⁹ are taken together to form —(CH₂)₃—, —(CH₂)₄— or —(CH₂)₅—;

Het represents a monocyclic 5- or 6-membered aromatic ring containing one, two or three O-, S- or N-atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, halo or cyano;

R^{xa} and R^{xb} are each independently selected from the group consisting of hydrogen; Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, —C₁₋₄alkyl-OH, halo, CF₃, C₃₋₆cycloalkyl, Het³, and NR^{11c}R^{11d}; or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents selected from the group consisting of halo and OR²³;

or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents each independently selected from the group consisting of halo and OR²³;

R²³ represents hydrogen or C₁₋₄alkyl optionally substituted with one, two or three halo;

R^{1b} represents hydrogen, F, Cl, or —O—C₁₋₄alkyl;

R² represents halo, C₃₋₆cycloalkyl, C₁₋₄alkyl, —O—C₁₋₄alkyl, cyano, or C₁₋₄alkyl substituted with one, two or three halo substituents;

R^{2a} represents hydrogen or C₁₋₄alkyl;

R²¹ represents hydrogen or —Y^{a}—R^{3a}; provided that when R^{2′} represents —Y^{a}—R^{3a}, one of —Y^{a}—R^{3a} and —Y—R³ is attached to the nitrogen atom of the ring;

Y and Y^{a} each independently represent a covalent bond or

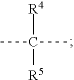

n3 is selected from 0 and 1;

n4 is selected from 0, 1, 2 and 3;

R^{y} represents hydrogen, —OH, C₁₋₄alkyl, —C₁₋₄alkyl-OH, or —C₁₋₄alkyl-O—C₁₋₄alkyl;

R^{q} represents hydrogen or C₁₋₄alkyl;

R⁵ represents hydrogen, C₁₋₄alkyl, or C₃₋₆cycloalkyl;

R³, R^{3a}, and R⁴ are each independently selected from the group consisting of Het¹; Het²; Cy²; C₁₋₈alkyl; and C₁₋₈alkyl substituted with one, two, three or four substituents each independently selected from the group consisting of —C(═O)—NR^{10a}R^{10b}, —C(═O)—Het^{6a}, —C(═O)—Het^{6b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², Ar¹, and Cy²;

R^{xc} represents Cy¹; Het⁵; —C₁₋₆alkyl-Cy¹; —C₁₋₆alkyl-Het³; —C₁₋₆alkyl-Het⁴;

or —C₁₋₆alkyl-phenyl;

R^{xd} represents hydrogen; C₁₋₄alkyl; or C₁₋₄alkyl substituted with one, two or three substituents selected from the group consisting of halo, —OH, —O—C₁₋₄alkyl, and cyano;

or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;

R^{8a} and R^{8b} are each independently selected from the group consisting of hydrogen; C₁₋₆alkyl; —(C═O)—C₁₋₄alkyl; and C₁₋₆alkyl substituted with one, two or three substituents each independently selected from the group consisting of —OH, cyano, halo, —S(═O)₂—C₁₋₄alkyl, —O—C₁₋₄alkyl,

—C(═O)—NR^{10a}R^{10b}, and —NR^{10c}—C(═O)—C₁₋₄alkyl;

Ar¹ represents phenyl optionally substituted with one, two or three substituents each independently selected from the group consisting of C₁₋₄alkyl, halo, —O—C₁₋₄alkyl, —CF₃, —OH, —S(═O)₂—C₁₋₄alkyl, and —C(═O)—NR^{10a}R^{10b}.

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂ wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;

Het² represents C-linked pyrazolyl, 1,2,4-oxadiazolyl, pyridazinyl or triazolyl; which may be optionally substituted on one nitrogen atom with R^{6a};

R⁶ and R^{6a} are each independently selected from the group consisting of

Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;

C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—N(C₁₋₄alkyl)₂, —C(═O)—NH—C₁₋₄alkyl-C₃₋₆cycloalkyl, —C(═O)—OH, —NR^{11a}R^{11b}, and

—NH—S(═O)₂—C₁₋₄alkyl; and

C₃₋₆cycloalkyl optionally substituted by one or two substituents each independently selected from the group consisting of —CN, —OH, —O—C₁₋₄alkyl, —C(═O)—NH—C₁₋₄alkyl, —C(═O)—N(C₁₋₄alkyl)₂, —NH—S(═O)₂—C₁₋₄alkyl, and C₁₋₄alkyl optionally substituted with one substituent selected from the group consisting of OH, —O—C₁₋₄alkyl, —C(═—O)—NH—C₁₋₄alkyl and —NH—S(═O)₂—C₁₋₄alkyl;

R⁸ represents hydrogen, —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, —S(═O)₂—C₁₋₄alkyl, Het^{3a}, and Het^{6a};

Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one carbon atom with C₁₋₄alkyl, halo, —OH, —NR^{11a}R^{11b} or oxo; and wherein said heterocyclyl is optionally substituted on one nitrogen atom with C₁₋₄alkyl or —(C═O)—C₁₋₄alkyl;

Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH₄—C(═O)—O—C₁₋₄alkyl, —NH—C(═O)—Cy³, —NH₁—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b}. Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo, —NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};

Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—N^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b}, and C₁₋₄alkyl;

Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;

Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, —O—C₁₋₄alkyl, cyano,

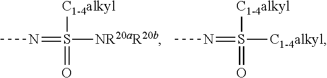

and

C₁₋₄alkyl substituted with one or two substituents each independently selected from the group consisting of Het^{3a}, Het^{6a}, Het^{6b}, and —NR^{9a}R^{9b};

Cy³ represents C₃₋₇cycloalkyl; wherein said C₃₋₇cycloalkyl is optionally substituted with one, two or three halo substituents;

R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵; Het⁷; —C₁₋₄alkyl-R¹⁶; —C(═O)—C₁₋₄alkyl-Het^{3a}; —C(═O)—R¹⁴; C₃₋₆cycloalkyl substituted with one, two or three substituents selected from the group consisting of halo, —OH, —O—C₁₋₄alkyl, —NR^{11a}R^{11b}, and cyano; and

C₁₋₄alkyl substituted with one, two or three substituents selected from the group consisting of halo, —OH, —O—C₁₋₄alkyl, —NR^{11a}R^{11b}, and cyano;

R^{11a}, R^{11b}, R^{13a}, R^{13b}, R^{5a}, R^{15b}, R^{17a}, R^{17b}, R^{20a}, R^{20b}, R^{22a}, and R^{22b} are each independently selected from the group consisting of hydrogen and C₁₋₄alkyl;

R^{11c} and R^{11d} are each independently selected from the group consisting of hydrogen, C₁₋₆alkyl, and —C(═O)—C₁₋₄alkyl;

R^{10a}, R^{10b} and R^{10c} are each independently selected from the group consisting of hydrogen, C₁₋₄alkyl, and C₃₋₆cycloalkyl;

R^{10d} and R^{10e} are each independently selected from the group consisting of C₁₋₄alkyl, —O—C₁₋₄alkyl and C₃₋₆cycloalkyl;

R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;

R¹⁶ represents —C(═O)—NR^{17a}R^{17b}, —S(═O)₂—C₁₋₄alkyl, Het⁵, Het⁷, or Het⁸;

R²⁴ represents hydrogen or C₁₋₄alkyl;

and the pharmaceutically acceptable salts and the solvates thereof.

It should be clear that substituents R²¹, R²⁴ and —Y—R³ in Formula (A) can be attached to any carbon or nitrogen atom of the ring to which they are attached, thereby replacing hydrogens on the same atom or they may replace hydrogen atoms on different atoms (including the N-atom) in the moiety. Lines drawn from substituents into ring systems indicate that the bond may be attached to any of the suitable ring atoms.

The present invention also relates to a pharmaceutical composition comprising a therapeutically effective amount of a compound of Formula (I), a pharmaceutically acceptable salt, or a solvate thereof.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer, including but not limited to leukemia, myelodysplastic syndrome (MDS), and myeloproliferative neoplasms (MPN); and diabetes.

In a particular embodiment, the invention relates to a compound of Formula (I), a pharmaceutically acceptable salt, or a solvate thereof, for use in the treatment or in the prevention of cancer.

In a specific embodiment said cancer is selected from leukemias, lymphomas, myelomas or solid tumor cancers (e.g. prostate cancer, lung cancer, breast cancer, pancreatic cancer, colon cancer, liver cancer, melanoma and glioblastoma, etc.). In some embodiments, the leukemias include acute leukemias, chronic leukemias, myeloid leukemias, myelogeneous leukemias, lymphoblastic leukemias, lymphocytic leukemias, Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, leukemias exhibiting HOX/MEIS1 gene expression signatures etc.

In particular, compounds according to the present invention and the pharmaceutical compositions thereof may be useful in the treatment or prevention of leukemias, in particular nucleophosmin (NPM1)-mutated leukemias, e.g. NPM1c.

In an embodiment, compounds of Formula (I) and the pharmaceutically acceptable salts, and the solvates thereof, may have improved metabolic stability properties.

In an embodiment, compounds of Formula (I) and the pharmaceutically acceptable salts, and the solvates thereof, may have extended in vivo half-life (T½).

In an embodiment, compounds of Formula (I) and the pharmaceutically acceptable salts, and the solvates thereof, may have improved oral bioavailability.

In an embodiment, compounds of Formula (I) and the pharmaceutically acceptable salts, and the solvates thereof, may reduce tumor growth e.g., tumours harbouring MLL (KMT2A) gene rearrangements/alterations and/or NPM1 mutations.

In an embodiment, compounds of Formula (I) and the pharmaceutically acceptable salts, and the solvates thereof, may have improved PD properties in vivo during a prolonged period of time, e.g. inhibition of target gene expression such as MEIS1 and upregulation of differentiation marker over a period of at least 16 hours.

In an embodiment, compounds of Formula (I) and the pharmaceutically acceptable salts, and the solvates thereof, may have an improved safety profile (e.g. reduced hERG inhibition; improved cardiovascular safety).

In an embodiment, compounds of Formula (I) and the pharmaceutically acceptable salts, and the solvates thereof, may be suitable for Q.D. dosing (once daily).

The invention also relates to the use of a compound of Formula (I), a pharmaceutically acceptable salt, or a solvate thereof, in combination with an additional pharmaceutical agent for use in the treatment or prevention of cancer, including but not limited to leukemia, myelodysplastic syndrome (MDS), and myeloproliferative neoplasms (MPN); and diabetes.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer, including but not limited to leukemia, myelodysplastic syndrome (MDS), and myeloproliferative neoplasms (MPN); and diabetes.

Additionally, the invention relates to a method of treating or preventing a cell proliferative disease in a warm-blooded animal which comprises administering to the said animal an effective amount of a compound of Formula (I), a pharmaceutically acceptable salt, or a solvate thereof, as defined herein, or a pharmaceutical composition or combination as defined herein.

Any aspects of the invention and embodiments described herein for the compounds of formula (I) as listed herein, also hold for the compounds of formula (A).

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific disclosure of the drawings. In the drawings:

FIG. 1 is an X-ray powder diffraction (XRPD) pattern of Compound 51 as a crystalline free base Form.

FIG. 2 is an X-ray powder diffraction (XRPD) pattern of Compound 51a as a crystalline HCl salt Form.

FIG. 3 is a Dynamic vapor sorption (DVS) isotherm plot of Compound 51a as a crystalline HCl salt Form.

FIG. 4 is a Dynamic vapor sorption (DVS) change in mass plot of Compound 51a as a crystalline HCl salt Form.

### DETAILED DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain saturated hydrocarbon radical having from 1 to 4 carbon atoms, such as methyl, ethyl, n-propyl, isopropyl, n-butyl, s-butyl, 1-butyl and the like.

Similar, the term ‘C₁₋₆alkyl’ as used herein as a group or part of a group represents a straight or branched chain saturated hydrocarbon radical having from 1 to 6 carbon atoms, such as methyl, ethyl, n-propyl, isopropyl, n-butyl, s-butyl, t-butyl, n-pentyl, n-hexyl and the like.

Similar, the term ‘C₁₋₈alkyl’ as used herein as a group or part of a group represents a straight or branched chain saturated hydrocarbon radical having from 1 to 8 carbon atoms, such as methyl, ethyl, n-propyl, isopropyl, n-butyl, s-butyl, t-butyl, n-pentyl, n-hexyl,

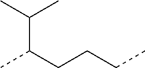

and the like.

The term ‘C₃₋₆cycloalkyl’ as used herein as a group or part of a group defines a saturated, cyclic hydrocarbon radical having from 3 to 6 carbon atoms, such as cyclopropyl, cyclobutyl, cyclopentyl and cyclohexyl.

The term ‘C₃₋₇cycloalkyl’ as used herein as a group or part of a group defines a saturated, cyclic hydrocarbon radical having from 3 to 7 carbon atoms, such as cyclopropyl, cyclobutyl, cyclopentyl, cyclohexyl and cycloheptyl.

It will be clear for the skilled person that S(═O)₂ or SO₂ represents a sulfonyl moiety.

It will be clear for the skilled person that CO or C(═O) represents a carbonyl moiety.

It will be clear for the skilled person that a group such as —NR— represents

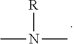

An example of such a group is —NR^{q}—.

Non-limiting examples of ‘monocyclic 5- or 6-membered aromatic rings containing one, two or three nitrogen atoms and optionally a carbonyl moiety’, include, but are not limited to pyrazolyl, imidazolyl, pyridinyl, pyridazinyl, pyrimidinyl, pyrazinyl, triazinyl or 1,2-dihydro-2-oxo-4-pyridinyl.

The skilled person will understand that a monocyclic 5- or 6-membered aromatic ring containing one, two or three nitrogen atoms and a carbonyl moiety includes, but is not limited to

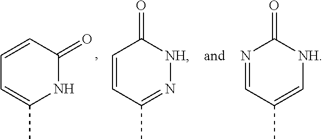

The term ‘monocyclic N-linked 4- to 7-membered fully or partially saturated heterocyclyl containing one N-atom and optionally one or two additional heteroatoms each independently selected from O, S, and N’, defines a fully or partially saturated, cyclic hydrocarbon radical having from 4 to 7 ring members and containing at least 1 nitrogen atom and optionally one or two additional heteroatoms each independently selected from O, S, and N, which is attached to the remainder of the molecule of formula (I) via a nitrogen atom. Examples are N-linked azetidinyl, N-linked pyrrolidinyl, N-linked morpholinyl, N-linked thiomorpholinyl, N-linked piperazinyl, N-linked 1,4-diazepanyl, N-linked piperidinyl, and N-linked 1,2,3,6-tetrahydro-pyridinyl. Two R groups taken together to form together with the N-atom to which they are attached a 4- to 7-membered monocyclic fully or partially saturated heterocyclyl containing one N-atom and optionally one additional heteroatom selected from O, S, and N, are defined similar.

The term ‘monocyclic C-linked 4- to 7-membered fully or partially saturated heterocyclyl containing one, two or three heteroatoms each independently selected from O, S, and N’, defines a fully or partially saturated, cyclic hydrocarbon radical having from 4 to 7 ring members and containing one, two or three heteroatoms each independently selected from O, S, and N, such as for example C-linked azetidinyl, C-linked pyrrolidinyl, C-linked morpholinyl, C-linked tetrahydrofuranyl, C-linked thiolanyl, C-linked oxetanyl, C-linked thietanyl, C-linked tetrahydropyranyl, C-linked tetrahydrothiopyranyl, C-linked piperidinyl, C-linked azepanyl, C-linked 1,3-dioxolanyl, and C-linked 1,2,3,6-tetrahydro-pyridinyl.

For clarity, the 4- to 7-membered fully or partially saturated heterocyclyls have from 4 to 7 ring members including the heteroatoms.

Non-limiting examples of ‘monocyclic C-linked 5- or 6-membered aromatic rings containing one, two or three heteroatoms each independently selected from O, S, and N’, include, but are not limited to C-linked pyrazolyl, C-linked imidazolyl, C-linked pyridinyl, C-linked triazolyl, C-linked pyridazinyl, C-linked pyrimidinyl, C-linked oxazolyl, C-linked furanyl, C-linked isothiazolyl, C-linked thiazolyl, C-linked thiadiazolyl, C-linked oxadiazolyl, or C-linked pyrazinyl.

Within the context of this invention, bicyclic 6- to 11-membered fully or partially saturated heterocyclyl groups, include fused, spiro and bridged bicycles.

Fused bicyclic groups are two cycles that share two atoms and the bond between these atoms. Spiro bicyclic groups are two cycles that are joined at a single atom.

Bridged bicyclic groups are two cycles that share more than two atoms.

Examples of bicyclic C-linked 6- to 11-membered fully or partially saturated heterocyclyl containing one, two or three heteroatoms each independently selected from O, S, and N, include, but are not limited to

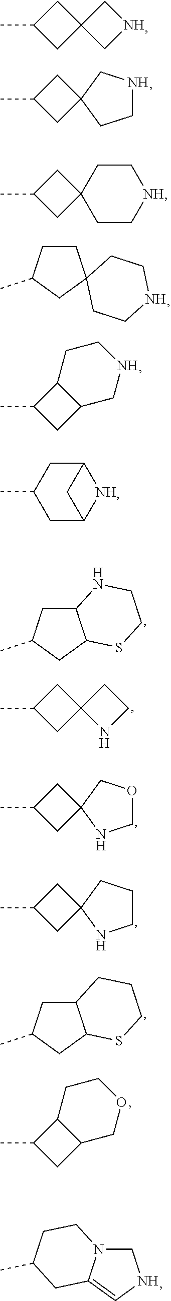

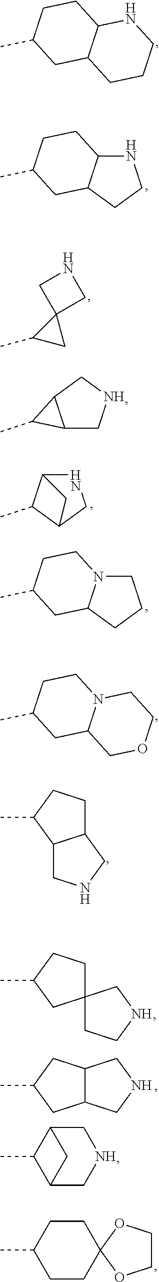

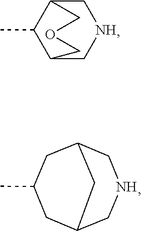

and the like.

Examples of bicy clic N-linked 6- to 11-membered fully or partially saturated heterocyclyl containing one N-atom and optionally one or two additional heteroatoms each independently selected from O, S, and N, include, but are not limited to

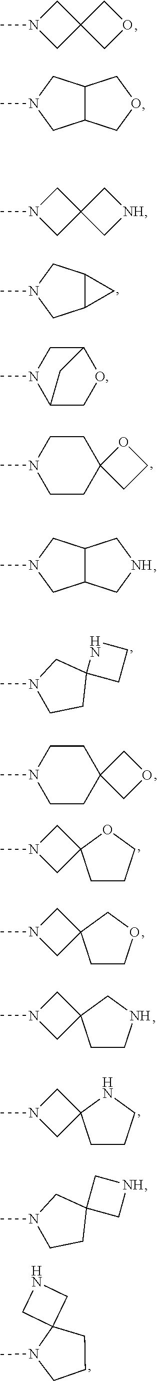

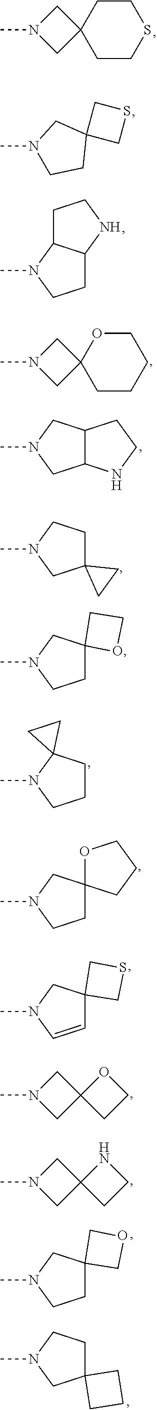

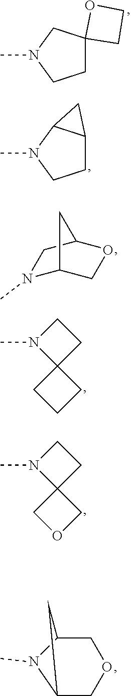

and the like.

Two R groups taken together to form together with the N-atom to which they are attached a 6- to 11-membered bicyclic fully or partially saturated heterocyclyl containing one N-atom and optionally one additional heteroatom selected from O, S, and N, are defined similar.

Examples of fused bicyclic C-linked 9- or 10-membered aromatic ring containing one, two, three or four heteroatoms each independently selected from O, S, and N, include but are not limited to

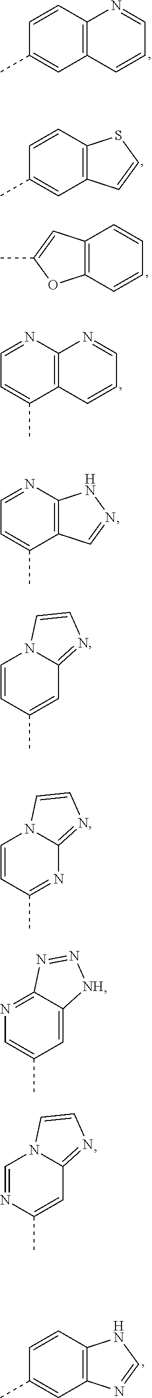

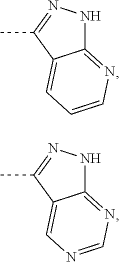

and the like.

As used herein ‘5- to 12-membered saturated carbobicyclic’ systems define saturated fused, spiro and bridged bicyclic hydrocarbon systems having from 5 to 12 carbon atoms. Examples of 5- to 12-membered saturated carbobicyclic’ systems include, but are not limited to

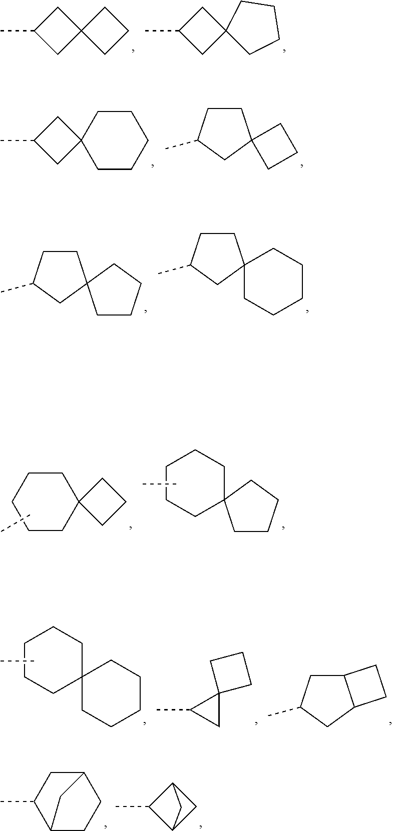

and the like.

Whenever substituents are represented by chemical structure, such as for example

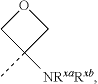

‘----’ represents the bond of attachment to the remainder of the molecule of Formula (I).

When any variable occurs more than one time in any constituent, each definition is independent.

When any variable occurs more than one time in any formula (e.g. Formula (I)), each definition is independent.

It will be clear for a skilled person that when a moiety (for example a heterocyclyl or monocyclic 5- or 6-membered aromatic ring) is substituted with two or more substituents (for example one, two or three substituents) selected from a group, each substituent can be selected independently from said group, even if not explicitly mentioned.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture (isolation after a reaction e.g. purification by silica gel chromatography). In a particular embodiment, when the number of substituents is not explicitly specified, the number of substituents is one.

Combinations of substituents and/or variables are permissible only if such combinations result in chemically stable compounds. ‘Stable compound’ is in this context meant to indicate a compound that is sufficiently robust to survive isolation to a useful degree of purity from a reaction mixture (isolation after a reaction e.g. purification by silica gel chromatography).

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

Within the context of this invention ‘saturated’ means ‘fully saturated’, if not otherwise specified.

Unless otherwise specified or clear from the context, aromatic rings and heterocyclyl groups, can be attached to the remainder of the molecule of Formula (I) through any available ring carbon atom (C-linked) or nitrogen atom (N-linked).

Unless otherwise specified or clear from the context, aromatic rings and heterocyclyl groups, may optionally be substituted, where possible, on carbon and/or nitrogen atoms according to the embodiments. A skilled person will understand that in such a case hydrogens on the carbon and/or nitrogen atoms are replaced by such substituents.

Unless otherwise specified or clear from the context, variable R²¹ and —Y—R³ can be attached to any carbon or nitrogen atom of the ring to which they are attached, provided that when R²¹ represents —Y^{a}—R^{3a}, one of —Y^{a}—R^{3a} and —Y—R³ is attached to the nitrogen atom of the ring.

For example in case R²¹ represents hydrogen, and —Y—R³ is attached to the nitrogen atom of the ring in Formula (I), a compound of subformula (I-x) is obtained:

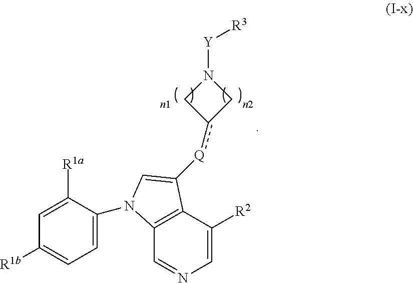

In case Y represents a covalent bond in Formula (I), a compound of subformula (I-y) is obtained:

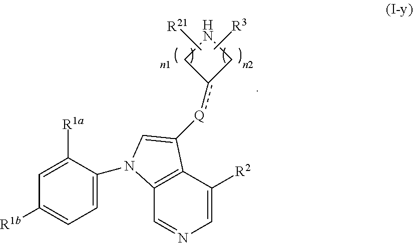

In case Y represents

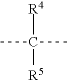

in Formula (I), a compound of subformula (I-z) is obtained:

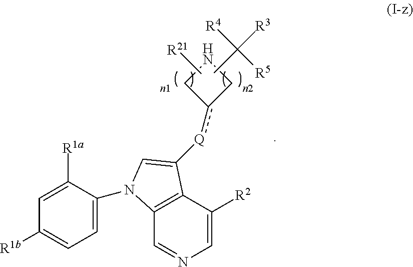

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not knon can be designated by (+) or (−) depending on the direction in which they rotate plane polarized light. For instance, resolved enantiomers whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer.

Some of the compounds according to Formula (I) may also exist in their tautomeric form. Such forms in so far as they may exist, although not explicitly indicated in the above Formula (I) are intended to be included within the scope of the present invention. It follows that a single compound may exist in both stereoisomeric and tautomeric form.

Pharmaceutically acceptable salts include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I) and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylanine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term “prodrug” includes any compound that, following oral or parenteral administration, in particular oral administration, is metabolised in vivo to a (more) active form in an experimentally-detectable amount, and within a predetermined time (e.g. within a dosing interval of between 0.5 and 24 hours, or e.g. within a dosing interval of between 6 and 24 hours (i.e. once to four times daily)). For the avoidance of doubt, the term “parenteral” administration includes all forms of administration other than oral administration, in particular intravenous (IV), intramuscular (IM), and subcutaneous (SC) injection.

Prodrugs may be prepared by modifying functional groups present on a compound in such a way that the modifications are cleaved in vivo when such prodrug is administered to a mammalian subject. The modifications typically are achieved by synthesising the parent compound with a prodrug substituent. In general, prodrugs include compounds wherein a hydroxyl, amino, sulfhydryl, carboxy or carbonyl group is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶C, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C, ¹³C and ¹⁸F. Preferably, the isotope is selected from the group of ²H, ³H, ¹¹C and ¹⁸F. More preferably, the isotope is ²H, ³H or ¹³C. More preferably, the isotope is ²H or ¹³C. More preferably, the isotope is ²H. In particular, deuterated compounds and ¹³C-enriched compounds are intended to be included within the scope of the present invention. In particular, deuterated compounds are intended to be included within the scope of the present invention.

Certain isotopically-labeled compounds of the present invention (e.g., those labeled with 3H and NC) may be useful for example in substrate tissue distribution assays. Tritiated (H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

The present invention relates in particular to compounds of Formula (I) as defined herein, and the tautomers and the stereoisomeric forms thereof, wherein

Q represents —CHR^{y}— or —CR^{y}═; the dotted line is an optional additional bond to form a double bond in case Q represents —CR^{y}═;

R^{1a} represents hydrogen; halo; —C(═O)—NR^{xa}R^{xb}; —S(═O)₂—R¹⁸;

—C(═O)—O—C₁₋₄alkyl; or

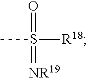

R¹⁸ represents C₁₋₆alkyl;

R¹⁹ represents hydrogen or C₁₋₆alkyl;

or R¹⁸ and R¹⁹ are taken together to form —(CH₂)₃—, —(CH₂)₄— or —(CH₂)₅—;

R^{xa} and R^{xb} are each independently selected from the group consisting of hydrogen;

Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and —C₁₋₄alkyl-OH;

or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, —O—C₁₋₄alkyl, and C₁₋₄alkyl substituted with one, two or three OR²³;

or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —OH substituents;

R²³ represents hydrogen or C₁₋₄alkyl;

R^{1b} represents F or —O—C₁₋₄alkyl;

R² represents halo, C₁₋₄alkyl, or C₁₋₄alkyl substituted with one, two or three halo substituents;

R²¹ represents hydrogen or —Y^{a}—R^{3a}; provided that when R¹ represents —Y^{a}—R^{3a}, one of —Y^{a}—R^{3a} and —Y—R³ is attached to the nitrogen atom of the ring;

Y and Y^{a} each independently represent a covalent bond or

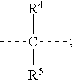

R⁵ represents hydrogen;

n1 is selected from 1 and 2;

n2 is selected from 1, 2 and 3;

R^{y} represents hydrogen;

R³, R^{3a}, and R⁴ are each independently selected from the group consisting of Het¹; C₁₋₈alkyl; and C₁₋₈alkyl substituted with one, two, three or four substituents each independently selected from the group consisting of —C(═O)-Het^{6a}, —C(═O)-Het^{6b}, —NR^{10c}—C(═O)—C₁₋₄alkyl, —NR^{xc}R^{xd}, —NR^{8a}R^{8b}, —CF₃, halo, —OH, —O—C₁₋₄alkyl, Het¹, Het², Ar¹, and Cy²;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl, and —S(═O)₂—C₁₋₄alkyl;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;

R^{8a} and R^{8b} are each independently selected from the group consisting of hydrogen; C₁₋₆alkyl; —(C═O)—C₁₋₄alkyl; and C₁₋₆alkyl substituted with one, two or three —O—C₁₋₄alkyl;

Ar¹ represents phenyl optionally substituted with one, two or three substituents each independently selected from the group consisting of C₁₋₄alkyl and —C(═O)—NR^{10a}R^{10b};

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, C₁₋₄alkyl, oxo and —OH;

Het² represents C-linked pyrazolyl, 1,2,4-oxadiazolyl, pyridazinyl or triazolyl;

R⁶ is selected from the group consisting of Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;

C₁₋₆alkyl optionally substituted with one or two substituents each independently selected from the group consisting of Het^{6a}, Het^{6b}, and —OH;

R⁸ represents hydrogen, —O—C₁₋₆alkyl, C₁₋₆alkyl; or C₁₋₆alkyl substituted with one, two or three substituents each independently selected from —OH, —O—C₁₋₄alkyl, cyano, —S(═O)₂—C₁₋₄alkyl, and Het^{3a};

Het³ and Het^{3a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen atom with —(C═O)—C₁₋₄alkyl;

Het⁴ represents a monocyclic C-linked 5- or 6-membered aromatic ring containing one, two or three heteroatoms each independently selected from O, S, and N; wherein said aromatic ring is optionally substituted on one or two carbon atoms with in total one or two substituents each independently selected from the group consisting of C₁₋₄alkyl and —C(═O)—NR^{10a}R^{10b}:

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo and —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;

Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with a —C(═O)—C₁₋₄alkyl;

Cy¹ represents C₃₋₆cycloalkyl optionally substituted with one, two or three —OH;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, and C₁₋₄alkyl;

R^{9a} and R^{9b} are each independently selected from the group consisting of hydrogen; C₁₋₄alkyl; —C(═O)—C₁₋₄alkyl; —S(═O)₂—C₁₋₄alkyl; and —C(═O)—R¹⁴;

R^{10a}, R^{10b} and R^{10c} are each independently selected from the group consisting of hydrogen and C₁₋₄alkyl;

R^{10d} and R^{10c} are each independently selected from the group consisting of C₁₋₄alkyl and —O—C₁₋₄alkyl;

R¹⁴ represents —O—C₁₋₄alkyl;

and the pharmaceutically acceptable salts and the solvates thereof.

The present invention relates in particular to compounds of Formula (I) as defined herein, and the tautomers and the stereoisomeric forms thereof, wherein

Q represents —CHR^{y}—;

R^{1a} represents —C(O)—NR^{xa}R^{xb}; —S(═O)₂—R¹⁸;

—C(═O)—O—C₁₋₄alkyl; or

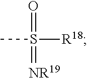

R¹⁸ represents C₁₋₆alkyl;

R¹⁹ represents hydrogen or C₁₋₆alkyl;

or R¹⁸ and R¹⁹ are taken together to form —(CH₂)₃—, —(CH₂)₄— or —(CH₂)₅—;

R^{xa} and R^{xb} are each independently selected from the group consisting of hydrogen;

Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and —C₁₋₄alkyl-OH;
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, —O—C₁₋₄alkyl, and C₁₋₄alkyl substituted with one, two or three OR²³;

or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —OH substituents;

R²³ represents hydrogen or C₁₋₄alkyl;

R^{1b} represents F or —O—C₁₋₄alkyl;

R² represents halo, C₁₋₄alkyl, or C₁₋₄alkyl substituted with one, two or three halo substituents;

R²¹ represents hydrogen or —Y^{a}—R^{3a}; provided that when R²¹ represents —Y^{a}—R₃ₐ, one of —Y^{a}—R^{3a} and —Y—R³ is attached to the nitrogen atom of the ring;

Y and Y^{a} represent a covalent bond;

n1 is selected from 1 and 2;

n2 is selected from 1, 2 and 3;

R^{y} represents hydrogen;

R³ and R^{3a} are each independently selected from the group consisting of Het¹; C₁₋₈alkyl; and C₁₋₈alkyl substituted with one, two, three or four substituents each independently selected from the group consisting of —C(═O)—Het^{6a}, —C(═O)—Het^{6b}, —NR^{10c}—C(═O)—C₁₋₄alkyl, —NR^{xc}R^{xd}, —NR^{8a}R^{8b}, —CF₃, halo, —OH, —O—C₁₋₄alkyl, Het¹, Het², Ar¹, and Cy²;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl, and —S(═O)₂—C₁₋₄alkyl;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;

R^{8a} and R^{8b} are each independently selected from the group consisting of hydrogen; C₁₋₆alkyl; —(C═O)—C₁₋₄alkyl; and C₁₋₆alkyl substituted with one, two or three —O—C₁₋₄alkyl;

Ar¹ represents phenyl optionally substituted with one, two or three substituents each independently selected from the group consisting of C₁₋₄alkyl and —C(═O)—NR^{10a}R^{10b};

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, C₁₋₄alkyl, oxo and —OH;

Het² represents C-linked pyrazolyl, 1,2,4-oxadiazolyl, pyridazinyl or triazolyl; R⁶ is selected from the group consisting of Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;

C₁₋₆alkyl optionally substituted with one or two substituents each independently selected from the group consisting of Het^{6a}, Het^{6b}, and —OH;

R⁸ represents hydrogen, —O—C₁₋₆alkyl, C₁₋₆alkyl; or C₁₋₆alkyl substituted with one, two or three substituents each independently selected from —OH, —O—C₁₋₄alkyl, cyano, —S(═O)₂—C₁₋₄alkyl, and Het^{3a};

Het³ and Het^{3a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen atom with —(C═O)—C₁₋₄alkyl;

Het⁴ represents a monocyclic C-linked 5- or 6-membered aromatic ring containing one, two or three heteroatoms each independently selected from O, S, and N; wherein said aromatic ring is optionally substituted on one or two carbon atoms with in total one or two substituents each independently selected from the group consisting of C₁₋₄alkyl and —C(═O)—NR^{10a}R^{10b};

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo and —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;

Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with a —C(═O)—C₁₋₄alkyl;

Cy¹ represents C₃₋₆cycloalkyl optionally substituted with one, two or three —OH;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het⁶, Het^{6b}, —NR^{9a}R^{9b}, —OH, and C₁₋₄alkyl;

R^{9a} and R^{9b} are each independently selected from the group consisting of hydrogen; C₁₋₄alkyl; —C(═O)—C₁₋₄alkyl; —S(═O)₂—C₁₋₄alkyl; and —C(═O)—R¹⁴;

R^{10a}, R^{10b} and R^{10c} are each independently selected from the group consisting of hydrogen and C₁₋₄alkyl;

R^{10d} and R^{10e} are each independently selected from the group consisting of C₁₋₄alkyl and —O—C₁₋₄alkyl;

R¹⁴ represents —O—C₁₋₄alkyl;

and the pharmaceutically acceptable salts and the solvates thereof.

The present invention relates in particular to compounds of Formula (I) as defined herein, and the tautomers and the stereoisomeric forms thereof, wherein

Q represents —CHR^{y}—;

R^{1a} represents —C(═O)—NR^{xa}R^{xb}; —S(═O)₂—R¹⁸;

—C(═O)—O—C₁₋₄alkyl; or

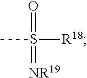

R¹⁸ represents C₁₋₆alkyl;

R¹⁹ represents hydrogen or C₁₋₆alkyl;

or R¹⁸ and R¹⁹ are taken together to form —(CH₂)₃—;

R^{xa} and R^{xb} are each independently selected from the group consisting of hydrogen; Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and —C₁₋₄alkyl-OH;

or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, —O—C₁₋₄alkyl, and C₁₋₄alkyl substituted with one, two or three OR²;

or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —OH substituents;

R²³ represents hydrogen or C₁₋₄alkyl;

R^{1b} represents F or —O—C₁₋₄alkyl;

R² represents halo, C₁₋₄alkyl, or C₁₋₄alkyl substituted with one, two or three halo substituents;

R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;

Y and Y^{a} each independently represent a covalent bond;

n1 is selected from 1 and 2:

n2 is selected from 1, 2 and 3:

R^{y} represents hydrogen;

R³ and R^{3a} are each independently selected from the group consisting of Het¹; C₁₋₈alkyl; and C₁₋₈alkyl substituted with one, two, three or four substituents each independently selected from the group consisting of —C(═O)-Het^{6a}, —C(═O)—Het^{6b}, —NR^{10c}—C(═O)—C₁₋₄alkyl, —NR^{xc}R^{xd}, —NR^{8a}R^{8b}, —CF₃, halo, —OH, —O—C₁₋₄alkyl, Het¹, Het², Ar¹, and Cy²;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl, and —S(═O)₂—C₁₋₄alkyl;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —(C═O)—C₁₋₄alkyl;

R^{8a} and R^{8b} are each independently selected from the group consisting of hydrogen; C₁₋₆alkyl; —(C═O)—C₁₋₄alkyl; and C₁₋₆alkyl substituted with one, two or three —O—C₁₋₄alkyl;

Ar¹ represents phenyl optionally substituted with one, two or three —C(═O)—NR^{10a}R^{10b}.

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, C₁₋₄alkyl, oxo and —OH;

Het² represents C-linked pyrazolyl, 1,2,4-oxadiazolyl, or pyridazinyl;

R⁶ is selected from the group consisting of Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;

C₁₋₆alkyl optionally substituted with one or two substituents each independently selected from the group consisting of Het^{6a}, Het^{6b}, and —OH;

R⁸ represents hydrogen, —O—C₁₋₆alkyl, C₁₋₆alkyl; or C₁₋₆alkyl substituted with one, two or three substituents each independently selected from —OH, —O—C₁₋₄alkyl, cyano and Het^{3a};

Het³ and Het^{3a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen atom with —(C═O)—C₁₋₄alkyl;

Het⁴ represents a monocyclic C-linked 5- or 6-membered aromatic ring containing one, two or three heteroatoms each independently selected from O, S, and N; wherein said aromatic ring is optionally substituted on one or two carbon atoms with in total one or two —C(═O)—NR^{10a}R^{10b}.

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four halo; and wherein said heterocyclyl is optionally substituted on one nitrogen with a substituent selected from the group consisting of —C(═O)—C₁₋₄alkyl and —S(═O)₂—C₁₋₄alkyl;

Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with a —C(═O)—C₁₋₄alkyl;

Cy¹ represents C₃₋₆cycloalkyl optionally substituted with one, two or three —OH;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, and C₁₋₄alkyl; R^{9a} and R^{9b} are each independently selected from the group consisting of hydrogen; C₁₋₄alkyl; —C(═)—C₁₋₄alkyl; —S(═O)₂—C₁₋₄alkyl; and —C(═O)—R¹⁴;

R^{10a}, R^{10b} and R^{10c} are each independently selected from the group consisting of hydrogen and C₁₋₄alkyl;

R^{10d} and R^{10e} are each independently selected from the group consisting of C₁₋₄alkyl and —O—C₁₋₄alkyl;

R¹⁴ represents —O—C₁₋₄alkyl;

and the pharmaceutically acceptable salts and the solvates thereof.

The present invention relates in particular to compounds of Formula (I) as defined herein, and the tautomers and the stereoisomeric forms thereof, wherein

Q represents —CHR^{y}— or —CR^{y}═; the dotted line is an optional additional bond to form a double bond in case Q represents —CR^{y}═;

R^{1a} represents hydrogen; halo; —C(═O)—NR^{xa}R^{xb}; —S(═O)₂—R¹⁸.

—C(═O)—O—C₁₋₄alkyl;

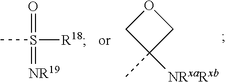

R¹⁸ represents C₁₋₆alkyl;

R¹⁹ represents hydrogen or C₁₋₆alkyl;

or R¹⁸ and R¹⁹ are taken together to form —(CH₂)₃—;

R^{xa} and R^{xb} are each independently selected from the group consisting of hydrogen; Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and —C₁₋₄alkyl-OH;

or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, and C₁₋₄alkyl substituted with one, two or three substituents selected from the group consisting of OR²³;

or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —OH substituents;

R²³ represents hydrogen or C₁₋₄alkyl;

R^{1b} represents F;

R² represents halo, C₁₋₄alkyl, or C₁₋₄alkyl substituted with one, two or three halo substituents;

R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;

Y and Y^{a} each independently represent a covalent bond or

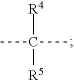

n1 is selected from 1 and 2;

n2 is selected from 1, 2 and 3;

R^{y} represents hydrogen;

R⁵ represents hydrogen;

R³, R^{3a}, and R⁴ are each independently selected from the group consisting of Het¹; Het²; Cy²; C₁₋₈alkyl; and C₁₋₈alkyl substituted with one, two, three or four substituents each independently selected from the group consisting of —C(═O)—Het^{6a}, —C(═O)—Het^{6b}, —NR^{10c}—C(═O)—C₁₋₄alkyl,

—NR^{xc}R^{xd}, —NR^{8a}R^{8b}, —CF₃, halo, —OH, —O—C₁₋₄alkyl, Het¹, Het², Ar¹, and Cy²;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;

R^{8a} and R^{8b} are each independently selected from the group consisting of hydrogen; C₁₋₆alkyl; and C₁₋₆alkyl substituted with one —O—C₁₋₄alkyl;

Ar¹ represents phenyl optionally substituted with one, two or three substituents each independently selected from the group consisting of C₁₋₄alkyl and —C(═O)—NR^{10a}R^{10b};

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, C₁₋₄alkyl, oxo, and —OH;

Het² represents C-linked pyrazolyl, 1,2,4-oxadiazolyl, pyridazinyl or triazolyl;

R⁶ is selected from the group consisting of Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;

C₁₋₆alkyl optionally substituted with one or two —OH substituents; and

C₃₋₆cycloalkyl;

R⁸ represents —O—C₁₋₆alkyl, C₁₋₆alkyl; or C₁₋₆alkyl substituted with one, two or three substituents each independently selected from —OH, —O—C₁₋₄alkyl, cyano, —S(═O)₂—C₁₋₄alkyl, and Het^{3a};

Het³ and Het^{3a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one carbon atom with oxo; and wherein said heterocyclyl is optionally substituted on one nitrogen atom with

—(C═O)—C₁₋₄alkyl;

Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two substituents each independently selected from the group consisting of C₁₋₄alkyl and —C(═O)—NR^{10a}R^{10b};

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo and —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;

Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with a —C(═O)—C₁₋₄alkyl;

Cy¹ represents C₃₋₆cycloalkyl optionally substituted with one, two or three —OH;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of R⁶, —C(═O)—Het^{6a}, Het^{6a}, Het^{6b}, —NR^{9a}R^{9b}, —OH, and C₁₋₄alkyl;

R^{9a} and R^{9b} are each independently selected from the group consisting of hydrogen; C₁₋₄alkyl; —C(═O)—C₁₋₄alkyl; —S(═O)₂—C₁₋₄alkyl; and —C(═O)—R¹⁴;

R^{10a}, R^{10b} and R^{10c} are each independently selected from the group consisting of hydrogen and C₁₋₄alkyl;

R^{10d} and R^{10e} are each independently selected from the group consisting of C₁₋₄alkyl and —O—C₁₋₄alkyl;

R¹⁴ represents —O—C₁₋₄alkyl;

and the pharmaceutically acceptable salts and the solvates thereof.

The present invention relates in particular to compounds of Formula (I) as defined herein, and the tautomers and the stereoisomeric forms thereof, wherein

Q represents —CHR^{y}—, —O—, —C(═O)—, —NR^{q}—, or —CR^{y}═; the dotted line is an optional additional bond to form a double bond in case Q represents —CR^{y}═;

R^{1a} represents hydrogen; cyano; halo; Het; —C(═O)—NR^{xa}R^{xb}; —S(═O)₂—R¹⁸;

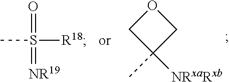

R¹⁸ represents C₁₋₆alkyl or C₃₋₆cycloalkyl;

R¹⁹ represents hydrogen or C₁₋₆alkyl;

Het represents a monocyclic 5- or 6-membered aromatic ring containing one, two or three nitrogen atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;

R^{xa} and R^{xb} are each independently selected from the group consisting of hydrogen; Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and NR^{11c}R^{11d};

or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;

or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;

R^{1b} represents hydrogen, F or Cl;

R² represents halo, C₃₋₆cycloalkyl, C₁₋₄alkyl, —O—C₁₋₄alkyl, cyano, or C₁₋₄alkyl substituted with one, two or three halo substituents;

R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;

Y and Y^{a} each independently represent a covalent bond or

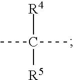

n1 and n2 are each independently selected from 1 and 2;

R^{y} represents hydrogen, —OH, C₁₋₄alkyl, —C₁₋₄alkyl-OH, or —C₁₋₄alkyl-O—C₁₋₄alkyl;

R^{q} represents hydrogen or C₁₋₄alkyl;

R⁵ represents hydrogen, C₁₋₄alkyl, or C₃₋₆cycloalkyl;

R³, R^{3a}, and R⁴ are each independently selected from the group consisting of Het¹; Het²; Cy²; C₁₋₆alkyl; and C₁₋₆alkyl substituted with one, two, three or four substituents each independently selected from the group consisting of —C(═O)—NR^{10a}R^{10b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;

R^{xc} represents Cy¹; Het⁵; —C₁₋₆alkyl-Cy¹; —C₁₋₆alkyl-Het³; —C₁₋₆alkyl-Het⁴; or —C₁₋₆alkyl-phenyl;

R^{xd} represents hydrogen; C₁₋₄alkyl; or C₁₋₄alkyl substituted with one, two or three substituents selected from the group consisting of halo, —OH, —O—C₁₋₄alkyl, and cyano;

or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;

R^{8a} and R^{8b} are each independently selected from the group consisting of hydrogen; C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl;

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;

Het² represents C-linked pyrazolyl or triazolyl; which may be optionally substituted on one nitrogen atom with R^{6a};

R⁶ and R^{6a} are each independently selected from the group consisting of

Het³; Het⁴; —C(═O)—NH—Cy¹; —C(═O)—NH—R^{B}; —S(═O)₂—C₁₋₄alkyl;

C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl,

—C(═O)—NH—C₁₋₄alkyl, —C(═O)—NH—C₁₋₄alkyl-C₃₋₆cycloalkyl, —C(═O)—OH, —NR^{11a}R^{11b}, and —NH—S(═O)₂—C₁₋₄alkyl; and

C₃₋₆cycloalkyl optionally substituted by one or two substituents each independently selected from the group consisting of —CN, —OH, —O—C₁₋₄alkyl, —C(═O)—NH—C₁₋₄alkyl,

—NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;

R⁸ represents —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, Het^{3a}, and Het^{6a};

Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;

Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b}; Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,

—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};

Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b}, and C₁₋₄alkyl;

Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;

Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl,

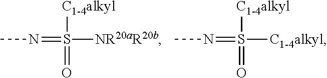

and

C₁₋₄alkyl substituted with one or two substituents each independently selected from the group consisting of Het^{3a}, Het^{6a}, Het^{6b}, and —NR^{9a}R^{9b};

Cy³ represents C₃₋₇cycloalkyl; wherein said C₃₋₇cycloalkyl is optionally substituted with one, two or three halo substituents;

R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵; Het⁷; —C₁₋₄alkyl-R¹⁶; —C(═O)—C₁₋₄alkyl-Het^{3a}; —C(═O)—R¹⁴;

C₃₋₆cycloalkyl substituted with one, two or three substituents selected from the group consisting of halo, —OH, —O—C₁₋₄alkyl, —NR^{11a}R^{11b}, and cyano; and

C₁₋₄alkyl substituted with one, two or three substituents selected from the group consisting of halo, —OH, —O—C₁₋₄alkyl, —NR^{11a}R^{11b}, and cyano;

R^{11a}, R^{11b}, R^{13a}, R^{13b}, R^{15a}, R^{15b}, R^{17a}, R^{17b}, R^{20a}, and R^{20b} are each independently selected from the group consisting of hydrogen and C₁₋₄alkyl;

R^{11c} and R^{11d} are each independently selected from the group consisting of hydrogen, C₁₋₆alkyl, and —C(═O)—C₁₋₄alkyl;

R^{10a} and R^{10b} are each independently selected from the group consisting of hydrogen, C₁₋₄alkyl, and C₃₋₆cycloalkyl;

R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;

R¹⁶ represents —C(═O)—NR^{17a}R^{17b}, —S(═O)₂—C₁₋₄alkyl, Het⁵, Het⁷, or Het⁸;

and the pharmaceutically acceptable salts and the solvates thereof.

The present invention relates in particular to compounds of Formula (I) as defined herein, and the tautomers and the stereoisomeric forms thereof, wherein

Q represents —CHR^{y}—, —O—, —C(═O)—, —NR^{q}—, or —CR^{y}═; the dotted line is an optional additional bond to form a double bond in case Q represents —CR^{y}═;

R^{1a} represents hydrogen; cyano; halo; Het; —C(═O)—NR^{xa}R^{xb}; —S(═O)₂—R¹⁸;

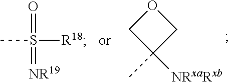

R¹⁸ represents C₁₋₆alkyl or C₃₋₆cycloalkyl;

R¹⁹ represents hydrogen or C₁₋₆alkyl;

Het represents a monocyclic 5- or 6-membered aromatic ring containing one, two or three nitrogen atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;

R^{xa} and R^{xb} are each independently selected from the group consisting of hydrogen; Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and NR^{11c}R^{11d};

or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;

or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;

R^{1b} represents hydrogen, F or Cl;

R² represents halo, C₃₋₆cycloalkyl, C₁₋₄alkyl, —O—C₁₋₄alkyl, cyano, or C₁₋₄alkyl substituted with one, two or three halo substituents;

R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;

Y and Y^{a} each independently represent a covalent bond or

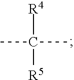

n1 and n2 are each independently selected from 1 and 2;

R^{y} represents hydrogen, —OH, C₁₋₄alkyl, —C₁₋₄alkyl-OH, or —C₁₋₄alkyl-O—C₁₋₄alkyl;

R^{q} represents hydrogen or C₁₋₄alkyl;

R⁵ represents hydrogen, C₁₋₄alkyl, or C₃₋₆cycloalkyl;

R³, R^{3a}, and R⁴ are each independently selected from the group consisting of Het¹; Het²; Cy²; C₁₋₆alkyl; and C₁₋₆alkyl substituted with one, two, three or four substituents each independently selected from the group consisting of —C(═O)—NR^{10a}R^{10b}, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;

R^{xc} represents Cy¹; Het⁵; —C₁₋₆alkyl-Cy¹; —C₁₋₆alkyl-Het³; —C₁₋₆alkyl-Het⁴;

or —C₁₋₆alkyl-phenyl;

R^{xd} represents hydrogen; C₁₋₄alkyl; or C₁₋₄alkyl substituted with one, two or three substituents selected from the group consisting of halo, —OH, —O—C₁₋₄alkyl, and cyano;

or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;

R^{8a} and R^{8b} are each independently selected from the group consisting of hydrogen; C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, and —C(═O)—NR^{10a}R^{10b};

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;

Het² represents C-linked pyrazolyl or triazolyl; which may be optionally substituted on one nitrogen atom with R^{6a};

R⁶ and R^{6a} are each independently selected from the group consisting of

Het³; Het⁴; —C(═O)—NH—Cy¹; —C(═O)—NH—R⁸; —S(═O)₂—C₁₋₄alkyl;

C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl, —C(═O)—OH, —NR^{11a}R^{11b}, and —NH—S(═O)₂—C₁₋₄alkyl; and

C₃₋₆cycloalkyl optionally substituted by one or two substituents each independently selected from the group consisting of —CN, —OH, —O—C₁₋₄alkyl, —C(═O)—NH—C₁₋₄alkyl,

—NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;

R⁸ represents —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, Het^{3a}, and Het^{6a};

Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;

wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;

Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};

Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo, —NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};

Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b}, and C₁₋₄alkyl;

Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;

Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl,

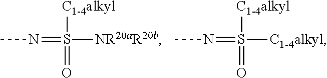

and

C₁₋₄alkyl substituted with one or two substituents each independently selected from the group consisting of Het^{3a}, Het^{6a}, Het^{6b}, and —NR^{9a}R^{9b};

Cy³ represents C₃₋₇cycloalkyl; wherein said C₃₋₇cycloalkyl is optionally substituted with one, two or three halo substituents;

R^{9a} and R^{9b} are each independently selected from the group consisting of hydrogen;

C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵; Het⁷; —C₁₋₄alkyl-R¹⁶; —C(═O)—C₁₋₄alkyl-Het^{3a}; —C(═O)—R¹⁴;

C₃₋₆cycloalkyl substituted with one, two or three substituents selected from the group consisting of halo, —OH, —O—C₁₋₄alkyl, —NR^{11a}R^{11b}, and cyano; and

C₁₋₄alkyl substituted with one, two or three substituents selected from the group consisting of halo, —OH, —O—C₁₋₄alkyl, —NR^{11a}R^{11b}, and cyano;

R^{11a}, R^{11b}, R^{13a}, R^{13b}, R^{15a}, R^{15b}, R^{17a}, R^{17b}, R^{20a} and R^{20b} are each independently selected from the group consisting of hydrogen and C₁₋₄alkyl;

R^{11c} and R^{11d} are each independently selected from the group consisting of hydrogen, C₁₋₆alkyl, and —C(═O)—C₁₋₄alkyl;

R^{10a} and R^{10b} are each independently selected from the group consisting of hydrogen, C₁₋₄alkyl, and C₃₋₆cycloalkyl;

R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;

R¹⁶ represents —C(═O)—NR^{17a}R^{17b}, —S(═O)₂—C₁₋₄alkyl, Het⁵, Het⁷, or Het⁸;

and the pharmaceutically acceptable salts and the solvates thereof.

The present invention relates in particular to compounds of Formula (I) as defined herein, and the tautomers and the stereoisomeric forms thereof, wherein

Q represents —CHR^{y}—, —O—, —C(═O)—, —NR^{q}—, or —CR^{y}═; the dotted line is an optional additional bond to form a double bond in case Q represents —CR^{y}═;

R^{1a} represents hydrogen; cyano; halo; Het; —C(═O)—NR^{xa}R^{xb}; —S(═O)₂—R¹⁸;

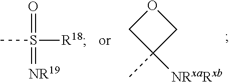

R¹⁸ represents C₁₋₆alkyl or C₃₋₆cycloalkyl;

R¹⁹ represents hydrogen or C₁₋₆alkyl;

Het represents a monocyclic 5- or 6-membered aromatic ring containing one, two or three nitrogen atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;

R^{xa} and R^{xb} are each independently selected from the group consisting of hydrogen; Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and NR^{11c}R^{11d};

or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;

or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;

R^{1b} represents hydrogen, F or Cl;

R² represents halo, C₃₋₆cycloalkyl, C₁₋₄alkyl, —O—C₁₋₄alkyl, cyano, or C₁₋₄alkyl substituted with one, two or three halo substituents;

R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;

Y and Y^{a} each independently represent a covalent bond or

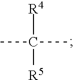

n1 and n2 are each independently selected from 1 and 2;

R^{y} represents hydrogen, —OH, C₁₋₄alkyl, —C₁₋₄alkyl-OH, or —C₁₋₄alkyl-O—C₁₋₄alkyl;

R^{q} represents hydrogen or C₁₋₄alkyl;

R⁵ represents hydrogen, C₁₋₄alkyl, or C₃₋₆cycloalkyl;

R³, R^{3a}, and R⁴ are each independently selected from the group consisting of Het¹; Het²; Cy²; C₁₋₆alkyl; and C₁₋₆alkyl substituted with one, two, three or four substituents each independently selected from the group consisting of —C(═O)—NR^{10a}R^{10b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;

R^{xc} represents Cy¹; Het⁵; —C₁₋₆alkyl-Cy¹; —C₁₋₆alkyl-Het³; —C₁₋₆alkyl-Het⁴;

or —C₁₋₆alkyl-phenyl;

R^{xd} represents hydrogen; C₁₋₄alkyl; or C₁₋₄alkyl substituted with one, two or three substituents selected from the group consisting of halo, —OH, —O—C₁₋₄alkyl, and cyano;

or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano; R^{8a} and R^{8b} are each independently selected from the group consisting of hydrogen; C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl;

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;

Het² represents C-linked pyrazolyl or triazolyl; which may be optionally substituted on one nitrogen atom with R^{6a};

R⁶ and R^{6a} are each independently selected from the group consisting of

Het³; Het⁴; —C(═O)—NH—Cy¹; —C(═O)—NH—R^{B}; —S(═O)₂—C₁₋₄alkyl;

C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl,

—C(═O)—NH—C₁₋₄alkyl, —C(═O)—NH—C₁₋₄alkyl-C₃₋₆cycloalkyl, —C(═O)—OH, —NR^{11a}R^{11b}, and —NH—S(═O)₂—C₁₋₄alkyl; and

C₃₋₆cycloalkyl optionally substituted by one or two substituents each independently selected from the group consisting of —CN, —OH, —O—C₁₋₄alkyl, —C(═O)—NH—C₁₋₄alkyl,

—NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;

R⁸ represents —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, Het^{3a}, and Het^{6a};

Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;

wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;

Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};

Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo, —NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};

Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b}, and C₁₋₄alkyl;

Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;

Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;

Cy² represents C₃₋₇cycloalkyl or a 5- to 12-membered saturated carbobicyclic system; wherein said C₃₋₇Cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, C₁₋₄alkyl C₁₋₄alkyl

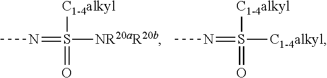

and

C₁₋₄alkyl substituted with one or two substituents each independently selected from the group consisting of Het^{3a}, Het^{6a}, Het^{6b}, and —NR^{9a}R^{9b};

Cy³ represents C₃₋₇cycloalkyl; wherein said C₃₋₇cycloalkyl is optionally substituted with one, two or three halo substituents;

R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵; Het⁷; —C₁₋₄alkyl-R¹⁶; —C(═O)—C₁₋₄alkyl-Het^{3a}; —C(═O)—R¹⁴;

C₃₋₆cycloalkyl substituted with one, two or three substituents selected from the group consisting of halo, —OH, —O—C₁₋₄alkyl, —NR^{11a}R^{11b}, and cyano; and C₁₋₄alkyl substituted with one, two or three substituents selected from the group consisting of halo, —OH, —O—C₁₋₄alkyl, —NR^{11a}R^{11b}, and cyano;

R^{11a}, R^{11b}, R^{13a}, R^{13b}, R^{15a}, R^{15b}, R^{17a}, R^{17b}, R^{20a}, and R^{20b} are each independently selected from the group consisting of hydrogen and C₁₋₄alkyl;

R^{11c} and R^{11d} are each independently selected from the group consisting of hydrogen, C₁₋₆alkyl, and —C(═O)—C₁₋₄alkyl;

R^{10a} and R^{10b} are each independently selected from the group consisting of hydrogen, C₁₋₄alkyl, and C₃₋₆cycloalkyl;

R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;

R¹⁶ represents —C(═O)—NR^{17a}R^{17b}, —S(═O)₂—C₁₋₄alkyl, Het⁵, Het⁷, or Het⁸;

and the pharmaceutically acceptable salts and the solvates thereof.

The present invention relates in particular to compounds of Formula (I) as defined herein, and the tautomers and the stereoisomeric forms thereof, wherein

Q represents —CHR^{y}—, —O—, —C(═O)—, —NR^{q}—, or —CR^{y}═; the dotted line is an optional additional bond to form a double bond in case Q represents —CR^{y}═;

R^{1a} represents hydrogen; cyano; halo; Het; —C(═O)—NR^{xa}R^{xb}; —S(═O)₂—R¹⁸;

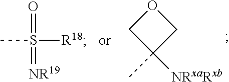

R¹⁸ represents C₁₋₆alkyl or C₃₋₆cycloalkyl;

R¹⁹ represents hydrogen or C₁₋₆alkyl;

Het represents a monocyclic 5- or 6-membered aromatic ring containing one, two or three nitrogen atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;

R^{xa} and R^{xb} are each independently selected from the group consisting of hydrogen; Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and NR^{11c}R^{11d};

or R^{ax} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;

or R^{ax} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;

R^{1b} represents hydrogen, F or Cl;

R² represents C₁₋₄alkyl; in particular R² represents methyl;

R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;

Y and Y^{a} each independently represent a covalent bond or

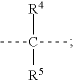

n1 and n2 are each independently selected from 1 and 2;

R^{y} represents hydrogen, —OH, C₁₋₄alkyl, —C₁₋₄alkyl-OH, or —C₁₋₄alkyl-O—C₁₋₄alkyl;

R^{q} represents hydrogen or C₁₋₄alkyl;

R⁵ represents hydrogen, C₁₋₄alkyl, or C₃₋₆cycloalkyl;

R³, R^{3a}, and R⁴ are each independently selected from the group consisting of Het¹; Het²; Cy²; C₁₋₆alkyl; and C₁₋₆alkyl substituted with one, two, three or four substituents each independently selected from the group consisting of —C(═O)—NR^{10a}R^{10b},

—NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;

R^{xc} represents Cy¹; Het⁵; —C₁₋₆alkyl-Cy¹; —C₁₋₆alkyl-Het³; —C₁₋₆alkyl-Het⁴; or —C₁₋₆alkyl-phenyl;

R^{xd} represents hydrogen; C₁₋₄alkyl; or C₁₋₄alkyl substituted with one, two or three substituents selected from the group consisting of halo, —OH, —O—C₁₋₄alkyl, and cyano;

or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl —S(═O)₂—C₁₋₄alkyl, and cyano;

R^{8a} and R^{8b} are each independently selected from the group consisting of hydrogen; C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, —C(═O)—NR^{10a}R^{10b}, and —NR^{10c}—C(═O)—C₁₋₄alkyl;

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;

Het² represents C-linked pyrazolyl or triazolyl; which may be optionally substituted on one nitrogen atom with R^{6a};

R⁶ and R^{6a} are each independently selected from the group consisting of

Het³; Het⁴; —C(═O)—NH—Cy¹; —C(═O)—NH—R^{B}; —S(═O)₂—C₁₋₄alkyl;

C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl, —C(═O)—OH, —NR^{11a}R^{11b}, and —NH—S(═O)₂—C₁₋₄alkyl; and

C₃₋₆cycloalkyl optionally substituted by one or two substituents each independently selected from the group consisting of —CN, —OH, —O—C₁₋₄alkyl, —C(═O)—NH—C₁₋₄alkyl,

—NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;

R⁸ represents —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, Het^{3a}, and Het^{6a};

Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl;

Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};

Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,

—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R_{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};

Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b}, and C₁₋₄alkyl;

Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;

Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl,

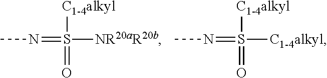

and

C₁₋₄alkyl substituted with one or two substituents each independently selected from the group consisting of Het^{3a}, Het^{6a}, Het^{6b}, and —NR^{9a}R^{9b};

Cy³ represents C₃₋₇cycloalkyl; wherein said C₃₋₇cycloalkyl is optionally substituted with one, two or three halo substituents;

R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵; Het⁷; —C₁₋₄alkyl-R¹⁶; —C(═O)—C₁₋₄alkyl-Het^{3a}; —C(═O)—R¹⁴;

C₃₋₆cycloalkyl substituted with one, two or three substituents selected from the group consisting of halo, —OH, —O—C₁₋₄alkyl, —NR^{11a}R^{11b}, and cyano; and C₁₋₄alkyl substituted with one, two or three substituents selected from the group consisting of halo, —OH, —O—C₁₋₄alkyl, —NR^{11a}R^{11b}, and cyano;

R^{11a}, R^{11b}, R^{13a}, R^{13b}, R^{15a}, R^{15b}, R^{17a}, R^{17b}, R^{20a}, and R^{20b} are each independently selected from the group consisting of hydrogen and C₁₋₄alkyl;

R^{11c} and R^{11d} are each independently selected from the group consisting of hydrogen, C₁₋₆alkyl, and —C(═O)—C₁₋₄alkyl;

R^{10a} and R^{10b} are each independently selected from the group consisting of hydrogen, C₁₋₄alkyl, and C₃₋₆cycloalkyl;

R¹⁴ represents Het^{8a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;

R¹⁶ represents —C(═O)—NR^{17a}R^{17b}, —S(═O)₂—C₁₋₄alkyl, Het⁵, Het⁷, or Het⁸; and the pharmaceutically acceptable salts and the solvates thereof.

The present invention relates in particular to compounds of Formula (I) as defined herein, and the tautomers and the stereoisomeric forms thereof, wherein

Q represents —CHR^{y}—, or —CR^{y}═; the dotted line is an optional additional bond to form a double bond in case Q represents —CR^{y}═;

R^{1a} represents hydrogen; halo; —C(═O)—NR^{xa}R^{xb}; or

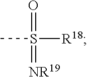

R¹⁸ represents C₁₋₆alkyl or C₃₋₆cycloalkyl;

R¹⁹ represents hydrogen or C₁₋₆alkyl;

R^{ax} and R^{xb} are each independently selected from the group consisting of hydrogen; Het³; and C₁₋₆alkyl; wherein optionally said C₁₋₆alkyl are substituted with 1, 2 or 3 substituents each independently selected from the group consisting of —OH, and —OC₁₋₄alkyl; or R^{ax} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, and —O—C₁₋₄alkyl;

or R^{ax} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, and —O—C₁₋₄alkyl;

R^{1b} represents F;

R² represents halo, C₁₋₄alkyl, or C₁₋₄alkyl substituted with one, two or three halo substituents;

R²¹ represents hydrogen;

Y represents a covalent bond or

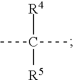

n1 and n2 are each independently selected from 1 and 2;

R^{y} represents hydrogen;

R⁵ represents hydrogen;

R³ and R⁴ are each independently selected from the group consisting of Het¹; Cy²;

C₁₋₆alkyl; and C₁₋₆alkyl substituted with one, two, three or four substituents each independently selected from the group consisting of —NR^{xc}R^{xd}, —NR^{8a}R^{8b}, —CF₃, —OH, Het¹, and Cy²;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;

R^{8a} and R^{8b} are each independently selected from the group consisting of C₁₋₆alkyl; and C₁₋₆alkyl substituted with one —O—C₁₋₄alkyl;

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶ and —C(═O)—R⁸; and wherein said heterocyclyl is optionally substituted on one or two carbon atoms with in total one or two substituents each independently selected from the group consisting of oxo and —NR^{9a}R^{9b};

R⁶ represents Het⁴; —C(═O)—NH—R⁸; —S(═O)₂—C₁₋₄alkyl; or C₁₋₆alkyl;

R⁸ represents —O—C₁₋₆alkyl, C₁₋₆alkyl; or C₁₋₆alkyl substituted with one, two or three substituents each independently selected from —O—C₁₋₄alkyl, and cyano;

Het³ represents a monocyclic C-linked 4- to 7-membered fully or partially saturated heterocyclyl containing one, two or three heteroatoms each independently selected from O, S, and N;

Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b};

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;

Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of R⁶, —C(═O)—Het^{6a}, Het^{6a}, Het^{6b}, —NR^{9a}R^{9b},

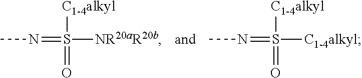

R^{9a} and R^{9b} are each independently selected from the group consisting of hydrogen;

C₁₋₄alkyl; —C(═O)—C₁₋₄alkyl; and —S(═O)₂—C₁₋₄alkyl;

R^{10a} and R^{10b} are each independently selected from the group consisting of hydrogen, C₁₋₄alkyl, and C₃₋₆cycloalkyl;

and the pharmaceutically acceptable salts and the solvates thereof.

The present invention relates in particular to compounds of Formula (I) as defined herein, and the tautomers and the stereoisomeric forms thereof, wherein

Q represents —CHR^{y}—, or —CR^{y}═; the dotted line is an optional additional bond to form a double bond in case Q represents —CR^{y}═;

R^{1a} represents hydrogen; halo; or —C(═O)—NR^{xa}R^{xb};

R^{xa} and R^{xb} are each independently selected from the group consisting of hydrogen and C₁₋₆alkyl;

R^{1b} represents F;

R² represents halo, C₁₋₄alkyl, or C₁₋₄alkyl substituted with one, two or three halo substituents;

R²¹ represents hydrogen;

Y represents a covalent bond or

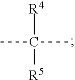

n1 and n2 are each independently selected from 1 and 2;

R^{y} represents hydrogen;

R⁵ represents hydrogen;

R³ and R⁴ are each independently selected from the group consisting of Het¹; Cy²; C₁₋₆alkyl; and C₁₋₆alkyl substituted with one, two, three or four substituents each independently selected from the group consisting of —NR^{xc}R^{xd}, —NR^{8a}R^{8b}, Het¹, and Cy²;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;

R^{8a} and R^{8b} are each independently selected from the group consisting of C₁₋₆alkyl; and C₁₋₆alkyl substituted with one —O—C₁₋₄alkyl;

Het¹ represents a monocyclic C-linked 4- to 7-membered fully or partially saturated heterocyclyl containing one, two or three heteroatoms each independently selected from O, S, and N; wherein said heterocyclyl is optionally substituted on one nitrogen with a substituent selected from the group consisting of R⁶ and —C(═O)—R⁸;

R⁶ represents Het⁴; —C(═O)—NH—R⁸; or —S(═O)₂—C₁₋₄alkyl;

R⁸ represents —O—C₁₋₆alkyl, C₁₋₆alkyl; or C₁₋₆alkyl substituted with one, two or three substituents each independently selected from —O—C₁₋₄alkyl, and cyano;

Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b};

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;

Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;

Cy² represents C₃₋₇cycloalkyl optionally substituted with one, two, three or four substituents each independently selected from the group consisting of R⁶, Het^{6a}, Het^{6b}, and —NR^{9a}R^{9b};

R^{9a} and R^{9b} are each independently selected from the group consisting of hydrogen; C₁₋₄alkyl; —C(═O)—C₁₋₄alkyl; and —S(═O)₂—C₁₋₄alkyl;

R^{10a} and R^{10b} are each independently selected from the group consisting of hydrogen and C₁₋₄alkyl;

and the pharmaceutically acceptable salts and the solvates thereof.

The present invention relates in particular to compounds of Formula (I) as defined herein, and the tautomers and the stereoisomeric forms thereof wherein

Q represents —CHR^{y}—;

R^{1a} represents —C(═O)—NR^{xa}R^{xb};

R^{a} and R^{b} represent C₁₋₆alkyl;

R^{1b} represents F;

R² represents halo or C₁₋₄alkyl;

R²¹ represents hydrogen;

Y represents a covalent bond or

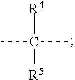

n1 and n2 are each independently selected from 1 and 2;

R^{y} represents hydrogen;

R⁵ represents hydrogen;

R³ is selected from the group consisting of Het¹; Cy²; C₁₋₆alkyl; and C₁₋₆alkyl substituted with one, two, three or four substituents each independently selected from the group consisting of —NR^{xc}R^{xd}, Het¹, and Cy²;

R⁴ represents C₁₋₆alkyl; in particular isopropyl;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;

Het¹ represents a monocyclic C-linked 4- to 7-membered fully or partially saturated heterocyclyl containing one, two or three heteroatoms each independently selected from O, S, and N; wherein said heterocyclyl is optionally substituted on one nitrogen with a substituent selected from the group consisting of R⁶ and —C(═O)—R⁸;

R⁶ represents Het⁴ or —C(═O)—NH—R^{B};

R⁸ represents C₁₋₆alkyl; or C₁₋₆alkyl substituted with one, two or three substituents each independently selected from —O—C₁₋₄alkyl, and cyano;

Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b};

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;

Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;

Cy² represents C₃₋₇cycloalkyl optionally substituted with one, two, three or four substituents each independently selected from the group consisting of R⁶, Het^{6a}, Het^{6b}, and —NR^{9a}R^{9b}; R^{9a} and R^{9b} are each independently selected from the group consisting of hydrogen; and —S(═O)₂—C₁₋₄alkyl;

R^{10a} and R^{10b} are each independently selected from the group consisting of hydrogen and C₁₋₄alkyl;

and the pharmaceutically acceptable salts and the solvates thereof.

The present invention relates in particular to compounds of Formula (I) as defined herein, and the tautomers and the stereoisomeric forms thereof, wherein

Q represents —CHR^{y}—;

R^{1a} represents —C(═O)—NR^{xa}R^{xb};

R^{xa} and R^{xb} represent C₁₋₆alkyl;

R^{1b} represents F;

R² represents C₁₋₄alkyl;

R²¹ represents hydrogen;

Y represents a covalent bond or

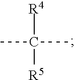

n1 and n2 are each independently selected from 1 and 2;

R^{y} represents hydrogen;

R⁵ represents hydrogen;

R³ is selected from the group consisting of Cy²; and C₁₋₆alkyl substituted with one, two, three or four substituents each independently selected from the group consisting of —NR^{xc}R^{xd}, Het¹, and Cy²;

R⁴ represents C₁₋₆alkyl; in particular isopropyl;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N; wherein said heterocyclyl is optionally substituted with one, two or three —(C═O)—C₁₋₄alkyl; Het¹ represents a monocyclic C-linked 4- to 7-membered fully or partially saturated heterocyclyl containing one, two or three heteroatoms each independently selected from O, S, and N; wherein said heterocyclyl is optionally substituted on one nitrogen with —C(═O)—R⁸;

R⁶ represents —C(═O)—NH—R⁸;

R⁸ represents C₁₋₆alkyl;

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;

Cy² represents C₃₋₇cycloalkyl optionally substituted with one, two, three or four substituents each independently selected from the group consisting of R⁶ and Het^{6a}; and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein

Q represents —CHR^{y}—;

R^{1a} represents —C(═O)—NR^{xa}R^{xb},

R^{xa} and R^{xb} are C₁₋₆alkyl optionally substituted with 1, 2 or 3 —OH;
R^{1b} represents F;
R² represents methyl;

R²¹ represents hydrogen or methyl;

Y represents a covalent bond;

n1 is 1;

n2 is selected from 1 and 2;

R^{y} represents hydrogen;

R³ is selected from C₁₋₈alkyl substituted with one, two, three or four substituents each independently selected from the group consisting of —NR^{xc}R^{xc}, Het¹ and Cy²;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R; and wherein said heterocyclyl is optionally substituted on one carbon atom with oxo;

R⁸ represents C₁₋₆alkyl; or C₁₋₆alkyl substituted with one, two or three substituents each independently selected from —OH, —O—C₁₋₄alkyl and cyano;

Het^{6a} represents a monocyclic N-linked 4- to 7-membered fully or partially saturated heterocyclyl containing one N-atom and optionally one or two additional heteroatoms each independently selected from O, S, and N, wherein said S-atom night be substituted to form S(═O) or S(═O)₂; wherein said heterocyclyl is optionally substituted on one nitrogen with —C(═O)—C₁₋₄alkyl;

Cy² represents C₃₋₇cycloalkyl optionally substituted with one Het^{6a};

and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein Q represents —CHR^{y}— or —CR^{y}═; the dotted line is an optional additional bond to form a double bond in case Q represents —CR^{y}═.

In an embodiment, the present invention relates to those compounds of Formula (I) and the pharmaceutically acceptable salts, and the solvates thereof, or any subgroup thereof as mentioned in any of the other embodiments, wherein Q represents —CHR^{y}—.

In an embodiment, the present invention relates to those compounds of Formula (I) and the pharmaceutically acceptable salts, and the solvates thereof, or any subgroup thereof as mentioned in any of the other embodiments, wherein

R^{1a} represents hydrogen; Het; —C(═O)—NR^{xa}R^{xb}; —S(═O)₂—R¹⁸;

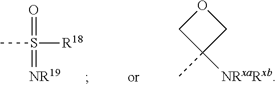

In an embodiment, the present invention relates to those compounds of Formula (I) and the pharmaceutically acceptable salts, and the solvates thereof, or any subgroup thereof as mentioned in any of the other embodiments, wherein

R^{1a} represents Het; —C(—O)—NR^{xa}R^{xb}; —S(═O)₂—R¹⁸;

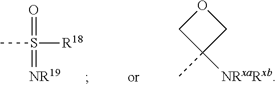

In an embodiment, the present invention relates to those compounds of Formula (I) and the pharmaceutically acceptable salts, and the solvates thereof, or any subgroup thereof as mentioned in any of the other embodiments, wherein

R^{1a} represents —C(═O)—NR^{xa}R^{xb}; —S(═O)₂—R¹⁸; or

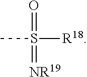

In an embodiment, the present invention relates to those compounds of Formula (I) and the pharmaceutically acceptable salts, and the solvates thereof, or any subgroup thereof as mentioned in any of the other embodiments, wherein R^{1a} represents —C(═O)—NR^{xa}R^{xb}; or

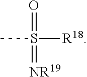

In an embodiment, the present invention relates to those compounds of Formula (I) and the pharmaceutically acceptable salts, and the solvates thereof, or any subgroup thereof as mentioned in any of the other embodiments, wherein

R^{1a} represents —C(═O)—NR^{xa}R^{xb}.

In an embodiment, the present invention relates to those compounds of Formula (I) and the pharmaceutically acceptable salts, and the solvates thereof, or any subgroup thereof as mentioned in any of the other embodiments, wherein R¹⁸ represents C₁₋₆alkyl.

In an embodiment, the present invention relates to those compounds of Formula (I) and the pharmaceutically acceptable salts, and the solvates thereof, or any subgroup thereof as mentioned in any of the other embodiments, wherein R^{xa} and R^{xb} represent hydrogen or C₁₋₆alkyl.

In an embodiment, the present invention relates to those compounds of Formula (I) and the pharmaceutically acceptable salts, and the solvates thereof, or any subgroup thereof as mentioned in any of the other embodiments, wherein R^{xa} and R^{xb} are each independently selected from the group consisting of hydrogen; Het³; and C₁₋₆alkyl; wherein optionally said C₁₋₈alkyl are substituted with 1, 2 or 3 substituents each independently selected from the group consisting of —OH, and —OC₁₋₄alkyl;

or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, and —O—C₁₋₄alkyl;

or R^{ax} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, and —O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R^{xa} and R^{xb} are each independently selected from the group consisting of hydrogen; Het³; and C₁₋₆alkyl; wherein optionally said C₁₋₆alkyl are substituted with 1, 2 or 3 substituents each independently selected from the group consisting of —OH, and —OC₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R^{xa} and R^{xb} represent C₁₋₆alkyl.

In an embodiment, the present invention relates to those compounds of Formula (I) and the pharmaceutically acceptable salts, and the solvates thereof, or any subgroup thereof as mentioned in any of the other embodiments, wherein R^{xa} and R^{xb} are taken together.

In an embodiment, the present invention relates to those compounds of Formula (I) and the pharmaceutically acceptable salts, and the solvates thereof, or any subgroup thereof as mentioned in any of the other embodiments, wherein R^{xa} and R^{xb} are not taken together.

In an embodiment, the present invention relates to those compounds of Formula (I) and the pharmaceutically acceptable salts, and the solvates thereof, or any subgroup thereof as mentioned in any of the other embodiments, wherein R^{1b} represents F or Cl.

In an embodiment, the present invention relates to those compounds of Formula (I) and the pharmaceutically acceptable salts, and the solvates thereof, or any subgroup thereof as mentioned in any of the other embodiments, wherein R^{1b} represents F.

In an embodiment, the present invention relates to those compounds of Formula (I) and the pharmaceutically acceptable salts, and the solvates thereof, or any subgroup thereof as mentioned in any of the other embodiments, wherein R² represents halo, C₁₋₄alkyl, or C₁₋₄alkyl substituted with one, two or three halo substituents.

In an embodiment, the present invention relates to those compounds of Formula (I) and the pharmaceutically acceptable salts, and the solvates thereof, or any subgroup thereof as mentioned in any of the other embodiments, wherein R² represents halo or C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R² represents C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R² represents methyl.

In an embodiment, the present invention relates to those compounds of Formula (I) and the pharmaceutically acceptable salts, and the solvates thereof, or any subgroup thereof as mentioned in any of the other embodiments, wherein R² represents methyl; and R^{1a} represents —C(═O)—NR^{xa}R^{xb}.

In an embodiment, the present invention relates to those compounds of Formula (I) and the pharmaceutically acceptable salts, and the solvates thereof, or any subgroup thereof as mentioned in any of the other embodiments, wherein Y and Y^{a} represent a covalent bond.

In an embodiment, the present invention relates to those compounds of Formula (I) and the pharmaceutically acceptable salts, and the solvates thereof, or any subgroup thereof as mentioned in any of the other embodiments, wherein R²¹ represents hydrogen.

In an embodiment, the present invention relates to those compounds of Formula (I) and the pharmaceutically acceptable salts, and the solvates thereof, or any subgroup thereof as mentioned in any of the other embodiments, wherein R²¹ represents hydrogen or methyl.

In an embodiment, the present invention relates to those compounds of Formula (I) and the pharmaceutically acceptable salts, and the solvates thereof, or any subgroup thereof as mentioned in any of the other embodiments, wherein

R²¹ represents hydrogen; and

Y represents a covalent bond.

In an embodiment, the present invention relates to those compounds of Formula (I) and the pharmaceutically acceptable salts, and the solvates thereof, or any subgroup thereof as mentioned in any of the other embodiments, wherein

R²¹ represents hydrogen or methyl; and

Y represents a covalent bond.

In an embodiment, the present invention relates to those compounds of Formula (I) and the pharmaceutically acceptable salts, and the solvates thereof, or any subgroup thereof as mentioned in any of the other embodiments, wherein R²¹ represents —Y^{a}—R^{3a}.

In an embodiment, the present invention relates to those compounds of Formula (I) and the pharmaceutically acceptable salts, and the solvates thereof, or any subgroup thereof as mentioned in any of the other embodiments, wherein R²¹ represents hydrogen, C₁₋₆alkyl, C₃₋₆cycloalkyl, or C₁₋₆alkyl substituted with 1 substituent selected from the group consisting of halo, —OH, —O—C₁₋₄alkyl, —C(═O)—NR^{10a}R^{10b}, —NR^{10c}—C(═O)—C₁₋₄alkyl, and —S(═O)₂—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R²¹ represents C₁₋₆alkyl, C₃₋₆cycloalkyl, or C₁₋₆alkyl substituted with 1 substituent selected from the group consisting of halo, —OH, —O—C₁₋₄alkyl, —C(═O)—NR^{10a}R^{10b}, —NR^{10c}—C(═O)—C₁₋₄alkyl, and —S(═O)₂—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R^{10c} is selected from the group consisting of hydrogen, C₁₋₄alkyl, and C₃₋₆cycloalkyl.

In an embodiment, the present invention relates to those compounds of Formula (I) and the pharmaceutically acceptable salts, and the solvates thereof, or any subgroup thereof as mentioned in any of the other embodiments, wherein

R³, R^{3a}, and R⁴ are each independently selected from the group consisting of Het¹; Het²; Cy²; C₁₋₆alkyl; and C₁₋₆alkyl substituted with one, two, three or four substituents each independently selected from the group consisting of —C(═O)—NR^{10a}R^{10b}, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;

R^{8a} and R^{8b} are each independently selected from the group consisting of hydrogen; C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl, and —C(═O)—NR^{10a}R^{10b}.

In an embodiment, the present invention relates to those compounds of Formula (I) and the pharmaceutically acceptable salts, and the solvates thereof, or any subgroup thereof as mentioned in any of the other embodiments, wherein

R³, R^{3a}, and R⁴ are not C₁₋₆alkyl substituted with —NR^{10b}—C(═O)—C₁₋₄alkyl;

R^{8a} and R^{8b} are not C₁₋₆alkyl substituted with —NR^{10c}—C(═O)—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein Y represents a covalent bond.

In an embodiment, the present invention relates to those compounds of Formula (I) and the pharmaceutically acceptable salts, and the solvates thereof, or any subgroup thereof as mentioned in any of the other embodiments, wherein Y^{a} represents a covalent bond.

In an embodiment, the present invention relates to those compounds of Formula (I) and the pharmaceutically acceptable salts, and the solvates thereof, or any subgroup thereof as mentioned in any of the other embodiments, wherein Y represents

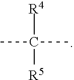

In an embodiment, the present invention relates to those compounds of Formula (I) and the pharmaceutically acceptable salts, and the solvates thereof, or any subgroup thereof as mentioned in any of the other embodiments, wherein Y^{a} represents

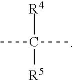

In an embodiment, the present invention relates to those compounds of Formula (I) and the pharmaceutically acceptable salts, and the solvates thereof, or any subgroup thereof as mentioned in any of the other embodiments, wherein n1 represents 1, and n2 represents 2.

In an embodiment, the present invention relates to those compounds of Formula (I) and the pharmaceutically acceptable salts, and the solvates thereof, or any subgroup thereof as mentioned in any of the other embodiments, wherein

R⁴ represents C₁₋₆alkyl; oxetanyl; tetrahydropyranyl;

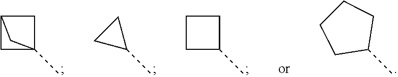

In an embodiment, the present invention relates to those compounds of Formula (I) and the pharmaceutically acceptable salts, and the solvates thereof, or any subgroup thereof as mentioned in any of the other embodiments, wherein R^{y} represents hydrogen.

In an embodiment, the present invention relates to those compounds of Formula (I) and the pharmaceutically acceptable salts, and the solvates thereof, or any subgroup thereof as mentioned in any of the other embodiments, wherein

R⁴ represents C₁₋₆alkyl; oxetanyl; tetrahydropyranyl;

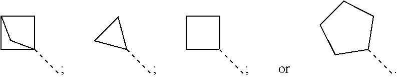

In an embodiment, the present invention relates to those compounds of Formula (I) and the pharmaceutically acceptable salts, and the solvates thereof, or any subgroup thereof as mentioned in any of the other embodiments, wherein R⁴ represents C₁₋₆alkyl.

In an embodiment, the present invention relates to those compounds of Formula (I) and the pharmaceutically acceptable salts, and the solvates thereof, or any subgroup thereof as mentioned in any of the other embodiments, wherein R⁴ represents isopropyl.

In an embodiment, the present invention relates to those compounds of Formula (I) and the pharmaceutically acceptable salts, and the solvates thereof, or any subgroup thereof as mentioned in any of the other embodiments, wherein R⁴ represents C₁₋₈alkyl.

In an embodiment, the present invention relates to those compounds of Formula (I) and the pharmaceutically acceptable salts, and the solvates thereof, or any subgroup thereof as mentioned in any of the other embodiments, wherein R⁴ represents C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R⁵ represents hydrogen.

In an embodiment, the present invention relates to those compounds of Formula (I) and the pharmaceutically acceptable salts, and the solvates thereof, or any subgroup thereof as mentioned in any of the other embodiments, wherein

R³ and R⁴ are each independently selected from the group consisting of Het¹; Cy²; C₁₋₆alkyl; and C₁₋₆alkyl substituted with one, two, three or four substituents each independently selected from the group consisting of —NR^{xc}R^{xd}, —NR^{8a}R^{8b}, Het¹, and Cy².

In an embodiment, the present invention relates to those compounds of Formula (I) and the pharmaceutically acceptable salts, and the solvates thereof, or any subgroup thereof as mentioned in any of the other embodiments, wherein

R³ is selected from the group consisting of Het¹; Cy²; C₁₋₆alkyl; and C₁₋₆alkyl substituted with one, two, three or four substituents each independently selected from the group consisting of —NR^{xc}R^{xd}, Het¹, and Cy²; and

R⁴ represents C₁₋₆alkyl; in particular isopropyl.

In an embodiment, the present invention relates to those compounds of Formula (I) and the pharmaceutically acceptable salts, and the solvates thereof, or any subgroup thereof as mentioned in any of the other embodiments, wherein

R³ is selected from the group consisting of Cy²; and C₁₋₆alkyl substituted with one, two, three or four substituents each independently selected from the group consisting of —NR^{xc}R^{xd}, Het¹, and Cy²; and

R⁴ represents C₁₋₆alkyl; in particular isopropyl.

In an embodiment, the present invention relates to those compounds of Formula (I) and the pharmaceutically acceptable salts, and the solvates thereof, or any subgroup thereof as mentioned in any of the other embodiments, wherein

R³ is selected from the group consisting of Het¹; Cy²; C₁₋₆alkyl; and C₁₋₆alkyl substituted with one, two, three or four substituents each independently selected from the group consisting of —NR^{xc}R^{xd}, Het¹, and Cy².

In an embodiment, the present invention relates to those compounds of Formula (I) and the pharmaceutically acceptable salts, and the solvates thereof, or any subgroup thereof as mentioned in any of the other embodiments, wherein

R³ is selected from the group consisting of Cy²; and C₁₋₆alkyl substituted with one, two, three or four substituents each independently selected from the group consisting of —NR^{xc}R^{xd}, Het¹, and Cy².

In an embodiment, the present invention relates to those compounds of Formula (I) and the pharmaceutically acceptable salts, and the solvates thereof, or any subgroup thereof as mentioned in any of the other embodiments, wherein Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —NR^{9a}R^{9b}, and —OH.

In an embodiment, the present invention relates to those compounds of Formula (I) and the pharmaceutically acceptable salts, and the solvates thereof, or any subgroup thereof as mentioned in any of the other embodiments, wherein

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R^{xc} and R^{xd} are taken together.

In an embodiment, the present invention relates to those compounds of Formula (I) and the pharmaceutically acceptable salts, and the solvates thereof, or any subgroup thereof as mentioned in any of the other embodiments, wherein R^{xc} and R^{xd} are not taken together.

In an embodiment, the present invention relates to those compounds of Formula (I) and the pharmaceutically acceptable salts, and the solvates thereof, or any subgroup thereof as mentioned in any of the other embodiments, wherein fully or partially saturated heterocyclyl groups are limited to fully saturated heterocyclycl groups.

In an embodiment, the present invention relates to those compounds of Formula (I) and the pharmaceutically acceptable salts, and the solvates thereof, or any subgroup thereof as mentioned in any of the other embodiments, wherein

Q represents —CHR^{y}—;

R^{1a} represents —C(═O)—NR^{xa}R^{xb}.

R^{1b} represents F;

R² represents methyl.

In an embodiment, the present invention relates to those compounds of Formula (I) and the pharmaceutically acceptable salts, and the solvates thereof, or any subgroup thereof as mentioned in any of the other embodiments, wherein

R^{8a} and R^{8b} are each independently selected from the group consisting of C₁₋₆alkyl; and C₁₋₆alkyl substituted with one —O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶ and —C(═O)—R⁸; and wherein said heterocyclyl is optionally substituted on one or two carbon atoms with in total one or two substituents each independently selected from the group consisting of oxo and —NR^{9a}R^{9b}.

In an embodiment, the present invention relates to those compounds of Formula (I) and the pharmaceutically acceptable salts, and the solvates thereof, or any subgroup thereof as mentioned in any of the other embodiments, wherein R⁶ represents Het⁴ or —C(═O)—NH—R.

In an embodiment, the present invention relates to those compounds of Formula (I) and the pharmaceutically acceptable salts, and the solvates thereof, or any subgroup thereof as mentioned in any of the other embodiments, wherein R⁸ represents C₁₋₆alkyl; or C₁₋₆alkyl substituted with one, two or three substituents each independently selected from —O—C₁₋₄alkyl, and cyano.

In an embodiment, the present invention relates to those compounds of Formula (I) and the pharmaceutically acceptable salts, and the solvates thereof, or any subgroup thereof as mentioned in any of the other embodiments, wherein R⁸ represents C₁₋₆alkyl.

In an embodiment, the present invention relates to those compounds of Formula (I) and the pharmaceutically acceptable salts, and the solvates thereof, or any subgroup thereof as mentioned in any of the other embodiments, wherein R⁸ represents methyl.

In an embodiment, the present invention relates to those compounds of Formula (I) and the pharmaceutically acceptable salts, and the solvates thereof, or any subgroup thereof as mentioned in any of the other embodiments, wherein

Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b}.

In an embodiment, the present invention relates to those compounds of Formula (I) and the pharmaceutically acceptable salts, and the solvates thereof, or any subgroup thereof as mentioned in any of the other embodiments, wherein

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of R⁶, —C(═O)—Het^{6a}, Het^{6a}, Het^{6b}, —NR^{9a}R^{9b},

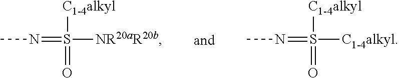

In an embodiment, the present invention relates to those compounds of Formula (I) and the pharmaceutically acceptable salts, and the solvates thereof, or any subgroup thereof as mentioned in any of the other embodiments, wherein

Cy² represents C₃₋₇cycloalkyl optionally substituted with one, two, three or four substituents each independently selected from the group consisting of R⁶, Het^{6a}, Het^{6b}, —NR^{9a}R^{9b},

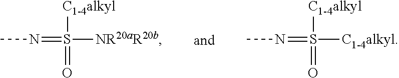

In an embodiment, the present invention relates to those compounds of Formula (I) and the pharmaceutically acceptable salts, and the solvates thereof, or any subgroup thereof as mentioned in any of the other embodiments, wherein

Cy² represents C₃₋₇cycloalkyl optionally substituted with one, two, three or four substituents each independently selected from the group consisting of R⁶, Het^{6a}, Het^{6b}, and —NR^{9a}R^{9b}.

In an embodiment, the present invention relates to those compounds of Formula (I) and the pharmaceutically acceptable salts, and the solvates thereof, or any subgroup thereof as mentioned in any of the other embodiments, wherein R^{9a} and R^{9b} are each independently selected from the group consisting of hydrogen; and —S(═O)₂—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R^{10a} and R^{10b} are each independently selected from the group consisting of hydrogen and C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein when R^{xa} and R^{xb} are taken together to form a monocyclic heterocyclyl they represent 1-pyrrolidinyl or 1-piperidinyl, each optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein when R^{xa} and R^{xb} are taken together to form a bicyclic heterocyclyl they represent

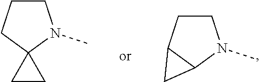

each optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein when R^{xc} and R^{xd} are taken together to form a monocyclic heterocyclyl they represent 1-pyrrolidinyl, 1-piperidinyl, or 1-piperazinyl, each optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein when R^{xc} and R^{xd} are taken together to form a bicyclic heterocyclyl they represent

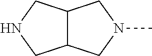

optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein Het¹ represents

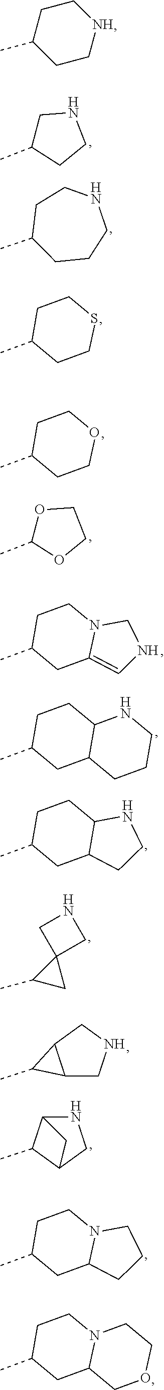

optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein Het¹ represents

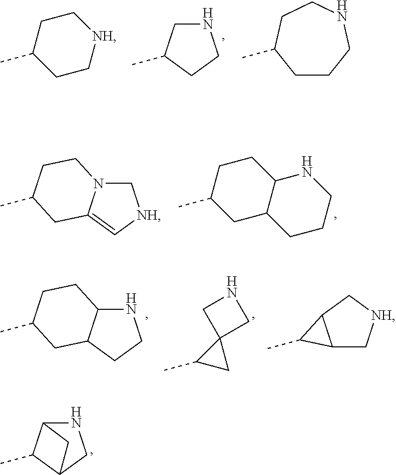

optionally substituted on a nitrogen atom with —C(═O)—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein Het¹ represents

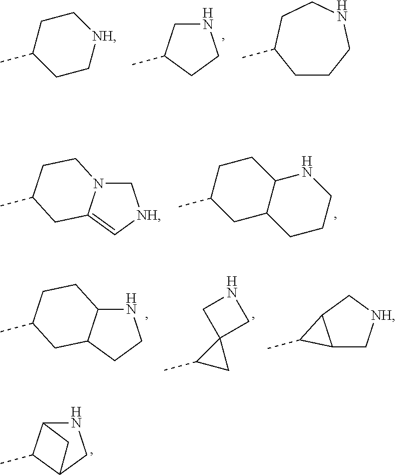

substituted on a nitrogen atom with —C(═O)—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a fused or spiro bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH.

In an embodiment, the present invention relates to those compounds of Formula (I) and the pharmaceutically acceptable salts, and the solvates thereof, or any subgroup thereof as mentioned in any of the other embodiments, wherein

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a fused or spiro bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶ and —C(═O)—R⁸; and wherein said heterocyclyl is optionally substituted on one or two carbon atoms with in total one or two substituents each independently selected from the group consisting of oxo and —NR^{9a}R^{9b}.

In an embodiment, the present invention relates to those compounds of Formula (I) and the pharmaceutically acceptable salts, and the solvates thereof, or any subgroup thereof as mentioned in any of the other embodiments, wherein

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH.

In an embodiment, the present invention relates to those compounds of Formula (I) and the pharmaceutically acceptable salts, and the solvates thereof, or any subgroup thereof as mentioned in any of the other embodiments, wherein

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶ and —C(═O)—R⁸; and wherein said heterocyclyl is optionally substituted on one or two carbon atoms with in total one or two substituents each independently selected from the group consisting of oxo and —NR^{9a}R^{9b}.

In an embodiment, the present invention relates to those compounds of Formula (I) and the pharmaceutically acceptable salts, and the solvates thereof, or any subgroup thereof as mentioned in any of the other embodiments, wherein Het³ represents

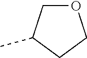

optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein Het⁴ represents C-linked pyrazinyl optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein Het^{6a} represents

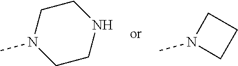

optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein Het^{6a} represents

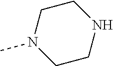

optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein Het^{6a} represents

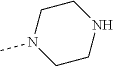

substituted on a nitrogen atom with —C(═O)—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein Het^{6b} represents

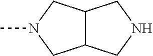

optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein Het^{6b} represents

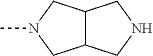

substituted on a nitrogen atom with —C(═O)—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein Cy² represents C₃₋₇cycloalkyl,

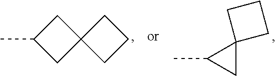

optionally substituted as defined in any of the other embodiments.

In an embodiment, the present invention relates to those compounds of Formula (I) and the pharmaceutically acceptable salts, and the solvates thereof, or any subgroup thereof as mentioned in any of the other embodiments, wherein C₁₋₈alkyl is limited to C₁₋₆alkyl, in particular wherein C₁₋₈alkyl is limited to C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein —Y—R³ is attached to the nitrogen atom of the ring.

In an embodiment, the present invention relates to those compounds of Formula (I) and the pharmaceutically acceptable salts, and the solvates thereof, or any subgroup thereof as mentioned in any of the other embodiments, wherein

R²¹ is hydrogen, and wherein —Y—R³ is attached to the nitrogen atom of the ring.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-x):

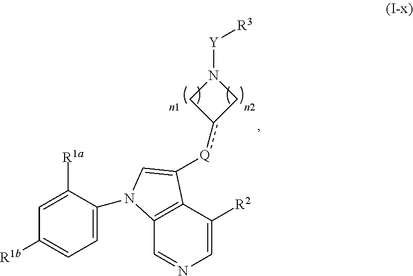

wherein the variables are as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-x1):

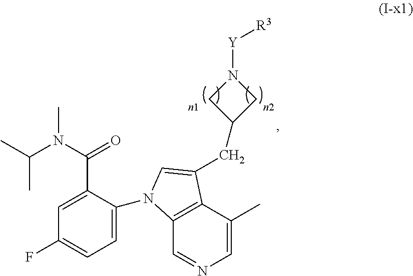

wherein the variables are as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-x2):

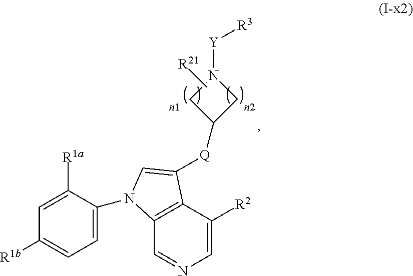

wherein Q represents —CHR^{y}—, —O—, —C(═O)— or —NR^{q}—; and wherein the other variables are as defined for the compounds of Formula (I) or any subgroup thereof as mentioned in any of the other embodiments.

The present invention relates in particular to compounds of Formula (T-x2) as defined herein, and the tautomers and the stereoisomeric forms thereof, wherein

Q represents —CHR^{y}—, —O—, —C(═O)— or —NR^{q}—;

R^{1a} represents hydrogen; cyano; halo; Het; —C(═O)—NR^{xa}R^{xb}; —S(═O)₂—R¹⁸; —C(═O)—O—C₁₋₄alkyl-NR^{22a}R^{22b}; —C(═O)—O—C₁₋₄alkyl;

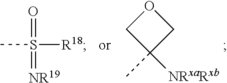

R¹⁸ represents C₁₋₆alkyl or C₃₋₆cycloalkyl;

R¹⁹ represents hydrogen or C₁₋₆alkyl;

or R¹⁸ and R¹⁹ are taken together to form —(CH₂)₃—, —(CH₂)₄— or —(CH₂)—;

Het represents a monocyclic 5- or 6-membered aromatic ring containing one, two or three O-, S- or N-atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;

R^{xa} and R^{xb} are each independently selected from the group consisting of hydrogen; Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, —C₁₋₄alkyl-OH, halo, CF₃, C₃₋₆cycloalkyl, Het³, and NR^{11c}R^{11d};

or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents selected from the group consisting of halo and OR²³;

or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents each independently selected from the group consisting of halo and OR²³;

R²³ represents hydrogen or C₁₋₄alkyl optionally substituted with one, two or three halo;

R^{1b} represents hydrogen, F, Cl, or —O—C₁₋₄alkyl;

R² represents halo, C₃₋₆cycloalkyl, C₁₋₄alkyl, —O—C₁₋₄alkyl, cyano, or C₁₋₄alkyl substituted with one, two or three halo substituents;

R²¹ represents hydrogen or —Y^{a}—R^{3a};

Y and Y^{a} each independently represent a covalent bond or

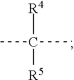

n1 is selected from 1 and 2;

n2 is selected from 1, 2, 3 and 4;

R^{y} represents hydrogen, —OH, C₁₋₄alkyl, —C₁₋₄alkyl-OH, or —C₁₋₄alkyl-O—C₁₋₄alkyl;

R^{q} represents hydrogen or C₁₋₄alkyl;

R⁵ represents hydrogen. C₁₋₄alkyl, or C₃₋₆cycloalkyl;

R³, R^{3a}, and R⁴ are each independently selected from the group consisting of Het¹; Het²; Cy²; C₁₋₈alkyl; and C₁₋₈alkyl substituted with one, two, three or four substituents each independently selected from the group consisting of —C(═O)—NR^{10a}R^{10b}. —C(═O)—Het^{6a}, —C(═O)—Het^{6b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², Ar¹, and Cy²;

R^{xc} represents Cy¹; Het⁵; —C₁₋₆alkyl-Cy¹; —C₁₋₆alkyl-Het³; —C₁₋₆alkyl-Het⁴; or —C₁₋₆alkyl-phenyl;

R^{xd} represents hydrogen; C₁₋₄alkyl; or C₁₋₄alkyl substituted with one, two or three substituents selected from the group consisting of halo, —OH, —O—C₁₋₄alkyl, and cyano;

or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;

or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;

R^{8a} and R^{8b} are each independently selected from the group consisting of hydrogen; C₁₋₆alkyl; —(C═O)—C₁₋₄alkyl; and C₁₋₆alkyl substituted with one, two or three substituents each independently selected from the group consisting of —OH, cyano, halo, —S(═O)₂—C₁₋₄alkyl, —O—C₁₋₄alkyl,

—C(═O)—NR^{10a}R^{10b}, and —NR^{10c}—C(═O)—C₁₋₄alkyl;

Ar¹ represents phenyl optionally substituted with one, two or three substituents each independently selected from the group consisting of C₁₋₄alkyl, halo, —O—C₁₋₄alkyl, —CF₃, —OH, —S(═O)₂—C₁₋₄alkyl, and —C(═O)—NR^{10a}R^{10b};

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;

Het² represents C-linked pyrazolyl, 1,2,4-oxadiazolyl, pyridazinyl or triazolyl; which may be optionally substituted on one nitrogen atom with R^{6a},

R⁶ and R^{6a} are each independently selected from the group consisting of Het³; Het⁴; —C(═O)—NH—Cy¹; —C(═O)—NH—R⁸; —C(═O)—Het^{6a}, —C(═O)—NR_{10d}R^{10e}; —C(═O)—O—C₁₋₄alkyl; —S(═O)₂—C₁₋₄alkyl;

C₁₋₆alkyl optionally substituted with one or two substituents each independently selected from the group consisting of Het³, Het⁴, Het^{6a}, Het^{6b}. Cy¹, —CN, —OH, —O—C₁₋₄alkyl, —C(═O)—NH—C₁₋₄alkyl, —C(═O)—N(C₁₋₄alkyl)₂, —C(O)—NH—C₁₋₄alkyl-C₃₋₆cycloalkyl, —C(═O)—OH, —NR^{11a}R^{11b}, and —NH—S(═O)₂—C₁₋₄alkyl; and

C₃₋₆cycloalkyl optionally substituted by one or two substituents each independently selected from the group consisting of —CN, —OH, —O—C₁₋₄alkyl, —C(═O)—NH—C₁₋₄alkyl, —C(═O)—N(C₁₋₄alkyl)₂, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;

R⁸ represents hydrogen, —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, —S(═O)₂—C₁₋₄alkyl, Het^{3a}, and Het^{6a};

Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl or —(C═O)—C₁₋₄alkyl;

Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}; —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b}:

Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,

—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b}.

Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl, —C(═O)—C₁₋₄alkyl-NR^{11a}R^{11b}, and C₁₋₄alkyl;

Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —Oil, halo, and C₁₋₄alkyl;

Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;

Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, —O—C₁₋₄alkyl, cyano,

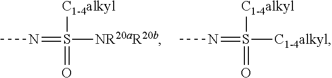

and

C₁₋₄alkyl substituted with one or two substituents each independently selected from the group consisting of Het^{3a}, Het^{6a}, Het^{6b}, and —NR^{9a}R^{9b};

Cy³ represents C₃₋₇cycloalkyl; wherein said C₃₋₇cycloalkyl is optionally substituted with one, two or three halo substituents;

R^{9a} and R^{9b} are each independently selected from the group consisting of hydrogen; C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵; Het⁷; —C₁₋₄alkyl-R¹⁶; —C(═O)—C₁₋₄alkyl-Het^{3a}; —C(═O)—R¹⁴;

C₃₋₆cycloalkyl substituted with one, two or three substituents selected from the group consisting of halo, —OH, —O—C₁₋₄alkyl, —NR^{11a}R^{11b}, and cyano; and

C₁₋₄alkyl substituted with one, two or three substituents selected from the group consisting of halo, —OH, —O—C₁₋₄alkyl, —NR^{11a}R^{11b}, and cyano;

R^{11a}, R^{11b}, R^{13a}, R^{13b}, R^{15a}, R^{15b}, R^{17a}, R^{17b}, R^{20a}, R^{20b}, R^{22a}, and R^{22b} are each independently selected from the group consisting of hydrogen and C₁₋₄alkyl;

R^{11c} and R^{11d} are each independently selected from the group consisting of hydrogen, C₁₋₆alkyl, and —C(═O)—C₁₋₄alkyl;

R^{10a}, R^{10b} and R^{10C} are each independently selected from the group consisting of hydrogen, C₁₋₄alkyl, and C₃₋₆cycloalkyl;

R^{10d} and R^{10e} are each independently selected from the group consisting of C₁₋₄alkyl, —O—C₁₋₄alkyl and C₃₋₆cycloalkyl;

R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;

R¹⁶ represents —C(═)—NR^{17a}R^{17b}, —S(═O)₂—C₁₋₄alkyl, Het⁵, Het⁷, or Het⁸;

and the pharmaceutically acceptable salts and the solvates thereof.

The present invention relates in particular to compounds of Formula (I-x2) as defined herein, and the tautomers and the stereoisomeric forms thereof, wherein

Q represents —CHR^{y}—;

R^{1a} represents —C(═O)—NR^{xa}R^{xb};

R^{xa} and R^{xb} are C₁₋₆alkyl optionally substituted with 1, 2 or 3 —OH;

R^{1b} represents F;

R² represents methyl;

R²¹ represents hydrogen or methyl;

Y represents a covalent bond or

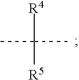

R⁵ represents hydrogen;

n1 is 1;

n2 is selected from 1 and 2;

R^{y} represents hydrogen;

R³ and R⁴ are each independently selected from Het¹, Cy², and C₁₋₈alkyl substituted with one, two, three or four substituents each independently selected from the group consisting of —NR^{xc}R^{xd}, Het¹ and Cy;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸; and wherein said heterocyclyl is optionally substituted on one carbon atom with oxo;
R⁸ represents C₁₋₆alkyl; or C₁₋₆alkyl substituted with one, two or three substituents each independently selected from —OH, —O—C₁₋₄alkyl and cyano;

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;

Cy² represents C₃₋₇cycloalkyl optionally substituted with one Het^{6a};

and the pharmaceutically acceptable salts and the solvates thereof.

The present invention relates in particular to compounds of Formula (I-x2) as defined herein, and the tautomers and the stereoisomeric forms thereof, wherein

Q represents —CHR^{y}—;

R^{1a} represents —C(═O)—NR^{xa}R^{xb};

R^{xa} and R^{xb} are C₁₋₆alkyl optionally substituted with 1, 2 or 3 —OH;

R^{1b} represents F;

R² represents methyl;

R²¹ represents hydrogen or methyl;

Y represents a covalent bond or

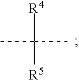

R⁵ represents hydrogen;

n1 is 1;

n2 is selected from 1 and 2;

R^{y} represents hydrogen;

R³ and R⁴ are each independently selected from Het¹, Cy², and C₁₋₆alkyl substituted with one, two, three or four substituents each independently selected from the group consisting of —NR^{xc}R^{xd}, Het¹ and Cy²;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸; and wherein said heterocyclyl is optionally substituted on one carbon atom with oxo;

R⁸ represents C₁₋₆alkyl; or C₁₋₆alkyl substituted with one, two or three substituents each independently selected from —OH, —O—C₁₋₄alkyl and cyano;

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;

Cy² represents C₃₋₇cycloalkyl optionally substituted with one Het^{6a}; and the pharmaceutically acceptable salts and the solvates thereof.

The present invention relates in particular to compounds of Formula (I-x2) as defined herein, and the tautomers and the stereoisomeric forms thereof, wherein

Q represents —CHR^{y}—;

R^{1a} represents —C(═O)—NR^{xa}R^{xb};

R^{xa} and R^{xb} are C₁₋₆alkyl optionally substituted with 1, 2 or 3 —OH;

R^{1b} represents F;
R² represents methyl;

R²¹ represents hydrogen or methyl;

Y represents a covalent bond;

n1 is 1;

n2 is selected from 1 and 2:

R^{y} represents hydrogen;

R³ is selected from C₁₋₆alkyl substituted with one, two, three or four substituents each independently selected from the group consisting of —NR^{xc}R^{xd}, Het¹ and Cy;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)O; wherein said heterocyclyl is optionally substituted on one nitrogen with —C(═O)—R⁸; and wherein said heterocyclyl is optionally substituted on one carbon atom with oxo;

R⁸ represents C₁₋₆alkyl; or C₁₋₆alkyl substituted with one, two or three substituents each independently selected from —OH, —O—C₁₋₄alkyl and cyano;

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to forn S(═O) or S(═O)₂; wherein said heterocyclyl is optionally substituted on one nitrogen with —C(═O)—C₁₋₄alkyl;

Cy² represents C₃₋₇cycloalkyl optionally substituted with one Het^{6a}; and the pharmaceutically acceptable salts and the solvates thereof.

The present invention relates in particular to compounds of Formula (I-x2) as defined herein, and the tautomers and the stereoisomeric forms thereof, wherein

Q represents —CHR^{y}—;

R^{1a} represents —C(═O)—NR^{xa}R^{xb};

R^{xa} and R^{xb} are C₁₋₆alkyl;

R^{1b} represents F;

R² represents methyl;

R²¹ represents hydrogen;

Y represents a covalent bond;

n1 is 1;

n2 is selected from 1 and 2;

R^{y} represents hydrogen;

R³ is selected from C₁₋₈alkyl substituted with one substituent selected from the group consisting of —NR^{xc}cR^{xd}, Het¹ and Cy²;

R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸;

R⁸ represents C₁₋₆alkyl; or C₁₋₆alkyl substituted with one, two or three substituents each independently selected from —OH, —O—C₁₋₄alkyl and cyano;

Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;

Cy² represents C₃₋₇cycloalkyl optionally substituted with one Het^{6a}; and the pharmaceutically acceptable salts and the solvates thereof.

The present invention relates in particular to compounds of Formula (I-x2) as defined herein, and the tautomers and the stereoisomeric forms thereof, wherein

Q represents —CHR^{y}—;

R^{1a} represents —C(═O)—NR^{xa}R^{xb};

R^{xa} and R^{xb} are C₁₋₈alkyl;

R^{1b} represents F;

R² represents methyl;

R²¹ represents hydrogen;

Y represents a covalent bond;

n1 is 1;

n2 is selected from 1 and 2;

R⁷ represents hydrogen;

R³ is selected from C₁₋₄alkyl substituted with one Het¹;

Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸;

R⁸ represents C₁₋₆alkyl; or C₁₋₆alkyl substituted with one, two or three substituents each independently selected from —OH, —O—C₁₋₄alkyl and cyano;

and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-y):

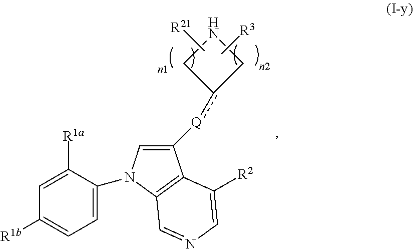

wherein the variables are as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-y1):

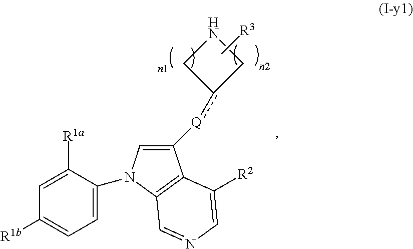

wherein the variables are as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-z):

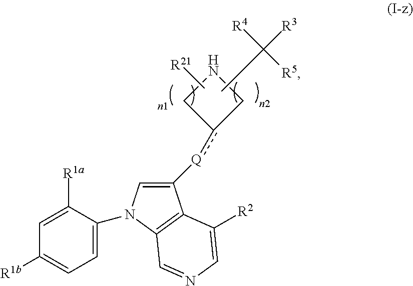

wherein the variables are as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-z1):

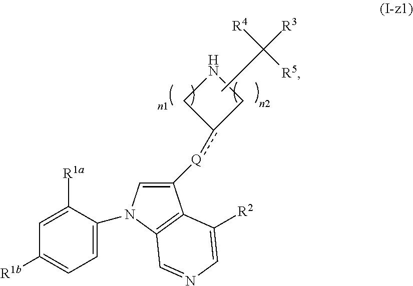

wherein the variables are as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-q):

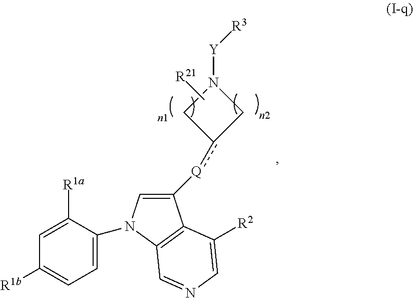

wherein the variables are as defined for the compounds of Formula (I) or any subgroup thereof as mentioned in any of the other embodiments.

In an embodiment, the present invention relates to a subgroup of Formula (I) as defined in the general reaction schemes.

In an embodiment the compound of Formula (I) is selected from the group consisting of any of the exemplified compounds, tautomers and stereoisomeric forms thereof, and the free bases, any pharmaceutically acceptable salts, and the solvates thereof.

In an embodiment the compound of Formula (I) is selected from the group consisting of compounds 4, 8, 8a, 9a, 10, 12, 18a, 18b, 20, 27a, 27d, 32a, 34a, 38b, 43, 51, 51a, 59, 60, 115, 117a, 125, 140, 157, 159, 169a, 207, 228, 258, 262 and 365b.

In an embodiment the compound of Formula (I) is selected from the group consisting of compounds 4, 8, 8a, 9a, 10, 12, 18a, 18b, 20, 27a, 27d, 32a, 34a, 38b, 43, 51, 51a, 59, 60, 115, 117a, 125, 140, 157, 159, 169a, 207, 228, 258, 262 and 365b;

tautomers and stereoisomeric forms thereof,

and the free bases, any pharmaceutically acceptable salts, and the solvates thereof.

The present invention also relates to a pharmaceutical composition comprising a therapeutically effective amount of a compound of Formula (I) and a pharmaceutically acceptable carrier or excipient, wherein the compound of Formula (I) is selected from the group consisting of any of the exemplified compounds.

The present invention also relates to a pharmaceutical composition comprising a therapeutically effective amount of a compound of Formula (I) and a pharmaceutically acceptable carrier or excipient, wherein the compound of Formula (I) is selected from the group consisting of any of the exemplified compounds, tautomers and stereoisomeric forms thereof, and the free bases, any pharmaceutically acceptable salts, and the solvates thereof.

The present invention also relates to a pharmaceutical composition comprising a therapeutically effective amount of a compound of Formula (I) and a pharmaceutically acceptable carrier or excipient, wherein the compound of Formula (I) is selected from the group consisting of compounds 4, 8, 8a, 9a, 10, 12, 18a, 18b, 20, 27a, 27d, 32a, 34a, 38b, 43, 51, 51a, 59, 60, 115, 117a, 125, 140, 157, 159, 169a, 207, 228, 258, 262 and 365b.

The present invention also relates to a pharmaceutical composition comprising a therapeutically effective amount of a compound of Formula (I) and a pharmaceutically acceptable carrier or excipient, wherein the compound of Formula (I) is selected from the group consisting of compounds 4, 8, 8a, 9a, 10, 12, 18a, 18b, 20, 27a, 27d, 32a, 34a, 38b, 43, 51, 51a, 59, 60, 115, 117a, 125, 140, 157, 159, 169a, 207, 228, 258, 262 and 365b; tautomers and stereoisomeric forms thereof, and the free bases, any pharmaceutically acceptable salts, and the solvates thereof.

In a particular embodiment, the solvate is a hydrate. In a particular embodiment, the pharmaceutically acceptable salt is a HCl salt. In a particular embodiment, the compound is a HCl salt hydrate.

In an embodiment the compound of Formula (I) is

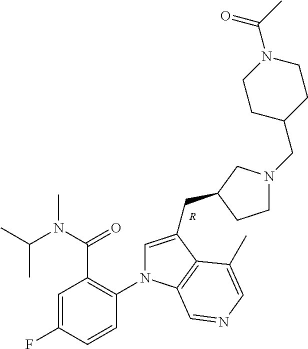

or a pharmaceutically acceptable salt or solvate thereof; in particular a HCl salt, solvate; more in particular a HCl salt, hydrate; more in particular a mono HCl salt, hydrate; even more in particular mono HCl salt, trihydrate.

All possible combinations of the above indicated embodiments are considered to be embraced within the scope of the invention.

Any aspects of the invention and embodiments described herein for the compounds of formula (I) as listed herein, also hold for the compounds of formula (A).

In an embodiment the invention relates to any of the intermediates described herein, tautomers and stereoisomeric forms thereof, and the free bases, any pharmaceutically acceptable salts, and the solvates thereof.

### Methods for the Preparation of Compounds of Formula (I)

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or prepared by standard synthetic processes commonly used by those skilled in the art of organic chemistry. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups (PG) can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

### General Synthetic Schemes

All abbreviations used in the general schemes are as defined below or as in the Table in the part Examples. Variables are as defined in the scope or as specifically defined in the general Schemes. Where compounds/intermediates in the schemes below contain a double bond, the substituents may be in the E or the Z configuration or be mixtures thereof.

In general, compounds of Formula (I-aa), can be prepared according to the following reaction Scheme 1. In Scheme 1, PG represents a suitable protecting group, such as for example tert-butyloxycarbonyl, 9-fluorenylmethoxycarbonyl, or benzyl, and LG is a leaving group such as for example chloro, bromo, iodo or tosylate or mesylate or triflate; all other variables are defined according to the scope of the present invention.

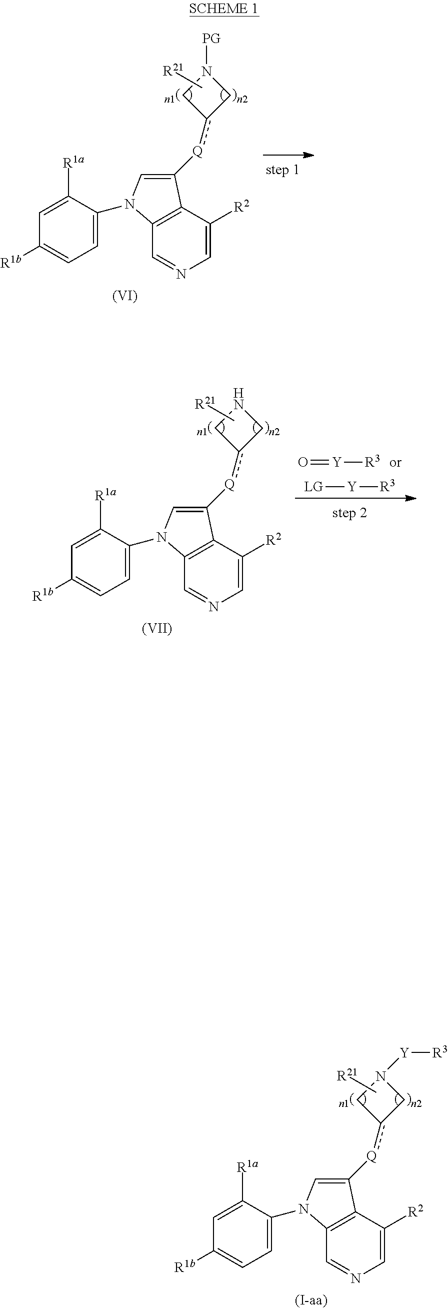

In Scheme 1, the following reaction conditions apply:

Step 1: when PG=Boc, at a suitable temperature in a range between 0° C. and 40° C., such as room temperature, in the presence of a suitable acid, for example a protic acid such as trifluoroacetic acid (TFA) or hydrochloric acid, in a suitable solvent such as dichloromethane (DCM) or 1,4-dioxane;

Alternatively, when PG=9-fluorenylmethoxycarbonyl, at a suitable temperature in a range between 0° C. and 40° C., such as room temperature, in the presence of a suitable base such as piperidine, in a suitable solvent such as dichloromethane (DCM);

Alternatively, when PG=benzyl, at a suitable temperature such as room temperature, in the presence of a suitable heterogenous catalyst such as palladium on charcoal (Pd/C), in a common solvent such as methanol, ethanol, TIF or the like under hydrogen pressure such as for example from 1 to 3 bar, optionally in the presence of a base such as triethylamine;

### Step 2:

In the case of a reductive amination reaction employing an aldehyde or a ketone: at a suitable temperature in a range between room temperature and 70° C., in the presence of a suitable reducing agent such as for example sodium triacetoxyborohydride or sodium cyanoborohydride, in a suitable solvent such as for example methanol, dichloromethane or 1,2-dichloroethane, optionally in the presence of zinc chloride or sodium acetate or acetic acid;

In the case of an alkylation reaction employing LG-Y—R³: at a suitable temperature such as for example room temperature, in the presence of a suitable deprotonating agent such as for example sodium hydride or potassium carbonate, or an amine base such as triethylamine in a suitable aprotic solvent such as for example dimethylformamide or dimethylsulfoxide or acetonitrile.

In general, compounds of Formula (I) wherein Q is limited to —O—, —NR^{q}—, can be prepared via intermediates of Formula (VIc). Intermediates of Formula (VIc) can be prepared according to the following reaction Scheme 2. In Scheme 2, PG represents a suitable protecting group, such as for example tert-butyloxycarbonyl; all other variables are defined according to the scope of the present invention.

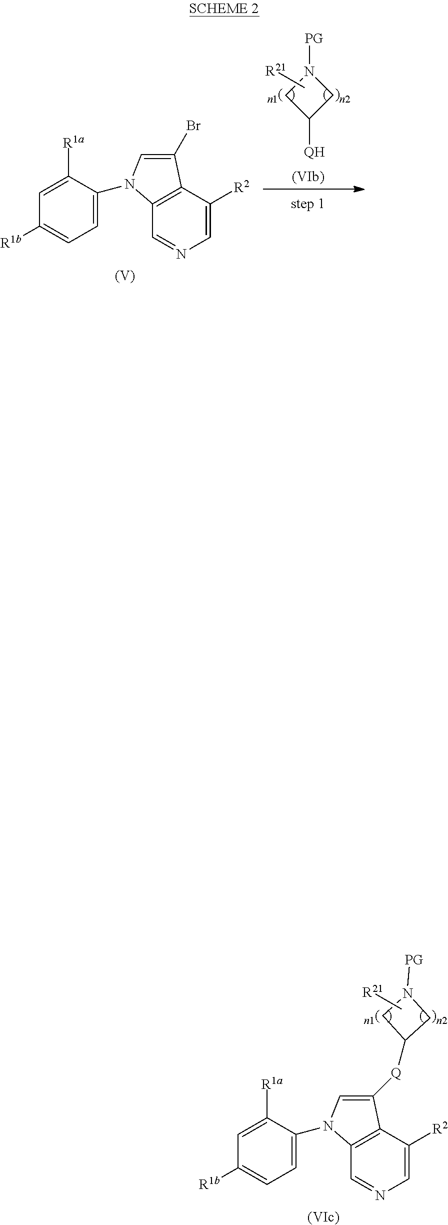

In Scheme 2, the following reaction conditions apply:

Step 1: at a suitable temperature in a range between 100° C. and 140° C., in the presence of a suitable base such as for example potassium tert-butoxide or potassium phosphate, in the presence of a suitable catalyst such as palladium acetate (Pd(OAc)₂) or tris(dibenzylideneacetone)dipalladium(0) (Pd₂dba₃) or [1,1′-bis(diphenylphosphino)ferrocene]dichloropalladium(II) (Pd(dppf)Cl₂), in the presence of a suitable ligand such as 9,9-dimethyl-4,5-bis(diphenylphosphino)xanthene (Xantphos), in a suitable solvent such as for example dioxane or dimethylformamide.

In general, intermediates of Formula (V) can be prepared according to the following reaction Scheme 2B. In Scheme 2B, W¹ represents fluoro, chloro, bromo or iodo, BPin represents 4,4,5,5-tetramethyl-1,3,2-dioxaborolane, and all other variables are defined according to the scope of the present invention.

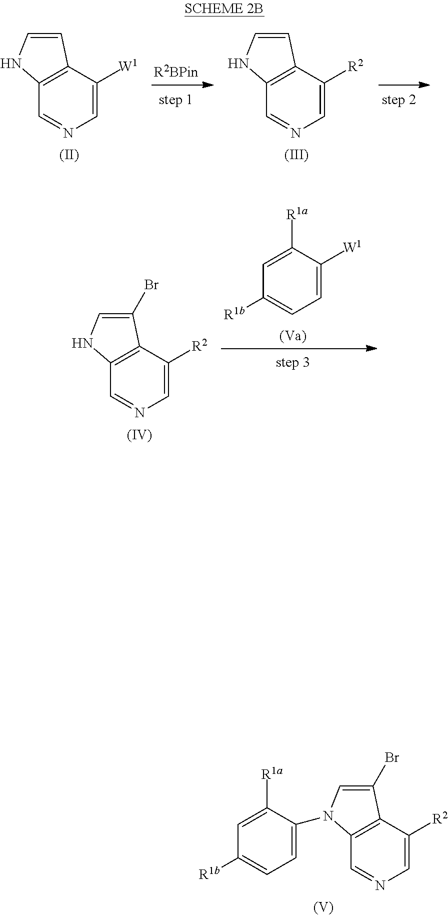

In Scheme 2B, the following reaction conditions apply:

Step 1: at a suitable temperature in a range between room temperature and 100° C., in the presence of a suitable base such as for example potassium carbonate, in the presence of a suitable catalyst such as [1,1′-Bis(diphenylphosphino)ferrocene]dichloropalladium(II) (Pd(dppf)Cl₂) in a suitable solvent such as for example dioxane or dimethylformamide and water; Alternatively, when R²=Me, a boron containing reagent such as trimethyl boroxine can be used in the presence of a suitable catalyst such as (Pd(dppf)Cl₂) in a suitable solvent such as for example dioxane or dimethylformamide and water in the presence of an inorganic base such as potassium carbonate at a reaction temperature between 80° C. and 120° C.;

Additional step to achieve the double bond reduction to obtain R² is C₃₋₆cycloalkyl, C₁₋₄alkyl, or C₁₋₄alkyl substituted with one, two or three halo substituents: at a suitable temperature such as room temperature, in the presence of a suitable catalyst such as palladium on charcoal (Pd/C), in a suitable solvent such as methanol, under H₂ pressure such as for example from 1 to 3 bar, optionally in the presence of a base such as triethylamine;

Step 2: at a suitable temperature such as for example between 0° C. and room temperature, in the presence of a suitable bronination reagent such as for example N-Bromosuccinimide or CuBr₂, in a suitable solvent such as for example dimethylformamide or acetonitrile;

Step 3: at a suitable temperature such as for example 80° C. and 130° C., in the presence of a suitable catalyst such as copper (Cu), in the presence of a base such as potassium carbonate, in a suitable solvent such as dimethylformamide; Alternatively a copper (I) source may be used, such as CuI in the presence of a suitable diamine ligand, such as trans-N,N′-dimethylcyclohexane-1,2-diamine in the presence of an inorganic base, such as potassium carbonate in an aprotic solvent such as dimethylformamide at a temperature between 80° C. and 150° C. In certain cases said conversion may also be effected by a nucleophilic aromatic substitution using an inorganic base such as potassium tert-butoxide or sodium hydride or the like, in an aprotic solvent such as dimethylformamide at a temperature between 0° C. and 80° C.;

Someone skilled in the art will appreciate that the steps 2 and 3 in Scheme 2B may also be reversed, i.e. first the cross coupling of intermediate (III) with the reagent (Va), followed by bromination of the aza indole moiety to provide the intermediate (V).

In general, intermediates of Formula (VIIa), can be prepared via intermediates of Formula (VIe). Intermediates of Formula (VIe) can also be prepared according to the following reaction Scheme 3. In Scheme 3, PG represents a suitable protecting group, such as for example tert-butyloxycarbonyl; all other variables are defined according to the scope of the present invention.

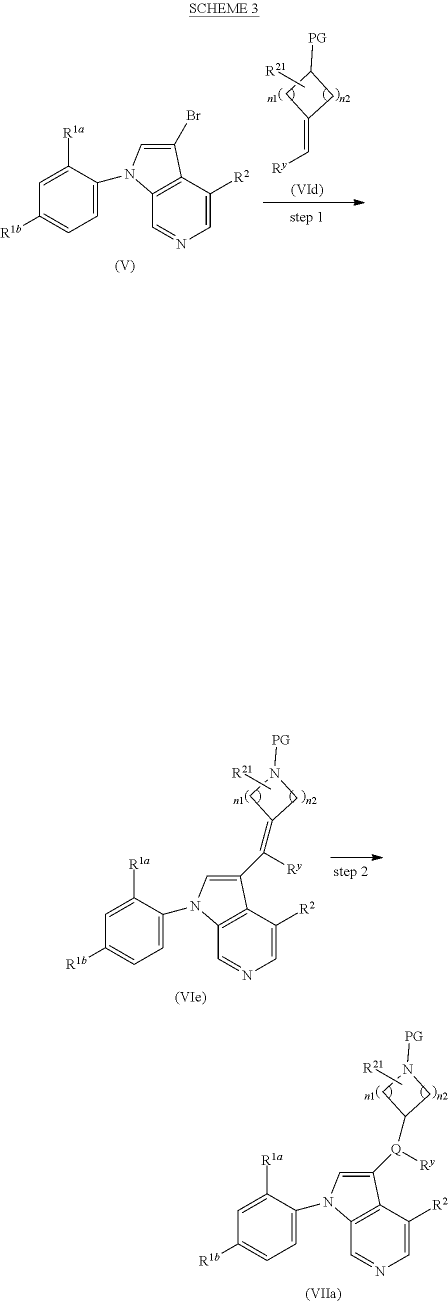

In Scheme 3, the following reaction conditions apply:

Step 1: at a suitable temperature in a range between 70° C. and 100° C., in the presence of a suitable base such as for example potassium phosphate, in presence of a suitable catalyst such as palladium acetate (Pd(OAc)₂), optionally in the presence of a suitable phosphine ligand such as 2-dicyclohexylphosphino-2′-(N,N-dimethylamino)biphenyl (Davephos), in a suitable solvent such as for example dioxane or dimethylformamide;

Step 2: at a suitable temperature such as room temperature, in the presence of a suitable heterogenous catalyst such as palladium on charcoal (Pd/C), in a common solvent such as methanol, ethanol, THF or the like under hydrogen pressure such as for example from 1 to 3 bar, optionally in the presence of a base such as triethylamine;

In general, intermediates of Formula (IXb) can be prepared according to the following reaction Scheme 4. In Scheme 4 PG represents a suitable protecting group, such as for example tert-butyloxycarbonyl; all other variables are defined according to the scope of the present invention.

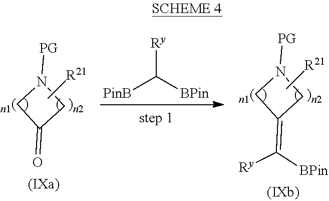

Step 1: at a suitable temperature such as for example −78° C., in the presence of a suitable deprotonating agent such as for example n-Butyllithium, in presence of a suitable reagent such as 2,2,6,6-Tetramethylpiperidine (HTMP), in a suitable solvent such as tetrahydrofuran;

In general, compounds of Formula (I-a), can be prepared according to the following reaction Scheme 5. In Scheme 5, PG represents a suitable protecting group, such as for example tert-butyloxycarbonyl and LG is a leaving group such as for example chloro, bromo, iodo or tosylate or mesylate or triflate; all other variables are defined according to the scope of the present invention.

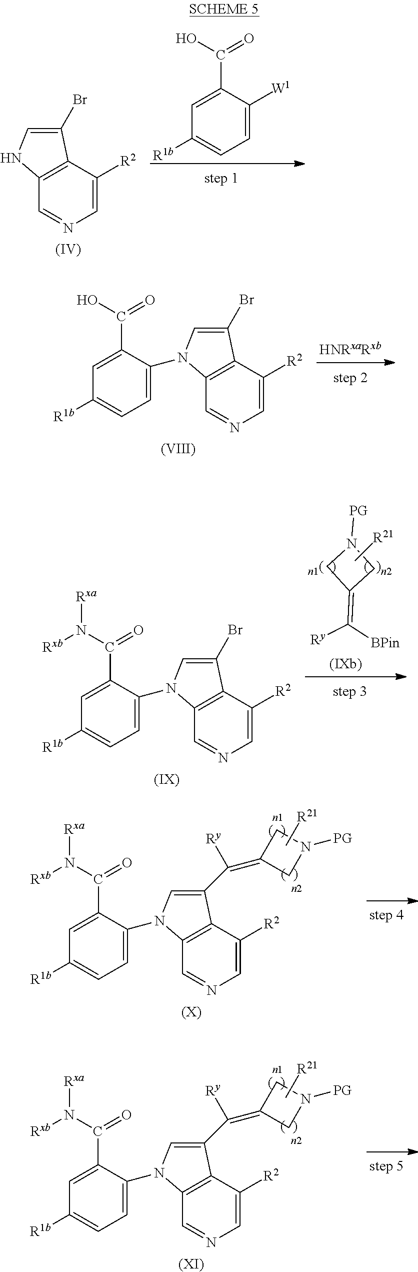

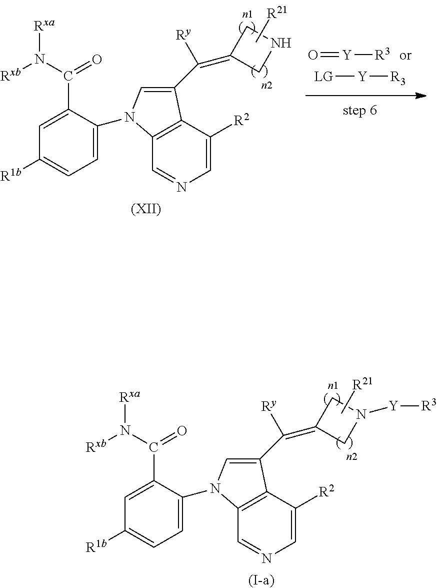

In Scheme 5, the following reaction conditions apply:

Step 1: at a suitable temperature such as for example 100° C., in the presence of a suitable catalyst such as copper (Cu), in the presence of a base such as potassium carbonate, in a suitable solvent such as dimethylformamide; Alternatively a copper (I) source may be used, such as CuI in the presence of a suitable diamine ligand, such as trans-N,N′-dimethylcyclohexane-1,2-diamine, in the presence of an inorganic base, such as potassium carbonate in an aprotic solvent such as dimethylformamide at a temperature between 80° C. and 150° C.;

Step 2: at a suitable temperature such as for example room temperature, in the presence of a suitable condensation reagent such as 2-(7-Azabenzotriazol-1-yl)-N,N,N′,N′-tetramethyluronium hexafluorophosphate (HATU), in the presence of a base such as N,N-Diisopropylethylamine (DIPEA), in a suitable solvent such as dimethylformamide; Alternatively the acid chloride may be prepared by reacting intermediate VIII with thionyl chloride optionally in a halogenated solvent such as dichloromethane at a temperature in a range between 0° C. and room temperature. The intermediate acid chloride may then be reacted with the amine HNR^{xa}R^{xb} optionally in an aprotic solvent such as dimethylformamide and optionally in the presence of a tertiary amine such as N,N-diisopropylethylamine;

Step 3: at a suitable temperature in a range between 60° C. and 120° C., such as for example 100° C., in the presence of a suitable base such as for example potassium carbonate, in presence of a suitable catalyst such as [1,1′-Bis(diphenylphosphino)ferrocene]dichloropalladium(II) (Pd(dppf)Cl₂) in a suitable solvent such as for example dioxane or dimethylformamide and water;

Step 4: at a suitable temperature such as room temperature, in the presence of a suitable heterogenous catalyst such as palladium on charcoal (Pd/C), in a common solvent such as methanol, ethanol, THF or the like under hydrogen pressure such as for example from 1 to 3 bar, optionally in the presence of a base such as triethylamine;

Step 5: at a suitable temperature in a range between 0° C. and 40° C., such as room temperature, in the presence of a suitable acid, for example a protic acid such as trifluoroacetic acid (TFA) or hydrochloric acid, in a suitable solvent such as dichloromethane (DCM) or 1,4-dioxane; Step 6:

In the case of a reductive amination reaction employing an aldehyde or a ketone: at a suitable temperature in a range between room temperature and 70° C., in the presence of a suitable reducing agent such as for example sodium triacetoxyborohydride or sodium cyanoborohydride, in a suitable solvent such as for example methanol, dichloromethane or 1,2-dichloethane, optionally in the presence of zinc chloride or sodium acetate or acetic acid;

In the case of an alkylation reaction employing LG-Y—R³: at a suitable temperature such as for example room temperature, in the presence of a suitable deprotonating agent such as for example sodium hydride or potassium carbonate, or an amine base such as triethylamine in a suitable aprotic solvent such as for example dimethylformamide or dimethylsulfoxide or acetonitrile.

In general, compounds of Formula (I-b) wherein R^{1a} is limited to —S(═O)₂—R¹⁸,

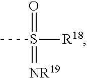

and Q represents —CHR^{y}—, can be prepared according to the following reaction Scheme 6. In Scheme 6, PG represents a suitable protecting group, such as for example tert-butyloxycarbonyl, 9-fluorenylmethoxycarbonyl, or benzyl; LG is a leaving group such as for example chloro, bromo, iodo or tosylate or mesylate; LGI is a leaving group such as for example fluoro, chloro, bromo, iodo or tosylate or mesylate; all other variables are defined according to the scope of the present invention.

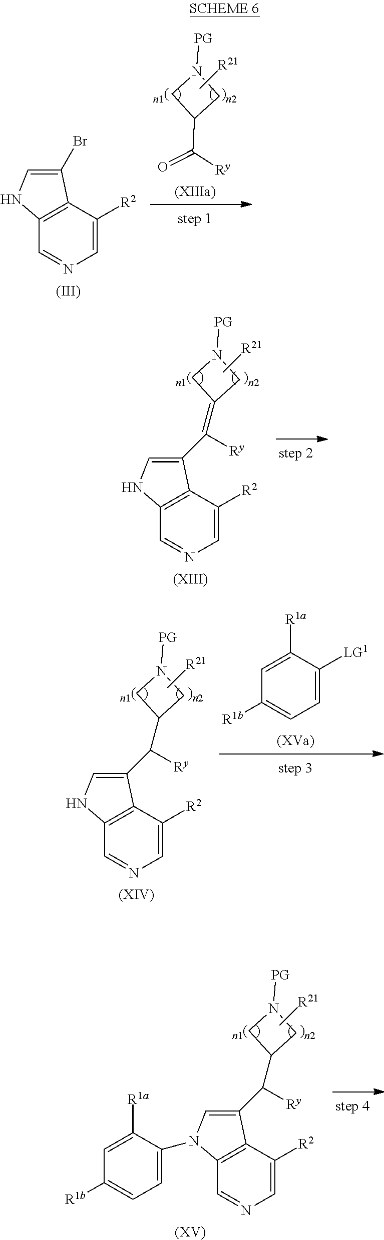

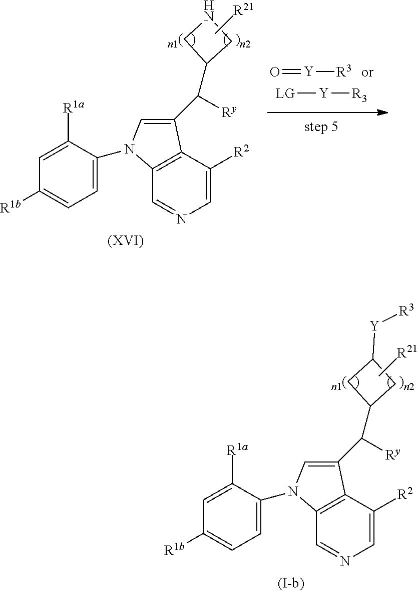

In Scheme 6, the following reaction conditions apply:

Step 1: at a suitable temperature in a range between 50° C. and 90° C., in the presence of a suitable base such as for example potassium hydroxide or sodium hydroxide, in a suitable solvent, preferably a protic solvent, such as methanol, ethanol or isopropanol.

Step 2: at a suitable temperature such as room temperature, in the presence of a suitable heterogenous catalyst such as palladium on charcoal (Pd/C), in a common solvent such as methanol, ethanol, THF or the like under hydrogen pressure such as for example from 1 to 3 bar, optionally in the presence of a base such as triethylamine;

Step 3: at a suitable temperature in a range between 50° C. and 100° C., in the presence of a suitable inorganic base such as for example potassium carbonate or potassium tert-butoxide, in a suitable aprotic solvent such as for example dioxane, dimethylformamide or acetonitrile or dimethylsulfoxide;

Step 4: when PG=Boc, at a suitable temperature in a range between 0° C. and 40° C., such as room temperature, in the presence of a suitable acid, for example a protic acid such as trifluoroacetic acid or hydrochloric acid, in a suitable solvent such as dichloromethane or 1,4-dioxane;

Alternatively, when PG=9-Fluorenylmethoxycarbonyl, at a suitable temperature in a range between 0° C. and 40° C., such as room temperature, in the presence of a suitable base such as piperidine, in a suitable solvent such as dichloromethane (DCM);

Alternatively, when PG=benzyl, at a suitable temperature such as room temperature, in the presence of a suitable heterogenous catalyst such as palladium on charcoal (Pd/C), in a common solvent such as methanol, ethanol, THF or the like under hydrogen pressure such as for example from 1 to 3 bar, optionally in the presence of a base such as triethylamine;

Step 5: In the case of a reductive amination reaction employing an aldehyde or a ketone: at a suitable temperature in a range between room temperature and 70° C., in the presence of a suitable reducing agent such as for example sodium triacetoxyborohydride or sodium cyanoborohydride, in a suitable solvent such as for example methanol, dichloromethane or 1,2-dichloroethane, optionally in the presence of zinc chloride or sodium acetate or acetic acid;

In the case of an alkylation reaction employing LG-Y—R³: at a suitable temperature such as for example room temperature, in the presence of a suitable deprotonating agent such as for example sodium hydride or potassium carbonate, or an amine base such as triethylamine in a suitable aprotic solvent such as for example dimethylformamide or dimethylsulfoxide or acetonitrile;

In general, compounds of Formula (I-c), can be prepared according to the following reaction Scheme 7. In Scheme 7, PG represents a suitable protecting group, such as for example tert-butyloxycarbonyl, 9-fluorenylmethoxycarbonyl, or benzyl, all other variables are defined according to the scope of the present invention.

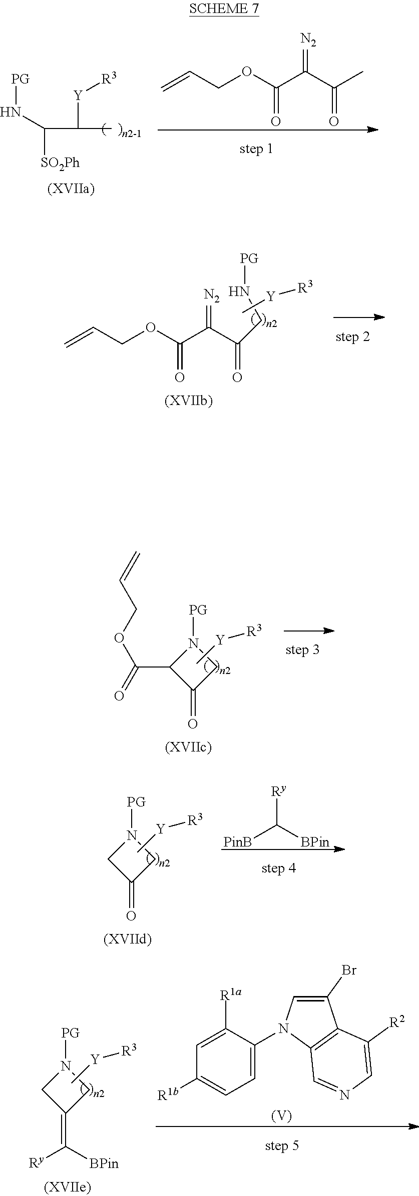

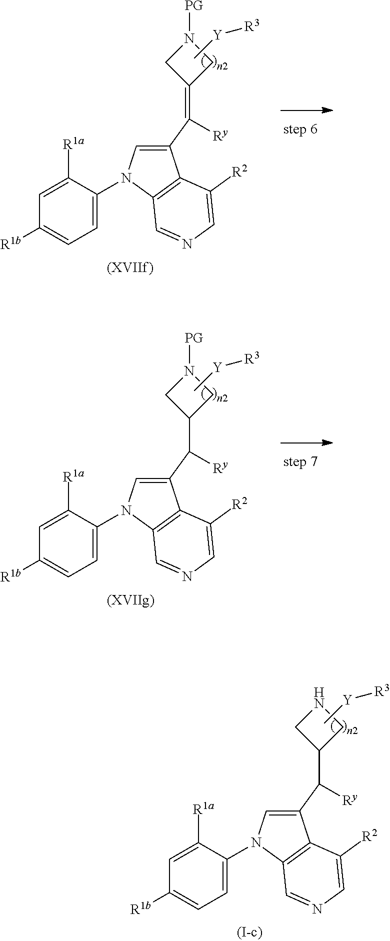

In Scheme 7, the following reaction conditions apply:

Step 1: at a suitable temperature such as for example −78° C., in the presence of a suitable deprotonating agent such as for example lithium bis(trimethylsilyl)amide (LiHMDS) and sodium hydride, in a suitable solvent such as for example tetrahydrofuran;

Step 2: at a suitable temperature in a range between room temperature and 100° C., in the presence of a suitable catalyst such as for example rhodium acetate dimer (Rh₂(OAc)₄), in a suitable solvent such as for example dichloromethane;

Step 3: at a suitable temperature in a range between room temperature and 100° C., in the presence of a suitable catalyst such as for example tetrakis(triphenylphosphine)palladium (Pd(PPh₃)₄), in the presence of a suitable base such as for example morpholine, in a suitable solvent such as for example tetrahydrofuran;

Step 4: at a suitable temperature such as for example −78° C., in the presence of a suitable deprotonating agent such as for example n-Butyllithium, in presence of a suitable reagent such as 2,2,6,6-Tetramethylpiperidine (HTMP), in a suitable solvent such as tetrahydrofuran;

Step 5: at a suitable temperature such as for example 100° C., in the presence of a suitable base such as for example potassium carbonate, in presence of a suitable catalyst such as [1,1′-Bis(diphenylphosphino)ferrocene]dichloropalladium(II) (Pd(dppf)Cl₂) in a suitable solvent such as for example dioxane or dimethylformamide and water;

Step 6: at a suitable temperature such as room temperature, in the presence of a suitable heterogenous catalyst such as palladium on charcoal (Pd/C), in a common solvent such as methanol, ethanol, THF or the like under hydrogen pressure such as for example from 1 to 3 bar, optionally in the presence of a base such as triethylamine;

Step 7: when PG=Boc, at a suitable temperature in a range between 0° C. and 40° C., such as room temperature, in the presence of a suitable acid, for example a protic acid such as trifluoroacetic acid or hydrochloric acid, in a suitable solvent such as dichloromethane or 1,4-dioxane;

Alternatively, when PG=9-fluorenylmethoxycarbonyl, at a suitable temperature in a range between 0° C. and 40° C., such as room temperature, in the presence of a suitable base such as piperidine, in a suitable solvent such as dichloromethane (DCM);

Alternatively, when PG=benzyl, at a suitable temperature such as room temperature, in the presence of a suitable heterogenous catalyst such as palladium on charcoal (Pd/C), in a common solvent such as methanol, ethanol, THF or the like under hydrogen pressure such as for example from 1 to 3 bar, optionally in the presence of a base such as triethylamine.

An example of steps 1 and 2 in Scheme 7 is the preparation of a 5-membered intermediate (XVIIcc), as shown in Scheme 7a which can be prepared according to the general procedures outlined in steps 1 and 2 in Scheme 7.

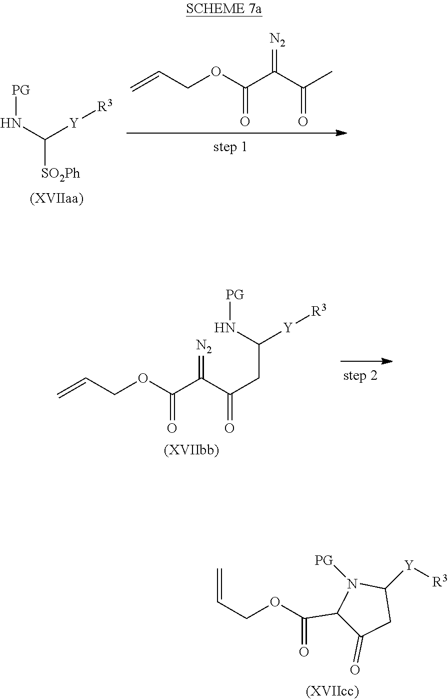

In general, intermediates of Formula (XVIIId), can be prepared according to the following reaction Scheme 8. In Scheme 8, W₂ represents chloro, bromo or iodo, all other variables are defined according to the scope of the present invention. A skilled person will realize that cyclobutyl in Scheme 8 can be C₃₋₅cycloalkyl in general, and that an intermediate of Formula (XVIIId) can be further functionalized into a compound of Formula (I) by analogous reaction protocols as described in the general schemes herein, combined with standard synthetic processes commonly used by those skilled in the art of organic chemistry.

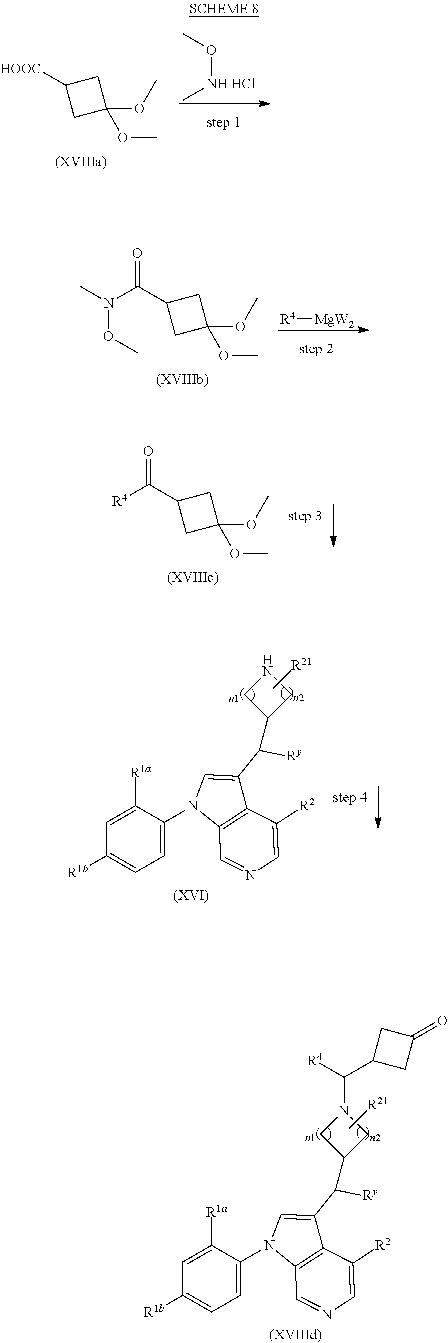

In Scheme 8, the following reaction conditions apply:

Step 1: at a suitable temperature such as for example 0° C., in the presence of a suitable condensation reagent such as propylphosphonic anhydride (T₃P), in the presence of a base such as N,N-Diisopropylethylamine (DIPEA), in a suitable solvent such as dimethylformamide or dichloromethane;

Step 2: at a suitable temperature such as for example 0° C., in a suitable solvent such as tetrahydrofuran;

Step 3: at a suitable temperature in a range between room temperature and 70° C., in the presence of a suitable reducing agent such as for example sodium triacetoxyborohydride or sodium cyanoborohydride, in a suitable solvent such as for example methanol, dichloromethane or 1,2-dichloroethane, optionally in the presence of zinc chloride or sodium acetate or acetic acid;

Step 4: at a suitable temperature in a range between 0° C. and 40° C., such as room temperature, in the presence of a suitable acid such as hydrochloric acid (HCl, 1N), in a suitable solvent such as acetonitrile.

In general, intermediates as described in Scheme 9, wherein Q represents —CHR^{y}—, can be prepared according to the following reaction Scheme. In Scheme 9, PG represents a suitable protecting group, such as for example tert-butyloxycarbonyl, all other variables are defined according to the scope of the present invention.

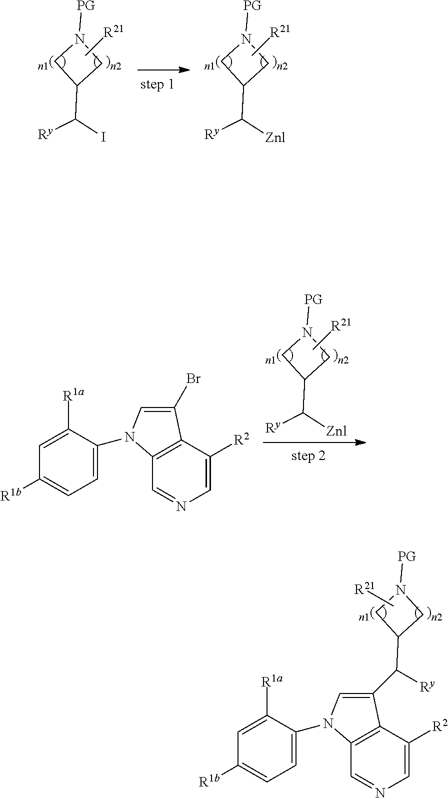

In Scheme 9, the following reaction conditions apply:

Step 1: at a suitable temperature, in a range between room temperature and 70° C., such as 60° C., in the presence of zinc, in the presence of suitable activating agents such as trimethylsilylchloride or 1-bromo, 2-chloroethane, in a suitable solvent such as tetrahydrofuran. Optionally, the procedure can also be performed with the use of a flow-apparatus;

Step 2: at a suitable temperature, in a range between room temperature and 70° C., such as 50° C., in the presence of a suitable catalyst such as 4^{th} generation RuPhos Pd precatalyst (RuPhos Pd G4), in a suitable solvent such as tetrahydrofuran.

In general, intermediates as described in Scheme 10, wherein Q represents —CHR^{y}—, can be prepared according to the following reaction Scheme. In Scheme 10, PG represents a suitable protecting group, such as for example tert-butyloxycarbonyl, all other variables are defined according to the scope of the present invention. BPin represents 4,4,5,5-tetramethyl-1,3,2-dioxaborolane. W¹ and W³ represent fluoro, chloro, bromo or iodo.

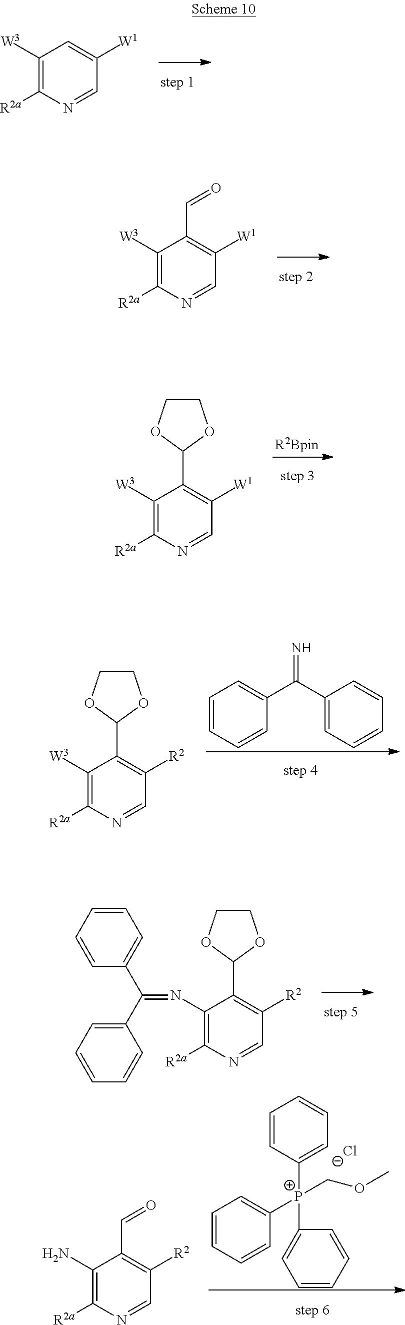

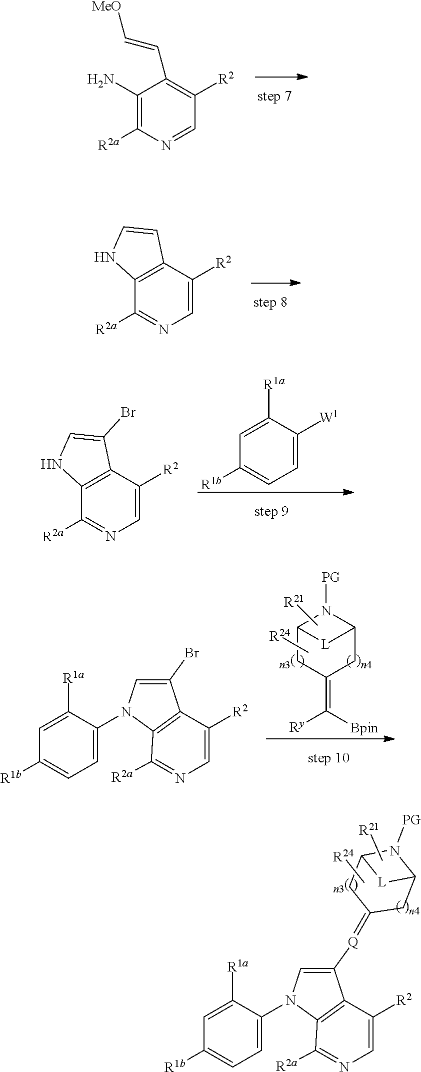

Step 1: at a suitable temperature, such as −78° C., in the presence of a suitable deprotonating agent such as n-Butyllithium, in a suitable solvent such as tetrahydrofuran, in the presence of suitable electrophile, such as DMF;

Step 2: at a suitable temperature in a range between 80° C. and 120° C., in the presence of a diol protection reagent such as for example glycol, in the presence of a Bronsted acid such as for example para-toluenesulfonic acid in a suitable solvent such as for example toluene;

Step 3: at a suitable temperature in a range between room temperature and 100° C., in the presence of a suitable base such as for example potassium carbonate, in the presence of a suitable catalyst such as [1,1′-Bis(diphenylphosphino)ferrocene]dichloropalladium(II) (Pd(dppf)Cl₂) in a suitable solvent such as for example dioxane or dimethylformamide and water; Alternatively, when R²=Me, a boron containing reagent such as trimethyl boroxine can be used in the presence of a suitable catalyst such as (Pd(dppf)Cl₂) in a suitable solvent such as for example dioxane or dimethylformamide and water in the presence of an inorganic base such as potassium carbonate at a reaction temperature between 80° C. and 120° C.; Step 4: in the presence of a suitable base, such as for example sodium t-butoxide, in the presence of a suitable palladium source such as palladium(II)acetate (Pd(OAc)₂), in the presence of a suitable ligand, such as 1,1′-(9,9-Dimethyl-9H-xanthene-4,5-diyl)bis[1,1-diphenylphosphine], Xantphos, in the presence of a suitable solvent, such as 1,4-dioxane, at a suitable temperature range between 50° C. and 120° C.;

Step 5: in the presence of a suitable Bronsted acid, such as hydrochloric acid, in the presence of a suitable solvent such as 1,4-dioxane or tetrahydrofuran, and water, at a suitable temperature range such a room temperature and 60° C.;

Step 6: in the presence of a suitable deprotonating agent, such as n-butyllithium, in a suitable solvent such as tetrahydrofuran, at a suitable temperature range such as −78° C. and room temperature.

Step 7: in the presence of a suitable Bronsted acid, such as hydrochloric acid, in the presence of a suitable solvent such as 1,4-dioxane or tetrahydrofuran, and water, at a suitable temperature range such a room temperature and 100° C.;

Step 8: at a suitable temperature such as for example between 0° C. and room temperature, in the presence of a suitable bromination reagent such as for example N-bromosuccinimide or CuBr₂, in a suitable solvent such as for example dimethylformamide or acetonitrile;

Step 9: at a suitable temperature such as for example 80° C. and 130° C., in the presence of a suitable catalyst such as copper (Cu), in the presence of a base such as potassium carbonate, in a suitable solvent such as dimethylformamide. Alternatively a copper (I) source may be used, such as CuI in the presence of a suitable diamine ligand, such as trans-N,N′-dimethylcyclohexane-1,2-diamine in the presence of an inorganic base, such as potassium carbonate in an aprotic solvent such as dimethylformamide at a temperature between 80° C. and 150° C. In certain cases said conversion may also be effected by a nucleophilic aromatic substitution using an inorganic base such as potassium tert-butoxide or sodium hydride or the like, in an aprotic solvent such as dimethylformamide at a temperature between 0° C. and 80° C.;

Step 10: at a suitable temperature such as for example 100° C., in the presence of a suitable base such as for example potassium carbonate, in presence of a suitable catalyst such as [1,1′-Bis(diphenylphosphino)ferrocene]dichloropalladium(II) (Pd(dppf)Cl₂) in a suitable solvent such as for example dioxane or dimethylformamide and water.

As can be appreciated by a person skilled in the art, the intermediate obtained in scheme 10, can be further elaborated to obtain compounds of Formula (A) by means of using the procedures outlined in the general schemes mentioned above, in particular in scheme 1 and scheme 3.

In general, intermediates as described in Scheme 11, can be prepared according to the following reaction Scheme. In Scheme 11, PG represents a suitable protecting group, such as for example tert-butyloxycarbonyl, all other variables are defined according to the scope of the present invention. BPin represents 4,4,5,5-tetramethyl-1,3,2-dioxaborolane.

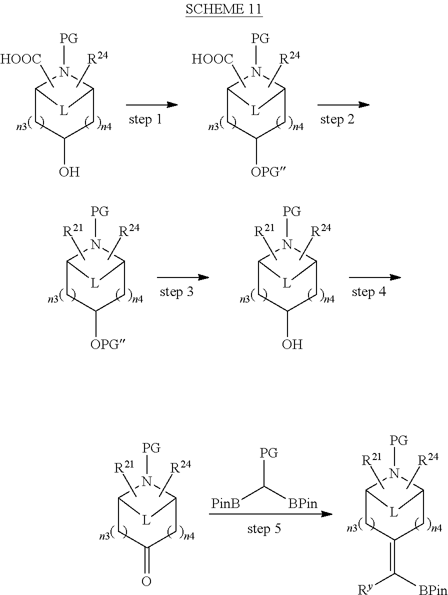

Step 1: when PG″=a silyl containing protecting group, such as tert-butyldimethylsilyl, at a suitable temperature in a range between room temperature and 80° C., such as room temperature, in the presence of a base, such as imidazole, in the presence of a suitable reagent, such as tert-butyldimethylsilylchloride, in a suitable solvent, such as DMF. When, PG″ is a different protecting group as defined herein, general protection conditions may be used, known to those skilled in the art.

Step 2: at a suitable temperature, between room temperature and 60° C., such as room temperature, for example in the presence of a suitable alkyl halide, in the presence of a suitable base, such as K₂CO₃, in the presence of a suitable photocatalyst, such as [4,4′-Bis(1,1-dimethylethyl)-2,2′-bipyridine-N¹,N^{1′}]bis[3,5-difluoro-2-[5-(trifluoromethyl)-2-pyridinyl-N]phenyl-C]Iridium(III) hexafluorophosphate, [Ir{dF(CF₃)ppy}₂(dtbpy)]PF₆, in the presence of suitable nickel salt, such as NiCl₂-glyme, in the presence of a suitable ligand, such as 4-4′-dimethoxy-2-2′-bipyridine, in a suitable solvent, such as acetonitrile and in the presence of water as an additive, employing blue LED irradiation (Johnston, C., Smith, R., Allmendinger, S. et al. Metallaphotoredox-catalysed sp³-sp³ cross-coupling of carboxylic acids with alkyl halides. Nature 536, 322-325 (2016)).

Step 3: at a suitable temperature, such as room temperature, in the presence of a suitable fluoride source, such a tetrabutylammonium fluoride, in a suitable solvent, such as tetrahydrofuran. When, PG is a different protecting group as defined herein, general protection conditions may be used, known to those skilled in the art.

Step 4: at a suitable temperature, such as between −78° C. and 40° C., in the presence of Dess-Martin periodinane, in a suitable solvent such as dichloromethane. Other oxidation methods, known to those skilled in the art may also be employed.

Step 5: at a suitable temperature such as for example −78° C., in the presence of a suitable deprotonating agent such as for example n-Butyllithium, in presence of a suitable reagent such as 2,2,6,6-Tetramethylpiperidine (HTMP), in a suitable solvent such as tetrahydrofuran.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl (CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, N.J., 2007.

### Pharmacology

It has been found that the compounds of the present invention block the interaction of menin with MLL proteins and oncogenic MLL fusion proteins per se, or can undergo metabolism to a (more) active form in vivo (prodrugs). Therefore the compounds according to the present invention and the pharmaceutical compositions comprising such compounds may be useful for the treatment or prevention, in particular treatment, of diseases such as cancer, including but not limited to leukemia, myelodysplastic syndrome (MDS), and myeloproliferative neoplasms (MPN); and diabetes.

In particular, the compounds according to the present invention and the pharmaceutical compositions thereof may be useful in the treatment or prevention of cancer. According to one embodiment, cancers that may benefit from a treatment with menin/MLL inhibitors of the invention comprise leukemias, lymphomas, myelomas or solid tumor cancers (e.g. prostate cancer, lung cancer, breast cancer, pancreatic cancer, colon cancer, liver cancer, melanoma and glioblastoma, etc.). In some embodiments, the leukemias include acute leukemias, chronic leukemias, myeloid leukemias, myelogeneous leukemias, lymphoblastic leukemias, lymphocytic leukemias, Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, leukemias exhibiting HOX/MEIS1 gene expression signatures etc.

In particular, the compounds according to the present invention and the pharmaceutical compositions thereof may be useful in the treatment or prevention of myelodysplastic syndrome (MDS) or myeloproliferative neoplasms (MPN).

In particular, compounds according to the present invention and the pharmaceutical compositions thereof may be useful in the treatment or prevention of leukemias, in particular nucleophosmin (NPM1)-mutated leukemias, e.g. NPM1c.

In particular, compounds according to the present invention and the pharmaceutical compositions thereof may be useful in the treatment or prevention of AML, in particular nucleophosmin (NPMT)-mutated AML (i.e., NPM1^{mut} AML), more in particular abstract NPM1-mutated AML.

In particular, compounds according to the present invention and the pharmaceutical compositions thereof may be useful in the treatment or prevention of MLL-rearranged leukemias, in particular MLL-rearranged AML or ALL.

In particular, compounds according to the present invention and the pharmaceutical compositions thereof may be useful in the treatment or prevention of leukemias with MLL gene alterations, in particular AML or ALL with MLL gene alterations.

In particular, compounds according to the present invention and the pharmaceutical compositions thereof may be suitable for Q.D. dosing (once daily).

In particular, compounds according to the present invention and the pharmaceutical compositions thereof may be useful in the treatment or prevention of hematological cancer in a subject exhibiting NPM1 gene mutations and/or mixed lineage leukemia gene (MLL; MLL1; KMT2A) alterations, mixed lineage leukemia (MLL), MLL-related leukemia, MLL-associated leukemia, MLL-positive leukemia, MLL-induced leukemia, rearranged mixed lineage leukemia, leukemia associated with a MLL, rearrangement/alteration or a rearrangement/alteration of the MLL gene, acute leukemia, chronic leukemia, myelodysplastic syndrome (MDS), myeloproliferative neoplasms (MPN), insulin resistance, pre-diabetes, diabetes, or risk of diabetes, hyperglycemia, chromosomal rearrangement on chromosome 11q23, type-1 diabetes, type-2 diabetes; promoting proliferation of a pancreatic cell, where pancreatic cell is an islet cell, beta cell, the beta cell proliferation is evidenced by an increase in beta cell production or insulin production; and for inhibiting a menin-MLL interaction, where the MLL fusion protein target gene is HOX or MEIS1 in human.

Hence, the invention relates to compounds of Formula (I), the tautomers and the stereoisomeric forms thereof, and the pharmaceutically acceptable salts, and the solvates thereof, for use as a medicament.

The invention also relates to the use of a compound of Formula (I), a tautomer or a stereoisomeric form thereof, or a pharmaceutically acceptable salt, or a solvate thereof, or a pharmaceutical composition according to the invention, for the manufacture of a medicament.

The present invention also relates to a compound of Formula (I), a tautomer or a stereoisomeric form thereof, or a pharmaceutically acceptable salt, or a solvate thereof, or a pharmaceutical composition according to the invention, for use in the treatment, prevention, amelioration, control or reduction of the risk of disorders associated with the interaction of menin with MLL proteins and oncogenic MLL fusion proteins in a mammal, including a human, the treatment or prevention of which is affected or facilitated by blocking the interaction of menin with MLL proteins and oncogenic MLL fusion proteins.

Also, the present invention relates to the use of a compound of Formula (I), a tautomer or a stereoisomeric form thereof, or a pharmaceutically acceptable salt, or a solvate thereof, or a pharmaceutical composition according to the invention, for the manufacture of a medicament for treating, preventing, ameliorating, controlling or reducing the risk of disorders associated with the interaction of menin with MLL proteins and oncogenic MLL fusion proteins in a mammal, including a human, the treatment or prevention of which is affected or facilitated by blocking the interaction of menin with MLL proteins and oncogenic MLL fusion proteins.

The invention also relates to a compound of Formula (I), a tautomer or a stereoisomeric form thereof, or a pharmaceutically acceptable salt, or a solvate thereof, for use in the treatment or prevention of any one of the diseases mentioned hereinbefore.

The invention also relates to a compound of Formula (I), a tautomer or a stereoisomeric form thereof, or a pharmaceutically acceptable salt, or a solvate thereof, for use in treating or preventing any one of the diseases mentioned hereinbefore.

The invention also relates to the use of a compound of Formula (I), a tautomer or a stereoisomeric form thereof, or a pharmaceutically acceptable salt, or a solvate thereof, for the manufacture of a medicament for the treatment or prevention of any one of the disease conditions mentioned hereinbefore.

The compounds of the present invention can be administered to mammals, preferably humans, for the treatment or prevention of any one of the diseases mentioned hereinbefore.

In view of the utility of the compounds of Formula (I), the tautomers and the stereoisomeric forms thereof, and the pharmaceutically acceptable salts, and the solvates thereof, there is provided a method of treating warm-blooded animals, including humans, suffering from any one of the diseases mentioned hereinbefore.

Said method comprises the administration, i.e. the systemic or topical administration, of a therapeutically effective amount of a compound of Formula (I), a tautomer or a stereoisomeric form thereof, or a pharmaceutically acceptable salt, or a solvate thereof, to warm-blooded animals, including humans.

Therefore, the invention also relates to a method for the treatment or prevention of any one of the diseases mentioned hereinbefore comprising administering a therapeutically effective amount of compound according to the invention to a patient in need thereof.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. An effective therapeutic daily amount would be from about 0.005 mg/kg to 100 mg/kg. The amount of a compound according to the present invention, also referred to herein as the active ingredient, which is required to achieve a therapeutically effect may vary on case-by-case basis, for example with the particular compound, the route of administration, the age and condition of the recipient, and the particular disorder or disease being treated. A method of treatment may also include administering the active ingredient on a regimen of between one and four intakes per day. In these methods of treatment the compounds according to the invention are preferably formulated prior to administration.

The present invention also provides compositions for preventing or treating the disorders referred to herein. Said compositions comprising a therapeutically effective amount of a compound of Formula (I), a tautomer or a stereoisomeric form thereof, or a pharmaceutically acceptable salt, or a solvate thereof, and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions may be prepared by any methods well known in the art of pharmacy, for example, using methods such as those described in Gennaro et al. Remington's Pharmaceutical Sciences (18^{th}ed., Mack Publishing Company, 1990, see especially Part 8 Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, an embodiment of the present invention relates to a product containing as first active ingredient a compound according to the invention and as further active ingredient one or more anticancer agent, as a combined preparation for simultaneous, separate or sequential use in the treatment of patients suffering from cancer.

The one or more other medicinal agents and the compound according to the present invention may be administered simultaneously (e.g. in separate or unitary compositions) or sequentially in either order. In the latter case, the two or more compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular other medicinal agent and compound of the present invention being administered, their route of administration, the particular condition, in particular tumour, being treated and the particular host being treated.

The following examples further illustrate the present invention.

### Examples

Several methods for preparing the compounds of this invention are illustrated in the following examples. Unless otherwise noted, all starting materials were obtained from commercial suppliers and used without further purification, or alternatively can be synthesized by a skilled person by using well-known methods.

| Abbreviation | Meaning |
|---|---|
| CH₃COONH₄ | ammonium acetate |
| 1,2-DCE | 1,2-dichloroethane |
| sat. or Sat. | saturated |
| ° C. | degree Celsius |
| AcOH or CH₃COOH | acetic acid |
| Ac₂O | acetic anhydride |
| aq. | aqueous |
| atm | atmosphere |
| BH3•THF | Borane tetrahydrofuran complex |
| Boc or boc | tert-butyloxycarbonyl |
| BOC-anhydride | di-tert-butyl dicarbonate |
| BPin or PinB | 4,4,5,5-tetramethyl-1,3,2-dioxaborolane |
| Celite | diatomaceous earth |
| CO₂ | carbon dioxide |
| Cs₂CO₃ | cesium carbonate |
| CPME | cyclopentyl methylether |
| DCM or CH2Cl2 | dichloromethane |
| DEA | diethanolamine |
| DEE | diethyl ether |
| DIEA or DIPEA | N-ethyl-N-(propan-2-yl)propan-2-amine |
| DMA | N,N-dimethylacetamide |
| DME | 1,2-dimethoxyethane |
| DMF | N,N-dimethylformamide |
| DMSO | (methanesulfinyl)methane |
| DSC | differential scanning calorimetry |
| EDCI or EDCI•HCl | 3-{[(ethylimino)methylidene]amino}-N,N- |
| | dimethylpropan-1-amine |
| ee | enantiomeric excess |
| ESI | electrospray ionization |
| EtOAc or EA | ethyl acetate |
| EtOH | ethanol |
| FA | formic acid |
| FCC | flash column chromatography |
| h or hr | hour(s) |
| HATU | 1-[bis(dimethylamino)methylene]-1H- |
| | 1,2,3-triazolo[4,5-b]pyridinium 3-oxide |
| | hexafluorophosphate |
| HCl | hydrochloric acid |
| Hex | hexane |
| HOBt | 1-hydroxybenzotriazole |
| IBX | 2-iodoxybenzoic acid |
| Insolute ® SCX-3 | ethylbenzene sulfonic acid cation |
| | exchange resin |
| Ir[dF(CF₃)ppy]₂(dtbpy))PF6 | [4,4′-Bis(1,1-dimethylethyl)-2,2′- |
| | bipyridine-N1,N1′]bis[3,5-difluoro- |
| | 2-[5-(trifluoromethyl)-2- |
| | pyridinyl-N]phenyl-C]Iridium(III) |
| | hexafluorophosphate |
| i-PrNH₂ or iPrNH | isopropyl amine |
| i-PrOH, iPrOH or IPA | isopropyl alcohol |
| IPAC | isopropyl acetate |
| K₂CO₃ | potassium carbonate |
| KOAc | potassium acetate |
| LCMS | liquid chromatography-mass spectrometry |
| LiAlH₄ | lithium aluminium hydride |
| Li-HMDS or LiHMDS | lithium bis(trimethylsilyl)amide |
| M or N | mol/L |
| MeCN or CH₃CN | acetonitrile |
| MeI | iodomethane |
| MeOH | methanol |
| mg | milligram |
| min | minute(s) |
| mL | milliliter |
| mmol | millimole |
| m-CPBA | meta-chloroperoxybenzoic acid |
| MS | mass spectrometry |
| N₂ | nitrogen |
| Na₂CO₃ | sodium carbonate |
| Na₂SO₄ | sodium sulfate |
| NaBH(OAc)₃ | sodium triacetoxyborohydride |
| NaBH₃CN | sodium cyanoborohydride |
| Na₄EDTA | Ethylenediaminetetraacetic acid |
| | tetrasodium salt |
| NaHCO₃ | sodium hydrogencarbonate |
| NaOAc | sodium acetate |
| NaOH | sodium hydroxide |
| NH₃ | ammonia |
| NH₃•H₂O or NH₄OH | ammonium hydroxide |
| NH₄HCO₃ | ammonium hydrogencarbonate |
| n-BuLi | n-butyllithium |
| NBS | N-Bromosuccinimide |
| NH₄Cl | ammonium chloride |
| NMP | 1-methyl-2-pyrrolidinone |
| NMR | nuclear magnetic resonance |
| Pd(dppf)Cl₂ | [1,1′- |
| | bis(diphenylphosphino)ferrocene] |
| | dichloropalladium(II) |
| Pd(PPh₃)₄ | tetrakis(triphenylphosphine)palladium(0) |
| Pd/C | palladium on carbon |
| PE | petroleum ether |
| PIDA | Diacetoxy iodobenze or iodobenzen |
| | diacetate |
| Prep. HPLC | preparative high-performance liquid |
| | chromatography |
| Prep. SFC | preparative supercritical fluid |
| | chromatography |
| Prep. TLC | preparative thin-layer chromatography |
| Rf | retention factor |
| Rh₂(OAc)₄ | rhodium acetate |
| RP | Reverse(d) phase |
| rt, r.t. or RT | room temperature |
| RuPhos Pd G4/ | Palladium, [[2′,6′-bis(1-methylethoxy)[1,1′- |
| 4^{th} generation RuPhos Pd | biphenyl]-2-yl]dicyclohexylphosphine- |
| precatalyst | κP](methanesulfonato-κO)[2′-(methyl- |
| | amino-κN)[1,1′-biphenyl]-2-yl-κC]-, |
| | (SP-4-3)-(ACI) |
| | CAS 1599466-85-9 |
| sat | saturated |
| SFC | supercritical fluid chromatography |
| SiliaBond ® | Propylsulfonic acid bound to silica |
| propylsulfonic | stationary phase support |
| acid resin | |
| SiliaMetS ® | N1-propylethane-1,2-diamine bound to |
| Diamine | silica stationary phase support |
| t-butyl | tert-butyl |
| t-BuOK | potassium tert-butoxide |
| T3P | propylphosphonic anhydride |
| TEA or Et3N | triethylamine |
| Temp | temperature |
| TFA | trifluo acetic acid |
| THF | tetrahydrofuran |
| TLC | thin-layer chromatography |
| Tonset | Temperature at which melting onset occurs |
| | (measure by DSC) |
| Ts | tosyl |
| UV | ultraviolet |
| v/v | volume to volume |
| w/v | weight to volume |
| w/w | weight to weight |
| ZnCl2 | zinc chloride |
| Xantphos | 4,5-bis(diphenylphosphino)-9,9- |
| | dimethylxanthene |

As understood by a person skilled in the art, compounds synthesized using the protocols as indicated may exist as a solvate e.g. hydrate, and/or contain residual solvent or minor impurities. Compounds or intermediates isolated as a salt form, may be integer stoichiomnetric i.e. mono- or di-salts, or of intermediate stoichiometry. When an intermediate or compound in the experimental part below is indicated as ‘HCI salt’ without indication of the number of equivalents of HCl, this means that the number of equivalents of HCl was not determined. The same principle will also apply to all other salt forms referred to in the experimental part, such as e.g. ‘oxalate salt’, ‘HCOOH salt’ (‘formate salt’), or

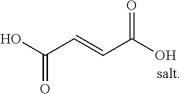

The stereochemical configuration for centers in some compounds may be designated “R” or “S” when the mixture(s) was separated and absolute stereochemistry was known, or when only one enantiomer was obtained and absolute stereochemistry was known; for some compounds, the stereochemical configuration at indicated centers has been designated as “*R” or “*S” when the absolute stereochemistry is undetermined (even if the bonds are drawn stereo specifically) although the compound itself has been isolated as a single stereoisomer and is enantiomerically pure. In case a compound designated as “*R” is converted into another compound, the “*R” indication of the resulting compound is derived from its starting material.

For example, it will be clear that Compound 135

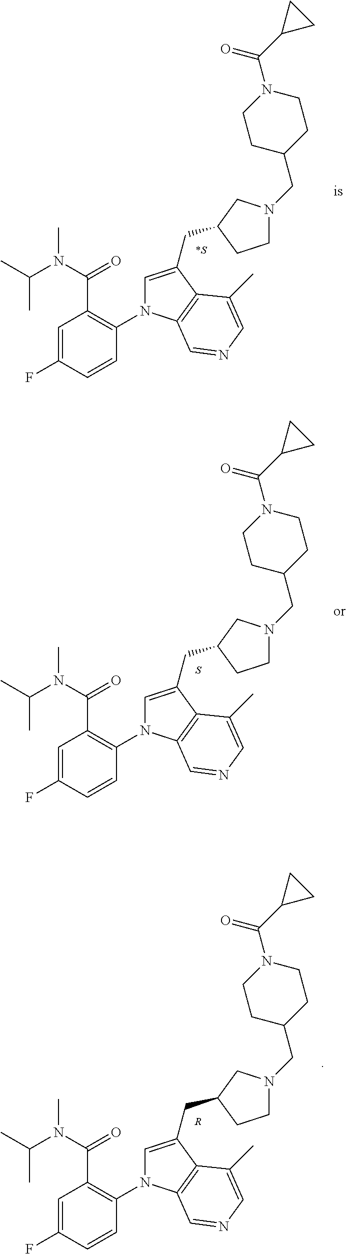

For compounds wherein the stereochemical configuration of two stereocentres is indicated by * (e.g. *R or *S), the absolute stereochemistry of the stereocentres is undetermined (even if the bonds are drawn stereospecifically), although the compound itself has been isolated as a single stereoisomer and is enantiomerically pure. In this case, the configuration of the first stereocentre indicated by * is independent of the configuration of the second stereocentre indicated by * in the same compound. “*R” or “*S” is assigned randomly for such molecules. Similar for compounds wherein the stereochemical configuration of three stereocentres is indicated by * (e.g. *R or *S), the absolute stereochemistry of the stereocentres is undetermined (even if the bonds are drawn stereospecifically), although the compound itself has been isolated as a single stereoisomer and is enantiomerically pure. In this case, the configuration of the stereocentres indicated by * are independent of the configuration of the other stereocentres indicated by * in the same compound. “*R” or “*S” is assigned randomly for such molecules.

For example, for Compound 9b

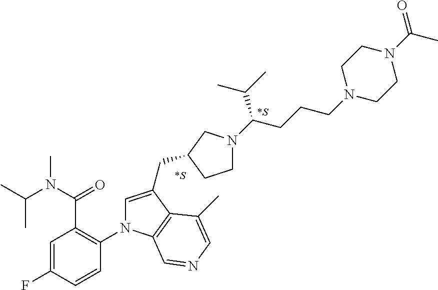 [SMILES: CC(=O)N1CCN(CCC[C@@H](C(C)C)N2CC[C@H](Cc3cn(-c4ccc(F)cc4C(=O)N(C)C(C)C)c4cncc(C)c34)C2)CC1]

this means that the compound is

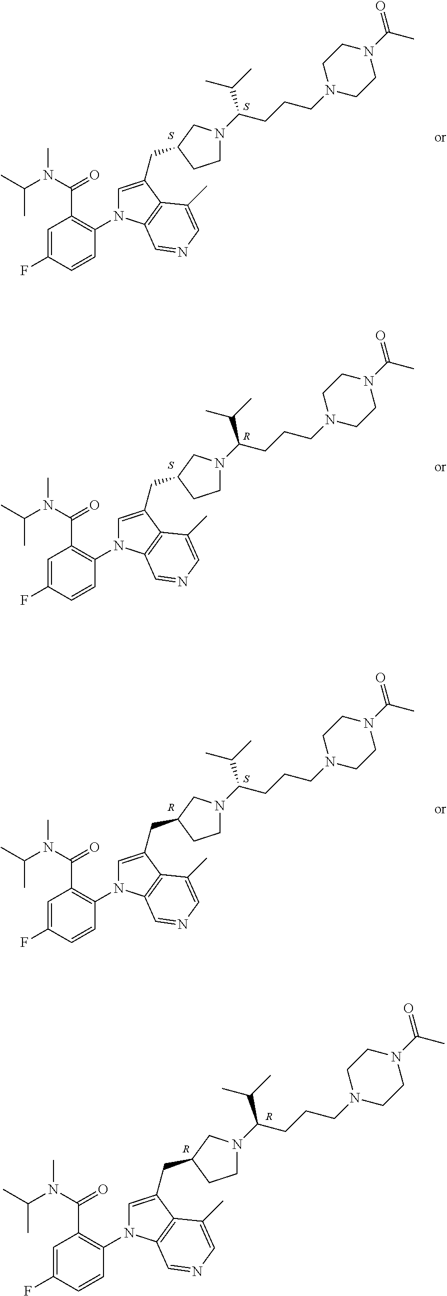

A skilled person will realize that the paragraphs above about stereochemical configurations, also apply to intermediates.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers or undetermined stereochemistry, unless otherwise is indicated or is clear from the context.

When a stereocenter is indicated with ‘RS’ this means that a racemic mixture was obtained at the indicated centre, unless otherwise indicated.

A double bond indicated with EZ means the compound/intermediate was obtained as a mixture of E and Z isomers.

### Preparation of Intermediates and Compounds

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

### Preparation of Intermediate 1:

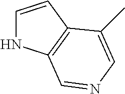

To a solution of 4-bromo-TH-pyrrolo[2,3-c]pyridine (2 g, 95% purity, 9.64 mmol) in 1,4-dioxane (30 mL) and water (4 mL) was added 2,4,6-trimethyl-1,3,5,2,4,6-trioxatriborinane (7.26 g, 50% in THF, 28.9 mmol) and potassium carbonate (4.0 g, 28.9 mmol). The suspension was degassed and exchanged with N₂ twice. [1,1′-bis(diphenylphosphino)ferrocene]dichloropalladium(II) (706 mg, 0.964 mmol) was added into the reaction mixture. The reaction mixture was heated up to 100° C. and stirred at this temperature overnight. After cooled down to r.t., the reaction mixture was filtered and the filtrate was concentrated. The resulting residue was purified by silica gel column chromatography eluting with ethyl acetate in petroleum ether from 0% to 80% to give intermediate 1 (1.01 g, 95% purity, 75.3% yield).

Alternatively, intermediate 1 can also be prepared with the following procedure:

Into a 20 L 4-necked round-bottom flask were added 4-bromo-1H-pyrrolo[2,3-c]pyridine (1330 g, 6750 mmol, 1.00 equiv), Pd(dppf)Cl₂ (493.9 g, 675 mmol, 0.10 equiv), K₂CO₃ (2798.69 g, 20250.21 mmol, 3.00 equiv), 1,4-dioxane (13 L), H₂O (2 L) and 2,4,6-trimethyl-1,3,5,2,4,6-trioxatriborinane (2542.01 g, 20250.21 mmol, 3.00 equiv) at room temperature. The resulting mixture was stirred for overnight at 100° C. The mixture was allowed to cool down to room temperature. The resulting mixture was concentrated under reduced pressure. The resulting mixture was diluted with water (15 L). The aqueous layer was extracted with EtOAc (3×10 L) and the organic layer was washed with water (2×5 L). The resulting liquid was dried with Na₂SO₄, filtered and concentrated under vacuum. The residue was purified by silica gel column chromatography, eluting with with 10% methanol in dichloromethane to afford intermediate 1 (640 g, yield: 72%) as a grey solid.

### Preparation of Intermediate 2:

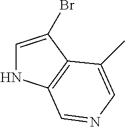

At 0° C., to a solution of intermediate 1 (918 mg, 95% purity, 6.6 mmol) in DMF (60 mL) was added a solution of N-bromosuccinimide (1.17 g, 6.6 mmol) in DMF (10 mL) dropwise. The reaction mixture was stirred at this temperature for 30 minutes. The reaction mixture was quenched with water and extracted with ethyl acetate (50 mL) twice. The organic layer was washed with brine (25 mL), dried over sodium sulfate, filtered and concentrated to afford the crude product, which was purified by silica gel column chromatography eluting with ethyl acetate in petroleum from 0% to 60% to give intermediate 2 (1.14 g, 97.1% purity, 79.5% yield) as a white solid.

Alternatively, intermediate 2 can also be prepared with the following procedure:

Into a 10 L 4-necked round-bottom flask were added intermediate 1 (640 g, 4842.39 mmol, 1.00 equiv) and DMF (5.00 L) at room temperature. To the above mixture was added NBS (861.87 g, 4842.40 mmol, 1.00 equiv) in portions over 1 h at room temperature. The resulting mixture was stirred for additional 30 min at room temperature. The reaction was quenched by the addition of aqueous solution of Na₂S₂O₃ (10 L, 10% (w/v)) at room temperature. The aqueous layer was extracted with EtOAc (3×5 L) and the organic layer was washed with brine (1×5 L). The resulting liquid was dried with Na₂SO₄ and concentrated. The residue was purified by silica gel column chromatography, eluting with 20% ethyl acetate in petroleum ether to afford intermediate 2 (800 g, yield: 78%) as a grey solid.

The Following Intermediates were Synthesized by an Analogous Method as Described for Intermediate 2

| Int. No. | Structure | Starting Materials |
|---|---|---|
| Intermediate 3 | 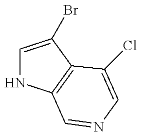 [SMILES: Clc1cncc2[nH]cc(Br)c12] | 4-chloro-1H- pyrrolo[2,3-c]pyridine |
| Intermediate 350 | 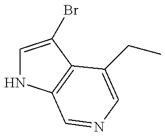 | 4-ethyl-1H- pyrrolo[2,3-c]pyridine |

### Preparation of Intermediate 4:

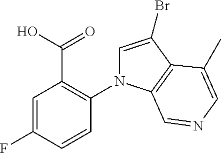 [SMILES: Cc1cncc2c1c(Br)cn2-c1ccc(F)cc1C(=O)O]

To a solution of intermediate 2 (1.14 g, 97.1% purity, 5.24 mmol) in DMF (80 mL) were added 5-fluoro-2-iodobenzoic acid (1.40 mg, 5.24 mmol), copper powder (333 mg, 5.24 mmol) and potassium carbonate (2.18 g, 15.7 mmol). The reaction mixture was heated up to 100° C. and stirred at this temperature overnight. After the mixture was cooled down to r.t., the reaction mixture was concentrated and the resulting residue was acidified with HCl (1 N) to pH=˜3. The resulting mixture was filtered and the filter cake was washed with water twice. The filter cake was dried under vacuum to give crude intermediate 4 (1.8 g, 91% purity, 89.4% yield) as a yellow solid.

Alternatively, intermediate 4 can also be prepared with the following procedure:

Into a 10 L 4-necked round-bottom flask were added intermediate 2 (560 g, 2653.24 mmol, 1.00 equiv), Cu (252.91 g, 3979.87 mmol, 1.50 equiv), K₂CO₃ (1100.08 g, 7959.74 mmol, 3.00 equiv) and 5-fluoro-2-iodobenzoic acid (705.79 g, 2653.24 mmol, 1.00 equiv) in DMF (6.00 L) at room temperature. The resulting mixture was stirred for additional 2 h at 100° C. under nitrogen atmosphere. The resulting mixture was filtered, the filter cake was washed with DMF (1×5 L) and the filtrate was concentrated under reduced pressure. The resulting mixture was diluted with water (8 L). The mixture was acidified to pH 3 with aqueous HCl (conc.). The precipitated solids were collected by filtration and washed with water (3×3 L). The resulting solid was dried under vacuum to afford intermediate 4 (1300 g, crude) as a grey solid.

The Following Intermediates were Synthesized by an Analogous Method as Described for Intermediate 4

| Int. | | Starting |
|---|---|---|
| No. | Structure | Materials |
| Intermediate 5 | 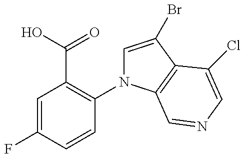 [SMILES: O=C(O)c1cc(F)ccc1-n1cc(Br)c2c(Cl)cncc21] | Intermediate 3 |
| Intermediate 110 | 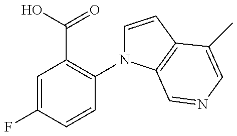 [SMILES: Cc1cncc2c1ccn2-c1ccc(F)cc1C(=O)O] | Intermediate 1 |
| Intermediate 351 | 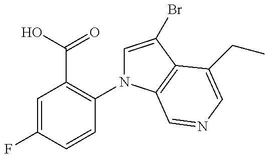 [SMILES: CCc1cncc2c1c(Br)cn2-c1ccc(F)cc1C(=O)O] | Intermediate 350 |

### Preparation of Intermediate 6:

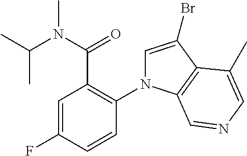 [SMILES: Cc1cncc2c1c(Br)cn2-c1ccc(F)cc1C(=O)N(C)C(C)C]

At 0° C., to a solution of intermediate 4 (1.8 g, 91% purity, 4.69 mmol) in DMF (50 mL) was added HATU (4.46 g, 11.7 mmol), N,N-diisopropylethylamine (3.03 g, 23.5 mmol) and N-methylpropan-2-amine (858 mg, 11.7 mmol). After addition, the mixture was stirred at room temperature overnight. The reaction mixture was concentrated and the resulting residue was purified by silica gel column chromatography eluted with methanol in dichloromethane from 0% to 5% to give intermediate 6 (2.0 g, 93% purity, 98.1% yield) as a yellow oil.

Alternatively, intermediate 6 can also be prepared with the following procedure:

Into a 20 L 4-necked round-bottom flask were added intermediate 4 (920 g, 2634.90 mmol, 1.00 equiv, same as 1300 g crude), DMF (7.5 L), HATU (1102.06 g, 2898.39 mmol, 1.10 equiv) and DIEA (1021.63 g, 7904.70 mmol, 3.00 equiv) at room temperature. The resulting mixture was stirred for additional 30 min at room temperature. To the above mixture was added N-methylpropan-2-amine (211.99 g, 2898.39 mmol, 1.10 equiv) dropwise over 10 min at 0° C. The resulting mixture was stirred overnight at room temperature. The reaction was quenched by the addition of water (20 L) at room temperature. The aqueous layer was extracted with EtOAc (3×7 L) and the organic layer was washed with water (3×5 L). The resulting liquid was dried with Na₂SO₄, filtered and concentrated under reduced pressure. The residue was purified by silica gel column chromatography, eluting with 50% ethyl acetate in petroleum ether (1:1) to afford intermediate 6 (700 g, yield: 66%) as a light yellow solid.

### Alternative Approach for the Preparation of Intermediate 6

Intermediate 111 (1.3 g, 4.0 mmol) was dissolved in MeCN (40 mL). Next, CuBr₂ (2.7 g, 12 mmol) was added, and the mixture was stirred at room temperature for 5 h. Next, 7N NH₃/MeOH (20 mL) was added. The reaction mixture was stirred vigorously for −30 min. Then, water (40 mL) and isopropyl acetate were added. The layers were separated, and the water layer was extracted twice with isopropyl acetate. The organic layers were combined, washed with brine, dried over Na₂SO₄, filtered and evaporated to dryness. The residue was purified by silica gel column chromatography eluting with methanol in dichloromethane from 0% to 3% to provide intermediate 6 (1.2 g, yield 72%) as an orange oil.

The Following Intermediates were Synthesized by an Analogous Method as Described for Intermediate 6

| Int. No. | Structure | Starting Materials |
|---|---|---|
| Intermediate 7 | 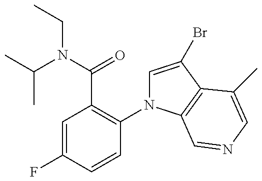 [SMILES: CCN(C(=O)c1cc(F)ccc1-n1cc(Br)c2c(C)cncc21)C(C)C] | Intermediate 4 & N-ethylpropan-2-amine |
| Intermediate 8 | 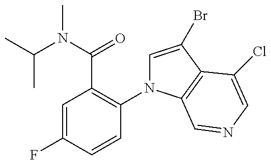 [SMILES: CC(C)N(C)C(=O)c1cc(F)ccc1-n1cc(Br)c2c(Cl)cncc21] | Intermediate 5 & N-methylpropan-2-amine |
| Intermediate 111 | 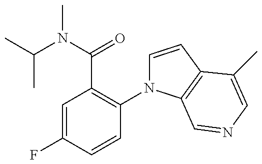 [SMILES: Cc1cncc2c1ccn2-c1ccc(F)cc1C(=O)N(C)C(C)C] | Intermediate 110 & N-methylpropan-2-amine |
| Intermediate 317 | 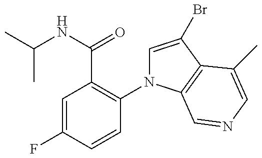 [SMILES: Cc1cncc2c1c(Br)cn2-c1ccc(F)cc1C(=O)NC(C)C] | Intermediate 4 & propan-2-amin |
| Intermediate 345 | 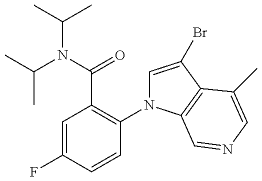 [SMILES: Cc1cncc2c1c(Br)cn2-c1ccc(F)cc1C(=O)N(C(C)C)C(C)C] | Intermediate 4 & diisopropylamine |
| Intermediate 352 | 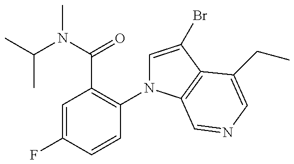 [SMILES: CCc1cncc2c1c(Br)cn2-c1ccc(F)cc1C(=O)N(C)C(C)C] | Intermediate 351 & N- methylpropan-2-amine |

### Preparation of Intermediate 9:

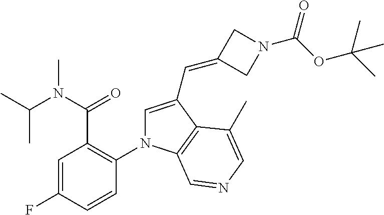 [SMILES: Cc1cncc2c1c(C=C1CN(C(=O)OC(C)(C)C)C1)cn2-c1ccc(F)cc1C(=O)N(C)C(C)C]

To a mixture of intermediate 6 (4 g, 4.312 mmol), tert-butyl 3-((4,4,5,5-tetramethyl-1,3,2-dioxaborolan-2-yl)methylene)azetidine-1-carboxylate (2.92 g, 9.9 mmol) and potassium carbonate (2.7 g, 19.7 mmol) in 1,4-dioxane (70 mL) and water (23 mL) was added Pd(dppf)Cl₂ (724 mg, 0.99 mmol). The mixture was degassed under nitrogen atmosphere three times and the reaction was stirred at 100° C. under nitrogen atmosphere for 16 h. After the mixture was cooled down to RT, the reaction mixture was diluted with H₂O and extracted with EtOAc. The combined organic phase was washed with brine, dried over Na₂SO₄, filtered and concentrated under vacuum. The residue was purified by silica gel column chromatography eluting with 90% ethyl acetate in petroleum ether to give intermediate 9 (1.8 g, 45.7% purity, 38.7% yield) as a yellow solid.

### Preparation of Intermediate 10:

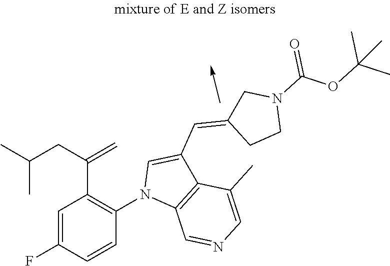

A mixture intermediate 6 (12.0 g, 29.8 mmol), tert-butyl 3-((4,4,5,5-tetramethyl-1,3,2-dioxaborolan-2-yl)methylene)pyrrolidine-1-carboxylate (9.2 g, 29.8 mmol) and potassium carbonate (12.3 g, 89.1 mmol) in 1,4-dioxane (120 mL) and water (20 mL) was degassed and exchanged with N₂ twice. Pd(dppf)Cl₂ (2.16 g, 2.95 mmol) was added and the reaction mixture was heated up to 100° C. and stirred at this temperature overnight. After the reaction mixture was cooled down to r.t., the resulting mixture was concentrated and the residue was purified by silica gel column chromatography eluting with ethyl acetate in petroleum ether from 0% to 80% to give intermediate 10 (12.0 g, 79.4% yield) as a yellow oil.

The Following Intermediates were Synthesized by an Analogous Method as Described for Intermediate 10

| Int. No. | Structure | Starting Materials |
|---|---|---|
| Intermediate 11 | 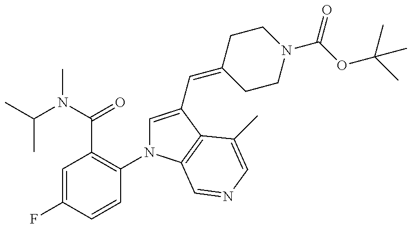 [SMILES: Cc1cncc2c1c(C=C1CCN(C(=O)OC(C)(C)C)CC1)cn2-c1ccc(F)cc1C(=O)N(C)C(C)C] | Intermediate 6 & tert-butyl 4- ((4,4,5,5-tetramethyl-1,3,2- dioxaborolan-2- yl)methylene)piperidine-1- carboxylate |
| Intermediate 12 | 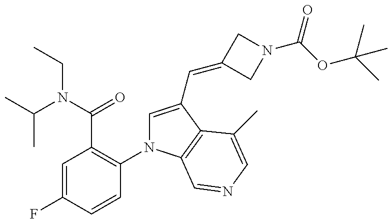 [SMILES: CCN(C(=O)c1cc(F)ccc1-n1cc(C=C2CN(C(=O)OC(C)(C)C)C2)c2c(C)cncc21)C(C)C] | Intermediate 7 & tert-butyl 3- ((4,4,5,5-tetramethyl-1,3,2- dioxaborolan-2- yl)methylene)azetidine-1- carboxylate |
| Intermediate 13 | 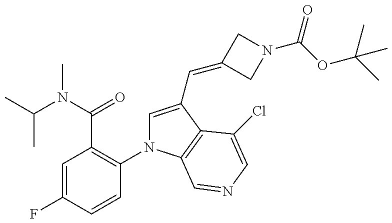 [SMILES: CC(C)N(C)C(=O)c1cc(F)ccc1-n1cc(C=C2CN(C(=O)OC(C)(C)C)C2)c2c(Cl)cncc21] | Intermediate 8 & tert-butyl 3- ((4,4,5,5-tetramethyl-1,3,2- dioxaborolan-2- yl)methylene)azetidine-1- carboxylate |
| Intermediate 14 | 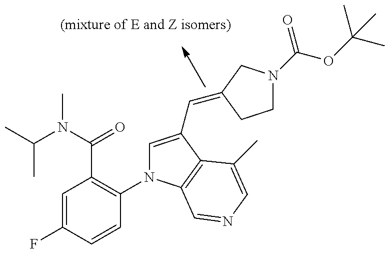 [SMILES: Cc1cncc2c1c(/C=C1\CCN(C(=O)OC(C)(C)C)C1)cn2-c1ccc(F)cc1C(=O)N(C)C(C)C] | Intermediate 8 & tert-butyl-3- ((4,4,5,5-tetramethyl-1,3,2- dioxaborolan-2- yl)methylene)pyrrolidine-1- carboxylate (mixture of E and Z isomers) |
| Intermediate 362 | 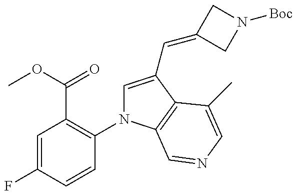 [SMILES: COC(=O)c1cc(F)ccc1-n1cc(C=C2CN(C(=O)OC(C)(C)C)C2)c2c(C)cncc21] | Intermediate 361 & tert-butyl 3- ((4,4,5,5-tetramethyl-1,3,2- dioxaborolan-2- yl)methylene)azetidine-1- carboxylate |

### Preparation of Intermediate 15:

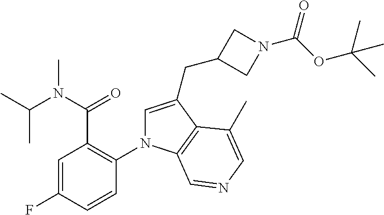 [SMILES: Cc1cncc2c1c(CC1CN(C(=O)OC(C)(C)C)C1)cn2-c1ccc(F)cc1C(=O)N(C)C(C)C]

A mixture of intermediate 9 (6.0 g, 12.2 mmol) in methanol (100 mL) was degassed under nitrogen atmosphere three times. 10 w/w % palladium on charcoal (3 g) was added and the mixture was degassed under hydrogen atmosphere three times. The mixture was stirred at r.t. under hydrogen atmosphere (balloon) for 16 h. The mixture was filtered and the filtrate was concentrated and purified by silica gel column chromatography eluting with 50% ethyl acetate in petroleum ether to give intermediate 15 (5.2 g, 97% purity, 83.7% yield) as a yellow solid.

The Following Intermediates were Synthesized by an Analogous Method as Described for Intermediate 15

| Int. No. | Structure | Starting Materials |
|---|---|---|
| Intermediate 19 | 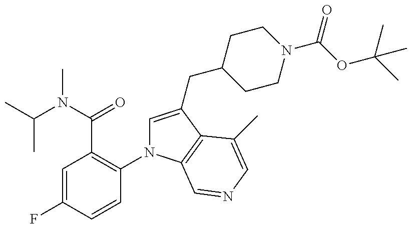 [SMILES: Cc1cncc2c1c(CC1CCN(C(=O)OC(C)(C)C)CC1)cn2-c1ccc(F)cc1C(=O)N(C)C(C)C] | Intermediate 11 |
| Intermediate 20 | 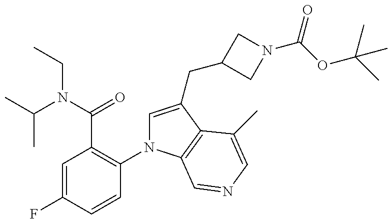 [SMILES: CCN(C(=O)c1cc(F)ccc1-n1cc(CC2CN(C(=O)OC(C)(C)C)C2)c2c(C)cncc21)C(C)C] | Intermediate 12 |
| Intermediate 21 | 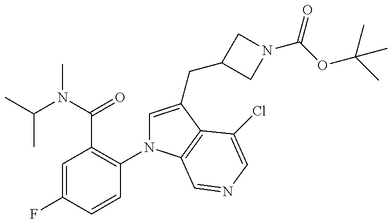 [SMILES: CC(C)N(C)C(=O)c1cc(F)ccc1-n1cc(CC2CN(C(=O)OC(C)(C)C)C2)c2c(Cl)cncc21] | Intermediate 13 |
| Intermediate 22 | 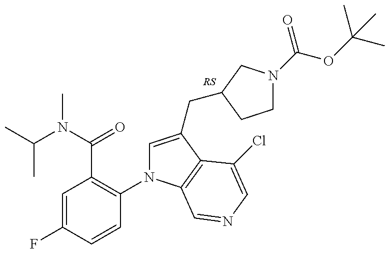 [SMILES: CC(C)N(C)C(=O)c1cc(F)ccc1-n1cc(CC2CCN(C(=O)OC(C)(C)C)C2)c2c(Cl)cncc21] | Intermediate 14 |
| Intermediate 363 | 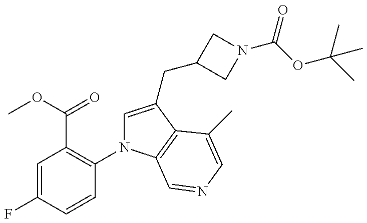 [SMILES: COC(=O)c1cc(F)ccc1-n1cc(CC2CN(C(=O)OC(C)(C)C)C2)c2c(C)cncc21] | Intermediate 362 |

### Preparation of Intermediate 16, 17 & 18:

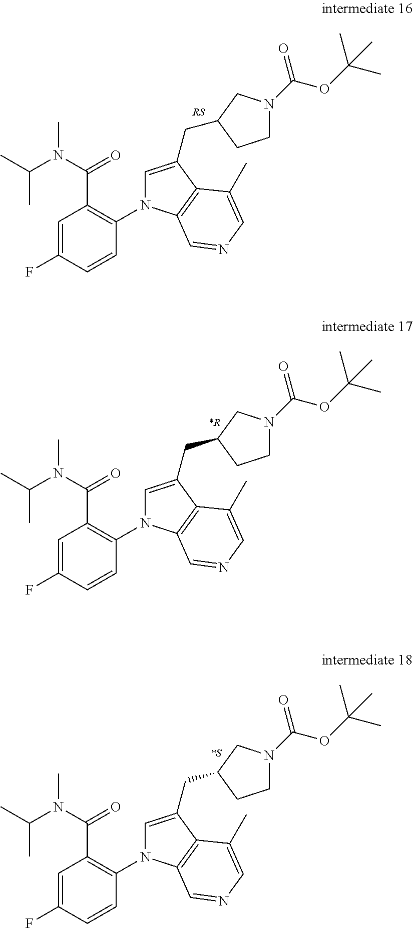

To a solution of intermediate 10 (2.5 g, 93% purity, 4.59 mmol) in methanol (40 mL) was added w/w % palladium on charcoal (1 g) under N₂. The suspension was degassed under vacuum and purged with H₂ several times. The reaction mixture was heated up to 30° C. and stirred at this temperature overnight. After the reaction was cooled down to r.t., the reaction mixture was filtered and the filtrate was concentrated and purified by silica gel column chromatography eluted with methanol in dichloromethane from 0% to 5% to give intermediate 16 (2.5 g, 93% purity, 99.6% yield) as a yellow oil.

Intermediate 16 (8 g, 95% purity, 14.9 mmol) was separated by chiral IG-SFC (separation condition: Column: IG; Mobile Phase: CO₂-IPA: 65:35, at 60 mL/min; Temp: 40° C.; Wavelength: 214 nm) to afford intermediate 17 (first fraction, 3.29 g, 98% purity, 42.4% yield) as a yellow oil and intermediate 18 (second fraction, 3.36 g, 98% purity, 43.3% yield) as a yellow solid.

Chiral SFC method 2 was employed to match the stereochemistry of intermediate 18 and intermediate 201, retention time=5.97-6.10 min.

### Preparation of Intermediate 23 & 24:

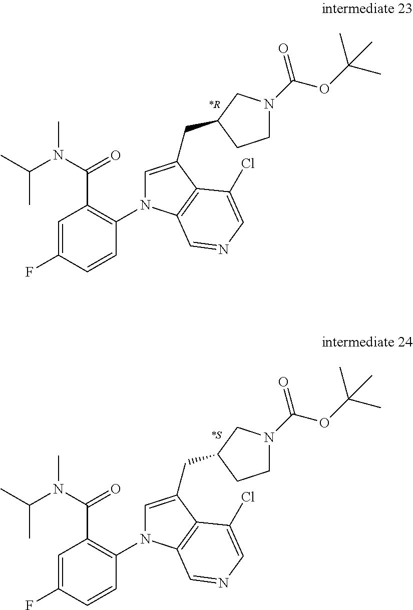

Intermediate 22 (1.40 g, 2.65 mmol) was separated by SFC (DAICEL CHIRALPAK IG (250 mm*50 mm, 10 um)); Mobile phase: A: Supercritical CO₂, B: 0.1% NH₃H₂O IPA; Isocratic: A:B=55:45; Flow rate: 200 mL/min) to afford two fractions. The first fraction was collected as intermediate 23 (620 mg, 98.6% purity, 44% yield) as yellow solid. The second fraction was collected as intermediate 24 (650 mg, 99.9% purity, 46% yield) as a yellow solid.

### Preparation of Intermediate 25:

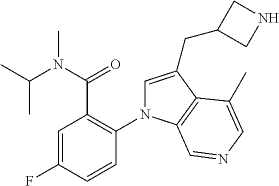 [SMILES: Cc1cncc2c1c(CC1CNC1)cn2-c1ccc(F)cc1C(=O)N(C)C(C)C]

To a cooled (ice bath) solution of intermediate 15 (1.1 g, 2.2 mmol) in dichloromethane (14 mL) was added dropwise TFA (7 mL). Then, the mixture was stirred at r.t. for 2 h. The solvent was removed by evaporation and the residue was dissolved in DCM, the pH was adjusted to 8-9 with saturated sodium carbonate aqueous solution, and extracted with DCM. The organic phase was dried over Na₂SO₄ and concentrated under vacuum to give intermediate 25 (680 mg, 72% yield) as a white solid.

The Following Intermediates and Compounds were Synthesized by an Analogous Method as Described for Intermediate 25

| | | Starting Materials & |
|---|---|---|
| Int./Co No. | Structure | Methods |
| Intermediate 26 | 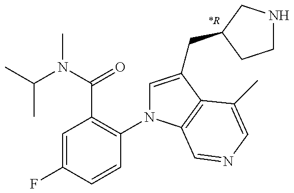 [SMILES: Cc1cncc2c1c(C[C@@H]1CCNC1)cn2-c1ccc(F)cc1C(=O)N(C)C(C)C] | Intermediate 17 |
| Intermediate 27 | 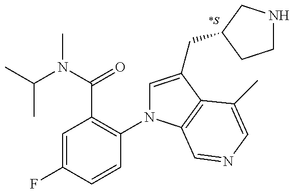 [SMILES: Cc1cncc2c1c(C[C@H]1CCNC1)cn2-c1ccc(F)cc1C(=O)N(C)C(C)C] | Intermediate 18 |
| Intermediate 28 | 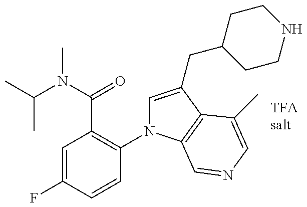 [SMILES: Cc1cncc2c1c(CC1CCNCC1)cn2-c1ccc(F)cc1C(=O)N(C)C(C)C] | Intermediate 19 The reaction mixture was concentrated to afford TFA salt |
| Intermediate 29 | 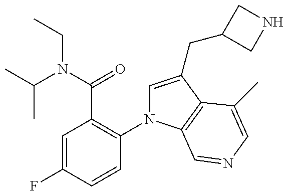 [SMILES: CCN(C(=O)c1cc(F)ccc1-n1cc(CC2CNC2)c2c(C)cncc21)C(C)C] | Intermediate 20 |
| Intermediate 30 | 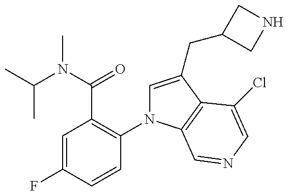 [SMILES: CC(C)N(C)C(=O)c1cc(F)ccc1-n1cc(CC2CNC2)c2c(Cl)cncc21] | Intermediate 21 |
| Intermediate 31 | 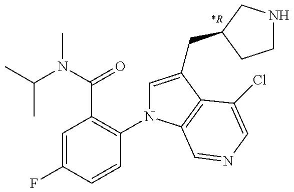 [SMILES: CC(C)N(C)C(=O)c1cc(F)ccc1-n1cc(C[C@@H]2CCNC2)c2c(Cl)cncc21] | Intermediate 23 |
| Intermediate 32 | 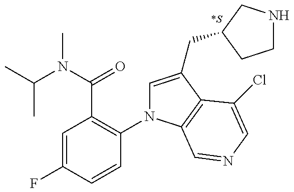 [SMILES: CC(C)N(C)C(=O)c1cc(F)ccc1-n1cc(C[C@H]2CCNC2)c2c(Cl)cncc21] | Intermediate 24 |
| Compound 503 | 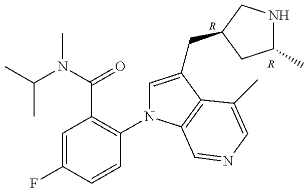 | Intermediate 184 |
| Compound 504 | 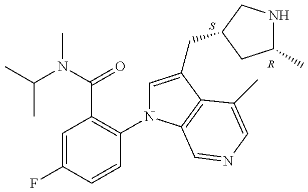 | Intermediate 185 |
| Compound 505 | 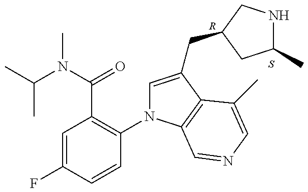 | Intermediate 186 |
| Compound 522 | 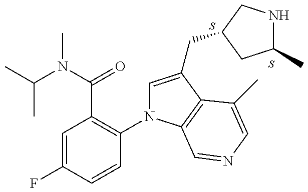 | Intermediate 187 |
| Compound 506 | 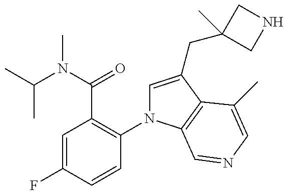 [SMILES: Cc1cncc2c1c(CC1(C)CNC1)cn2-c1ccc(F)cc1C(=O)N(C)C(C)C] | Intermediate 203 |
| Intermediate 213 | 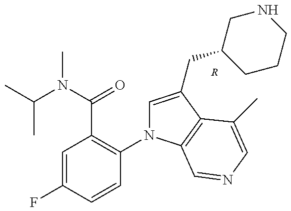 | Intermediate 212 |
| Intermediate 215 | 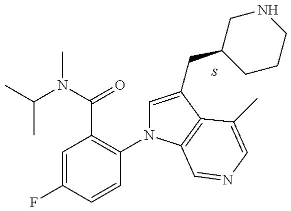 | Intermediate 214 |
| Intermediate 231 | 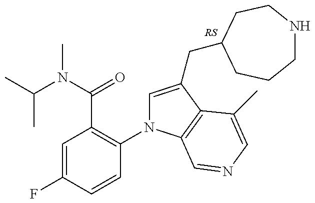 [SMILES: Cc1cncc2c1c(CC1CCCNCC1)cn2-c1ccc(F)cc1C(=O)N(C)C(C)C] | Intermediate 230 |
| Intermediate 236 | 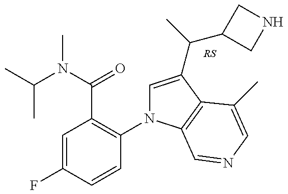 [SMILES: Cc1cncc2c1c(C(C)C1CNC1)cn2-c1ccc(F)cc1C(=O)N(C)C(C)C] | Intermediate 235 |
| Compound 507 | 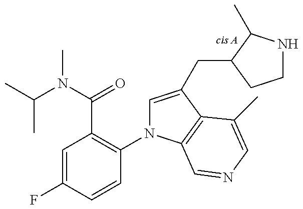 [SMILES: Cc1cncc2c1c(CC1CCNC1C)cn2-c1ccc(F)cc1C(=O)N(C)C(C)C] | Intermediate 303a |
| Compound 508 | 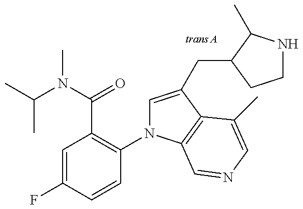 [SMILES: Cc1cncc2c1c(CC1CCNC1C)cn2-c1ccc(F)cc1C(=O)N(C)C(C)C] | Intermediate 303b |
| Compound 509 | 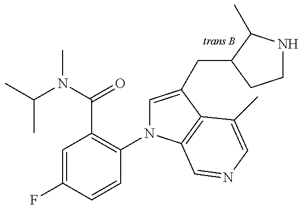 [SMILES: Cc1cncc2c1c(CC1CCNC1C)cn2-c1ccc(F)cc1C(=O)N(C)C(C)C] | Intermediate 304a |
| Compound 510 | 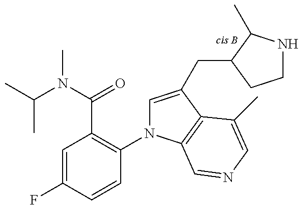 [SMILES: Cc1cncc2c1c(CC1CCNC1C)cn2-c1ccc(F)cc1C(=O)N(C)C(C)C] | Intermediate 304b |
| Intermediate 319 | 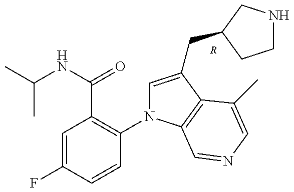 | Intermediate 318 |
| Compound 511 | 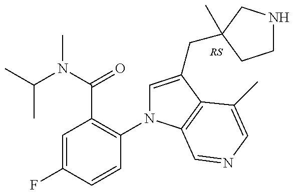 | Intermediate 322 |
| Intermediate 338 | 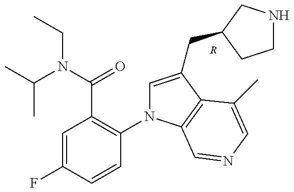 | Intermediate 337 |
| Intermediate 342 | 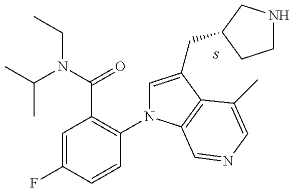 | Intermediate 341 |
| Intermediate 347 | 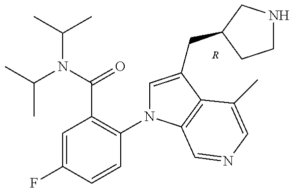 | Intermediate 346 |
| Intermediate 354 | 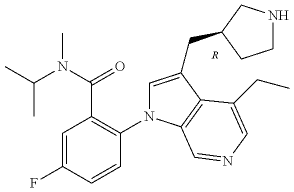 | Intermediate 353 |
| Intermediate 358 | 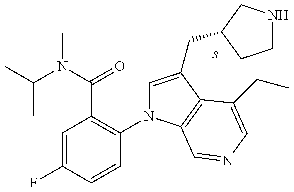 | Intermediate 357 |
| Intermediate 364 | 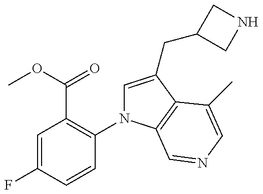 [SMILES: COC(=O)c1cc(F)ccc1-n1cc(CC2CNC2)c2c(C)cncc21] | Intermediate 363 |
| Intermediate 399 | 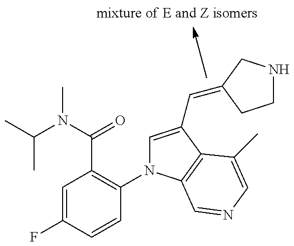 [SMILES: Cc1cncc2c1c(/C=C1\CCNC1)cn2-c1ccc(F)cc1C(=O)N(C)C(C)C] | Intermediate 10 |

### Preparation of Intermediate 33:

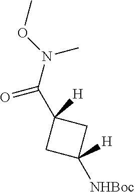 [SMILES: CON(C)C(=O)[C@H]1C[C@@H](NC(=O)OC(C)(C)C)C1]

To a solution of cis-3-[[(1,1-dimethylethoxy)carbonyl]amino]-cyclobutanecarboxylic acid (10.0 g, 46.5 mmol) in DMF (100 mL) was added HOBt (8.15 g, 60.3 mmol), EDCI (11.6 g, 60.5 mmol) and DIEA (30.0 mL, 182 mmol) at 0° C. Then N,O-dimethylhydroxylamine hydrochloride (5.90 g, 60.5 mmol) was added at 0° C. The mixture was stirred at room temperature for 16 hours. The mixture was diluted with ethyl acetate (500 mL), washed with 1 M aq. HCl solution (150 mL), saturated aq. NaHCO₃ solution (100 mL×2) and brine (300 mL×3), dried over Na₂SO₄, filtered and concentrated under reduced pressure to give intermediate 33 (11.0 g, crude) as a white solid, which was used in the next step without further purification.

Preparation of intermediate 34:

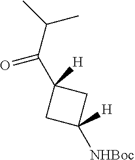 [SMILES: CC(C)C(=O)[C@H]1C[C@@H](NC(=O)OC(C)(C)C)C1]

To a solution of intermediate 33 (11.0 g, 6.97 mmol) in THF (100 mL) was added isopropylmagnesium chloride (64.0 mL, 128 mmol, 2M in THF) dropwise at 0° C. under N₂ atmosphere. The mixture was stirred at room temperature for 12 hours under N₂ atmosphere. The mixture was quenched with saturated aq. NH₄Cl solution (100 mL). The mixture was filtered through a pad of Celite® and the filtrate was extracted with ethyl acetate (200 mL×2). The combined organic layers were washed with brine (200 mL×2), dried over Na₂SO₄, filtered and concentrated under reduced pressure. The crude product was purified by silica gel column chromatography eluting with ethyl acetate in petroleum ether from 0% to 15% to yield intermediate 34 (6.30 g) as a white solid.

### Preparation of Intermediate 35:

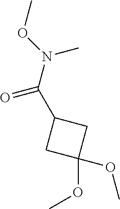

To a solution of 3,3-dimethoxycyclobutanecarboxylic acid (12.0 g, 75 mmol) in DCM (145 mL) was added T₃P (100 mL, 168 mmol. 50% in EtOAc) and DIEA (64 mL, 372 mmol) at 0° C. Then N,O-dimethylhydroxylamine hydrochloride (8.8 g, 89.5 mmol) was added at 0° C. The mixture was stirred at room temperature for 16 hours. The mixture was poured into a saturated solution of NaHCO₃ and EtOAc was added. The organic layer was separated, washed with brine, dried over MgSO₄, filtered and concentrated under reduced pressure to give intermediate 35 (16.0 g, crude) which was used in the next step without further purification.

### Preparation of Intermediate 36:

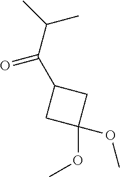

To a solution of intermediate 35 (15.7 g, 77.7 mmol) in THF (420 mL) was added isopropylmagnesium chloride (178.5 mL, 232 mmol, 2M in THF) dropwise at 0° C. under N₂ atmosphere. The reaction mixtures were stirred at room temperature for 12 hours under N₂ atmosphere. The reaction was performed twice on 15.7 g of intermediate 35 and respective reaction media were mixed for the work-up and purification. The combined reaction mixture was poured into ice-water and a 10% aqueous solution of NH₄Cl and extracted with EtOAc. The organic layer was washed with brine, dried over MgSO₄, filtered and concentrated under reduced pressure. The crude product was purified by silica gel column chromatography eluting with 10% ethyl acetate in heptane. The pure fractions were collected and evaporated to dryness yielding 22 g (76% yield) of intermediate 36 as a colourless oil.

The Following Intermediates were Synthesized by an Analogous Method as Described for Intermediate 36

| Int. No. | Structure | Starting Materials |
|---|---|---|
| Intermediate 37 | 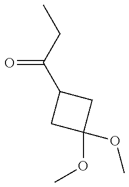 | 3,3- dimethoxycyclobutanecarboxylic acid & ethyl magnesium bromide |
| Intermediate 38 | 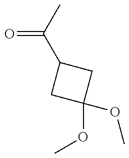 | 3,3- dimethoxycyclobutanecarboxylic acid & methyl magnesium bromide |

### Preparation of Intermediate 39:

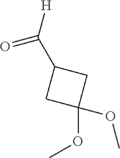

To a solution of N,3,3-trimethoxy-N-methylcyclobutanecarboxamide (1.5 g) in THF (50 mL) was added 1 M lithium aluminum hydride in THF (14 mL, 13.8 mmol) dropwise at −78° C. under nitrogen atmosphere. The resulting mixture was stirred at −78° C. for 3 hours. The mixture was quenched by sodium sulfate decahydrate, and then filtered and concentrated to give intermediate 39 (crude, 1.2 g) as colorless oil, which was used directly in the next step.

### Preparation of Intermediate 40:

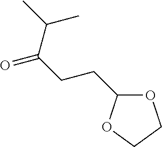

A mixture of magnesium (6.0 g, 247 mmol) and diiodine (100 mg, 0.394 mmol) in THF (100 mL) was stirred at 25° C. Then, 2-(2-bromoethyl)-1,3-dioxolane (20.0 g, 110 mmol) in THF (50 mL) was slowly added to the mixture while maintaining the inner temperature between 20˜30° C. The mixture was stirred at 25° C. for 1 hr and slowly introduced to a solution of N-methoxy-N,2-dimethylpropanamide (10.0 g, 76.24 mmol) in THF (100 mL). The resulting mixture was stirred at 25° C. for 8 hours. The mixture was quenched with 300 mL of saturated solution of ammonium chloride and extracted with ethyl acetate (100 mL×3). The combined organic layers were dried over anhydrous Na₂SO₄, filtered and concentrated under reduced pressure to afford the crude product which was purified by silica gel column chromatography eluting with ethyl acetate in petroleum ether from 0% to 15% to afford intermediate 40 (12.8 g, 67% yield) as colorless oil.

### The Following Intermediate was Synthesized by an Analogous Method as Described for Intermediate 40

| Int. No. | Structure | Starting Materials |
|---|---|---|
| Intermediate 41 | 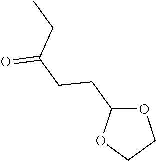 | N-methoxy-N- methylpropionamide & 2-(2- bromoethyl)-1,3-dioxolane |

### Preparation of Intermediate 42, 42a & 42b:

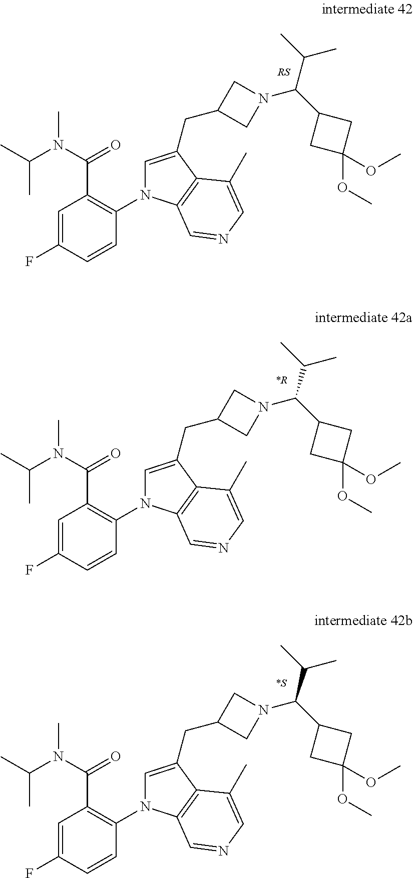

To a solution of intermediate 25 (2.7 g, 6.844 mmol) in methanol (60 mL) was added intermediate 36 (5.0 g, 26.8 mmol), sodium cyanoborohydride (2.149 g, 34.197 mmol) and zinc dichloride (932 mg, 6.837 mmol). The mixture was stirred at 60° C. in a sealed tube for 16 h. After the reaction mixture was cooled down to r.t., the reaction mixture was concentrated and purified by silica gel column chromatography eluting with 10% methanol in dichloromethane to give intermediate 42 (3.8 g) as a white solid, which was separated by chiral Prep. HPLC into the individual enantiomers (separation condition: Column: Chiralpak IA 5 μm 20*250 mm; Mobile Phase: Hex:IPA:DEA=85:15:0.3 at 25 mL/min; Temp: 30° C.; Wavelength: 230 nm) to give the first fraction as intermediate 42a (1.03 g, 26.6% yield) as a white solid and the second fraction as intermediate 42b (1.16 g, 30.0% yield) as a white solid.

The Following Intermediates were Synthesized by an Analogous Method as Described for Intermediate 42

| Int. No. | Structure | Starting Materials |
|---|---|---|
| Intermediate 43 | 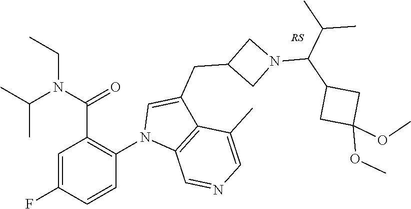 [SMILES: CCN(C(=O)c1cc(F)ccc1-n1cc(CC2CN(C(C(C)C)C3CC(OC)(OC)C3)C2)c2c(C)cncc21)C(C)C] | Intermediate 29 & 36 |
| Intermediate 44 | 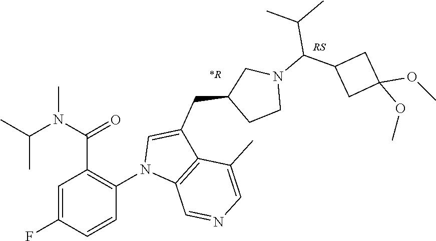 [SMILES: COC1(OC)CC(C(C(C)C)N2CC[C@@H](Cc3cn(-c4ccc(F)cc4C(=O)N(C)C(C)C)c4cncc(C)c34)C2)C1] | Intermediate 26 & 36 |
| Intermediate 45 | 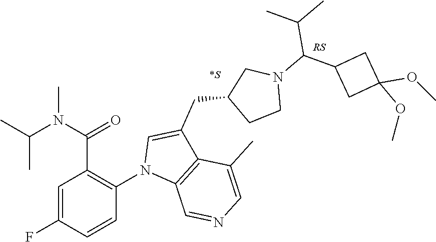 [SMILES: COC1(OC)CC(C(C(C)C)N2CC[C@H](Cc3cn(-c4ccc(F)cc4C(=O)N(C)C(C)C)c4cncc(C)c34)C2)C1] | Intermediate 27 & 36 |
| Intermediate 46 | 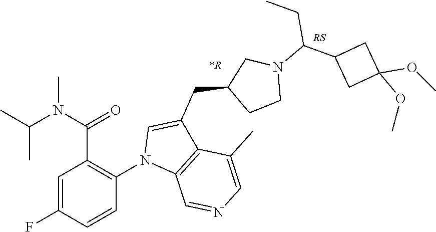 [SMILES: CCC(C1CC(OC)(OC)C1)N1CC[C@@H](Cc2cn(-c3ccc(F)cc3C(=O)N(C)C(C)C)c3cncc(C)c23)C1] | Intermediate 26 & 37 |
| Intermediate 47 | 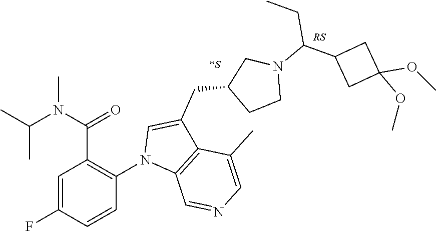 [SMILES: CCC(C1CC(OC)(OC)C1)N1CC[C@H](Cc2cn(-c3ccc(F)cc3C(=O)N(C)C(C)C)c3cncc(C)c23)C1] | Intermediate 27 & 37 |
| Intermediate 48 | 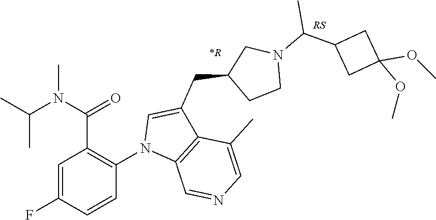 [SMILES: COC1(OC)CC(C(C)N2CC[C@@H](Cc3cn(-c4ccc(F)cc4C(=O)N(C)C(C)C)c4cncc(C)c34)C2)C1] | Intermediate 26 & 38 |
| Intermediate 49 | 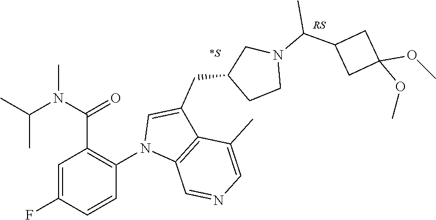 [SMILES: COC1(OC)CC(C(C)N2CC[C@H](Cc3cn(-c4ccc(F)cc4C(=O)N(C)C(C)C)c4cncc(C)c34)C2)C1] | Intermediate 27 & 38 |
| Intermediate 50 | 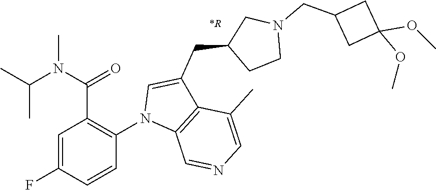 [SMILES: COC1(OC)CC(CN2CC[C@@H](Cc3cn(-c4ccc(F)cc4C(=O)N(C)C(C)C)c4cncc(C)c34)C2)C1] | Intermediate 26 & 39 |
| Intermediate 51 | 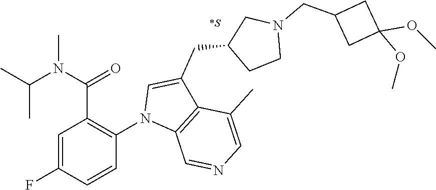 [SMILES: COC1(OC)CC(CN2CC[C@H](Cc3cn(-c4ccc(F)cc4C(=O)N(C)C(C)C)c4cncc(C)c34)C2)C1] | Intermediate 27 & 39 |
| Compound 512 | 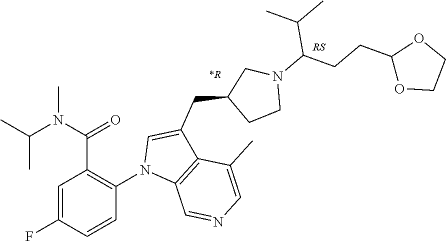 [SMILES: Cc1cncc2c1c(C[C@@H]1CCN(C(CCC3OCCO3)C(C)C)C1)cn2-c1ccc(F)cc1C(=O)N(C)C(C)C] | Intermediate 26 & 40 |
| Compound 513 | 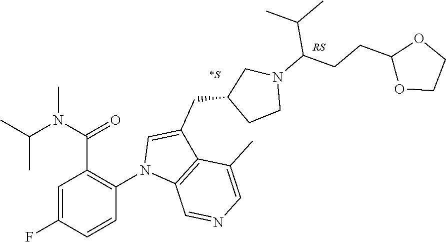 [SMILES: Cc1cncc2c1c(C[C@H]1CCN(C(CCC3OCCO3)C(C)C)C1)cn2-c1ccc(F)cc1C(=O)N(C)C(C)C] | Intermediate 27 & 40 |
| Compound 514 | 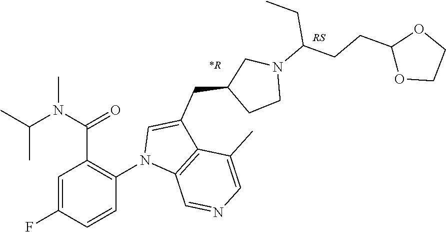 [SMILES: CCC(CCC1OCCO1)N1CC[C@@H](Cc2cn(-c3ccc(F)cc3C(=O)N(C)C(C)C)c3cncc(C)c23)C1] | Intermediate 26 & 41 |
| Compound 515 | 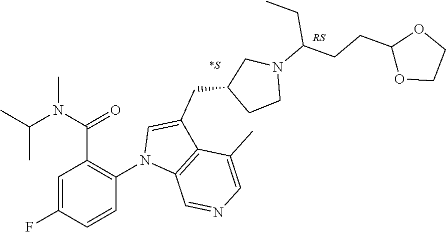 [SMILES: CCC(CCC1OCCO1)N1CC[C@H](Cc2cn(-c3ccc(F)cc3C(=O)N(C)C(C)C)c3cncc(C)c23)C1] | Intermediate 27 & 41 |
| Intermediate 56 | 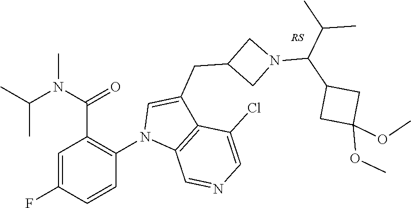 [SMILES: COC1(OC)CC(C(C(C)C)N2CC(Cc3cn(-c4ccc(F)cc4C(=O)N(C)C(C)C)c4cncc(Cl)c34)C2)C1] | Intermediate 30 & 36 |
| Intermediate 57 | 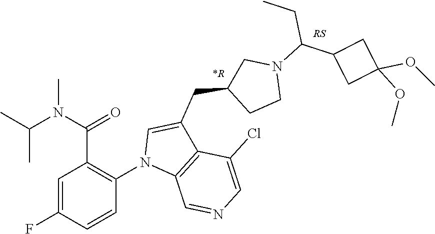 [SMILES: CCC(C1CC(OC)(OC)C1)N1CC[C@@H](Cc2cn(-c3ccc(F)cc3C(=O)N(C)C(C)C)c3cncc(Cl)c23)C1] | Intermediate 31 & 37 |
| Intermediate 58 | 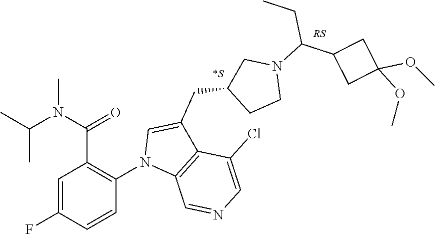 [SMILES: CCC(C1CC(OC)(OC)C1)N1CC[C@H](Cc2cn(-c3ccc(F)cc3C(=O)N(C)C(C)C)c3cncc(Cl)c23)C1] | Intermediate 32 & 37 |
| Compound 370 | 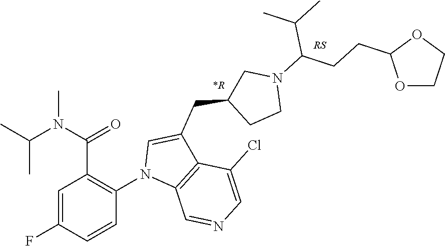 [SMILES: CC(C)C(CCC1OCCO1)N1CC[C@@H](Cc2cn(-c3ccc(F)cc3C(=O)N(C)C(C)C)c3cncc(Cl)c23)C1] | Intermediate 31 & 40 |
| Compound 373 | 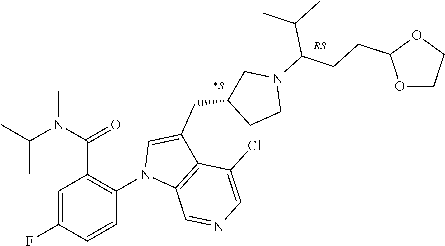 [SMILES: CC(C)C(CCC1OCCO1)N1CC[C@H](Cc2cn(-c3ccc(F)cc3C(=O)N(C)C(C)C)c3cncc(Cl)c23)C1] | Intermediate 32 & 40 |
| Compound 525 | 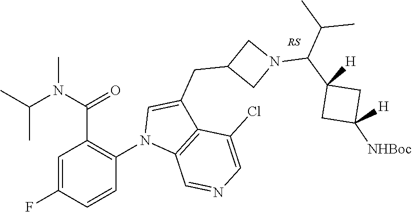 [SMILES: CC(C)C([C@H]1C[C@@H](NC(=O)OC(C)(C)C)C1)N1CC(Cc2cn(-c3ccc(F)cc3C(=O)N(C)C(C)C)c3cncc(Cl)c23)C1] | Intermediate 30 & 34 |

### Preparation of Compound 366 & 367:

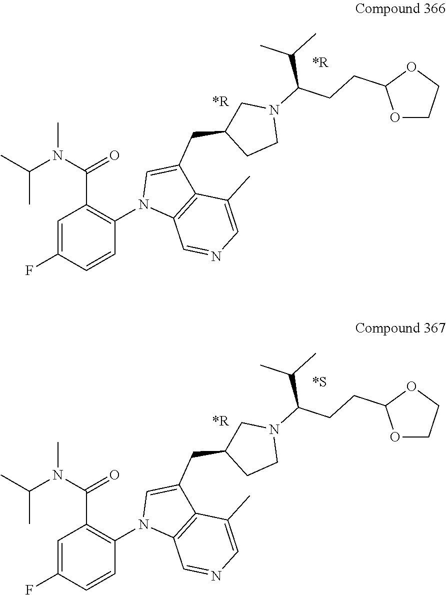 [SMILES: Cc1cncc2c1c(C[C@@H]1CCN([C@H](CCC3OCCO3)C(C)C)C1)cn2-c1ccc(F)cc1C(=O)N(C)C(C)C ; Cc1cncc2c1c(C[C@@H]1CCN([C@H](CCC3OCCO3)C(C)C)C1)cn2-c1ccc(F)cc1C(=O)N(C)C(C)C]

Compound 512 (1.0 g, 1.77 mmol) was separated by SFC (separation condition: Column DAICEL CHIRALCEL OD (250 mm*50 mm, 10 um); Mobile phase: A: 0.1% NH₃H₂O, B: MeOH, A:B=80:20 at 200 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The pure fractions were collected, and the volatiles were removed under vacuum. The first fraction was collected as Compound 366 (220 mg) and the second fraction was collected as Compound 367 (200 mg) as white solid.

### Preparation of Compound 368 & 369:

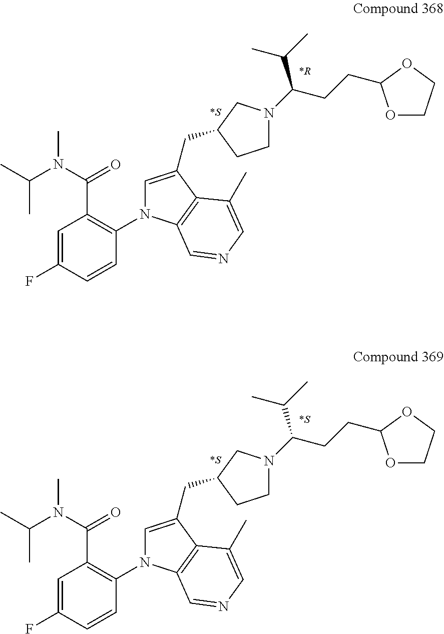 [SMILES: Cc1cncc2c1c(C[C@H]1CCN([C@H](CCC3OCCO3)C(C)C)C1)cn2-c1ccc(F)cc1C(=O)N(C)C(C)C ; Cc1cncc2c1c(C[C@H]1CCN([C@@H](CCC3OCCO3)C(C)C)C1)cn2-c1ccc(F)cc1C(=O)N(C)C(C)C]

Compound 513 (1.0 g, 1.77 mmol) was separated by SFC (separation condition: DAICEL CHIRALPAK AD (250 mm*50 mm, 10 um); Mobile phase: A: 0.1% NH₃H₂O, B: IPA, A:B=75:25 at 200 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The pure fractions were collected, and the volatiles were removed under vacuum. The first fraction was collected as Compound 368 (380 mg, 94.8% purity, 36% yield) and the second fraction was collected as Compound 369 (280 mg, 83.5% purity, 23% yield) as a white solid.

### Preparation of Intermediate 56a & 56b:

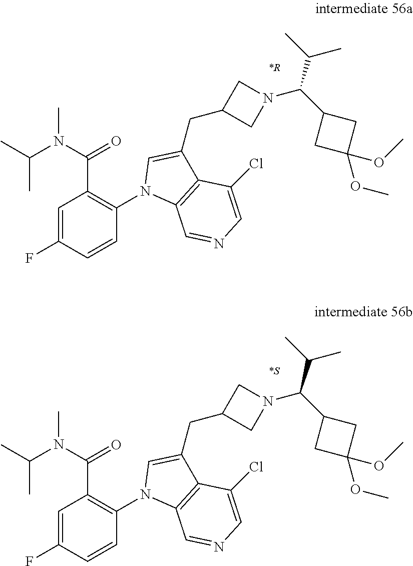

Intermediate 56 (700 mg, 1.20 mmol) was separated by SFC (separation condition: DAICEL CHIRALPAK AD (250 mm*30 mm, 10 um); Mobile phase: A: Supercritical CO₂, B: 0.1% NH₃H₂O IPA, A:B=65:35 at 70 mL/min). The pure fractions were collected and the solvent was evaporated under vacuum to give the products. The first fraction was collected as intermediate 56a (280 mg, 100% purity, 40.0% yield) as a colorless oil and the second fraction was collected as intermediate 56b (300 mg, 99.8% purity, 42.8% yield) as a colorless oil.

### Preparation of Intermediate 58a & 58b:

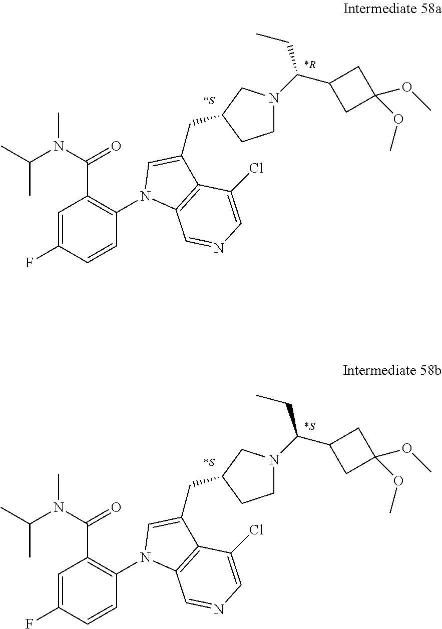

Intermediate 58 (210 mg, 0.36 mmol) was separated by SFC (separation condition: DAICEL CHIRALPAK AD (250 mm*30 mm, 10 um)); Mobile phase: A: Supercritical CO₂, B: 0.1% NH₃H₂O IPA, A:B=75:25 at 60 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The pure fraction was collected, and the solvent was evaporated under vacuum. The first fraction was collected as intermediate 58a (92.0 mg, 98.8% purity, 24.7% yield) as a yellow oil and the second fraction was collected as intermediate 58b (70.0 mg, 98.7% purity, 18.8% yield) as a yellow oil.

### Preparation of Compound 371 & 372:

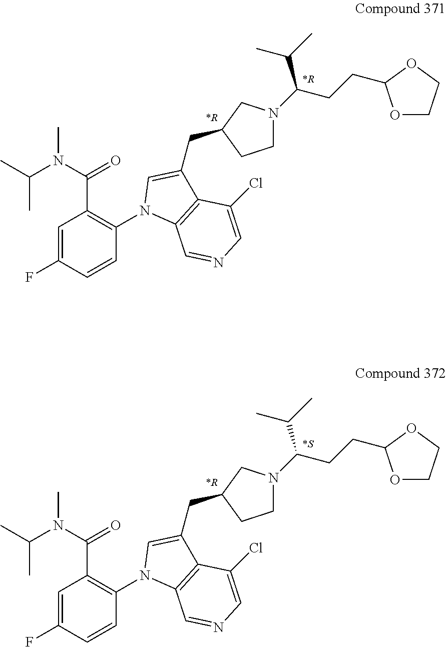 [SMILES: CC(C)[C@@H](CCC1OCCO1)N1CC[C@@H](Cc2cn(-c3ccc(F)cc3C(=O)N(C)C(C)C)c3cncc(Cl)c23)C1 ; CC(C)[C@H](CCC1OCCO1)N1CC[C@@H](Cc2cn(-c3ccc(F)cc3C(=O)N(C)C(C)C)c3cncc(Cl)c23)C1]

Compound 370 (250 mg, 0.427 mmol) was separated by SFC (DAICEL CHIRALCEL OD-H (250 mm*30 mm, 5 um); Mobile phase: A: Supercritical CO₂, B: 0.1% NH₃H₂O MeOH; Isocratic: A:B=75:25; Flow rate: 70 mL/min). The pure fraction was collected, and the solvent was evaporated under vacuum. The first fraction was collected as Compound 371 (80 mg, 92.1% purity, 29% yield) as a yellow oil and the second fraction was collected as Compound 372 (90 mg, 89.6% purity, 32% yield) as a yellow oil.

### Preparation of Compound 374 & 375:

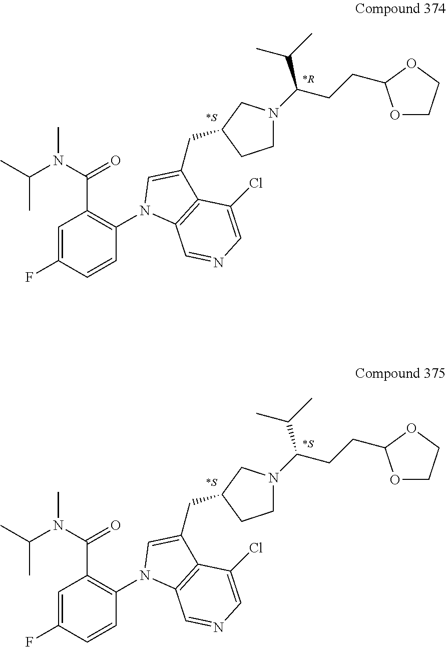 [SMILES: CC(C)[C@@H](CCC1OCCO1)N1CC[C@H](Cc2cn(-c3ccc(F)cc3C(=O)N(C)C(C)C)c3cncc(Cl)c23)C1 ; CC(C)[C@H](CCC1OCCO1)N1CC[C@H](Cc2cn(-c3ccc(F)cc3C(=O)N(C)C(C)C)c3cncc(Cl)c23)C1]

Compound 373 (250 mg, 0.43 mmol) was separated by SFC (DAICEL CHIRALPAK AD (250 mm*30 mm, 10 um); Mobile phase: A: Supercritical CO₂, B: 0.1% NH₃H₂O IPA; Isocratic: A:B=70:30; Flow rate: 70 mL/min). The pure fraction was collected, and the solvent was evaporated under vacuum. The first fraction was collected as Compound 374 (90 mg, 97.0% purity, 35% yield) as a yellow oil and the second fraction was collected as Compound 75 (80 mg, 97.7% purity, 31% yield) as yellow oil as a yellow oil.

### Preparation of Intermediate 67a:

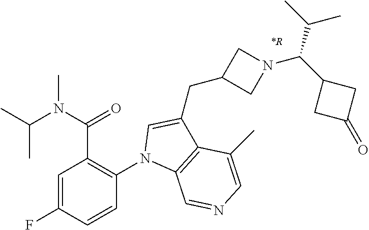 [SMILES: Cc1cncc2c1c(CC1CN([C@H](C(C)C)C3CC(=O)C3)C1)cn2-c1ccc(F)cc1C(=O)N(C)C(C)C]

To a solution of intermediate 42a (110 mg, 95% purity from LCMS, 0.185 mmol) in acetonitrile (2.5 mL) was added aqueous hydrochloric acid solution (1 N, 0.8 mL) at room temperature. The reaction mixture was heated up to 35° C. and stirred at this temperature for 40 minutes. After the reaction mixture was cooled down to r.t., the reaction mixture was basified with saturated NaHCO₃ aqueous solution until the pH=−8 and extracted with DCM (20 mL) twice. The combined organic layers were washed with brine (20 mL), dried over sodium sulfate, filtered and concentrated to afford intermediate 62, which was used in the next step without further purification.

The Following Intermediates were Synthesized by an Analogous Method as Described for Intermediate 62a

| Int. No. | Structure | Starting Materials |
|---|---|---|
| Intermediate 62b | 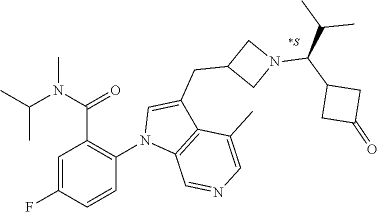 [SMILES: Cc1cncc2c1c(CC1CN([C@@H](C(C)C)C3CC(=O)C3)C1)cn2-c1ccc(F)cc1C(=O)N(C)C(C)C] | Intermediate 42b |
| Intermediate 63 | 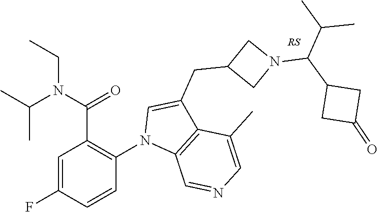 [SMILES: CCN(C(=O)c1cc(F)ccc1-n1cc(CC2CN(C(C(C)C)C3CC(=O)C3)C2)c2c(C)cncc21)C(C)C] | Intermediate 43 |
| Intermediate 64 | 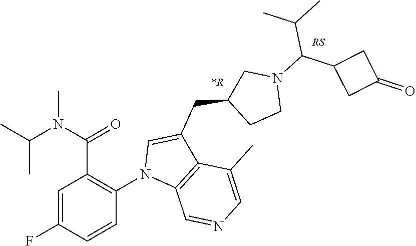 [SMILES: Cc1cncc2c1c(C[C@@H]1CCN(C(C(C)C)C3CC(=O)C3)C1)cn2-c1ccc(F)cc1C(=O)N(C)C(C)C] | Intermediate 44 |
| Intermediate 65 | 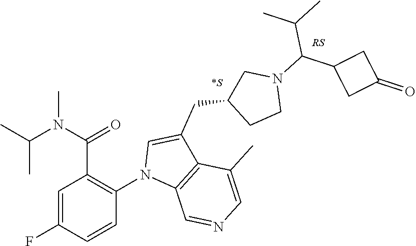 [SMILES: Cc1cncc2c1c(C[C@H]1CCN(C(C(C)C)C3CC(=O)C3)C1)cn2-c1ccc(F)cc1C(=O)N(C)C(C)C] | Intermediate 45 |
| Intermediate 66 | 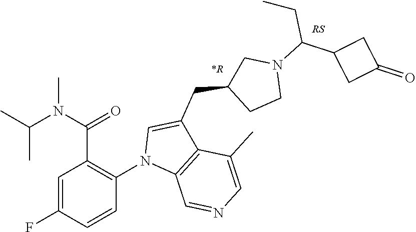 [SMILES: CCC(C1CC(=O)C1)N1CC[C@@H](Cc2cn(-c3ccc(F)cc3C(=O)N(C)C(C)C)c3cncc(C)c23)C1] | Intermediate 46 |
| Intermediate 67 | 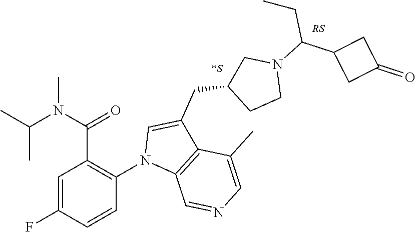 [SMILES: CCC(C1CC(=O)C1)N1CC[C@H](Cc2cn(-c3ccc(F)cc3C(=O)N(C)C(C)C)c3cncc(C)c23)C1] | Intermediate 47 |
| Intermediate 68 | 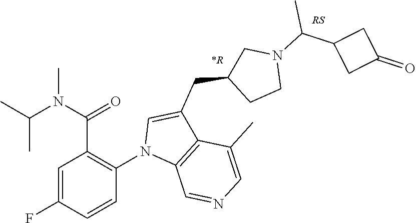 [SMILES: Cc1cncc2c1c(C[C@@H]1CCN(C(C)C3CC(=O)C3)C1)cn2-c1ccc(F)cc1C(=O)N(C)C(C)C] | Intermediate 48 |
| Intermediate 69 | 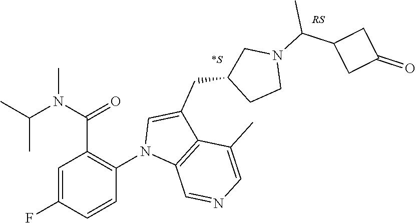 [SMILES: Cc1cncc2c1c(C[C@H]1CCN(C(C)C3CC(=O)C3)C1)cn2-c1ccc(F)cc1C(=O)N(C)C(C)C] | Intermediate 49 |
| Intermediate 70 | 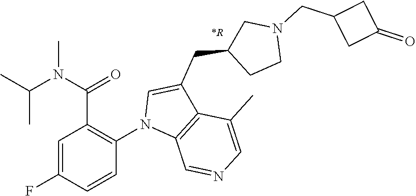 [SMILES: Cc1cncc2c1c(C[C@@H]1CCN(CC3CC(=O)C3)C1)cn2-c1ccc(F)cc1C(=O)N(C)C(C)C] | Intermediate 50 |
| Intermediate 71 | 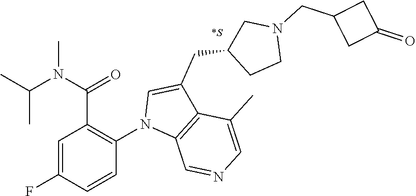 [SMILES: Cc1cncc2c1c(C[C@H]1CCN(CC3CC(=O)C3)C1)cn2-c1ccc(F)cc1C(=O)N(C)C(C)C] | Intermediate 51 |
| Intermediate 72 | 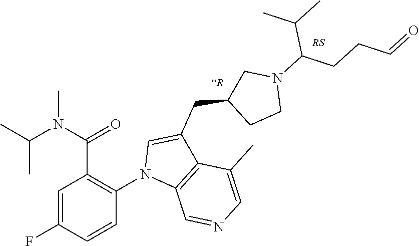 [SMILES: Cc1cncc2c1c(C[C@@H]1CCN(C(CCC=O)C(C)C)C1)cn2-c1ccc(F)cc1C(=O)N(C)C(C)C] | Compound 512 |
| Intermediate 72a | 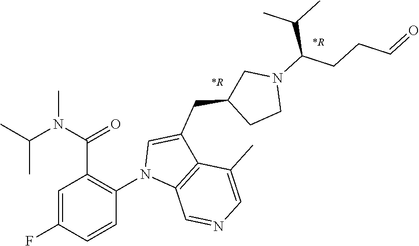 [SMILES: Cc1cncc2c1c(C[C@@H]1CCN([C@H](CCC=O)C(C)C)C1)cn2-c1ccc(F)cc1C(=O)N(C)C(C)C] | Compound 366 |
| Intermediate 72b | 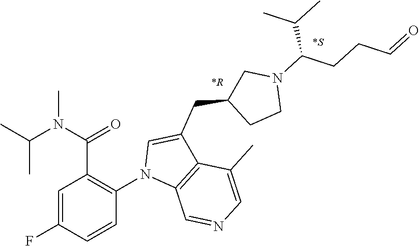 [SMILES: Cc1cncc2c1c(C[C@@H]1CCN([C@@H](CCC=O)C(C)C)C1)cn2-c1ccc(F)cc1C(=O)N(C)C(C)C] | Compound 367 |
| Intermediate 73 | 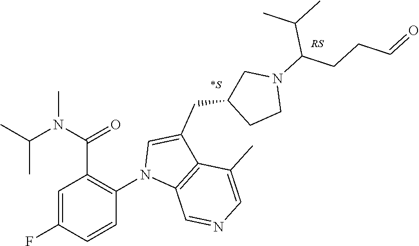 [SMILES: Cc1cncc2c1c(C[C@H]1CCN(C(CCC=O)C(C)C)C1)cn2-c1ccc(F)cc1C(=O)N(C)C(C)C] | Compound 513 |
| Intermediate 73a | 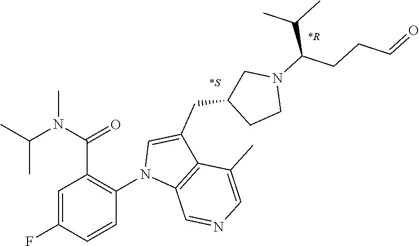 [SMILES: Cc1cncc2c1c(C[C@H]1CCN([C@H](CCC=O)C(C)C)C1)cn2-c1ccc(F)cc1C(=O)N(C)C(C)C] | Compound 368 |
| Intermediate 73b | 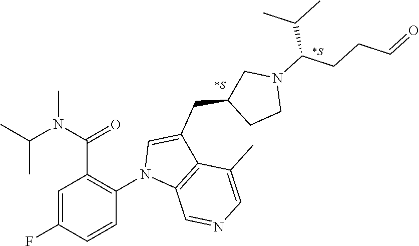 [SMILES: Cc1cncc2c1c(C[C@@H]1CCN([C@@H](CCC=O)C(C)C)C1)cn2-c1ccc(F)cc1C(=O)N(C)C(C)C] | Compound 369 |
| Intermediate 74 | 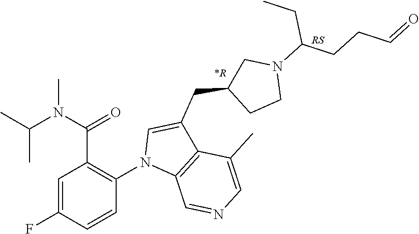 [SMILES: CCC(CCC=O)N1CC[C@@H](Cc2cn(-c3ccc(F)cc3C(=O)N(C)C(C)C)c3cncc(C)c23)C1] | Compound 514 |
| Intermediate 75 | 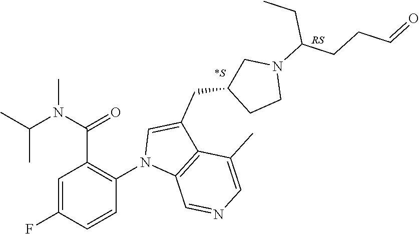 [SMILES: CCC(CCC=O)N1CC[C@H](Cc2cn(-c3ccc(F)cc3C(=O)N(C)C(C)C)c3cncc(C)c23)C1] | Compound 515 |
| Intermediate 76a | 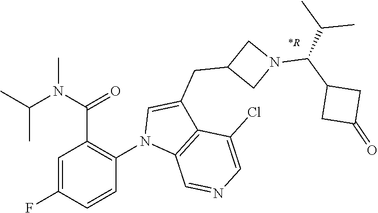 [SMILES: CC(C)[C@H](C1CC(=O)C1)N1CC(Cc2cn(-c3ccc(F)cc3C(=O)N(C)C(C)C)c3cncc(Cl)c23)C1] | Intermediate 56a |
| Intermediate 76b | 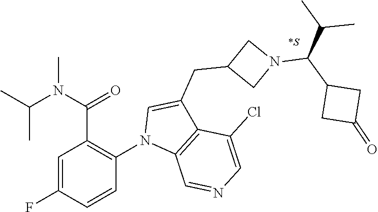 [SMILES: CC(C)[C@@H](C1CC(=O)C1)N1CC(Cc2cn(-c3ccc(F)cc3C(=O)N(C)C(C)C)c3cncc(Cl)c23)C1] | Intermediate 56b |
| Intermediate 77 | 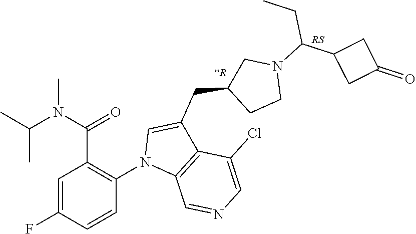 [SMILES: CCC(C1CC(=O)C1)N1CC[C@@H](Cc2cn(-c3ccc(F)cc3C(=O)N(C)C(C)C)c3cncc(Cl)c23)C1] | Intermediate 57 |
| Intermediate 78a | 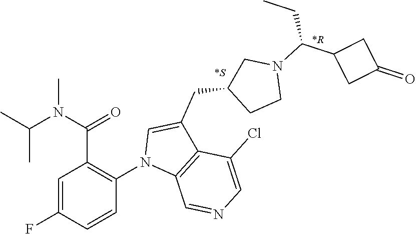 [SMILES: CC[C@H](C1CC(=O)C1)N1CC[C@H](Cc2cn(-c3ccc(F)cc3C(=O)N(C)C(C)C)c3cncc(Cl)c23)C1] | Intermediate 58a |
| Intermediate 78b | 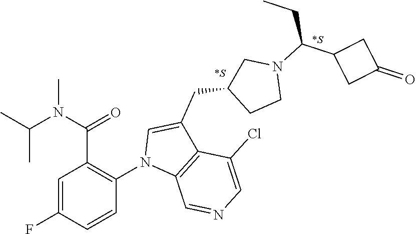 [SMILES: CC[C@@H](C1CC(=O)C1)N1CC[C@H](Cc2cn(-c3ccc(F)cc3C(=O)N(C)C(C)C)c3cncc(Cl)c23)C1] | Intermediate 58b |
| Intermediate 79a | 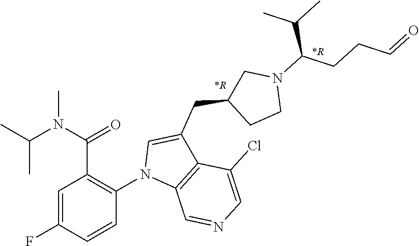 [SMILES: CC(C)[C@@H](CCC=O)N1CC[C@@H](Cc2cn(-c3ccc(F)cc3C(=O)N(C)C(C)C)c3cncc(Cl)c23)C1] | Compound 371 |
| Intermediate 79b | 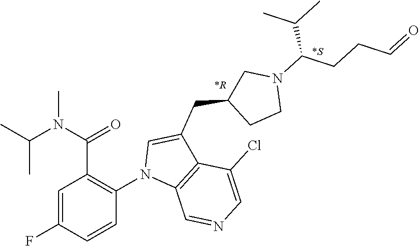 [SMILES: CC(C)[C@H](CCC=O)N1CC[C@@H](Cc2cn(-c3ccc(F)cc3C(=O)N(C)C(C)C)c3cncc(Cl)c23)C1] | Compound 372 |
| Intermediate 80a | 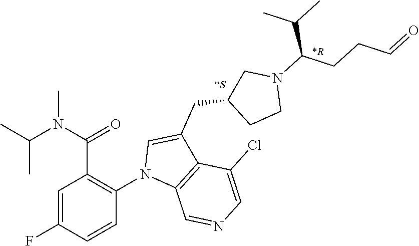 [SMILES: CC(C)[C@@H](CCC=O)N1CC[C@H](Cc2cn(-c3ccc(F)cc3C(=O)N(C)C(C)C)c3cncc(Cl)c23)C1] | Compound 374 |
| Intermediate 80b | 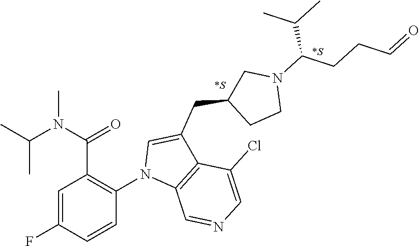 [SMILES: CC(C)[C@H](CCC=O)N1CC[C@@H](Cc2cn(-c3ccc(F)cc3C(=O)N(C)C(C)C)c3cncc(Cl)c23)C1] | Compound 375 |

### Preparation of Compound 376:

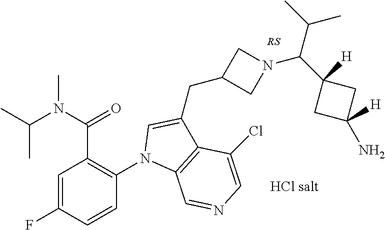 [SMILES: CC(C)C([C@H]1C[C@@H](N)C1)N1CC(Cc2cn(-c3ccc(F)cc3C(=O)N(C)C(C)C)c3cncc(Cl)c23)C1]

A 4M solution of HCl in dioxane (1.90 mL, 7.60 mmol) was added to a solution of compound 525 (310 mg, 0.48 mmol) in dioxane (3 mL) at 0° C. After stirring at r.t. for 1 hr, the reaction mixture was concentrated under reduced pressure to give Compound 376 (420 mg, crude), which was used in next step without further purification.

### Preparation of Intermediate 82:

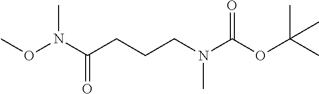 [SMILES: CON(C)C(=O)CCCN(C)C(=O)OC(C)(C)C]

EDCI·HCl (35.0 g, 183 mmol) was added to a solution of 4-((tert-butoxycarbonyl) (methyl)amino)butanoic acid (28.0 g, 129 mmol), N,O-dimethylhydroxylamine hydrochloride (16.0 g, 164 mmol), HOBt (17.5 g, 130 mmol) and 4-methylmorpholine (78.0 g, 771 mmol) in CHCl₃ (500 mL). After stirring at r.t. for 16 hours, the reaction mixture was subsequently washed with water (250 mL×2), 0.1N aq. HCl solution (250 mL×2), sat. aq. NaHCO₃ solution (250 mL×2) and brine (250 mL×2). The organic layer was dried over anhydrous Na₂SO₄, filtered, and concentrated in vacuo to give the crude product which was purified by silica gel column chromatography eluting with ethyl acetate in petroleum ether from 0% to 50% to afford intermediate 82 (27 g, 80% yield) as a colorless oil.

### Preparation of Intermediate 83:

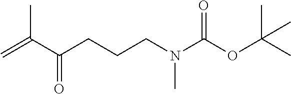 [SMILES: C=C(C)C(=O)CCCN(C)C(=O)OC(C)(C)C]

At 0° C., to a solution of intermediate 82 (27.0 g, 104 mmol) in THF (800 mL) was added prop-1-en-2-ylmagnesium bromide (260 mL, 260 mmol, 1 M) under N₂. The mixture was stirred at 0° C. under N₂ for 1 hour, slowly warmed up to room temperature and stirred at room temperature for 16 hours. The mixture was quenched with sat. aq. NH₄Cl solution (400 mL) and extracted with EtOAc (500 mL×3). The combined organic layers were washed with H₂O (300 mL×2) and brine (300 mL), dried over Na₂SO₄, filtered and concentrated under reduced pressure to give intermediate 83 (25 g, crude) as light yellow oil, which was used in the next step without further purification.

### _Preparation of Intermediate 84:

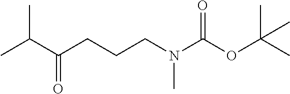 [SMILES: CC(C)C(=O)CCCN(C)C(=O)OC(C)(C)C]

To a solution of intermediate 83 (11.0 g, crude) in MeOH (100 mL) was added 10 w/w % Pd/C (1 g) under N₂ atmosphere. The mixture was degassed under vacuum and purged with H₂ three times. The mixture was stirred at rt for 2 hours under H₂ (15 psi) atmosphere. The reaction mixture was filtered through a pad of Celite®, and the filter cake was washed with MeOH (30×2 mL). The filtrate was concentrated under reduced pressure to give the crude product which was purified by silica gel column chromatography eluting with 20% ethyl acetate in petroleum ether to give intermediate 84 (9.5 g, 86% yield) as a colorless oil.

### Preparation of Intermediate 85:

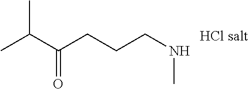

To a solution of intermediate 84 (20.0 g, 82.2 mmol) in DCM (200 mL) was added 4M HCl in dioxane solution (120 mL, 480 mmol). After stirring at r.t for 1 hour, the reaction mixture was concentrated under reduce pressure to give intermediate 85 (17.8 g, crude) as a white solid, which was used in next step without further purification.

### Preparation of Intermediate 86:

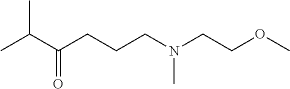

To a solution of intermediate 85 (70 g, crude), K₂CO₃ (224 g, 1621 mmol) and NaI (146 g, 974 mmol) in DMF (700 ml) was added 1-bromo-2-methoxyethane (54 g, 389 mmol). The mixture was stirred at 50° C. for 5 hours. The insoluble residues were removed via filtration, and the filtrate was concentrated under reduced pressure to give the crude product, which was poured into water (500 mL) and extracted with ethyl acetate (500 mL×3). The combined organic layers were washed with brine (100 mL×3), 5% aq. LiCl solution (100 mL×3) and water (100 mL), dried over Na₂SO₄, filtered, and concentrated under reduced pressure to give intermediate 86 (25 g, 38% yield) as a brown oil.

### Preparation of Intermediate 87:

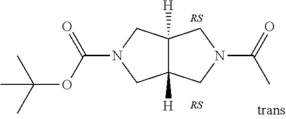 [SMILES: CC(=O)N1C[C@@H]2CN(C(=O)OC(C)(C)C)C[C@H]2C1]

To a solution of tert-butyl (trans)-rel-octahydropyrrolo[3,4-c]pyrrole-2-carboxylate hemioxalate (1.00 g, 3.89 mmol) in anhydrous dichloromethane (20.0 mL) was added triethylamine (2.00 g, 19.8 mmol). Then acetic anhydride (600 mg, 5.88 mmol) was added dropwise, and the reaction mixture was stirred at 25° C. for 70 minutes. The reaction mixture was diluted with dichloromethane (30 mL) and washed with water (20 mL×1), brine (20 mL×1) and saturated aqueous sodium bicarbonate solution (20 mL×1). The organic phase was dried over Na₂SO₄, filtered, and concentrated under reduced pressure to give intermediate 87 (990 mg, 95.0% purity, 95.2% yield) as a white solid.

### Preparation of Intermediate 88:

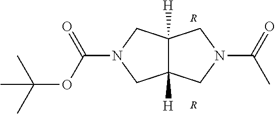

Intermediate 87 was separated by SFC (separation condition: DAICEL CHIRALPAK AS (250 mm*50 mm, 10 um)); Mobile phase: A: Supercritical CO₂, B: 0.1% NH₃H₂O EtOH, A:B=85:15 at 200 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The second fraction was collected as intermediate 88 (3.36 g, 97.0% purity, 43.2% yield) as a white solid.

### Preparation of Intermediate 89:

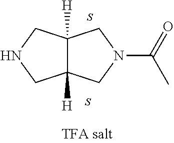

To a solution of intermediate 88 (300 mg, 1.18 mmol) in anhydrous dichloromethane (2 mL) was added trifluoroacetic acid (2 mL). After stirring at 25° C. for 1 h, the reaction mixture was concentrated under reduced pressure to give intermediate 89 (300 mg, crude) as yellow oil, which was used in the next step without further purification.

### Preparation of Compound 377:

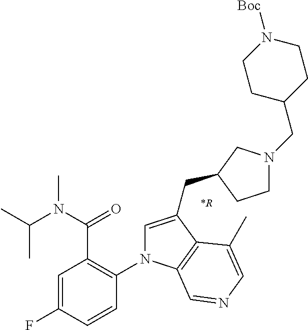 [SMILES: Cc1cncc2c1c(C[C@@H]1CCN(CC3CCN(C(=O)OC(C)(C)C)CC3)C1)cn2-c1ccc(F)cc1C(=O)N(C)C(C)C]

To a solution of intermediate 26 (100 mg, 95% purity, 0.223 mmol) in methanol (3 mL) were added tert-butyl 4-formylpiperidine-1-carboxylate (104 mg, 0.465 mmol) and sodium triacetoxyborohydride (98.1 mg, 0.465 mmol). After stirring at r.t. for 6 hours, the reaction mixture was concentrated, and the residue was purified by preparative TLC (10% MeOH in DCM) to give Compound 377 (130 mg, 95% purity, 87.7% yield) as a white oil.

### Preparation of Compound 378:

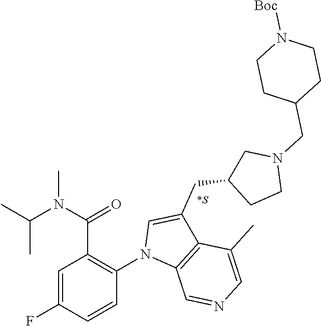 [SMILES: Cc1cncc2c1c(C[C@H]1CCN(CC3CCN(C(=O)OC(C)(C)C)CC3)C1)cn2-c1ccc(F)cc1C(=O)N(C)C(C)C]

To a solution of intermediate 27 (3.5 g, 95%, 8.14 mmol) in DCM (80 mL) was added tert-butyl 4-formylpiperidine-1-carboxylate (3.66 g, 16.3 mmol) and sodium triacetoxyborohydride (2.58 g, 12.2 mmol). After stirring at room temperature for 6 hours, the reaction mixture was poured into saturated aqueous sodium bicarbonate solution and extracted with dichloromethane (80 mL) twice. The combined organic layers were washed with brine (80 mL), dried over Na₂SO₄, filtered and concentrated under reduced pressure to afford the crude product, which was purified by silica gel column chromatography eluting with methanol in dichloromethane from 0% to 6% to give Compound 378 (4.66 g, 95% purity, 89.8% yield) as a white oil.

The Following Intermediates and Compounds were Synthesized by an Analogous Method as Described for Compound 378

In case reactions were performed with a ketone starting material, a typical procedure makes use of either 2 eq. acetic acid or 2 eq. of zinc(II)chloride (ZnC₂), in the presence of 2 eq. sodium cyanoborohydride (NaCNBH₃), in methanol at 50° C. or 70° C. overnight.

| Int./Co No. | Structure | Starting Materials |
|---|---|---|
| Compound 62 | 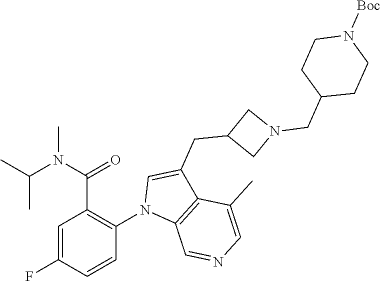 [SMILES: Cc1cncc2c1c(CC1CN(CC3CCN(C(=O)OC(C)(C)C)CC3)C1)cn2-c1ccc(F)cc1C(=O)N(C)C(C)C] | Intermediate 25 & tert-butyl 4- formylpiperidine-1-carboxylate |
| Compound 382 | 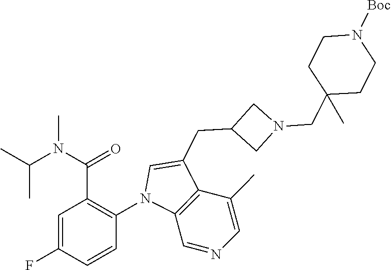 [SMILES: Cc1cncc2c1c(CC1CN(CC3(C)CCN(C(=O)OC(C)(C)C)CC3)C1)cn2-c1ccc(F)cc1C(=O)N(C)C(C)C] | Intermediate 25 & tert-butyl 4- formyl-4-methylpiperidine-1- carboxylate |
| Compound 383 | 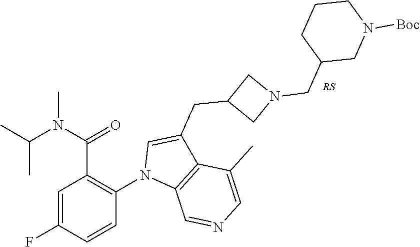 [SMILES: Cc1cncc2c1c(CC1CN(CC3CCCN(C(=O)OC(C)(C)C)C3)C1)cn2-c1ccc(F)cc1C(=O)N(C)C(C)C] | Intermediate 25 & tert-butyl 3- formylpiperidine-1-carboxylate |
| Compound 384 | 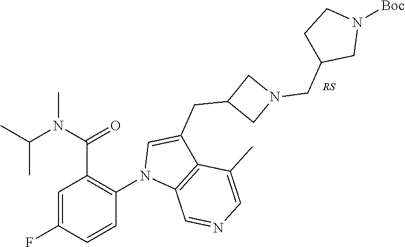 [SMILES: Cc1cncc2c1c(CC1CN(CC3CCN(C(=O)OC(C)(C)C)C3)C1)cn2-c1ccc(F)cc1C(=O)N(C)C(C)C] | Intermediate 25 & tert-butyl 3- formylpyrrolidine-1-carboxylate |
| Compound 380 | 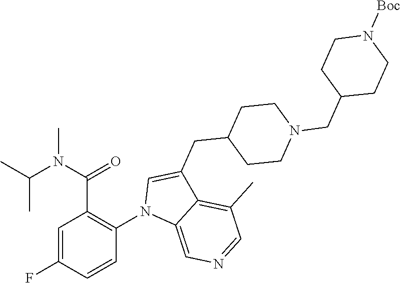 [SMILES: Cc1cncc2c1c(CC1CCN(CC3CCN(C(=O)OC(C)(C)C)CC3)CC1)cn2-c1ccc(F)cc1C(=O)N(C)C(C)C] | Intermediate 28 & tert-butyl 4- formylpiperidine-1-carboxylate |
| Compound 385 | 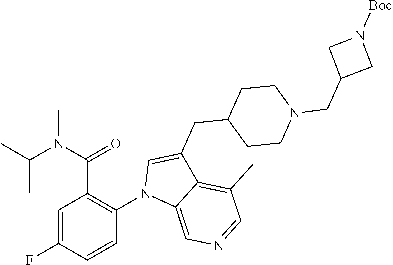 [SMILES: Cc1cncc2c1c(CC1CCN(CC3CN(C(=O)OC(C)(C)C)C3)CC1)cn2-c1ccc(F)cc1C(=O)N(C)C(C)C] | Intermediate 28 & tert-butyl 3- formylazetidine-1-carboxylate |
| Intermediate 157 | 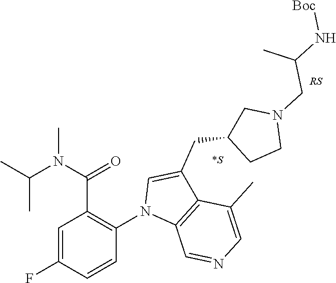 | Intermediate 27 & tert-butyl (1- oxopropan-2-yl)carbamate |
| Compound 386 | 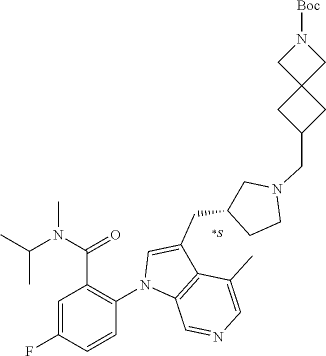 | Intermediate 27 & tert-butyl 6- formyl-2-azaspiro[3.3]heptane- 2-carboxylate |
| Compound 387 & Compound 388 | 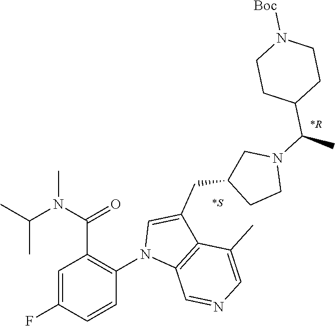 | Intermediate 27 & tert-butyl 4- acetylpiperidine-1-carboxylate The product was separated by supercritical fluid chromatography (separation condition: DAICEL CHIRALPAK AD(250 mm*30 mm, 10 um)); Mobile phase: A: Supercritical CO₂, B: 0.1% NH₃H₂O IPA, A:B = 75:25 at 70 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 387 and the second fraction as Compound 388 |
| | 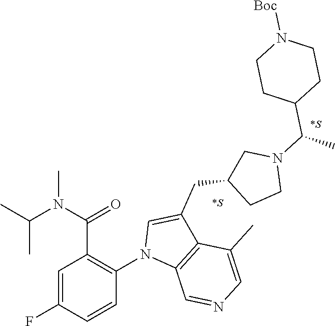 | |
| Intermediate 171 | 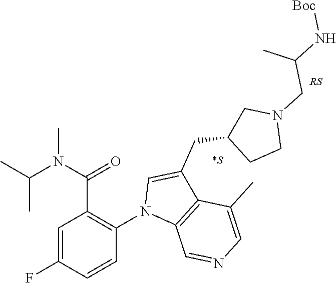 | Intermediate 27 & tert-butyl (1- oxopropan-2-yl)carbamate |
| Compound 389 | 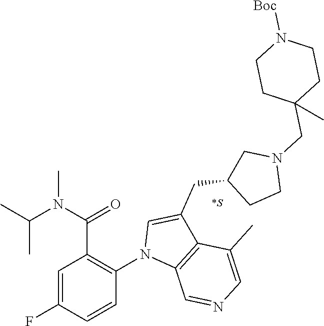 | Intermediate 27 & tert-butyl 4- formyl-4-methylpiperidine-1- carboxylate |
| Compound 501 | 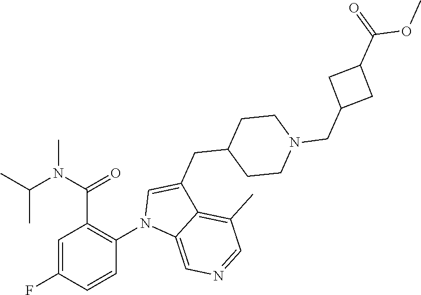 [SMILES: COC(=O)C1CC(CN2CCC(Cc3cn(-c4ccc(F)cc4C(=O)N(C)C(C)C)c4cncc(C)c34)CC2)C1] | Intermediate 28 & 177 |
| Compound 391 | 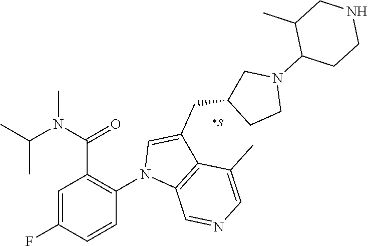 | Intermediate 27 & 3- methylpiperidin-4-one |
| Compound 392 | 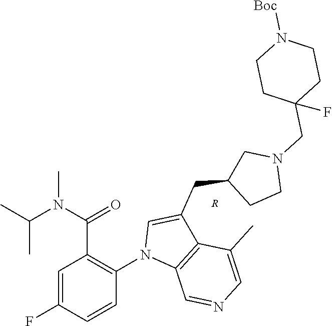 | Intermediate 202 & tert-butyl 4- fluoro-4-formylpiperidine-1- carboxylate |
| Compound 394 | 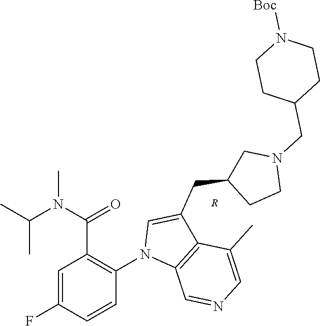 | Intermediate 202 & tert-butyl 4- formylpiperidine-1-carboxylate |
| Compound 395 & Compound 396 | 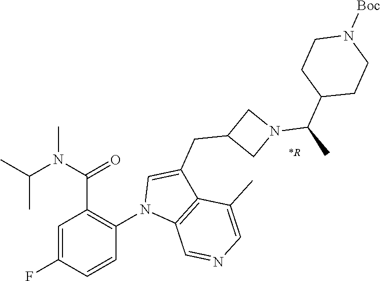 [SMILES: Cc1cncc2c1c(CC1CN([C@H](C)C3CCN(C(=O)OC(C)(C)C)CC3)C1)cn2-c1ccc(F)cc1C(=O)N(C)C(C)C] | Intermediate 25 & tert-butyl 4- acetylpiperidine-1-carboxylate The product was separated by SFC (separation condition: DAICEL CHIRALPAK AD(250 mm*30 mm, 10 um); Mobile phase: A: 0.1% NH₃H₂O, B: EtOH, A:B = 75:25 at 70 mL/min; Column Temp: 38; Nozzle Pressure: 100Bar; Nozzle Temp: 60; Evaporator Temp: 20; Trimmer Temp: 25; Wavelength: 220 nm). The first fraction was collected as Compound 395 & the second fraction as Compound 396. |
| | 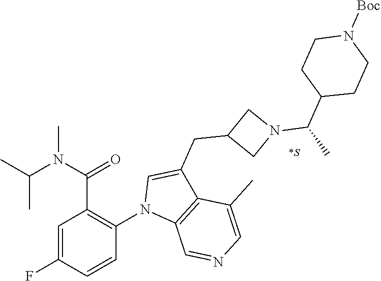 [SMILES: Cc1cncc2c1c(CC1CN([C@@H](C)C3CCN(C(=O)OC(C)(C)C)CC3)C1)cn2-c1ccc(F)cc1C(=O)N(C)C(C)C] | |
| Compound 397 | 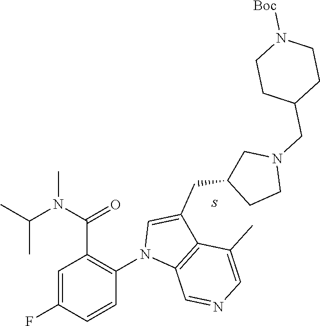 | Intermediate 225 & tert-butyl 4- formylpiperidine-1-carboxylate |
| Compound 526a & Compound 526b | 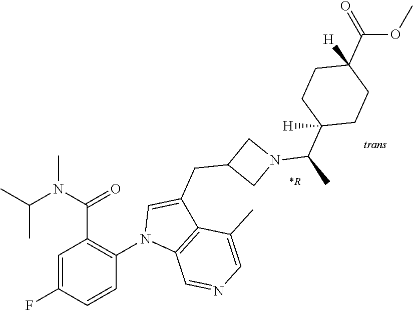 [SMILES: COC(=O)[C@H]1CC[C@H]([C@@H](C)N2CC(Cc3cn(-c4ccc(F)cc4C(=O)N(C)C(C)C)c4cncc(C)c34)C2)CC1] | Intermediate 25 & (trans)- methyl 4- acetylcyclohexanecarboxylate. The product was separated by SFC (separation condition: DAICEL CHIRALPAK IC(250 mm*30 mm, 10 um)); Mobile phase: A: Supercritical CO₂, B: 0.1% NH₃H₂O MeOH, A:B = 50:50 at 80 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 526a & the second fraction as Compound 526b. |
| | 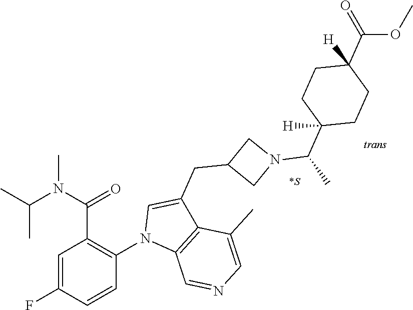 [SMILES: COC(=O)[C@H]1CC[C@H]([C@H](C)N2CC(Cc3cn(-c4ccc(F)cc4C(=O)N(C)C(C)C)c4cncc(C)c34)C2)CC1] | |
| Compound 398 & Compound 399 | 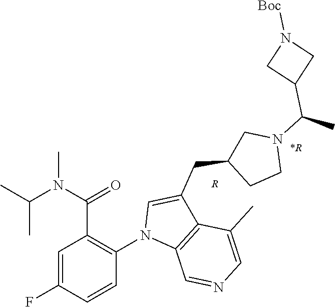 | Intermediate 202 & tert-butyl 3- acetylazetidine-1-carboxylate The product was separated by SFC (separation condition: DAICEL CHIRALPAK IC(250 mm*30 mm, 10 um)); Mobile phase: A: Supercritical CO₂, B: 0.1% NH₃H₂O MeOH, A:B = 50:50 at 80 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 398 & the second fraction as Compound 399. |
| | 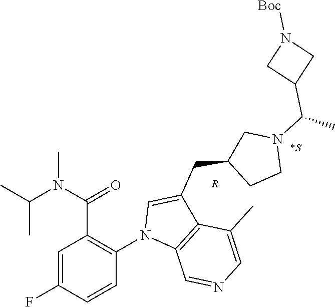 | |
| Compound 400 | 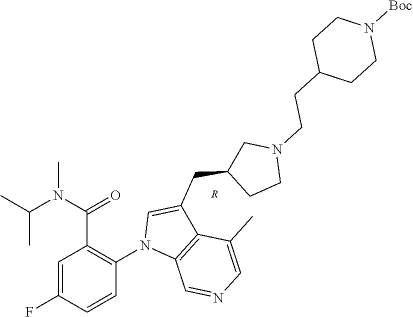 | Intermediate 202 & tert-butyl 4- (2-oxoethyl)piperidine-1- carboxylate |
| Compound 401 | 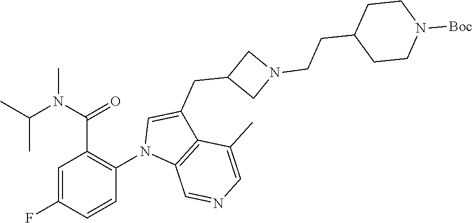 [SMILES: Cc1cncc2c1c(CC1CN(CCC3CCN(C(=O)OC(C)(C)C)CC3)C1)cn2-c1ccc(F)cc1C(=O)N(C)C(C)C] | Intermediate 25 & tert-butyl 4- (2-oxoethyl)piperidine-1- carboxylate |
| Compound 402 & Compound 403 | 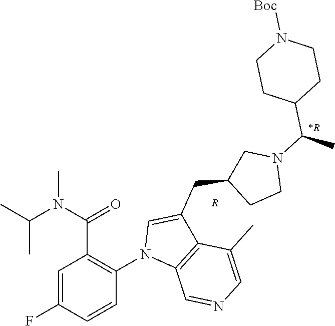 | Intermediate 202 & tert-butyl 4- acetylpiperidine-1-carboxylate The product was separated by SFC (separation condition: DAICEL CHIRALPAK AD(250 mm*50 mm, 10 um); Mobile phase: A: Supercritical CO₂, B: 0.1% NH₃H₂O IPA, A:B = 65:35 at 200 mL/min; Column Temp: 38; Nozzle Pressure: 100Bar; Nozzle Temp: 60; Evaporator Temp: 20; Trimmer Temp: 25; Wavelength: 220 nm). The first fraction was collected as Compound 402 & the second as Compound 403. |
| | 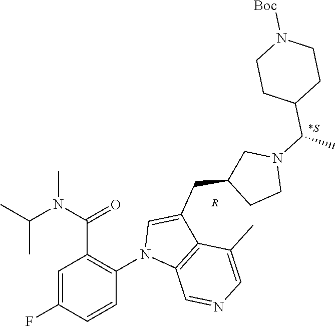 | |
| Compound 404 & Compound 405 | 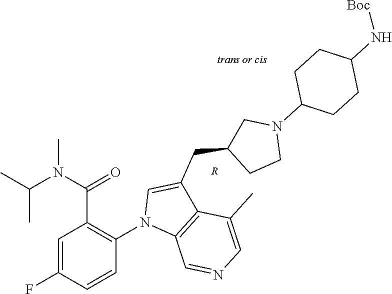 | Intermediate 202 & tert-butyl (4- oxocyclohexyl)carbamate |
| | 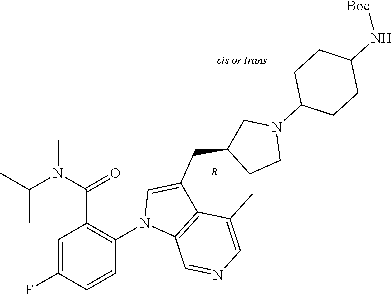 | |
| Compound 407 | 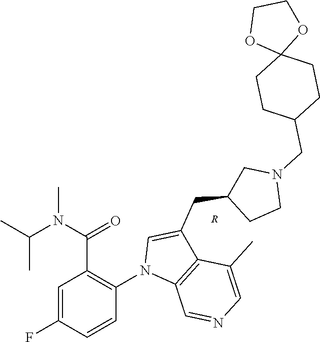 | Intermediate 202 & 1,4- dioxaspiro[4.5]decane-8- carbaldehyde |
| Compound 408 | 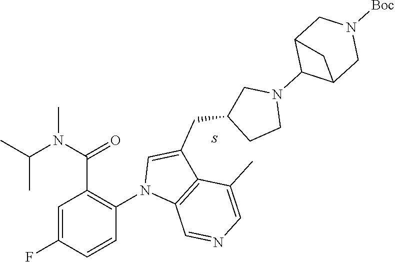 | Intermediate 225 & 3-Boc-6- oxo-3-aza-bicyclo[3.1.1]heptane |
| Compound 409 | 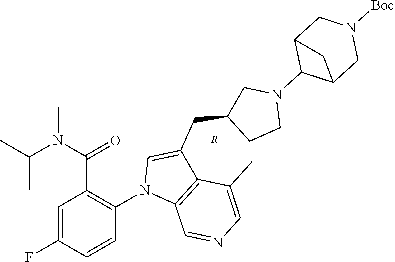 | Intermediate 202 & 3-Boc-6- oxo-3-aza-bicyclo[3.1.1]heptane |
| Compound 410 & Compound 411 | 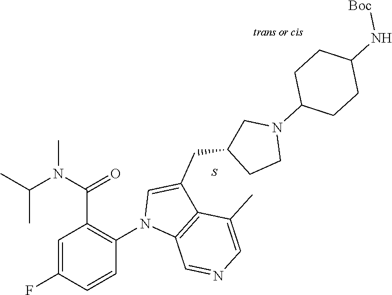 | Intermediate 225 & tert-butyl (4- oxocyclohexyl)carbamate |
| | 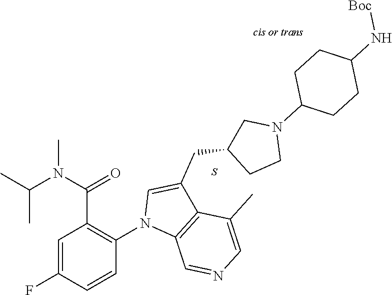 | |
| Compound 412 | 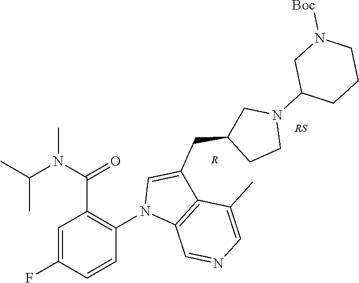 | Intermediate 202 & tert-butyl 3- oxopiperidine-1-carboxylate |
| Compound 413 | 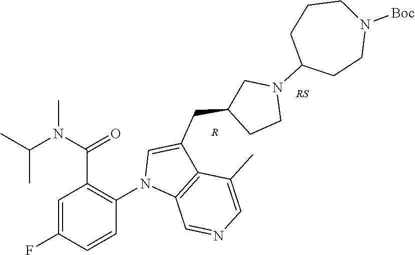 | Intermediate 202 & tert-butyl 4- oxoazepane-1-carboxylate |
| Compound 414 | 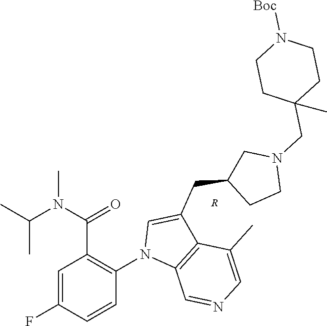 | Intermediate 202 & tert-butyl 4- formyl-4-methylpiperidine-1- carboxylate |
| Compound 415 | 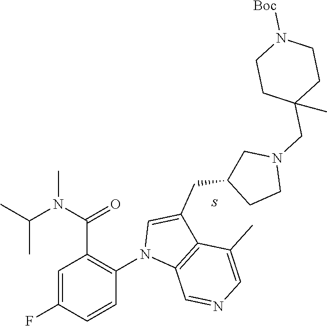 | Intermediate 225 & tert-butyl 4- formyl-4-methylpiperidine-1- carboxylate |
| Compound 416 | 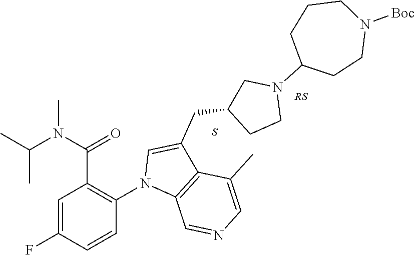 | Intermediate 225 & tert-butyl 4- oxoazepane-1-carboxylate |
| Compound 417 | 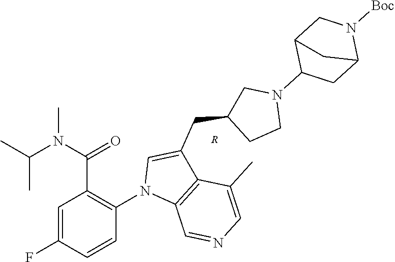 | Intermediate 202 & tert-butyl 5- oxo-2-azabicyclo[2.2.1]heptane- 2-carboxylate |
| Compound 418 | 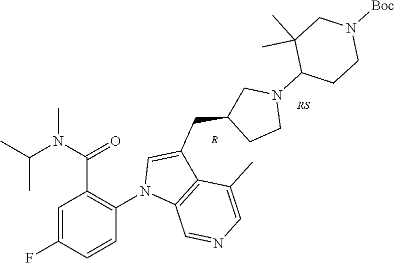 | Intermediate 202 & tert-butyl 3,3-dimethyl-4-oxopiperidine-1- carboxylate |
| Compound 419 | 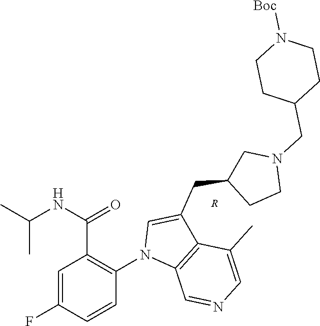 | Intermediate 319 & tert-butyl 4- formylpiperidine-1-carboxylate |
| Compound 420 & Compound 421 | 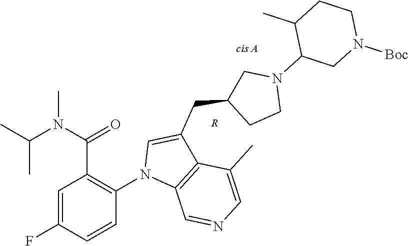 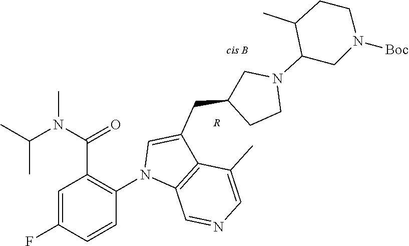 | Intermediate 202 & tert-butyl 4- methyl-3-oxopiperidine-1- carboxylate The product was separated by SFC (separation condition: DAICEL CHIRALPAK AD(250 mm*30 mm, 10 um); Mobile phase: A: Supercritical CO₂, B: 0.1% NH₃H₂O EtOH, A:B = 75:25 at 70 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 420 & the second fraction as Compound 421. |
| Compound 422 & Compound 423 | 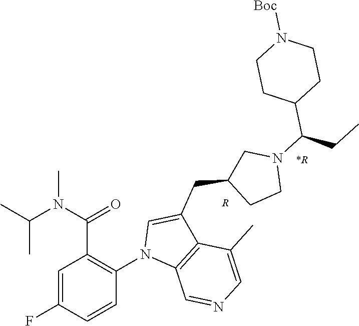 | Intermediate 202 & 327 The product was separated by SFC (separation condition: DAICEL CHIRALPAK IG (250 mm*30 mm, 10 um); Mobile phase: A: Supercritical CO₂, B: 0.1% NH₃H₂O IPA, A:B = 70:30 at 75 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 422 & the second fraction as Compound 423. |
| | 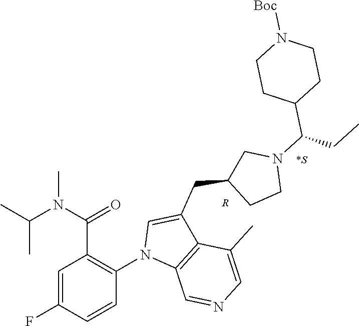 | |
| Intermediate 330 | 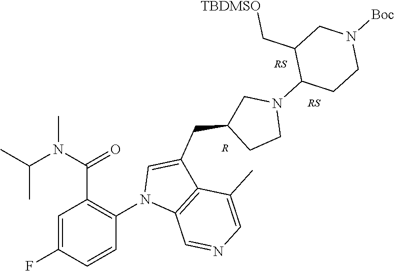 | Intermediate 202 & tert-butyl 3-(((tert- butyldimethylsilyl)oxy)methyl)- 4-oxopiperidine-1-carboxylate |
| Compound 424 | 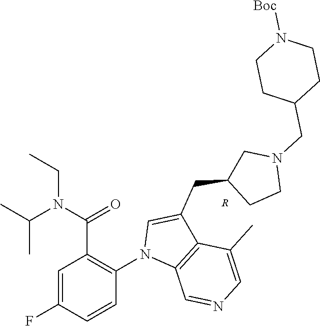 | Intermediate 338 & tert-butyl 4- formylpiperidine-1-carboxylate |
| Compound 425 | 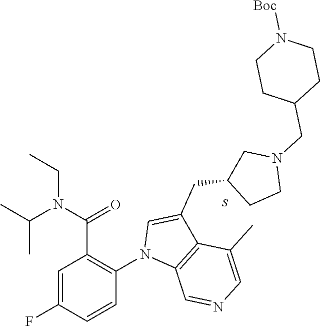 | Intermediate 342 & tert-butyl 4- formylpiperidine-1-carboxylate |
| Compound 426 | 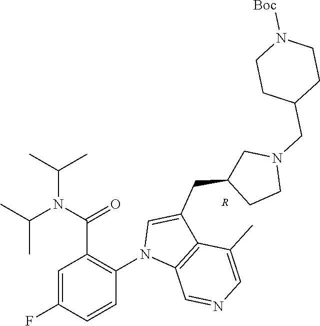 | Intermediate 347 & tert-butyl 4- formylpiperidine-1-carboxylate |
| Compound 427 | 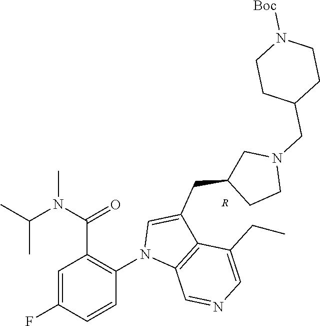 | Intermediate 354 & tert-butyl 4- formylpiperidine-1-carboxylate |
| Compound 428 | 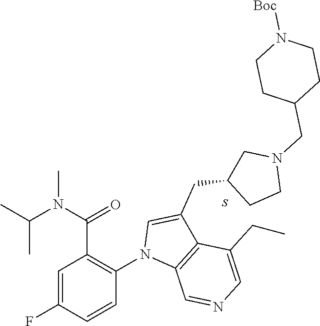 | Intermediate 358 & tert-butyl 4- formylpiperidine-1-carboxylate |
| Compound 527 | 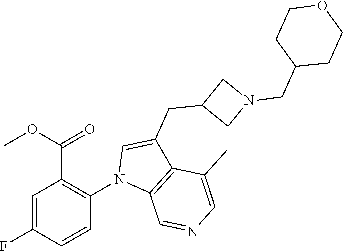 [SMILES: COC(=O)c1cc(F)ccc1-n1cc(CC2CN(CC3CCOCC3)C2)c2c(C)cncc21] | Intermediate 364 & tetrahydro- 2H-pyran-4-carbaldehyde |
| Compound 528 | 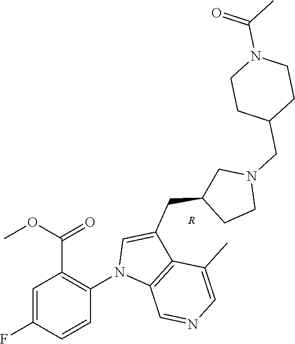 | Intermediate 368 & 1- acetylpiperidine-4-carbaldehyde |
| Compound 529 | 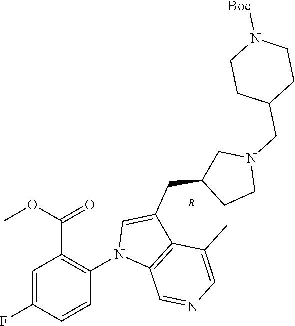 | Intermediate 368 & tert-butyl 4- formylpiperidine-1-carboxylate |
| Compound 531 | 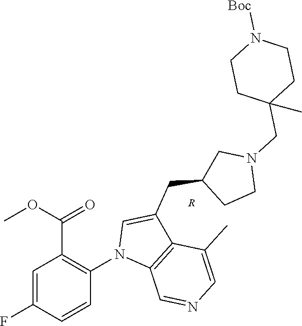 | Intermediate 368 & tert-butyl 4- formyl-4-methylpiperidine-1- carboxylate |
| Compound 532 | 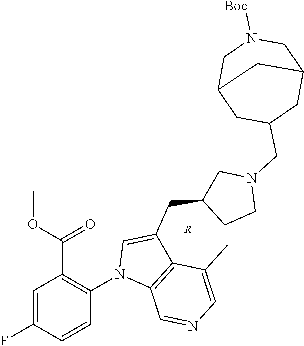 | Intermediate 368 & intermediate 398 |
| Compound 533 | 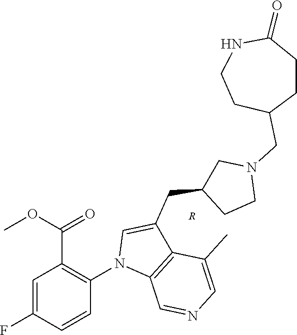 | Intermediate 368 & 7- oxoazepane-4-carbaldehyde |
| Compound 534 | 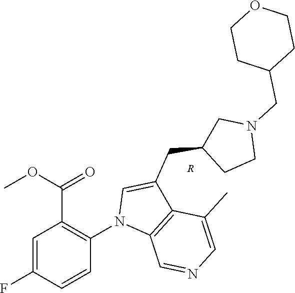 | Intermediate 368 & tetrahydro- 2H-pyran-4-carbaldehyde |

### Preparation of Compound 381:

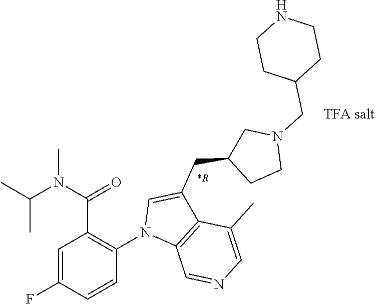 [SMILES: Cc1cncc2c1c(C[C@@H]1CCN(CC3CCNCC3)C1)cn2-c1ccc(F)cc1C(=O)N(C)C(C)C]

Compound 531 (70 mg, 0.104 mmol) was dissolved in DCM (3 mL, 46.837 mmol). TFA (1 mL, 13.067 mmol) was added. The mixture was stirred at RT for 2 hours. The solvent was removed to give Compound 381, which was used in the next step without further purification.

The Following Intermediates and Compounds were Synthesized by an Analogous Method as Described for Compound 381

| Int./Co No. | Structure | Starting Materials |
|---|---|---|
| Compound 429 | 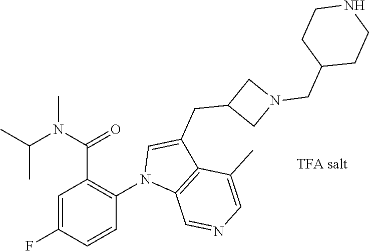 [SMILES: Cc1cncc2c1c(CC1CN(CC3CCNCC3)C1)cn2-c1ccc(F)cc1C(=O)N(C)C(C)C] | Compound 62 |
| Compound 430 | 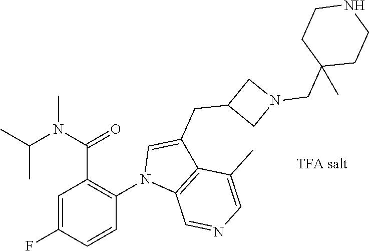 [SMILES: Cc1cncc2c1c(CC1CN(CC3(C)CCNCC3)C1)cn2-c1ccc(F)cc1C(=O)N(C)C(C)C] | Compound 382 |
| Compound 431 | 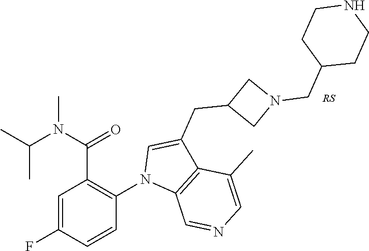 [SMILES: Cc1cncc2c1c(CC1CN(CC3CCNCC3)C1)cn2-c1ccc(F)cc1C(=O)N(C)C(C)C] | Compound 383 |
| Compound 432 | 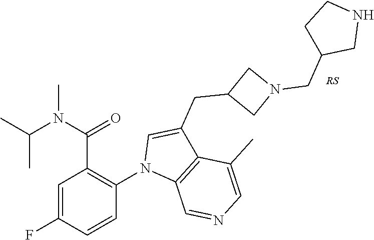 [SMILES: Cc1cncc2c1c(CC1CN(CC3CCNC3)C1)cn2-c1ccc(F)cc1C(=O)N(C)C(C)C] | Compound 384 |
| Compound 433 | 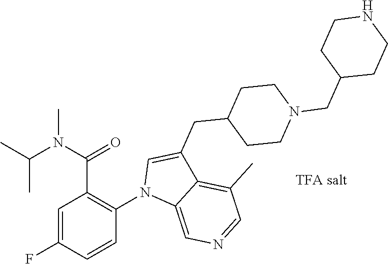 [SMILES: Cc1cncc2c1c(CC1CCN(CC3CCNCC3)CC1)cn2-c1ccc(F)cc1C(=O)N(C)C(C)C] | Compound 380 |
| Compound 434 | 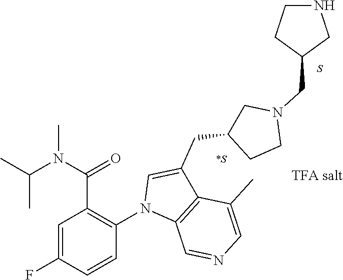 | Compound 491 |
| Compound 435 | 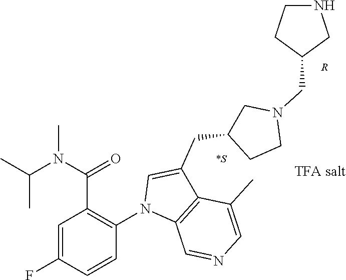 | Compound 492 |
| Compound 436 | 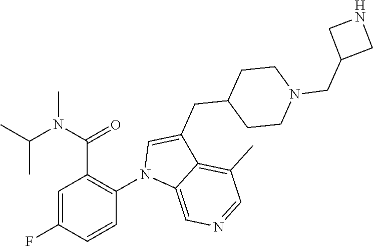 [SMILES: Cc1cncc2c1c(CC1CCN(CC3CNC3)CC1)cn2-c1ccc(F)cc1C(=O)N(C)C(C)C] | Compound 385 |
| Compound 437 | 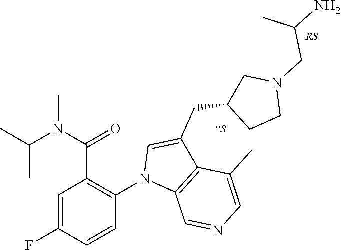 | Intermediate 157 |
| Compound 438 | 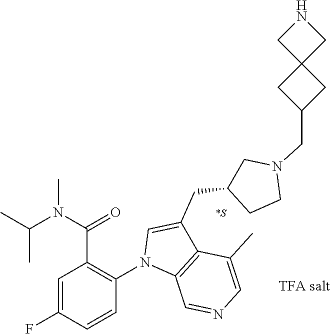 | Compound 386 |
| Compound 439 | 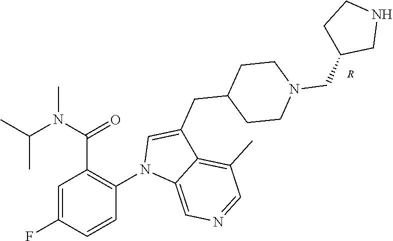 | Compound 493 |
| Compound 502 | 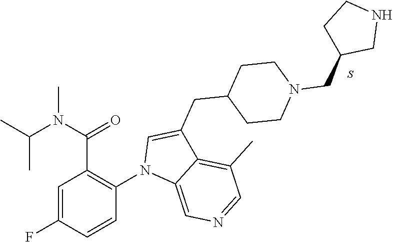 | Compound 440 |
| Compound 441 | 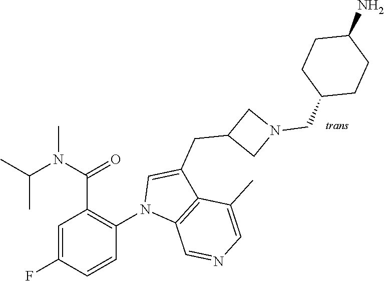 [SMILES: Cc1cncc2c1c(CC1CN(C[C@H]3CC[C@H](N)CC3)C1)cn2-c1ccc(F)cc1C(=O)N(C)C(C)C] | Compound 487 |
| Compound 442 | 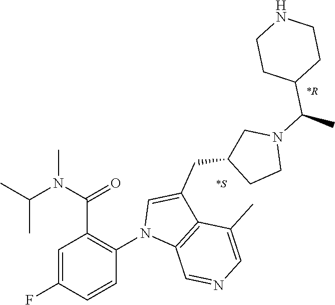 | Compound 387 |
| Compound 443 | 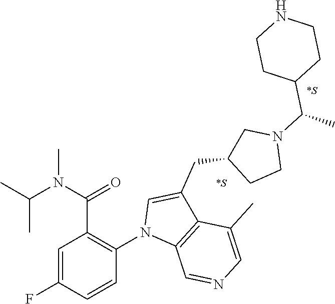 | Compound 388 |
| Compound 444 | 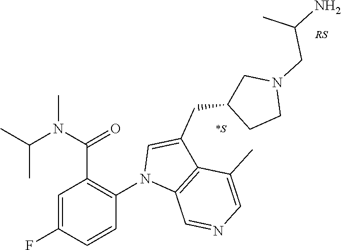 | Intermediate 171 |
| Compound 445 | 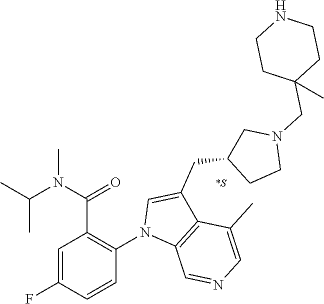 | Compound 389 |
| Compound 446 | 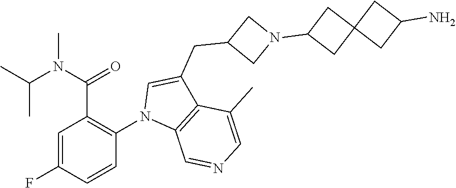 [SMILES: Cc1cncc2c1c(CC1CN(C3CC4(CC(N)C4)C3)C1)cn2-c1ccc(F)cc1C(=O)N(C)C(C)C] | Compound 91 |
| Compound 447 | 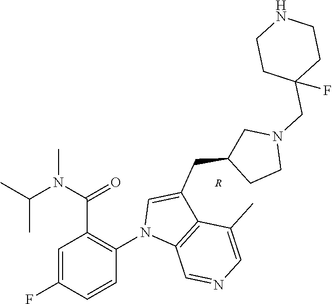 | Compound 392 |
| Compound 448 | 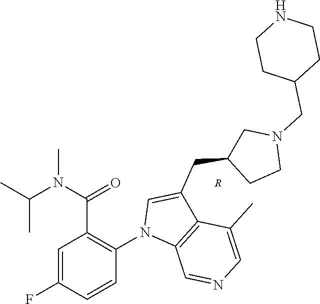 | Compound 394 |
| Compound 449 | 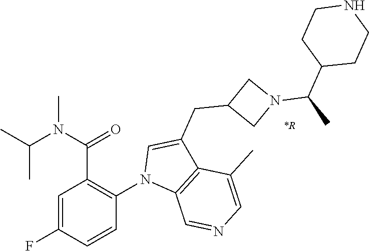 [SMILES: Cc1cncc2c1c(CC1CN([C@H](C)C3CCNCC3)C1)cn2-c1ccc(F)cc1C(=O)N(C)C(C)C] | Compound 395 |
| Compound 450 | 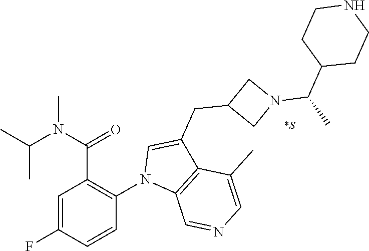 [SMILES: Cc1cncc2c1c(CC1CN([C@@H](C)C3CCNCC3)C1)cn2-c1ccc(F)cc1C(=O)N(C)C(C)C] | Compound 396 |
| Compound 451 | 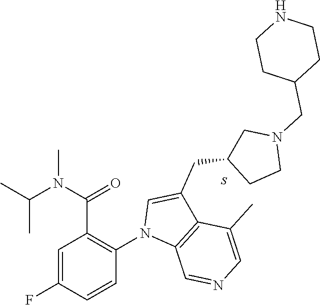 | Compound 397 |
| Compound 452 | 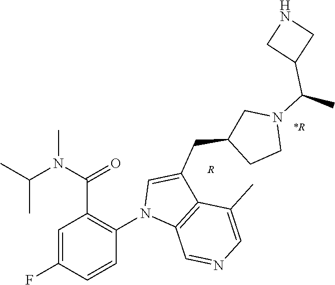 | Compound 398 |
| Compound 453 | 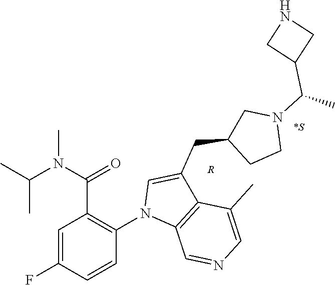 | Compound 399 |
| Compound 454 | 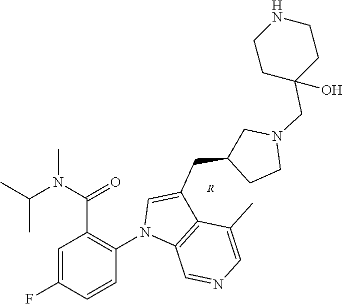 | Compound 500 |
| Compound 455 | 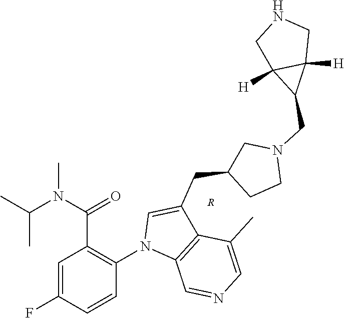 | Compound 495 |
| Compound 456 | 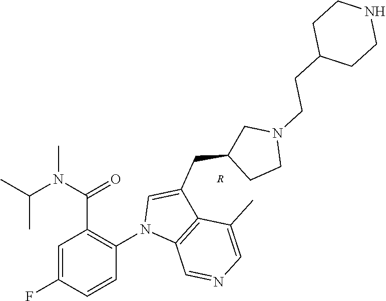 | Compound 400 |
| Compound 457 | 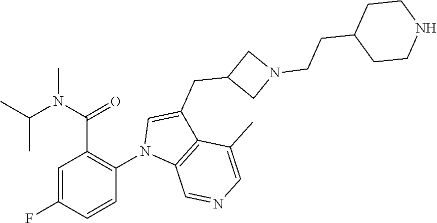 [SMILES: Cc1cncc2c1c(CC1CN(CCC3CCNCC3)C1)cn2-c1ccc(F)cc1C(=O)N(C)C(C)C] | Compound 401 |
| Compound 458 | 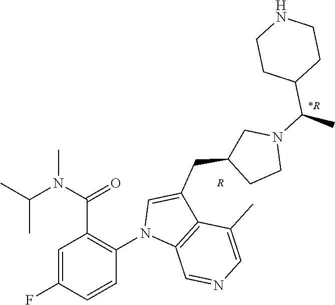 | Compound 402 |
| Compound 459 | 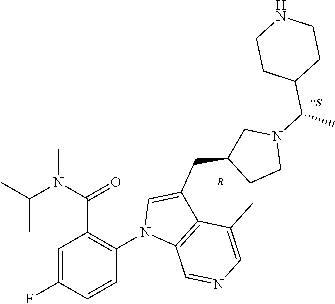 | Compound 403 |
| Compound 406a | 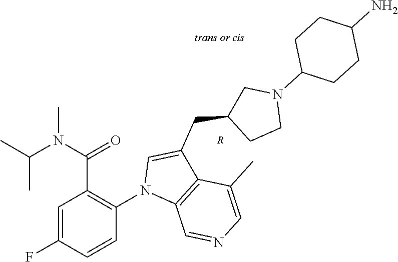 | Compound 404 |
| Compound 406b | 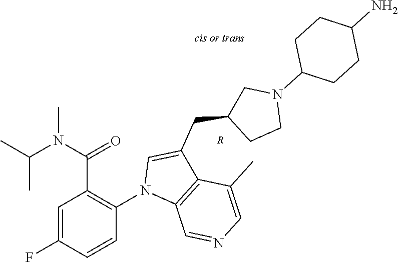 | Compound 405 |
| Compound 462 | 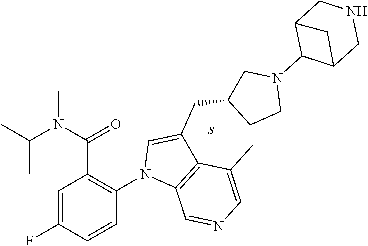 | Compound 408 |
| Compound 463 | 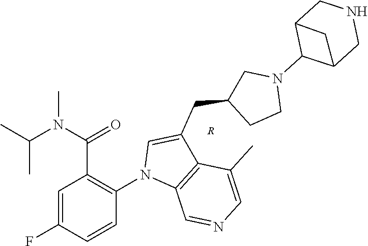 | Compound 409 |
| Compound 464 | 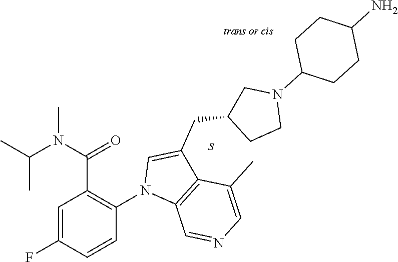 | Compound 410 |
| Compound 465 | 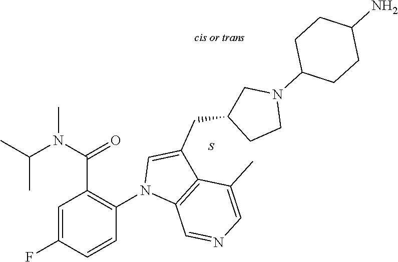 | Compound 411 |
| Compound 466 | 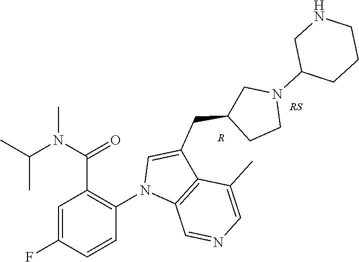 | Compound 412 |
| Compound 467 | 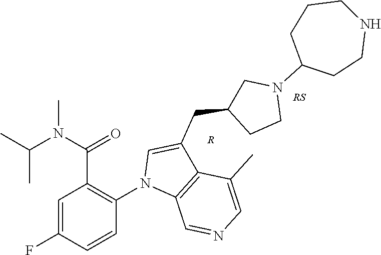 | Compound 413 |
| Compound 468 | 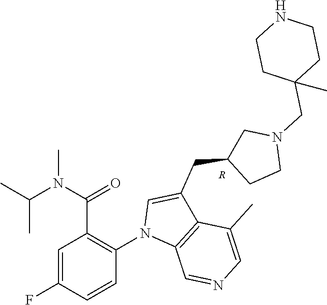 | Compound 414 |
| Compound 469 | 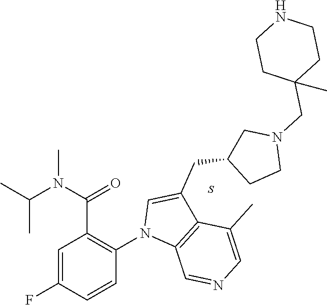 | Compound 415 |
| Compound 470 | 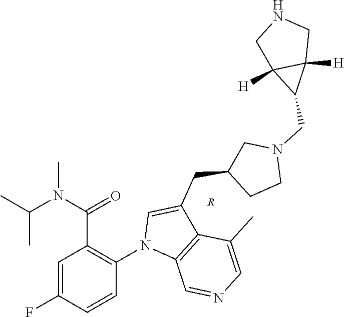 | Compound 496 |
| Compound 471 | 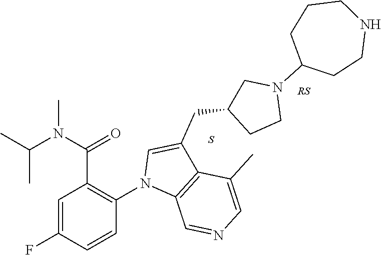 | Compound 416 |
| Compound 472 | 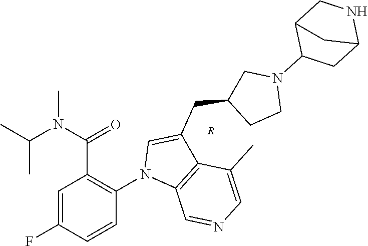 | Compound 417 |
| Compound 473 | 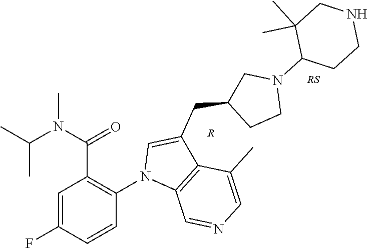 | Compound 418 |
| Compound 474 | 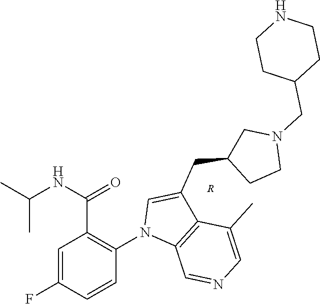 | Compound 419 |
| Compound 475 | 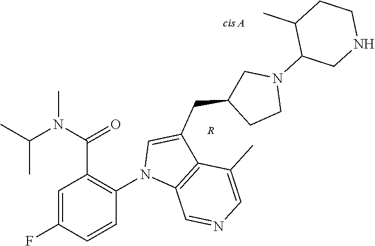 | Compound 420 |
| Compound 476 | 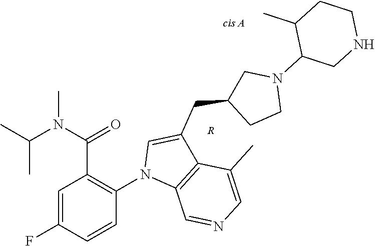 | Compound 421 |
| Compound 477 | 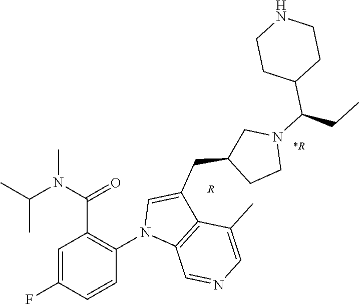 | Compound 422 |
| Compound 478 | 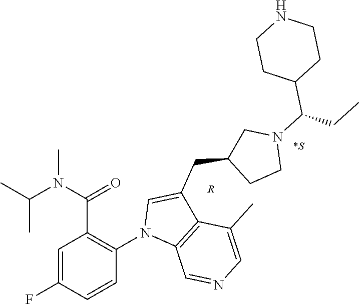 | Compound 423 |
| Compound 479 | 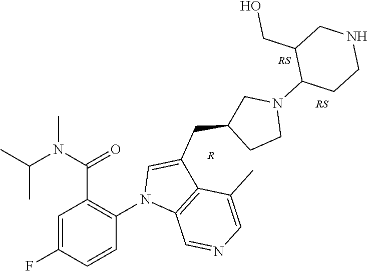 | Intermediate 330 |
| Compound 480 | 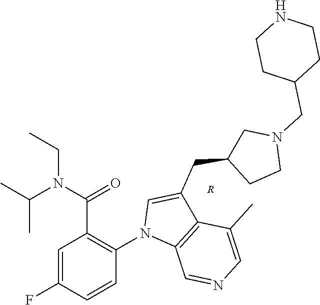 | Compound 424 |
| Compound 481 | 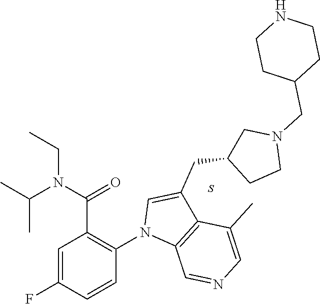 | Compound 425 |
| Compound 482 | 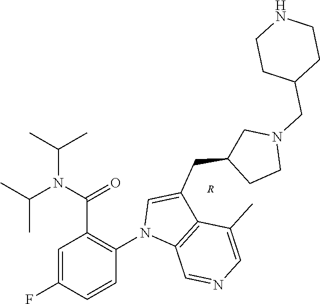 | Compound 426 |
| Compound 483 | 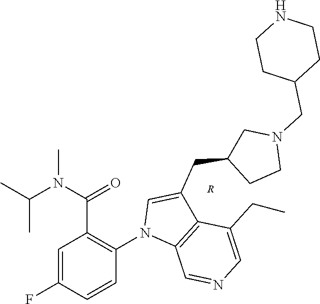 | Compound 427 |
| Compound 484 | 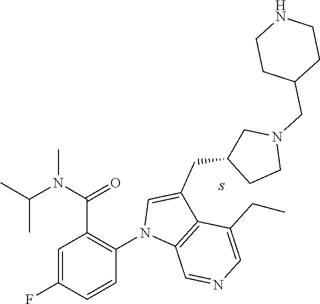 | Compound 428 |
| Compound 530 | 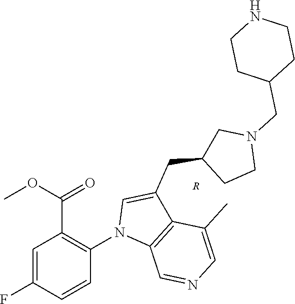 | Compound 529 |
| Intermediate 378 | 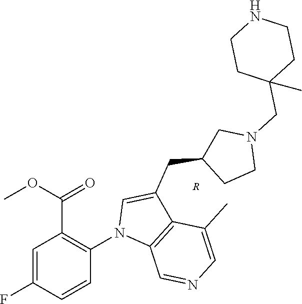 | Compound 531 |
| Intermediate 386 | 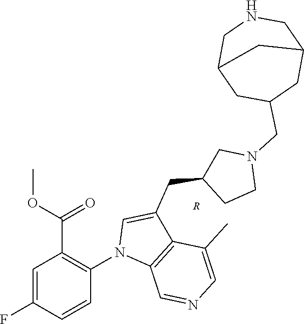 | Compound 532 |

### Preparation of Compound 485:

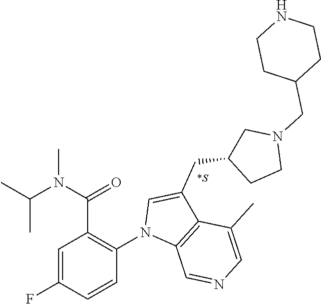 [SMILES: Cc1cncc2c1c(C[C@H]1CCN(CC3CCNCC3)C1)cn2-c1ccc(F)cc1C(=O)N(C)C(C)C]

At 0° C., to a solution of Compound 378 (650 mg, 95% purity, 1.02 mmol) in DCM (8 mL) was added hydrogen chloride in ethyl acetate (2.2 mL, 7 M). After stirring at room temperature for 2 hours, the reaction mixture was concentrated and the residue was basified with aqueous sodium hydroxide solution (1M) and extracted with DCM (20 mL) twice. The combined organic layers were washed with brine (20 mL), dried over Na₂SO₄, filtered and concentrated to afford Compound 485, which was used in the next step without purification.

### Preparation of Compound 486:

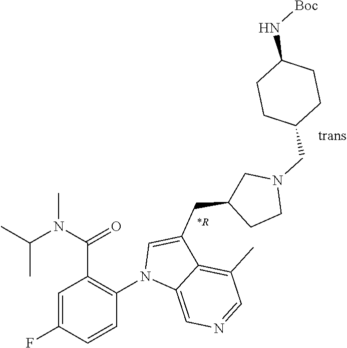 [SMILES: Cc1cncc2c1c(C[C@@H]1CCN(C[C@H]3CC[C@H](NC(=O)OC(C)(C)C)CC3)C1)cn2-c1ccc(F)cc1C(=O)N(C)C(C)C]

To a solution of intermediate 26 (300 mg, 0.734 mmol) and tert-butyl ((trans)-4-formylcyclohexyl)carbamate (334 mg, 1.47 mmol) in anhydrous methanol (8 mL) was added acetic acid (88.2 mg, 1.47 mmol). The reaction mixture was heated and stirred at 45° C. for 30 minutes before sodium cyanotrihydroborate (92.3 mg, 1.47 mmol) was added. After stirring at 45° C. for another 12 h, the reaction mixture was cooled down to room temperature, diluted with dichloromethane (50 mL), basified to pH=8 with saturated aq. sodium bicarbonate solution (40 mL) and extracted with dichloromethane (30 mL×3). The combined organic layers were dried over anhydrous Na₂SO₄, filtered and concentrated under reduced pressure to give a residue, which was purified by preparative-HPLC (Column: Boston Green ODS 150*30 mm*5 um, Mobile Phase A: water (0.225% FA), Mobile Phase B: acetonitrile, Flow rate: 35 mL/min, gradient condition from 1% B to 30% B). The pure fractions were collected and the solvent was evaporated under vacuum. The residue was partitioned between acetonitrile (2 mL) and water (10 mL). The mixture was lyophilized to dryness to give Compound 486 (400 mg, 92.5% purity, 81.3% yield) as a white powder.

### The Following Compound was Synthesized by an Analogous Method as Described for Compound 486

| Co No. | Structure | Starting Materials |
|---|---|---|
| Compound 487 | 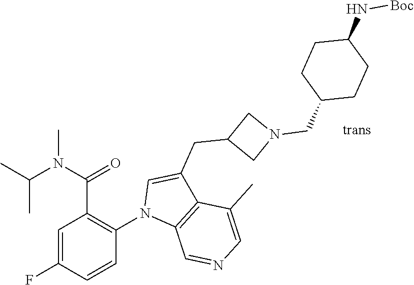 [SMILES: Cc1cncc2c1c(CC1CN(C[C@H]3CC[C@H](NC(=O)OC(C)(C)C)CC3)C1)cn2-c1ccc(F)cc1C(=O)N(C)C(C)C] | Intermediate 25 & tert-butyl ((trans)-4- formylcyclohexyl)carbamate |

### Preparation of Compound 488:

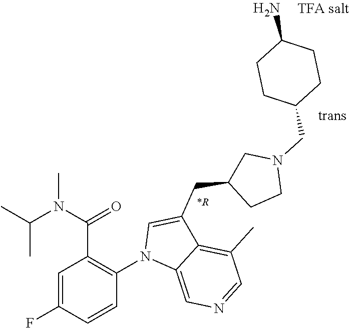 [SMILES: Cc1cncc2c1c(C[C@@H]1CCN(C[C@H]3CC[C@H](N)CC3)C1)cn2-c1ccc(F)cc1C(=O)N(C)C(C)C]

To a solution of Compound 486 (380 mg, 0.613 mmol) in anhydrous dichloromethane (4 mL) was added trifluoroacetic acid (4 mL). After stirring at 25° C. for 1 hour, the reaction mixture was concentrated under reduced pressure to give the Compound 488 (380 mg, crude, TFA salt) as a yellow oil.

### Preparation of Intermediate 114:

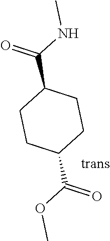

To a mixture of (trans)-4-(methoxycarbonyl)cyclohexanecarboxylic acid (5 g, 26.8 mmol), methanamine hydrochloride (2.72 g, 40.3 mmol), EDCI (6.2 g, 32.3 mmol), HOBt (6.0 g, 32.5 mmol) in DCM (80 mL) was added DIPEA (22.5 mL, 136 mmol). After stirring at room temperature overnight, the reaction mixture was diluted with DCM (50 mL), washed with 1 mol/L aq. HCl (100 mL), NaHCO₃ aq. (100 mL) and brine (100 mL) and dried over sodium sulfate. The solution was filtered and concentrated in vacuo to give intermediate 114 (4.4 g, 90% purity, 74.0% yield) as a white solid.

### Preparation of Intermediate 115:

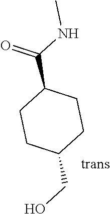

At 0° C., to a solution of LiAlH₄ (570 mg, 15.0 mmol) in dry THF (10 mL) under N₂, was added a solution of intermediate 114 (2.5 g, 12.5 mmol) in dry THF (20 mL) dropwise over 10 min. After addition, the reaction mixture was stirred at 0° C. for 2 h. The reaction was quenched with H₂O (0.5 mL), 10% aq. NaOH (0.5 mL), THF (10 mL), H₂O (1.5 mL), stirred for 10 min, and dried over Na₂SO₄. The suspension was filtered through Celite and the filtrate was concentrated to give crude intermediate 115 (1 g, 90% purity, 41.9% yield) as a white solid.

### Preparation of Intermediate 116:

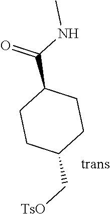 [SMILES: CNC(=O)[C@H]1CC[C@H](COS(=O)(=O)c2ccc(C)cc2)CC1]

At 0° C., to a solution of intermediate 115 (500 mg, crude) and triethylamine (1 ml, 7.20 mmol) in DCM (5 ml) was added a solution 4-methylbenzene-1-sulfonyl chloride (557 mg, 2.92 mmol) in DCM (5 ml) and N,N-dimethylpyridin-4-amine (71.5 mg, 0.585 mmol). After stirring at room temperature overnight, the reaction mixture was concentrated and the residue was purified by silica gel column chromatography eluting with methanol in dichloromethane from 0% to 9% to afford intermediate 116 (300 mg, 99.1% purity, 31.1% yield) as white solid.

The Following Intermediates were Synthesized by an Analogous Method as Described for Intermediate 116

| Int. No. | Structure | Starting Materials |
|---|---|---|
| Intermediate 207 | 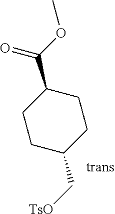 [SMILES: COC(=O)[C@H]1CC[C@H](COS(=O)(=O)c2ccc(C)cc2)CC1] | (trans)-methyl 4- (hydroxymethyl) cyclohexanecarboxylate |
| Intermediate 290 | 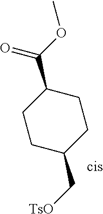 [SMILES: COC(=O)[C@H]1CC[C@@H](COS(=O)(=O)c2ccc(C)cc2)CC1] | (cis)-methyl 4- (hydroxymethyl) cyclohexanecarboxylate |

Preparation of intermediate 117:

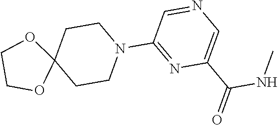 [SMILES: CNC(=O)c1cncc(N2CCC3(CC2)OCCO3)n1]

To a solution of 6-chloro-N-methylpyrazine-2-carboxamide (0.55 g, 3.2 mmol) in DMA (20 mL) was added 1,4-dioxa-8-azaspiro[4.5]decane (0.46 g, 3.2 mmol), followed by DIPEA (1.7 mL, 9.6 mmol). The mixture was heated up to 130° C. and stirred at that temperature overnight. After the reaction mixture was cooled down to ambient temperature, water and EtOAc were added. The layers were separated, and the water layer was extracted 3×more with EtOAc. The organic layers were combined, dried over Na₂SO₄, filtered, and evaporated to dryness. The residue was purified by silica gel column chromatography eluting with methanol in dichloromethane from 0% to 5% to give intermediate 117 (0.83 g, 3.0 mmol, yield: 93%) as an orange oil.

### Preparation of Intermediate 118:

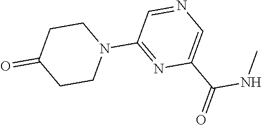 [SMILES: CNC(=O)c1cncc(N2CCC(=O)CC2)n1]

Intermediate 117 (830 mg, 3.0 mmol) was dissolved in THF (33 mL). To this solution was added 1M aq. HCl (33 mL) and the mixture was stirred at 50° C. until full consumption of the starting material (˜3 h). The mixture was cooled down to ambient temperature and sat. aq. NaHCO₃ solution and EtOAc were added. After separation of the layers, the water layer was extracted twice with EtOAc. The organic layers were combined, dried over Na₂SO₄, filtered, and evaporated to dryness. Purification by silica gel column chromatography eluting with methanol in dichloromethane from 0% to 4% gave intermediate 118 (0.40 g, 1.7 mmol, yield: 57%) as a yellow solid.

### Preparation of Intermediate 119:

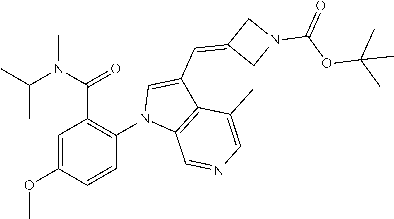 [SMILES: COc1ccc(-n2cc(C=C3CN(C(=O)OC(C)(C)C)C3)c3c(C)cncc32)c(C(=O)N(C)C(C)C)c1]

Intermediate 6 (0.40 g, 0.99 mmol), Cs₂CO₃ (1.09 g, 3.4 mmol), Pd(dppf)Cl₂ (0.072 g, 0.10 mmol) and t-butyl 3-methyleneazetidine-1-carboxylate (0.31 g, 1.8 mmol) were added to a flame dried vial, equipped with a stir bar. Next, the vial was evacuated and refilled with N₂, which was repeated three times. Then, anhydrous DMF (8.0 mL) was added, and the mixture stirred at 100° C. overnight. MeOH was added to dissolve the mixture and evaporated to dryness. The residue was partitioned between DCM/water. The layers were separated, and the water layer was extracted twice more with DCM. Organic layers were combined, dried over Na₂SO₄, filtered, and evaporated to dryness. Purification by silica gel column chromatography eluting with ethyl acetate in petroleum ether from 0% to 2.5% gave intermediate 119 (313 mg, 86% purity, 54% yield).

### Preparation of Intermediate 120:

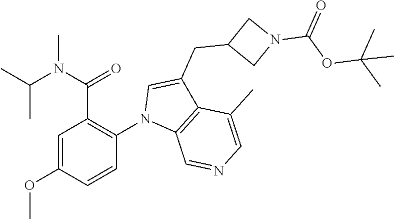 [SMILES: COc1ccc(-n2cc(CC3CN(C(=O)OC(C)(C)C)C3)c3c(C)cncc32)c(C(=O)N(C)C(C)C)c1]

To a mixture of intermediate 119 (0.31 g, 0.55 mmol) in MeOH (30 mL) was added a catalytic amount of Pd/C (10% w/w), and the solution was stirred under H₂ atmosphere with balloon for 2.5 h. Then, the mixture was filtered, washed with MeOH, and evaporated to dryness. The residue was purified by silica gel column chromatography eluting with ethyl acetate in heptane from 40% to 80% to give intermediate 120 (0.14 g, 0.29 mmol, 52% yield).

### Preparation of Intermediate 121:

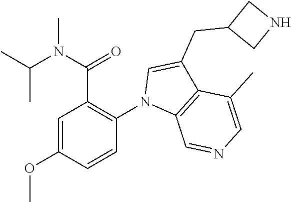 [SMILES: COc1ccc(-n2cc(CC3CNC3)c3c(C)cncc32)c(C(=O)N(C)C(C)C)c1]

Dissolve intermediate 120 (0.14 g, 0.28 mmol) in DCM (3 mL). Next, TFA (3 mL) was added. The mixture was subsequently stirred at ambient temperature for ˜3 h. Then, the mixture was evaporated to dryness, and applied to a SiliaBond@ propylsulfonic acid resin as a solution in MeOH. The resin was eluted with MeOH (7 fractions), followed by 3.5N NH₃ in MeOH (7 fractions). Product containing fractions were pooled and evaporated to dryness to give intermediate 121 (0.11 g, 0.26 mmol), which was used in the next step without further purification.

### Preparation of Intermediate 122:

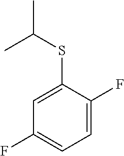

2-Iodopropane (1.64 mL, 16.4 mmol) was added at r.t. to a solution of 2,5-difluorothiophenol (2.00 g, 13.7 mmol) and potassium carbonate (2.65 g, 19.2 mmol) in acetone (46 mL) and the reaction mixture was stirred at 75° C. for 2 h. The reaction mixture was cooled back to r.t., quenched with water (20 mL) and concentrated under reduced pressure to remove the acetone. The aqueous layer was extracted with DCM (4×40 mL) and the combined organic layers were washed with brine (100 mL), dried over Na₂SO₄, filtered and concentrated under reduced pressure to give intermediate 122 (2.35 g, 91.2% yield) as a pale yellow oil which was used without further purification in the following step.

### Preparation of Intermediate 123:

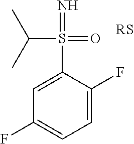

Iodobenzene diacetate (PIDA) (8.16 g, 25.3 mmol) was added at rt to a stirred solution of intermediate 122 (2.34 g, 12.1 mmol) and ammonium carbamate (1.41 g, 18.1 mmol) in MeOH (24 mL) and the reaction mixture was stirred at rt overnight. The reaction mixture was concentrated under reduced pressure to afford a yellow mixture. The crude product was purified by silica gel column chromatography eluting with ethyl acetate in heptane from 10% to 100% to give intermediate 123 (2.45 g, yield 93%) as a colorless oil.

### Preparation of Intermediate 124:

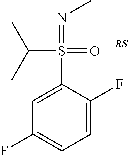

Mel (1.04 mL, 16.8 mmol) was added under nitrogen to a mixture of intermediate 123 (2.45 g, 11.2 mmol) and KOH (1.25 g, 22.4 mmol) in DMSO (61 mL) and the reaction mixture was stirred at r.t. for 90 min. The mixture was diluted with water (600 mL), extracted with ethyl acetate (×4), and the combined organic phases were dried over sodium sulfate, filtered and evaporated to dryness. The crude product was purified by silica gel column chromatography eluting with ethyl acetate in heptane from 30% to 100% to give intermediate 124 (2.41 g, 92.4% yield) as a pale yellow oil.

### Preparation of Intermediate 125:

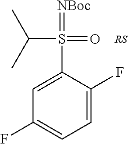

t-BuOK 1.0 M in THF (5.7 mL, 5.70 mmol) was added under nitrogen at 0° C. to a stirred solution of intermediate 123 (1.00 g, 4.56 mmol) in anhydrous THF (15 mL). After 15 min, BOC-anhydride (1.99 g, 9.12 mmol) in anhydrous THF (30 mL) was added and the reaction was left under stirring at rt for 60 h. The reaction was evaporated to dryness, and the residue was dissolved in EtOAc, and the organic phase was washed with water (×2), dried over sodium sulfate, filtered, and evaporated to dryness. The crude product was purified by silica gel column chromatography eluting with ethyl acetate in heptane from 10% to 80% to give intermediate 125 (1.5 g, 99.9% yield) as a white solid.

### Preparation of Intermediate 126:

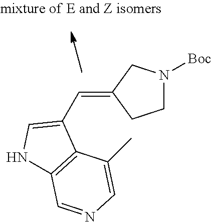 [SMILES: Cc1cncc2[nH]cc(/C=C3\CCN(C(=O)OC(C)(C)C)C3)c12]

t-Butyl 3-formylpyrrolidine-1-carboxylate (4.15 g, 20.8 mmol) was added to a stirred mixture of intermediate 1 (2.50 g, 18.9 mmol) and NaOH (2.27 g, 56.7 mmol) in MeOH (77 mL), and the solution was refluxed for 26 h. The reaction was evaporated to dryness and purified by silica gel column chromatography eluting with methanol in dichloromethane from 2% to 20% to give intermediate 126 as an E & Z mixture (6.11 g, yield 62.9%, 61% purity) which was used in the following step without any further purification.

### The Following Intermediate was Synthesized by an Analogous Method as Described for Intermediate 126

| Int. No. | Structure | Starting Materials |
|---|---|---|
| Intermediate 237 | 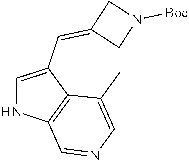 [SMILES: Cc1cncc2[nH]cc(C=C3CN(C(=O)OC(C)(C)C)C3)c12] | Intermediate 1 & t-butyl 3- formylazetidine- 1-carboxylate |

### Preparation of Intermediate 127:

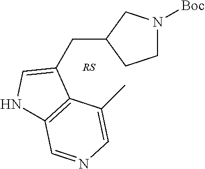 [SMILES: Cc1cncc2[nH]cc(CC3CCN(C(=O)OC(C)(C)C)C3)c12]

To a solution of intermediate 126 (6.10 g, 19.5 mmol) in MeOH (79 mL) was added Pd/C (10% w/w) (1.90 g, 1.78 mmol) under nitrogen. The suspension was hydrogenated at 1 bar Hydrogen at rt for 16 h. The reaction was filtered over Celite®, and the filtrate was evaporated to dryness. The residue was purified by reversed-phase prep. HPLC purification (Stationary phase: RP Xbridge Prep C18 OBD-5 μm, 50×250 mm, Mobile phase: 0.5% NH₄HCO₃ solution in water, CH₃CN) to give intermediate 127 (1.71 g, yield 45.7%) as a white solid.

### The Following Intermediate was Synthesized by an Analogous Method as Described for Intermediate 127

| Int. No. | Structure | Starting Materials |
|---|---|---|
| Intermediate 238 | 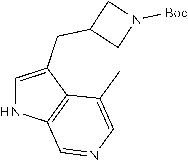 [SMILES: Cc1cncc2[nH]cc(CC3CN(C(=O)OC(C)(C)C)C3)c12] | Intermediate 237 |

### Preparation of Intermediate 128:

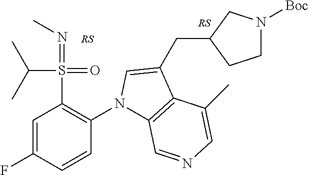 [SMILES: CN=S(=O)(c1cc(F)ccc1-n1cc(CC2CCN(C(=O)OC(C)(C)C)C2)c2c(C)cncc21)C(C)C]

Potassium tert-butoxide (0.42 mL, 1 M in THF, 0.418 mmol) was added under nitrogen to a solution of intermediate 127 (110 mg, 0.350 mmol) in anhydrous dioxane (1.7 mL). After 10 min, this solution was added to a solution of intermediate 124 (203 mg, 0.872 mmol) in anhydrous dioxane (1.5 mL), and the mixture was stirred at 80 SC overnight. The reaction was cooled down to rt, evaporated to dryness, and the crude was purified by silica gel column chromatography eluting with methanol in dichloromethane from 000 to 15% to give intermediate 128 (96 mg, yield 35.4%) as a yellow glassy solid.

The Following Intermediates were Synthesized by an Analogous Method as Described for Intermediate 128

| Int. No. | Structure | Starting Materials |
|---|---|---|
| Intermediate 129 | 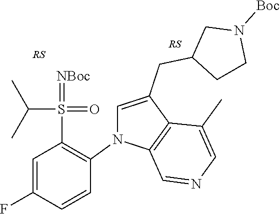 | Intermediate 125 & 127 |
| Intermediate 239 | 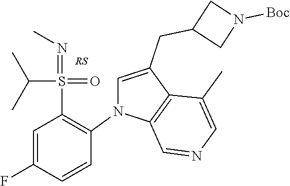 [SMILES: CN=S(=O)(c1cc(F)ccc1-n1cc(CC2CN(C(=O)OC(C)(C)C)C2)c2c(C)cncc21)C(C)C] | Intermediate 238 & 124 |
| Intermediate 241 | 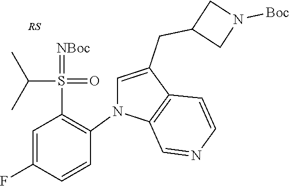 | Intermediate 238 & 125 |
| Intermediate 277 | 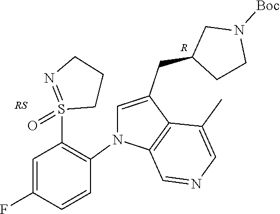 | Intermediate 140a & 276 |
| Intermediate 297 | 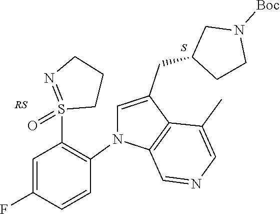 | Intermediate 140b & 276 |
| Intermediate 309 | 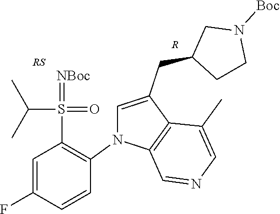 | intermediate 125 & 140a |
| Intermediate 310 | 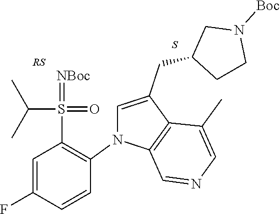 | intermediate 125 & 140b |

### Preparation of Intermediate 130:

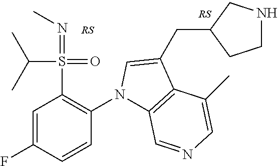 [SMILES: CN=S(=O)(c1cc(F)ccc1-n1cc(CC2CCNC2)c2c(C)cncc21)C(C)C]

TFA (1.01 mL, 13.2 mmol) was added to a stirred solution of intermediate 128 (97.0 mg, 0.183 mmol) in DCM (1.0 mL). After 30 min, the reaction was evaporated to dryness, and the crude product was dissolved in MeOH and transferred to a column loaded with SiliaBond® propylsulfonic acid resin resin. The column was first eluted with MeOH (20 mL), followed by NH₃ in methanol (7N, 12 mL). The tubes containing the product were concentrated under reduced pressure to give intermediate 130 (78 mg, yield 66.5%) as a pale yellow glassy solid.

The Following Intermediates were Synthesized by an Analogous Method as Described for Intermediate 130

| Int. No. | Structure | Starting Materials |
|---|---|---|
| Intermediate 131 | 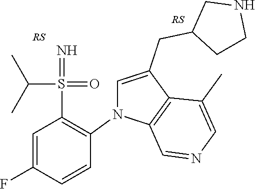 | Intermediate 129 |
| Intermediate 131a & Intermediate 131b & Intermediate 131c & Intermediate 131d | 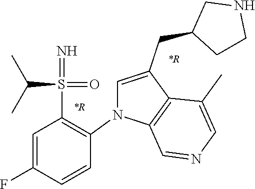 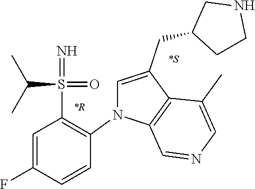 | Intermediate 131 was separated via Prep SFC (Stationary phase: Chiralpak Diacel AD 20 × 250 mm, Mobile phase: CO₂, EtOH- iPrOH (50-50) + 0.4% iPrNH₂). The first fraction was collected as intermediate 131a, the second fraction as intermediate 131b and the third fraction which was a mixture of intermediate 131c & 131d was further separated by Prep SFC (Stationary phase: Chiralcel Diacel IH 20 × 250 mm, Mobile phase: CO₂, EtOH + 0.4 iPrNH₂). The first fraction was collected as intermediate 131c and the second fraction as intermediate 131d |
| | 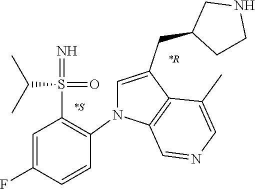 | |
| | 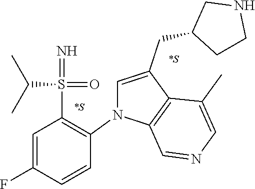 | |
| Intermediate 240 | 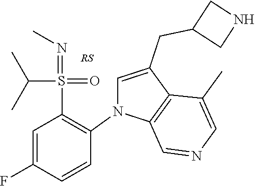 [SMILES: CN=S(=O)(c1cc(F)ccc1-n1cc(CC2CNC2)c2c(C)cncc21)C(C)C] | Intermediate 239 |
| Intermediate 242 | 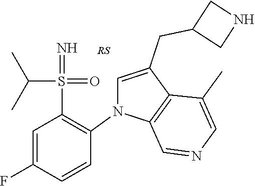 [SMILES: Cc1cncc2c1c(CC1CNC1)cn2-c1ccc(F)cc1S(=N)(=O)C(C)C] | Intermediate 241 |
| Intermediate 278 | 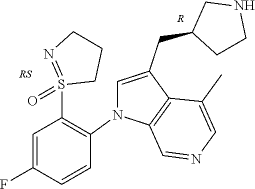 | Intermediate 277 |
| Intermediate 298 | 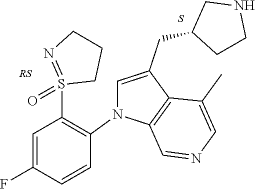 | Intermediate 297 |
| Intermediate 311a & Intermediate 311b | 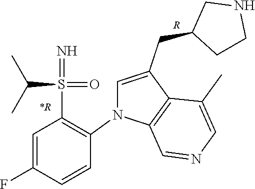 | Intermediate 309 The product was separated by Prep SFC (Stationary phase: Chiralpak Diacel AD 20 × 250 mm, Mobile phase: CO₂, EtOH + 0.4 iPrNH₂). The first fraction was collected as intermediate 311a & the second fraction as intermediate 311b. |
| | 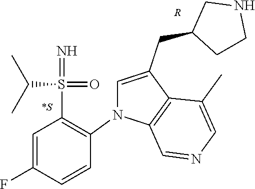 | |
| Intermediate 312a & Intermediate 312b | 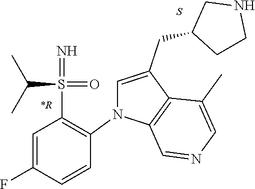 | Intermediate 310 The product was separated by Prep SFC (Stationary phase: Chiralpak Diacel AD 20 × 250 mm, Mobile phase: CO₂, EtOH + 0.4 iPrNH₂). The first fraction was collected as intermediate 312a & the second fraction as intermediate 312b. |
| | 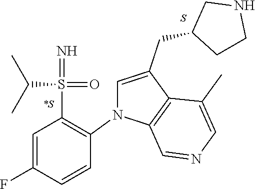 | |

### Preparation of Intermediate 134:

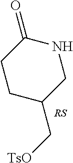 [SMILES: Cc1ccc(S(=O)(=O)OCC2CCC(=O)NC2)cc1]

To a solution of 5-(hydroxymethyl)piperidin-2-one (300 mg, 2.32 mmol) in DMF (5 mL), was added sodium hydride (60% in mineral oil) (140 mg, 3.484 mmol) at 0° C. After 10 min, 4-methylbenzene-1-sulfonyl chloride (532 mg, 2.787 mmol) was added and the mixture was stirred at 0° C. for 3 hr. The mixture was quenched by water (30 mL) and extracted with EtOAc (20 mL×3). The combined organic layers were washed with brine, dried over Na₂SO₄, filtered and concentrated to get the crude, which was purified by silica gel column chromatography eluting with MeOH in DCM from 0% to 10% to give intermediate 134 (217 mg, 89.15% purity from LCMS, 29.4% yield) as a white solid.

### Preparation of Intermediate 135:

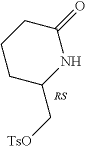 [SMILES: Cc1ccc(S(=O)(=O)OCC2CCCC(=O)N2)cc1]

At 0° C., to a solution of 6-(hydroxymethyl)piperidin-2-one (180 mg, 1.39 mmol), DIEA (0.48 mL, 2.8 mmol) and 4-dimethylaminopyridine (17.0 mg, 0.14 mmol) in DCM (10.8 mL) was added 4-methylbenzenesulfonyl chloride (433.2 mg, 2.27 mmol). After stirring at r.t. for 16 hours, the resulting mixture was washed with brine, drived over Na₂SO₄. The organic solvent was removed and the residue was purified by silica gel column chromatography eluting with ethyl acetate in petroleum ether from 50% to 100% to afford intermediate 135 (300 mg, 90% purity, 68% yield).

The Following Intermediates were Synthesized by an Analogous Method as Described for Intermediate 135

| Int. No. | Structure | Starting Materials |
|---|---|---|
| Intermediate 149 | 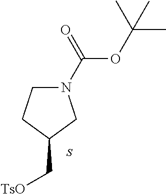 | tert-butyl (S)-3- (hydroxymethyl) pyrrolidine-1- carboxylate |
| Intermediate 150 | 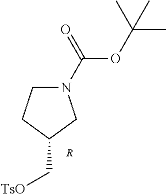 | tert-butyl (R)-3- (hydroxymethyl) pyrrolidine-1- carboxylate |
| Intermediate 159 | 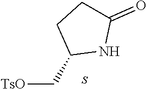 | (S)-5- (hydroxymethyl)- 205-yrrolidine- 2-one |
| Intermediate 162 | 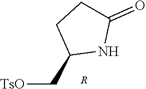 | (R)-5- (hydroxymethyl)- 205-yrrolidine- 2-one |
| Intermediate 177 | 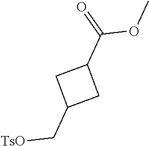 [SMILES: COC(=O)C1CC(COS(=O)(=O)c2ccc(C)cc2)C1] | methyl 3- (hydroxymethyl) cyclobutanecarboxylate |
| Intermediate 249 | 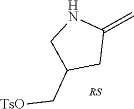 [SMILES: C=C1CC(COS(=O)(=O)c2ccc(C)cc2)CN1] | 4-(hydroxymethyl)- pyrrolidine- 2-one |
| Intermediate 250 | 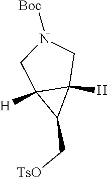 [SMILES: Cc1ccc(S(=O)(=O)OC[C@@H]2[C@H]3CN(C(=O)OC(C)(C)C)C[C@@H]23)cc1] | (1R,5S,6r)-tert-butyl 6- (hydroxymethyl)-3- azabicyclo[3.1.0]hexane- 3- carboxylate |
| Intermediate 273 | 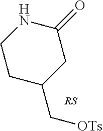 [SMILES: Cc1ccc(S(=O)(=O)OCC2CCNC(=O)C2)cc1] | 4-(hydroxymethyl) piperidin- 2- one |
| Intermediate 287 | 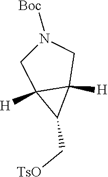 [SMILES: Cc1ccc(S(=O)(=O)OC[C@H]2[C@H]3CN(C(=O)OC(C)(C)C)C[C@@H]23)cc1] | tert-butyl (1R,5S,6s)-6- (hydroxymethyl)-3- azabicyclo[3.1.0]hexane-3- carboxylate |
| Intermediate 336 | 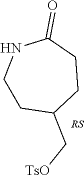 [SMILES: Cc1ccc(S(=O)(=O)OCC2CCNC(=O)CC2)cc1] | 5-(hydroxymethyl)azepan- 2-one |

### Preparation of Intermediate 138:

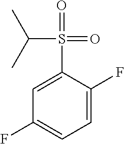

HCl in water (0.376 mL, 0.1 M, 0.038 mmol) was added to a stirred solution of intermediate 123 (550 mg, 2.51 mmol) and m-CPBA (1.237 g, 5.02 mmol) in THF (9.8 mL). The mixture was heated at reflux for 24 h. The mixture was cooled down to rt, diluted with EtOAc and washed with NaOH 1N (×3), water (×1), sat Na₂S₂O₃ (×3), dried over sodium sulfate, filtered and evaporated to dryness. The crude was purified by by silica gel column chromatography eluting with ethyl acetate in petroleum ether from 10% to 70% to give intermediate 138 (300 mg, yield 54.3%) as a colorless oil.

### Preparation of Intermediate 127, 140a & 140b:

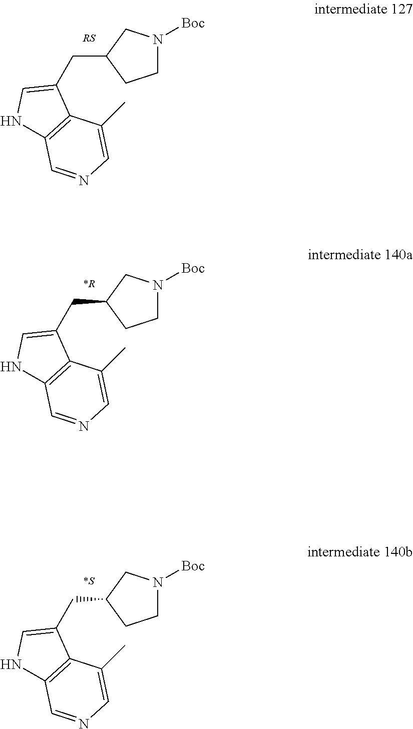 [SMILES: Cc1cncc2[nH]cc(CC3CCN(C(=O)OC(C)(C)C)C3)c12 ; Cc1cncc2[nH]cc(C[C@@H]3CCN(C(=O)OC(C)(C)C)C3)c12 ; Cc1cncc2[nH]cc(C[C@H]3CCN(C(=O)OC(C)(C)C)C3)c12]

Intermediate 126 (6.10 g, 11.9 mmol) was dissolved in MeOH (48 mL) and added under nitrogen to Pd/C (10% w/w) (1.90 g, 1.78 mmol), and the mixture was hydrogenated at 1 bar hydrogen at r.t. during 16 h. The reaction was filtered over Celite®, evaporated to dryness to afford 5.3 g of crude product. 400 mg of crude product was purified by prep HPLC and the remaining was purified by reversed-phase prep HPLC purification (Stationary phase: RP Xbridge Prep C18 OBD-5 μm, 50×250 mm, Mobile phase: 0.5% NH₄HCO₃ solution in water, CH₃CN) to give intermediate 127 (1.58 g, yield 42.3%) as white solid. A further purification was performed via Prep SFC (Stationary phase: Chiralpak Diacel AD 20×250 mm, Mobile phase: CO₂, iPrOH+0.4 iPrNH₂). The first fraction was collected as intermediate 140a and the second fraction as intermediate 140b.

### Preparation of Intermediate 141:

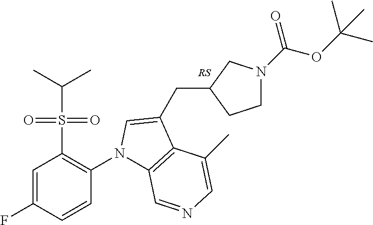 [SMILES: Cc1cncc2c1c(CC1CCN(C(=O)OC(C)(C)C)C1)cn2-c1ccc(F)cc1S(=O)(=O)C(C)C]

Potassium tert-butoxide (0.68 mL, 1 M in THF, 0.681 mmol) was added under nitrogen to a solution of intermediate 127 (179 mg, 0.568 mmol) in anhydrous dioxane (2.6 mL) at rt. After 10 min, this solution was added to a solution of intermediate 138 (300 mg, 1.36 mmol) in dioxane (2.6 mL). The mixture was stirred at 80° C. overnight. The mixture was evaporated to dryness and purified by by silica gel column chromatography eluting with methanol in dichloromethane from 2% to 15% to give intermediate 141 (145 mg, yield 49.5%) as a pale yellow solid.

### Preparation of Intermediate 142:

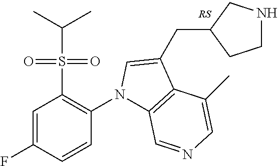 [SMILES: Cc1cncc2c1c(CC1CCNC1)cn2-c1ccc(F)cc1S(=O)(=O)C(C)C]

TFA (1.5 mL, 20.3 mmol) was added to a stirred solution of intermediate 142 (145 mg, 0.281 mmol) in DCM (1.5 mL). After 30 min the reaction was evaporated to dryness, and the crude product was dissolved in MeOH and added to a column loaded with SiliaBond® propylsulfonic acid resin. The column was first eluted with MeOH (10 mL), followed by NH₃ in MeOH (7 N, 5 mL). The tubes containing the product were concentrated under reduced pressure to give intermediate 142 (99 mg, yield 84.7%) as a yellow solid.

### Preparation of Compound 489:

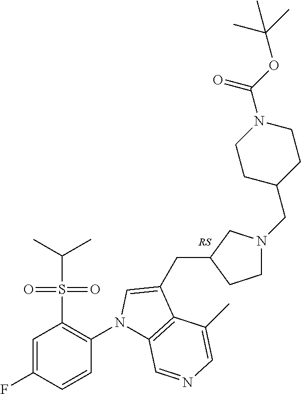 [SMILES: Cc1cncc2c1c(CC1CCN(CC3CCN(C(=O)OC(C)(C)C)CC3)C1)cn2-c1ccc(F)cc1S(=O)(=O)C(C)C]

A mixture of intermediate 142 (50 mg, 0.12 mmol) and tert-butyl 4-formylpiperidine-1-carboxylate (51.3 mg, 0.241 mmol) in MeOH (1.20 mL) was stirred for 30 min after which sodium cyanoborohydride (15.1 mg, 0.241 mmol) was added. The reaction mixture was stirred at rt for 2 h, after which it was quenched with water. The mixture was purified on a column loaded with SiliaBond® propylsulfonic acid resin. The column was first eluted with MeOH (10 mL), followed by NH₃ in MeOH (7 N, 4 mL). The tubes containing the product were concentrated under reduced pressure to give Compound 489 (64 mg, yield 79%) as a yellow solid.

### Preparation of Compound 490:

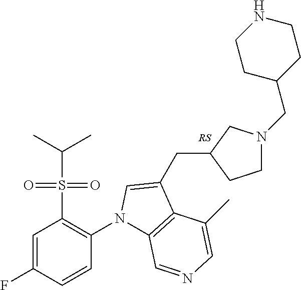 [SMILES: Cc1cncc2c1c(CC1CCN(CC3CCNCC3)C1)cn2-c1ccc(F)cc1S(=O)(=O)C(C)C]

TFA (0.57 mL, 7.40 mmol) was added to a stirred solution of Compound 489 (63 mg, 0.10 mmol) in DCM (0.58 mL). After 30 min the reaction was evaporated to dryness, and the crude product was dissolved in MeOH (2 mL), stirred for 30 min and added to a column loaded with SiliaMetS® Diamine resin, filtered and evaporated to dryness to give Compound 490 (55 mg, quantitative yield) as a yellow solid.

### Preparation of Intermediate 147:

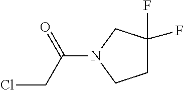 [SMILES: O=C(CCl)N1CCC(F)(F)C1]

3,3-difluoropyrrolidine. HCl (0.30 g, 2.1 mmol) was suspended in DCM (10 mL). Next, the mixture was cooled to 0° C. in an ice bath. Then, triethylamine (0.73 mL, 5.2 mmol) was added and the mixture stirred at 0° C. for −5 min. Next, chloroacetylchloride (0.18 mL, 2.2 mmol) was added dropwise. The resulting mixture was stirred at 0° C. for ˜1 h, after which water was added. Then, the mixture was stirred for an additional 5 min, after which it was transferred to a separatory funnel. Next, 1M aq. HCl solution was added and the layers were separated. The organic layer was dried over Na₂SO₄, filtered and evaporated to dryness to give intermediate 147 as a dark coloured oil (0.29 g, yield 76%).

### The Following Intermediate was Synthesized by an Analogous Method as Described for Intermediate 147

| Int. No. | Structure | Starting Materials |
|---|---|---|
| Intermediate 148 | 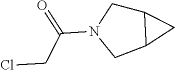 | 3-azabicyclo [3.1.0]hexane.HCl |

### Preparation of Compound 491:

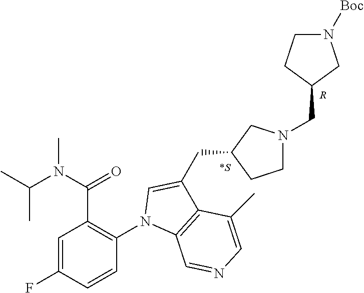

To a mixture of intermediate 27 (70 mg, 0.171 mmol) in NMP (3 mL) was added intermediate 149 (182.7 mg, 0.51 mmol), DIEA (0.088 mL, 0.51 mmol) and potassium iodide (28.4 mg, 0.17 mmol) at rt. Then the mixture continued to stir for 6 h at 80° C. The mixture was diluted by water and extracted with DCM three times. The combined organic layers were dried over Na₂SO₄, filtered and concentrated. The residue was purified by RP silica gel column chromatography eluting with MeCN in water with 0.05% formic acid from 5% to 95% to afford Compound 491 (50 mg, 49% yield) as a yellow oil.

The Following Compounds were Synthesized by an Analogous Method as Described for Compound 491

| Co No. | Structure | Starting Materials |
|---|---|---|
| Compound 492 | 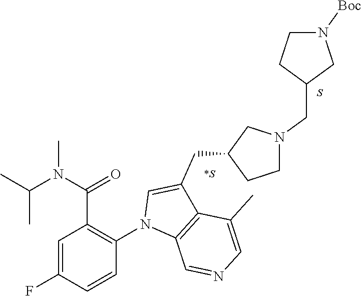 | Intermediate 27 & 150 |
| Compound 493 | 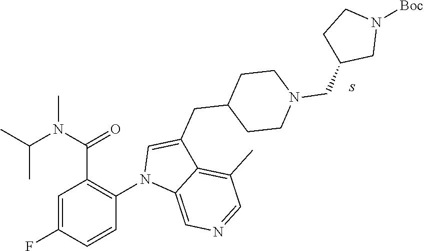 | Intermediate 28 & 150 |
| Compound 440 | 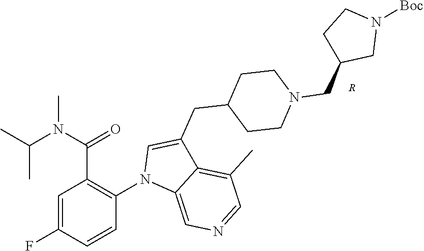 | Intermediate 28 & 149 |
| Compound 495 | 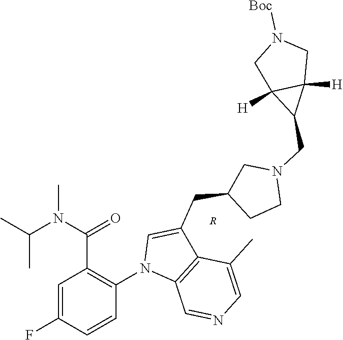 | Intermediate 202 & 250 |
| Compound 496 | 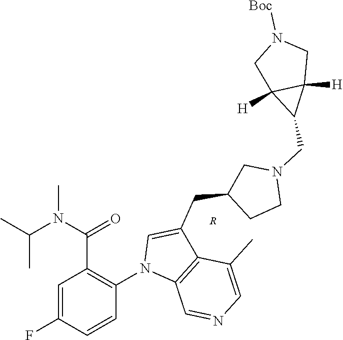 | Intermediate 202 & 287 |
| Compound 497 | 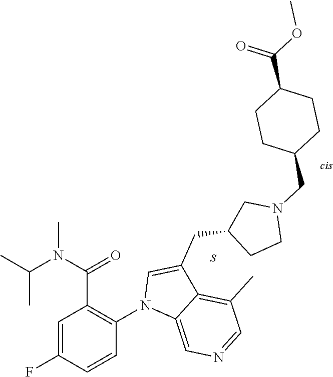 | Intermediate 222 & 225 |

### Preparation of Intermediate 179:

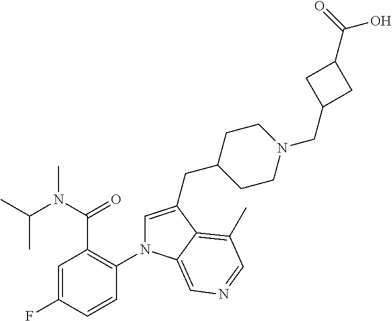 [SMILES: Cc1cncc2c1c(CC1CCN(CC3CC(C(=O)O)C3)CC1)cn2-c1ccc(F)cc1C(=O)N(C)C(C)C]

To a solution of Compound 501 (70 mg, 0.055 mmol) in methanol (3 mL) and tetrahydrofuran (3 mL) was added 2 M aqueous lithium hydroxide hydrate solution (0.14 mL, 0.274 mmol). The mixture was stirred at room temperature for 1 hour. The mixture was concentrated under reduced pressure. The residue was dissolved in water (10 mL) and washed with ethyl acetate (10 mL) for three times. The combined aqueous phase was acidified with 1 M aq. hydrogen chloride to pH=1 and the precipitate was filtered and dried in vacuo to give intermediate 179, which used directly in the next step.

The Following Intermediates were Synthesized by an Analogous Method as Described for Intermediate 179

| Int. No. | Structure | Starting Materials |
|---|---|---|
| Intermediate 209 | 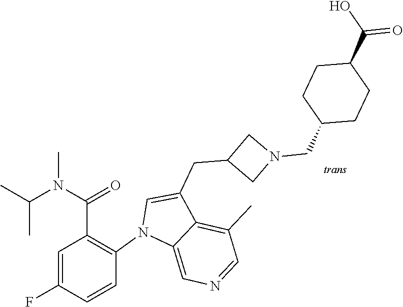 [SMILES: Cc1cncc2c1c(CC1CN(C[C@H]3CC[C@H](C(=O)O)CC3)C1)cn2-c1ccc(F)cc1C(=O)N(C)C(C)C] | Compound 498 |
| Intermediate 247 | 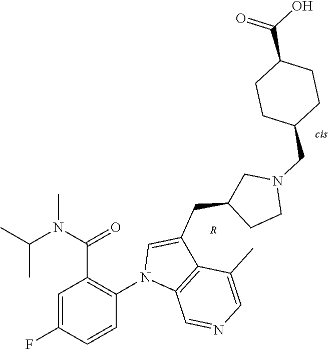 | Compound 153 |
| Intermediate 248 | 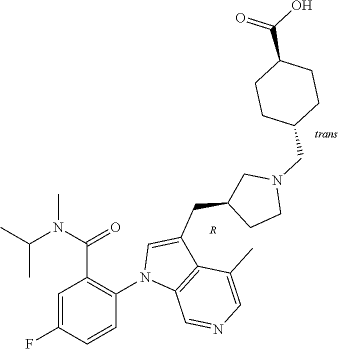 | Compound 154 |
| Intermediate 292 | 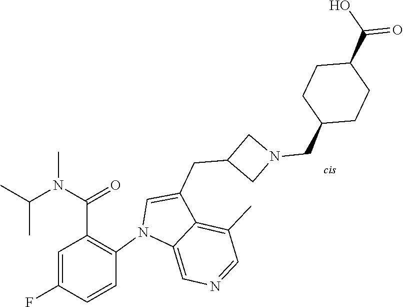 [SMILES: Cc1cncc2c1c(CC1CN(C[C@H]3CC[C@@H](C(=O)O)CC3)C1)cn2-c1ccc(F)cc1C(=O)N(C)C(C)C] | Compound 499 |
| Intermediate 316 | 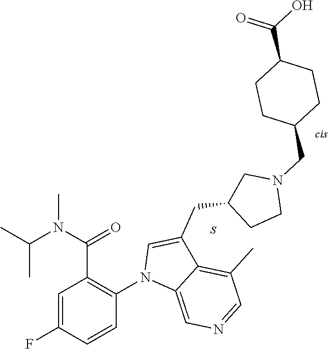 | Compound 497 |

### Preparation of Intermediate 180:

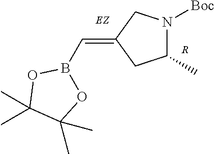

n-Butyllithium (2.5 M in hexane, 2.41 mL, 6.02 mmol) was added dropwise to a solution of 2,2,6,6-tetramethylpiperidine (0.88 g, 6.02 mmol) in tetrahydrofuran (11 mL) under N₂ at −40° C., after which the mixture was stirred at −40° C. for an extra 30 min. A solution of bis(4,4,5,5-tetramethyl-1,3,2-dioxaborolan-2-yl)methane (1.35 g, 5.02 mmol) in tetrahydrofuran (11 mL) was next added dropwise at −78° C. The resulting mixture was stirred at −78° C. for 30 min after which a solution of I-tert-butyl-2-methyl-4-oxopyrrolidine-1-carboxylate (1.0 g, 5.02 mmol) in tetrahydrofuran (11 mL) was added dropwise at −78° C. The mixture was allowed to warm to room temperature and stirred overnight. The mixture was quenched with saturated aq. ammonium chloride solution at 0° C. and stirred for an extra hour at 0° C. The precipitate was removed by filtration and the filtrate diluted with water and ethyl acetate. Phases were separated and the water layer was extracted with ethyl acetate. The combined organic layers were washed with brine, dried over Na₂SO₄, filtered and concentrated. The crude product was purified by silica column chromatography eluting with ethyl acetate in heptane from 0% to 10% to give intermediate 180 (958 mg, yield 59%).

The Following Intermediates were Synthesized by an Analogous Method as Described for Intermediate 180

| Int. No. | Structure | Starting Materials |
|---|---|---|
| Intermediate 181 | 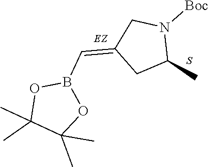 | (S)-tert-butyl-2-methyl-4- oxopyrrolidine-1-carboxylate |
| Intermediate 197 | 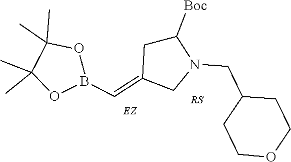 [SMILES: CC(C)(C)OC(=O)C1C/C(=C\B2OC(C)(C)C(C)(C)O2)CN1CC1CCOCC1] | Intermediate 196 |
| Intermediate 228 | 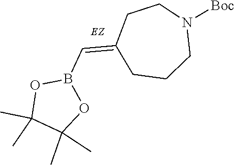 | t-butyl 4-oxoazepnae-1- carboxylate |
| Intermediate 301 | 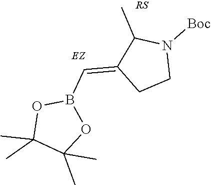 | t-butyl 2-methyl-3- oxopyrrolidine-1- carboxylate |

### Preparation of Intermediate 182

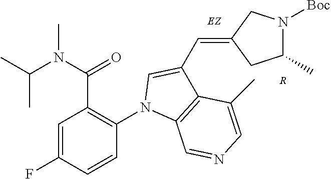

A reaction flask was consecutively charged with intermediate 6 (921 mg, 2.28 mmol), dioxane (7.1 mL), water (0.9 mL), intermediate 180 (958 mg, 2.96 mmol), cesium carbonate (1.49 g, 4.56 mmol) and 1,1′-bis(diphenylphosphino)ferrocene dichloropalladium(II) dichloromethane complex (186 mg, 0.23 mmol), degassed and refilled with nitrogen. The resulting mixture was stirred at 100° C. for 5 h. The reaction mixture was diluted with water and ethyl acetate. Phases were separated and the water layer was extracted with ethyl acetate. The combined organic layers were washed with brine, dried over MgSO₄, filtered and concentrated. The crude product was purified by silica gel column chromatography eluting with methanol in dichloromethane from 0% to 4% to give intermediate 182 (1.08 g, yield 87%) as a foam.

The Following Intermediates were Synthesized by an Analogous Method as Described for Intermediate 182

| Int. No. | Structure | Starting Materials |
|---|---|---|
| Intermediate 183 | 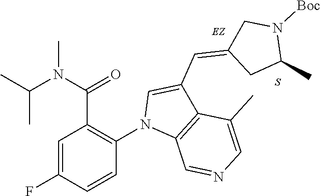 | Intermediate 6 & 181 |
| Intermediate 198 | 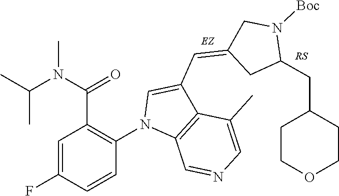 [SMILES: Cc1cncc2c1c(/C=C1\CC(CC3CCOCC3)N(C(=O)OC(C)(C)C)C1)cn2-c1ccc(F)cc1C(=O)N(C)C(C)C] | Intermediate 6 & 197 |
| Intermediate 229 | 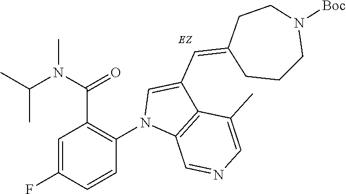 [SMILES: Cc1cncc2c1c(/C=C1\CCCN(C(=O)OC(C)(C)C)CC1)cn2-c1ccc(F)cc1C(=O)N(C)C(C)C] | Intermediate 6 & 228 |
| Intermediate 234 | 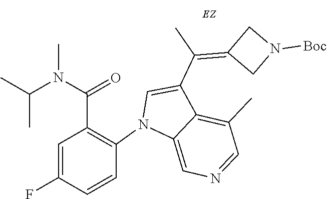 [SMILES: CC(=C1CN(C(=O)OC(C)(C)C)C1)c1cn(-c2ccc(F)cc2C(=O)N(C)C(C)C)c2cncc(C)c12] | Intermediate 6 & 233 |
| Intermediate 302 Intermediate 302a & Intermediate 302b | 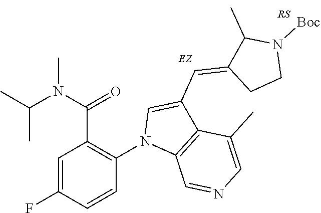 [SMILES: Cc1cncc2c1c(/C=C1\CCN(C(=O)OC(C)(C)C)C1C)cn2-c1ccc(F)cc1C(=O)N(C)C(C)C] | Intermediate 6 & 301 Intermediate 302 was separated by Prep SFC (Stationary phase: Chiralpak Diacel AD 20 × 250 mm, Mobile phase: CO₂, EtOH + 0.4 iPrNH₂). The first fraction was collected as intermediate 302a & the second fraction as intermediate 302b. |
| | 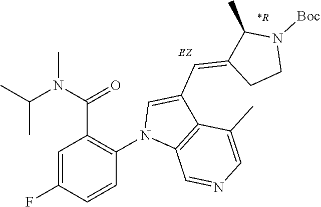 [SMILES: Cc1cncc2c1c(/C=C1\CCN(C(=O)OC(C)(C)C)[C@@H]1C)cn2-c1ccc(F)cc1C(=O)N(C)C(C)C] | |
| | 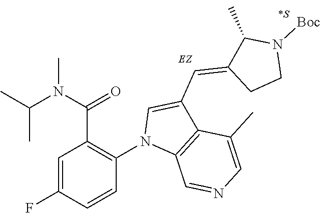 [SMILES: Cc1cncc2c1c(/C=C1\CCN(C(=O)OC(C)(C)C)[C@H]1C)cn2-c1ccc(F)cc1C(=O)N(C)C(C)C] | |

### Preparation of Intermediate 184 & 185:

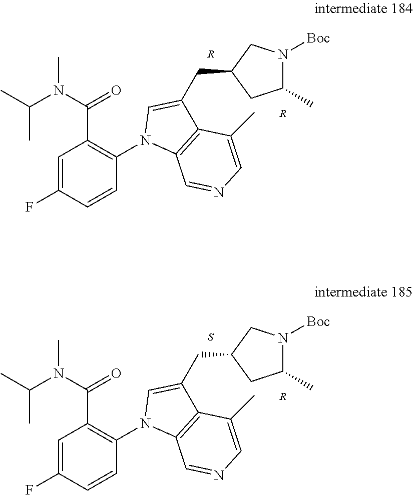

To a mixture of intermediate 182 (1.08 g, 2.07 mmol) in MeOH (100 mL) was added a catalytic amount of Pd/C (10% w:w) (221 mg, 0.207 mmol) and the solution was stirred under H₂ atmosphere overnight. Then, the mixture was filtered over Celite®, the Celite® washed with MeOH. The filtrate was concentrated under reduced pressure and the residue was purified by silica gel column chromatography eluting with methanol in dichloromethane from 0% to 5% to give the mixture of diastereomers (1030 mg, yield 91%) as a white foam, which were separated by chiral prep SFC (Stationary phase: Chiralpak Diacel AD 20×250 mm, Mobile phase: CO₂, iPrOH+0.4 iPrNH₂) to give intermediate 184 (192 mg, yield 18%) and intermediate 185 (551 mg, yield 51%). The absolute configuration was determined by NMR.

The Following Intermediates were Synthesized by an Analogous Method as Described for Intermediate 184 & 185

| Int. No. | Structure | Starting Materials |
|---|---|---|
| Intermediate 186 | 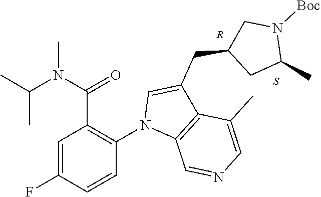 | Intermediate 183 |
| Intermediate 187 | 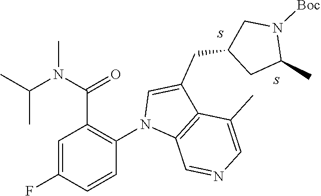 | Intermediate 183 |
| Intermediate 199 | 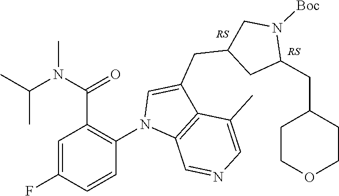 | Intermediate 198 |
| Intermediate 230 | 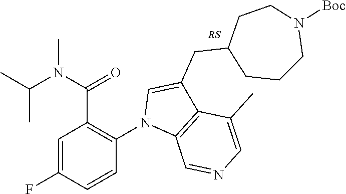 | Intermediate 229 |
| Intermediate 235 | 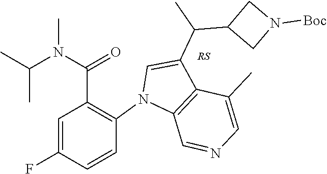 [SMILES: Cc1cncc2c1c(C(C)C1CN(C(=O)OC(C)(C)C)C1)cn2-c1ccc(F)cc1C(=O)N(C)C(C)C] | Intermediate 234 |
| Intermediate 303a & Intermediate 303b | 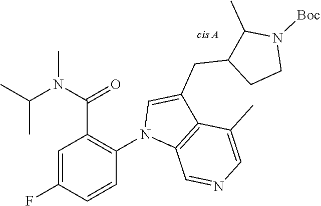 | Intermediate 302a The product was separated by Prep SFC (Stationary phase: Chiralpak Daicel IC 20 × 250 mm, Mobile phase: CO₂, EtOH + 0.4 iPrNH₂). The first fraction was collected as intermediate 303a & the second fraction as intermediate 303b. |
| | 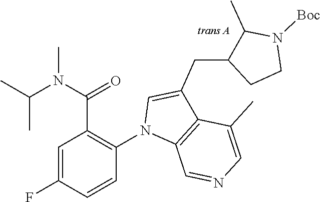 | |
| Intermediate 304a & Intermediate 304b | 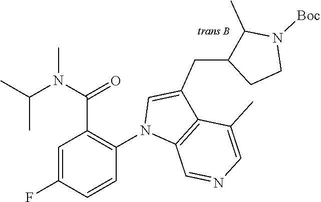 | Intermediate 302b The product was separated by Prep SFC (Stationary phase: Chiralpak Daicel IC 20 × 250 mm, Mobile phase: CO₂, EtOH + 0.4 iPrNH₂). The first fraction was collected as intermediate 304a & the second fraction as intermediate 304b. |
| | 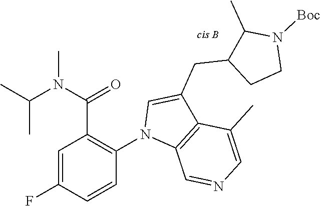 | |

### Preparation of Intermediate 192:

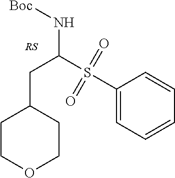 [SMILES: CC(C)(C)OC(=O)NC(CC1CCOCC1)S(=O)(=O)c1ccccc1]

A reaction flask was consecutively charged with tert-butyl carbamate (4.6 g, 39.0 mmol), sodium benzenesulfinate (9.6 g, 58.5 mmol), THF (16 mL), water (39 mL), 2-(tetrahydro-2H-pyran-4-yl)acetaldehyde (5.0 g, 39.0 mmol) and formic acid (10.3 mL, 273.1 mmol). The reaction mixture was stirred for 4 days at r.t. The precipitate was isolated by filtration, washed with water and dried in a vacuum oven at 50° C. to give intermediate 192 (10.9 g, yield 76%) as a white fluffy solid.

### Preparation of Intermediate 193:

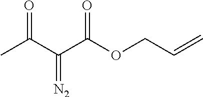

To a solution of allyl acetoacetate (5.0 g, 35.2 mmol), 4-acetamidobenzenesulfonyl azide (9.3 g, 38.7 mmol) in MeCN (176 mL) at 0° C. was added dropwise Et₃N (9.8 mmol, 70.3 mmol). The reaction mixture was allowed to warm to room temperature and stirred for 3 h. The solvent was removed under reduced pressure. The residue was suspended in diethyl ether and the solid (4-acetamidobenzenesulfonamide) was removed by filtration. The filtrate was concentrated under reduced pressure and the crude product purified by silica gel column chromatography eluting with ethyl acetate in heptane from 0% to 20% to give intermediate 193 (4.9 g, yield 83%) as a yellow oil.

### Preparation of Intermediate 194:

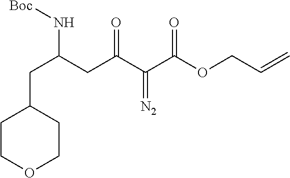

NaH (758 mg (60% dispersion in mineral oil), 18.9 mmol) was added portionwise to a solution of intermediate 192 (3185 mg, 18.9 mmol) in THF (80 mL) at r.t., after which stirring was continued for 20 min. Simultaneously Li-HMDS (18.9 mL, 1M in THF, 18.9 mmol) was added to a solution of intermediate 193 (3186 mg, 18.9 mmol) in THF (80 mL) at −78° C. The reaction mixture was stirred for 10 min at −78° C. after which the above reaction solution was added. The resulting reaction mixture was stirred for an additional 60 min after which it was quenched with 10 M acetic acid in THF. The mixture was warmed to room temperature and partitioned between EtOAc and water. The organic layer was separated, washed with water and brine, dried (MgSO₄), filtered and concentrated in vacuo. The residue was purified by silica gel column chromatography eluting with methanol in dichloromethane from 0% to 2% to give intermediate 194 (3.44 g, yield 46%).

### Preparation of Intermediate 195:

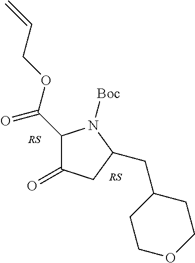 [SMILES: C=CCOC(=O)C1C(=O)CC(CC2CCOCC2)N1C(=O)OC(C)(C)C]

A mixture of intermediate 194 (500 mg, 1.26 mmol) and Rh₂(OAc)₄ (14 mg, 0.03 mmol) in DCM (30 mL) was stirred under a nitrogen atmosphere at rt for 2 hr. The reaction mixture was transferred as such to be purified by silica gel column chromatography eluting with methanol in dichloromethane from 0% to 2% to give intermediate 195 (262 mg, yield 57%) as a yellow oil.

### Preparation of Intermediate 196:

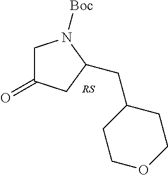 [SMILES: CC(C)(C)OC(=O)N1CC(=O)CC1CC1CCOCC1]

Pd(PPh3)₄ (8 mg, 0.007 mmol) and morpholine (750 mg, 8.61 mmol) were added to a solution of intermediate 195 (2110 mg, 5.74 mmol) in THF (136 mL) and stirred at room temperature overnight. The reaction mixture was concentrated to give a crude product which was purified by silica gel column chromatography eluting with methanol in dichloromethane from 0% to 2% to give intermediate 196 (1.2 g, yield 74%).

### Preparation of Intermediate 201—Method A:

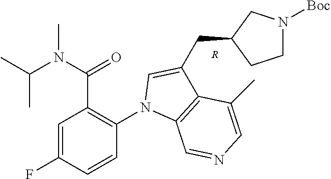

Into a 2 L 4-necked round-bottom flask were added THF (345 mL) and Zn (120.87 g, 1847.90 mmol, 5.00 equiv) at 30° C. under a nitrogen atmosphere. A solution of TMSCl (8.03 g, 73.91 mmol, 0.2 equiv) and 1-bromo-2-chloroethane (10.60 g, 73.91 mmol, 0.20 equiv) in THF (230 mL) were added into above round-bottom flask with a Lead Fluid-BT100F peristaltic pump (rate: 10 mL/min) under a nitrogen atmosphere. The resulting mixture was stirred for additional 40 min at 30° C. Next, a Lead Fluid-BT100F peristaltic pump was used to remove the solvent in above RBF quickly, and then fresh THF (575 mL) was re-charged under a nitrogen atmosphere. The mixture was heated to 60° C. Next, a solution of tert-butyl (3R)-3-(iodomethyl)pyrrolidine-1-carboxylate (115 g, 369.58 mmol, 1.00 equiv) in THF (575 mL) was added into above RBF with a Lead Fluid-BT100F peristaltic pump (rate: 15.0 mL/min) under a nitrogen atmosphere (temperature rises to 60-65° C.). The solution was stirred at 60° C. for an additional 1 h. The mixture was then cooled to 30° C. and allowed to stand for 1 h. The solution of {[(3R)-1-(tert-butoxycarbonyl)pyrrolidin-3-yl]methyl}(iodo)zinc was used directly in the next step. The concentration of the product was about 0.37 moL/L in THE Into a 2 L 4-necked round-bottom flask were added intermediate 6 (105 g, 259.71 mmol, 1.00 equiv) and THF (500 mL) at 30° C. under nitrogen atmosphere. To the stirred solution was added the 4th Generation RuPhos Pd precatalyst (5.65 g, 6.49 mmol, 0.025 equiv) under nitrogen atmosphere. Next, the solution of {[(3R)-1-(tert-butoxycarbonyl)pyrrolidin-3-yl]methyl}(iodo)zinc was added with a Lead Fluid-BT100F peristaltic pump into the 2 L 4-need RBF quickly under a nitrogen atmosphere (the excess zinc dust was not transferred). The resulting mixture was stirred for an additional 16 h at 50° C. The reaction was repeated 6 times in parallel. The reaction was quenched by the addition of aqueous sat. NH₄Cl solution (12 L). The aqueous layer was extracted with EtOAc (3×6 L), the organic layer was washed with water (2×3 L) and brine (1×3 L). The resulting mixture was dried with Na₂SO₄ and concentrated under reduced pressure. The crude product as a black oil (1100 g, crude) was used directly into the next step (preparation of intermediate 202)

### Alternatively, the Procedure Described Below can be Employed for the Preparation of Intermediate 201—Method B

A column (1.5 cm×15 cm) was stoppered with cotton wool and filled with granular zinc (20-mesh), 22 g. The column volume of the filled column was determined by measuring the time for THF to fill the column at 1 m/min flow rate. Column volume=4.3 mL. The zinc was activated by flowing a strong activating solution through the column at 0.5 mL/min for 10 mins. The strong activating solution consists of 1 mL TMSCl (0.67 M) & 0.75 mL chlorobromoethane (0.71 M) in 10 mL THF. After activation, the column was washed with dry THF: 10 mL, 1 ml/min. tert-butyl (R)-3-(iodomethyl)pyrrolidine-1-carboxylate (10 g, 37 mmol) was dissolved in THF (60 mL). The iodide solution was flowed through the activated zinc column at 50° C., flow rate 0.45 mL/min. After reaction: titration with iodine shows a concentration of 0.30 M.

Intermediate 6 (1.2 g, 2.4 mmol) was added with RuPhos Pd G4 (0.051 g, 0.06 mmol) in a sealed vial with a stirring bar in a glove box. Then, a solution of freshly made R-((1-(tert-butoxycarbonyl)-3-yl)methyl)zinc(II) iodide (12 mL, 0.3 M, 3.6 mmol) which was prepared by the above procedure was added. Next, the solution was heated to 50° C. under nitrogen atmosphere during 16 h. The solution was concentrated in vacuo and the residue redissolved in DCM. Next, water was added, followed by aq. Na₄EDTA solution (pH>10). The layers were separated and the water layer was extracted once more with DCM. Organic layers were combined, dried over Na₂SO₄, filtered and evaporated to dryness. The residue was purified by silica gel column chromatography eluting with methanol in dichloromethane from 000 to 10% to give intermediate 201 (1.4 g, 1.5 mmol (55% purity), 63& yield).

The Following Intermediates were Synthesized by an Analogous Method (Method B) as Described for Intermediate 201

| Int. No. | Structure | Starting Materials |
|---|---|---|
| Intermediate 212 | 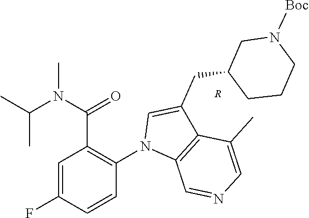 | Intermediate 6 & tert-butyl (S)- 3-(iodomethyl)piperidine-1- carboxylate |
| Intermediate 214 | 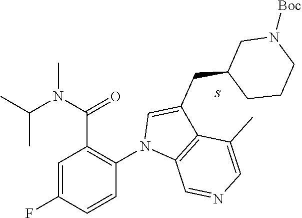 | Intermediate 6 & tert-butyl (R)- 3-(iodomethyl)piperidine-1- carboxylate |
| Intermediate 224 | 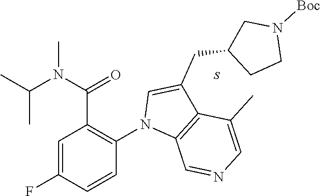 | Intermediate 6 & tert-butyl (S)- 3-(iodomethyl)pyrrolidine-1- carboxylate |
| Intermediate 318 | 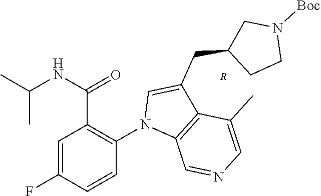 | Intermediate 317 & tert-butyl (R)-3-(bromomethyl)pyrrolidine- 1-carboxylate |
| Intermediate 322 | 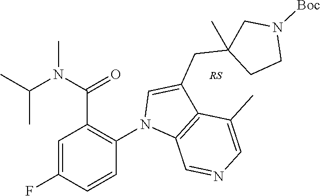 [SMILES: Cc1cncc2c1c(CC1(C)CCN(C(=O)OC(C)(C)C)C1)cn2-c1ccc(F)cc1C(=O)N(C)C(C)C] | intermediate 6 & tert-butyl 3- (bromomethyl)-3- methylpyrrolidine-1-carboxylate |
| Intermediate 337 | 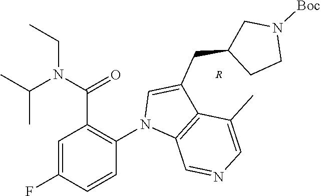 | Intermediate 7 & tert-butyl (R)- 3-(bromomethyl)pyrrolidine-1- carboxylate |
| Intermediate 341 | 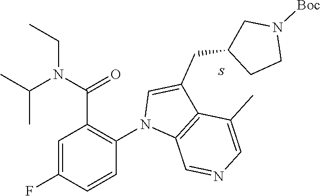 | Intermediate 7 & tert-butyl (S)- 3-(bromomethyl)pyrrolidine-1- carboxylate |
| Intermediate 346 | 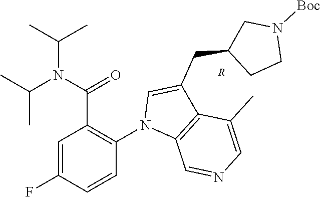 | Intermediate 345 & tert-butyl (R)-3-(bromomethyl)pyrrolidine- 1-carboxylate |
| Intermediate 353 | 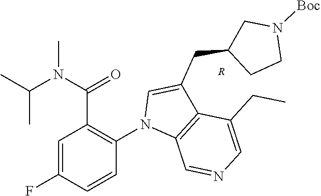 | Intermediate 352 & tert-butyl (R)-3-(bromomethyl)pyrrolidine- 1-carboxylate |
| Intermediate 357 | 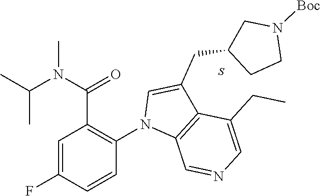 | Intermediate 352 & tert-butyl (S)-3-(bromomethyl)pyrrolidine- 1-carboxylate |
| Intermediate 367 | 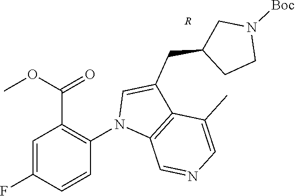 | Intermediate 361 & tert-butyl (R)-3-(iodomethyl)pyrrolidine- 1- carboxylate |

### Preparation of Intermediate 202:

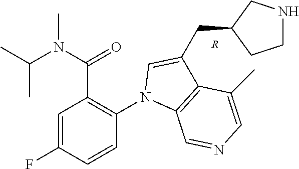

The mixture of intermediate 201 (17 g, 33.09 mmol) in dichloromethane (50 mL), was added the solution 24 mL of chlorine hydride (7 M in ethyl acetate). After stirring at r.t. for 5 h, the reaction mixture was concentrated, and the residue was diluted with DCM and basified with sodium hydroxide aqueous solution (1M) to pH˜ 10. The layers were separated and the aqueous layer was extracted with DCM three times and the combined organic layer was washed with brine (30 mL), dried over sodium sulfate, filtered and concentrated to afford intermediate 202 (13 g, 31.1 mmol, 94.2% yield) as a yellow solid, which was used in the next step without purification.

Alternatively, intermediate 202 can also be prepared as a 0.2TFA salt by using the following procedure:

Intermediate 201 (5.2 g, 6.95 mmol, 68% pure) is dissolved in DCM (44.5 mL) and TFA (5.3 mL) was added and stirred for 4 h at rt. The solution was concentrated in vacuo and coevaporated with toluene. Next, the mixture was washed with 1M NaOH and extracted four times with 10 DCM and EtOAc and Me-THF to obtain the combined organics which were then dried with anhydrous Na₂SO₄, filtered and concentrated in vacuo. The residue was purified via by silica gel column chromatography eluting with methanol (containing 7N NH₃) in dichloromethane from 0% to 10% to give intermediate 202 as a 0.2TFA salt.

Alternatively, intermediate 202 can also be prepared with the following procedure:

Into a 10 L 4-necked round-bottom flask were added 4N HCl in 1,4-dioxane (1.8 L). Then, crude intermediate 201 in THF (3 L) was added dropwise (calculated by 735 g intermediate 201, 1.82 mol, 1.0 equiv) at 0° C. The resulting mixture was stirred for an additional 2 h at 0° C. The resulting mixture was diluted with ethyl acetate (3 L) and water (3 L). The aqueous layer was washed with DCM (10×1 L). The pH of the aqueous layer was adjusted to pH 8 with saturated aqueous Na₂CO₃ solution and extracted with CH₂Cl₂ (4×2 L). The organic layers were dried with Na₂SO₄ and concentrated under vacuum to afford intermediate 202 (389 g, yield 53% over 2 steps) as a light yellow solid.

The Following Intermediate were Synthesized by an Analogous Method as Described for Intermediate 707

| Int. No. | Structure | Starting Materials |
|---|---|---|
| Intermediate 225 | 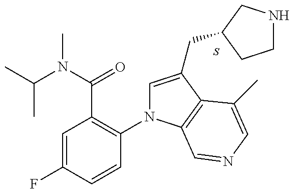 | Intermediate 224 |
| Intermediate 368 | 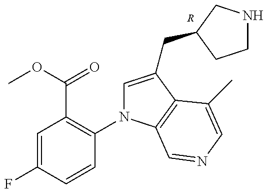 | Intermediate 367 |

### Preparation of Intermediate 203:

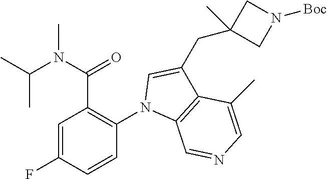 [SMILES: Cc1cncc2c1c(CC1(C)CN(C(=O)OC(C)(C)C)C1)cn2-c1ccc(F)cc1C(=O)N(C)C(C)C]

A stir bar, 4,4′-di-tert-butyl-2,2′-bipyridine (69.6 mg, 0.259 mmol), DME (40 mL), nickel(II) chloride ethylene glycol dimethyl ether complex (65.2 mg, 0.297 mmol) were added to 40 mL glass bottle, the mixture was purged with argon for 15 min, then intermediate 6 (1 g, 2.474 mmol), tert-butyl 3-(bromomethyl)-3-methylazetidine-1-carboxylate (1.3 g, 4.921 mmol), Ir[dF(CF₃)ppy]₂(dtbpy))PF₆ (282.6 mg, 0.252 mmol), sodium carbonate (782.6 mg, 7.384 mmol) and tris(trimethylsilyl)silane (1.3 mL, 4.214 mmol, 0.806 g/mL) were added to the mixture, the mixture was purged with argon for 15 min. The vial was sealed with parafilm and irradiated with blue light for 12 hours. The reaction mixture was diluted with dichloromethane (50 mL) and the saturated solution of sodium bicarbonate (50 mL) was added, the mixture was extracted with dichloromethane (40 mL×3). The combined organic layers were dried over anhydrous Na₂SO₄, filtered and concentrated under reduced pressure to give a residue which was purified by preparative-HPLC (Column: Boston Uni C18 40*150*5 um, Mobile Phase A: water, Mobile Phase B: acetonitrile, Flow rate: 60 mL/min, gradient condition from 30% B to 60% B). The pure fractions were collected and the solvent was evaporated under vacuum. The residue was partitioned between acetonitrile (2 mL) and water (10 mL). The mixture was lyophilized to dryness to give intermediate 203 (380 mg, 92.2% purity, 21.9% yield).

### Preparation of Compound 498:

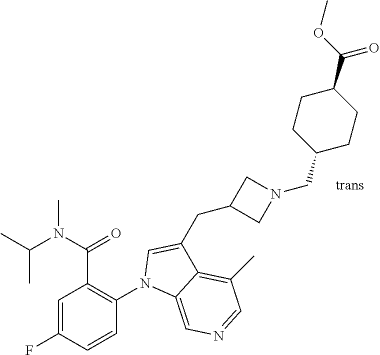 [SMILES: COC(=O)[C@H]1CC[C@H](CN2CC(Cc3cn(-c4ccc(F)cc4C(=O)N(C)C(C)C)c4cncc(C)c34)C2)CC1]

A stir bar, intermediate 25 (300 mg, 0.760 mmol), MeCN (3 mL), intermediate 207 (230 mg, 0.919 mmol), potassium carbonate (318 mg, 2.30 mmol) and potassium iodide (252 mg, 1.52 mmol) were added into a 8 mL glass. The reaction mixture was heated and stirred at 100° C. for 2 h under microwave irradiation. The reaction mixture was filtered through a pad of Celite®, the filter cake was washed with MeCN (5 mL×5). The combined filtrates were concentrated under reduced pressure to give the crude product which was purified by silica gel column chromatography eluting with methanol in dichloromethane from 0% to 9% to give Compound 498 (270 mg, 43.5% purity, 28.1% yield) as yellow solid.

### The Following Compound was Synthesized by an Analogous Method as Described for Compound 498

| Co No. | Structure | Starting Materials |
|---|---|---|
| Compound 499 | 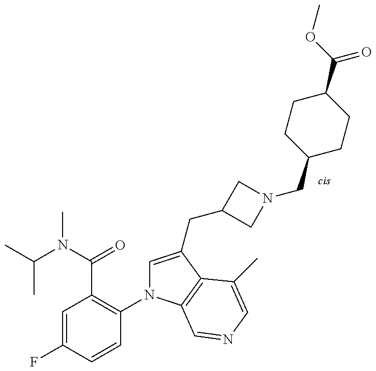 [SMILES: COC(=O)[C@H]1CC[C@@H](CN2CC(Cc3cn(-c4ccc(F)cc4C(=O)N(C)C(C)C)c4cncc(C)c34)C2)CC1] | Intermediate 25 & 290 |

### Preparation of Intermediate 211:

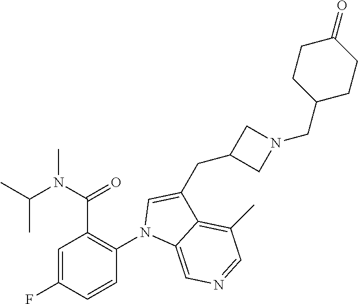 [SMILES: Cc1cncc2c1c(CC1CN(CC3CCC(=O)CC3)C1)cn2-c1ccc(F)cc1C(=O)N(C)C(C)C]

A mixture of Compound 92 (53 mg, 0.097 mmol) and iodine (2.5 mg, 0.01 mmol) in acetone (1.2 mL) was stirred at refluxing temperature (56° C.) for 10 min. The mixture was evaporated to dryness, and the crude was purified by silica gel column chromatography eluting with methanol (+1% NH₃ (7N) in methanol) in dichloromethane from 1% to 10% to give intermediate 211 (35 mg, yield 58.9%) as a white solid.

### The Following Intermediate was Synthesized by an Analogous Method as Described for Intermediate 211

| Int. No. | Structure | Starting Materials |
|---|---|---|
| Intermediate 264 | 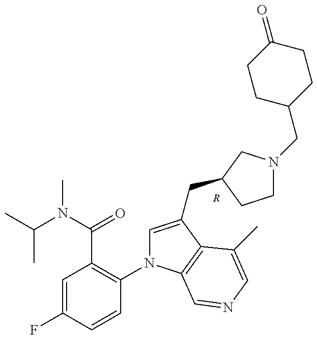 | Compound 407 |

### Preparation of Intermediate 216:

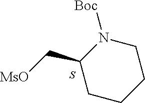

To a solution of (S)-tert-butyl 2-(hydroxymethyl)piperidine-1-carboxylate (400 mg, 1.86 mmol) in dichloromethane (8 mL) was added triethylamine (376 mg, 3.72 mmol) and methanesulfonyl chloride (277 mg, 2.42 mmol) at 0° C. The mixture was stirred at 0° C. for 60 minutes. The reaction was quenched with water and the mixture was diluted with dichloromethane, washed with 0.5M HCl (aq.), dried over Na₂SO₄, and concentrated to give intermediate 216 (385 mg, 17.5% purity from LCMS, 12.3% yield) as yellow oil which was used directly in the next step without further purification.

The Following Intermediates were Synthesized by an Analogous Method as Described for Intermediate 216

| Int. No. | Structure | Starting Materials |
|---|---|---|
| Intermediate 222 | 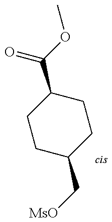 [SMILES: COC(=O)[C@H]1CC[C@@H](COS(C)(=O)=O)CC1] | cis-methyl 4- (hydroxymethyl)cyclo- hexanecarboxylate |
| Intermediate 223 | 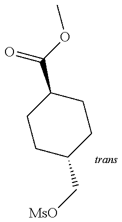 [SMILES: COC(=O)[C@H]1CC[C@H](COS(C)(=O)=O)CC1] | trans-methyl 4- (hydroxymethyl)cyclo- hexanecarboxylate |
| Intermediate 260 | 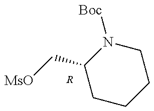 | (R)-tert-butyl 2- (hydroxymethyl)piperi- dine-1-carboxylate |

### Preparation of Intermediate 221:

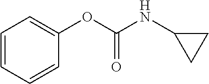 [SMILES: O=C(NC1CC1)Oc1ccccc1]

To a solution of cyclopropylamine (2 g, 33.3 mmol) in dichloromethane (25 mL) in an ice water bath was added triethylamine (10.1 g, 99.8 mmol) and phenyl chloroformate (5.2 g, 33.3 mmol) in five portions. The reaction mixture was stirred at room temperature for 2 hours. It was poured into water and extracted with dichloromethane (30 mL) twice. The organic layer was washed with brine (30 mL), dried over sodium sulfate, filtered and concentrated to afford the crude product, which was purified by silica gel column chromatography eluting with ethyl acetate in petroleum ether from 0% to 20% to give intermediate 221 (4.22 g, 98% purity, 70.1% yield) as a white solid.

### Preparation of Intermediate 233:

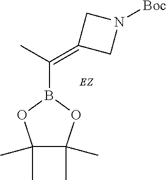 [SMILES: CC(B1OC(C)(C)C(C)(C)O1)=C1CN(C(=O)OC(C)(C)C)C1]

2,2,6,6-tetramethylpiperidine (3.90 g, 27.6 mmol) was dissolved in THF (50 mL) and cooled to −30° C. under N₂ atmosphere. n-BuLi (12.0 mL, 30.0 mmol, 2.5 M in n-Hexane) was added dropwise, and the reaction mixture was stirred at the same temperature for 30 minutes. Next, the reaction mixture was cooled to −78° C., and a solution of 2,2′-(ethane-1,1-diyl)bis(4,4,5,5-tetramethyl-1,3,2-dioxaborolane) (6.00 g, 21.3 mmol) in THF (30.0 mL) was added dropwise at −78° C. After stirring for 30 min, a solution of 1-boc-3-azetidinone (4.40 g, 25.7 mmol) in THF (40 mL) was added dropwise at −78° C. The reaction mixture was warmed to 25° C. slowly and stirred at 25° C. for 12 hours. The reaction mixture was cooled to 0° C. and quenched with aq. NH₄Cl solution (30 mL). After additional stirring for 10 minutes, the resulting mixture was concentrated under reduced pressure to remove THF, the residue was extracted with ethyl acetate (40 mL×2), and the organic layers were washed with brine (50 mL×1), dried over anhydrous Na₂SO₄, filtered, and concentrated under reduced pressure. The crude was purified by silica gel column chromatography eluting with ethyl acetate in petroleum ether from 0% to 6% to give intermediate 233 (4.00 g, 70% purity, 42.56% yield) as a colorless liquid.

### Preparation of Compound 500:

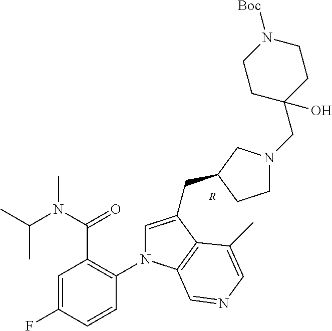

To a mixture of intermediate 202 (200 mg, 0.49 mmol) in EtOH (4 mL) and H₂O (0.4 mL) was added tert-butyl 1-oxa-6-azaspiro[2.5]octane-6-carboxylate (114.9 mg, 0.539 mmol) and TEA (49.6 mg, 0.49 mmol). The mixture was stirred at 25° C. for 12 hours. The mixture was neutralized with aqueous Na₂CO₃ (10 mL), poured into H₂O (20 mL) and extracted with DCM (3×20 mL). The combined organic layer was dried over anhydrous Na₂SO₄ which was purified by preparative-HPLC (Column: Welch Xtimate C18 150*30 mm*5 um, Mobile Phase A: water (NH₃H₂O+NH₄HCO₃), Mobile Phase B: acetonitrile, Flow rate: 35 mL/min, gradient condition from 47% B to 77% B). The pure fractions were collected and the solvent was evaporated under vacuum. The residue was partitioned between acetonitrile (2 mL) and water (10 mL). The mixture was lyophilized to dryness to give Compound 500 (100 mg, 30.94% yield) as a white powder.

### Preparation of Intermediate 258:

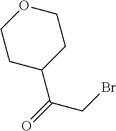

1-(tetrahydro-2H-pyran-4-yl)ethan-1-one (2.5 g, 19.5 mmol) was dissolved in MeOH (39.5 mL) and NBS (3471.6 mg, 19.5 mmol) was added and the solution was stirred for 3 hours at 50° C. The reaction mixture was concentrated in vacuo and redissolved in DCM and washed with water three times. The combined organics were dried and purified by silica gel column chromatography eluting with 30% ethyl acetate in heptane to give intermediate 258 (2.4 g, 59% yield).

### The Following Intermediate was Synthesized by an Analogous Method as Described for Intermediate 258

| Int. No. | Structure | Starting Materials |
|---|---|---|
| Intermediate 265 | 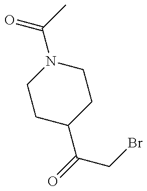 | 1-(4-acetylpiperidino)ethan- 1-one |

### Preparation of Intermediate 259:

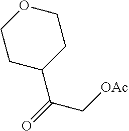 [SMILES: CC(=O)OCC(=O)C1CCOCC1]

Intermediate 258 (100 mg, 0.483 mmol) was dissolved in DMF (3.7 mL) and KOAc (142.19 mg, 1.449 mmol) was added and stirred for 4 hr at rt. The solution was extracted with EtOAc and washed with brine, and the combined organic layers were dried over Na₂SO₄ anhydrous, concentrated in vacuo and purified by silica gel column chromatography eluting with ethyl acetate in heptane from 0% to 100% to give intermediate 259 (65 mg, 72% yield) as an oil.

### _Preparation of Intermediate 266:

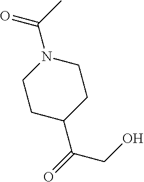

Intermediate 265 (0.50 g, 1.0 mmol, 50% purity) was dissolved in MeOH (12 mL), after which sodium formate (0.41 g, 6.0 mmol) was added. The resulting solution was heated at 55° C. overnight, after which it was evaporated to dryness. The residue was suspended in DCM and purified by silica gel column chromatography eluting with methanol in dichloromethane from 0% to 9% to give intermediate 266 (0.16 g, 0.78 mmol, 77% yield).

### Preparation of Intermediate 274:

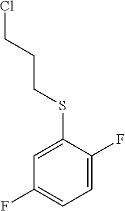 [SMILES: Fc1ccc(F)c(SCCCCl)c1]

1-bromo-3-chloropropane (0.37 mL, 3.76 mmol) was added to a stirred suspension of 2,5-difluorobenzenethiol (0.50 g, 3.42 mmol) and K₂CO₃ (0.61 g, 4.4 mmol) in anhydrous DMF (6.6 mL) and the mixture was left under stirring for overnight at rt. The mixture was diluted with water and extracted with EtOAc (×3). Reunited organic phases were washed with water (×2), brine (×1), dried over anhydrous sodium sulfate, filtered and evaporated to dryness to give intermediate 274 (951 mg, yield 93.6%) as a colorless oil. The desired product was used in the next step without further purification.

### Preparation of Intermediate 275:

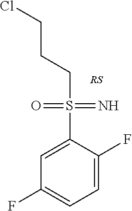 [SMILES: N=S(=O)(CCCCl)c1cc(F)ccc1F]

Iodobenzene diacetate (1.14 g, 3.54 mmol) was added to a solution of intermediate 274 (0.5 g, 1.684 mmol) and ammonium carbamate (0.276 g, 3.54 mmol) in MeOH (3.4 mL) at r.t. and the reaction mixture was stirred at r.t. overnight. The reaction mixture was diluted with water and extracted with DCM (×3). Reunited organic phases were dried over sodium sulfate, concentrated under reduced pressure and purified by silica gel column chromatography eluting with ethyl acetate in heptane from 10% to 100% to give intermediate 275 (281 mg, yield 65.7%) as a pale yellow oil.

### Preparation of Intermediate 276:

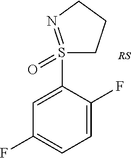 [SMILES: O=S1(c2cc(F)ccc2F)=NCCC1]

NH₃ (0.1% in H₂O, 4.3 mL) was added to intermediate 275 (288 mg, 1.13 mmol) in MeOH (0.5 mL) into a microwave vial, which was sealed and heated at 80° C. for 5 h. The solvent reaction was cooled down at rt, quenched with NaOH 1N, and extracted with EtOAc (×3). Reunited organic phases were washed with water, brine, dried over anhydrous sodium sulfate, filtered, and evaporated to dryness to afford intermediate 276 (213 mg, yield 86.4%) as a colorless oil.

### Preparation of Intermediate 299:

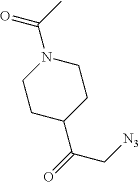

Intermediate 265 (0.50 g, 0.79 mmol) was dissolved in acetone (15 mL), after which, NaN₃ was added (0.16 g, 2.4 mmol). The mixture was stirred 50° C. for 1 h, after which the mixture was cooled to ambient temperature. Then, the mixture was filtered and evaporated to dryness. The residue was purified by silica gel column chromatography eluting with methanol in dichloromethane from 0% to 10% to give intermediate 299 (510 mg, 2.4 mmol).

### Preparation of Intermediate 300:

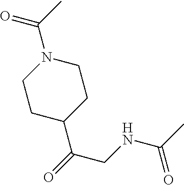 [SMILES: CC(=O)NCC(=O)C1CCN(C(C)=O)CC1]

Intermediate 299 (0.40 g, 1.95 mmol) was dissolved in THF (20 mL), after Ac₂O (0.18 mL, 2.0 mmol) and trimethylphosphine in THF (1M solution, 3.9 mL, 3.9 mmol) were added. The mixture was stirred at ambient temperature for 3 h. Then, MeOH was added and the mixture stirred at ambient temperature for ˜5 min. Next, the mixture was evaporated to dryness and the residue was purified by silica gel column chromatography eluting with methanol in dichloromethane from 0% to 8% to give intermediate 300 (0.24 g, yield: 54%).

### Preparation of Intermediate 324:

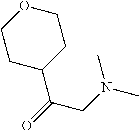

Intermediate 258 (320 mg, 1.54 mmol) in MeCN (3.2 mL) was treated sequentially with K₂CO₃ (640.7 mg, 4.6 mmol) and dimethylamine (2.3 mL, 2 M, 4.6 mmol). After stirring overnight at room temperature, the mixture was charged with aqueous 1N NaOH (2 mL), and the layers were separated. The aqueous layer was extracted with EtOAc (2×5 mL). The combined organic layers were dried over Na₂SO₄ anhydrous and concentrated under reduced pressure. The crude oil was further purified by silica gel column chromatography eluting with ethyl acetate (containing 25% EtOH) in heptane from 0% to 100% to give intermediate 324 as an oil (199 mg, 75% yield).

### Preparation of Intermediate 327:

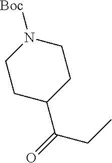 [SMILES: CCC(=O)C1CCN(C(=O)OC(C)(C)C)CC1]

To a solution of tert-butyl 4-(methoxy(methyl)carbamoyl)piperidine-1-carboxylate (2.5 g, 9.18 mmol) in tetrahydrofuran (30 ml) at 0° C. was added ethylmagnesium chloride (3.26 g, 36.7 mmol) and the resulting suspension was allowed to stir at room temperature for 4 hrs. After stirring at room temperature for 12 hours, the mixture was diluted with EtOAc, washed with sat. NH₄Cl and concentrated. The residue was purified by silica gel column chromatography eluting with ethyl acetate in petroleum ether from 20% to 100% to give intermediate 327 (3.1 g, yield: 83%).

### Preparation of Intermediate 332:

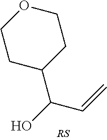

A solution of tetrahydro-2H-pyran-4-carbaldehyde (4 g, 33.29 mmol) in THF (20 mL) was dropwise vinylmagnesium bromide (67 mL) for 30 min at 0° C. The mixture was stirred at room temperature overnight. The mixture were quenched with 20 mL of NH₄Cl (aq) at 0° C. and extracted with EtOAc (20 mL*3). The combined organic layers were washed with brine, dried over Na₂SO₄, filtered and concentrated under reduced pressure to give the crude product, which was purified by silica gel column chromatography eluting with EtOAc in petroleum ether from 0% to 50% to give intermediate 332 (3 g, 57.0% yield) as a colourless oil.

### Preparation of Intermediate 333:

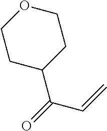

To a solution of intermediate 332 (3 g, 20.04 mmol) in dichloromethane (50 mL) was added Dess-Martin Periodinane (13.28 g, 30.06 mmol) at 0° C. After stirring at 20° C. for 5 h, the mixture was basified to pH 7-8 with saturated sodium bicarbonate aqueous solution and extracted with DCM (30 mL) for three times. The combined organic layers were washed with brine (30 mL), dried over Na₂SO₄ and filtered. The filtrate was concentrated under vacuum and purified by silica gel column chromatography eluting with 20% ethyl acetate in petroleum ether to give the intermediate 333 (1.5 g, 48.05% yield) as a yellow oil.

### Preparation of Intermediate 334:

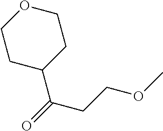

To a solution of intermediate 333 (500 mg, 3.21 mmol) in methanol (10 mL), was added sodium carbonate (aq.) (6.4 mL, 6.4 mmol) at rt. After stirring at rt for 18 h. The reaction mixture was quenched with H₂O (10 mL) and extracted with DCM. The combined organic phase was washed with brine, dried by Na₂SO₄, filtered and concentrated and purified by silica gel column chromatography eluting with 10% ethyl acetate in petroleum ether to give intermediate 334 (400 mg, 57.8% yield) as a yellow oil.

### Preparation of Intermediate 335:

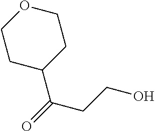 [SMILES: O=C(CCO)C1CCOCC1]

To a solution of intermediate 333 (1 g, 6.42 mmol) in H₂O/MeCN (20 mL/5 mL), was added chromium(II) chloride (204 mg, 1.28 mmol) at rt. After stirring at 80° C. for 18 h, the reaction mixture was quenched with H₂O (10 mL) and extracted with DCM. The combined organic phase was washed with brine, dried by Na₂SO₄, filtered and concentrated and purified by silica gel column chromatography eluting with ethyl acetate in petroleum ether from 50% to 100% to give intermediate 335 (800 mg, 63% yield) as a yellow oil.

### Preparation of Intermediate 361:

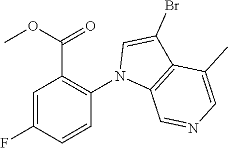 [SMILES: COC(=O)c1cc(F)ccc1-n1cc(Br)c2c(C)cncc21]

Intermediate 4 (3.3 g, 9.451 mmol) was dissolved in MeOH (38.2 mL) and cooled to 0° C. before thionyl chloride (13.7 mL, 189.0 mmol) was added dropwise. The solution was then heated to 70° C. for 2 hours. After cooling to ambient temperature, the solution was concentrated in vacuo and directly purified by silica gel column chromatography eluting with methanol (containing 7N NH₃) in dichloromethane from 0% to 10% to give intermediate 361 (3.7 g, 100% yield) as an oil.

### Preparation of Intermediate 366:

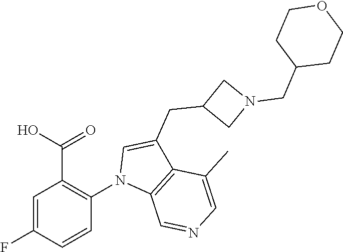 [SMILES: Cc1cncc2c1c(CC1CN(CC3CCOCC3)C1)cn2-c1ccc(F)cc1C(=O)O]

Compound 527 (1.8 g, 3.66 mmol, 92% pure) was dissolved in THF (29.8 mL) and water (6.62 mL) and LiOH (175.6 mg, 7.3 mmol) was added. The solution was stirred at r.t. for 16 hours until full conversion. The solution was concentrated till dryness, then co-evaporated with tolunene till dryness to obtain intermediate 366 as lithium salt with 1 eq LiOH as excess as a solid (1.7 g, 90% yield).

The Following Intermediates were Synthesized by an Analogous Method as Described for Intermediate 366

| Int. No. | Structure | Starting Materials |
|---|---|---|
| Intermediate 370 | 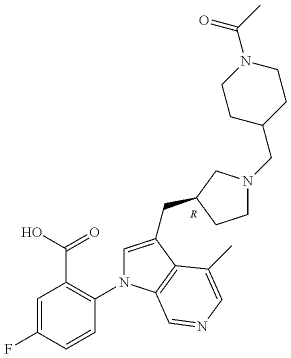 | Compound 528 |
| Intermediate 374 | 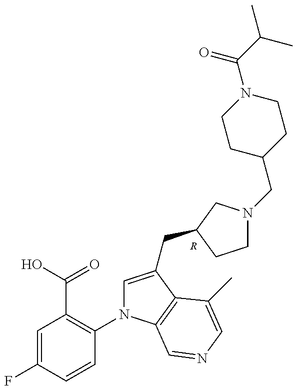 | Compound 516 |
| Intermediate 376 | 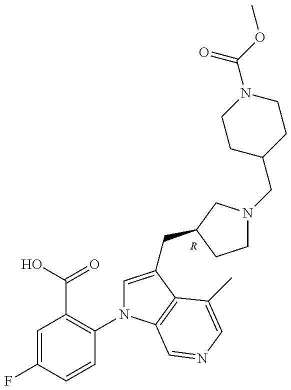 | Compound 517 |
| Intermediate 380 | 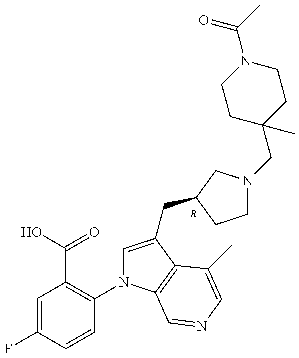 | Compound 518 |
| Intermediate 382 | 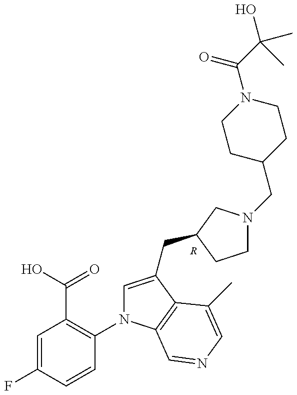 | Intermediate 381 |
| Intermediate 384 | 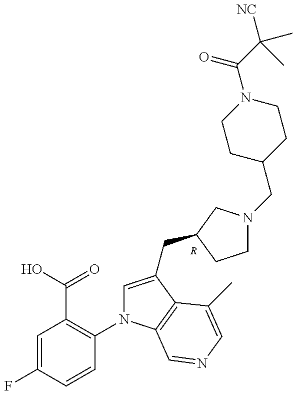 | Compound 521 |
| Intermediate 388 | 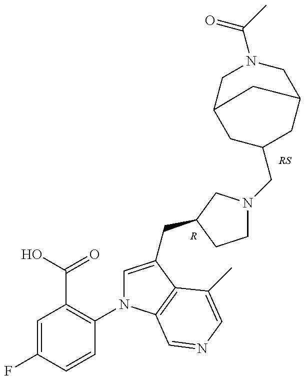 | Compound 519 |
| Intermediate 390 | 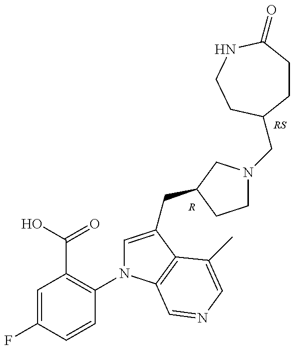 | Compound 533 |
| Intermediate 392 | 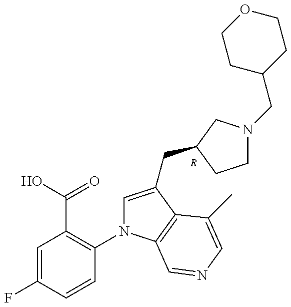 | Compound 534 |

### Preparation of Compound 516:

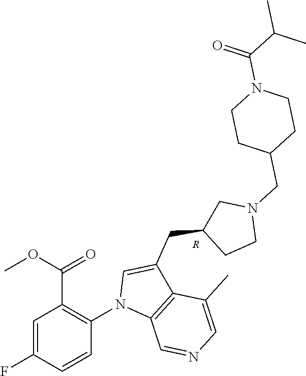

Compound 530 (641 mg, 1.2 mmol) was dissolved in MeCN (2.4 mL) and DIPEA (3.3 mL, 19.15 mmol) and isobutyryl chloride (1279 mg, 12 mmol) was added. The resulting mixture was stirred at rt for 16 h. Afterwards, the crude mixture was diluted with DCM and washed with water. The organic phase was dried over Na₂SO₄, filtered, and concentrated under reduced pressure. The crude mixture was purified using silica gel column chromatography eluting with ethyl acetate in heptane from 0% to 100% to afford Compound 516 (454 mg, 71% yield).

The Following Intermediates were Synthesized by an Analogous Method as Described for Compound 516

| Int. No. | Structure | Starting Materials |
|---|---|---|
| Compound 517 | 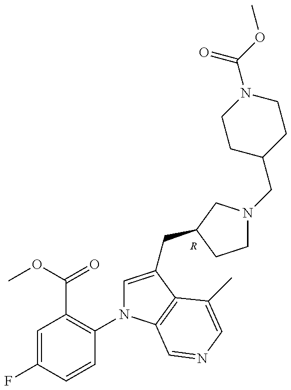 | Compound 350 & methyl chloroformate |
| Compound 518 | 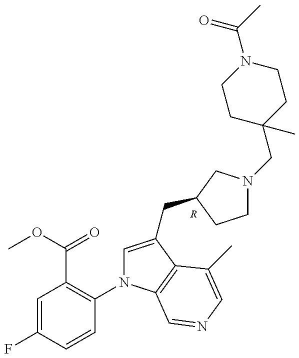 | Intermediate 378 & acetyl chloride |
| Compound 519 | 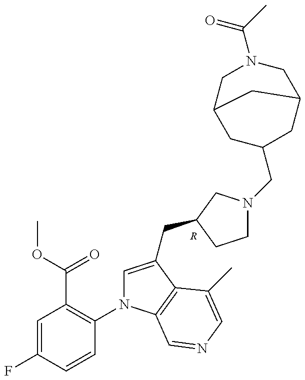 | Intermediate 386 & acetyl chloride |

### Preparation of Compound 520:

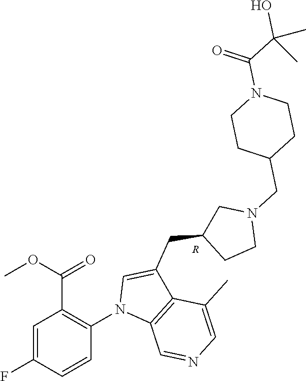

Compound 530 (870 mg, 0.945 mmol) is dissolved in DMF (7.3 mL) and DIPEA (0.97 mL, 5.67 mmol) and 2-hydroxy-2-methyl-propanoic acid (118.0 mg, 1.13 mmol) then HATU (538.9 mg, 1.4 mmol) are added and stirred for 2 hours at rt. The solution is extracted with EtOAc and washed three times with water (50 mL) and the combined organics are dried with MgSO₄ anhydrous, filtered and concentrated in vacuo. The crude was further purified by silica gel column chromatography eluting with methanol in dichloromethane from 0% to 10% to give Compound 520 (450 mg, 85% yield).

The Following Compounds were Synthesized by an Analogous Method as Described for Compound 520

| Co | | |
|---|---|---|
| No. | Structure | Starting Materials |
| Compound 521 | 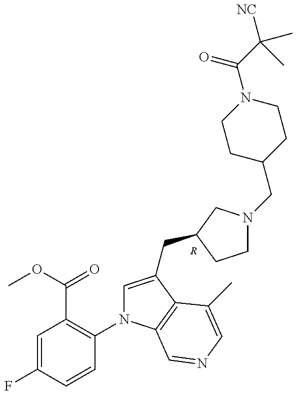 | Compound 530 & 2-cyano-2- methylpropanoic acid |

### Preparation of Intermediate 1:

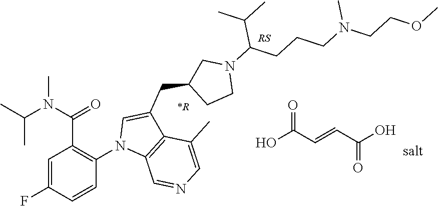

To a solution of intermediate 26 (300 mg, 98% purity, 0.72 mmol) in methanol (0.7 mL) were added intermediate 86 (457 mg, 95% purity, 2.16 mmol), sodium cyanoborohydride (136 mg, 2.16 mmol) and zinc chloride (294 mg, 2.16 mmol). The reaction mixture was heated up to 68° C. and stirred at this temperature overnight. After cooled down to r.t., the reaction mixture was concentrated and the residue was purified by prep. HPLC (column: SunFire C18 150*19 mm*5 um, Mobile Phase A: water (0.1% TFA), Mobile Phase B: acetonitrile, Flow rate: 15 mL/min, gradient condition from 13% B to 20% B)). The collected fraction was lyophilized and the residue was basified with sodium hydroxide aqueous solution (1M), extracted with dichloromethane (20 mL) twice. The combined organic layers were washed with brine (20 mL), dried over Na₂SO₄, filtered and concentrated to afford the free base of Compound 1 (120 mg, 99% purity, 25% yield) as a yellow solid. A solution of the free base (38 mg) and fumaric acid (12.6 mg) in water (5 mL) was freeze dried to give Compound 1 (50 mg, fumarate, 99.4% purity) as a yellow solid.

The Following Compounds were Synthesized by an Analogous Method as Described for Compound 1

| Compound No. | Structure | Starting Materials |
|---|---|---|
| Compound 2 | 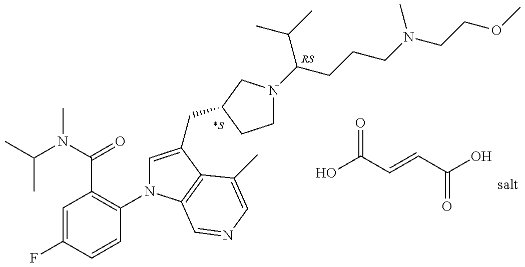 [SMILES: COCCN(C)CCCC(C(C)C)N1CC[C@H](Cc2cn(-c3ccc(F)cc3C(=O)N(C)C(C)C)c3cncc(C)c23)C1 ; O=C(O)/C=C/C(=O)O] | Intermediate 27 & 86 |
| Compound 2a | 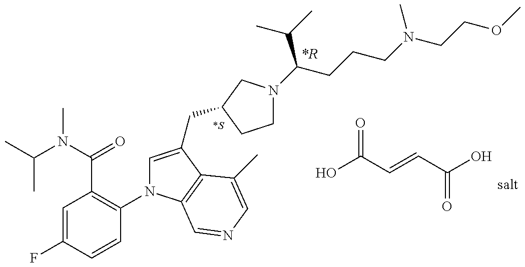 [SMILES: COCCN(C)CCC[C@H](C(C)C)N1CC[C@H](Cc2cn(-c3ccc(F)cc3C(=O)N(C)C(C)C)c3cncc(C)c23)C1 ; O=C(O)/C=C/C(=O)O] | Compound 2 (free base) was separated by chiral Prep. HPLC (separation condition: Column: Chiralpak AD-H, Column size: 0.46 cm I.D. × 15 cm L; Mobile Phase: Hexane:EtOH:DEA = 95:5:0.1, at 1 mL/min; Temp: 35° C.; Wavelength: 254 nm) and the first fraction was collected |
| Compound 3 | 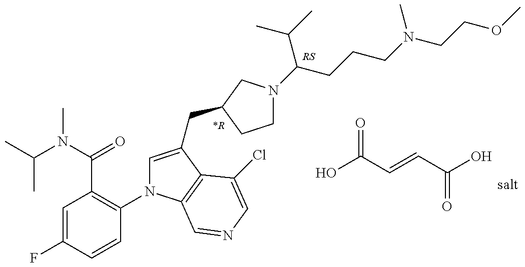 [SMILES: COCCN(C)CCCC(C(C)C)N1CC[C@@H](Cc2cn(-c3ccc(F)cc3C(=O)N(C)C(C)C)c3cncc(Cl)c23)C1 ; O=C(O)/C=C/C(=O)O] | Intermediate 31 & 86 |
| Compound 4 | 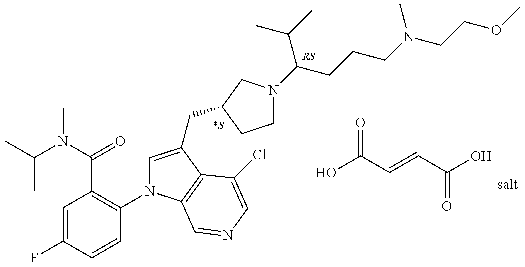 [SMILES: COCCN(C)CCCC(C(C)C)N1CC[C@H](Cc2cn(-c3ccc(F)cc3C(=O)N(C)C(C)C)c3cncc(Cl)c23)C1 ; O=C(O)/C=C/C(=O)O] | Intermediate 32 & 86 |
| Compound 4a | 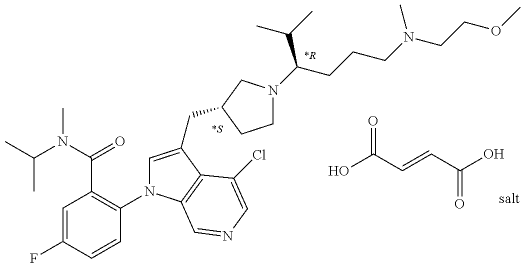 [SMILES: COCCN(C)CCC[C@H](C(C)C)N1CC[C@H](Cc2cn(-c3ccc(F)cc3C(=O)N(C)C(C)C)c3cncc(Cl)c23)C1 ; O=C(O)/C=C/C(=O)O] | Compound 4 (free base) was separated by SFC (column: DAICEL CHIRALPAK AD (2.5 cm I.D. × 25 cm L, 5 μm), eluent: supercritical CO₂ in Hexane/EtOH/DEA = 90/10/0.1 (V/V/V))) and the first fraction was collected |
| Compound 4b | 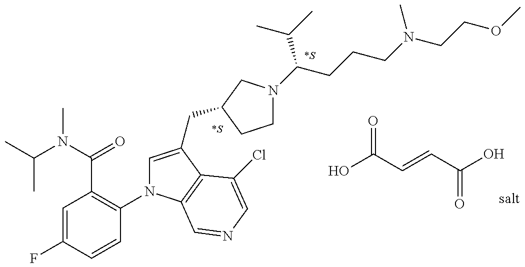 [SMILES: COCCN(C)CCC[C@@H](C(C)C)N1CC[C@H](Cc2cn(-c3ccc(F)cc3C(=O)N(C)C(C)C)c3cncc(Cl)c23)C1 ; O=C(O)/C=C/C(=O)O] | Compound 4 (free base) was separated by SFC (column: DAICEL CHIRALPAK AD (2.5 cm I.D. × 25 cm L, 5 μm), eluent: supercritical CO₂ in Hexane/EtOH/DEA = 90/10/0.1 (V/V/V))) and the second fraction was collected |
| Compound 6 | 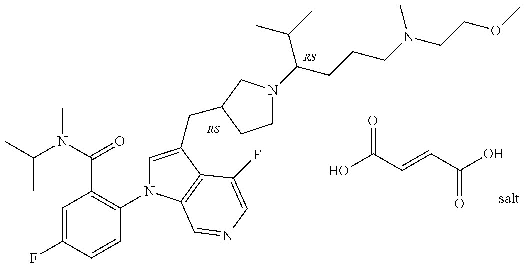 [SMILES: COCCN(C)CCCC(C(C)C)N1CCC(Cc2cn(-c3ccc(F)cc3C(=O)N(C)C(C)C)c3cncc(F)c23)C1 ; O=C(O)/C=C/C(=O)O] | 4-fluoro-1H-pyrrolo[2,3- c]pyridine |
| Compound 7 | 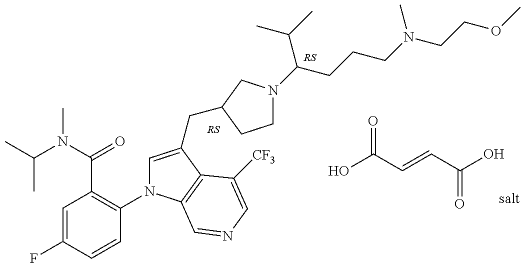 [SMILES: COCCN(C)CCCC(C(C)C)N1CCC(Cc2cn(-c3ccc(F)cc3C(=O)N(C)C(C)C)c3cncc(C(F)(F)F)c23)C1 ; O=C(O)/C=C/C(=O)O] | 4-(trifluoromethyl)-1H- pyrrolo[2,3-c]pyridine |

### Preparation of Compound 8:

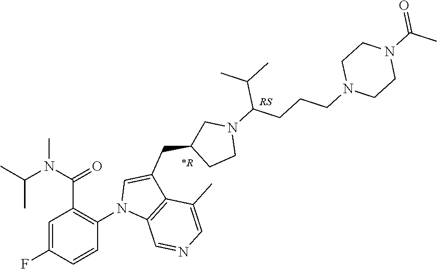

To a solution of intermediate 72 (100 mg, 98% purity, 0.188 mmol) in methanol (2 mL) were added 1-(piperazin-1-yl)ethanone (48.2 mg, 0.376 mmol) and acetic acid (0.05 mL). The reaction mixture was stirred at room temperature for 30 minutes. Then sodium cyanoborohydride (23.6 mg, 0.376 mmol) was added into the mixture. After stirring at r.t. for 2 hours, the reaction mixture was basified with saturated NaHCO₃ aqueous solution and extracted with dichloromethane (20 mL) twice. The combined organic layers were washed with brine (20 mL), dried over sodium sulfate, filtered and concentrated to afford the crude product, which was purified by Prep. HPLC (Column: SunFire C18 150*19 mm*5 um, Mobile Phase A: water (0.1% NH₄₀Ac), Mobile Phase B: acetonitrile, Flow rate: 15 mL/min, gradient condition from 10% B to 50% B) to give Compound 8 (100 mg, 99% purity, 83.1% yield) as a yellow gum.

The Following Compounds were Synthesized by an Analogous Method as Described for Compound 8

Alternatively, purification can also be performed using the following method: Prep. HPLC method (Column Welch Xtimate C18 150*25 mm*5 um, Mobile Phrase A: water (0.225% formic acid), mobile phase B: acetonitrile, Flow rate 25 mL/min, gradient condition from 1% B to 31% B).

| Compound No. | Structure | Starting Materials |
|---|---|---|
| Compound 8a | 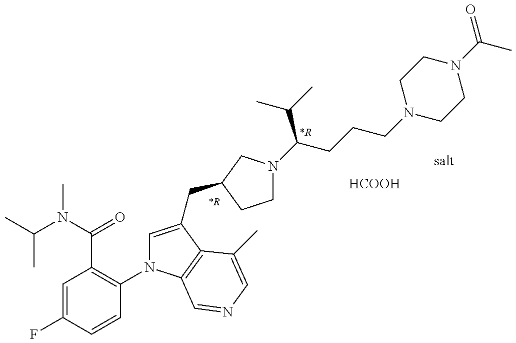 [SMILES: CC(=O)N1CCN(CCC[C@H](C(C)C)N2CC[C@@H](Cc3cn(-c4ccc(F)cc4C(=O)N(C)C(C)C)c4cncc(C)c34)C2)CC1 ; "HCOOH"] | Intermediate 72a & 1-(piperazin-1-yl)ethanone |
| Compound 8b | 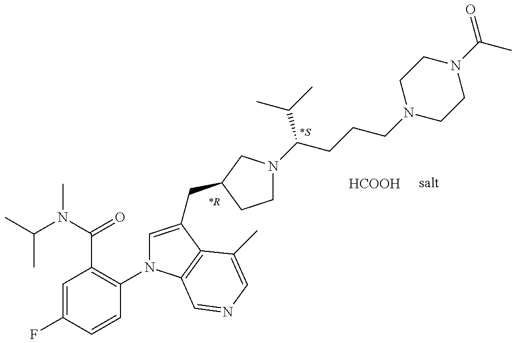 [SMILES: CC(=O)N1CCN(CCC[C@@H](C(C)C)N2CC[C@@H](Cc3cn(-c4ccc(F)cc4C(=O)N(C)C(C)C)c4cncc(C)c34)C2)CC1 ; "HCOOH"] | Intermediate 72b & 1-(piperazin-1-yl)ethanone |
| Compound 9 | 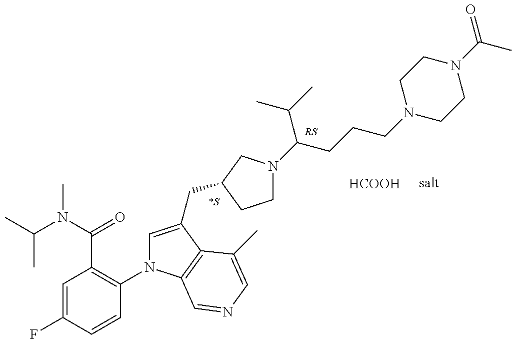 [SMILES: CC(=O)N1CCN(CCCC(C(C)C)N2CC[C@H](Cc3cn(-c4ccc(F)cc4C(=O)N(C)C(C)C)c4cncc(C)c34)C2)CC1 ; "HCOOH"] | Intermediate 73 & 1-(piperazin-1-yl)ethanone |
| Compound 9b | 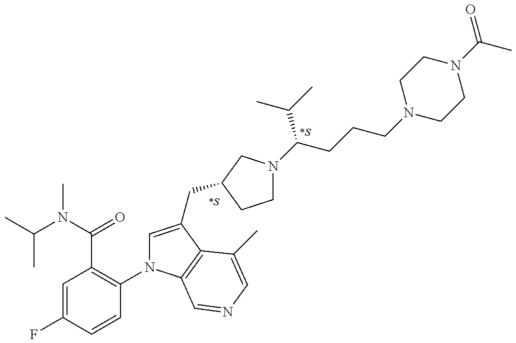 [SMILES: CC(=O)N1CCN(CCC[C@@H](C(C)C)N2CC[C@H](Cc3cn(-c4ccc(F)cc4C(=O)N(C)C(C)C)c4cncc(C)c34)C2)CC1] | Intermediate 73b & 1-(piperazin-1-yl)thanone |
| Compound 10 | 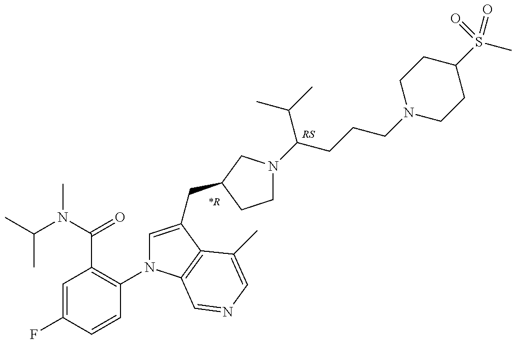 [SMILES: Cc1cncc2c1c(C[C@@H]1CCN(C(CCCN3CCC(S(C)(=O)=O)CC3)C(C)C)C1)cn2-c1ccc(F)cc1C(=O)N(C)C(C)C] | Intermediate 72 & 4-(methylsulfonyl)piperidine |
| Compound 11 | 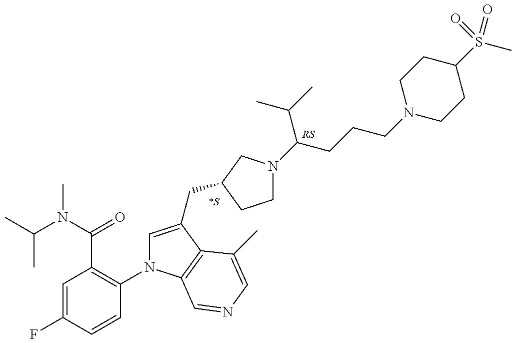 [SMILES: Cc1cncc2c1c(C[C@H]1CCN(C(CCCN3CCC(S(C)(=O)=O)CC3)C(C)C)C1)cn2-c1ccc(F)cc1C(=O)N(C)C(C)C] | Intermediate 73 & 4-(methylsulfonyl)piperidine |
| Compound 12 | 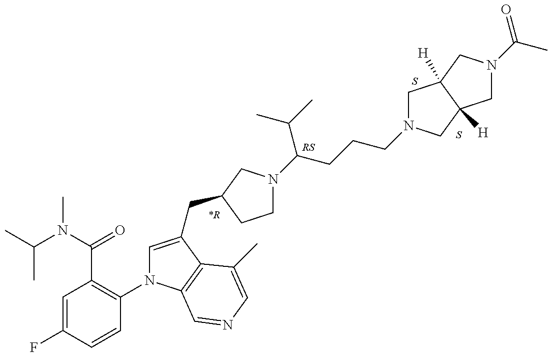 | Intermediate 72 & 89 |
| Compound 13 | 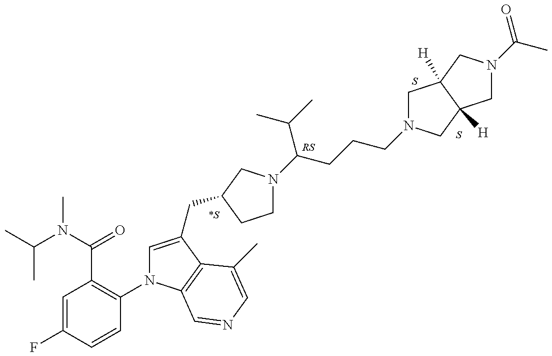 | Intermediate 73 & 89 |
| Compound 14 | 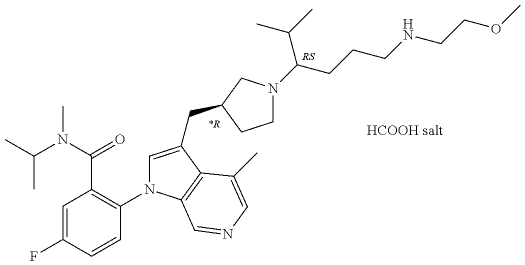 [SMILES: COCCNCCCC(C(C)C)N1CC[C@@H](Cc2cn(-c3ccc(F)cc3C(=O)N(C)C(C)C)c3cncc(C)c23)C1] | Intermediate 72 & 2-methoxyethanamine |
| Compound 15 | 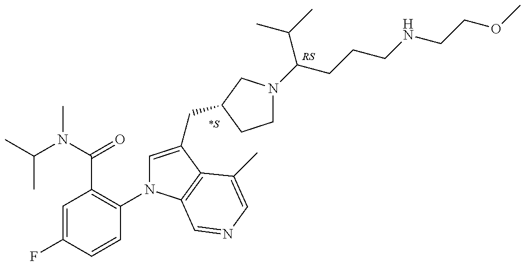 [SMILES: COCCNCCCC(C(C)C)N1CC[C@H](Cc2cn(-c3ccc(F)cc3C(=O)N(C)C(C)C)c3cncc(C)c23)C1] | Intermediate 73 & 2-methoxyethanamine |
| Compound 16 | 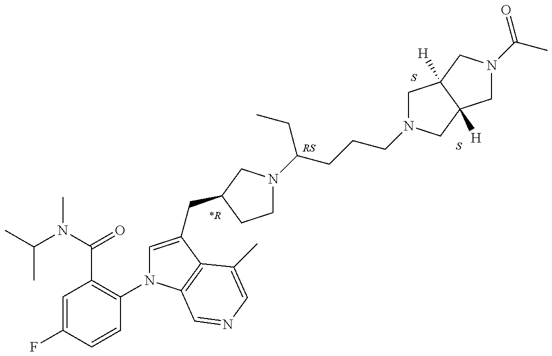 | Intermediate 74 & 89 |
| Compound 17 | 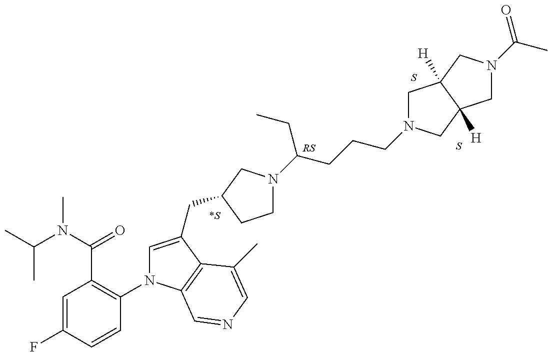 | Intermediate 75 & 89 |
| Compound 18a | 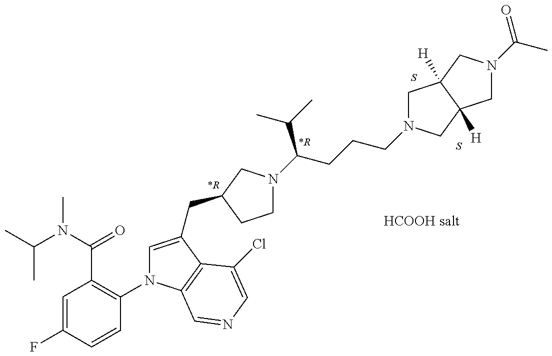 | Compound 371 & intermediate 89 |
| Compound 18b | 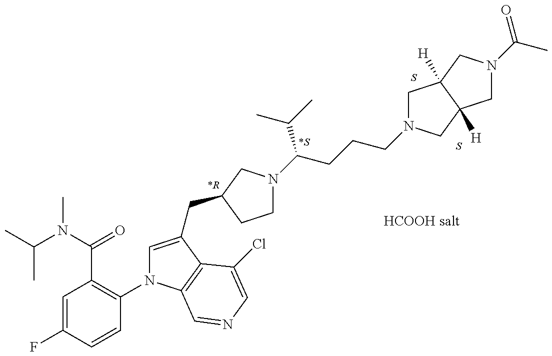 | Compound 372 & intermediate 89 |
| Compound 19a | 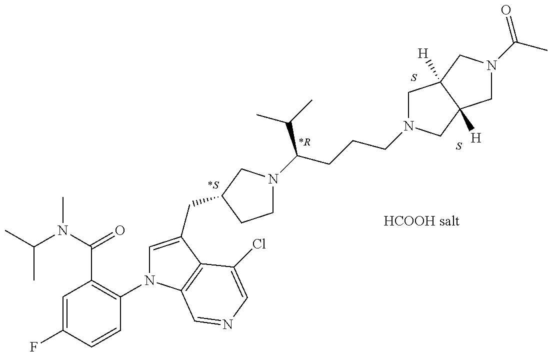 | Compound 374 & intermediate 89 |
| Compound 19b | 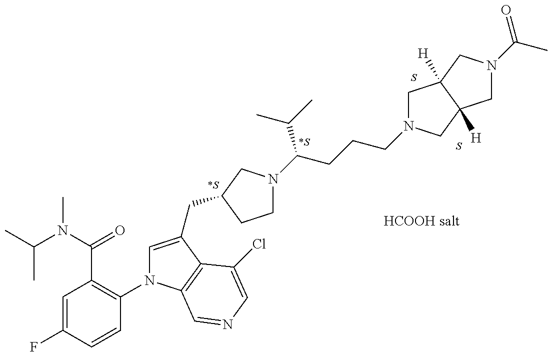 | Compound 75 & intermediate 89 |

### Preparation of Intermediate 9a:

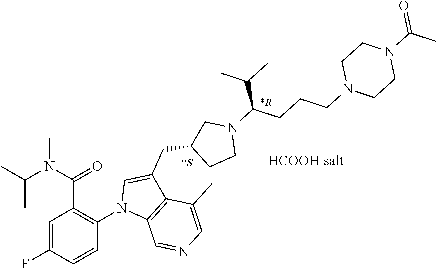

To a solution of intermediate 73a (300 mg, 0.576 mmol) and 1-(piperazin-1-yl)ethanone (148 mg, 1.16 mmol) in anhydrous methanol (5 mL) was added acetic acid (69.2 mg, 1.15 mmol). The reaction mixture was heated up to 45° C. and stirred at this temperature for 30 minutes before the addition of sodium cyanotrihydroborate (72.4 mg, 1.15 mmol). After stirring at 45° C. for another 12 hours, the reaction mixture was cooled down to room temperature, diluted with dichloromethane (40 mL), basified to pH=8 with the saturated solution of sodium bicarbonate (30 mL) and extracted with dichloromethane (20 mL×3). The combined organic layers were dried over Na₂SO₄, filtered and concentrated under reduced pressure to give a residue, which was purified by preparative-HPLC (Column: Boston Green ODS 150*30 mm*5 um, Mobile Phase A: water (0.225% FA), Mobile Phase B: acetonitrile, Flow rate: 35 mL/min, gradient condition from 1% B to 30% B). The pure fractions were collected, and the solvent was evaporated under vacuum. The residue was partitioned between acetonitrile (2 mL) and water (10 mL). The mixture was lyophilized to dryness to give Compound 9a (200 mg, 98.7% purity, 47.3% yield) as a yellow solid.

### Preparation of Compound 20:

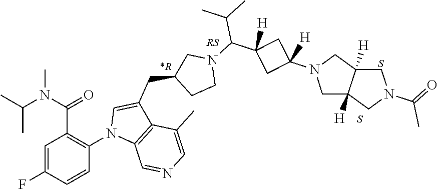

At 0° C., to a solution of intermediate 89 (56.2 mg, 90% purity, 0.19 mmol) in methanol (2 mL) was added sodium hydroxide aqueous solution (0.07 mL, 1M) until the pH to 9. Then, intermediate 64 (67 mg, 0.094 mmol) and sodium cyanoborohydride (11.8 mg, 0.189 mmol) were added into the mixture. After stirring at r.t. for 4 hours, the reaction mixture was concentrated and the residue was purified with Prep. HPLC (Column: Xbridge C18 150*19 mm*5 um, Mobile Phase A: water (0.1% TFA), Mobile Phase B: acetonitrile, Flow rate: 15 mL/min, gradient condition from 2% B to 30% B). The collected fraction was lyophilized and the residue was basified with sodium hydroxide aqueous solution (1M) and extracted with dichloromethane (20 mL) twice. The combined organic layers were washed with brine (20 mL), dried over Na₂SO₄, filtered and concentrated to afford the product which was lyophilized to give Compound 20 (22.1 mg, 97.3% purity, 34% yield) as a white solid.

The Following Compounds were Synthesized by an Analogous Method as Described for Compound 20

| Compound No. | Structure | Starting Materials |
|---|---|---|
| Compound 21 | 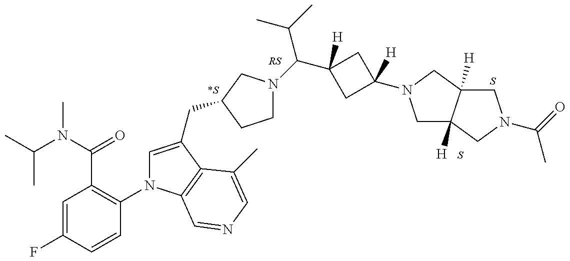 | Intermediate 65 & 89 |
| Compound 22 | 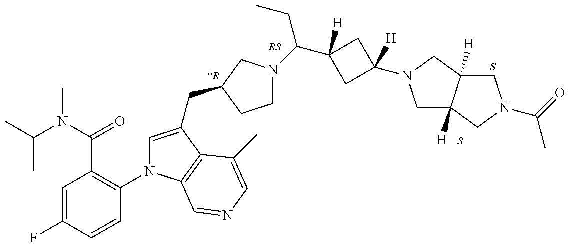 | Intermediate 66 & 89 |
| Compound 23 | 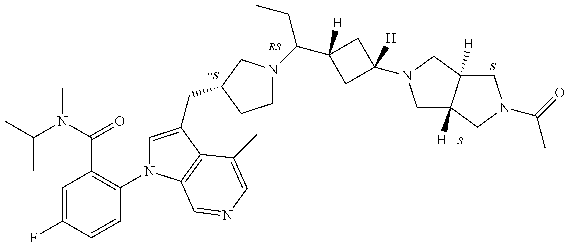 | Intermediate 67 & 89 |
| Compound 24 | 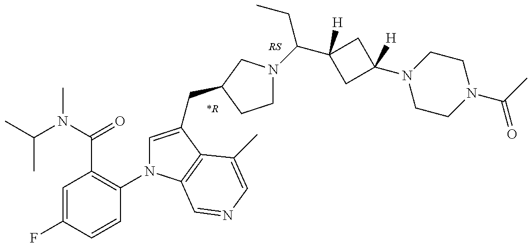 [SMILES: CCC([C@H]1C[C@@H](N2CCN(C(C)=O)CC2)C1)N1CC[C@@H](Cc2cn(-c3ccc(F)cc3C(=O)N(C)C(C)C)c3cncc(C)c23)C1] | Intermediate 66 & 1-(piperazin-1-yl)ethanone |
| Compound 25 | 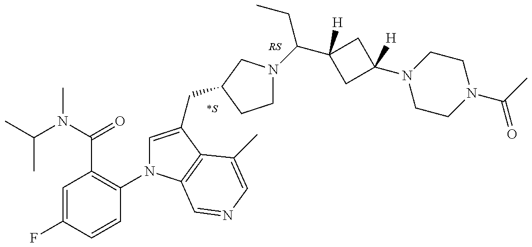 [SMILES: CCC([C@H]1C[C@@H](N2CCN(C(C)=O)CC2)C1)N1CC[C@H](Cc2cn(-c3ccc(F)cc3C(=O)N(C)C(C)C)c3cncc(C)c23)C1] | Intermediate 67 & 1-(piperazin-1-yl)ethanone |
| Compound 28 | 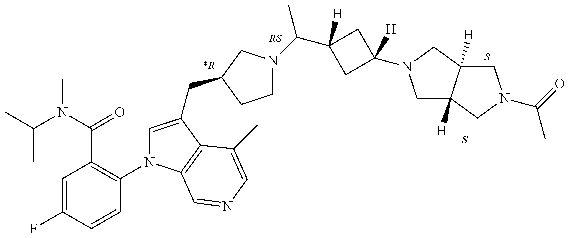 | Intermediate 68 & 89 |
| Compound 29 | 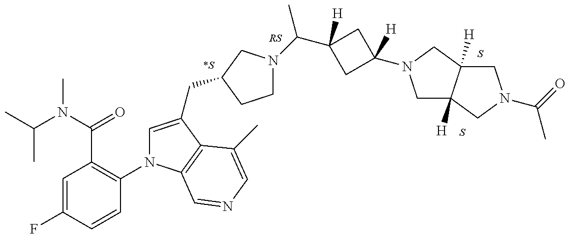 | Intermediate 69 & 89 |
| Compound 30 | 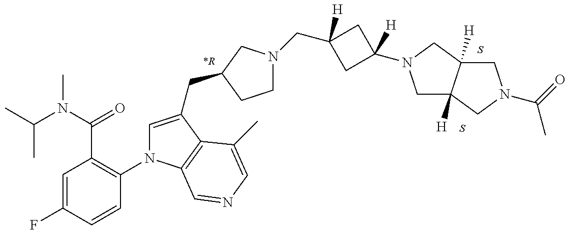 | Intermediate 70 & 89 |
| Compound 31 | 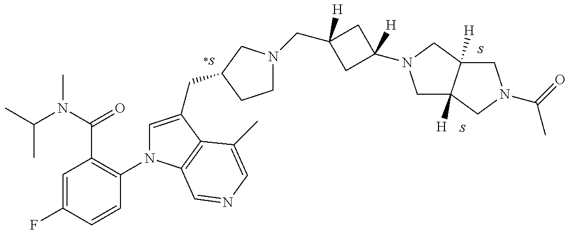 | Intermediate 71 & 89 |

### Preparation of Compound 26a & 26b:

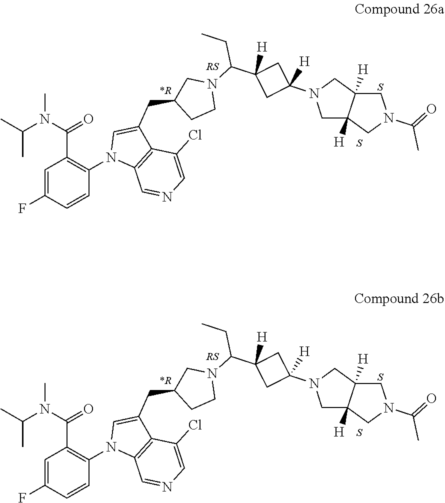

Triethylamine (113 mg, 1.12 mmol) was added to a solution of intermediate 89 (90 mg, 0.336 mmol) in dry dichloromethane (5 mL). Then intermediate 77 (120 mg, 0.223 mmol) was added. The reaction mixture was stirred at 25° C. for 30 minutes before the addition of sodium triacetoxyborohydride (95 mg, 0.448 mmol). After stirring at 25° C. for another 12 h, the reaction mixture was diluted with dichloromethane (50 mL) and saturated solution of sodium bicarbonate (50 mL). The mixture was extracted with dichloromethane (40 mL×3). The combined organic layers were dried over Na₂SO₄, filtered and concentrated under reduced pressure to give a residue, which was purified by preparative-HPLC (Column: Welch Xtimate C18 150*25 mm*5 um, Mobile Phase A: water (0.225% FA), Mobile Phase B: acetonitrile, Flow rate: 25 mL/min, gradient condition from 1% B to 28% B). The pure fractions were collected, and the solvent was evaporated under vacuum to give the mixture Compound 26a & 26b, which was further purified by preparative-HPLC (Column: Phenomenex Gemini-NX C18 75*30 mm*3 um, Mobile Phase A: water (0.04% NH₃H₂O+10 mM NH₄HCO₃), Mobile Phase B: acetonitrile, Flow rate: 25 mL/min, gradient condition from 35% B to 65% B). The pure fractions were collected, and the solvent was evaporated under vacuum. The residue was partitioned between acetonitrile (2 mL) and water (10 mL). The first faction was lyophilized to dryness to give Compound 26a (25 mg, 96.7% purity, 16.0% yield) as a white powder and the second fraction was lyophilized to dryness to give Compound 26b (20.0 mg, 95.3% purity, 12.6% yield) as a white powder.

The Following Compounds were Synthesized by an Analogous Method as Described for Compound 26a & 26b

| Compound No. | Structure | Starting Materials |
|---|---|---|
| Compound 27a | 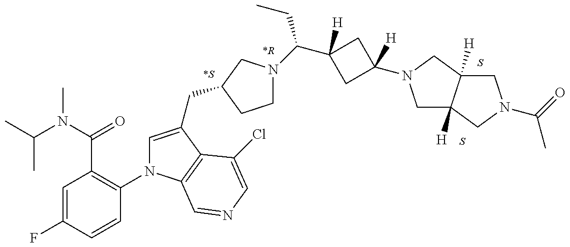 | Intermediate 78a & 89 |
| Compound 27b | 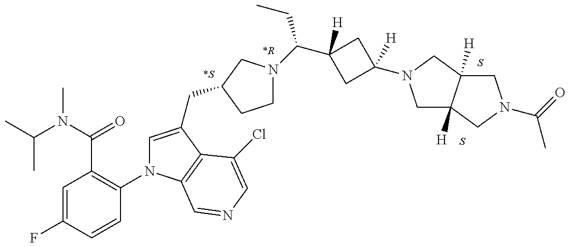 | |
| Compound 27c | 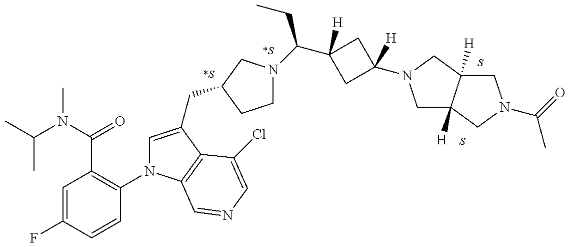 | Intermediate 78b & 89 |
| Compound 27d | 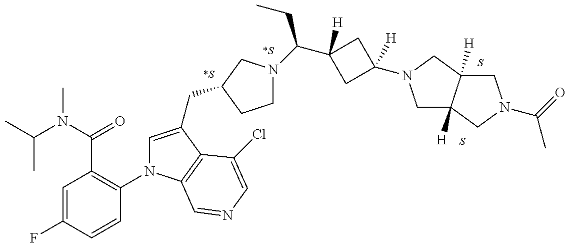 | |

### Preparation of Compound 32a:

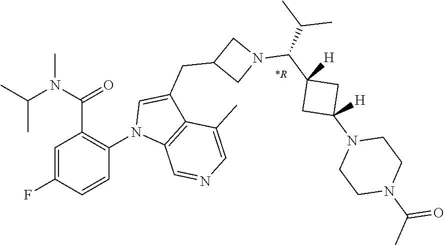 [SMILES: CC(=O)N1CCN([C@H]2C[C@@H]([C@@H](C(C)C)N3CC(Cc4cn(-c5ccc(F)cc5C(=O)N(C)C(C)C)c5cncc(C)c45)C3)C2)CC1]

To a solution of intermediate 62a (240 mg, 70% purity, 0.324 mmol) in methanol (5 mL) were added 1-(piperazin-1-yl)ethanone (83 mg, 0.648 mmol) and acetic acid (0.05 mL). The reaction mixture was stirred at room temperature for 30 minutes. Then sodium cyanoborohydride (40.7 mg, 0.648 mmol) was added into the mixture. After stirring at r.t. for 1 hr, the reaction mixture was basified with saturated NaHCO₃ aqueous solution and extracted with dichloromethane (20 mL) twice. The combined organic layers were washed with brine (20 mL), dried over sodium sulfate, filtered, and concentrated to afford the crude product, which was purified by prep. HPLC (Column: SunFire C18 150*19 mm*5 um, Mobile Phase A: water (0.1% TFA), Mobile Phase B: acetonitrile, Flow rate: 15 mL/min, gradient condition from 2% B to 40% B). The collected fraction was lyophilized and the residue was basified with sodium hydroxide aqueous solution (1 M) and extracted with dichloromethane (20 mL) twice. The combined organic layer was washed with brine (20 mL), dried over Na₂SO₄, filtered and lyophilized to afford Compound 32a (90 mg, 97.8% purity, 43% yield) as a white solid.

The Following Compounds were Synthesized by an Analogous Method as Described for Compound 32

| Compound No. | Structure | Starting Materials |
|---|---|---|
| Compound 32b | 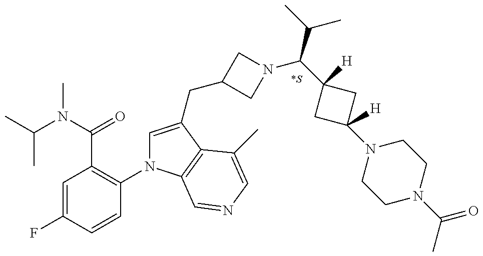 [SMILES: CC(=O)N1CCN([C@H]2C[C@@H]([C@H](C(C)C)N3CC(Cc4cn(-c5ccc(F)cc5C(=O)N(C)C(C)C)c5cncc(C)c45)C3)C2)CC1] | Intermediate 62b & 1- (piperazin-1-yl)ethanone |
| Compound 33c | 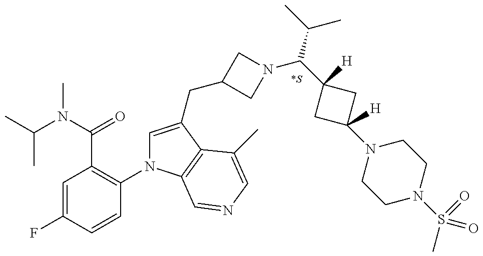 | Intermediate 62b & 4- (methylsulfonyl)piperidine |

### Preparation of Compound 33a & 33b:

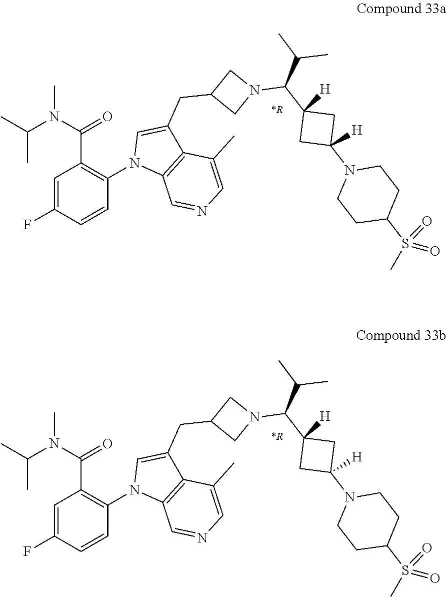 [SMILES: Cc1cncc2c1c(CC1CN([C@@H](C(C)C)[C@H]3C[C@@H](N4CCC(S(C)(=O)=O)CC4)C3)C1)cn2-c1ccc(F)cc1C(=O)N(C)C(C)C ; Cc1cncc2c1c(CC1CN([C@@H](C(C)C)[C@H]3C[C@H](N4CCC(S(C)(=O)=O)CC4)C3)C1)cn2-c1ccc(F)cc1C(=O)N(C)C(C)C]

To a solution of intermediate 62a (82 mg, 80% purity, 0.126 mmol) in methanol (5 mL) was added 4-(methylsulfonyl)piperidine (41.3 mg, 0.25 mmol) and acetic acid (0.05 mL). The reaction mixture was heated to 25° C. and stirred at this temperature for 30 minutes. Then sodium triacetoxyborohydride (15.9 mg, 0.25 mmol) was added and the reaction mixture was stirred at this temperature overnight. The reaction mixture was concentrated, and the residue was purified by Prep. HPLC (Column: SunFire C18 150*19 mm*5 um, Mobile Phase A: water (0.1% TFA), Mobile Phase B: acetonitrile, Flow rate: 15 mL/min, gradient condition from 10% B to 30% B). The collected fraction was lyophilized and the residue was basified with sodium hydroxide aqueous solution (1 M) and extracted with dichloromethane (20 mL) twice. The combined organic layers were washed with brine (20 mL), dried over Na₂SO₄, filtered and lyophilized to afford Compound 33a (40 mg, 99.8% purity, 47.4% yield) and Compound 33b (9 mg, 99.8% purity, 10.7% yield) as a white solid.

The Following Compounds were Synthesized by an Analogous Method as Described for Compound 33a & 33b

Alternatively, (additional) purification can also be performed using the following method Prep. HPLC method (Boston Green ODS 150*30 mm*5 um, Mobile Phase A: water (0.225% formic acid), Mobile Phase B: acetonitrile, Flow rate: 35 mE/min, gradient condition from 5% B to 35%).

| Compound No. | Structure | Starting Materials |
|---|---|---|
| Compound 34a | 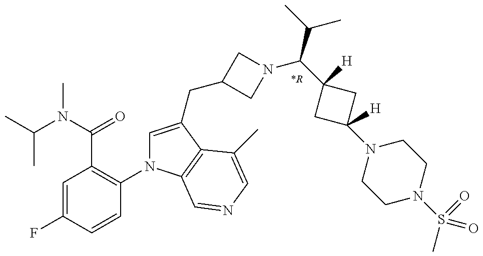 [SMILES: Cc1cncc2c1c(CC1CN([C@@H](C(C)C)[C@H]3C[C@@H](N4CCN(S(C)(=O)=O)CC4)C3)C1)cn2-c1ccc(F)cc1C(=O)N(C)C(C)C] | Intermediate 62a & 1- (methylsulfonyl)piperazine |
| Compound 34b | 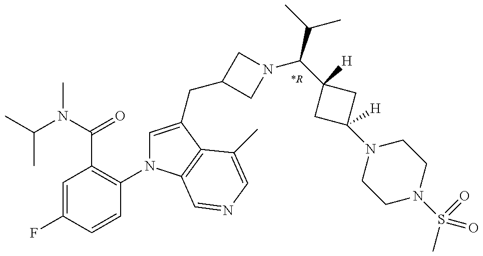 [SMILES: Cc1cncc2c1c(CC1CN([C@@H](C(C)C)[C@H]3C[C@H](N4CCN(S(C)(=O)=O)CC4)C3)C1)cn2-c1ccc(F)cc1C(=O)N(C)C(C)C] | |
| Compound 35a | 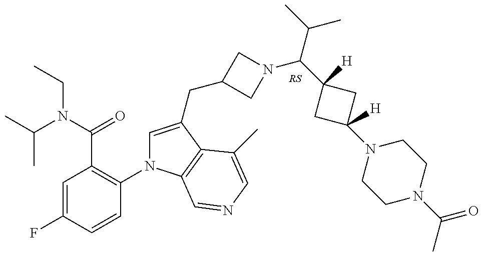 [SMILES: CCN(C(=O)c1cc(F)ccc1-n1cc(CC2CN(C(C(C)C)[C@H]3C[C@@H](N4CCN(C(C)=O)CC4)C3)C2)c2c(C)cncc21)C(C)C] | Intermediate 63 & 1-(piper- azin-1-yl)ethanone |
| Compound 35b | 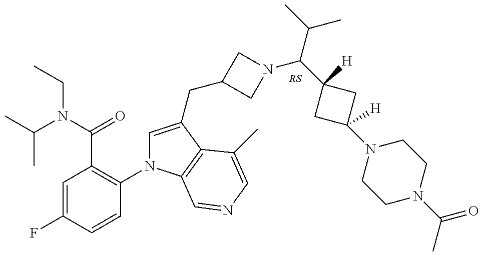 [SMILES: CCN(C(=O)c1cc(F)ccc1-n1cc(CC2CN(C(C(C)C)[C@H]3C[C@H](N4CCN(C(C)=O)CC4)C3)C2)c2c(C)cncc21)C(C)C] | |
| Compound 36a | 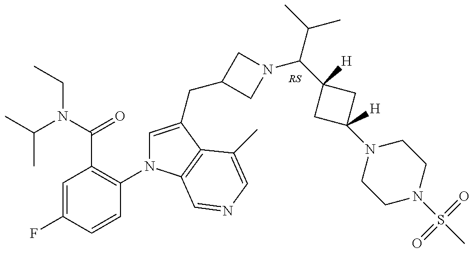 [SMILES: CCN(C(=O)c1cc(F)ccc1-n1cc(CC2CN(C(C(C)C)[C@H]3C[C@@H](N4CCN(S(C)(=O)=O)CC4)C3)C2)c2c(C)cncc21)C(C)C] | Intermediate 63 & 4- (methylsulfonyl)piperidine |
| Compound 36b | 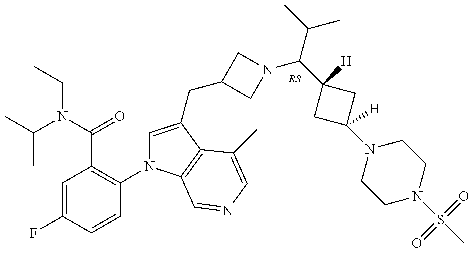 [SMILES: CCN(C(=O)c1cc(F)ccc1-n1cc(CC2CN(C(C(C)C)[C@H]3C[C@H](N4CCN(S(C)(=O)=O)CC4)C3)C2)c2c(C)cncc21)C(C)C] | |
| Compound 37a | 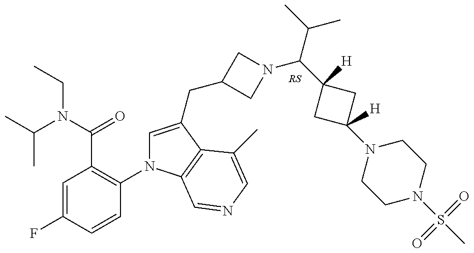 [SMILES: CCN(C(=O)c1cc(F)ccc1-n1cc(CC2CN(C(C(C)C)[C@H]3C[C@@H](N4CCN(S(C)(=O)=O)CC4)C3)C2)c2c(C)cncc21)C(C)C] | Intermediate 63 & 1- (methylsulfonyl)piperazine |
| Compound 37b | 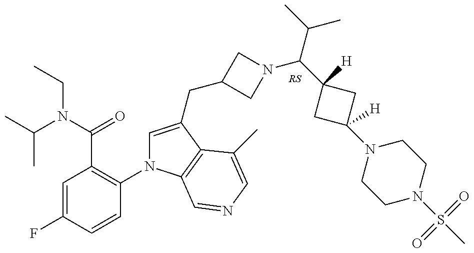 [SMILES: CCN(C(=O)c1cc(F)ccc1-n1cc(CC2CN(C(C(C)C)[C@H]3C[C@H](N4CCN(S(C)(=O)=O)CC4)C3)C2)c2c(C)cncc21)C(C)C] | |
| Compound 38a | 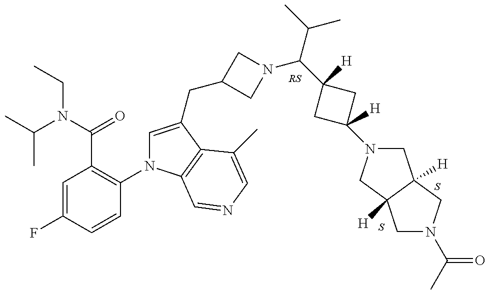 | Intermediate 63 & 89 |
| Compound 38b | 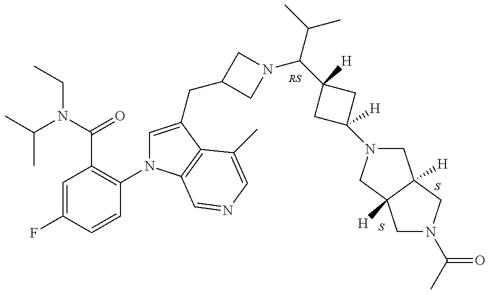 | |
| Compound 39a | 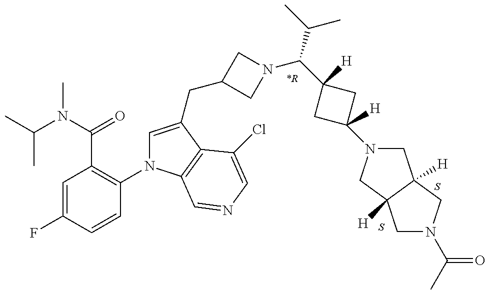 | Intermediate 76a & 89 |
| Compound 39b | 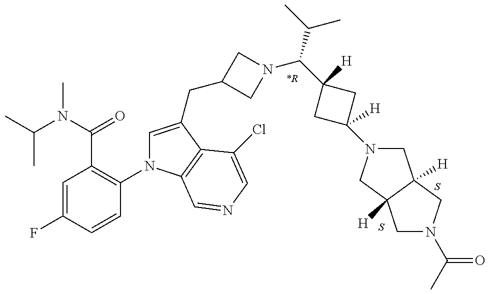 | |
| Compound 40a | 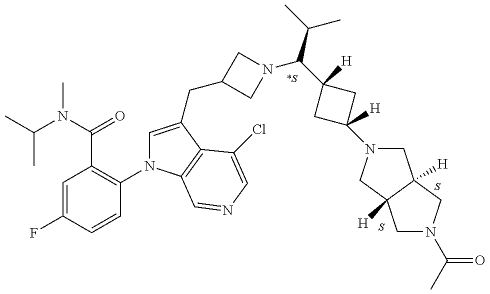 | Intermediate 76b & 89 |
| Compound 40b | 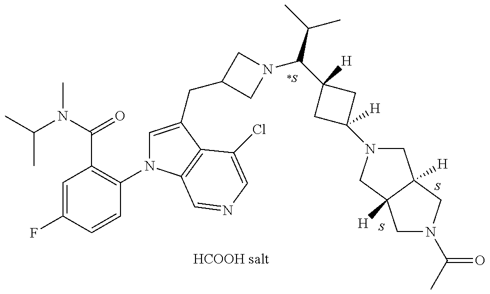 | |

### Preparation of Compound 41:

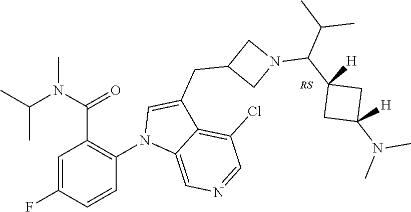 [SMILES: CC(C)C([C@H]1C[C@@H](N(C)C)C1)N1CC(Cc2cn(-c3ccc(F)cc3C(=O)N(C)C(C)C)c3cncc(Cl)c23)C1]

To a mixture of formaldehyde (194 mg, 6.46 mmol, 37% in H₂O), Compound 376 (420 mg, 0.647 mmol) in MeOH (4 mL) was added NaOAc (265 mg, 3.23 mmol). The mixture was stirred at 25° C. for 1 h. Then NaBH₃CN (81.6 mg, 1.30 mmol) was added to the mixture and the resulting mixture was stirred at 25° C. for 18 hours. The mixture was concentrated under reduced pressure to remove the solvent and the residue was diluted with ethyl acetate (10 mL), washed with saturated NaHCO₃ (10 mL), H₂O (10 mL) and brine (5 mL). The organic layer was dried over Na₂SO₄, filtered and concentrated under reduced pressure to give crude product, which was purified by preparative HPLC (Column: Boston Green ODS 150*30 mm*5 um, Mobile Phase A: water (0.225% FA), Mobile Phase B: acetonitrile, Flow rate: 35 mL/min, gradient condition from 10% B to 40% B). The pure fractions were collected, and the volatile solvent was evaporated under vacuum to give the residue, which was adjusted to pH=12 by NaOH (2 mol/L), then the mixture was extracted with ethyl acetate (20 mL). The organic phase was evaporated under vacuum to give the residue, which was lyophilized to afford the product (70 mg, purity 93.4%, yield 18%) as white solid.

### Preparation of Compound 42:

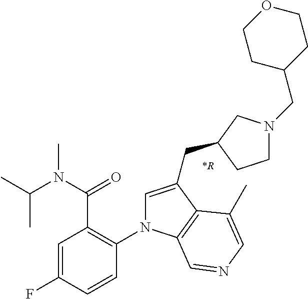 [SMILES: Cc1cncc2c1c(C[C@@H]1CCN(CC3CCOCC3)C1)cn2-c1ccc(F)cc1C(=O)N(C)C(C)C]

To a solution of intermediate 26 (80 mg, 0.192 mmol) in methanol (0.7 mL) was added tetrahydro-2H-pyran-4-carbaldehyde (69.2 mg, 0.576 mmol), NaBH₃CN (36.2 mg, 0.576 mmol) and acetic acid (0.05 mL). After stirring at r.t. overnight, the reaction mixture was concentrated and the residue was purified by prep. HPLC (Column: Xbridge C18 150*19 mm*5 um, Mobile Phase A: water (0.10% NH₄HCO₃), Mobile Phase B: acetonitrile, Flow rate: 15 mL/min, gradient condition from 30% B to 70% B). The collected fraction was lyophilized to give Compound 42 (40 mg, 99.5% purity, 40.9% yield) as a white solid.

### Preparation of Compound 43:

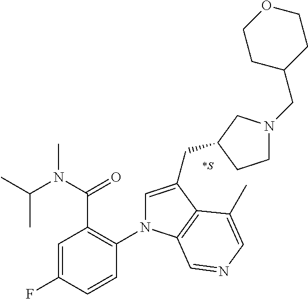 [SMILES: Cc1cncc2c1c(C[C@H]1CCN(CC3CCOCC3)C1)cn2-c1ccc(F)cc1C(=O)N(C)C(C)C]

To a mixture of intermediate 27 (90 mg, 0.22 mmol) and tetrahydro-2H-pyran-4-carbaldehyde (74 mg, 0.65 mmol) in methanol (2 mL) was added sodium cyanoborohydride (40 mg, 0.65 mmol). The reaction mixture was stirred at 20° C. overnight. The mixture was concentrated and purified by Prep. HPLC (Column: GiLSON-2 Xbridge C18 (5 μm 19*150 mm), Mobile phase A: water (0.1% ammonium bicarbonate), Mobile phase B: acetonitrile, UV: 214 nm, Flow rate: 15 mL/min, Gradient: 20% B to 60% B). The collected fraction was lyophilized to give Compound 43 (48 mg, 95% purity, 41% yield) as a white solid.

The Following Compounds were Synthesized by an Analogous Method as Described for Compound 43

In case reactions were performed with a ketone starting material, a typical procedure makes use of either 2 eq. acetic acid or 2 eq. of zinc(II)chloride (ZnCl₂), in the presence of 2 eq. sodium cyanoborohydride (NaCNBH₃), in methanol at 50° C. or 70° C. overnight.

| Compound No. | Structure | Starting Materials |
|---|---|---|
| Compound 44 | 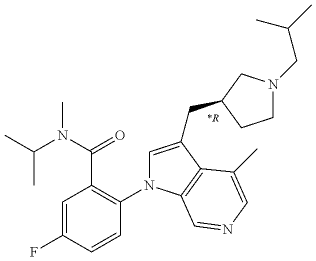 [SMILES: Cc1cncc2c1c(C[C@@H]1CCN(CC(C)C)C1)cn2-c1ccc(F)cc1C(=O)N(C)C(C)C] | Intermediate 26 & isobutyraldehyde |
| Compound 45 | 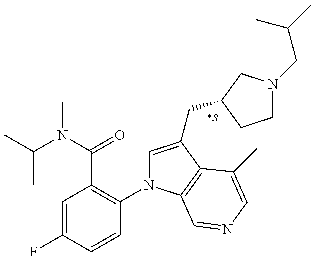 [SMILES: Cc1cncc2c1c(C[C@H]1CCN(CC(C)C)C1)cn2-c1ccc(F)cc1C(=O)N(C)C(C)C] | Intermediate 27 & isobutyraldehyde |
| Compound 46 | 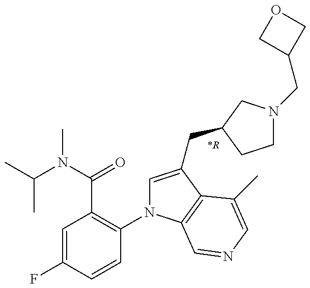 [SMILES: Cc1cncc2c1c(C[C@@H]1CCN(CC3COC3)C1)cn2-c1ccc(F)cc1C(=O)N(C)C(C)C] | Intermediate 26 & oxetane-3- carbaldehyde |
| Compound 47 | 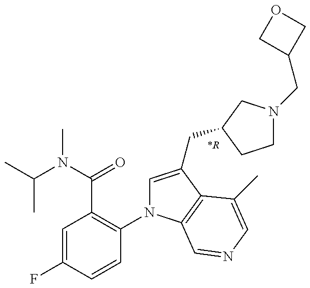 [SMILES: Cc1cncc2c1c(C[C@H]1CCN(CC3COC3)C1)cn2-c1ccc(F)cc1C(=O)N(C)C(C)C] | Intermediate 27 & oxetane-3- carbaldehyde |
| Compound 48 | 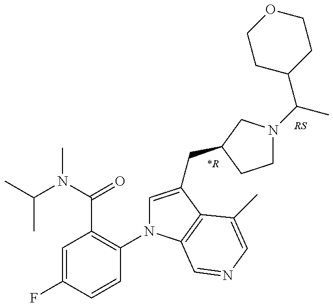 [SMILES: Cc1cncc2c1c(C[C@@H]1CCN(C(C)C3CCOCC3)C1)cn2-c1ccc(F)cc1C(=O)N(C)C(C)C] | Intermediate 26 & 1-(tetrahydro- 2H-pyran-4-yl)ethan-1-one |
| Compound 49 | 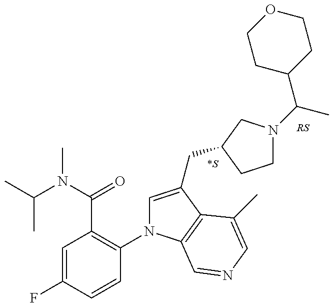 [SMILES: Cc1cncc2c1c(C[C@H]1CCN(C(C)C3CCOCC3)C1)cn2-c1ccc(F)cc1C(=O)N(C)C(C)C] | Intermediate 27 & 1-(tetrahydro- 2H-pyran-4-yl)ethan-1-one |
| Compound 49a & Compound 49b | 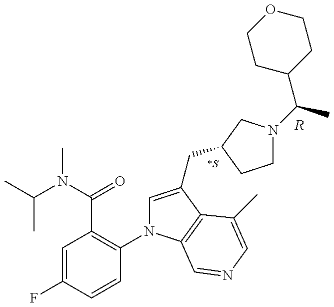 | Compound 49 was separated by supercritical fluid chromatography (separation condition: Phenomenex- Cellulose-2 (250 mm*30mm, 10um)); Mobile phase: A: Supercritical CO₂, B: 0.1% NH₃H₂O EtOH, A:B = 60:40 at 80 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was colleted as Compound 49a and the second fraction was Compound 49b |
| | 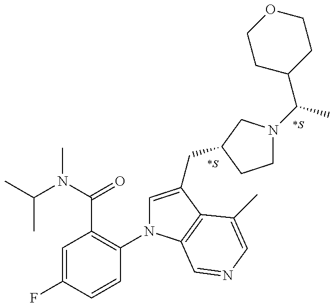 [SMILES: Cc1cncc2c1c(C[C@H]1CCN([C@@H](C)C3CCOCC3)C1)cn2-c1ccc(F)cc1C(=O)N(C)C(C)C] | |
| Compound 81 | 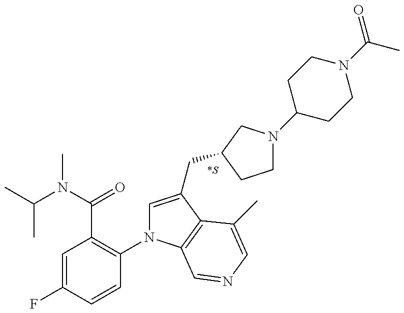 [SMILES: CC(=O)N1CCC(N2CC[C@H](Cc3cn(-c4ccc(F)cc4C(=O)N(C)C(C)C)c4cncc(C)c34)C2)CC1] | Intermediate 27 & 1- acetylpiperidin-4-one |
| Compound 109 | 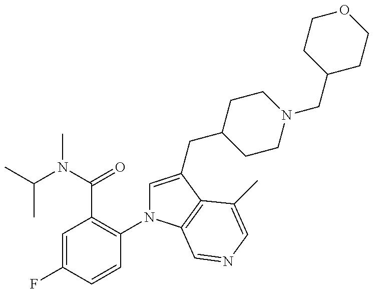 [SMILES: Cc1cncc2c1c(CC1CCN(CC3CCOCC3)CC1)cn2-c1ccc(F)cc1C(=O)N(C)C(C)C] | Intermediate 28 & tetrahydro- 2H-pyran-4-carbaldehyde |
| Compound 124 | 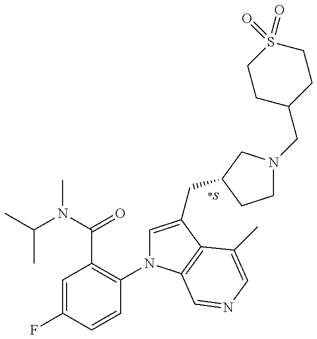 [SMILES: Cc1cncc2c1c(C[C@H]1CCN(CC3CCS(=O)(=O)CC3)C1)cn2-c1ccc(F)cc1C(=O)N(C)C(C)C] | Intermediate 27 & tetrahydro- 2H-thiopyran-4-carbaldehyde 1,1-dioxide |
| Compound 128 | 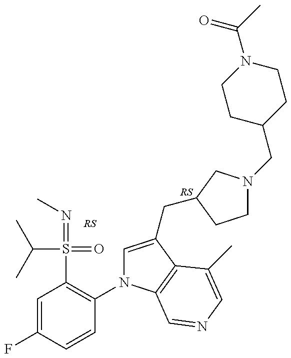 [SMILES: CN=S(=O)(c1cc(F)ccc1-n1cc(CC2CCN(CC3CCN(C(C)=O)CC3)C2)c2c(C)cncc21)C(C)C] | Intermediate 130 & 1- acetylpiperidine-4-carbaldehyde |
| Compound 128a | 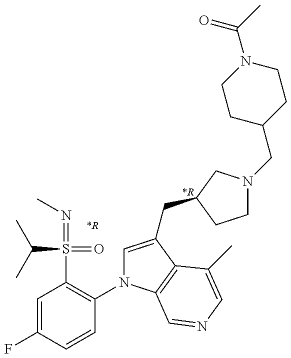 [SMILES: CN=[S@](=O)(c1cc(F)ccc1-n1cc(C[C@@H]2CCN(CC3CCN(C(C)=O)CC3)C2)c2c(C)cncc21)C(C)C] | Compound 128 was performed via Prep SFC (Stationary phase: Chiralpak Diacel AD 20 × 250 mm, Mobile phase: CO₂, iPrOH + 0.4 iPrNH₂). The first fraction was collected at Compound 128a, the second fraction as Compound 128b, the third fraction as Compound 128c and the fourth fraction as Compound 128d. |
| Compound 128b | 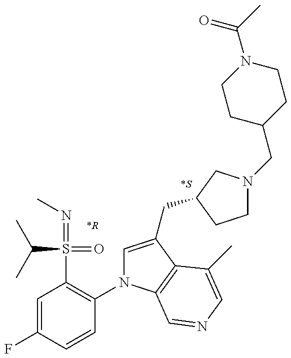 [SMILES: CN=[S@](=O)(c1cc(F)ccc1-n1cc(C[C@H]2CCN(CC3CCN(C(C)=O)CC3)C2)c2c(C)cncc21)C(C)C] | |
| Compound 128c | 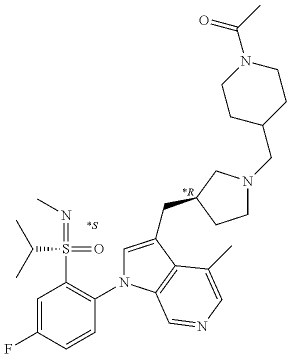 [SMILES: CN=[S@@](=O)(c1cc(F)ccc1-n1cc(C[C@@H]2CCN(CC3CCN(C(C)=O)CC3)C2)c2c(C)cncc21)C(C)C] | |
| Compound 128d | 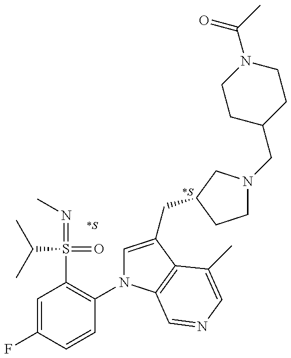 [SMILES: CN=[S@@](=O)(c1cc(F)ccc1-n1cc(C[C@H]2CCN(CC3CCN(C(C)=O)CC3)C2)c2c(C)cncc21)C(C)C] | |
| Compound 130 | 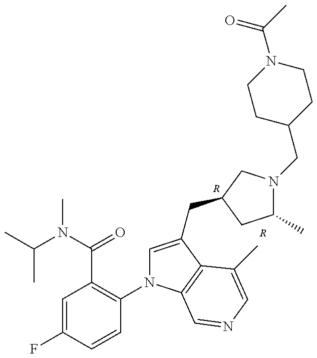 | Compound 503 & 1- acetylpiperidine-4-carbaldehyde |
| Compound 131 | 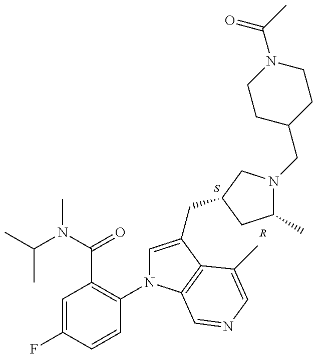 | Compound 504 & 1- acetylpiperidine-4-carbaldehyde |
| Compound 132 | 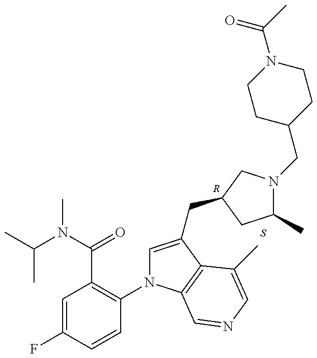 | Compound 505 & 1- acetylpiperidine-4-carbaldehyde |
| Compound 133 | 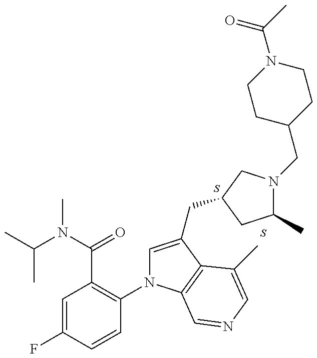 | Compound 522 & 1- acetylpiperidine-4-carbaldehyde |
| Compound 138 | 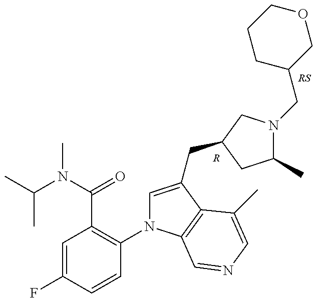 | Intermediate 202 & tetrahydro- 2H-pyran-3-carbaldehyde |
| Compound 138a & Compound 138b | 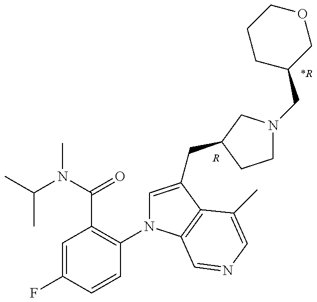 | Compound 138 was separated by Prep SFC (Stationary phase: Chiralpak Diacel AD 20 × 250 mm, Mobile phase: CO₂, EtOH- iPrOH (50-50) + 0.4% iPrNH₂). The first fraction was collected as Compound 138a & the second fraction was collected as Compound 138b. |
| Compound 139 | 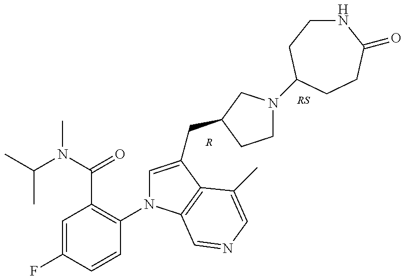 | Intermediate 202 & azepane-2,5- dione |
| Compound 139a & Compound 139b | 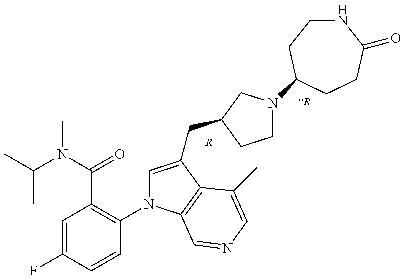 | Compound 139 was separated by Prep SFC (Stationary phase: Chiralcel Diacel IH 20 × 250 mm, Mobile phase: CO₂, MeOH + 0.4 iPrNH₂). The first fraction was collected as Compound 139a & the second fraction was Compound 139b. |
| | 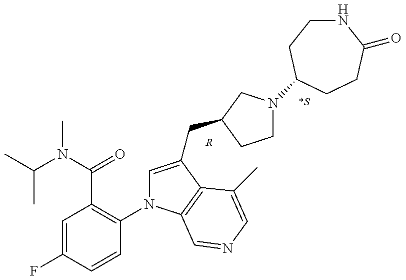 | |
| Compound 145 | 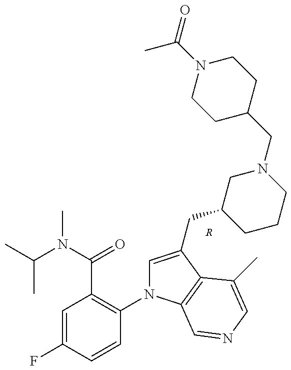 | Intermediate 213 & 1- acetylpiperidine-4-carbaldehyde |
| Compound 146 | 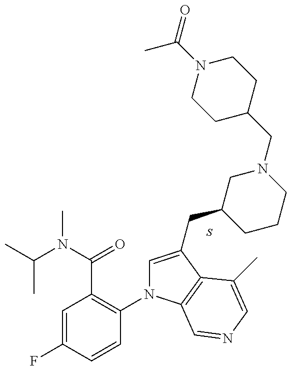 | Intermediate 215 & 1- acetylpiperidine-4-carbaldehyde |
| Compound 147a & Compound 147b | 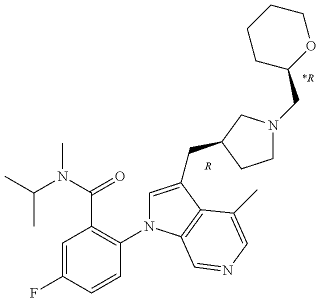 | Intermediate 202 & tetrahydro- 2H-pyran-2-carbaldehyde The product was separated by Prep SFC (Stationary phase: Chiralpak Daicel IG 20 × 250 mm, Mobile phase: CO₂, iPrOH + 0.4 iPrNH₂). The first fraction was collected as Compound 147a and the second fraction as Compound 147b. |
| | 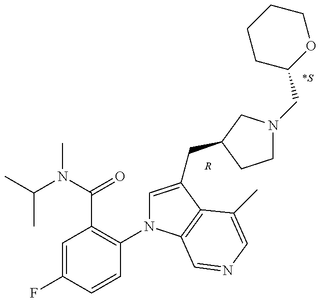 | |
| Compound 156 | 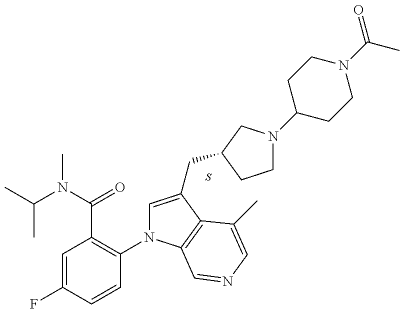 | Intermediate 225 & 1- acetylpiperidin-4-one |
| Compound 160 | 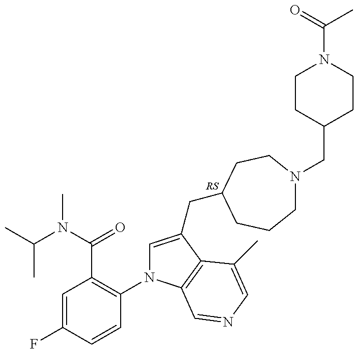 [SMILES: CC(=O)N1CCC(CN2CCCC(Cc3cn(-c4ccc(F)cc4C(=O)N(C)C(C)C)c4cncc(C)c34)CC2)CC1] | Intermediate 231 & 1- acetylpiperidine-4-carbaldehyde |
| Compound 161 | 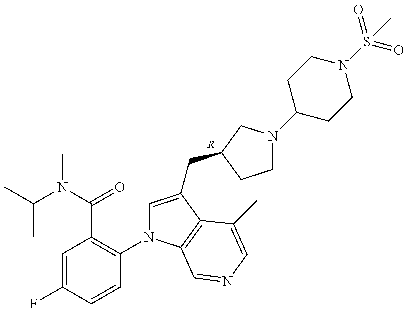 | Intermediate 20 & 1- (methylsulfonyl)piperidin-4-one |
| Compound 165a & Compound 165b | 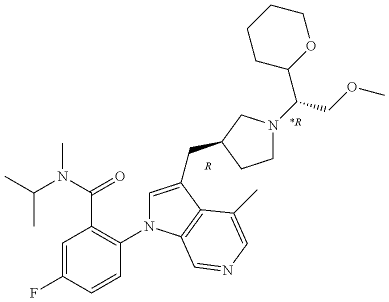 | Intermediate 202 & 2-methoxy- 1-(tetrahydro-2H-pyran-4- yl)ethan-1-one The product was separated by Prep SFC (Stationary phase: Chiralpak Daicel IG 20 × 250 mm, Mobile phase: CO₂, MeOH + 0.4 iPrNH₂). Thie first fraction was collected as Compound 165a and the second fraction as Compound 165b. |
| | 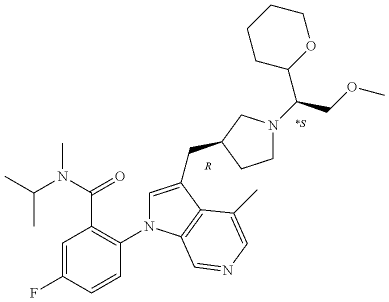 | |
| Compound 166a & Compound 166b | 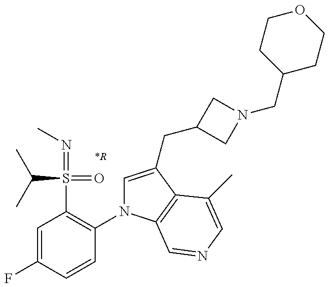 [SMILES: CN=[S@](=O)(c1cc(F)ccc1-n1cc(CC2CN(CC3CCOCC3)C2)c2c(C)cncc21)C(C)C] | Intermediate 240 & tetrahydropyran-4-carbaldehyde The product was separated via Prep SFC (Stationary phase: Chiralpak Daicel IC 20 × 250 mm, Mobile phase: CO₂, EtOH + 0.4 iPrNH₂). The first fraction was collected as Compound 166a and the second fraction as Compound 166b. |
| | 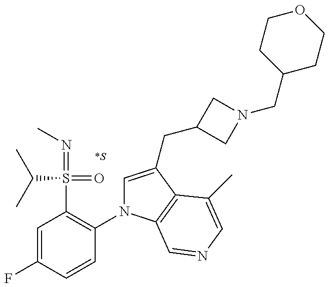 [SMILES: CN=[S@@](=O)(c1cc(F)ccc1-n1cc(CC2CN(CC3CCOCC3)C2)c2c(C)cncc21)C(C)C] | |
| Compound 167a & Compound 167b | 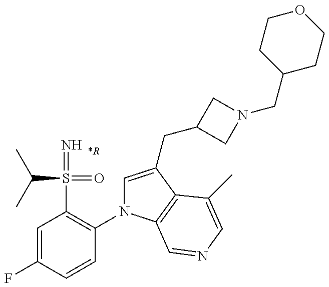 [SMILES: Cc1cncc2c1c(CC1CN(CC3CCOCC3)C1)cn2-c1ccc(F)cc1[S@](=N)(=O)C(C)C] | Intermediate 242 & tetrahydropyran-4-carbaldehyde The product was separated via Prep SFC (Stationary phase: Chiralpak Diacel AD 20 × 250 mm, Mobile phase: CO₂, iPrOH + 0.4 iPrNH₂). The first fraction was collected as Compound 167a and the second fraction as Compound 167b. |
| | 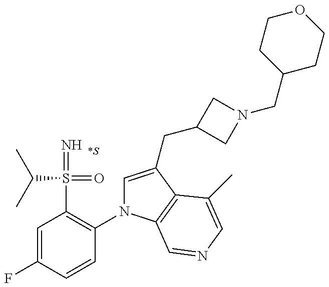 [SMILES: Cc1cncc2c1c(CC1CN(CC3CCOCC3)C1)cn2-c1ccc(F)cc1[S@@](=N)(=O)C(C)C] | |
| Compound 169 | 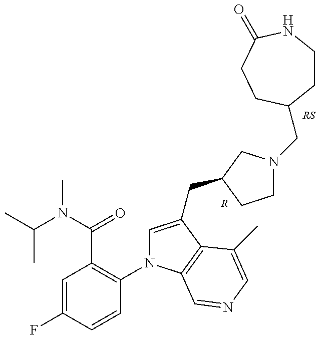 | Intermediate 202 & 7- oxoazepane-4-carbaldehyde |
| Compound 169a & Compound 169b | 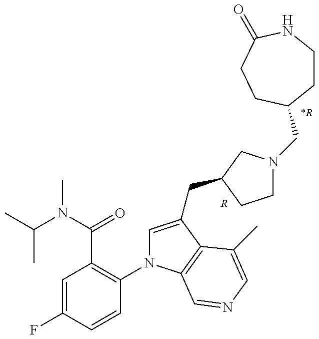 | Compound 169 was separated by Prep SFC (Stationary phase: Chiralcel Diacel IH 20 × 250 mm, Mobile phase: CO₂, EtOH + 0.4 iPrNH₂). The first fraction was collected as Compound 169a & the second fraction as Compound 169b. |
| | 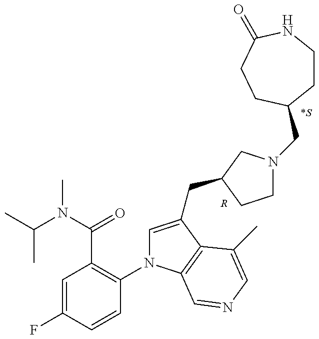 | |
| Compound 171 | 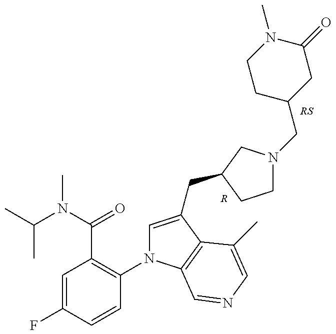 | Intermediate 202 & 1-methyl-2- oxopiperidine-4-carbaldehyde |
| Compound 180a & Compound 180b | 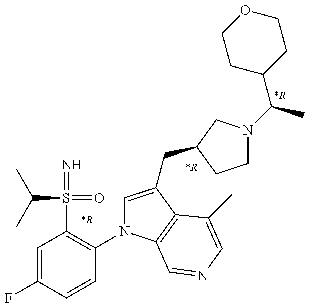 [SMILES: Cc1cncc2c1c(C[C@@H]1CCN([C@H](C)C3CCOCC3)C1)cn2-c1ccc(F)cc1[S@](=N)(=O)C(C)C] | Intermediate 131a & 1- (tetrahydro-2H-pyran-4- yl)ethenone The product was separated by Prep SFC (Stationary phase: Chiralpak Diacel AD 20 × 250 mm, Mobile phase: CO₂, EtOH- iPrOH (50-50) + 0.4% iPrNH₂). The first fraction was collected as Compound 180a and the second fraction as Compound 180b. |
| | 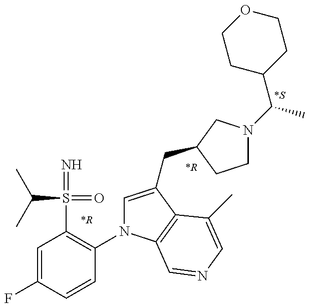 [SMILES: Cc1cncc2c1c(C[C@@H]1CCN([C@@H](C)C3CCOCC3)C1)cn2-c1ccc(F)cc1[S@](=N)(=O)C(C)C] | |
| Compound 181a & Compound 181b | 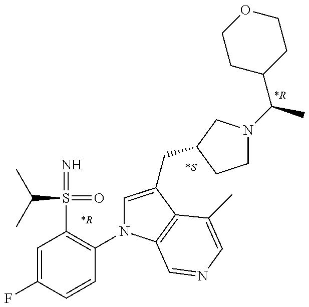 [SMILES: Cc1cncc2c1c(C[C@H]1CCN([C@H](C)C3CCOCC3)C1)cn2-c1ccc(F)cc1[S@](=N)(=O)C(C)C] | Intermediate 131b & 1- (tetrahydro-2H-pyran-4- yl)ethenone The product was separated by Prep SFC (Stationary phase: Chiralpak Diacel AD 20 × 250 mm, Mobile phase: CO₂, EtOH- iPrOH (50-50) + 0.4% iPrNH₂). The first fraction was collected as Compound 181a and the second fraction as Compound 181b. |
| | 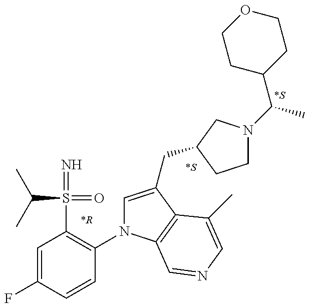 [SMILES: Cc1cncc2c1c(C[C@H]1CCN([C@@H](C)C3CCOCC3)C1)cn2-c1ccc(F)cc1[S@](=N)(=O)C(C)C] | |
| Compound 182a & Compound 182b | 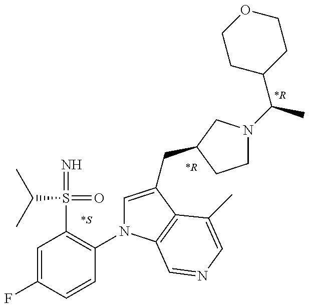 [SMILES: Cc1cncc2c1c(C[C@@H]1CCN([C@H](C)C3CCOCC3)C1)cn2-c1ccc(F)cc1[S@@](=N)(=O)C(C)C] | Intermediate 131c & 1- (tetrahydro-2H-pyran-4- yl)ethenone The product was separated by Prep SFC (Stationary phase: Chiralpak Diacel AD 20 × 250 mm, Mobile phase: CO₂, EtOH- iPrOH (50-50) + 0.4% iPrNH₂). The first fraction was collected as Compound 182a and the second fraction as Compound 182b. |
| | 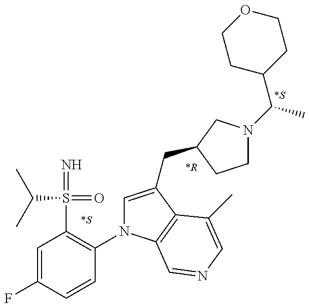 [SMILES: Cc1cncc2c1c(C[C@@H]1CCN([C@@H](C)C3CCOCC3)C1)cn2-c1ccc(F)cc1[S@@](=N)(=O)C(C)C] | |
| Compound 183a & Compound 183b | 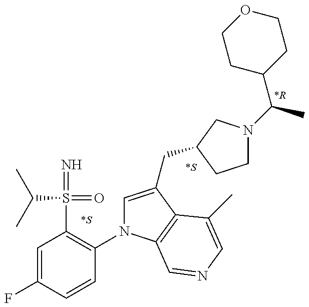 [SMILES: Cc1cncc2c1c(C[C@H]1CCN([C@H](C)C3CCOCC3)C1)cn2-c1ccc(F)cc1[S@@](=N)(=O)C(C)C] | Intermediate 131d & 1- (tetrahydro-2H-pyran-4- yl)ethenone The product was separated by Prep SFC (Stationary phase: Chiralpak Diacel AD 20 × 250 mm, Mobile phase: CO₂, EtOH- iPrOH (50-50) + 0.4% iPrNH₂) The first fraction was collected as Compound 183a and the second fraction as Compound 183b. |
| | 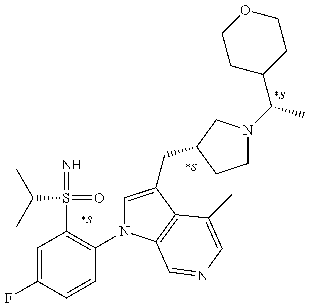 [SMILES: Cc1cncc2c1c(C[C@H]1CCN([C@@H](C)C3CCOCC3)C1)cn2-c1ccc(F)cc1[S@@](=N)(=O)C(C)C] | |
| Compound 188a & Compound 188b | 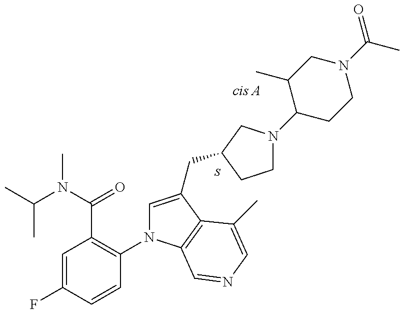 | Intermediate 225 & 1-acetyl-3- methylpiperidin-4-one The product was separated by Prep. HPLC (Column: SunFire C18 150*19 mm*5 um, Mobile PhaseA: water (0.1% NH₄OAc), Mobile Phase B: acetonitrile, Flow rate: 15 mL/min, gradient condition from 10% B to 50%). The first fraction was collected as Compound 188a and the second fraction as Compound 188b. |
| | 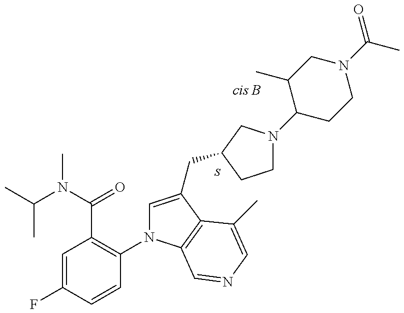 | |
| Compound 190a & Compound 190b | 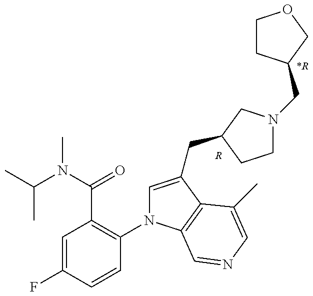 | Intermediate 202 & tetrahydrofuran-3-carbaldehyde The product was separated by Prep SFC (Stationary phase: Chiralpak Diacel AD 20 × 250 mm, Mobile phase: CO₂, EtOH- iPrOH (50-50) + 0.4% iPrNH₂). The first fraction was collected as Compound 190a and the second fraction as Compound 190b. |
| | 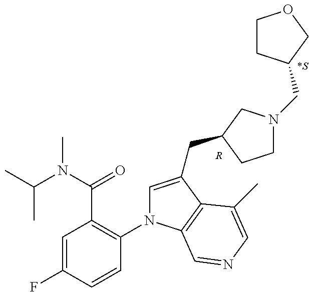 | |
| Compound 191a & Compound 191b | 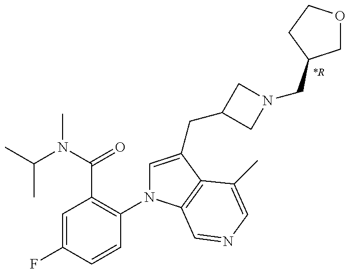 [SMILES: Cc1cncc2c1c(CC1CN(C[C@H]3CCOC3)C1)cn2-c1ccc(F)cc1C(=O)N(C)C(C)C] | Intermediate 25 & tetrahydrofuran-3-carbaldehyde The product was separated by P Prep SFC (Stationary phase: Chiralcel Diacel IH 20 × 250 mm, Mobile phase: CO₂, iPrOH + 0.4 iPrNH₂). The first fraction was collected as Compound 191a and the second fraction as Compound 191b. |
| | 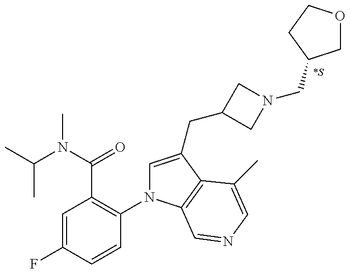 [SMILES: Cc1cncc2c1c(CC1CN(C[C@@H]3CCOC3)C1)cn2-c1ccc(F)cc1C(=O)N(C)C(C)C] | |
| Compound 193 | 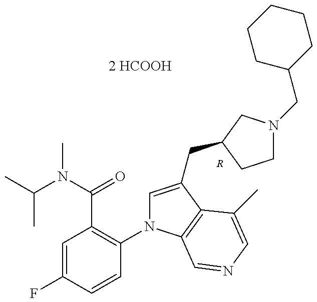 | Intermediate 20 & cyclohexanecarbaldehyde |
| Compound 194 | 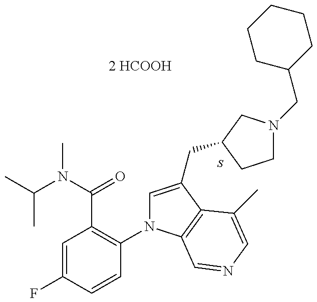 | Intermediate 225 & cyclohexanecarbaldehyde |
| Compound 196 | 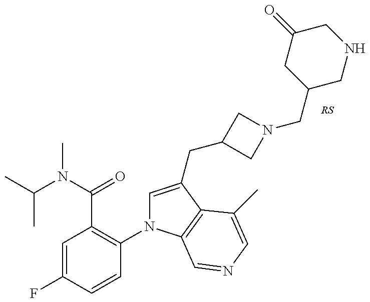 [SMILES: Cc1cncc2c1c(CC1CN(CC3CNCC(=O)C3)C1)cn2-c1ccc(F)cc1C(=O)N(C)C(C)C] | Intermediate 25 & 2- oxopiperidine-4-carbaldehyde |
| Compound 198a & Compound 198b | 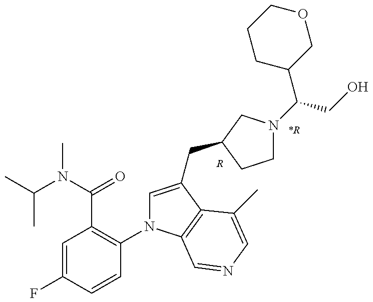 | Intermediate 202 & 259 The product was separated by Prep SFC (Stationary phase: Chiralpak Daicel IG 20 × 250 mm, Mobile phase: CO₂, EtOH + 0.4 iPrNH₂). The first fraction was collected as Compound 198a and the second as Compound 198b. |
| | 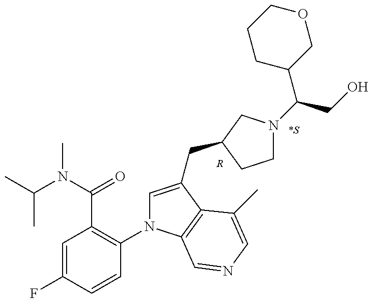 | |
| Compound 206a & Compound 206b | 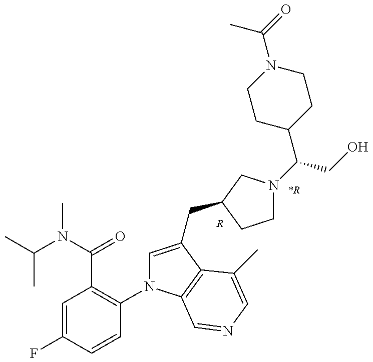 | Intermediate 202 & 266 The product was separated by Prep SFC (Stationary phase: Chiralpak Diacel AD 20 × 250 mm, Mobile phase: CO₂, EtOH + 0.4 iPrNH₂). The first fraction was collected as Compound 206a and the second as Compound 206b. |
| | 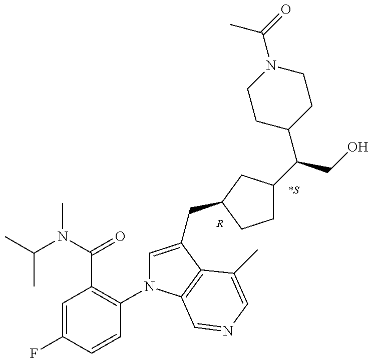 | |
| Compound 213 & Compound 213b | 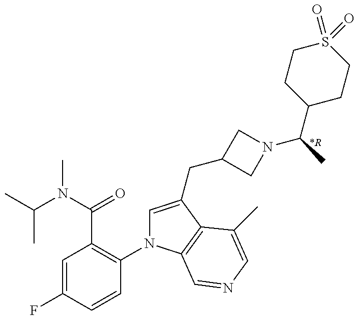 [SMILES: Cc1cncc2c1c(CC1CN([C@H](C)C3CCS(=O)(=O)CC3)C1)cn2-c1ccc(F)cc1C(=O)N(C)C(C)C] | Intermediate 25 & 1-(1,1- dioxidotetrahydro-2H-thiopyran- 4-yl)ethan-1-one The product was separated by SFC (separation condition: DAICEL CHIRALPAK IG (250 mm*30 mm, 10 um); Mobile phase: A: Supercritical CO₂, B: 0.1% NH₃H₂O MeOH, A:B = 40:60 at 80 mL/min; Column Temp: 38° C.; Nozzle Pressure: 100 Bar, Nozzle Temp: 60° C.; Evaporator Temp: 20° C.; Trimmer Temp: 25° C.; Wavelength: 220 nm). The first fraction was collected as Compound 213a and the second fraction as Compound 213b. |
| | 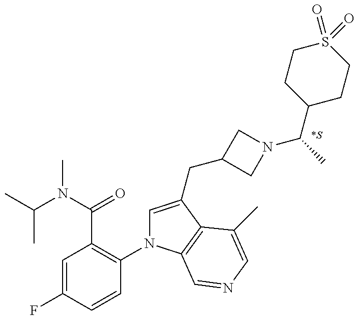 [SMILES: Cc1cncc2c1c(CC1CN([C@@H](C)C3CCS(=O)(=O)CC3)C1)cn2-c1ccc(F)cc1C(=O)N(C)C(C)C] | |
| Compound 214 | 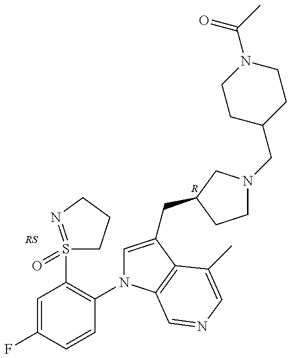 | Intermediate 278 & 1- acetylpiperidine-4-carbaldehyde Compound 214 was further separated by Prep SFC (Stationary phase: Chiralpak Diacel AD 20 × 250 mm, Mobile phase: CO₂, EtOH + 0.4 iPrNH₂). The first fraction was collected as Compound 214a and the second fraction as Compound 214b. |
| Compound 214a & Compound 214b | 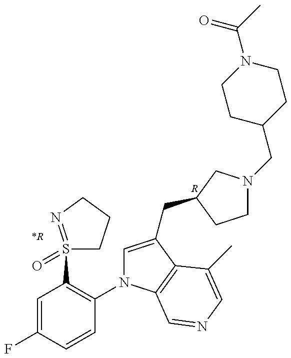 | |
| | 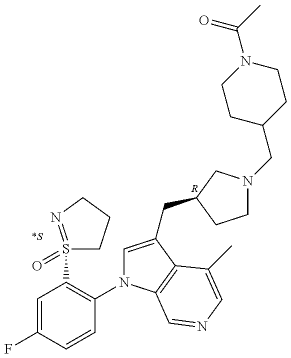 | |
| Compound 224 | 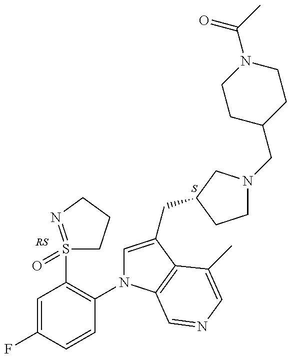 | Intermediate 298 & 1- acetylpiperidine-4-carbaldehyde Compound 224 was further separated by Prep SFC (Stationary phase: Chiralpak Diacel AD 20 × 250 mm, Mobile Phase: CO₂, EtOH + 0.4 iPrNH₂). The first fraction was collected as Compound 224a and the second fraction as Compound 224b. |
| Compound 224a & Compound 224b | 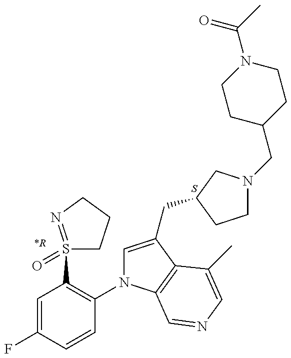 | |
| | 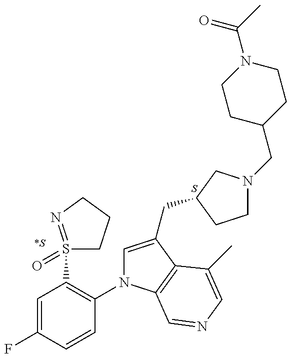 | |
| Compound 225 | 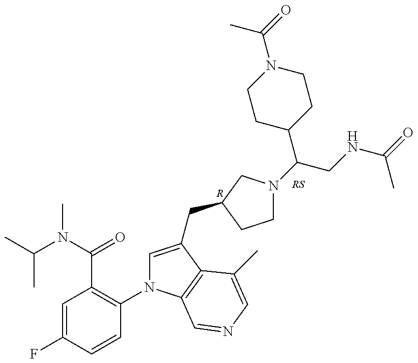 | Intermediate 202 & 300 |
| Compound 226 | 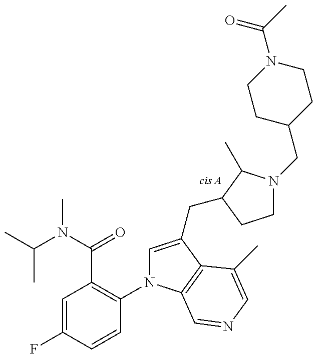 | Compound 507 & 1- acetylpiperidine-4-carbaldehyde |
| Compound 227 | 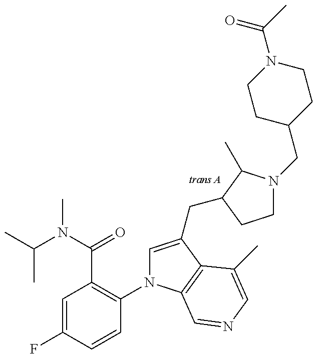 | Compound 508 & 1- acetylpiperidine-4-carbaldehyde |
| Compound 228 | 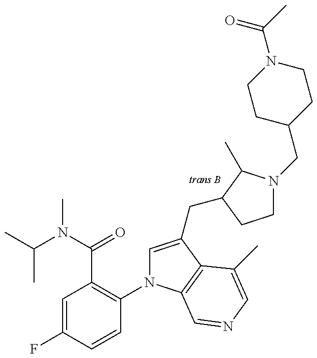 | Compound 509 & 1- acetylpiperidine-4-carbaldehyde |
| Compound 229 | 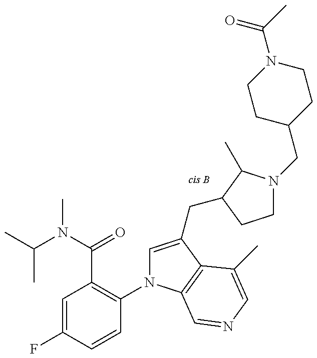 | Compound 510 & 1- acetylpiperidine-4-carbaldehyde |
| Compound 230a & Compound 230b | 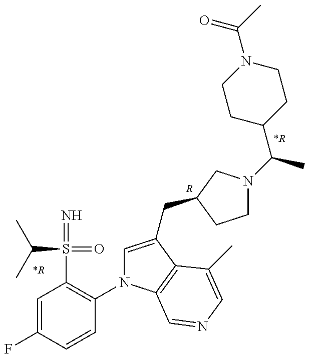 | Intermediate 311a & 1-(4- acetylpiperidino)ethan-1-one The product was separated by Prep SFC (Stationary phase: Chiralpak Diacel AD 20 × 250 mm, Mobile phase: CO₂, EtOH + 0.4 iPrNH₂). The first fraction was collected as Compound 230a & the second fraction as Compound 230b. |
| | 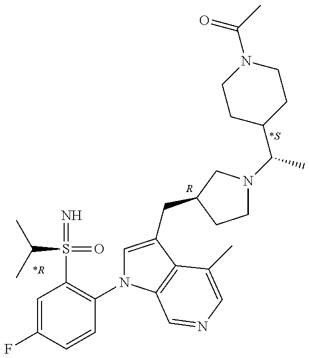 | |
| Compound 231a & Compound 231b | 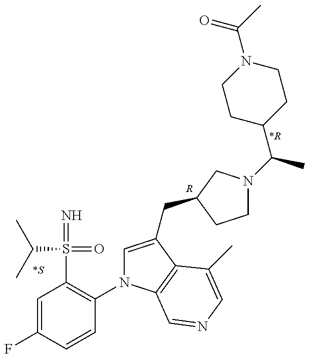 | Intermediate 311b & 1-(4- acetylpiperidino)ethan-1-one The product was separated by Prep SFC (Stationary phase: Chiralpak Diacel AD 20 × 250 mm, Mobile phase: CO₂, EtOH- iPrOH (50-50) + 0.4% iPrNH₂). The first fraction was collected as Compound 231a & the second fraction as Compound 231b. |
| | 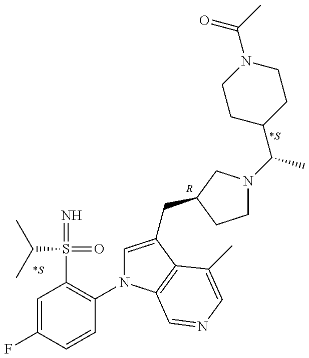 | |
| Compound 232a & Compound 232b | 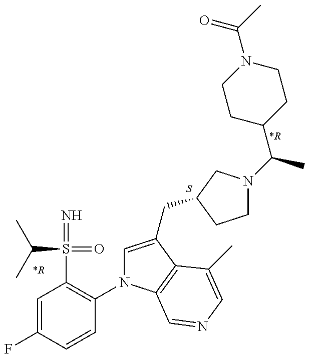 | Intermediate 312a & 1-(4- acetylpiperidino)ethan-1-one The product was separated by Prep SFC (Stationary phase: Chiralpak Diacel AD 20 × 250 mm, Mobile phase: CO₂, EtOH + 0.4 iPrNH₂). The first fraction was collected as Compound 232a & the second fraction as Compound 232b. |
| | 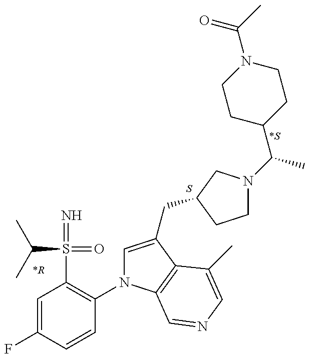 | |
| Compound 233a & Compound 233b | 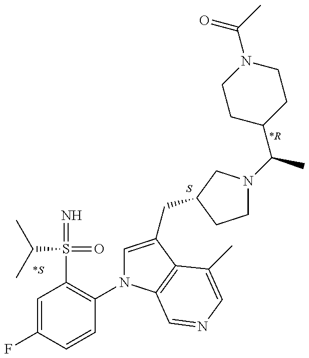 | Intermediate 312b & 1-(4- acetylpiperidino)ethan-1-one The product was separated by Prep SFC (Stationary phase: Chiralpak Diacel AD 20 × 250 mm, Mobile phase: CO₂, EtOH- iPrOH (50-50) + 0.4% iPrNH₂). The first fraction was collected as Compound 233a & the second fraction as Compound 233b. |
| | 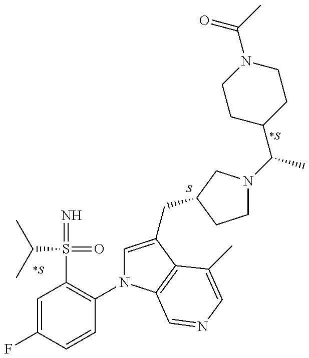 | |
| Compoudn 239 | 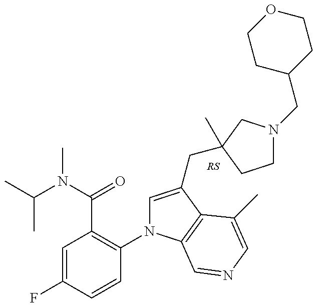 | Compound 511 & tetrahydro- 2H-pyran-4-carbaldehyde |
| Compound 240a & Compound 240b | 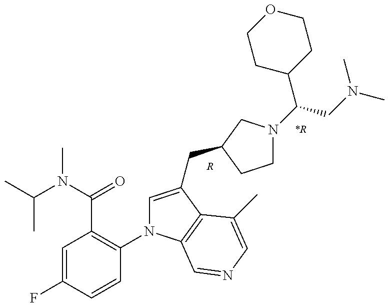 | Intermediate 20 & 324 The product was separated by Prep SFC (Stationary phase: Chiralpak Daicel IG 20 × 250 mm, Mobile phase: CO₂, EtOH + 0.4 iPrNH₂). The first fraction was collected as Compound 240a & the second fraction as Compound 240b. |
| | 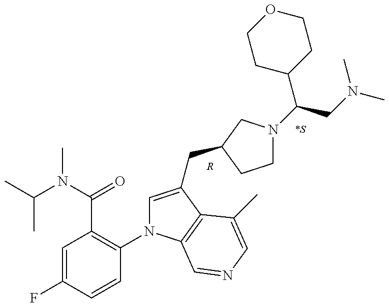 | |
| Compound 252a & Compound 252b | 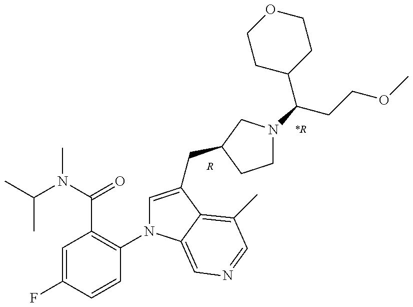 | Intermediate 20 & 334 The product was separated by Chiral Prep. HPLC (separation condition: Column: Chiralpak IA 5 um 30 * 250 mm, Mobile Phase: Hexane: iso-Propyl alcohol = 90:10 at 325 mL/min; Temp: 30° C.; Wavelength: 254 nm). The first fraction was collected as Compound 252a & the second fraction as Compound 252b. |
| | 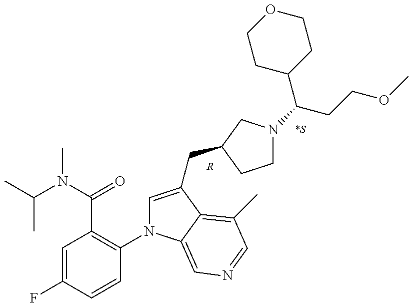 | |
| Compound 253a & Compound 253b | 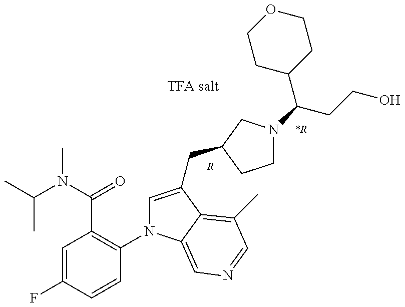 | Intermediate 202 & 335 The product was separated by chiral Prep. HPLC (separation condition: Column: Chiralpak IA 5 um 30 * 250 mm; Mobile Phase: Hexane: EtOH = 80:20 at 25 mL/min; Temp: 30° C.; Wavelength: 254 nm). The first fraction was collected as Compound 253a & the second fraction as Compound 253b. |
| | 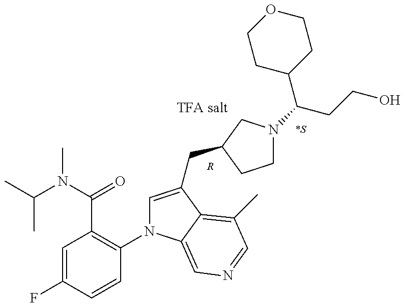 | |
| Compound 257 | 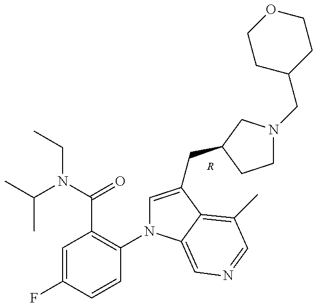 | Intermediate 338 & tetrahydro- 2H-pyran-4-carbaldehyde |
| Compound 523a (E or Z, not determined) & Compound 523b (Z or E, not determined) | 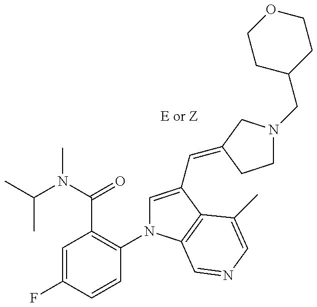 [SMILES: Cc1cncc2c1c(/C=C1\CCN(CC3CCOCC3)C1)cn2-c1ccc(F)cc1C(=O)N(C)C(C)C] | Intermediate 399 & tetrahydro- 2H-pyran-4-carbaldehyde |
| | 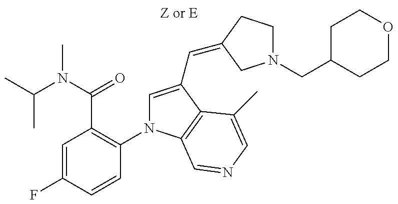 [SMILES: Cc1cncc2c1c(/C=C1/CCN(CC3CCOCC3)C1)cn2-c1ccc(F)cc1C(=O)N(C)C(C)C] | |
| Compound 524a (E or Z, not determined) & Compound 524b (Z or E, not determined) | 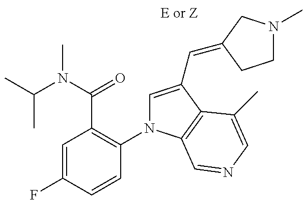 [SMILES: Cc1cncc2c1c(/C=C1\CCN(C)C1)cn2-c1ccc(F)cc1C(=O)N(C)C(C)C] | Intermediate 399 & 37% aq. Formaldehyde solution |
| | 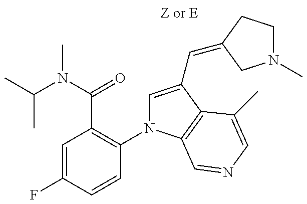 [SMILES: Cc1cncc2c1c(/C=C1/CCN(C)C1)cn2-c1ccc(F)cc1C(=O)N(C)C(C)C] | |

### Preparation of Compound 50:

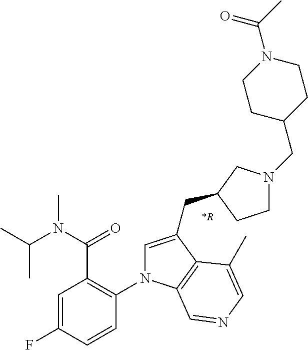 [SMILES: CC(=O)N1CCC(CN2CC[C@@H](Cc3cn(-c4ccc(F)cc4C(=O)N(C)C(C)C)c4cncc(C)c34)C2)CC1]

To a solution of Compound 381 (70 mg, 0.102 mmol) and DIEA (79 mg, 0.61 mmol) in DCM (4 mL) was added acetic anhydride (52 mg, 0.51 mmol). After stirring at r.t. for 4 hours, the reaction mixture was concentrated, and the residue was purified by Prep-HPLC: Waters Xbridge C18 5 μm 19*150 mm. Mobile phase A:0.1% NH₄OH+10 mM NH₄HCO₃ in water. B: CH₃CN, gradient from 0% B to 100% B. The pure fraction was collected and lyophilized to afford Compound 50 (50 mg, 88% yield) as a white solid.

### Preparation of Compound 51:

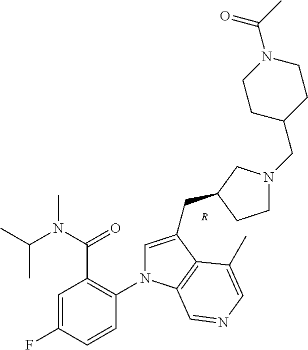

At 0° C., to a solution of Compound 485 (1.04 g, 95% purity, 1.95 mmol) in DCM (10 mL) was added acetyl chloride (160 mg, 2.05 mmol) and triethylamine (592 mg, 5.85 mmol). After stirring at room temperature for 2 hours, the resulting mixture was poured into water and extracted with dichloromethane (20 mL) twice. The combined organic layers were washed with brine (20 mL), dried over Na₂SO₄, filtered and concentrated to afford the crude product, which was purified by prep HPLC (Column: Xbridge C18 150*19 mm*5 um, Mobile Phase A: water (0.1% NH₄HCO₃), Mobile Phase B: acetonitrile, Flow rate: 15 mL/min, gradient condition from 15% B to 60% B). The collected fraction was lyophilized to give Compound 51 (1.25 g, 99.8% purity, 74.9% yield) as a white solid.

Alternatively, compound 51 can also be prepared with the following procedure:

Intermediate 202 (as 0.2TFA salt) (0.20 g, 0.49 mmol) and 1-acetylpiperidine-4-carbaldehyde (0.097 g, 0.62 mmol) were dissolved in MeOH (5.5 mL). After stirring at ambient temperature for −5 min, solid NaCNBH₃ (0.039 g, 0.62 mmol) was added. The resulting mixture was stirred at ambient temperature for ˜2 h, after which sat. aq. NaHCO₃ solution was added. Then, most of the MeOH was evaporated to dryness, and DCM was added. The pH of the water layer was adjusted to pH>10 with 1M aq. NaOH solution. The layers were separated and the water layer was extracted three times more with DCM. The organic layers were combined, dried over Na₂SO₄, filtered and evaporated. The residue was purified by silica gel column chromatography eluting with methanol (+1% 7N NH₃ in MeOH) in dichloromethane from 0% to 10% to give compound 51 (0.060 g, 0.11 mmol, 35% yield).

Compound 51 (originating from route via intermediate 202; 0.051 g, purity 99.7%, LC/MS method 32) was dissolved in 2-3 drops of isopropylacetate (IPAC), after which the resulting solution was stirred at 45° C. for ˜5 h. Next, the mixture was allowed to stir at ambient temperature for 48 h, after which it was filtered to obtain a white solid material corresponding with Compound 51 in its crystalline free base Form. Melting point (via DSC): Tₒₙₛₑₜ=121.6° C.

Compound 51 ((originating from route via intermediate 202; ˜1 g, 98.7% purity, LC/MS method 33) was dissolved in cyclopentylmethylether (CPME) (3 mL), after which heptane (2 mL) was slowly added, followed by the addition of −10 mg of seeding crystals (obtained via previous procedure). Next, 1 mL of heptane was added and the mixture stirred for 20 h, after which the suspension was filtered to give solid material which was dried at 40° C. under vacuum to yield Compound 51 in its crystalline free base Form (96% yield).

Chiral SFC method 1 was employed to match the stereochemistry of compound 51 obtained through the route employing Compound 485 or intermediate 202; retention time=4.73-4.77 min.

### Preparation of Compound 51a:

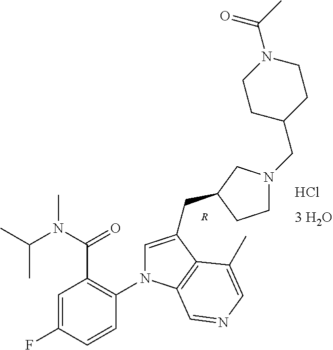

Compound 51 (0.50 g, 0.91 mmol, purity 95.2% (determined by LC/MS method 32)) was dissolved in acetone (0.50 mL) and stirred to give a clear solution. Next, a solution of 1M HCl in acetone was prepared as follows: 1 mL of concentrated aq. HCl solution was added to 11 mL of acetone. Then, a solution of 1M HCl in acetone (0.92 mL, 1 eq.) was added, keeping a solution. The solution was stirred at ambient temperature for ˜30-60 min, after which heptane (5.0 mL) was added. Next, acetone was added (3.0 mL). Vigorous stirring was initiated, and the mixture was stirred overnight. Then, a fine white suspension was obtained, and the suspension was filtered. The solid was rinsed with heptane and dried to give Compound 51a as a mono HCl trihydrate salt (when determined via dynamic vapor sorption analysis around 3 equivalents water) as a white solid (0.48 g, yield 78%). Melting point (via DSC): Tₒₙₛₑₜ=139° C.

Compound 51a was obtained as a variable hydrate with equilibrated water content varying as function of humidity —mainly trihydrate at ambient % relative humidity.

The Following Compounds were Synthesized by an Analogous Method as Described for Compound 51

Alternatively, compounds can also be purified by the following method: prep. HPLC: (Column: Waters Sunfire C18 5 μm, 19*150 mm, Mobile Phase A: water (0.1% HCOOH), Mobile Phase B: acetonitrile, Flow rate: 17 mL/min, gradient condition from 0% B to 20% B).

| Compound No. | Structure | Starting Materials |
|---|---|---|
| Compound 58 | 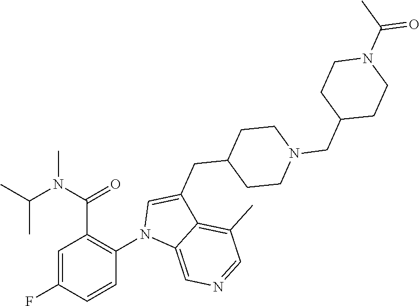 [SMILES: CC(=O)N1CCC(CN2CCC(Cc3cn(-c4ccc(F)cc4C(=O)N(C)C(C)C)c4cncc(C)c34)CC2)CC1] | Compound 433 & acetyl chloride |
| Compound 90a & Compound 90b | 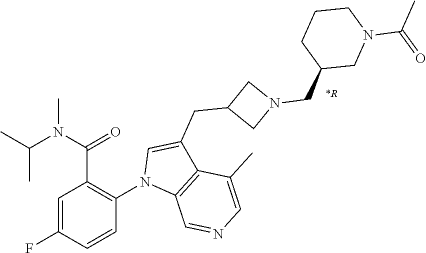 [SMILES: CC(=O)N1CCC[C@H](CN2CC(Cc3cn(-c4ccc(F)cc4C(=O)N(C)C(C)C)c4cncc(C)c34)C2)C1] | Compound 431 & acetyl chloride A purification was performed via Prep SFC (Stationary phase: Chiralpak Diacel AD 20 × 250 mm, Mobile phase: CO₂, iPrOH + 0.4 iPrNH₂) The first fraction was collected as Compound 90a and the second as Compound 90b |
| | 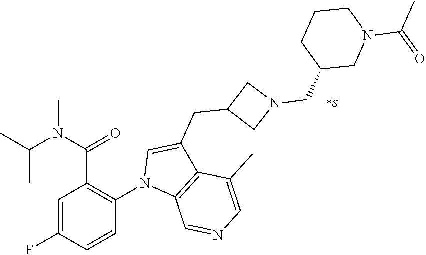 [SMILES: CC(=O)N1CCC[C@@H](CN2CC(Cc3cn(-c4ccc(F)cc4C(=O)N(C)C(C)C)c4cncc(C)c34)C2)C1] | |
| Compound 102 | 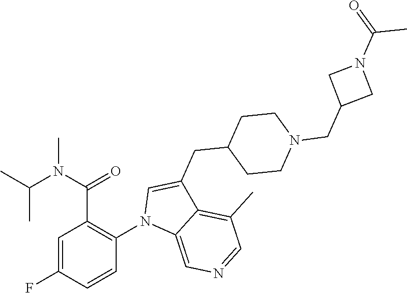 [SMILES: CC(=O)N1CC(CN2CCC(Cc3cn(-c4ccc(F)cc4C(=O)N(C)C(C)C)c4cncc(C)c34)CC2)C1] | Compound 436 & acetyl chloride |
| Compound 121 | 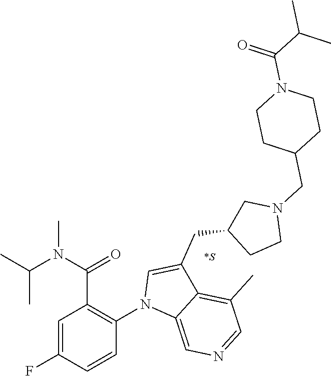 [SMILES: Cc1cncc2c1c(C[C@H]1CCN(CC3CCN(C(=O)C(C)C)CC3)C1)cn2-c1ccc(F)cc1C(=O)N(C)C(C)C] | Compound 485 & isobutyryl chloride |
| Compound 122 | 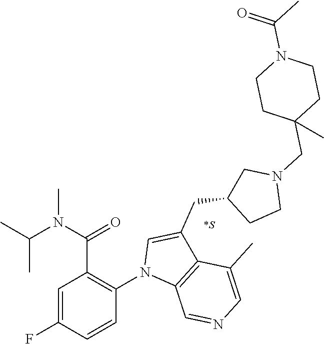 [SMILES: CC(=O)N1CCC(C)(CN2CC[C@H](Cc3cn(-c4ccc(F)cc4C(=O)N(C)C(C)C)c4cncc(C)c34)C2)CC1] | Compound 445 & acetyl chloride |
| Compound 135 | 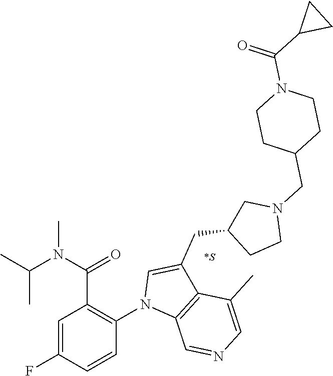 [SMILES: Cc1cncc2c1c(C[C@H]1CCN(CC3CCN(C(=O)C4CC4)CC3)C1)cn2-c1ccc(F)cc1C(=O)N(C)C(C)C] | Compound 485 & cyclopropanecarbonyl chloride |
| Compound 136 | 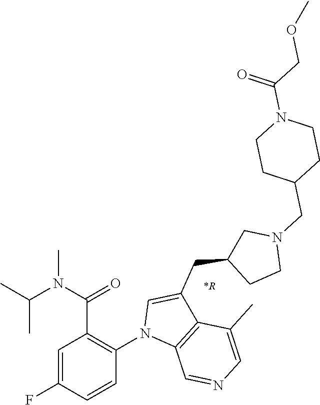 [SMILES: COCC(=O)N1CCC(CN2CC[C@@H](Cc3cn(-c4ccc(F)cc4C(=O)N(C)C(C)C)c4cncc(C)c34)C2)CC1] | Compound 381 & 2- methoxyacetyl chloride |
| Compound 149 | 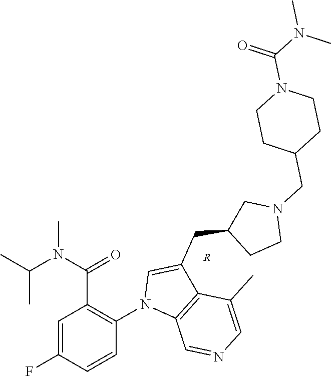 | Compound 448 & dimethylcarbamic chloride |
| Compound 170 | 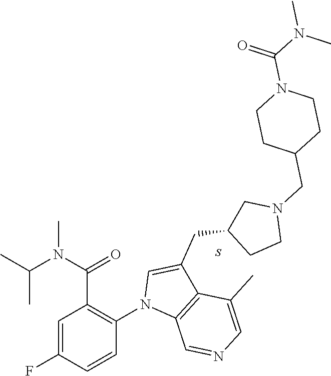 | Compound 451 & dimethylcarbamic chloride |
| Compound 173 | 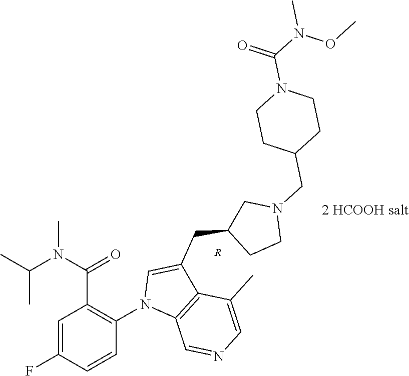 | Compound 448 & methoxy(methyl)carbamic chloride |
| Compound 174 | 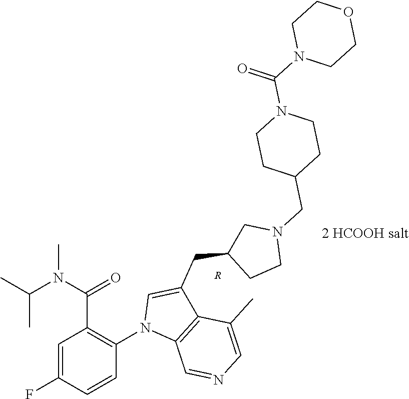 | Compound 448 & morpholine-4- carbonyl chloride |
| Compound 187 | 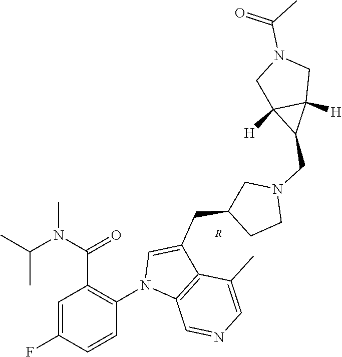 | Compound 455 & acetyl chloride |
| Compound 200 | 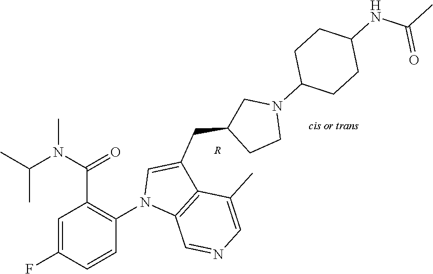 | Compound 406b & acetyl chloride |
| Compound 201 | 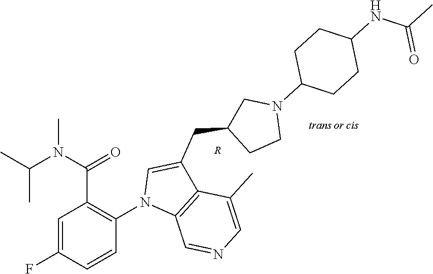 | Compound 406a & acetyl chloride |
| Compound 203 | 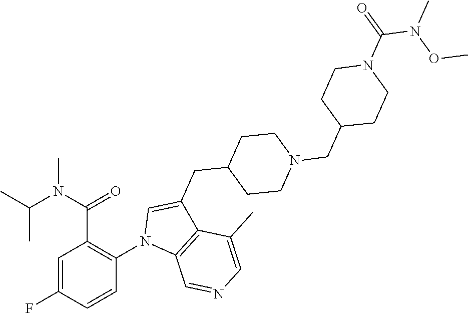 [SMILES: CON(C)C(=O)N1CCC(CN2CCC(Cc3cn(-c4ccc(F)cc4C(=O)N(C)C(C)C)c4cncc(C)c34)CC2)CC1] | Compound 433 & methoxy(methyl)carbamic chloride |
| Compound 204 | 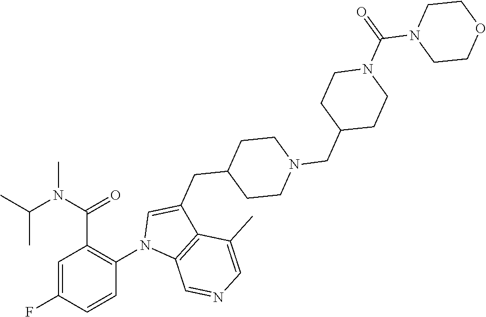 [SMILES: Cc1cncc2c1c(CC1CCN(CC3CCN(C(=O)N4CCOCC4)CC3)CC1)cn2-c1ccc(F)cc1C(=O)N(C)C(C)C] | Compound 433 & morpholine-4- carbonyl chloride |
| Compound 208 | 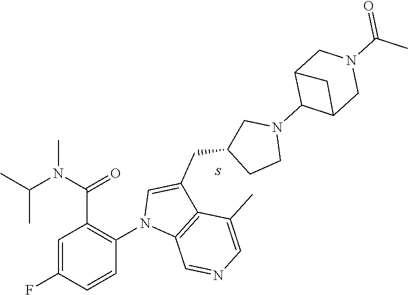 | Compound 462 & acetyl chloride |
| Compound 209 | 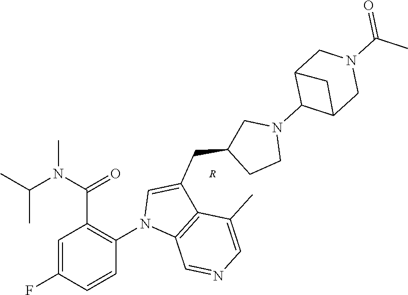 | Compound 463 & acetyl chloride |
| Compound 210 | 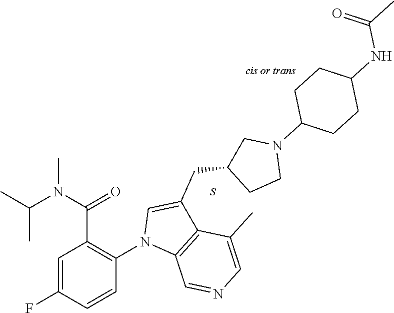 | Compound 465 & acetyl chloride |
| Compound 211 | 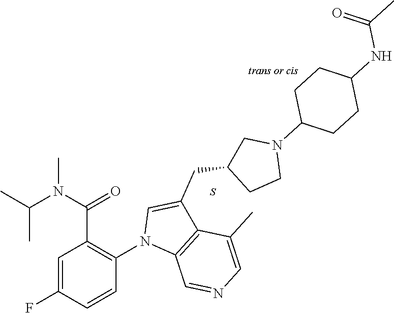 | Compound 464 & acetyl chloride |
| Compound 216 | 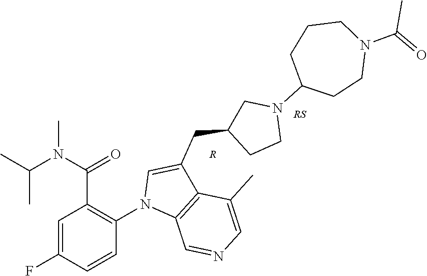 | Compound 467 & acetyl chloride |
| Compound 220 | 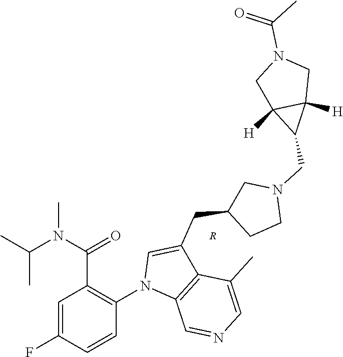 | Compound 470 & acetyl chloride |
| Compound 222 | 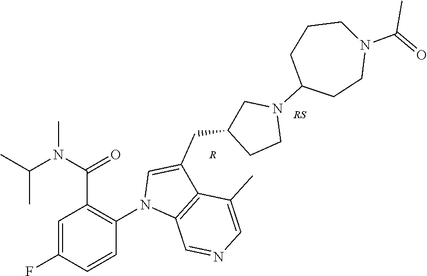 | Compound 471 & acetyl chloride |
| Compound 223 | 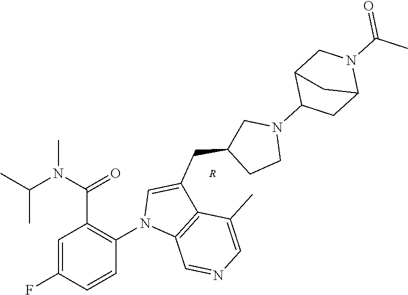 | Compound 472 & acetyl chloride |
| Compound 234 | 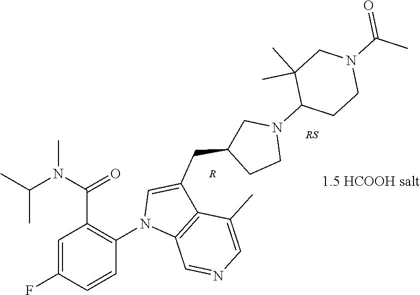 | Compound 473 & acetyl chloride |
| Compound 235 | 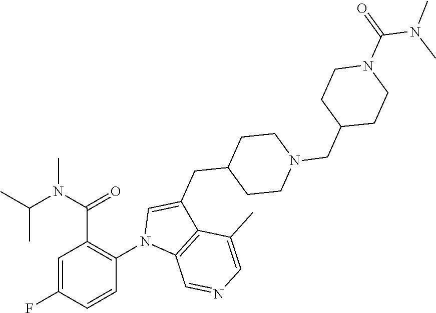 [SMILES: Cc1cncc2c1c(CC1CCN(CC3CCN(C(=O)N(C)C)CC3)CC1)cn2-c1ccc(F)cc1C(=O)N(C)C(C)C] | Compound 433 & dimethylcarbamic chloride |
| Compound 238 | 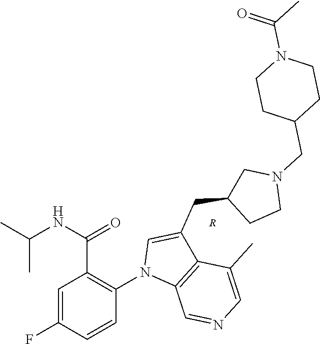 | Compound 474 & acetyl chloride |
| Compound 255 | 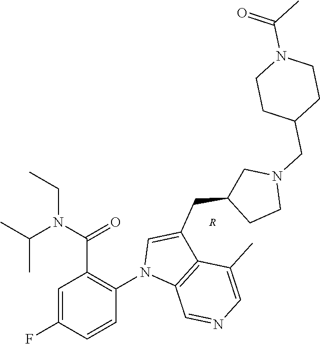 | Compound 480 & acetyl chloride |
| Compound 256 | 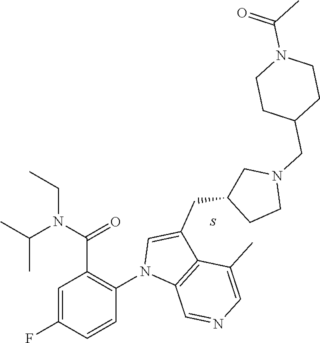 | Compound 481 & acetyl chloride |
| Compound 258 | 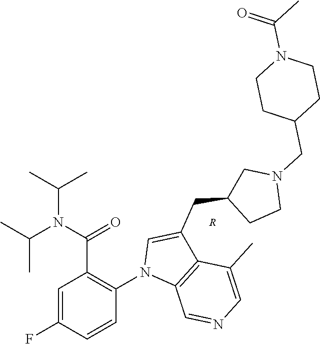 | Compound 482 & acetyl chloride |
| Compound 259 | 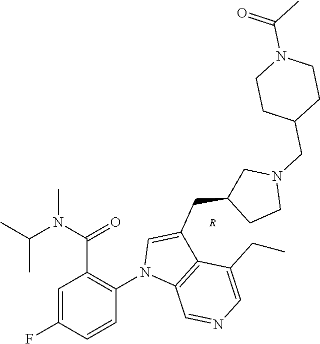 | Compound 483 & acetyl chloride |
| Compound 260 | 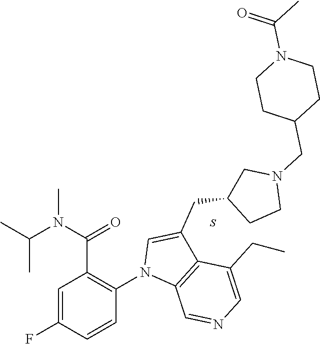 | Compound 484 & acetyl chloride |

### Preparation of Compound 59:

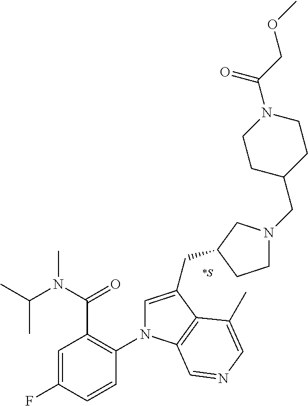

To a mixture of Compound 485 (70 mg, 0.138 mmol), methoxyacetic acid (18.7 mg, 0.208 mmol) and DIPEA (0.07 mL, 0.42 mmol) in DCM (4.2 mL) was added HATU (78.9 mg, 0.208 mmol). After stirring at rt for 16 hours, the reaction mixture was concentrated and the residue was purified by Prep. HPLC (Column: Waters Xbridge C18 5 μm, 19*150 mm, Mobile Phase A: water (0.1% NH₃H₂O+10 mM NH₄HCO₃), Mobile Phase B: acetonitrile, Flow rate: 17 mL/min, gradient condition from 30% B to 50% B). The pure fraction was collected and lyophilized to dryness to afford Compound 59 (65 mg, 79.6% yield).

### Preparation of Compound 60:

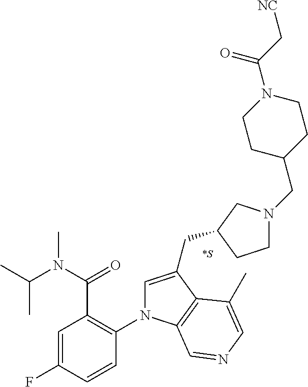

To a mixture of Compound 485, (70 mg, 0.138 mmol), cyanoacetic acid (17.7 mg, 0.208 mmol) and DIPEA (0.07 mL, 0.415 mmol) in DCM (5 mL) was added HATU (78.9 mg, 0.208 mmol). After stirring at RT for 16 hours, the reaction mixture was concentrated, and the residue was purified by Prep. HPLC (Column: Waters Xbridge C18 5 μm, 19*150 mm, Mobile Phase A: water (0.1% NH₃H₂O+10 mM NH₄HCO₃), Mobile Phase B: acetonitrile, Flow rate: 17 mL/min, gradient condition from 30% B to 50% B). The pure fraction was collected and lyophilized to dryness to afford Compound 60 (65 mg, 81% yield).

The Following Compounds were Synthesized by an Analogous Method as Described for Compound 60

Alternatively, purification can also be performed using the following method: Prep. HPLC (Column: Xbrige C18 150*19 mm*5 um, mobile phase A: water (0.1% HCOOH), mobile phase B: acetonitrile, flow rate: 15 mL/min, gradient condition from 5% B to 60% B)

| Compound No. | Structure | Starting Materials |
|---|---|---|
| Compound 82 | 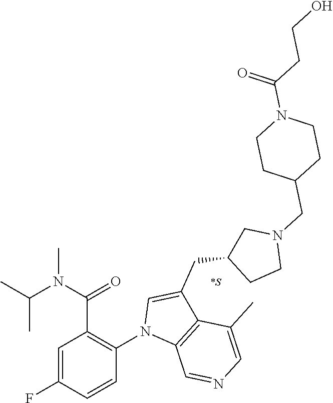 | Compound 485 & 3- hydroxypropanoic acid |
| Compound 85 | 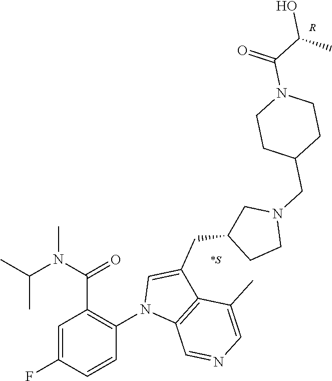 | Compound 485 & (R)-2- hydroxypropanoic acid |
| Compound 86 | 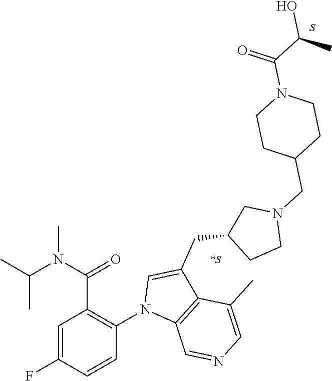 | Compound 485 & (S)-2- hydroxypropanoic acid |
| Compound 87 | 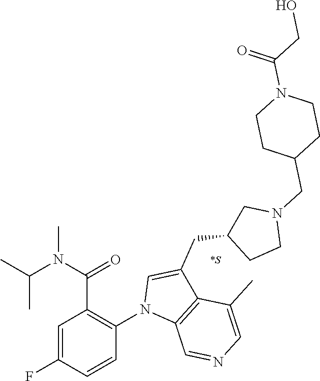 | Compound 485 & 2- hydroxyacetic acid |
| Compound 106 | 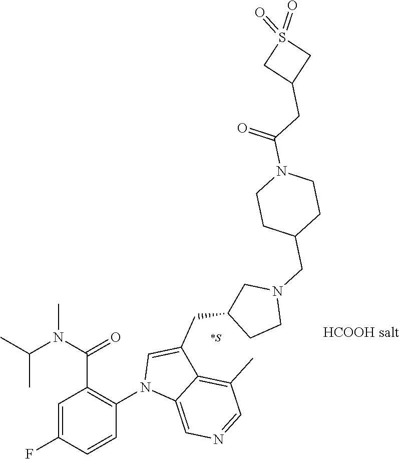 | Compound 485 & 2-(1,1- dioxidothietan-3-yl)acetic acid |
| Compound 108 | 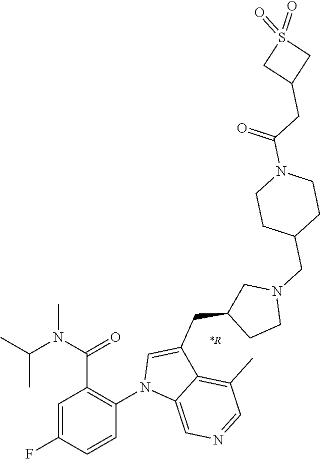 | Compound 381 & 2-(1,1- dioxidothietan-3-yl)acetic acid |
| Compound 111 | 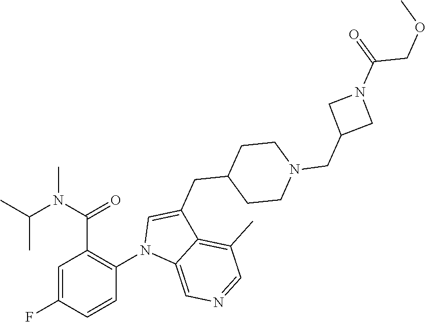 [SMILES: COCC(=O)N1CC(CN2CCC(Cc3cn(-c4ccc(F)cc4C(=O)N(C)C(C)C)c4cncc(C)c34)CC2)C1] | Compound 436 & 2- methoxyacetic acid |
| Compound 117a | 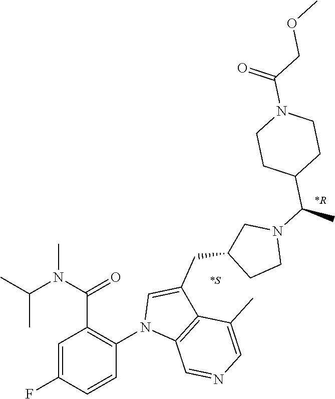 | Compound 442 & 2- methoxyacetic acid |
| Compound 117b | 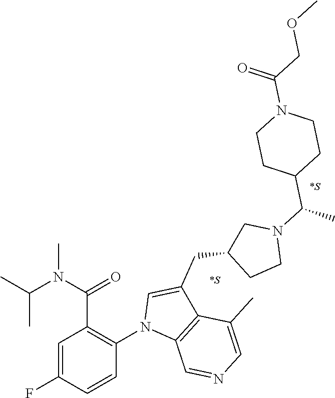 | Compound 443 & 2- methoxyacetic acid |
| Compound 123 | 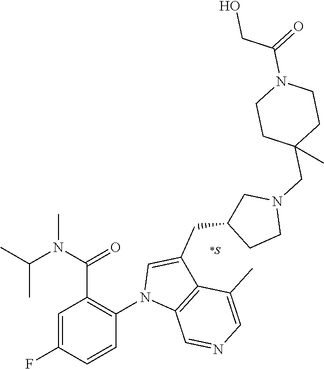 | Compound 445 & 2- hydroxyacetic acid |
| Compound 125 | 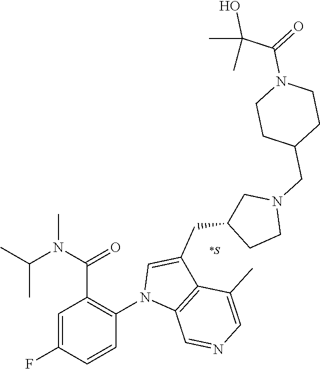 | Compound 485 & 2-hydroxy-2- methylpropanoic acid |
| Compound 126 | 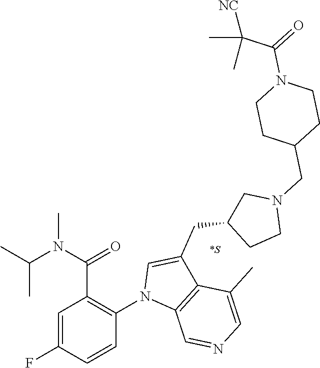 | Compound 485 & 2-cyano-2- methylpropanoic acid |
| Compound 177 | 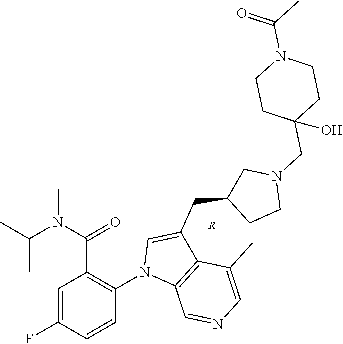 | Compound 454 & acetic acid |
| Compound 237 | 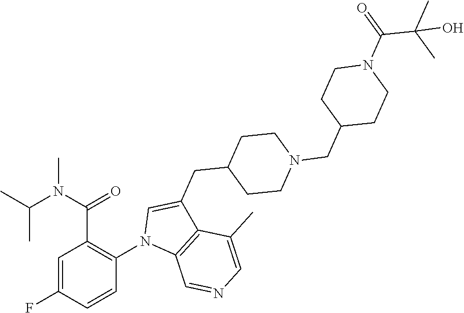 [SMILES: Cc1cncc2c1c(CC1CCN(CC3CCN(C(=O)C(C)(C)O)CC3)CC1)cn2-c1ccc(F)cc1C(=O)N(C)C(C)C] | Compound 433 & 2-hydroxy-2- methylpropanoic acid |
| Compound 244 | 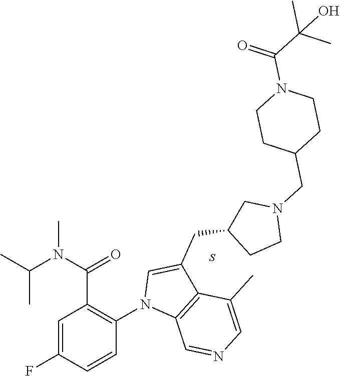 | Compound 451 & 2-hydroxy-2- methylpropanoic acid |
| Compound 245 | 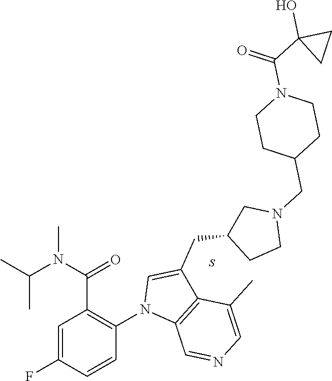 | Compound 451 & 1- hydroxycyclopropane-1- carboxylic acid |
| Compound 248 | 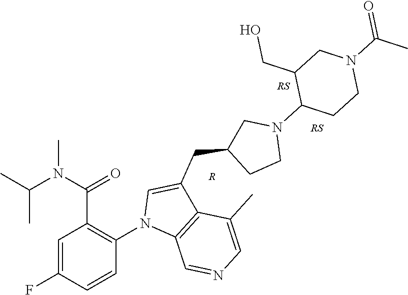 | Compound 479 & acetic acid |
| Compound 249 | 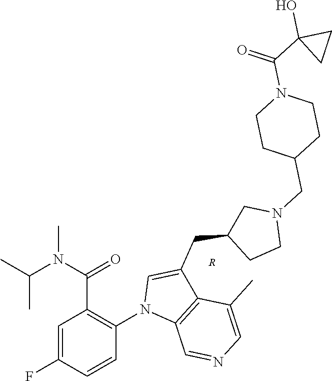 | Compound 448 & 1- hydroxycyclopropane-1- carboxylic acid |
| Compound 250 | 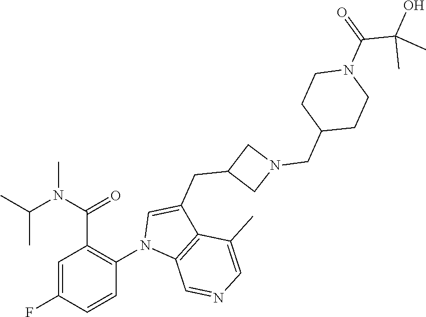 [SMILES: Cc1cncc2c1c(CC1CN(CC3CCN(C(=O)C(C)(C)O)CC3)C1)cn2-c1ccc(F)cc1C(=O)N(C)C(C)C] | Compound 429 & 2-hydroxy-2- methylpropanoic acid |
| Compound 251 | 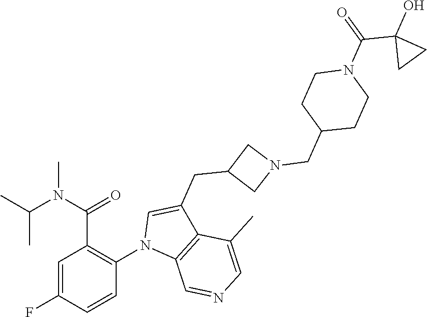 [SMILES: Cc1cncc2c1c(CC1CN(CC3CCN(C(=O)C4(O)CC4)CC3)C1)cn2-c1ccc(F)cc1C(=O)N(C)C(C)C] | Compound 429 & 1- hydroxycyclopropane-1- carboxylic acid |

### Preparation of Compound 61:

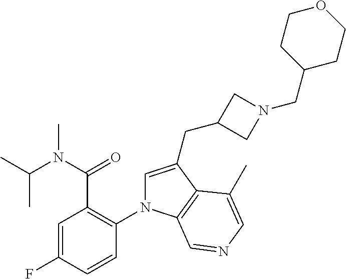 [SMILES: Cc1cncc2c1c(CC1CN(CC3CCOCC3)C1)cn2-c1ccc(F)cc1C(=O)N(C)C(C)C]

To a solution of intermediate 25 (0.082 g, 0.21 mmol) in 1,2-DCE (2.0 mL) was add tetrahydropyran-4-carbaldehyde (0.028 g, 0.25 mmol), and followed by NaBH(OAc)₃ (0.062 g, 0.29 mmol). After stirring at ambient temperature overnight, another portion of tetrahydropyran-4-carbaldehyde (0.028 g, 0.25 mmol) and NaBH(OAc)₃ (0.062 g, 0.29 mmol) was added. After stirring for another 1.5 h, 1M aq. NaOH solution was added, followed by DCM. The layers were separated, and the aqueous layer was extracted 4× with DCM. The organic layers were combined, dried over Na₂SO₄, filtered and evaporated. The residue was purified by RP-preparative HPLC (Stationary phase: RP Xbridge Prep C18 OBD-5 μm, 50×250 mm, Mobile phase: 0.5% NH₄HCO₃ solution in water, CH₃CN) to give Compound 61 (0.058 g, 57% yield), after lyophilization, as a white fluffy powder.

### Preparation of Compound 62:

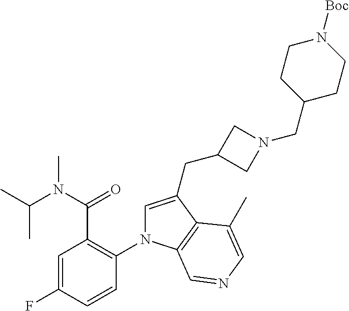 [SMILES: Cc1cncc2c1c(CC1CN(CC3CCN(C(=O)OC(C)(C)C)CC3)C1)cn2-c1ccc(F)cc1C(=O)N(C)C(C)C]

To a solution of intermediate 25 (0.18 g, 0.45 mmol) in 1,2-DCE (4.2 mL) was added N-Boc-piperidine-4-carboxaldehyde (0.11 g, 0.54 mmol), and followed by NaBH(OAc)₃ (0.13 g, 0.63 mmol). After stirring at ambient temperature overnight, DCM and 1M aq. NaOH were added. The layers were separated, and the aqueous layer was extracted 4×more with DCM. The organic layers were combined, dried over Na₂SO₄, filtered and evaporated. The residue was purified by silica gel column chromatography eluting with methanol (+1% 7N NH₃ in MeOH) in dichloromethane from 0% to 10% to give Compound 62 (0.25 g, 94% yield) as a foam.

### Preparation of Compound 63:

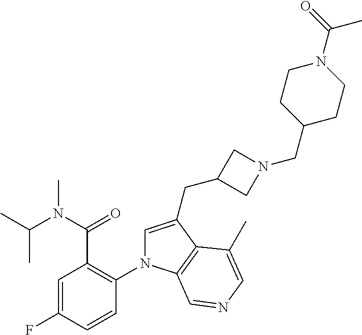 [SMILES: CC(=O)N1CCC(CN2CC(Cc3cn(-c4ccc(F)cc4C(=O)N(C)C(C)C)c4cncc(C)c34)C2)CC1]

To a solution of Compound 62 (0.25 g, 0.42 mmol) in DCM (5 mL) was added TFA (5 mL). After stirring at ambient temperature for 2 h, the reaction mixture was evaporated to dryness and the residue applied to SiliaBond® propylsulfonic acid resin as a solution in MeOH. The column was eluted with MeOH (8 fractions), followed by 3.5 N NH₃ in MeOH (8 fractions). Product containing fractions were pooled and evaporated to give an intermediate, which was dissolved in DCM (3.6 mL). The solution was cooled to 0° C. in an ice bath and DIPEA (0.13 mL, 0.76 mmol) was added, followed by Ac₂O (0.06 mL, 0.63 mmol). The resulting mixture was stirred at ambient temperature for 2 h, after which LC/MS showed full conversion of the starting material. Then, sat. aq. NaHCO₃ solution was added. The resulting mixture was partitioned between 1M aq. NaOH solution and DCM. The water layer was extracted 5× with DCM and the organic layers were combined, dried over Na₂SO₄, filtered, and evaporated to dryness. The residue was purified by RP-preparative HPLC (Stationary phase: RP Xbridge Prep C18 OBD-5 μm, 50×250 mm, Mobile phase: 0.5% NH₄HCO₃ solution in water, CH₃CN) to give Compound 63 (0.046 g, 68% yield) after lyophilization as a white fluffy powder.

The Following Compounds were Synthesized by an Analogous Method as Described for Compound 63

| Compound No. | Structure | Starting Materials |
|---|---|---|
| Compound 64 | 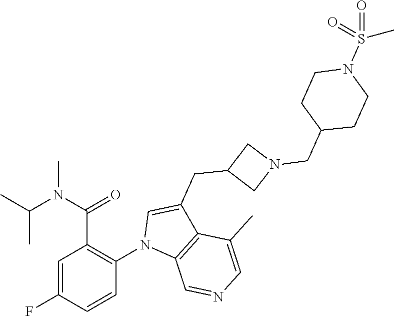 [SMILES: Cc1cncc2c1c(CC1CN(CC3CCN(S(C)(=O)=O)CC3)C1)cn2-c1ccc(F)cc1C(=O)N(C)C(C)C] | Compound 62 & methanesulfonyl chloride |
| Compound 65 | 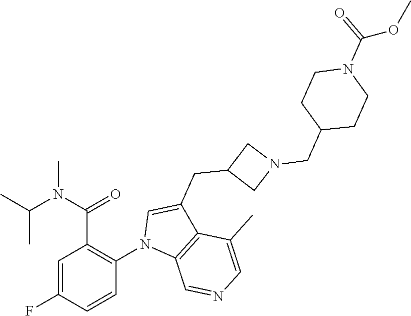 [SMILES: COC(=O)N1CCC(CN2CC(Cc3cn(-c4ccc(F)cc4C(=O)N(C)C(C)C)c4cncc(C)c34)C2)CC1] | Compound 62 & methyl carbonochloridate |

### Preparation of Compound 66:

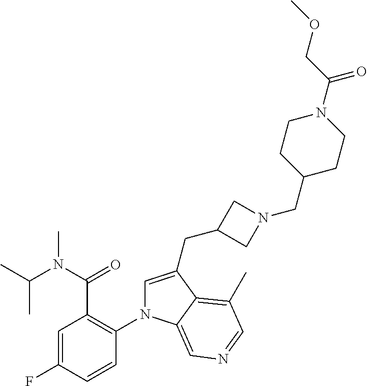 [SMILES: COCC(=O)N1CCC(CN2CC(Cc3cn(-c4ccc(F)cc4C(=O)N(C)C(C)C)c4cncc(C)c34)C2)CC1]

To a solution of Compound 62 (450 mg, 0.760 mmol) in anhydrous dichloromethane (4 mL) was added trifluoroacetic acid (4 mL). After stirring at 25° C. for 30 minutes, the reaction mixture was concentrated under reduced pressure to give a residue, which was diluted with dichloromethane (80 mL) and then basified to pH=14 with 10% aqueous NaOH (50 mL). The mixture was extracted with dichloromethane (60 mL×3) and the combined organic layers were dried over anhydrous Na₂SO₄, filtered and concentrated under reduced pressure to give the crude product (260 mg, crude) as a white solid, which was used for next step without further purification. To a solution of crude product (100 mg, 0.203 mmol) and 2-methoxyacetic acid (18.3 mg, 0.203 mmol) in anhydrous dichloromethane (3 mL) was added N,N-diisopropylethylamine (31.5 mg, 0.244 mmol). HATU (77.3 mg, 0.203 mmol) was added to the mixture under stirring, then the reaction mixture was stirred at 25° C. for 1 h. The reaction mixture was diluted with dichloromethane (20 mL), water (30 mL) was added. The mixture was extracted with dichloromethane (20 mL×3). The combined organic layers were dried over anhydrous Na₂SO₄, filtered and concentrated under reduced pressure to give a residue, which was purified by preparative-HPLC (Column: Phenomenex Gemini-NX C18 75*30 mm*3 um, Mobile Phase A: water (0.05% NH₃H₂O+10 mM NH₄HCO₃), Mobile Phase B: acetonitrile, Flow rate: 25 mL/min, gradient condition from 28% B to 58% B). The pure fractions were collected and the solvent was evaporated under vacuum. The residue was partitioned between acetonitrile (2 mL) and water (10 mL). The mixture was lyophilized to dryness to give Compound 66 (50.0 mg, 99.4% purity, 43.4% yield) as a white powder.

### The Following Compound was Synthesized by an Analogous Method as Described for Compound 66

| Compound No. | Structure | Starting Materials |
|---|---|---|
| Compound 67 | 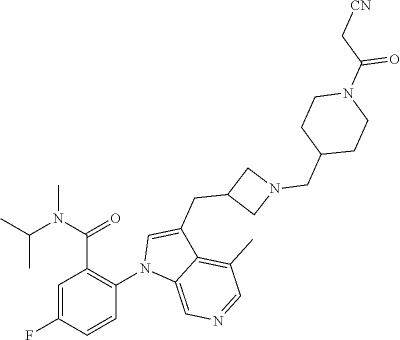 [SMILES: Cc1cncc2c1c(CC1CN(CC3CCN(C(=O)CC#N)CC3)C1)cn2-c1ccc(F)cc1C(=O)N(C)C(C)C] | Compound 62 & cyanoacetic acid |

### Preparation of Compound 68:

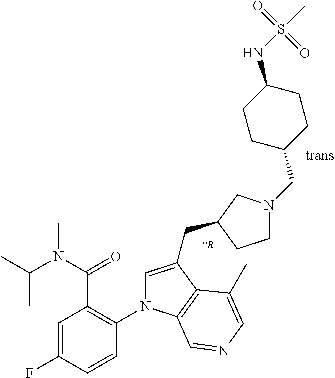

At 0° C., to a solution of Compound 488 (380 mg, 0.6 mmol) and triethylamine (500 mg, 4.94 mmol) in anhydrous dichloromethane (10 mL) was added methanesulfonyl chloride (500 mg, 4.37 mmol) dropwise. The reaction mixture was warmed up to r.t. and stirred for 2 hours. The reaction mixture was quenched with a saturated solution of sodium bicarbonate (20 mL) and H₂O (20 mL) and extracted with dichloromethane (30 mL×3). The combined organic layers were dried over anhydrous Na₂SO₄, filtered, and concentrated under reduced pressure to give the crude product, which was purified by preparative-HPLC (Column: Phenomenex Gemini-NX C18 75*30 mm*3 um, Mobile Phase A: water (0.04% NH₃H₂O+10 mM NH₄HCO₃), Mobile Phase B: acetonitrile, Flow rate: 25 mL/min, gradient condition from 33% B to 63% B). The pure fractions were collected and the solvent was evaporated under vacuum. The residue was partitioned between acetonitrile (2 mL) and water (10 mL). The mixture was lyophilized to dryness to give the desired compound (150 mg, 97.9% purity, 41% yield) as a white powder.

The Following Compounds were Synthesized by an Analogous Method as Described for Compound_68

| Compound No. | Structure | Starting Materials |
|---|---|---|
| Compound 69 | 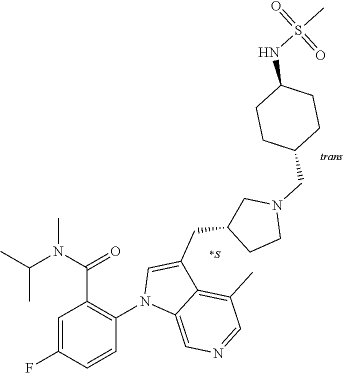 [SMILES: Cc1cncc2c1c(C[C@H]1CCN(C[C@H]3CC[C@H](NS(C)(=O)=O)CC3)C1)cn2-c1ccc(F)cc1C(=O)N(C)C(C)C] | Intermediate 27 & methanesulfonyl chloride |
| Compound 115 | 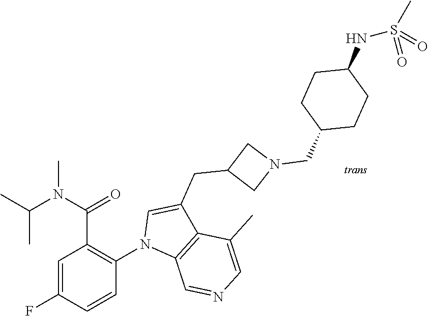 [SMILES: Cc1cncc2c1c(CC1CN(C[C@H]3CC[C@H](NS(C)(=O)=O)CC3)C1)cn2-c1ccc(F)cc1C(=O)N(C)C(C)C] | Compound 441 & methanesulfonyl chloride |

### Preparation of Compound 70:

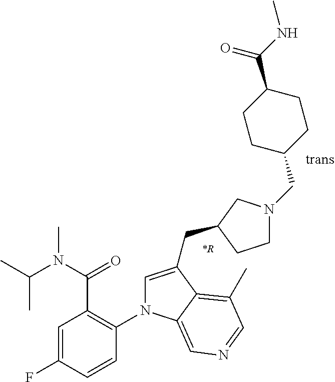

Intermediate 26 (75 mg, 0.184 mmol), DMF (4 mL), intermediate 116 (75 mg, 0.230 mmol), cesium carbonate (180 mg, 0.552 mmol) and potassium iodide (7 mg, 0.042 mmol) were added to a 50 mL round-bottomed flask. After degassing with N₂, the reaction mixture was heated and stirred at 100° C. overnight. The reaction mixture was poured into water (10 mL) and extracted with DCM (10 mL×3). The combined organic extracts were dried over anhydrous Na₂SO₄, filtered, and concentrated to dryness under reduced pressure to give the crude product which was purified by prep. HPLC (Phenomenex Gemini-NX C18 75*30 mm*3 um, Mobile Phase A: water (0.05% NH₃H₂O+10 mM NH₄HCO₃), Mobile Phase B: acetonitrile, Flow rate: 25 mL/min, gradient condition from 32% B to 62% B). The pure fractions were collected, and the solvent was evaporated under vacuum to give a residue. The residue was partitioned between acetonitrile (2 mL) and water (10 mL). The solution was lyophilized to dryness to give Compound 70 (34.27 mg, 98.3% purity, 33% yield) as a yellow solid.

### Preparation of Compound 71:

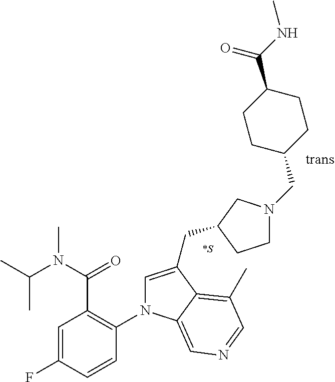

A mixture of intermediate 27 (75 mg, 0.184 mmol), intermediate 116 (75 mg, 0.230 mmol), cesium carbonate (180 mg, 0.552 mmol) and potassium iodide (7 mg, 0.042 mmol) in DMF (4 mL) was degassed with N₂ and the reaction mixture was heated and stirred at 100° C. overnight.

After cooled down to room temperature, the reaction mixture was poured into water (10 mL) and extracted with DCM (10 mL×3). The combined organic extracts were dried over Na₂SO₄, filtered, and concentrated to dryness under reduced pressure to give the crude product which was purified by prep. HPLC (Welch Xtimate C18 150*30 mm*5 um, Mobile Phase A: water (0.05% NH₃H₂O+10 mM NH₄HCO₃), Mobile Phase B: acetonitrile, Flow rate: 35 mL/min, gradient condition from 25% B to 55% B). The pure fractions were collected, and the solvent was evaporated under vacuum to give a residue. The residue was partitioned between acetonitrile (2 mL) and water (10 mL). The solution was lyophilized to dryness to give Compound 71 (33 mg, 96.1% purity, 30.7% yield) as a yellow solid.

### The Following Compound was Synthesized by an Analogous Method as Described for Compound 71

| Compound No. | Structure | Starting Materials |
|---|---|---|
| Compound 140 | 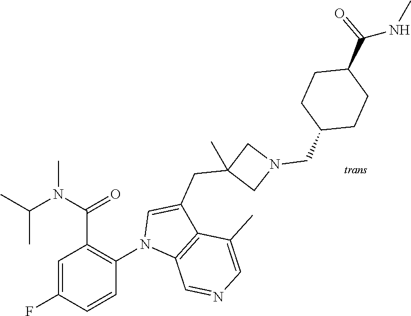 [SMILES: CNC(=O)[C@H]1CC[C@H](CN2CC(C)(Cc3cn(-c4ccc(F)cc4C(=O)N(C)C(C)C)c4cncc(C)c34)C2)CC1] | Compound 506 & intermediate 116 |

### Preparation of Compound 72:

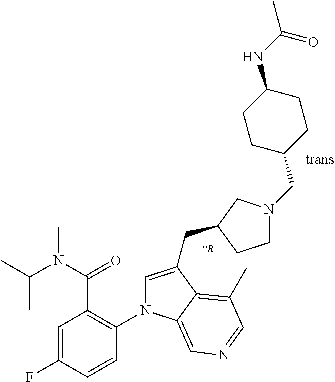

Triethylamine (80.0 mg, 0.791 mmol) was added to a solution of Compound 488 (80 mg, 0.126 mmol) in dichloromethane (3.0 mL). Then acetic anhydride (20.0 mg, 0.196 mmol) was added. After stirring at 25° C. for 30 minutes, the reaction mixture was suspended into aq. NaHCO₃ solution (30 mL) and extracted with dichloromethane (20 mL×2). The combined organic layers were dried over anhydrous Na₂SO₄, filtered and concentrated under reduced pressure to give a residue, which was purified by prep. HPLC (Column: Phenomenex Gemini-NX C18 75*30 mm*3 um, Mobile Phase A: water (0.05% NH₃H₂O+10 mM NH₄HCO₃), Mobile Phase B: acetonitrile, Flow rate: 25 mL/min, gradient condition from 46% B to 76% B). The pure fraction was collected and the solvent was evaporated under vacuum. The residue was re-suspended in water (10 mL) and the resulting mixtures were lyophilized to dryness to give Compound 72 (20.0 mg, 100% purity, 28.2% yield) as a white powder.

### The Following Compound was Synthesized by an Analogous Method as Described for Compound 72

| Compound No. | Structure | Starting Materials |
|---|---|---|
| Compound 73 | 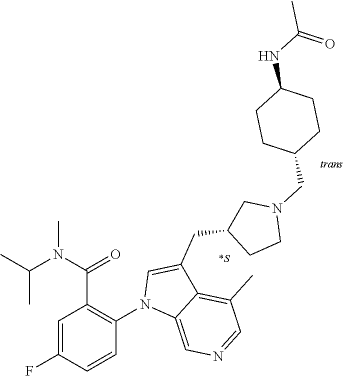 [SMILES: CC(=O)N[C@H]1CC[C@H](CN2CC[C@H](Cc3cn(-c4ccc(F)cc4C(=O)N(C)C(C)C)c4cncc(C)c34)C2)CC1] | Intermediate 27 |

### Preparation of Compound 74:

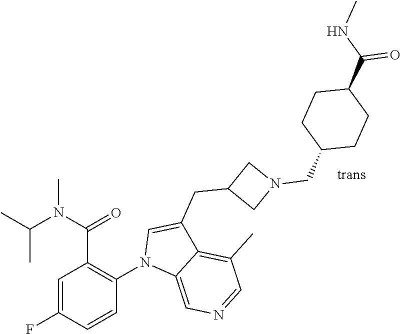 [SMILES: CNC(=O)[C@H]1CC[C@H](CN2CC(Cc3cn(-c4ccc(F)cc4C(=O)N(C)C(C)C)c4cncc(C)c34)C2)CC1]

Intermediate 25 (185 mg, 0.469 mmol), DMF (5 mL), intermediate 116 (185 mg, 0.568 mmol), cesium carbonate (460 mg, 1.41 mmol) and potassium iodide (16 mg, 0.096 mmol) were combined into a 50 mL round-bottomed flask. After degassing with N₂, the reaction mixture was heated and stirred at 100° C. for 6 hours. After cooled down to the room temperature, the reaction mixture was poured into water (30 mL) and extracted with EtOAc (30 mL×3). The combined organic extracts were dried over Na₂SO₄, filtered, and concentrated to dryness under reduced pressure to give the crude product which was purified by prep. HPLC (Phenomenex Gemini-NX C18 75*30 mm*3 um, Mobile Phase A: water (0.05% NH₃H₂O+10 mM NH₄HCO₃), Mobile Phase B: acetonitrile, Flow rate: 25 mL/min, gradient condition from 33% B to 63% B). The pure fractions were collected, and the solvent was evaporated under vacuum to give a residue. The residue was partitioned between acetonitrile (2 mL) and water (10 mL). The solution was lyophilized to dryness to give Compound 74 (40.78 mg, 95.7% purity, 15% yield) as a brown powder.

### Preparation of Compound 83:

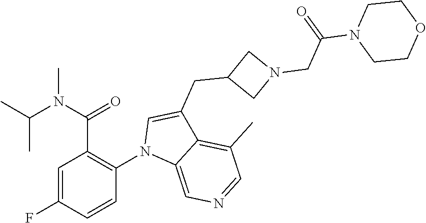 [SMILES: Cc1cncc2c1c(CC1CN(CC(=O)N3CCOCC3)C1)cn2-c1ccc(F)cc1C(=O)N(C)C(C)C]

Intermediate 25 (0.060 g, 0.152 mmol) was dissolved in MeCN (1.6 mL). Then, 4-(2-chloroacetyl)morpholine (0.027 g, 0.17 g) and triethylamine (0.13 mL, 0.91 mmol) was added and the resulting mixture stirred at ambient temperature for 2 h. Next, MeOH was added and the mixture was evaporated to dryness. The residue was purified by RP-preparative HPLC (Stationary phase: RP Xbridge Prep C18 OBD-5 μm, 50×250 mm, Mobile phase: 0.5% NH₄HCO₃ solution in water, CH₃CN) to give Compound 83 (32 mg, 0.059 mmol, 39% yield).

The Following Compounds were Synthesized by an Analogous Method as Described for Compound 83

| Compound No. | Structure | Starting Materials |
|---|---|---|
| Compound 96 | 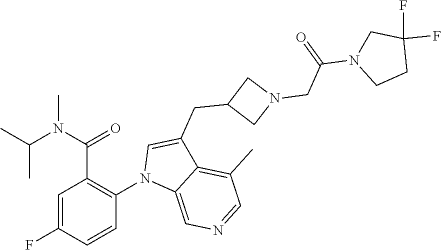 [SMILES: Cc1cncc2c1c(CC1CN(CC(=O)N3CCC(F)(F)C3)C1)cn2-c1ccc(F)cc1C(=O)N(C)C(C)C] | Intermediate 25 & 147 |
| Compound 97 | 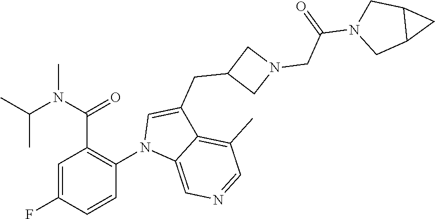 [SMILES: Cc1cncc2c1c(CC1CN(CC(=O)N3CC4CC4C3)C1)cn2-c1ccc(F)cc1C(=O)N(C)C(C)C] | Intermediate 25 & 148 |
| Compound 98 | 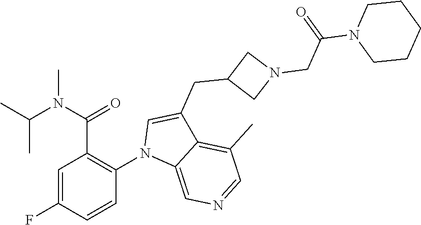 [SMILES: Cc1cncc2c1c(CC1CN(CC(=O)N3CCCCC3)C1)cn2-c1ccc(F)cc1C(=O)N(C)C(C)C] | Intermediate 25 & 2-chloro-1- piperidin-1-yl-ethanone |

### Preparation of Compound 75:

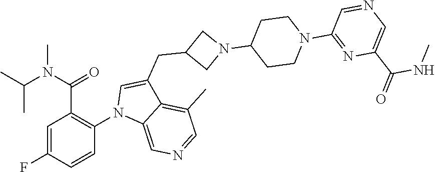 [SMILES: CNC(=O)c1cncc(N2CCC(N3CC(Cc4cn(-c5ccc(F)cc5C(=O)N(C)C(C)C)c5cncc(C)c45)C3)CC2)n1]

To a solution of intermediate 118 (0.060 g, 0.24 mmol) and intermediate 25 (0.11 g, 0.29 mmol) in MeOH (1 mL) was added AcOH (28 μL, 0.48 mmol), followed by NaBH₃CN (0.030 g, 0.48 mmol). The mixture was stirred at ambient temperature overnight. Next, sat. aq. NaHCO₃ solution was added. After stirring for −5 min, the mixture was evaporated to dryness. The residue was purified by preparative HPLC (Stationary phase: RP Xbridge Prep C18 OBD-5 μm, 50×250 mm, Mobile phase: 0.5% NH₄HCO₃ solution in water, CH₃CN) to give Compound 75 (0.042 g, 49% yield).

### Preparation of Compound 76:

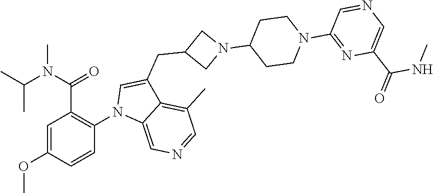 [SMILES: CNC(=O)c1cncc(N2CCC(N3CC(Cc4cn(-c5ccc(OC)cc5C(=O)N(C)C(C)C)c5cncc(C)c45)C3)CC2)n1]

To a solution of intermediate 118 (0.058 g, 0.23 mmol) and intermediate 121 (0.14 g, 0.28 mmol) in MeOH (1 mL) was added AcOH (27 μL, 0.47 mmol), followed by NaBH₃CN (0.029 g, 0.47 mmol). The mixture was stirred at ambient temperature overnight. Next, sat. aq. NaHCO₃ solution was added. After stirring for ˜5 min, the mixture was evaporated to dryness. The residue was purified by preparative HPLC (Stationary phase: RP Xbridge Prep C18 OBD-5 μm, 50×250 mm, Mobile phase: 0.5% NH₄HCO₃ solution in water, CH₃CN) to give the product (0.090 g, 0.13 mmol) with 92% purity determined via ¹H NMR integration. (˜5% on UV via SFC). Additional purification via prep. SFC (Stationary phase: Chiralcel Diacel IH 20×250 mm, Mobile phase: CO₂, EtOH+0.4 iPrNH₂) yielded pure Compound 76 (0.061 g, 0.098 mmol).

### Preparation of Compound 77, 77a, 77b, 77c, 77d:

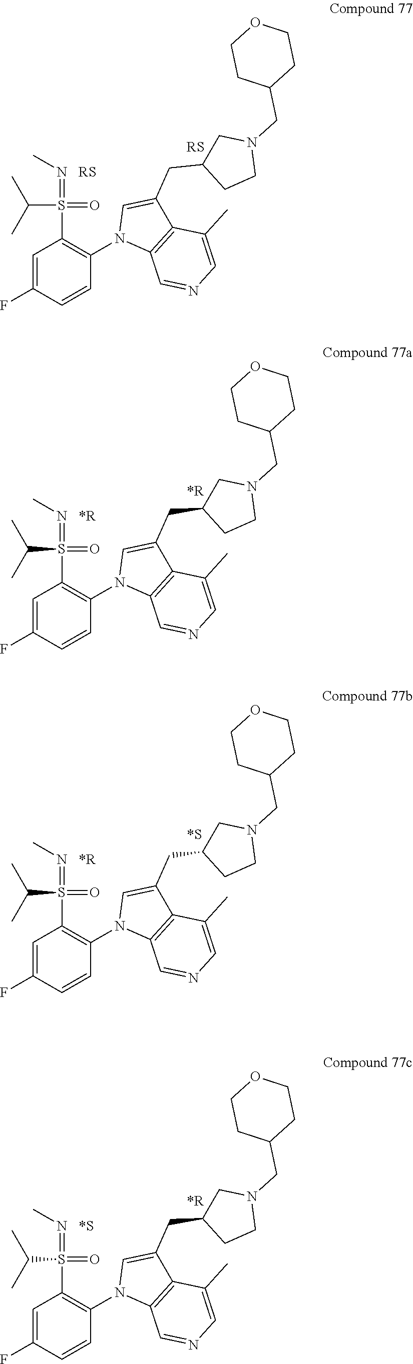 [SMILES: CN=S(=O)(c1cc(F)ccc1-n1cc(CC2CCN(CC3CCOCC3)C2)c2c(C)cncc21)C(C)C ; CN=[S@](=O)(c1cc(F)ccc1-n1cc(C[C@@H]2CCN(CC3CCOCC3)C2)c2c(C)cncc21)C(C)C ; CN=[S@](=O)(c1cc(F)ccc1-n1cc(C[C@H]2CCN(CC3CCOCC3)C2)c2c(C)cncc21)C(C)C ; CN=[S@@](=O)(c1cc(F)ccc1-n1cc(C[C@@H]2CCN(CC3CCOCC3)C2)c2c(C)cncc21)C(C)C]

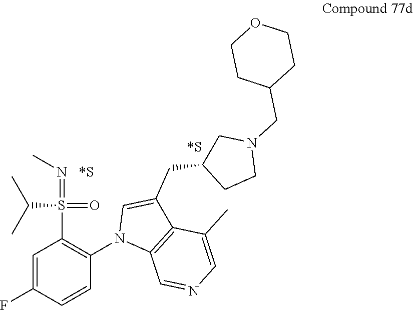 [SMILES: CN=[S@@](=O)(c1cc(F)ccc1-n1cc(C[C@H]2CCN(CC3CCOCC3)C2)c2c(C)cncc21)C(C)C]

A mixture of intermediate 130 (77.0 mg, 0.183 mmol) and tetrahydropyran-4-carbaldehyde (27.5 mg, 0.241 mmol) in MeOH (1.20 mL) was stirred for 30 min after which sodium cyanoborohydride (15.1 mg, 0.241 mmol) was added. The reaction mixture was stirred at rt overnight. The reaction was quenched with water and used as such for reversed-phase prep HPLC purification (Stationary phase: RP Xbridge Prep C18 OBD-5 μm, 50×250 mm, Mobile phase: 0.5% NH₄HCO₃ solution in water, CH₃CN), followed by purification by silica gel column chromatography eluting with ethyl acetate, followed by 20% methanol in dichloromethane to give Compound 77 (32 mg, 50.5% yield) as a white solid.

Compound 77 was further purified by Prep SFC (Stationary phase: Chiralpak Diacel AD 20×250 mm, Mobile phase: CO₂, iPrOH+0.4 iPrNH₂). The first fraction was collected at Compound 77a, the second fraction as Compound 77b, the third fraction as Compound 77c and the fourth fraction as Compound 77d.

The Following Compounds were Synthesized by an Analogous Method as Described for Compound 77

| Compound No. | Structure | Starting Materials |
|---|---|---|
| Compound 78 | 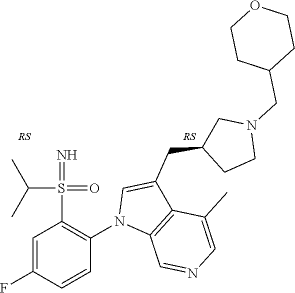 [SMILES: Cc1cncc2c1c(C[C@@H]1CCN(CC3CCOCC3)C1)cn2-c1ccc(F)cc1S(=N)(=O)C(C)C] | Intermediate 131 |
| Compound 78a | 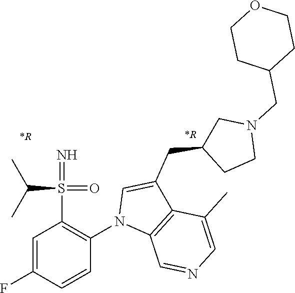 [SMILES: Cc1cncc2c1c(C[C@@H]1CCN(CC3CCOCC3)C1)cn2-c1ccc(F)cc1[S@](=N)(=O)C(C)C] | Compound 78 was purified by Prep SFC (Stationary phase: Chiralpak Diacel AD 20 × 250 mm, Mobile phase: CO₂, EtOH- iPrOH (50-50) + 0.4% iPrNH₂). The first fraction was collected as Compound 78a, the second fraction as Compound 78b, the third fraction as Compound 78c and the fourth fraction as Compound 78d |
| Compound 78b | 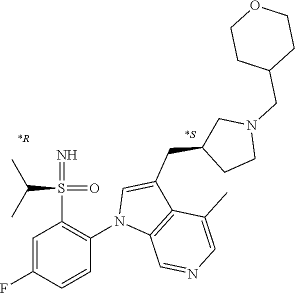 [SMILES: Cc1cncc2c1c(C[C@@H]1CCN(CC3CCOCC3)C1)cn2-c1ccc(F)cc1[S@](=N)(=O)C(C)C] | |
| Compound 78c & | 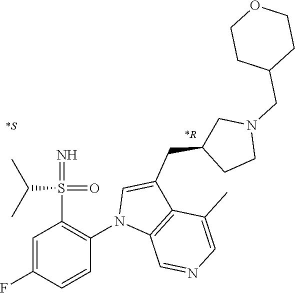 [SMILES: Cc1cncc2c1c(C[C@@H]1CCN(CC3CCOCC3)C1)cn2-c1ccc(F)cc1[S@@](=N)(=O)C(C)C] | |
| Compound 78d | 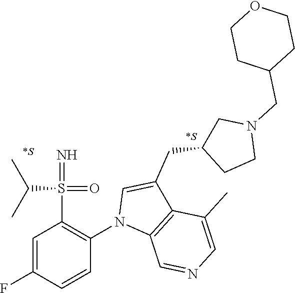 [SMILES: Cc1cncc2c1c(C[C@H]1CCN(CC3CCOCC3)C1)cn2-c1ccc(F)cc1[S@@](=N)(=O)C(C)C] | |
| Compound 79 | 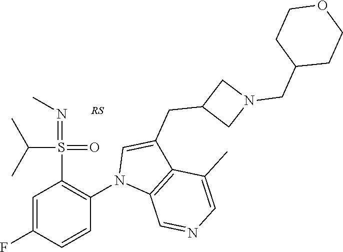 [SMILES: CN=S(=O)(c1cc(F)ccc1-n1cc(CC2CN(CC3CCOCC3)C2)c2c(C)cncc21)C(C)C] | Intermediate 1, tert-butyl 3- formylazetidine-1-carboxylate & intermediate 124 |
| Compound 80 | 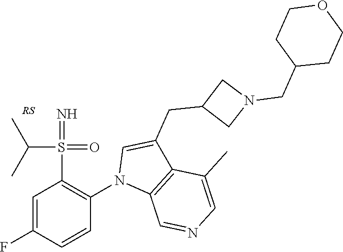 [SMILES: Cc1cncc2c1c(CC1CN(CC3CCOCC3)C1)cn2-c1ccc(F)cc1S(=N)(=O)C(C)C] | Intermediate 1, tert-butyl 3- formylazetidine-1-carboxylate & intermediate 125 |

### Preparation of Compound 84:

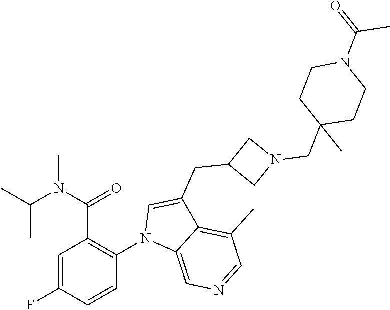 [SMILES: CC(=O)N1CCC(C)(CN2CC(Cc3cn(-c4ccc(F)cc4C(=O)N(C)C(C)C)c4cncc(C)c34)C2)CC1]

At 0° C., to a solution of Compound 430 (256 mg, 0.506 mmol) in dichloromethane (10 mL) were added acetyl chloride (40 mg, 0.510 mmol) and triethylamine (155 mg, 1.532 mmol). After stirring at r.t. for 1 hr, the mixture was quenched with saturated aqueous sodium hydrogen carbonate solution and extracted with dichloromethane three times. The combined organic layers were washed with brine, dried over anhydrous Na₂SO₄ and concentrated under vacuum. The residue was purified by prep. HPLC (Column: Waters Xbridge C18 OBD 5 μm, 19*150 mm, Mobile Phase A: water (0.1% NH₄HCO₃), Mobile Phase B: acetonitrile, Flow rate: 15 mL/min, gradient condition from 20% B to 60% B) to give Compound 84 (88 mg, 30.8% yield) as a white solid.

### Preparation of Compound 88:

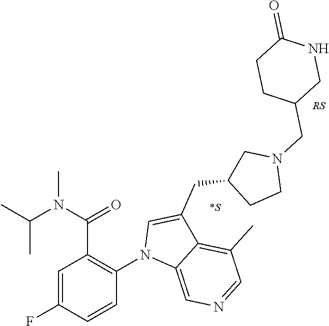 [SMILES: Cc1cncc2c1c(C[C@H]1CCN(CC3CCC(=O)NC3)C1)cn2-c1ccc(F)cc1C(=O)N(C)C(C)C]

Intermediate 27 (50 mg, 0.12 mmol), intermediate 134 (69.3 mg, 0.24 mmol), DIEA (0.105 mL, 0.61 mmol) and potassium iodide (20.3 mg, 0.12 mmol) were added to NMP (2 mL). The mixture was stirred at 70° C. for 16 hours. The mixture was separated by HPLC (Column: Waters Xbridge C18 5 μm, 19*150 mm, Mobile Phase A: water (0.1% NH₃H₂O+10 mM NH₄HCO₃), Mobile Phase B: acetonitrile, Flow rate: 17 mL/min, gradient condition from 20% B to 50% B). The pure fraction was collected and lyophilized to afford Compound 88 (20 mg, yield 29.80%).

The Following Compounds were Synthesized by an Analogous Method as Described for Compound 88

| Compound No. | Structure | Starting Materials |
|---|---|---|
| Compound 89 | 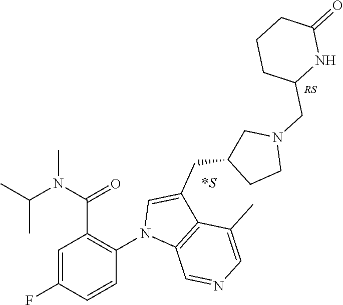 [SMILES: Cc1cncc2c1c(C[C@H]1CCN(CC3CCCC(=O)N3)C1)cn2-c1ccc(F)cc1C(=O)N(C)C(C)C] | Intermediate 27 & 135 |
| Compound 95 | 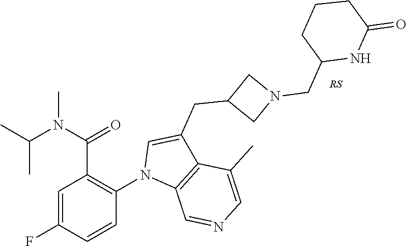 [SMILES: Cc1cncc2c1c(CC1CN(CC3CCCC(=O)N3)C1)cn2-c1ccc(F)cc1C(=O)N(C)C(C)C] | Intermediate 25 & 135 |
| Compound 105 | 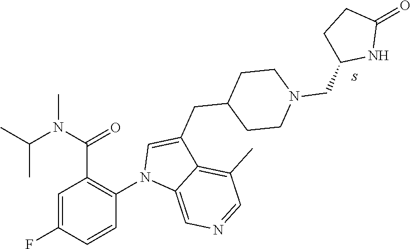 | Intermediate 28 & 159 |
| Compound 110 | 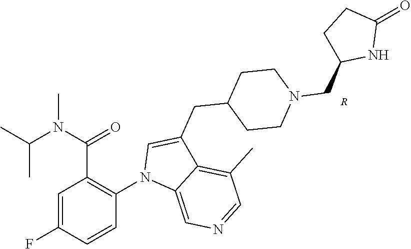 | Intermediate 28 & 162 |
| Compound 164a & Compound 164b | 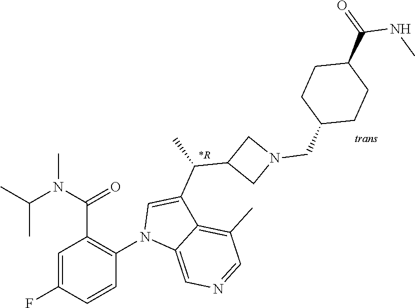 [SMILES: CNC(=O)[C@H]1CC[C@H](CN2CC([C@@H](C)c3cn(-c4ccc(F)cc4C(=O)N(C)C(C)C)c4cncc(C)c34)C2)CC1] | Intermediate 236 & 116 The product was separated by SFC (separation condition: DAICEL CHIRALPAK IG (250 mm* 30 mm, 10 um); Mobile phase: A: Supercritical CO₂, B: 0.1% NH₃H₂O MeOH, A:B = 55:45 at 80 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 164a and the second fraction as Compound 164b. |
| | 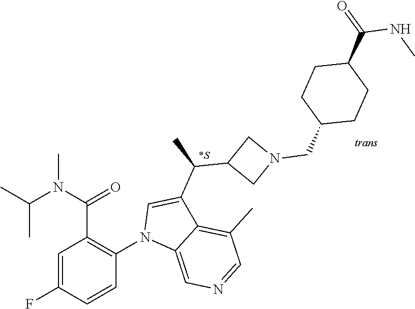 [SMILES: CNC(=O)[C@H]1CC[C@H](CN2CC([C@H](C)c3cn(-c4ccc(F)cc4C(=O)N(C)C(C)C)c4cncc(C)c34)C2)CC1] | |
| Compound 186a & Compound 186b | 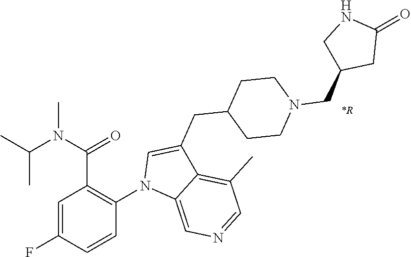 | Intermediate 28 & 249 The product was separated by SFC (Column ID: IH Column Size: 4.6 mm* 250 mm 5 um, Method: CAN-IPA-DEA-50- 50-0.3-30 MIN, Flow: 1 ml/min, Temperature: 30° C.). The first fraction was collected as Compound 186a and the second as Compound 186b. |
| | 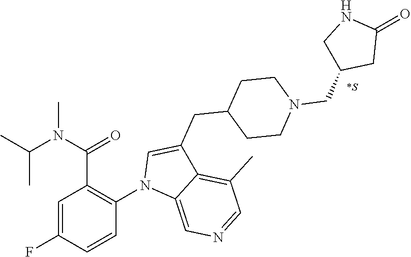 [SMILES: Cc1cncc2c1c(CC1CCN(C[C@@H]3CNC(=O)C3)CC1)cn2-c1ccc(F)cc1C(=O)N(C)C(C)C] | |
| Compound 195 | 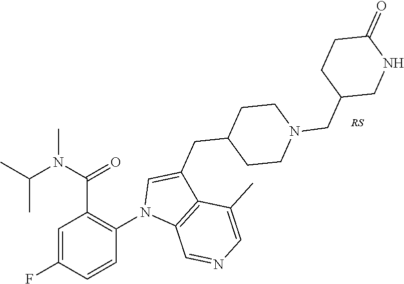 [SMILES: Cc1cncc2c1c(CC1CCN(CC3CCC(=O)NC3)CC1)cn2-c1ccc(F)cc1C(=O)N(C)C(C)C] | Intermediate 28 & 134 |
| Compound 212 | 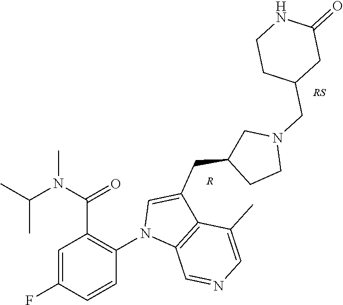 | Intermediate 202 & 273 |
| Compound 254 | 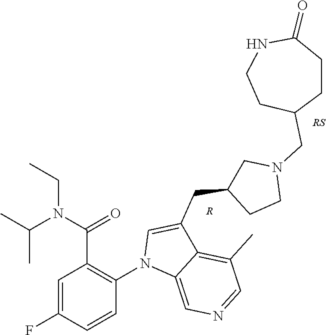 | Intermediate 336 & 338 |

### Preparation of Compound 91:

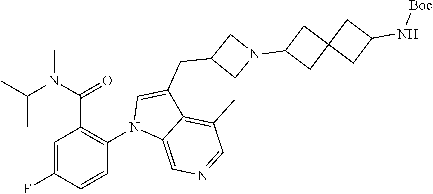 [SMILES: Cc1cncc2c1c(CC1CN(C3CC4(CC(NC(=O)OC(C)(C)C)C4)C3)C1)cn2-c1ccc(F)cc1C(=O)N(C)C(C)C]

A mixture of intermediate 25 (127 mg, 0.322 mmol), 2-(Boc-amino)-6-oxospiro[3.3]heptane (145 mg, 0.644 mmol) and AcOH (36.9 μL, 0.644 mmol) in MeOH (3.2 mL) was stirred for 30 min after which sodium cyanoborohydride (40.5 mg, 0.644 mmol) was added. The reaction mixture was stirred at 50° C. overnight. The reaction was cooled down to r.t., quenched with water, and evaporated to dryness. The residue was purified by silica gel column chromatography eluting with methanol (+1% NH₃ in MeOH) in dichloromethane from 1% to 50%. The purest fractions were collected, evaporated to dryness to afford Compound 91 (64 mg, yield 32.6%) as a white solid.

The Following Compounds were Synthesized by an Analogous Method as Described for Compound 91

| Compound No. | Structure | Starting Materials |
|---|---|---|
| Compound 92 | 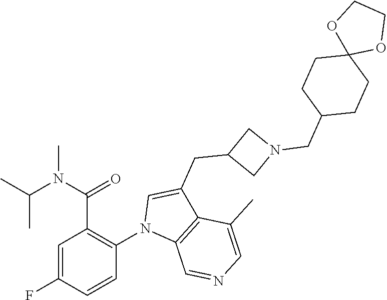 [SMILES: Cc1cncc2c1c(CC1CN(CC3CCC4(CC3)OCCO4)C1)cn2-c1ccc(F)cc1C(=O)N(C)C(C)C] | Intermediate 25 & 1,4- dioxaspiro[4.5]decane-8- carbaldehyde |
| Compound 104 | 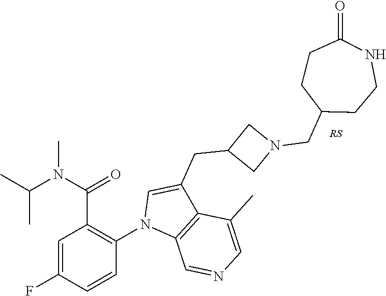 [SMILES: Cc1cncc2c1c(CC1CN(CC3CCNC(=O)CC3)C1)cn2-c1ccc(F)cc1C(=O)N(C)C(C)C] | Intermediate 25 & 7- oxoazepane-4-carbaldehyde |
| Compound 114 | 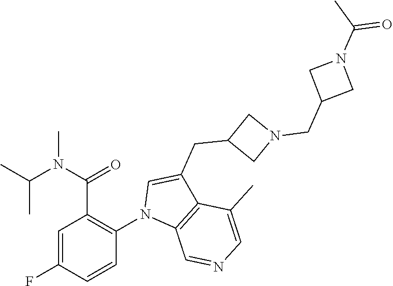 [SMILES: CC(=O)N1CC(CN2CC(Cc3cn(-c4ccc(F)cc4C(=O)N(C)C(C)C)c4cncc(C)c34)C2)C1] | Intermediate 25 & 1- acetylazetidine-3-carbaldehyde |
| Compound 120 | 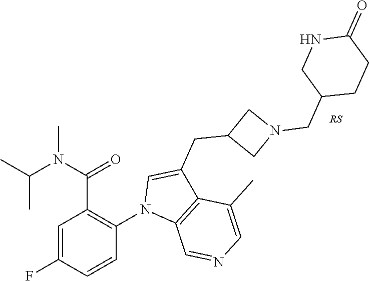 [SMILES: Cc1cncc2c1c(CC1CN(CC3CCC(=O)NC3)C1)cn2-c1ccc(F)cc1C(=O)N(C)C(C)C] | Intermediate 25 & 6- oxopiperidine-3-carbaldehyde |

### Preparation of Compound 93:

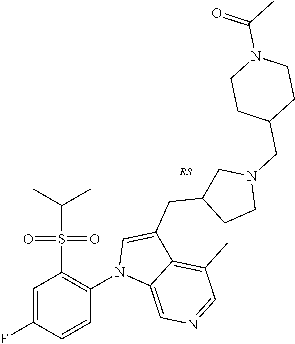 [SMILES: CC(=O)N1CCC(CN2CCC(Cc3cn(-c4ccc(F)cc4S(=O)(=O)C(C)C)c4cncc(C)c34)C2)CC1]

Compound 490 (55 mg, 0.107 mmol) was dissolved in DCM (1.2 mL). Then, DIPEA (0.11 mL, 0.64 mmol) was added, followed by Ac₂O (0.051 mL, 0.536 mmol). The resulting mixture was then stirred at ambient temperature for 2 h. Next, a small amount of MeOH was added, and the mixture evaporated to dryness. The compound was purified by silica gel column chromatography eluting with methanol (+1% 7N NH3 in MeOH) in dichloromethane from 1% to 20% to afford the product (80 mg), which was triturated with DEE to give Compound 93 (52.3 mg, yield 83.5%).
The Following Compounds were Synthesized by an Analogous Method as Described for Compound 93

| Compound No. | Structure | Starting Materials |
|---|---|---|
| Compound 94 | 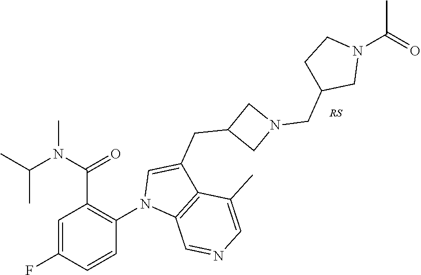 [SMILES: CC(=O)N1CCC(CN2CC(Cc3cn(-c4ccc(F)cc4C(=O)N(C)C(C)C)c4cncc(C)c34)C2)C1] | Compound 432 |
| Compound 99 | 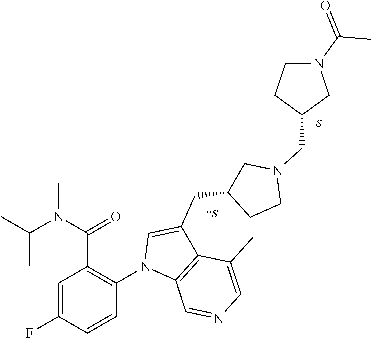 | Compound 434 |
| Compound 100 | 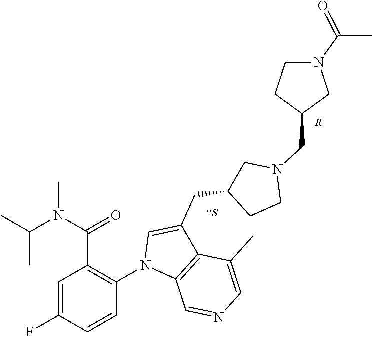 | Compound 435 |
| Compound 103 | 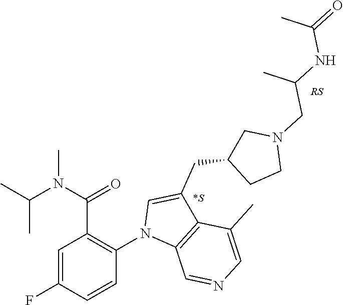 | Compound 437 |
| Compound 107 | 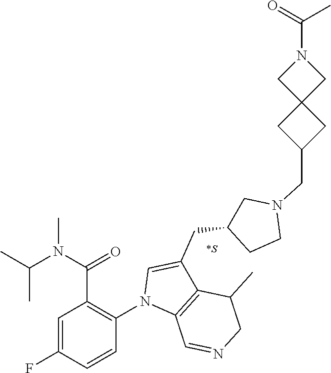 | Compound 438 |
| Compound 112 | 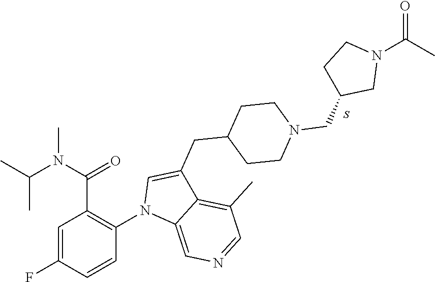 | Compound 439 |
| Compound 113 | 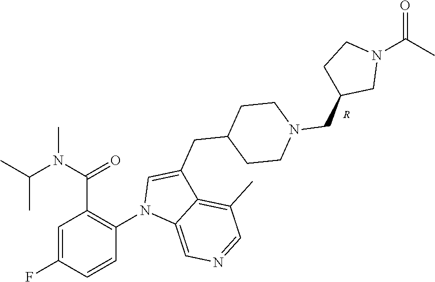 | Compound 502 |
| Compound 116a | 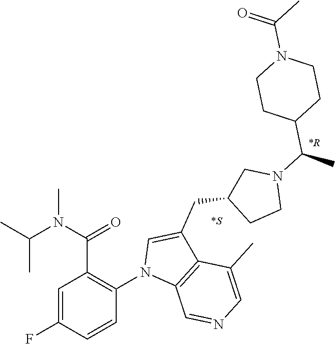 | Compound 442 |
| Compound 116b | 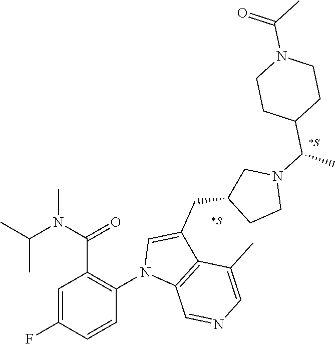 | Compound 443 |
| Compound 118 | 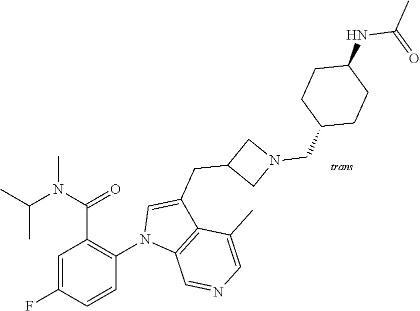 [SMILES: CC(=O)N[C@H]1CC[C@H](CN2CC(Cc3cn(-c4ccc(F)cc4C(=O)N(C)C(C)C)c4cncc(C)c34)C2)CC1] | Compound 441 |
| Compound 119a & Compound 119b | 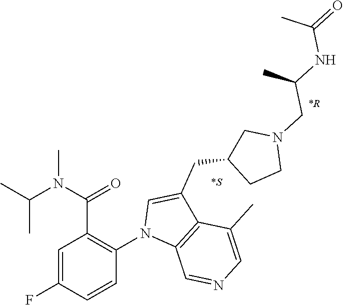 | Compound 444 The mixture was separated by SFC (separation condition: DAICEL CHIRALPAK AS (250 mm*30 mm, 10 um)); Mobile phase: A: Supercritical CO₂, B: 0.1% NH₃H₂O EtOH, A:B = 85:15 at 60 mL/min; Column Temp: 38 °C.; Nozzle Pressure: 100 Bar; Nozzle Temp: 60° C.; Evaporator Temp: 20° C.; Trimmer Temp: 25° C.; Wavelength: 220 nm). The first fraction was collected as Compound 119a and the second fraction as Compound 119b |
| | 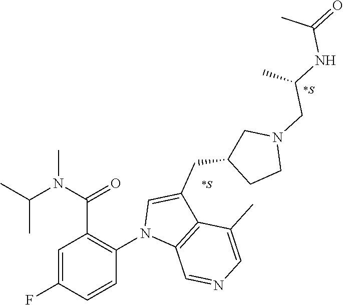 [SMILES: CC(=O)N[C@@H](C)CN1CC[C@H](Cc2cn(-c3ccc(F)cc3C(=O)N(C)C(C)C)c3cncc(C)c23)C1] | |
| Compound 127 | 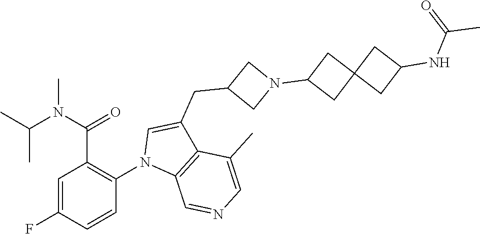 [SMILES: CC(=O)NC1CC2(C1)CC(N1CC(Cc3cn(-c4ccc(F)cc4C(=O)N(C)C(C)C)c4cncc(C)c34)C1)C2] | Compound 446 |
| Compound 137a & Compound 137b | 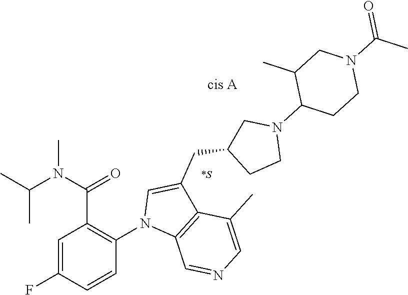 [SMILES: CC(=O)N1CCC(N2CC[C@H](Cc3cn(-c4ccc(F)cc4C(=O)N(C)C(C)C)c4cncc(C)c34)C2)C(C)C1] | Compound 391 The mixture was purified by the Prep. HPLC (Column: SunFire C18 150*19 mm*5 um, Mobile Phase A: water (0.1% NH₄Oac), Mobile Phase B: acetonitrile, Flow rate: 15 mL/min, gradient condition from 10% B to 50% B). The first fraction was collected as Compound 137a & the second fraction as Compound 137b. The absolute stereochemistry was not determined. |
| | 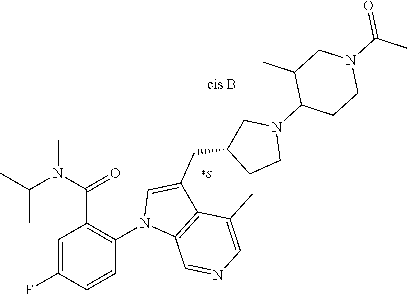 [SMILES: CC(=O)N1CCC(N2CC[C@H](Cc3cn(-c4ccc(F)cc4C(=O)N(C)C(C)C)c4cncc(C)c34)C2)C(C)C1] | |
| Compound 140 | 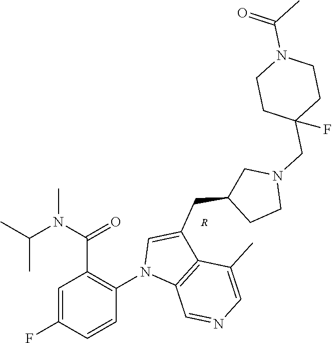 | Compound 447 |
| Compound 150 | 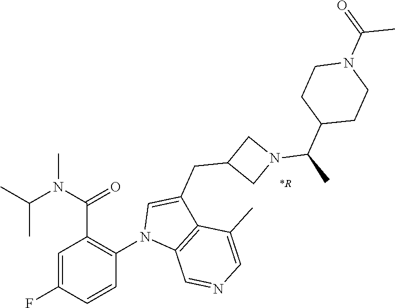 [SMILES: CC(=O)N1CCC([C@@H](C)N2CC(Cc3cn(-c4ccc(F)cc4C(=O)N(C)C(C)C)c4cncc(C)c34)C2)CC1] | Compound 449 |
| Compound 151 | 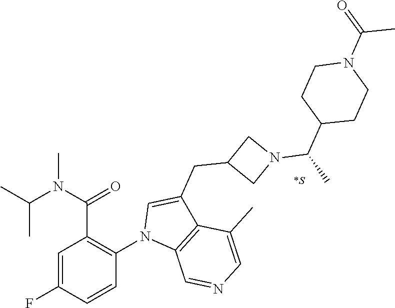 [SMILES: CC(=O)N1CCC([C@H](C)N2CC(Cc3cn(-c4ccc(F)cc4C(=O)N(C)C(C)C)c4cncc(C)c34)C2)CC1] | Compound 450 |
| Compound 175 | 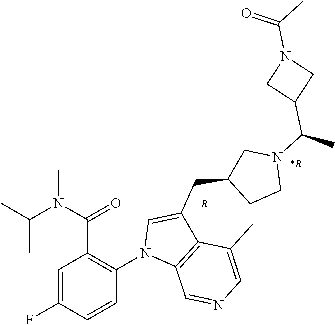 | Compound 452 |
| Compound 176 | 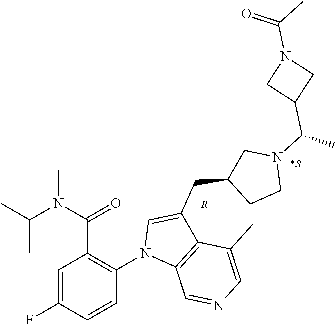 | Compound 453 |
| Compound 189 | 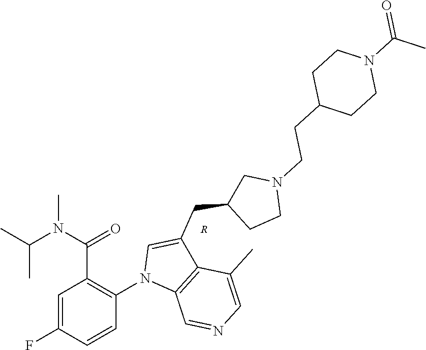 | Compound 456 |
| Compound 192 | 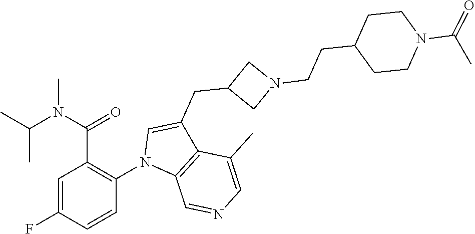 [SMILES: CC(=O)N1CCC(CCN2CC(Cc3cn(-c4ccc(F)cc4C(=O)N(C)C(C)C)c4cncc(C)c34)C2)CC1] | Compound 457 |
| Compound 215 | 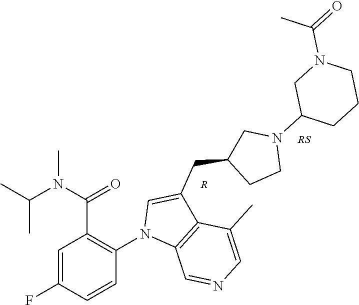 | Compound 466 |
| Compound 242 | 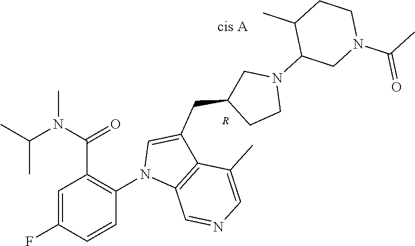 | Compound 475 |
| Compound 243 | 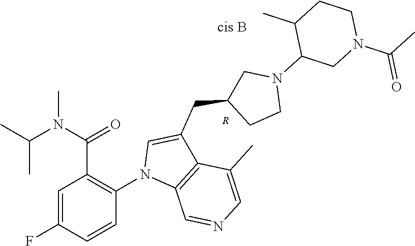 | Compound 476 |
| Compound 246 | 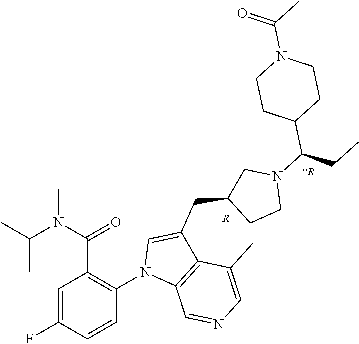 | Compound 477 |
| Compound 247 | 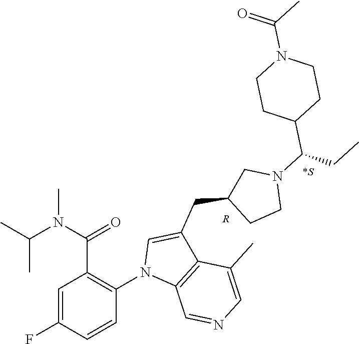 | Compound 478 |

### Preparation of Compound 101:

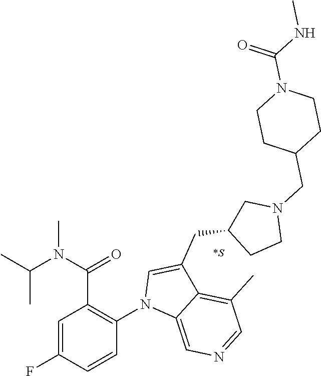 [SMILES: CNC(=O)N1CCC(CN2CC[C@H](Cc3cn(-c4ccc(F)cc4C(=O)N(C)C(C)C)c4cncc(C)c34)C2)CC1]

To a solution of Compound 485 (100 mg, 0.20 mmol), triethylamine (61 mg, 0.59 mmol) in dichloromethane (10 mL) was added a solution of methylaminoformyl chloride (23 mg, 0.22 mmol) in 2 mL of DCM. After stirring at 20° C. for 5 hr, the mixture was diluted with water (20 mL) and extracted with DCM (10 mL) for three times. The combined organic layers were washed with brine (30 mL), dried over Na₂SO₄ and filtered. The filtrate was concentrated under vacuum, which was purified by Prep-HPLC (Prep HPLC (Column: Xbridge C18 (5 m 19*150 mm), Mobile Phase A: Water (0.1% NH₄HCO₃), Mobile Phase B: acetonitrile, UV: 214 nm, Flow rate: 15 mL/min, Gradient: 15% B to 55% B) to give Compound 101 (90 mg, 0.15 mmol, 76.8% yield) as a white solid.

The Following Compounds were Synthesized by an Analogous Method as Described for Compound 101

| Compound No. | Structure | Starting Materials |
|---|---|---|
| Compound 168 | 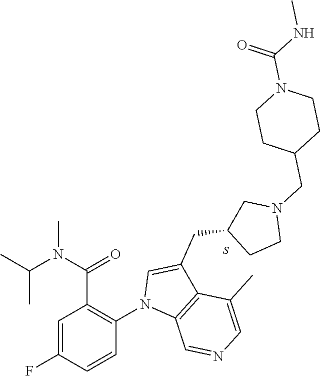 | Compound 451 & methylcarbamic chloride |
| Compound 197 | 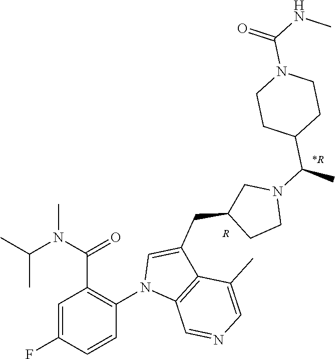 | Compound 458 & methylcarbamic chloride |
| Compound 202 | 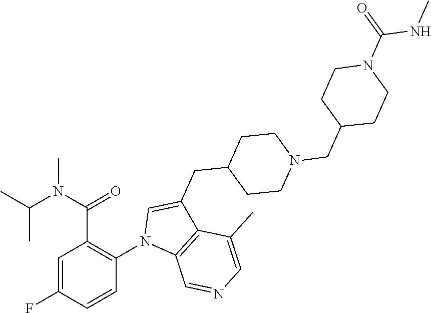 [SMILES: CNC(=O)N1CCC(CN2CCC(Cc3cn(-c4ccc(F)cc4C(=O)N(C)C(C)C)c4cncc(C)c34)CC2)CC1] | Compound 433 & methylcarbamic chloride |

### Preparation of Compound 129:

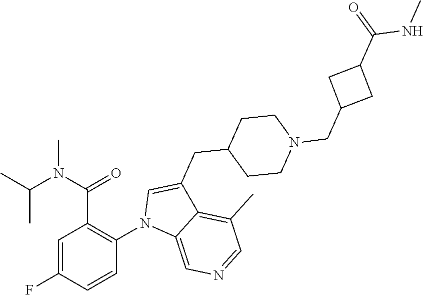 [SMILES: CNC(=O)C1CC(CN2CCC(Cc3cn(-c4ccc(F)cc4C(=O)N(C)C(C)C)c4cncc(C)c34)CC2)C1]

A mixture of intermediate 179 (85 mg, 0.16 mmol), 2 M methanamine in tetrahydrofuran (0.16 mL, 0.32 mmol), HATU (90 mg, 0.24 mmol, 1.5 equivalent), triethylamine (48 mg, 0.48 mmol, 3.0 equivalent) and DMF (10 mL) was stirred at room temperature overnight. The mixture was diluted with water (20 mL) and extracted with ethyl acetate (25 mL) for three times. The combined organic layers were dried over Na₂SO₄, filtered and concentrated to give the crude product, which was purified by prep. TLC eluting with 10% methanol in dichloromethane to give Compound 129 (55.3 mg, yield: 61.7%).

### Preparation of Compound 134, 134a, 134b, 134c, 134d:

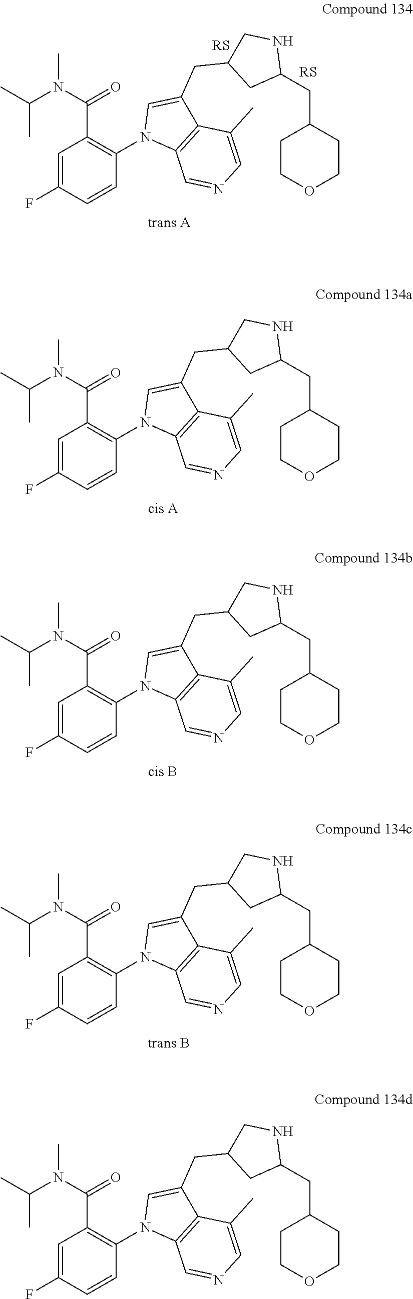 [SMILES: Cc1cncc2c1c(CC1CNC(CC3CCOCC3)C1)cn2-c1ccc(F)cc1C(=O)N(C)C(C)C ; Cc1cncc2c1c(CC1CNC(CC3CCOCC3)C1)cn2-c1ccc(F)cc1C(=O)N(C)C(C)C ; Cc1cncc2c1c(CC1CNC(CC3CCOCC3)C1)cn2-c1ccc(F)cc1C(=O)N(C)C(C)C ; Cc1cncc2c1c(CC1CNC(CC3CCOCC3)C1)cn2-c1ccc(F)cc1C(=O)N(C)C(C)C ; Cc1cncc2c1c(CC1CNC(CC3CCOCC3)C1)cn2-c1ccc(F)cc1C(=O)N(C)C(C)C]

TFA (1.39 mL, 18.13 mmol) was added to a solution of intermediate 199 (550 mg, 0.906 mmol) in DCM (10 mL) and stirred at rt for 3 h. The reaction mixture was concentrated under reduced pressure to give the TFA salt. TFA removal was done using SiliaBond@ propylsulfonic acid resin. The product was dissolved in MeOH and transferred to a column loaded with SiliaBond® propylsulfonic acid resin. The column was first eluted with MeOH after which the product was released by elution with ammoniated methanol (7 N). Tubes containing the product were concentrated under reduced pressure. The crude product was purified by silica gel column chromatography eluting with methanol (+1% 7N NH₃ in methanol) in dichloromethane from 0% to 10% to give Compound 134 (350 mg, yield 76%). Compound 134 was further separated via Prep. SFC (Stationary phase: Chiralpak Diacel AD 20×250 mm, Mobile phase: CO₂, EtOH+0.4 iPrNH₂) and Prep. SFC (Stationary phase: Chiralcel Diacel IH 20×250 mm, Mobile phase: CO₂, iPrOH+0.4 iPrNH₂) to afford Compound 134a (25 mg, 5.4% yield), Compound 134b (95 mg, 21% yield), Compound 134c (115 mg, 25% yield) & Compound 134d (36 mg, 7.7% yield).

### Preparation of Compound 142:

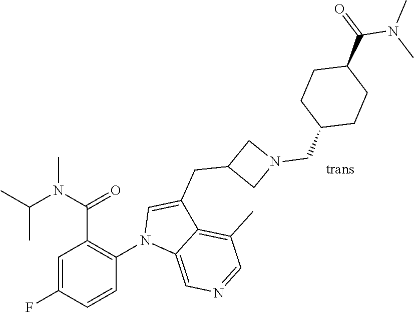 [SMILES: Cc1cncc2c1c(CC1CN(C[C@H]3CC[C@H](C(=O)N(C)C)CC3)C1)cn2-c1ccc(F)cc1C(=O)N(C)C(C)C]

A stir bar, intermediate 209 (50 mg, 0.086), EDCI (22 mg, 0.115 mmol), HOBt (21 mg, 0.114 mmol), DIEA (60 mg, 0.464 mmol), DCM (1 mL) and dimethylamine hydrochloride (16 mg, 0.196 mmol) were added into a 8 mL glass. The resulting mixture was stirred at room temperature for 4 h. The reaction mixture was poured it into water (5 m&), separated the layers, and the aqueous layers was extracted with DCM (5 mL×2). The combined organic extracts were dried over anhydrous Na₂S₄, filtered, and concentrated to dryness under reduced pressure to give the crude product which was purified by prep. HPLC (Column: Welch Xtimate C18 150*30 mm*5 um, Mobile Phase A: water (NH₃H₂O+NH₄HCO₃), Mobile Phase B: acetonitrile, Flow rate: 35 mE/min, gradient condition from 43% B to 73% B). The pure fractions were collected and the solvent was evaporated under vacuum to give a residue. The residue was partitioned between acetonitrile (2 mE) and water (10 mL). The solution was lyophilized to dryness to give Compound 142 (19 mg, 38.8% yield) as white power.

The Following Compounds were Synthesized by an Analogous Method as Described for Compound 142

Alternatively, purification can also be performed using the following method: prep-HPLC (Column: Welch Xtimate C18 150*25 mm*5 um, Mobile Phase A: water (+HCOOH), Mobile Phase B: acetonitrile Flow rate: 25 mL/min gradient condition from 2% B to 32% B).

| Compound No. | Structure | Starting Materials |
|---|---|---|
| Compound 143 | 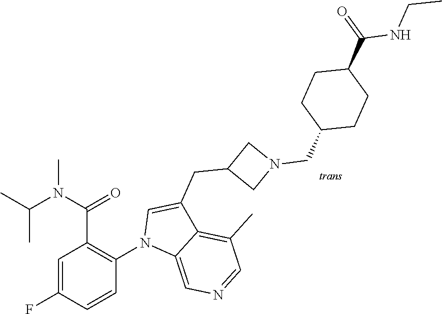 [SMILES: CCNC(=O)[C@H]1CC[C@H](CN2CC(Cc3cn(-c4ccc(F)cc4C(=O)N(C)C(C)C)c4cncc(C)c34)C2)CC1] | Intermediate 209 & ethanamine hydrochloride |
| Compound 155 | 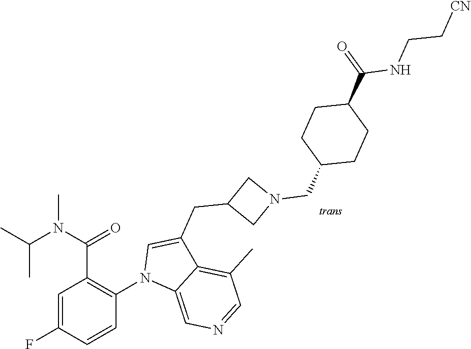 [SMILES: Cc1cncc2c1c(CC1CN(C[C@H]3CC[C@H](C(=O)NCCC#N)CC3)C1)cn2-c1ccc(F)cc1C(=O)N(C)C(C)C] | Intermediate 209 & 3- aminopropanenitrile |
| Compound 158 | 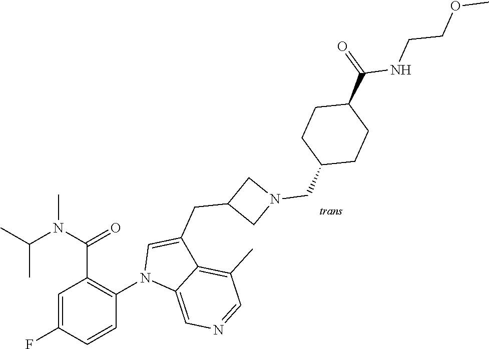 [SMILES: COCCNC(=O)[C@H]1CC[C@H](CN2CC(Cc3cn(-c4ccc(F)cc4C(=O)N(C)C(C)C)c4cncc(C)c34)C2)CC1] | Intermediate 209 & 2- methoxyethanamine |
| Compound 159 | 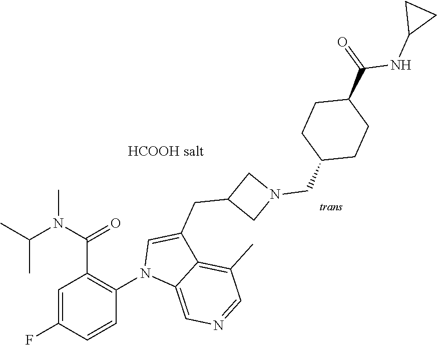 [SMILES: Cc1cncc2c1c(CC1CN(C[C@H]3CC[C@H](C(=O)NC4CC4)CC3)C1)cn2-c1ccc(F)cc1C(=O)N(C)C(C)C] | Intermediate 209 & cyclopropanamine |
| Compound 178 | 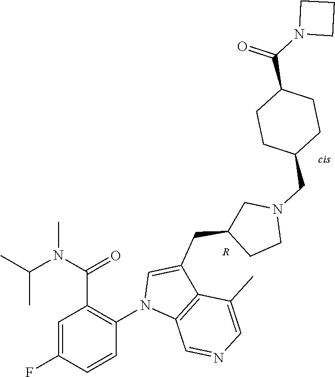 | Intermediate 247 & azetidine hydrochloride |
| Compound 179 | 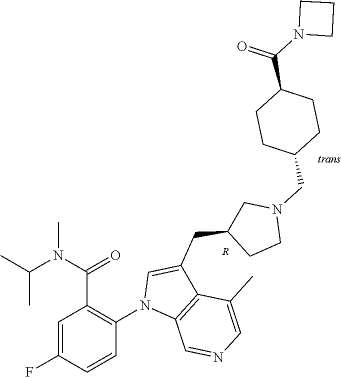 | Intermediate 248 & azetidine hydrochloride |
| Compound 185 | 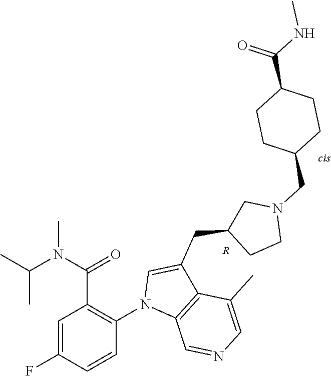 | Intermediate 247 & methylamine hydrochloride |
| Compound 221 | 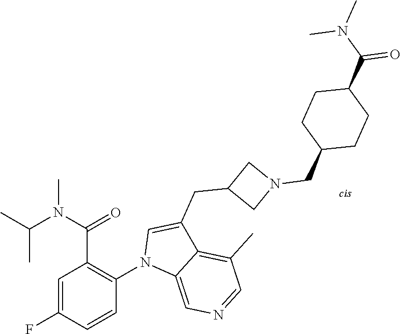 [SMILES: Cc1cncc2c1c(CC1CN(C[C@H]3CC[C@@H](C(=O)N(C)C)CC3)C1)cn2-c1ccc(F)cc1C(=O)N(C)C(C)C] | intermediate 292 & dimethylamine hydrochloride |
| Compound 236 | 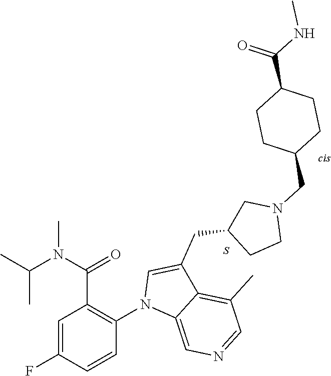 | Intermediate 316 & methanamine hydrochloride |
| Compound 241 | 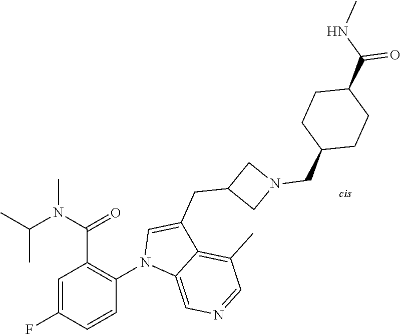 [SMILES: CNC(=O)[C@H]1CC[C@@H](CN2CC(Cc3cn(-c4ccc(F)cc4C(=O)N(C)C(C)C)c4cncc(C)c34)C2)CC1] | Intermediate 292 & methanamine hydrochloride |

### Preparation of Compound 144:

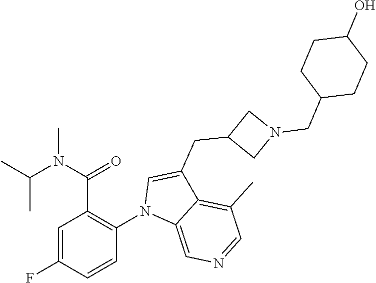 [SMILES: Cc1cncc2c1c(CC1CN(CC3CCC(O)CC3)C1)cn2-c1ccc(F)cc1C(=O)N(C)C(C)C]

Intermediate 211 (35.8 mg, 0.0582 mmol) and AcOH (6.66 μL, 0.116 mmol) were stirred in MeOH (0.581 mL) at rt for 30 min. Sodium cyanoborohydride (7.3 mg, 0.116 mmol) was added and the reaction was heated at 50° C. The reaction mixture was quenched with water and used as such for reversed-phase prep HPLC purification (Stationary phase: RP XBridge Prep C18 OBD-5 μm, 50×250 mm, Mobile phase: 0.5% NH₄HCO₃ solution in water, CH₃CN) to give Compound 144 (13.2 mg, 44.8% yield) as a white solid.

### Preparation of Compound 148:

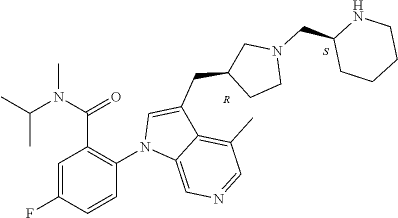

To a solution of intermediate 202 (60 mg, 0.14 mmol) in acetonitrile (5 mL) was added intermediate 216 (354 mg, 0.21 mmol), potassium carbonate (59 mg, 0.42 mmol) and potassium iodide (14 mg, 0.09 mmol). The reaction mixture was heated at 80° C. for 16 h. The mixture was cooled to room temperature, diluted with EtOAc and filtered. The filtrate was concentrated and purified by silica gel column chromatography eluting with 6% methanol in dichloromethane and prep. HPLC (Column: Xbridge C18 (5 μm 19*150 mm), Mobile Phase A: Water (0.1% ammonium bicarbonate), Mobile Phase B: acetonitrile, UV: 214 nm, Flow rate: 15 mL/min, Gradient: 15% B to 75% % B).

### The Following Compound was Synthesized by an Analogous Method as Described for Compound 148

The product obtained from the alkylation step was immediately treated with 7M HCl in ethyl acetate.

| Compound No. | Structure | Starting Materials |
|---|---|---|
| Compound 199 | 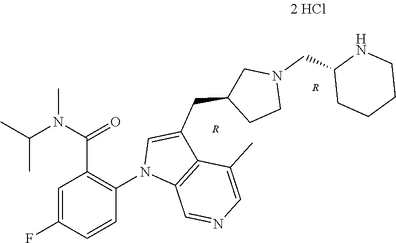 | Intermediate 202 & 260 |

### Preparation of Compound 152:

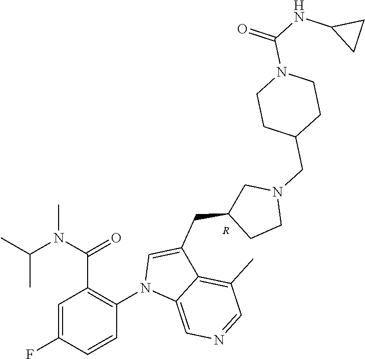

To a solution of Compound 448 (80 mg, 0.152 mmol) in tetrahydrofuran (4 mL) was added intermediate 221 (55 mg, 0.304 mmol). The reaction mixture was heated to 80° C. and stirred at this temperature overnight. The resulting mixture was concentrated and the residue was purified by silica gel column chromatography eluting with methanol in dichloromethane from 0% to 10% to give Compound 152 (65 mg, 70.6% yield) as a white solid.

### The Following Compound was Synthesized by an Analogous Method as Described for Compound 152

| Compound No. | Structure | Starting Materials |
|---|---|---|
| Compound 157 | 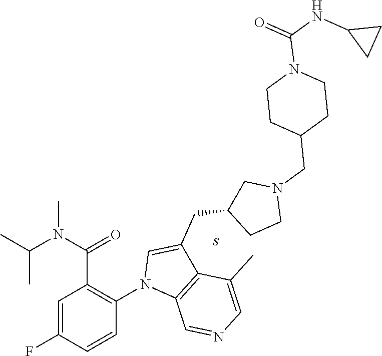 | Compound 451 & 221 |

### Preparation of Compound 153:

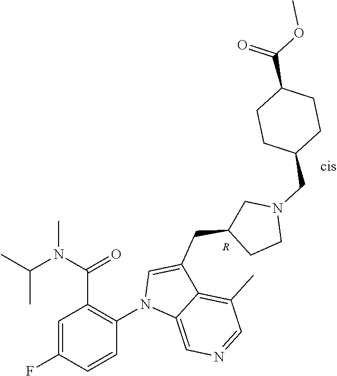

Intermediate 222 (50 mg, 0.2 mmol) was added to a stirred mixture of intermediate 202 (81.6 mg, 0.2 mmol), sodium iodide (32.9 mg, 0.22 mmol) and K₂CO₃ (55.2 mg, 0.399 mmol) in MeCN (1.6 mL) and the mixture was heated at 80° C. overnight. The mixture was cooled down to rt, quenched with water, and extracted with EtOAc (×3). Reunited organic phases were dried over anhydrous sodium sulfate, filtered, evaporated to dryness and purified by silica gel column chromatography eluting with methanol (+1% NH₃ in MeOH) in dichloromethane from 1% to 10% to give Compound 153 (71 mg, yield 58%) as a white solid.

### The Following Compound was Synthesized by an Analogous Method as Described for Compound 153

| Compound No. | Structure | Starting Materials |
|---|---|---|
| Compound 154 | 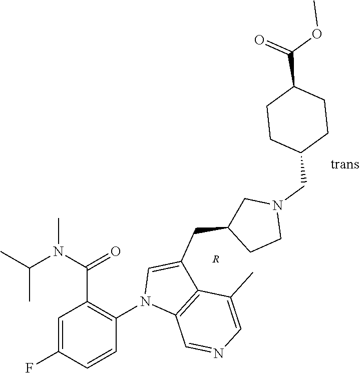 | Intermediate 202 & 223 |

### Preparation of Compound 162:

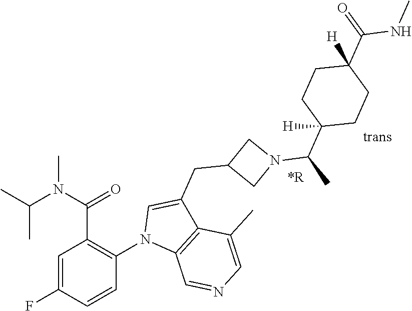 [SMILES: CNC(=O)[C@H]1CC[C@H]([C@@H](C)N2CC(Cc3cn(-c4ccc(F)cc4C(=O)N(C)C(C)C)c4cncc(C)c34)C2)CC1]

A stir bar, Compound 526a (50.0 mg, 0.089 mmol) and methanamine (2 mL, 30% in ethanol) were added to a 8 mL glass bottle. The reaction mixture was heated and stirred at 70° C. for 4 days. The reaction mixture was cooled down to room temperature and concentrated under reduced pressure to give a residue, which was purified by preparative-HPLC (Column: Boston Prime C18 150*30 mm*5 um, Mobile Phase A: water (CH₃COOH+CH₃COONH₄), Mobile Phase B: acetonitrile, Flow rate: 25 mL/min, gradient condition from 35% B to 65% B). The pure fractions were collected and the solvent was evaporated under vacuum. The residue was partitioned between acetonitrile (2 mL) and water (10 mL). The mixture was lyophilized to dryness to give Compound 162 (20.0 mg, 97.66% purity, 39.13% yield) as a white powder.

### The Following Compound was Synthesized by an Analogous Method as Described for Compound 162

| Compound No. | Structure | Starting Materials |
|---|---|---|
| Compound 163 | 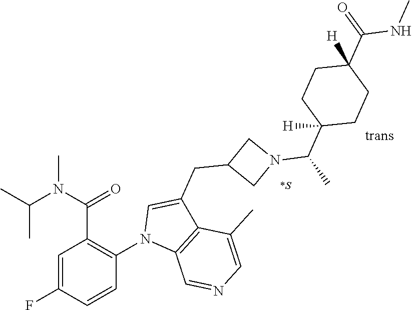 [SMILES: CNC(=O)[C@H]1CC[C@H]([C@H](C)N2CC(Cc3cn(-c4ccc(F)cc4C(=O)N(C)C(C)C)c4cncc(C)c34)C2)CC1] | Compound 526b |

### Preparation of Compound 172:

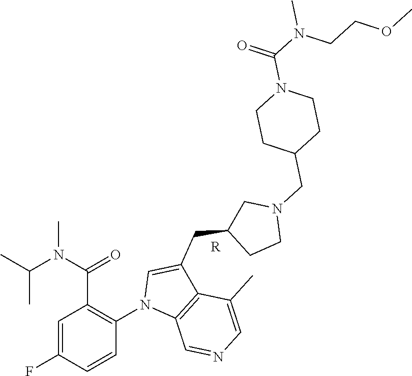

To a solution of Compound 448 (30 mg, 0.0593 mmol) and DIPEA (0.102 mL, 0.59 mmol) in DCM (6 mL) was added triphosgene (48.1 mg, 0.162 mmol). The mixture was stirred at r.t. for 0.5 hour. 2-methoxy-N-methylethan-1-amine (5.288 mg, 0.0593 mmol) was added and the mixture was stirred for further 2 hours. The solvent was removed and the residue was dissolved in MeOH (3 ml) and purified by preparative-HPLC (Column: Waters Sunfire C18 5 μm, 19*150 mm, Mobile Phase A: water (0.1% HCOOH), Mobile Phase B: acetonitrile, Flow rate: 17 mL/min, gradient condition from 0% B to 30% B) to afford Compound 172 (10 mg, 23% yield).

### Preparation of Compound 184, 184a & 184b:

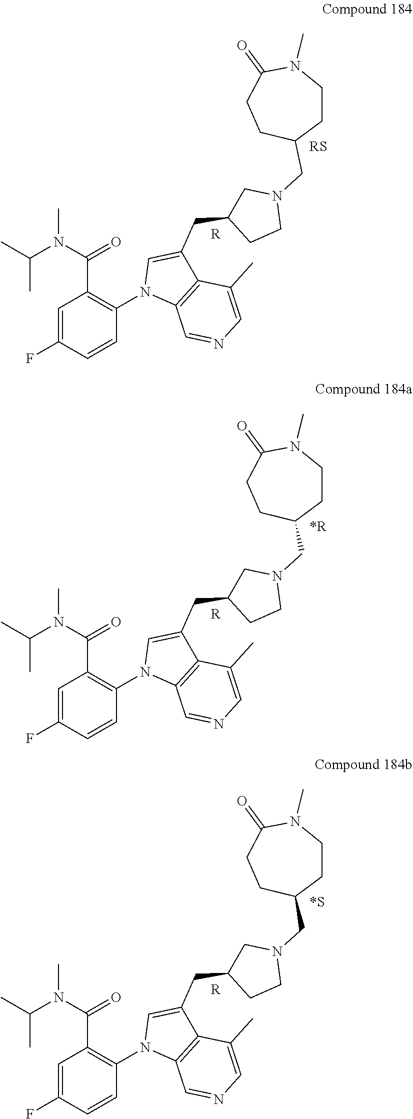

NaH (60% dispersion in mineral oil) (7.9 mg, 0.197 mmol) was added, under nitrogen at 0° C., to a solution of Compound 169 (70 mg, 0.131 mmol) in anhydrous DMF (1 mL). After 10 min, Mel (9.8 μL, 0.157 mmol) was added, and the reaction was left under stirring at rt overnight. The reaction was quenched with ice, diluted with MeOH to give the crude product, which was used as such for reversed-phase prep HPLC purification (Stationary phase: RP Xbridge Prep C18 OBD-5 μm, 50×250 mm, Mobile phase: 0.5% NH₄HCO₃ solution in water, CH₃CN) to give Compound 184 (30.5 mg, 41.1% yield) as a white solid. A purification was performed via Prep. SFC (Stationary phase: Chiralpak Diacel AD 20×250 mm, Mobile phase: CO₂, EtOH-iPrOH (50-50)+0.4% iPrNH₂). The first fraction was collected as Compound 184a (9.5 mg, 13% yield) and the second fraction as Compound 184b (9.5 mg, 13% yield) as white solids.

### Preparation of Compound 205, 205a & 205b:

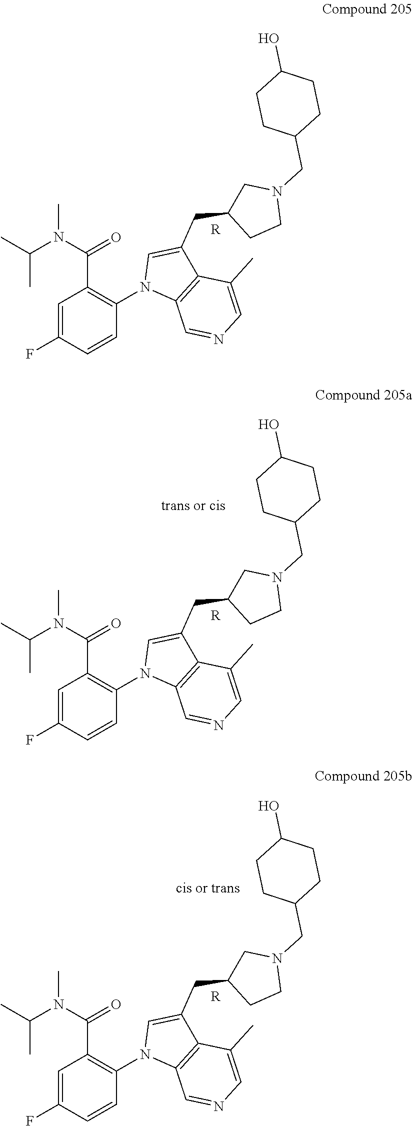

NaBH₄ (9.6 mg, 0.25 mmol) was added to a stirred solution of intermediate 264 (66 mg, 0.127 mmol) in MeOH (1.2 mL) at r.t. and the mixture was left under stirring for 20 min. The reaction was quenched with water and purified by Prep. HPLC to give Compound 205 (41 mg, yield 61.9%) as a white solid. A further purification was performed via Prep. SFC (Stationary phase: Chiralpak Diacel AD 20×250 mm, Mobile phase: CO₂, EtOH-iPrOH (50-50)+0.4% iPrNH₂) to give Compound 205a (25 mg, 38% yield) and Compound 205b (7 mg, 10.6% yield).

### Preparation of Compound 207:

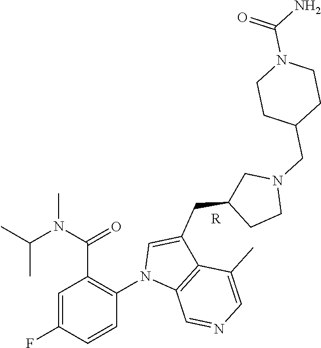

Compound 448 (120 mg, 0.237 mmol) and DIPEA (0.12 mL, 0.71 mmol) were added to DCM (5 mL). Isocyanatotrimethylsilane (32.8 mg, 0.28 mmol) was added and the mixture was stirred at r.t. for 16 hours. The solvent was removed and the residue was purified by preparative-HPLC (Column: Waters Xbridge C18 5 μm, 19*150 mm, Mobile Phase A: water (0.1% NH₄OH+10 mM NH₄HCO₃), Mobile Phase B: acetonitrile, Flow rate: 17 mL/min, gradient condition from 25% B to 35% B) to give Compound 207 (100 mg, 75% yield).

The Following Compounds were Synthesized by an Analogous Method as Described for Compound 207

| Compound No. | Structure | Starting Materials |
|---|---|---|
| Compound 217 | 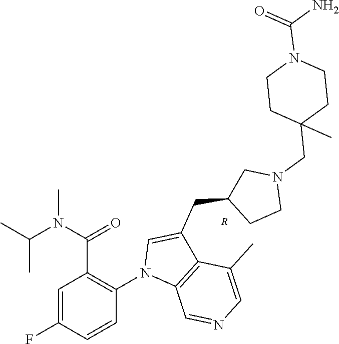 | Compound 468 |
| Compound 218 | 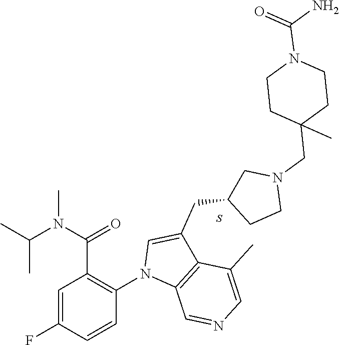 | Compound 469 |
| Compound 219 | 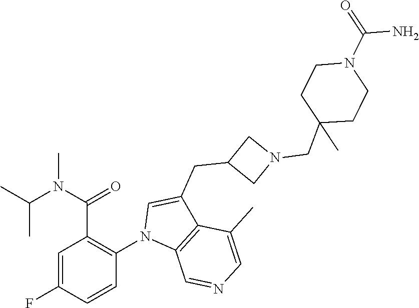 [SMILES: Cc1cncc2c1c(CC1CN(CC3(C)CCN(C(N)=O)CC3)C1)cn2-c1ccc(F)cc1C(=O)N(C)C(C)C] | Compound 430 |

### Preparation of Compound 261:

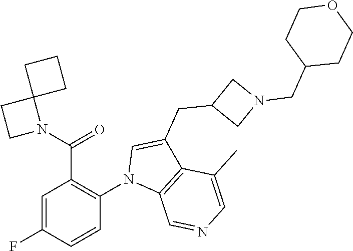 [SMILES: Cc1cncc2c1c(CC1CN(CC3CCOCC3)C1)cn2-c1ccc(F)cc1C(=O)N1CCC12CCC2]

1-azaspiro[3.3]heptane (0.165 mmol, 1.2 eq.) was pre-weighed into a 2-dram vial. A stock solution (23 mL) of intermediate 366 (1.38 g, 0.14 M), HATU (1.8 g, 0.2 M) and DIPEA (1.32 mL, 0.36 M) was prepared in DMF and stirred for 1 h. A 2nd stock solution of DIPEA (1.32 mL in 11.5 mL DMF) was also prepared. The DIPEA stock solution (0.5 mL) was added to each vial to solubilize the amine HCl salt. Intermediate 366/HATU/DIPEA solution (1 mL) was then added to each amine well. The reactions were stirred for 2 h, whereupon an extra 1.5 eq. HATU was added, and stirring continued overnight. The solvent was evaporated, and the samples redissolved in DMSO (0.5 mL) and MeCN (2.5 mL) for purification. Purification was performed via Prep HPLC (Stationary phase: RP XBridge Prep C18 OBD-10 μm, 30×150 mm, Mobile phase: 0.25% NH₄HCO₃ solution in water, CH₃CN) to give Compound 261 (3.6 mg, 4.8% yield) after lyophilization.

The Following Compounds were Synthesized by an Analogous Method as Described for Compound 261

Alternative purification methods that can be employed for the purification of examples listed below are as follows:

Purifications can also be performed via Prep HPLC (Stationary phase: RP Xselect CSH Prep C18 OBD-10 μm, 30×150 mm, Mobile phase: 0.1% FA solution in water, CH₃CN or MeOH).

Purifications can also be performed via Prep HPLC: (Stationary phase: RP Xbridge Prep C18 OBD-10 μm, 30×150 mm, Mobile phase: 0.25% NH₄HCO₃ solution in water, MeOH).

Purifications can also be performed via Prep SFC (Stationary phase: Torus Diol 30×150 mm, Mobile phase: CO₂, MeOH+20 mM NH₄OH).

These purification methods can also be used in combination.

| Compound No. | Structure | Starting Materials |
|---|---|---|
| Compound 262 | 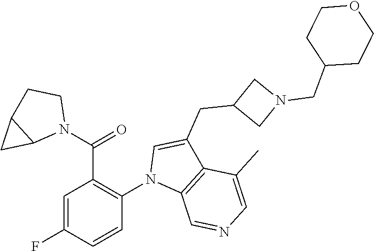 [SMILES: Cc1cncc2c1c(CC1CN(CC3CCOCC3)C1)cn2-c1ccc(F)cc1C(=O)N1CCC2CC21] | intermediate 366 & 2- azabicyclo[3.1.0]hexane hydrochloride |
| Compound 263 | 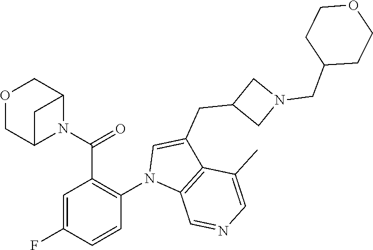 [SMILES: Cc1cncc2c1c(CC1CN(CC3CCOCC3)C1)cn2-c1ccc(F)cc1C(=O)N1C2COCC1C2] | intermediate 366 & 3-oxa-6-aza- bicyclo[3.1.1]heptane tosylate |
| Compound 264 | 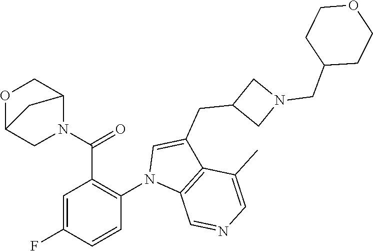 [SMILES: Cc1cncc2c1c(CC1CN(CC3CCOCC3)C1)cn2-c1ccc(F)cc1C(=O)N1CC2CC1CO2] | intermediate 366 & 2-oxa-5- azabicyclo[2.2.1]heptane, hydrochloride (1:1) |
| Compound 265 | 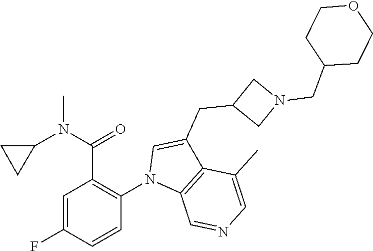 [SMILES: Cc1cncc2c1c(CC1CN(CC3CCOCC3)C1)cn2-c1ccc(F)cc1C(=O)N(C)C1CC1] | intermediate 366 & N- cyclopropylmethylamine |
| Compound 266 | 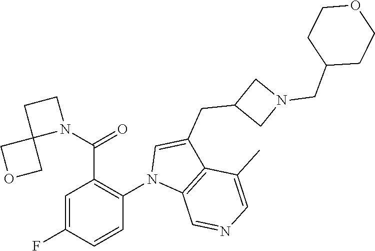 [SMILES: Cc1cncc2c1c(CC1CN(CC3CCOCC3)C1)cn2-c1ccc(F)cc1C(=O)N1CCC12COC2] | intermediate 366 & 6-oxa-1- azaspiro[3.3]heptane oxalate(2:1) |
| Compound 267 | 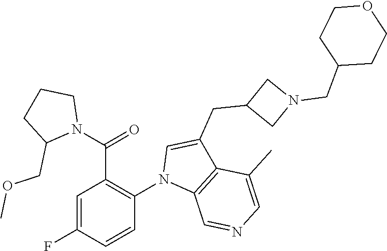 [SMILES: COCC1CCCN1C(=O)c1cc(F)ccc1-n1cc(CC2CN(CC3CCOCC3)C2)c2c(C)cncc21] | intermediate 366 & 2- (methoxymethyl)pyrrolidine |
| Compound 268 | 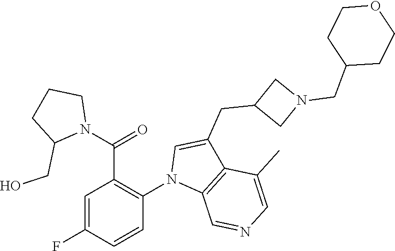 [SMILES: Cc1cncc2c1c(CC1CN(CC3CCOCC3)C1)cn2-c1ccc(F)cc1C(=O)N1CCCC1CO] | intermediate 366 & 2- (hydroxymethyl)pyrrolidine |
| Compound 269 | 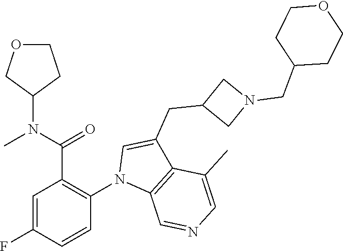 [SMILES: Cc1cncc2c1c(CC1CN(CC3CCOCC3)C1)cn2-c1ccc(F)cc1C(=O)N(C)C1CCOC1] | intermediate 366 & N- methyltetrahydrofuran-3-amine |
| Compound 270 | 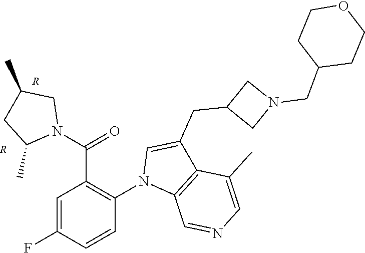 | intermediate 366 & (3R,5R)-5- methylpyrrolidin-3-ol hydrochloride |
| Compound 271 | 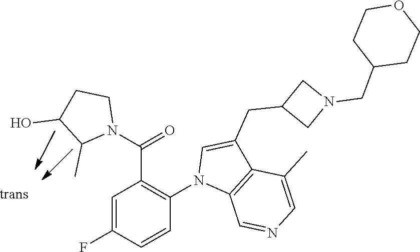 [SMILES: Cc1cncc2c1c(CC1CN(CC3CCOCC3)C1)cn2-c1ccc(F)cc1C(=O)N1CCC(O)C1C] | intermediate 366 & rel-(2R,3S)- 2-methylpyrrolidin-3-ol hydrochloride |
| Compound 272 | 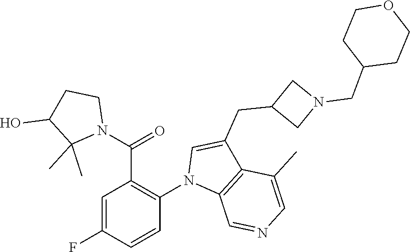 [SMILES: Cc1cncc2c1c(CC1CN(CC3CCOCC3)C1)cn2-c1ccc(F)cc1C(=O)N1CCC(O)C1(C)C] | intermediate 366 & 2,2- dimethylpyrrolidin-3-ol |
| Compound 273 | 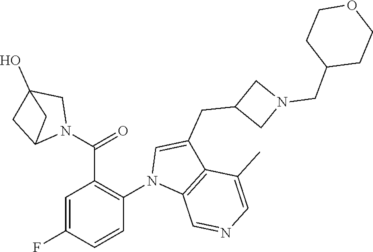 [SMILES: Cc1cncc2c1c(CC1CN(CC3CCOCC3)C1)cn2-c1ccc(F)cc1C(=O)N1CC2(O)CC1C2] | intermediate 366 & 2- azabicyclo[2.1.1]hexan-4-ol hydrochloride |
| Compound 274 | 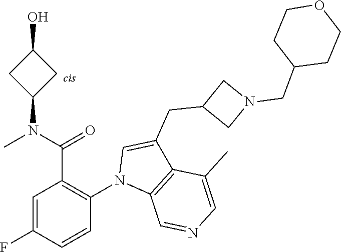 [SMILES: Cc1cncc2c1c(CC1CN(CC3CCOCC3)C1)cn2-c1ccc(F)cc1C(=O)N(C)[C@H]1C[C@@H](O)C1] | intermediate 366 & cis-3- (methylamino)cyclobutan-1-ol hydrochloride |
| Compound 275 | 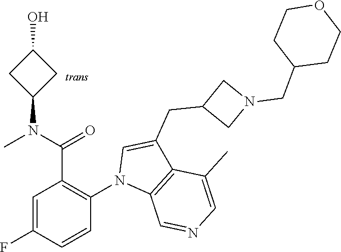 [SMILES: Cc1cncc2c1c(CC1CN(CC3CCOCC3)C1)cn2-c1ccc(F)cc1C(=O)N(C)[C@H]1C[C@H](O)C1] | intermediate 366 & trans-3- (methylamino)cyclobutanol |
| Compound 276 | 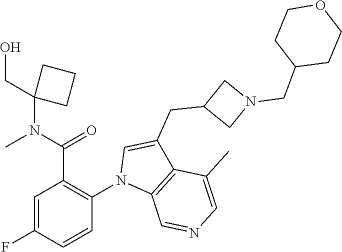 [SMILES: Cc1cncc2c1c(CC1CN(CC3CCOCC3)C1)cn2-c1ccc(F)cc1C(=O)N(C)C1(CO)CCC1] | intermediate 366 & [1- (methylamino)cyclobutyl] methanol |
| Compound 277 | 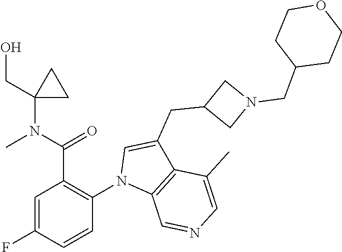 [SMILES: Cc1cncc2c1c(CC1CN(CC3CCOCC3)C1)cn2-c1ccc(F)cc1C(=O)N(C)C1(CO)CC1] | intermediate 366 & (1- (methylamino)cyclopropyl) methanol hydrochloride |
| Compound 278 | 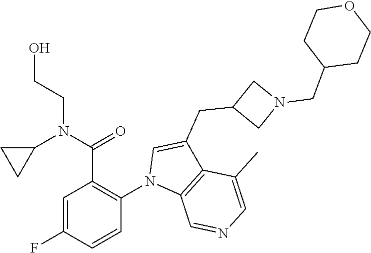 [SMILES: Cc1cncc2c1c(CC1CN(CC3CCOCC3)C1)cn2-c1ccc(F)cc1C(=O)N(CCO)C1CC1] | intermediate 366 & 2- (cyclopropylamino)ethanol |
| Compound 279 | 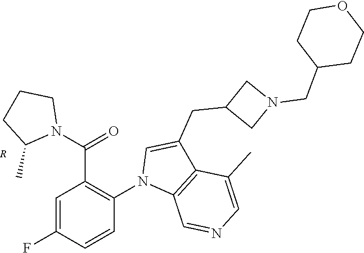 | intermediate 366 & (R)-(−)-2- methylpyrrolidine |
| Compound 280 | 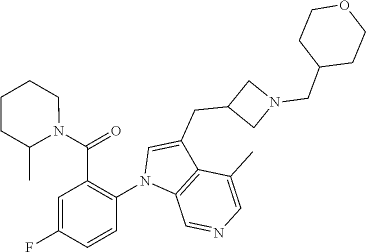 [SMILES: Cc1cncc2c1c(CC1CN(CC3CCOCC3)C1)cn2-c1ccc(F)cc1C(=O)N1CCCCC1C] | intermediate 366 & 2- methylpiperidine |
| Compound 281 | 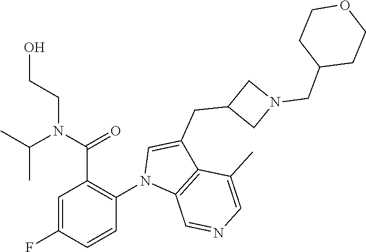 [SMILES: Cc1cncc2c1c(CC1CN(CC3CCOCC3)C1)cn2-c1ccc(F)cc1C(=O)N(CCO)C(C)C] | intermediate 366 & 2- (isopropylamino)ethanol |
| Compound 282 | 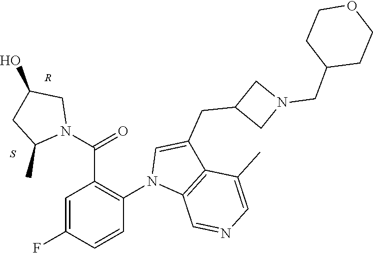 | intermediate 366 & (3R,5S)-5- methylpyrrolidin-3-ol hydrochloride |

### Preparation of Compound 283:

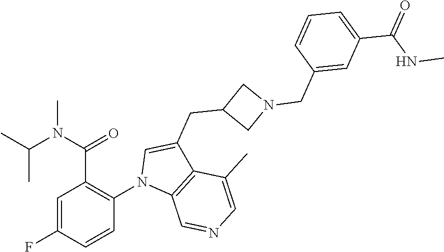 [SMILES: CNC(=O)c1cccc(CN2CC(Cc3cn(-c4ccc(F)cc4C(=O)N(C)C(C)C)c4cncc(C)c34)C2)c1]

The 3-formyl-N-methylbenzamide (0.4 mmol, 2 eq.) was pre-weighed into a 2-dram vial with a stirrer bar. Stock solutions of intermediate 25 (0.79 g, 7.5 mL, 0.27 M) and sodium cyanoborohydride (0.23 g, 7.5 mL, 0.48 M) were prepared in MeOH. 0.75 mL of intermediate stock solution was added and the solutions stirred for 2 h. Next, sodium cyanoborohydride stock solution (0.75 mL) was then added. The reaction mixture was then stirred at room temperature overnight. After reaction completion, the solution was added to MeOH-washed ethylbenzenesulfonic acid resin cartridge (Isolute® SCX-3), and eluted with MeOH (3×2 mL) followed by 3.5 M NH₃ in MeOH (3×2 mL). The basic washes containing the product was evaporated and re-dissolved in 3 mL 1:1 MeCN/MeOH for purification. Purification was performed via Prep SFC (Stationary phase: Torus Diol 30×150 mm, Mobile phase: CO₂, MeOH+20 mM NH₄OH) to give Compound 283 (41 mg, 38% yield), after lyophilization.

The Following Compounds were Synthesized by an Analogous Method as Described for Compound 283

For reactions employing ketone building blocks, the following applies: acetic acid was added (23 μL, 2 eq.) into the reaction mixture before the addition of the sodium cyanoborohydride stock solution. The reaction mixture was stirred at 50° C. overnight (during the reductive amination step).

Alternative purification methods that can be employed for the purification of examples listed below are as follows:

Purifications can also be performed via Prep HPLC (Stationary phase: RP Xselect CSH Prep C18 OBD-10 μm, 30×150 mm, Mobile phase: 0.1% FA solution in water, CH₃CN or MeOH).

Purifications can also be performed via Prep HPLC: (Stationary phase: RP Xbridge Prep C18 OBD-10 μm, 30×150 mm, Mobile phase: 0.25% NH₄HCO₃ solution in water, CH₃CN or MeOH).

These purification methods can also be used in combination.

| Compound No. | Structure | Starting Materials |
|---|---|---|
| Compound 284 | 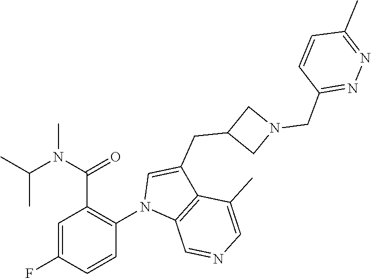 [SMILES: Cc1ccc(CN2CC(Cc3cn(-c4ccc(F)cc4C(=O)N(C)C(C)C)c4cncc(C)c34)C2)nn1] | Intermediate 25 & 6- methylpyridazine-3- carbaldehyde |
| Compound 285 | 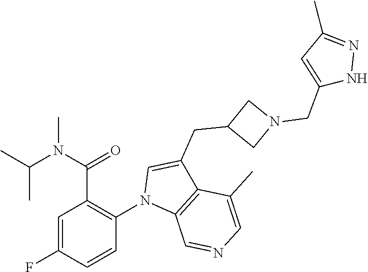 [SMILES: Cc1cc(CN2CC(Cc3cn(-c4ccc(F)cc4C(=O)N(C)C(C)C)c4cncc(C)c34)C2)[nH]n1] | Intermediate 25 & 5-methyl-1H- pyrazole-3-carbaldehyde |
| Compound 286 | 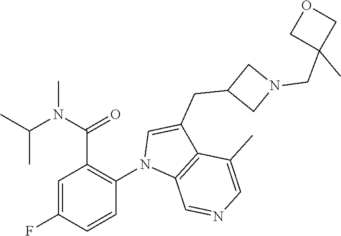 [SMILES: Cc1cncc2c1c(CC1CN(CC3(C)COC3)C1)cn2-c1ccc(F)cc1C(=O)N(C)C(C)C] | Intermediate 25 & 3- methyloxetane-3-carbaldehyde |
| Compound 287 | 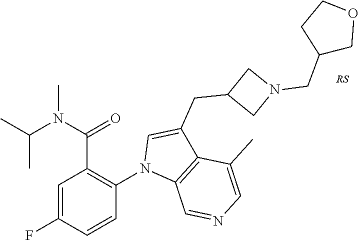 [SMILES: Cc1cncc2c1c(CC1CN(CC3CCOC3)C1)cn2-c1ccc(F)cc1C(=O)N(C)C(C)C] | Intermediate 25 & tetrahydrofuran-3- carboxaldehyde |
| Compound 288 | 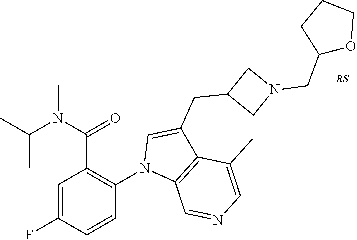 [SMILES: Cc1cncc2c1c(CC1CN(CC3CCCO3)C1)cn2-c1ccc(F)cc1C(=O)N(C)C(C)C] | Intermediate 25 & tetrahydro-2- furancarbaldehyde |
| Compound 289 | 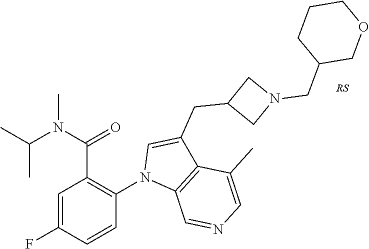 [SMILES: Cc1cncc2c1c(CC1CN(CC3CCCOC3)C1)cn2-c1ccc(F)cc1C(=O)N(C)C(C)C] | Intermediate 25 & tetrahydropyran-3-carbaldehyde |
| Compound 290 | 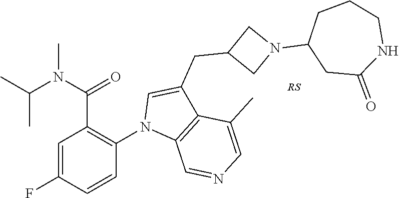 [SMILES: Cc1cncc2c1c(CC1CN(C3CCCNC(=O)C3)C1)cn2-c1ccc(F)cc1C(=O)N(C)C(C)C] | Intermediate 25 & azepane-2,4- dione |
| Compound 291 | 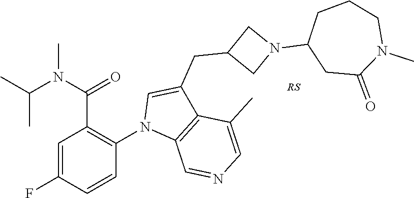 [SMILES: Cc1cncc2c1c(CC1CN(C3CCCN(C)C(=O)C3)C1)cn2-c1ccc(F)cc1C(=O)N(C)C(C)C] | Intermediate 25 & 1- methylazepane-2,4-dione |

### Preparation of Compound 292a:

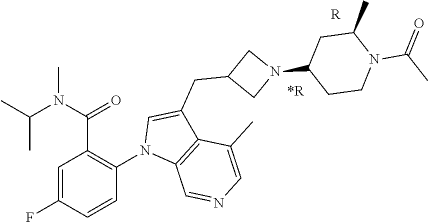

(R)-tert-Butyl 2-methyl-4-oxopiperidine-1-carboxylate (0.4 mmol, 2 eq.) was pre-weighed into a 2-dram vial with a stirrer bar. Stock solutions of intermediate 25 (0.63 g, 6.0 mL, 0.27 M) and sodium cyanoborohydride (0.18 g, 6.0 mL, 0.48 M) were prepared in MeOH. 0.75 mL of intermediate 25 stock solution was added to the reaction vial, together with acetic acid (23 μL, 2 eq.) and the solutions stirred for 1 h. The sodium cyanoborohydride stock solution (0.75 mL) was then added. The reaction mixtures were stirred at 50° C. overnight. After reaction completion, the solutions were added to MeOH-washed ethylbenzenesulfonic acid resin cartridge (Isolute® SCX-3), and eluted with MeOH (3×2 mL) followed by 3.5 M NH₃ in MeOH (3×2 mL). The basic washes containing the product were evaporated.

The crude products from the reductive amination were dissolved in DCM (1 mL) and TFA (2 mL), and stirred at 50° C. for 1 h. The solvents were evaporated and redissolved in MeCN (2 mL). Siliamet® Diamine resin was added and the mixture stirred for 0.5 h. The resin was removed via filtration on a 24-well filter plate, and the filtrate concentrated.

The Boc deprotected products were dissolved in 1 mL DCM, and DIPEA (0.55 mL, 3.2 mmol), and Ac₂O (0.25 mL, 2.6 mmol) were added. The reaction mixture was stirred for 2 h at room temperature, at which time they were quenched with MeOH (2 mL) and concentrated. The samples were re-dissolved in 3 mL 1:1 MeCN/MeOH for purification. Purification was performed via Prep SFC (Stationary phase: Torus Diol 30×150 mm, Mobile phase: CO₂, MeOH+20 mM NH₄OH) to give Compound 292a (22.9 mg, yield: 21%), after lyophilization.

The Following Compounds were Synthesized by an Analogous Method as Described for Compound 292a

For reactions employing ketone building blocks, the following applies: acetic acid was added (23 μL, 2 eq.) into the reaction mixture before the addition of the sodium cyanoborohydride stock solution. The reaction mixture was stirred at 50° C. overnight (during the reductive amination step).

Alternative purification methods that can be employed for the purification of examples listed below are as follows:

Purifications can also be performed via Prep HPLC (Stationary phase: RP Xselect CSH Prep C18 OBD-10 μm, 30×150 mm, Mobile phase: 0.100 FA solution in water, CH₃CN or MeOH).

Purifications can also be performed via Prep HPLC: (Stationary phase: RP Xbridge Prep C18 OBD-10 μm, 30×150 mm, Mobile phase: 0.25% NH₄HCO₃ solution in water, CH₃CN or MeOH).

These purification methods can also be used in combination.

| Compound No. | Structure | Starting Materials |
|---|---|---|
| Compound 292b | 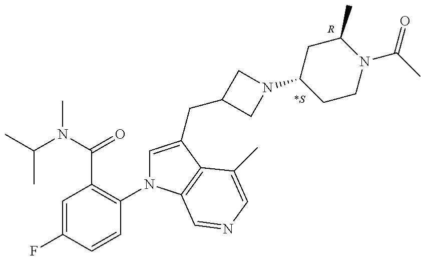 | Intermediate 25 & (R)-tert-Butyl 2-methyl-4-oxopiperidine-1- carboxylate |
| Compound 293a | 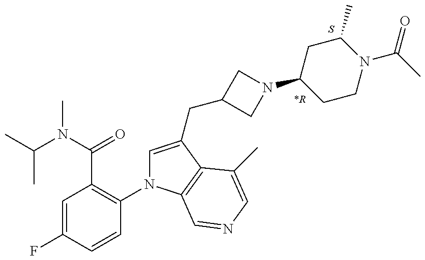 | Intermediate 25 & (S)-tert-Butyl 2-methyl-4-oxopiperidine-1- carboxylate |
| Compound 293b | 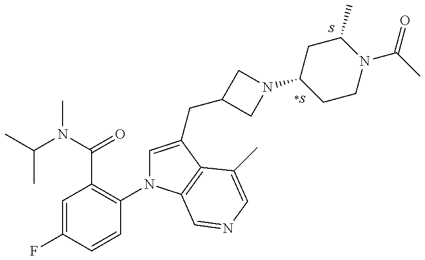 | Intermediate 25 & (S)-tert-Butyl 2-methyl-4-oxopiperidine-1- carboxylate |
| Compound 294a | 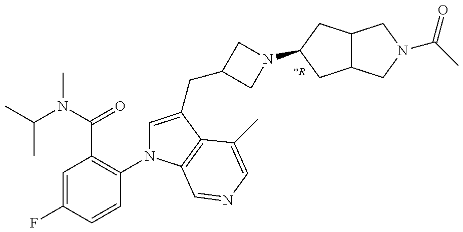 | Intermediate 25 & 2-Boc-5- oxohexahydrocyclopenta[c]pyrrole |
| Compound 294b | 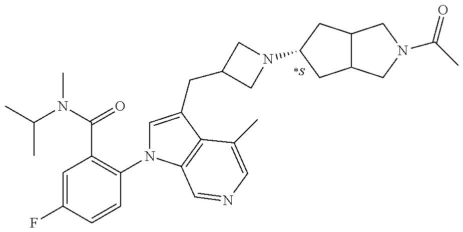 | Intermediate 25 & 2-Boc-5- oxohexahydrocyclopenta[c]pyrrole |
| Compound 295 | 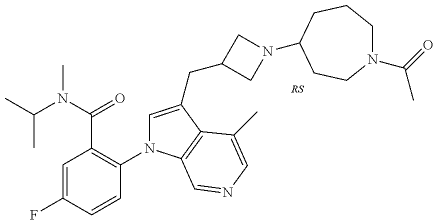 [SMILES: CC(=O)N1CCCC(N2CC(Cc3cn(-c4ccc(F)cc4C(=O)N(C)C(C)C)c4cncc(C)c34)C2)CC1] | Intermediate 25 & N-Boc- hexahydro-1H-azepin-4-one |
| Compound 296 | 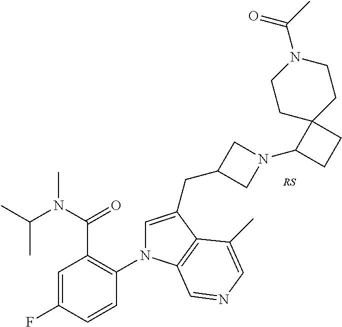 [SMILES: CC(=O)N1CCC2(CCC2N2CC(Cc3cn(-c4ccc(F)cc4C(=O)N(C)C(C)C)c4cncc(C)c34)C2)CC1] | Intermediate 25 & tert-butyl 1- oxo-7-azaspiro[3.5]nonane-7- carboxylate |

### Preparation of Intermediate 393:

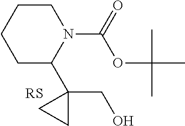 [SMILES: CC(C)(C)OC(=O)N1CCCCC1C1(CO)CC1]

1-(1-[(tert-butoxy)carbonyl]piperidin-2-yl)cyclopropane-1-carboxylic acid (0.40 g, 1.5 mmol) was dissolved in anhydrous THF (20 mL). Then, the mixture was cooled to 0° C. in an ice bath. Next, BH3-THF (1M solution, 2.2 mL, 2.2 mmol) was added dropwise. The mixture was then allowed to stir at ambient temperature for ˜2 h, after which LC/MS showed full conversion of the starting material. Then, water was carefully added. After gas formation ceased, solid K2CO3 (0.26 g) was added and the mixture stirred at ambient temperature for −30 min. Then, EA was added and the mixture transferred to a separatory funnel. The layers were separated and the water layer was extracted twice more with EA. Organic layers were combined, dried over Na2SO4, filtered and evaporated. The residue was purified by silica gel chromatography eluting with ethyl acetate in petroleum ether from 30% to 80% to give Intermediate 393 (0.36 g, 1.4 mmol, yield: 95%) as an oil.

The Following Intermediates were Synthesized by an Analogous Method as Described for Intermediate 393

| Intermediate No. | Structure | Starting Materials |
|---|---|---|
| Intermediate 394 | 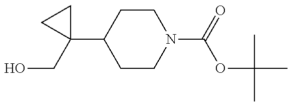 [SMILES: CC(C)(C)OC(=O)N1CCC(C2(CO)CC2)CC1] | 1-Boc-(1carboxy-cyclopropyl)- piperidine |
| Intermediate 395 | 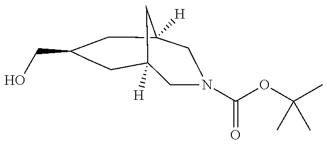 [SMILES: CC(C)(C)OC(=O)N1C[C@H]2C[C@H](CO)CC[C@@H](C2)C1] | 3-(1,1-Dimethylethyl)(7-exo)-3- azabicyclo[3.3.1]nonane-3,7- dicarboxylate |

### Preparation of Intermediate 396:

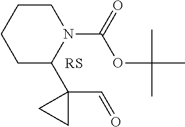 [SMILES: CC(C)(C)OC(=O)N1CCCCC1C1(C=O)CC1]

Intermediate 393 (0.15 g, 0.59 mmol) was dissolved in ethyl acetate (4 mL). Next, IBX (0.49 g, 1.8 mmol) was added and the resulting mixture was heated to 80° C., open to air. After 3 h, TLC analysis (50% EA/Heptane) showed full conversion of the starting material. The mixture was cooled to ambient temperature, and filtered. The filter cake was washed once with EA. The filtrate was evaporated to dryness to give intermediate 396 (0.13 g, 0.51 mmol, yield: 87%).

The Following Intermediates were Synthesized by an Analogous Method as Described for Intermediate 396

| Intermediate No. | Structure | Starting Materials |
|---|---|---|
| Intermediate 397 | 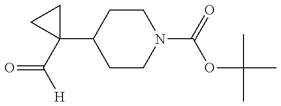 [SMILES: CC(C)(C)OC(=O)N1CCC(C2(C=O)CC2)CC1] | Intermediate 394 |
| Intermediate 398 | 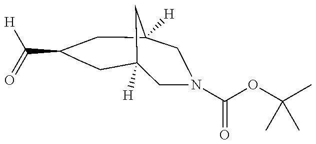 [SMILES: CC(C)(C)OC(=O)N1C[C@H]2CC[C@@H](C=O)C[C@@H](C2)C1] | Intermediate 395 |

### Preparation of Compound 297:

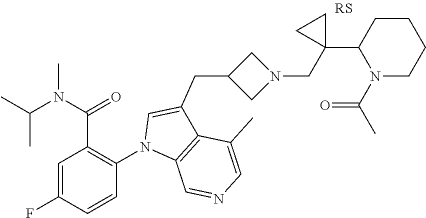 [SMILES: CC(=O)N1CCCCC1C1(CN2CC(Cc3cn(-c4ccc(F)cc4C(=O)N(C)C(C)C)c4cncc(C)c34)C2)CC1]

Intermediate 396 (0.4 mmol, 2 eq.) was pre-weighed into 2-dram vials with a stirrer bar. Stock solutions of intermediate 25 (0.63 g, 6.0 mL, 0.27 M) and sodium cyanoborohydride (0.18 g, 6.0 mL, 0.48 M) were prepared in MeOH. 0.75 mL of intermediate 25 stock solution was added to the vial and the solution stirred for 1 h. The sodium cyanoborohydride stock solution (0.75 mL) was then added. The reaction mixture was stirred at room temperature (for aldehydes). After reaction completion, the solutions were added to MeOH-washed ethylbenzenesulfonic acid resin cartridge (Isolute® SCX-3) cartridge, and eluted with MeOH (3×2 mL) followed by 3.5 M NH₃ in MeOH (3×2 mL). The basic washes containing the product were evaporated to dryness.

The crude products from the reductive amination were dissolved in DCM (1 mL) and TFA (2 mL), and stirred at 50° C. for 1 h. The solvents were evaporated and redissolved in MeCN (2 mL). Siliamet® Diamine resin was added and the mixtures stirred for 0.5 h. The resin was removed via filtration on a 24-well filter plate, and the filtrate concentrated.

The Boc deprotected products were dissolved in 1 mL DCM, and DIPEA (0.55 mL, 3.2 mmol), and Ac₂O (0.25 mL, 2.6 mmol) were added. The reaction mixtures were stirred for 2 h at room temperature, at which time they were quenched with MeOH (2 mL) and concentrated. The samples were re-dissolved in 3 mL 1:1 MeCN/MeOH for purification. Purification was performed via Prep SFC (Stationary phase: Torus Diol 30×150 mm, Mobile phase: CO₂, MeOH+20 mM NH₄OH) to give Compound 297 (91 mg, yield=79%) after lyophilization.

The Following Compounds were Synthesized by an Analogous Method as Described for Compound 297 For reactions employing ketone building blocks, the following applies: acetic acid was added (23 μL, 2 eq.) into the reaction mixture before the addition of the sodium cyanoborohydride stock solution. The reaction mixture was stirred at 50° C. overnight (during the reductive amination step).

Alternative purification methods that can be employed for the purification of examples listed below are as follows:

Purifications can also be performed via Prep HPLC (Stationary phase: RP Xselect CSH Prep C18 OBD-10 μm, 30×150 mm, Mobile phase: 0.1% FA solution in water, CH₃CN or MeOH).

Purifications can also be performed via Prep HPLC: (Stationary phase: RP Xbridge Prep C18 OBD-10 μm, 30×150 mm, Mobile phase: 0.25% NH₄HCO₃ solution in water, CH₃CN or MeOH).

These purification methods can also be used in combination.

| Compound No. | Structure | Starting Materials |
|---|---|---|
| Compound 298 | 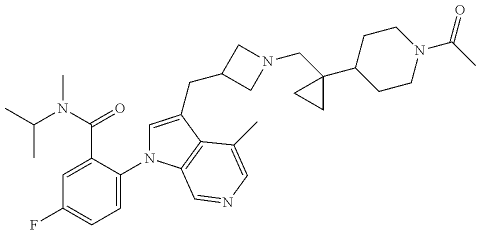 [SMILES: CC(=O)N1CCC(C2(CN3CC(Cc4cn(-c5ccc(F)cc5C(=O)N(C)C(C)C)c5cncc(C)c45)C3)CC2)CC1] | Intermediate 25 & Intermediate 397 |
| Compound 299a | 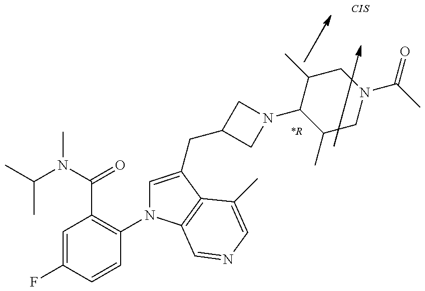 [SMILES: "CIS" ; CC(=O)N1CC(C)C(N2CC(Cc3cn(-c4ccc(F)cc4C(=O)N(C)C(C)C)c4cncc(C)c34)C2)C(C)C1] | Intermediate 25 & tert-butyl cis 3,5-dimethyl-4-oxopiperidine-1- carboxylate |
| Compound 300 | 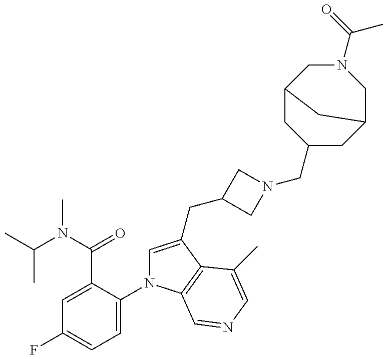 [SMILES: CC(=O)N1CC2CC(CN3CC(Cc4cn(-c5ccc(F)cc5C(=O)N(C)C(C)C)c5cncc(C)c45)C3)CC(C2)C1] | Intermediate 25 & Intermediate 398 |
| Compound 301 | 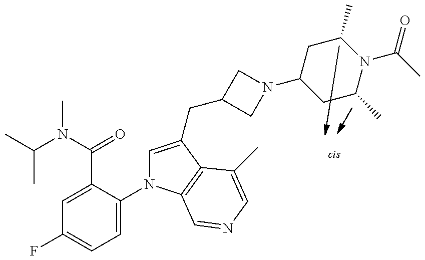 [SMILES: CC(=O)N1[C@H](C)CC(N2CC(Cc3cn(-c4ccc(F)cc4C(=O)N(C)C(C)C)c4cncc(C)c34)C2)C[C@@H]1C] | Intermediate 25 & cis-2,6- dimethyl-4-oxo-piperidine-1- carboxylic acid tert-butyl ester |
| Compound 302 | 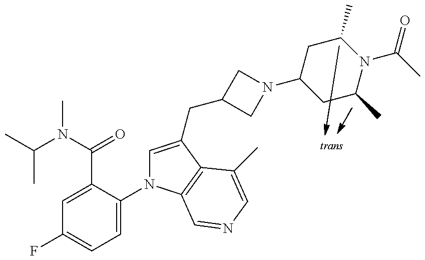 [SMILES: CC(=O)N1[C@@H](C)CC(N2CC(Cc3cn(-c4ccc(F)cc4C(=O)N(C)C(C)C)c4cncc(C)c34)C2)C[C@@H]1C] | Intermediate 25 & trans-2,6- dimethyl-4-oxo-piperidine-1- carboxylic acid tert-butyl ester |
| Compound 303 | 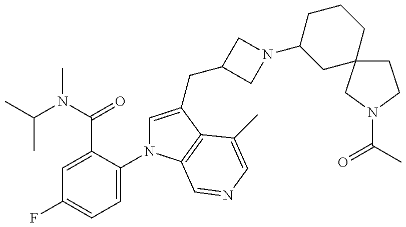 [SMILES: CC(=O)N1CCC2(CCCC(N3CC(Cc4cn(-c5ccc(F)cc5C(=O)N(C)C(C)C)c5cncc(C)c45)C3)C2)C1] | Intermediate 25 & 2-Boc-7-oxo- 2-azaspiro[4.5]decane |
| Compound 304 | 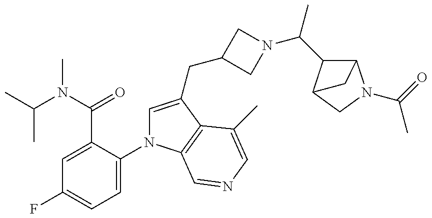 [SMILES: CC(=O)N1CC2CC1C2C(C)N1CC(Cc2cn(-c3ccc(F)cc3C(=O)N(C)C(C)C)c3cncc(C)c23)C1] | Intermediate 25 & (1R,4S,5S)- rel-tert-Butyl 5-acetyl-2- azabicyclo[2.1.1]hexane-2- carboxylate |
| Compound 305 | 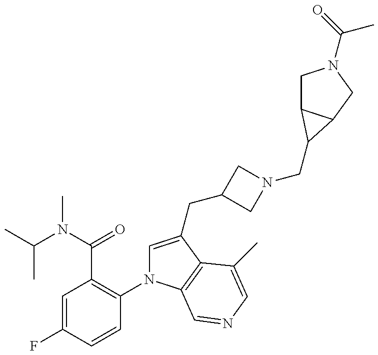 [SMILES: CC(=O)N1CC2C(CN3CC(Cc4cn(-c5ccc(F)cc5C(=O)N(C)C(C)C)c5cncc(C)c45)C3)C2C1] | Intermediate 25 & tert-butyl rel- (1R,5S,6s)-6-formyl-3- azabicyclo[3.1.0]hexane-3- carboxylate |
| Compound 306 | 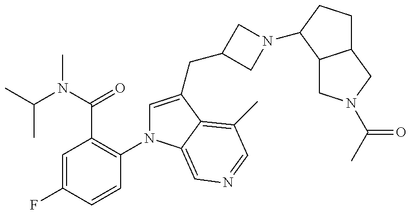 [SMILES: CC(=O)N1CC2CCC(N3CC(Cc4cn(-c5ccc(F)cc5C(=O)N(C)C(C)C)c5cncc(C)c45)C3)C2C1] | Intermediate 25 & 4- oxohexahydrocyclopenta[c]pyrrole- 2-carboxylic acid tert-butyl ester |
| Compound 307 | 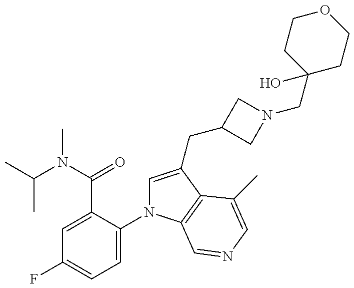 [SMILES: Cc1cncc2c1c(CC1CN(CC3(O)CCOCC3)C1)cn2-c1ccc(F)cc1C(=O)N(C)C(C)C] | Intermediate 25 & 4- hydroxytetrahydro-2H-pyran-4- carbaldehyde |
| Compound 308 | 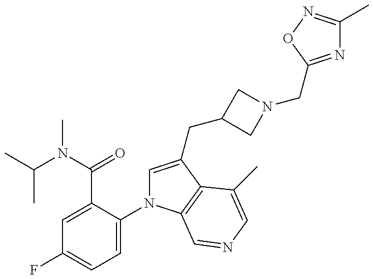 [SMILES: Cc1noc(CN2CC(Cc3cn(-c4ccc(F)cc4C(=O)N(C)C(C)C)c4cncc(C)c34)C2)n1] | Intermediate 25 & 3-methyl- 1,2,4-oxadiazole-5-carbaldehyde |
| Compound 309a | 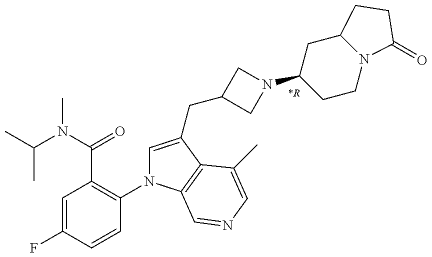 [SMILES: Cc1cncc2c1c(CC1CN([C@@H]3CCN4C(=O)CCC4C3)C1)cn2-c1ccc(F)cc1C(=O)N(C)C(C)C] | Intermediate 25 & hexahydroindolizine-3.7-dione |
| Compound 309b | 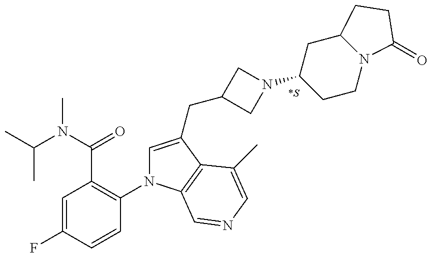 [SMILES: Cc1cncc2c1c(CC1CN([C@H]3CCN4C(=O)CCC4C3)C1)cn2-c1ccc(F)cc1C(=O)N(C)C(C)C] | Intermediate 25 & hexahydroindolizine-3,7-dione |
| Compound 299b | 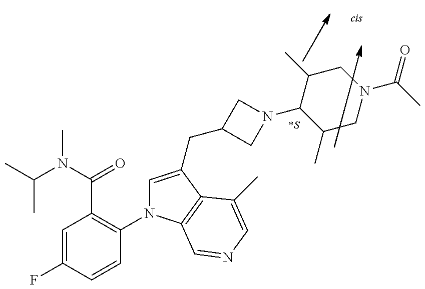 [SMILES: CC(=O)N1CC(C)C(N2CC(Cc3cn(-c4ccc(F)cc4C(=O)N(C)C(C)C)c4cncc(C)c34)C2)C(C)C1] | Intermediate 25 & tert-butyl cis 3,5-dimethyl-4-oxopiperidine-1- carboxylate |

### Preparation of Compound 310:

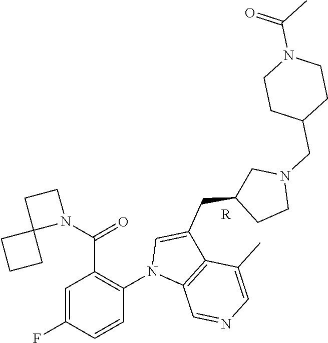

1-azaspiro[3.3]heptane hydrochloride (0.17 mmol, 1.2 eq.) were pre-weighed into a 2-dram vial. A stock solution (21 mL) of intermediate 370 (1.26 g, 0.12 M), HATU (1.46 g, 0.18 M) and DIPEA (1.1 mL) was prepared in DMF and stirred for 1 h. A 2nd stock solution of DIPEA (1.1 mL in 10.5 mL DMF) was also prepared. The DIPEA stock solution (0.5 mL) was added to each the vial to solubilize the amine HCl salt. Intermediate 370/HATU/DIPEA solution (1 mL) was then added. The reaction was stirred overnight at room temperature. The DMF was removed by evaporation. The crude was redissolved in DCM/EtOAc 2/1 (2.2 mL), and water (2.2 mL) was added. The mixture was stirred for 10 minutes. Then, the mixture was left standing for 10 minutes. 2 mL of the organics were removed using a pipette and these were filtered over a fritted filter. The remaining water layer was extracted another time using 2 mL of the DCM/EtOAc mixture. Again, 2 mL was removed and filtered over the same fritted filter. The fritted filter was rinsed with 500 μL of DMSO. The obtained filtrates were concentrated in vacuo until only DMSO remained. The obtained crude were redissolved in MeOH/MeCN 1/1 (2.2 mL) and submitted for purification. Purification was performed via Prep HPLC (Stationary phase: RP XBridge Prep C18 OBD-10 μm, 30×150 mm, Mobile phase: 0.25% NH₄HCO₃ solution in water, CH₃CN) to give Compound 310 (8.7 mg, yield: 12%) after lyophilization.

The Following Compounds were Synthesized by an Analogous Method as Described for Compound 310

Alternative purification methods that can be employed for the purification of examples listed below are as follows:

Purifications can also be performed via Prep HPLC (Stationary phase: RP Xselect CSH Prep C18 OBD-10 μm, 30×150 mm, Mobile phase: 0.1% FA solution in water, CH₃CN or MeOH).

Purifications can also be performed via Prep HPLC: (Stationary phase: RP Xbridge Prep C18 OBD-10 μm, 30×150 mm, Mobile phase: 0.25% NH₄HCO₃ solution in water, MeOH).

Purifications can also be performed via Prep SFC (Stationary phase: Torus Diol 30×150 mm, Mobile phase: CO₂, MeOH+20 mM NH₄OH).

These purification methods can also be used in combination.

| Compound No. | Structure | Starting Materials |
|---|---|---|
| Compound 311 | 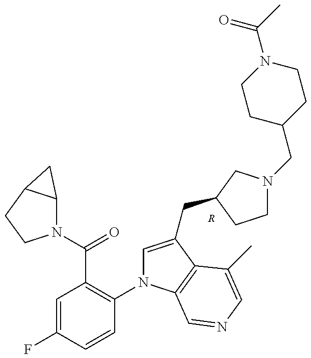 | Intermediate 370 & 2- azabicyclo[3.1.0]hexane hydrochloride |
| Compound 311a & Compound 311b | 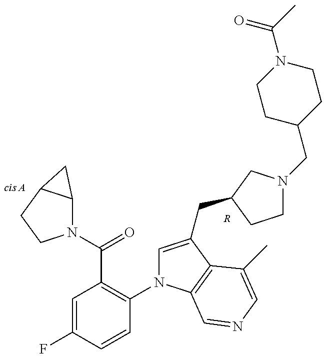 | Compound 311 was separated by Prep SFC (Stationary phase: Chiralpak Diacel AD 20 × 250 mm, Mobile phase: CO₂, EtOH- iPrOH (50-50) + 0.4% iPrNH₂). The first fraction was collected as Compound 311a & the second fraction as Compound 311b. |
| | 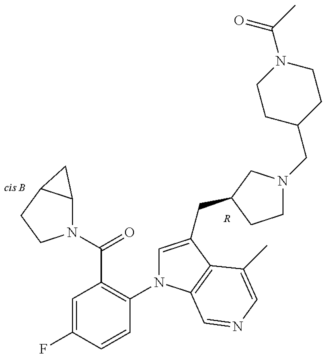 | |
| Compound 312 | 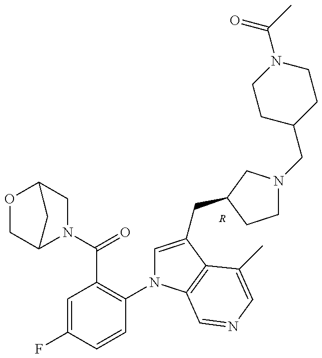 | Intermediate 370 & 2-oxa-5- azabicyclo[2.2.1]heptane HCl |
| Compound 313 | 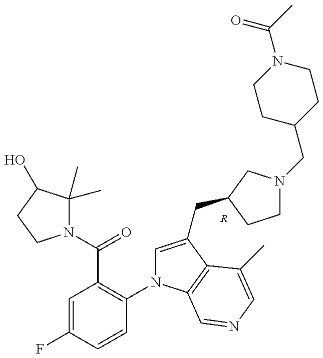 | Intermediate 370 & 2,2- dimethylpyrrolidin-3-ol |
| Compound 313a & Compound 313b | 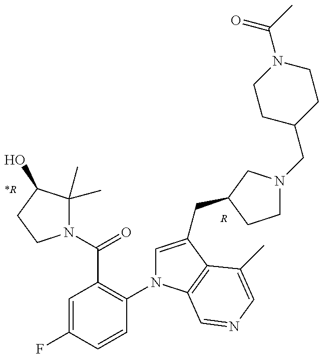 | Compound 313 was separated by Prep SFC (Stationary phase: Chiralpak Daicel ID 20 × 250 mm, Mobile phase: CO₂, iPrOH + 0.4 iPrNH₂). The first fraction was collected as Compound 313a & the second fraction as Compound 313b. |
| Compound 314 | 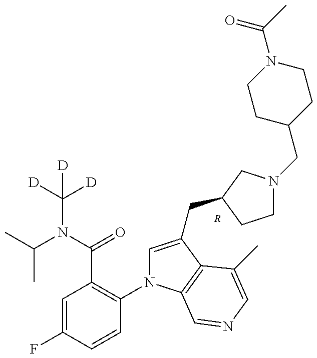 | Intermediate 370 & N-(methyl- d₃)propan-2-amine |
| Compound 315 | 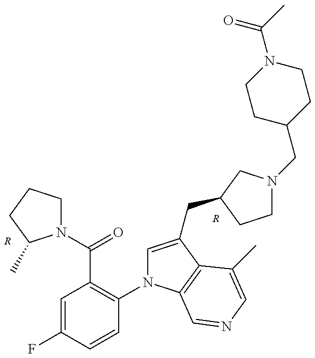 | Intermediate 370 & (R)-(−)-2- methylpyrrolidine |
| Compound 316 | 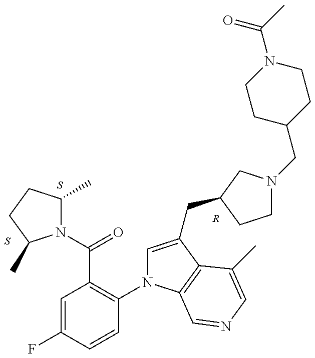 | Intermediate 370 & (2S,5S)-2,5- dimethylpyrrolidine hydrochloride |
| Compound 317 | 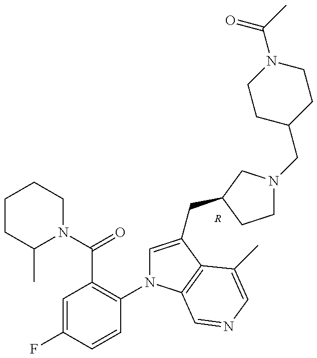 | Intermediate 370 & 2- methylpiperidine |
| Compound 318 | 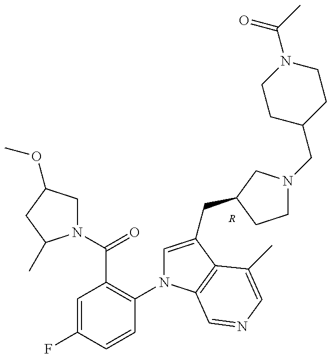 | Intermediate 370 & 4-methoxy- 2-methyl-pyrrolidine |
| Compound 319 | 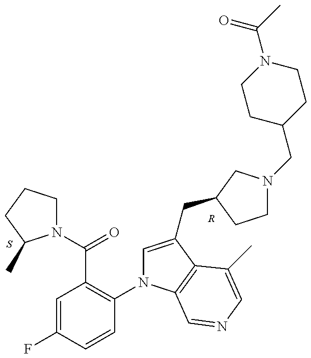 | Intermediate 370 & (S)-(+)-2- methylpyrrolidine |
| Compound 320 | 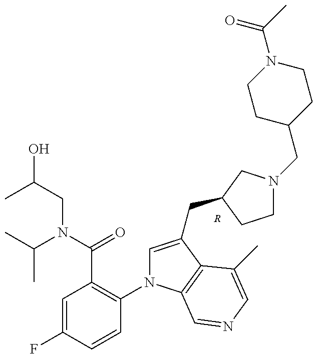 | Intermediate 370 & 1-(propan-2- ylamino)propan-2-ol |
| Compound 320a & Compound 320b | 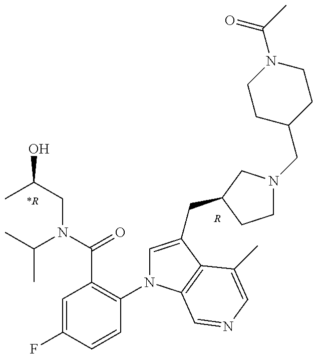 | Compound 320 was separated by Prep SFC(Stationary phase: Chiralpak Diacel AD 20 × 250 mm, Mobile phase: CO₂, iPrOH + 0.4 iPrNH₂). The first fraction was collected as Compound 320a & the second fraction as Compound 320b. |
| | 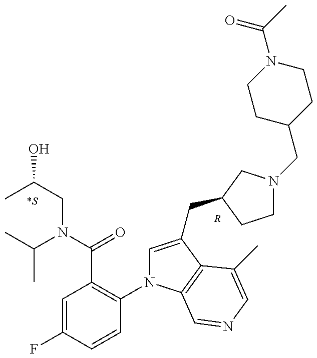 | |
| Compound 321 | 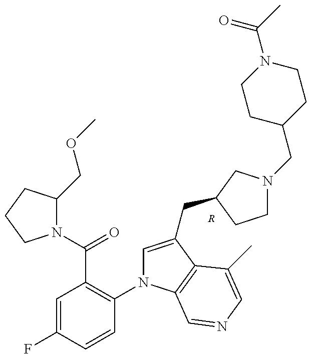 | Intermediate 370 & 2- (methoxymethyl)pyrrolidine |
| Compound 322 | 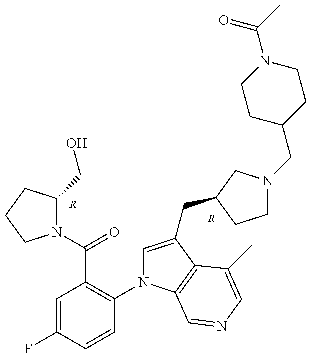 | Intermediate 370 & (R)-2- pyrrolidinemethanol |
| Compound 323 | 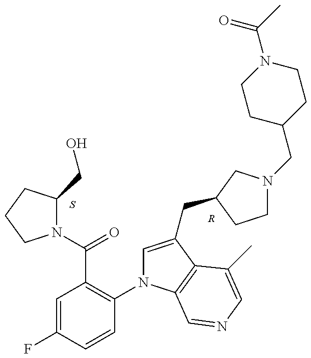 | Intermediate 370 & (S)-2- pyrrolidinemethanol |
| Compound 324 | 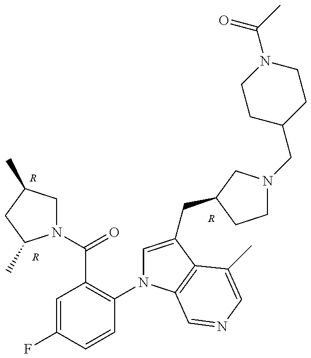 | Intermediate 370 & (3R,5R)-5- methylpyrrolidin-3-ol hydrochloride |
| Compound 325 | 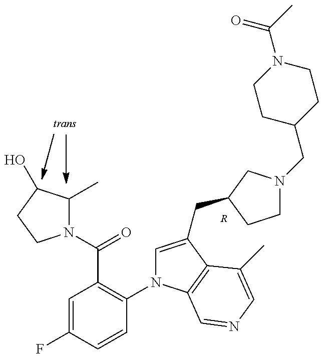 | Intermediate 370 & rel-(2R,3S)- 2-methylpyrrolidin-3-ol hydrochloride |
| Compound 326 | 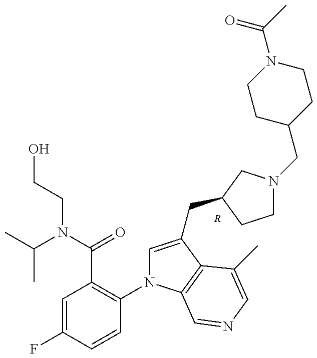 | Intermediate 370 & 2- (isopropylamino)ethanol |
| Compound 327 | 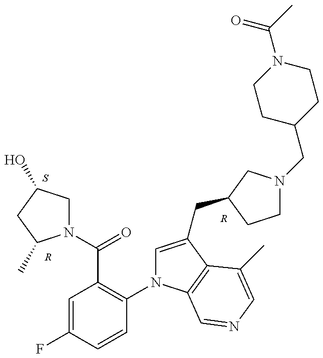 | Intermediate 370 & (2R,4S)-4- hydroxy-2-methylpyrrolidine hydrochloride |
| Compound 354 | 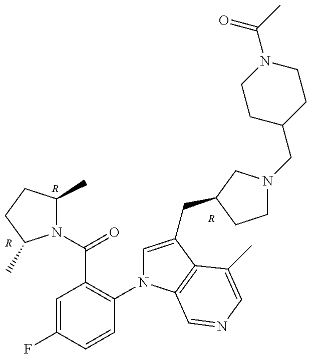 | Intermediate 370 & (2R,5R)-2,5-dimethylpyrrolidine |
| Compound 355 | 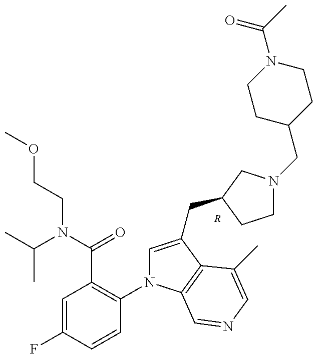 | Intermediate 370 & N-(2- methoxyethyl)propan-2-amine |
| Compound 356 | 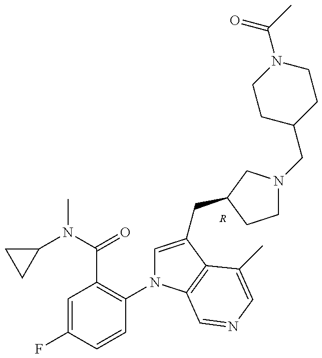 | Intermediate 370 & N- methylcyclopropanamine |

### Preparation of Compound 328:

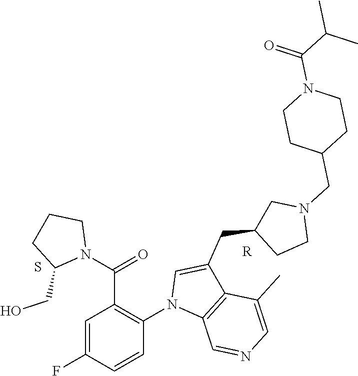

A stock solution of intermediate 374 (39.1 mg, 0.075 mmol), DIPEA (0.038 mL, 0.2 mmol) and HATU (42.8 mg, 0.1 mmol) in DMF (0.56 mL) was added to a pre-weighed (S)-(+)-2-pyrrolidinemethanol (0.15 mmol, 2 equiv). The resulting solution was stirred for 2 h at rt. Afterwards the solvent was removed under reduced pressure. To the vial was added 2 mL of a DCM/EtOAc=2/1 mix and 1 mL aq. 1N citric acid solution and mixture was stirred for 5 minutes. The organic phase was collected, and the solvent removed under reduced pressure. 3 mL of a 2/1 mix MeOH/MeCN was added. Purification was performed via Prep HPLC (Stationary phase: RP XBridge Prep C18 OBD-10 μm, 30×150 mm, Mobile phase: 0.25% NH₄HCO₃ solution in water, CH₃CN) to give Compound 328 (31 mg, 69% yield) after lyophilization.

The Following Compounds were Synthesized by an Analogous Method as Described for Compound 328

Alternative purification methods that can be employed for the purification of examples listed below are as follows:

Purifications can also be performed via Prep HPLC (Stationary phase: RP Xselect CSH Prep C18 OBD-10 μm, 30×150 mm, Mobile phase: 0.1% FA solution in water, CH₃CN or MeOH).

Purifications can also be performed via Prep HPLC: (Stationary phase: RP Xbridge Prep C18 OBD-10 μm, 30×150 mm, Mobile phase: 0.25% NH₄HCO₃ solution in water, MeOH).

Purifications can also be performed via Prep SFC (Stationary phase: Torus Diol 30×150 mm, Mobile phase: CO₂, MeOH+20 mM NH₄OH).

These purification methods can also be used in combination.

| Compound No. | Structure | Starting Materials |
|---|---|---|
| Compound 329 | 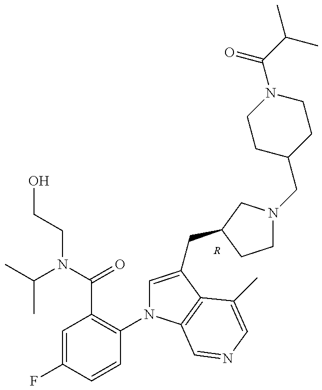 | Intermediate 374 & 2- (isopropylamino)ethanol |
| Compound 330 | 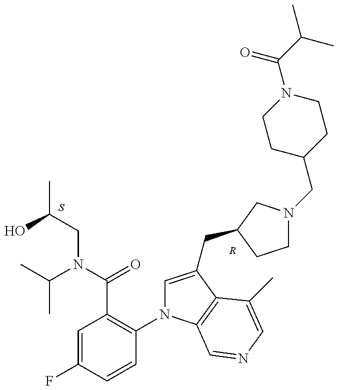 | Intermediate 374 & (2S)-1-[(1- methylethyl)amino]-2-propanol |
| Compound 331 | 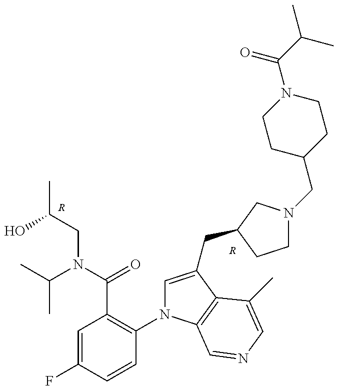 | Intermediate 374 & (2R)-1-[(1- methylethyl)amino]-2-propanol |
| Compound 332 | 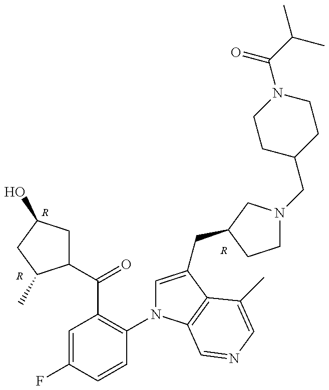 | Intermediate 374 & (3R,5R)-5- methylpyrrolidin-3-ol hydrochloride |
| Compound 333 | 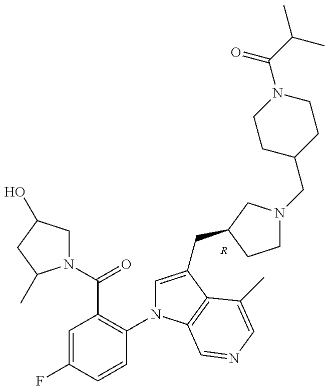 | Intermediate 374 & 5- methylpyrrolidin-3-ol |
| Compound 334 | 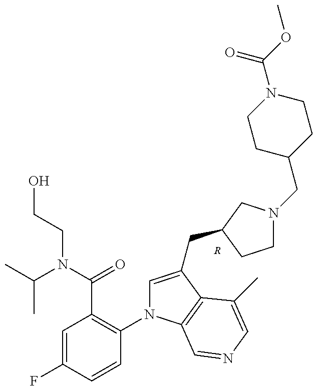 | Intermediate 376 & 2- (isopropylamino)ethanol |
| Compound 335 | 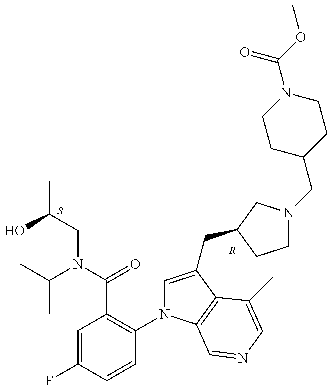 | Intermediate 376 & (2S)-1-[(1- methylethyl)amino]-2-propanol |
| Compound 336 | 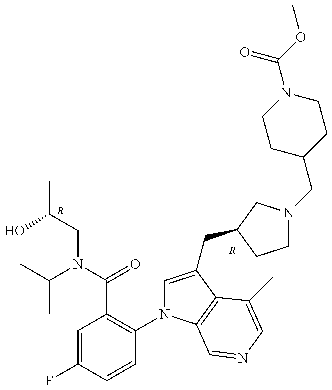 | Intermediate 376 & (2R)-1-[(1- methylethyl)amino]-2-propanol |
| Compound 337 | 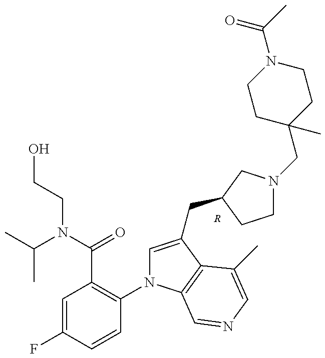 | Intermediate 380 & 2- (isopropylamino)ethanol |
| Compound 338 | 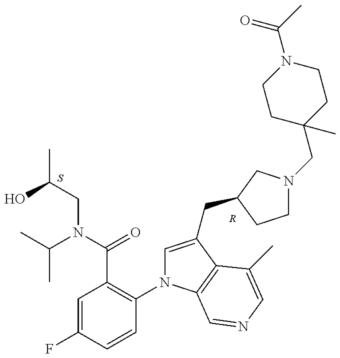 | Intermediate 380 & (2S)-1-[(1- methylethyl)amino]-2-propanol |
| Compoumd 339 | 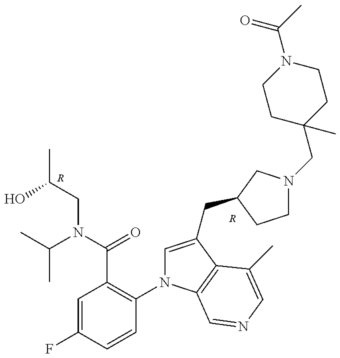 | Intermediate 380 & (2R)-1-[(1- methylethyl)amino]-2-propanol |
| Compound 340 | 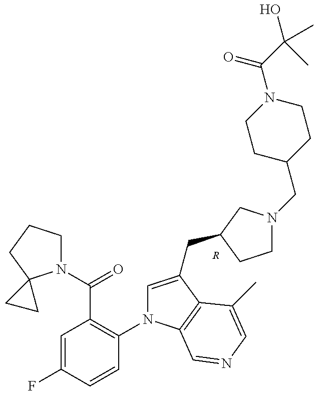 | Intermediate 382 & 4- azaspiro[2.4]heptane hydrochloride |
| Compound 341 | 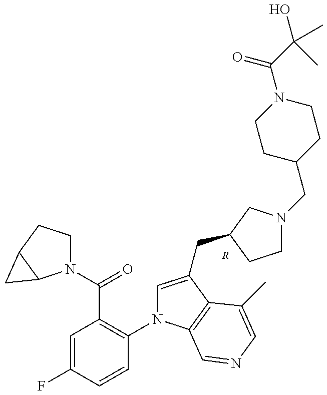 | Intermediate 382 & 2- azabicyclo[3.1.0]hexane hydrochloride |
| Compound 342 | 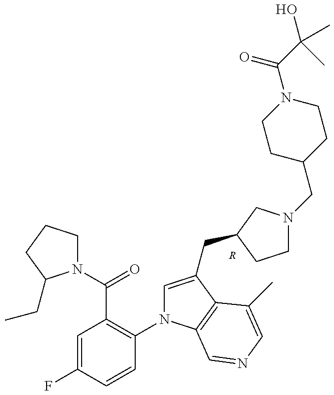 | Intermediate 382 & 2- ethylpyrrolidine |
| Compound 343 | 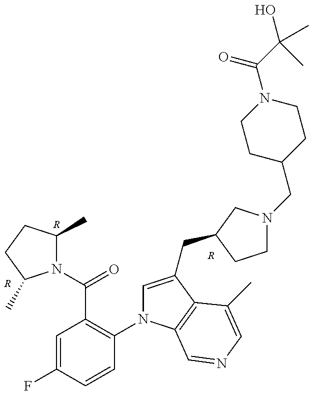 | Intermediate 382 & (2S,5S)-2,5- dimethylpyrrolidine hydrochloride |
| Compound 344 | 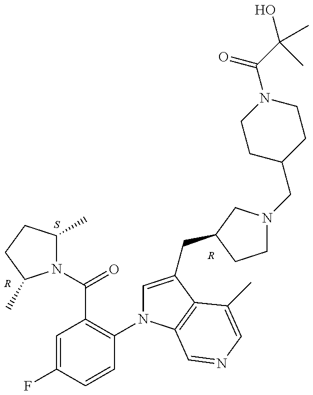 | Intermediate 382 & cis-2,5- dimethyl-pyrrolidine hydrochloride |
| Compound 345 | 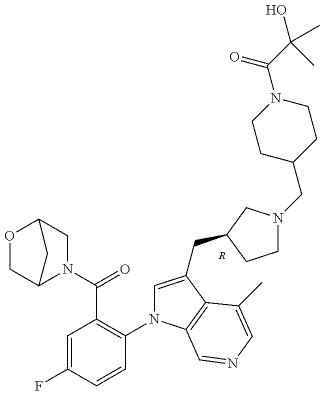 | Intermediate 382 & 2-oxa-5- azabicyclo[2.2.1]heptane |
| Compound 346 | 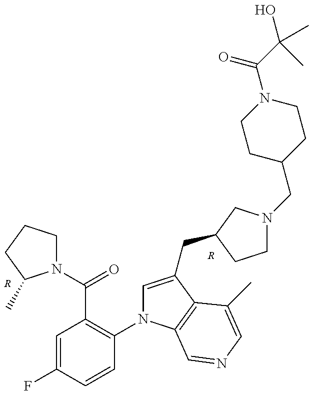 | Intermediate 382 & (R)-2- methylpyrrolidine-hydrochloride |
| Compound 347 | 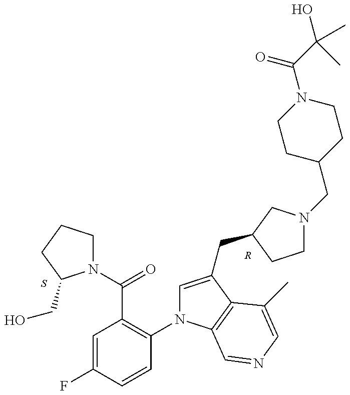 | Intermediate 384 & (S)-(+)-2- pyrrolidinemethanol |
| Compound 348 | 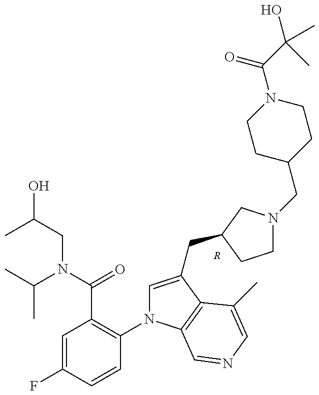 | Intermediate 384 & 1-(propan-2- ylamino)propan-2-ol |
| Compound 349 | 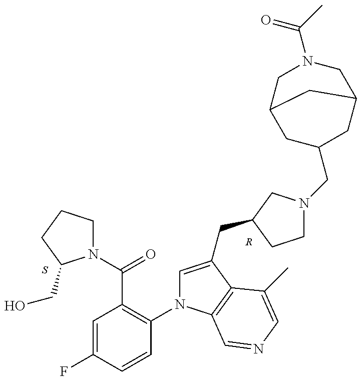 | Intermediate 388 & (S)-(+)-2- pyrrolidinemethanol |
| Compound 350 | 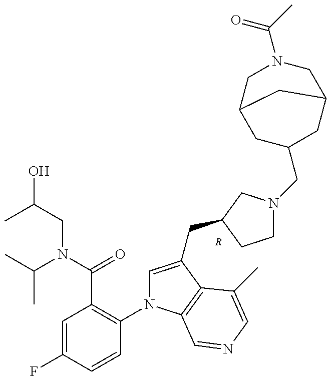 | Intermediate 388 & 1-(propan-2- ylamino)propan-2-ol |
| Compound 351a & Compound 351b | 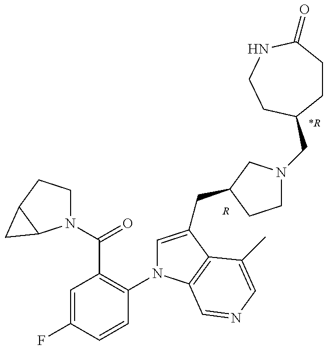 | Intermediate 309 & 2- azabicyclo[3.1.0]hexane hydrochloride The product was separated by Prep SFC (Stationary phase: Chiralcel Diacel IH 20 × 250 mm, Mobile phase: CO₂, EtOH + 0.4 iPrNH₂). The first fraction was collected as Compound 351a & the second fraction as Compound 351b. |
| | 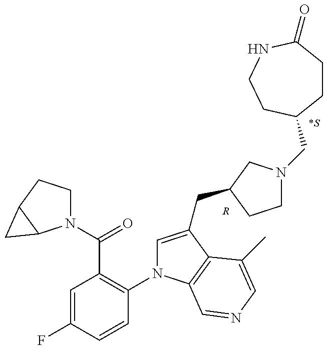 | |
| Compound 352a & Compound 352b | 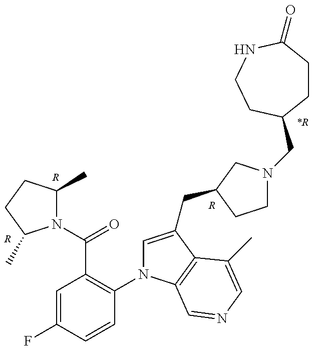 | Intermediate 390 & (2R,5R)-2,5- dimethylpyrrolidine hydrochloride The product was separated by Prep SFC (Stationary phase: Chiralcel Diacel IH 20 × 250 mm, Mobile phase: CO₂, EtOH + 0.4 iPrNH₂). The first fraction was collected as Compound 352a & the second fraction as Compound 352b. |
| | 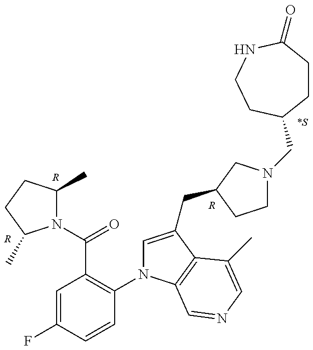 | |
| Compound 353a & Compound 353b | 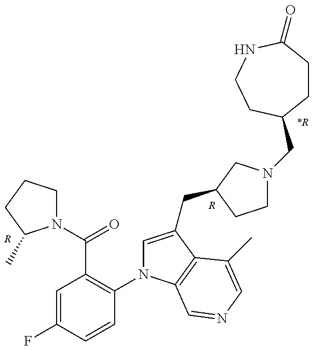 | Intermediate 390 & R-(−)-2- methylpyrrolidine The product was separated by Prep SFC (Stationary phase: Chiralcel Diacel IH 20 × 250 mm, Mobile phase: CO₂, EtOH + 0.4 iPrNH₂). The first fraction was collected as Compound 353a & the second fraction as Compound 353b. |
| | 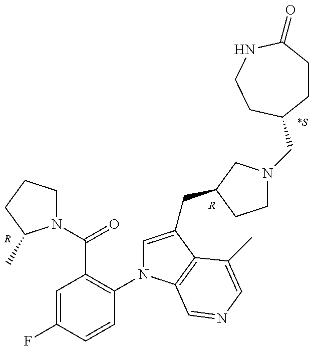 | |
| Compound 357 | 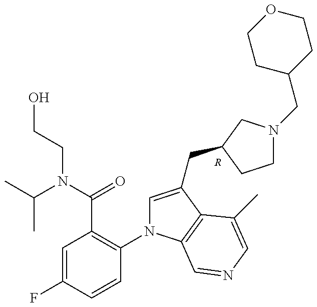 | Intermediate 392 & 2- (isopropylamino)ethan-1-ol |
| Compound 358 | 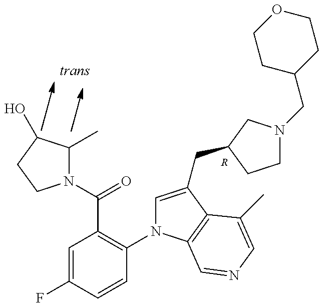 | Intermediate 392 & rel-(2R,3S)- 2-methylpyrrolidin-3-ol hydrochloride |
| Compound 359 | 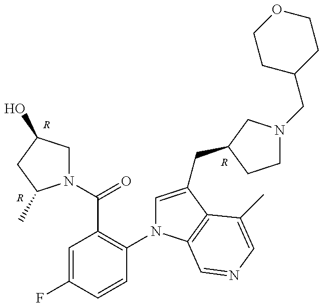 | Intermediate 392 & (3R,5R)-5- methylpyrrolidin-3-ol |
| Compound 360 | 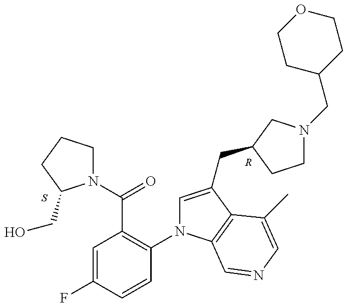 | Intermediate 392 & (S)- pyrrolidin-2-ylmethanol |
| Compound 361 | 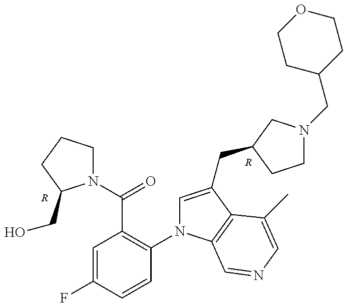 | Intermediate 392 & (R)- pyrrolidine-2-ylmethanol |
| Compound 362a & Compound 362b | 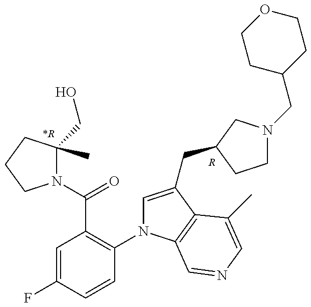 | Intermediate 392 & (2-methyl-2- pyrrolidinyl)methanol The product was separated by Prep SFC (Stationary phase: Torus Diol 30 × 150 mm, Mobile phase: CO₂, MeOH + 20 mM NH₄OH). The first fraction was collected as Compound 362a & the second fraction as Compound 362b. |
| | 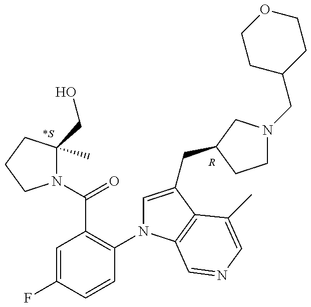 | |
| Compound 363a & Compound 363b | 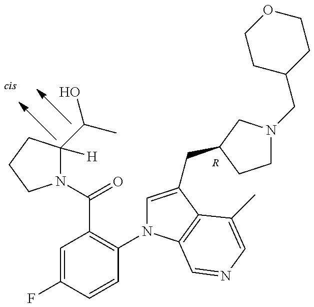 | Intermediate 392 & 1-[(2R)- pyrrolidin-2-yl]ethan-1-ol The product was separated by Prep SFC (Stationary phase: Chiralpak Daicel IG 20 × 250 mm, Mobile phase: CO₂, EtOH + 0.4 iPrNH₂). The first fraction was collected as Compound 363a & the second fraction as Compound 363b. |
| | 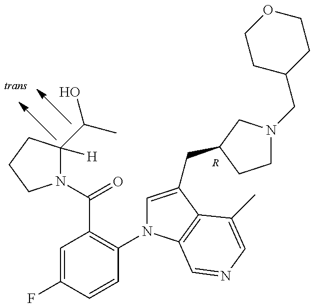 | |
| Compound 364a & Compound 364b | 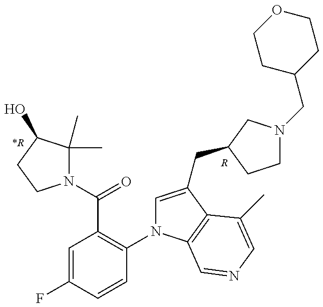 | Intermediate 392 & 2,2- dimethylpyrrolidin-3-ol The product was separated by Prep SFC (Stationary phase: Chiralcel Diacel OD 20 × 250 mm, Mobile phase: CO₂, MeOH + 0.4 iPrNH₂). The first fraction was collected as Compound 364a & the second fraction as Compound 364b. |
| | 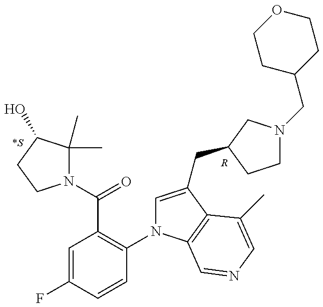 | |
| Compound 365a & Compound 365b | 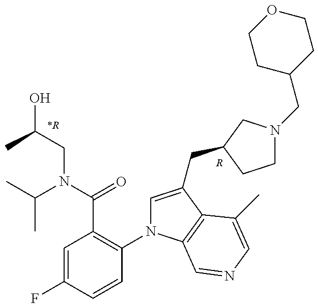 | Intermediate 392 & 1- isopropylamino-propan-2-ol The product was separated by Prep SFC (Stationary phase: Chiralpak Daicel IG 20 × 250 mm, Mobile phase: CO₂, EtOH + 0.4 iPrNH₂). The first fraction was collected as Compound 365a & the second fraction as Compound 365b. |
| | 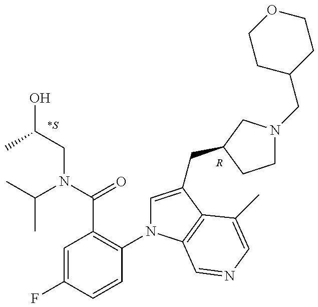 | |

### LCMS (Liquid Chromatography/Mass Spectrometry)

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector.

TABLE 1a
LCMS Method codes (Flow expressed in mL/min; column temperature (T) in ° C.;
Run time in minutes). “TFA” means trifluoroacetic acid; “FA” means formic acid

| | | | | | Flow | |
|---|---|---|---|---|---|---|
| | | | | | (ml/min) | |
| | | | | | - - - | Run |
| Method | | | Mobile | | Column | time |
| code | Instrument | Column | phase | Gradient | T (° C.) | (min) |
| 1 | Agilent | Xbridge C18, | A: 0.02% | 95% A for 0.50 min, to | 1.5 | 6.5 |
| | Techno- | 5 um | CH₃COONH₄; | 5% A in 4.00 min, held for | - - - | |
| | logies 1200 | 4.6*50 mm | B: CH₃CN | 1.50 min, back to 95% A in | 40 | |
| | Series, | | | 0.10 min, held for 0.40 | | |
| | G6110A | | | min | | |
| 2 | Agilent | Xbridge C18, | A: 0.05% | 95% A for 0.50 min, to | 1.5 | 6.5 |
| | Techno- | 5 um | TFA; | 50% A in 4.00 min, then | - - - | |
| | logies 1200 | 4.6*50 mm | B: CH₃CN | to 5% A in 0.50 min, held | 40 | |
| | Series, | | | for 1.00 min, back to 95% | | |
| | G6110A | | | A in 0.10 min, held for | | |
| | | | | 0.40 min | | |
| 3 | Agilent | Xbridge C18, | A: 0.05% | 95% A for 0.50 min, to | 1.5 | 6.5 |
| | Techno- | 5 um | TFA; | 5% A in 4.00 min, held for | - - - | |
| | logies 1200 | 4.6*50 mm | B: CH₃CN | 1.50 min, back to 95% A | 40 | |
| | Series, | | | in 0.10 min, held for 0.40 | | |
| | G6110A | | | min | | |
| 4 | Agilent | Xbridge C18, | A: 0.05% | 90% A for 0.50 min, to | 1.5 | 6.5 |
| | Techno- | 5 um | TFA; B: | 70% A in 4.00 min, then | - - - | |
| | logies 1200 | 4.6*50 mm | CH₃CN | to 5% A in 0.50 min, held | 40 | |
| | Series, | | | for 1.00 min, back to 90% | | |
| | G6110A | | | A in 0.10 min, held for | | |
| | | | | 0.40 min | | |
| 5 | Agilent | Xbridge C18, | A: 0.05% | 98% A for 0.50 min, to | 1.5 | 6.5 |
| | Techno- | 5 um | TFA; B: | 60% A in 4.00 min, then | - - - | |
| | logies 1200 | 4.6*50 mm | CH₃CN | to 5% A in 0.50 min, held | 40 | |
| | Series, | | | for 1.00 min, back to 98% | | |
| | G6110A | | | A in 0.10 min, held for | | |
| | | | | 0.40 min | | |
| 6 | Agilent | Xbridge C18, | A: 0.05% | 95% A for 0.50 min, to | 1.5 | 6.5 |
| | Techno- | 5 um | TFA; B: | 5% A in 4.00 min, held for | - - - | |
| | logies 1200 | 4.6*50 mm | CH₃CN | 1.50 min, back to 95% A | 40 | |
| | Series, | | | in 0.10 min, held for 0.40 | | |
| | G6130A | | | min | | |
| 7 | Agilent | Xbridge C18, | A: 0.05% | 95% A for 0.50 min, to | 1.5 | 6.5 |
| | Techno- | 5 um | TFA; B: | 50% A in 4.00 min, then | - - - | |
| | logies 1200 | 4.6*50 mm | CH₃CN | to 5% A in 0.50 min, held | 40 | |
| | Series, | | | for 1.00 min, back to 95% | | |
| | G6130A | | | A in 0.10 min, held for | | |
| | | | | 0.40 min | | |
| 8 | Agilent | ZORBAX | A: 0.05% | 90% A for 3.00 min, to | 1.5 | 15 |
| | Techno- | SB-C8, | TFA; B: | 5% A in 8.00 min, held for | - - - | |
| | logies 1200 | 3.5 um | CH₃CN | 3.60 min, back to 90% A | 40 | |
| | Series, | 4.6*150 mm | | in 0.10 min, held for 0.30 | | |
| | G6110A | | | min. | | |
| 9 | Agilent | Xbridge C18, | A: 0.05% | 95% A for 0.50 min, to | 1.5 | 6.5 |
| | Techno- | 5 um | TFA; B: | 40% A in 4.00 min, then | - - - | |
| | logies 1200 | 4.6*50 mm | CH₃CN | to 5% A in 0.50 min, held | 40 | |
| | Series, | | | for 1.00 min, back to 95% | | |
| | G6110A | | | A in 0.10 min, held for | | |
| | | | | 0.40 min. | | |
| 10 | Agilent | Waters | mobile phase | First, 90% A was held for | 0.8 | 10 |
| | 1200-6100 | XBridge | A: H₂O with | 0.8 min. Then a gradient | - - - | |
| | | C18, | 0.04% TFA; | was applied to 20% A and | 50 | |
| | | (2.0 × 50 mm, | mobile phase B: | 80% B in 3.7 min and held | | |
| | | 5 uM) | ACN with | for 3 min. And then return | | |
| | | | 0.02% TFA | to 90% A in 2 min and | | |
| | | | | held for 0.5 min. The post | | |
| | | | | time is 0.5 min | | |
| 11 | Agilent | Agilent | mobile phase | a gradient condition from | 1.5 | 3.0 |
| | Prime- | Poroshell | A: | 95% A, 5% B to 20% A, | - - - | |
| | 6125B | 120 HPH- | water(4 L) + N | 80% B in 1.2 min, then to | 30 | |
| | | C18 1.9 um | H₃•H₂O(2.0 | 5% A, 95% B in 1.3 min, | | |
| | | 3.0*30 mm | mL); mobile | and then to 95% A, 5% B | | |
| | | | phase B: | in 0.01 min and held for | | |
| | | | acetonitrile(4 | 0.49 min | | |
| | | | L) | | | |
| 12 | Agilent | Agilent | mobile phase | a gradient condition from | 1.5 | 3.0 |
| | Prime- | Poroshell | A: | 95% A, 5% B to 20% A, | - - - | |
| | 6125B | 120 EC-C18 | water(4 L) + T | 80% B in 1.2 min, then to | 50 | |
| | | 1.9 um | FA(1.5 mL) | 5% A, 95% B in 1.3 min, | | |
| | | 3.0*30 mm | | and then to 95% A, 5% B | | |
| | | | | in 0.01 min and held for | | |
| | | | | 0.49 min | | |
| 13 | Waters: H- | Waters: | A: 0.1% | Gradient start from 5% of | 0.8 | 3.0 |
| | Class + | ACQUITY | NH₄OH in | B increase to 95% within | - - - | |
| | Zspray | UPLC BEH | water, B: | 1.5 min and keep at 95% | 40 | |
| | | C18 (1.7 μm, | CH₃CN | till 2.5 min, then decrease | | |
| | | 2.1 × 30 mm) | | to 5% within 0.01 min and | | |
| | | | | keep at 5% till 3.0 min | | |
| 14 | Agilent: | Waters: | A: 0.1% FA | Gradient start from 5% of | 1.2 | 3.5 |
| | 1260 | Sunfire C18 | solution in | B increase to 95% within | - - - | |
| | Infinity and 6120 | (2.5 μm, | water; B: | 2.5 min and keep at 95% | 50 | |
| | Quadrupole LC/MS | 3.0 × 30 mm) | CH₃CN | till 3.5 min | | |
| 15 | Waters: | Waters: | A: 10 mM | From 100% A to 5% A | 0.6 | 3.5 |
| | Acquity ® | BEH | NH₄HCO₃ in | in 2.10 min, to 0% A in 0.9 | - - - | |
| | UPLC ®- | (1.8 μm, | 95% H₂O + | min, to 5% A in 0.5 min | 55 | |
| | DAD and | 2.1*100 mm) | 5% CH₃CN; | | | |
| | SQD | | B: CH₃CN | | | |
| 16 | Waters: | Waters: | A: 10 mM | From 100% A to 5% A in | 0.6 | 3.5 |
| | Acquity ® | BEH | NH₄HCO₃ in | 2.10 min, to 0% A in 0.9 | - - - | |
| | UPLC ® - | (1.8 μm, | 95% H₂O + | min, to 5% A in 0.5 min | 55 | |
| | DAD and | 2.1*100 mm) | 5% CH₃CN; | | | |
| | SQD and | | B: CH₃CN | | | |
| | ELSD | | | | | |
| 17 | Waters: | Waters: | A: 10 mM | From 100% A to 5% A in | 0.6 | 3.5 |
| | Acquity ® | BEH | NH₄HCO₃ in | 2.10 min, to 0% A in 0.9 | - - - | |
| | UPLC ®- | (1.8 μm, | 95% H₂O + | min, to 5% A in 0.5 min | 55 | |
| | DAD and | 2.1*100 mm) | 5% CH₃CN; | | | |
| | SQD | | B: CH₃CN | | | |
| 18 | Agilent | Xbridge C18, | A: 0.02% | | 1.5 | 6.5 |
| | Technologies | 3.5 um | CH₃COONH₄; | 95% A for 0.50 min, to | - - - | |
| | 1200 | 4.6*50 mm | B: CH₃CN | 5% A in 4.00 min, held for | 40 | |
| | Series, | | | 1.50 min, back to 95% A | | |
| | G6110A | | | in 0.10 min, held for 0.40 | | |
| | | | | min. | | |
| 19 | Agilent | Xbridge C18, | A: 0.02% | 95% A for 0.50 min, to | 1.5 | 6.5 |
| | Technologies | 3.5 um 4.6*50 mm | CH₃COONH₄; | 5% A in 4.00 min, held for | - - - | |
| | 1200 | | B: CH₃CN | 1.50 min, back to 95% A | 40 | |
| | Series, | | | in 0.10 min, held for 0.40 | | |
| | G6130A | | | min. | | |
| 20 | Agilent | Xbridge C18, | A: 0.02% | 95% A for 0.50 min, to | 1.5 | 6.5 |
| | Technologies | 5 um 4.6*50 mm | CH₃COONH₄; | 40% A in 4.00 min, then | - - - | |
| | 1200 | | B: CH₃CN | to 5% A in 0.50 min, held | 40 | |
| | Series, | | | for 1.00 min, back to 95% | | |
| | G6110A | | | A in 0.10 min, held for | | |
| | | | | 0.40 min. | | |
| 21 | Agilent | Xbridge C18, | A: 0.02% | 85% A for 0.50 min, to | 1.5 | 6.5 |
| | Technologies 1200 | 5 um 4.6*50 mm | CH₃COONH₄; | 60% A in 4.00 min, then | - - - | |
| | Series, | | B: CH₃CN | to 5% A in 0.50 min, held | 40 | |
| | G6110A | | | for 1.00 min, back to 85% | | |
| | | | | A in 0.10 min, held for | | |
| | | | | 0.40 min. | | |
| 22 | Agilent | Xbridge C18, | A: 0.05% | 95% A for 0.50 min, to | 1.5 | 6.5 |
| | Technologies 1200 | 3.5 um 4.6*50 mm | TFA; B: | 5% A in 4.00 min, held for | - - - | |
| | Series, | | CH₃CN | 1.50 min, back to 95% A | 40 | |
| | G6130A | | | in 0.10 min, held for 0.40 | | |
| | | | | min | | |
| 23 | Agilent | Xbridge C18, | A: 0.02% | 95% A for 0.50 min, to | 1.5 | 6.5 |
| | Technologies 1200 | 3.5 um 4.6*50 mm | CH₃COONH₄; | 5% A in 4.00 min, held for | - - - | |
| | Series, | | B: CH₃CN | 1.50 min, back to 95% A | 40 | |
| | G6130A | | | in 0.10 min, held for 0.40 | | |
| | | | | min. | | |
| 24 | Agilent | Xbridge C18, | A: 0.02% | 80% A for 0.50 min, to | 1.5 | 6.5 |
| | Technologies 1200 | 3.5 um 4.6*50 mm | CH₃COONH₄; | 40% A in 4.00 min, then | - - - | |
| | Series, | | B: CH₃CN | to 5% A in 0.50 min, held | 40 | |
| | G6130A | | | for 1.00 min, back to 80% | | |
| | | | | A in 0.10 min, held for | | |
| | | | | 0.40 min. | | |
| 25 | Agilent | Xbridge C18, | A: 0.02% | 90% A for 0.50 min, to | 1.5 | 6.5 |
| | Technologies 1200 | 5 um 4.6*50 mm | CH₃COONH₄; | 50% A in 4.00 min, then | - - - | |
| | Series, | | B: CH₃CN | to 5% A in 0.50 min, held | 40 | |
| | G6130A | | | for 1.00 min, back to 90% | | |
| | | | | A in 0.10 min, held for | | |
| | | | | 0.40 min. | | |
| 26 | Agilent | Xbridge C18, | A: 0.05% | 85% A for 0.50 min, to | 1.5 | 6.5 |
| | Technologies 1200 | 5 um 4.6*50 mm | TFA; B: | 70% A in 4.00 min, then | - - - | |
| | Series, | | CH₃CN | to 5% A in 0.50 min, held | 40 | |
| | G6110A | | | for 1.00 min, back to 85% | | |
| | | | | A in 0.10 min, held for | | |
| | | | | 0.40 min. | | |
| 27 | Agilent | Xbridge C18, | A: 0.02% | 95% A for 0.50 min, to | 1.5 | 6.5 |
| | Technologies 1200 | 5 um 4.6*50 mm | CH₃COONH₄; | 5% A in 4.00 min, held for | - - - | |
| | Series, | | B: CH₃CN | 1.50 min, back to 95% A | 40 | |
| | G6130A | | | in 0.10 min, held for 0.40 | | |
| | | | | min. | | |
| 28 | Agilent | Xbridge C18, | A: 0.02% | 95% A for 0.50 min, to | 1.5 | 6.5 |
| | Technologies 1200 | 5 um 4.6*50 mm | CH₃COONH₄; | 40% A in 4.00 min, then | - - - | |
| | Series, | | B: CH₃CN | to 5% A in 0.50 min, held | 40 | |
| | G6130A | | | for 1.00 min, back to 95% | | |
| | | | | A in 0.10 min, held for | | |
| | | | | 0.40 min. | | |
| 29 | Agilent | Xbridge C18, | A: 0.02% | 95% A for 0.50 min, to | 1.5 | 6.5 |
| | Technologies 1200 | 5 um 4.6*50 mm | CH₃COONH₄; | 50% A in 4.00 min, then | - - - | |
| | Series, | | B: CH₃CN | to 5% A in 0.50 min, held | 40 | |
| | G6110A | | | for 1.00 min, back to 95% | | |
| | | | | A in 0.10 min, held for | | |
| | | | | 0.40 min. | | |
| 30 | Waters: | Waters: | A: 10 mM | From 100% A to 5% A in | 0.6 | 3.5 |
| | Acquity ® | BEH | CH₃COONH₄ | 2.10 min, to 0% A in | - - - | |
| | UPLC ®- | (1.7 μm, | in 95% H₂O + 5% | 0.9 min, to 5% A in | 55 | |
| | DAD and | 2.1*100 mm) | CH₃CN | 0.5 min | | |
| | SQD | | B: CH₃CN | | | |
| 31 | Waters: | Waters: | A: 10 mM | From 100% A to 5% A | 0.6 | 3.5 |
| | Acquity ® | BEH | NH₄HCO₃ in | in 2.10 min, to 0% A in 0.9 | - - - | |
| | UPLC ®- | (1.7 μm, | 95% H₂O + | min, to 5% A in 0.5 min | 5 | |
| | DAD and | 2.1*100 mm) | 5% CH₃CN | | | |
| | SQD | | B: CH₃CN | | | |
| 32 | Waters: | Waters: | A: 0.1% | From 95% A to | 0.3 | 28 |
| | Acquity ® | BEH | NH₄HCO₃ | 5% A in 20 min, hold 3 | - - - | |
| | UPLC ®- | (1.7 μm, | in 95% H₂O + 5% | min, | 45 | |
| | DAD and | 2.1*150 mm) | CH₃CN | to 95% A in 1 min, | | |
| | SQD | | B: CH₃CN | hold 4 min | | |
| 33 | Agilent | XBridge | A: 10 mM | From 95% A to 20% A in | 1.0 | 30 |
| | Technologies | C18, 3.5 | NH₄HCO₃ in | 20 min, hold 2 min, to 95% | - - - | |
| | 1260 | um, 4.6 × | water pH 9.5 | A in 1 min, hold 7 min | 45 | |
| | Series | 150 mm | B: CH₃CN | | | |
| 34 | Waters: | Waters: | A: 10 mM | From 100% A to | 0.6 | 3.5 |
| | Acquity ® | BEH | CH₃COONH₄ | 5% A in 2.10 min, | - - - | |
| | UPLC ®- | (1.7 μm, | in 95% H₂O + | to 0% A in 0.90 min, | 55 | |
| | DAD and | 2.1*100 mm) | 5% CH₃CN | to 5% A in 0.5 min | | |
| | SQD | | B: CH₃CN | | | |
| 35 | Waters: | Waters: | A: 0.1% | From 100% A to | 0.8 | 2.0 |
| | Acquity | BEH | NH₄HCO₃ | 5% A in 1.3 min, | - - - | |
| | UPLC ®- | (1.7 μm, | in 95% H₂O + | hold 0.7 min | 55 | |
| | DAD and | 2.1*50 mm) | 5% CH₃CN | | | |
| | SQD | | B: CH₃CN | | | |
| 36 | Waters: | Waters: | A: 10 mM | From 95% A to | 0.8 | 2.0 |
| | Acquity ® | BEH | CH₃COONH₄ | 5% A in 1.3 min, | - - - | |
| | UPLC ® - | (1.7 μm, | in 95% H₂O + | held for 0.7 min | 55 | |
| | DAD and | 2.1*50 mm) | 5% CH₃CN | | | |
| | SQD | | B: CH₃CN | | | |
| 37 | SHIMADZ | MERCK, | A:water(4 L) + | a gradient condition from | 1.5 | 1.5 |
| | U LC20- | RP-18e 25- | TFA (1.5 mL) | 95% A, 5% B to 5% A, | - - - | |
| | MS2010 | 2 mm | B: aceto- | 95% B in 0.7 minutes, | 50 | |
| | | | nitrile (4 L) + | hold at these conditions | | |
| | | | TFA | for 0.4 minutes, and then | | |
| | | | (0.75 mL) | to 95% A, 5% B in 0.01 | | |
| | | | | min and held for 0.49 min. | | |
| 38 | Agilent | Xtimate C18 | A: water(4 L) ) + | a gradient condition from | 1.2 | 2.0 |
| | LC1200- | 2.1*30 mm, 3 | TFA (1.5 mL) | 90% A, 10% B to 20% A, | - - - | |
| | MS6110 | um | B: aceto- | 80% B in 0.9 minutes, and | 50 | |
| | | | nitrile(4 L) + T | hold at these conditions | | |
| | | | FA (0.75 mL) | for 0.6 minutes, then to | | |
| | | | | 90% A, 10% B in 0.01 | | |
| | | | | min, and held for 0.49 | | |
| | | | | min. | | |
| 39 | SHIMADZ | Xbridge | A: water(4 L) + | a gradient condition from | 0.8 | 7.0 |
| | U LC20- | Shield RP- | NH₃•H₂O | 90% A to 20% A, 80% B | - - - | |
| | MS2020 | 18, 5 um, 2.1* | (0.8 mL) | in 6 minutes, and hold at | 50 | |
| | | 50 mm | B: aceto- | these conditions for 0.5 | | |
| | | | nitrile | minutes, then to 90% A | | |
| | | | | and 10% B in 0.1 min and | | |
| | | | | held for 0.49 min. | | |
| 40 | SHIMADZ | Xbridge | A: water(4 L) + | a gradient condition from | 0.8 | 7.0 |
| | U LC20- | Shield RP- | NH₃•H₂O | 70% A to 10% A, 90% B | - - - | |
| | MS2020 | 18, 5 um, 2.1* | (0.8 mL) | in 6 minutes, and hold at | 50 | |
| | | 50 mm | B: aceto- | these conditions for 0.5 | | |
| | | | nitrile | minutes, then to 70% A | | |
| | | | | and 30% B in 0.1 min and | | |
| | | | | held for 0.49 min. | | |

TABLE 1b
LCMS and melting point data. Co. No. means compound
number; Rₜ means retention time in min.

| Co. No. | Rt | [M + 1]⁺ | LCMS method |
|---|---|---|---|
| 1 | 2.81 | 594.5 | 9 |
| 2 | 2.81 | 594.5 | 9 |
| 2a | 2.89 | 594.6 | 2 |
| 3 | 1.11 | 614.5 | 12 |
| 4 | 1.11 | 614.5 | 12 |
| 4a | 1.12 | 614.5 | 12 |
| 4b | 1.11 | 614.5 | 12 |
| 6 | 1.67 | 598.9 | 13 |
| 7 | 1.33 | 648.3 | 14 |
| 8 | 3.02 | 633.5 | 4 |
| 8a | 1.70 | 633.5 | 11 |
| 8b | 1.00 | 633.5 | 12 |
| 9 | 3.04 | 633.5 | 4 |
| 9a | 1.00 | 633.5 | 12 |
| 9a | 0.98 | 633.5 | 12 |
| 9b | 1.00 | 633.5 | 12 |
| 10 | 2.99 | 668.6 | 1 |
| 11 | 2.65 | 668.6 | 9 |
| 12 | 2.76 | 659.5 | 2 |
| 13 | 2.26 | 659.6 | 3 |
| 14 | 3.02 | 580.6 | 2 |
| 15 | 2.88 | 580.5 | 2 |
| 16 | 3.20 | 645.5 | 2 |
| 17 | 2.79 | 645.6 | 2 |
| 18a | 1.09 | 679.5 | 12 |
| 18b | 1.08 | 679.5 | 12 |
| 19a | 1.09 | 679.5 | 12 |
| 19b | 1.09 | 679.5 | 12 |
| 20 | 2.87 | 671.3 | 2 |
| 21 | 3.10 | 671.3 | 4 |
| 22 | 2.70 | 657.6 | 2 |
| 23 | 2.23 | 657.5 | 6 |
| 24 | 2.73 | 631.3 | 2 |
| 25 | 2.74 | 631.3 | 2 |
| 26a | 1.07 | 677.5 | 12 |
| 26b | 1.07 | 677.5 | 12 |
| 27a | 1.69 | 677.5 | 11 |
| 27b | 1.75 | 677.5 | 11 |
| 27c | 1.69 | 677.5 | 11 |
| 27d | 1.75 | 677.5 | 11 |
| 28 | 2.60 | 643.5 | 2 |
| 28b | 2.62 | 643.6 | 2 |
| 30 | 3.00 | 629.3 | 5 |
| 31 | 2.23 | 629.6 | 6 |
| 32a | 2.83 | 631.3 | 2 |
| 32b | 3.02 | 631.2 | 4 |
| 33a | 2.31 | 666.2 | 3 |
| 33b | 2.31 | 666.2 | 3 |
| 33c | 2.96 | 666.2 | 2 |
| 34a | 2.91 | 667.2 | 2 |
| 34b | 2.92 | 667.2 | 2 |
| 35a | 2.91 | 645.3 | 2 |
| 35b | 2.91 | 645.3 | 2 |
| 36a | 2.99 | 680.5 | 7 |
| 36b | 2.97 | 680.5 | 7 |
| 37a | 3.05 | 681.5 | 7 |
| 37b | 3.05 | 681.5 | 7 |
| 38a | 2.89 | 671.6 | 7 |
| 38b | 2.92 | 671.6 | 7 |
| 39a | 1.08 | 677.5 | 12 |
| 39b | 1.09 | 677.5 | 12 |
| 40a | 1.07 | 677.5 | 12 |
| 40b | 1.08 | 677.5 | 12 |
| 41 | 0.85 | 568.3 | 10 |
| 42 | 2.85 | 507.4 | 1 |
| 43 | 3.05 | 507.4 | 4 |
| 44 | 0.96 | 465.3 | 14 |
| 45 | 1.57 | 465.7 | 13 |
| 46 | 1.23 | 479.7 | 13 |
| 47 | 1.23 | 479.8 | 13 |
| 48 | 3.05 | 521.2 | 2 |
| 49 | 3.03 | 521.2 | 2 |
| 49a | 1.081 | 521.4 | 12 |
| 49b | 1.081 | 521.4 | 12 |
| 50 | 1.29 | 548.8 | 13 |
| 51 | 7.28 | 548.2 | 8 |
| 58 | 1.33 | 562.8 | 13 |
| 59 | 1.29 | 578.9 | 13 |
| 60 | 1.31 | 573.9 | 13 |
| 61 | 1.57 | 493.4 | 15 |
| 62 | 2.00 | 592.5 | 15 |
| 63 | 1.50 | 534.4 | 16 |
| 64 | 1.61 | 570.4 | 16 |
| 65 | 1.70 | 550.4 | 16 |
| 66 | 1.01 | 564.4 | 12 |
| 67 | 1.01 | 559.4 | 12 |
| 68 | 1.64 | 598.4 | 11 |
| 69 | 1.63 | 598.4 | 11 |
| 70 | 1.55 | 562.4 | 11 |
| 71 | 1.55 | 562.4 | 11 |
| 72 | 1.05 | 562.4 | 12 |
| 73 | 1.04 | 562.4 | 12 |
| 74 | 1.48 | 548.5 | 11 |
| 75 | 1.64 | 613.4 | 15 |
| 76 | 1.62 | 625.5 | 15 |
| 77 | 1.81 | 527.4 | 17 |
| 77a | 1.73 | 527.5 | 15 |
| 77b | 1.73 | 527.5 | 15 |
| 77c | 1.73 | 527.5 | 15 |
| 77d | 1.73 | 527.5 | 15 |
| 78 | 1.52 | 513.4 | 15 |
| 78a | 1.57 | 513.5 | 15 |
| 78b | 1.56 | 513.5 | 15 |
| 78c | 1.56 | 513.5 | 15 |
| 78d | 1.57 | 513.5 | 15 |
| 79 | 1.55 | 513.4 | 15 |
| 80 | 1.41 | 499.4 | 15 |
| 81 | 1.14 | 534.78 | 13 |
| 82 | 1.16 | 578.84 | 13 |
| 83 | 1.48 | 522.4 | 16 |
| 84 | 2.681 | 548.2 | 1 |
| 85 | 3.399 | 578.2 | 1 |
| 86 | 2.771 | 578.2 | 1 |
| 87 | 2.734 | 564.2 | 1 |
| 88 | 1.11 | 520.82 | 13 |
| 89 | 1.21 | 520.85 | 13 |
| 90a | 1.6 | 534.6 | 15 |
| 90b | 1.6 | 534.6 | 15 |
| 91 | 1.92 | 604.5 | 16 |
| 92 | 1.69 | 549.4 | 16 |
| 93 | 1.72 | 555.4 | 17 |
| 94 | 1.49 | 520.5 | 15 |
| 95 | 3.774 | 506.4 | 1 |
| 96 | 1.67 | 542.4 | 17 |
| 97 | 1.67 | 518.4 | 17 |
| 98 | 1.73 | 520.4 | 17 |
| 99 | 1.15 | 534.89 | 13 |
| 100 | 1.15 | 534.89 | 13 |
| 101 | 2.739 | 563.2 | 1 |
| 102 | 2.579 | 534.2 | 1 |
| 103 | 1.44 | 508.4 | 11 |
| 104 | 1.45 | 520.5 | 16 |
| 105 | 2.989 | 520.1 | 1 |
| 106 | 2.858 | 652.1 | 1 |
| 107 | 0.9 | 560.3 | 13 |
| 108 | 2.77 | 652.2 | 1 |
| 109 | 2.435 | 521.4 | 1 |
| 110 | 2.220 | 520.1 | 1 |
| 111 | 2.262 | 564.2 | 1 |
| 112 | 2.644 | 548.1 | 1 |
| 113 | 2.645 | 548.2 | 1 |
| 114 | 1.36 | 506.4 | 30 |
| 116a | 1.589 | 562.4 | 12 |
| 116b | 1.503 | 564.4 | 12 |
| 117a | 1.065 | 592.5 | 12 |
| 117b | 1.068 | 592.5 | 12 |
| 118 | 1.044 | 548.4 | 11 |
| 119a | 1.01 | 508.4 | 12 |
| 119b | 0.998 | 508.4 | 12 |
| 120 | 2.536 | 506.1 | 1 |
| 121 | 3.083 | 576.2 | 1 |
| 122 | 3.068 | 562.2 | 1 |
| 123 | 2.954 | 578.2 | 1 |
| 124 | 1.38 | 556.09 | 13 |
| 125 | 1.36 | 593.06 | 13 |
| 126 | 1.52 | 602.07 | 13 |
| 127 | 1.36 | 546.4 | 30 |
| 128 | 1.58 | 568.4 | 16 |
| 128a | 1.6 | 568.5 | 15 |
| 128b | 1.6 | 568.5 | 15 |
| 128c | 1.6 | 568.5 | 15 |
| 128d | 1.6 | 568.5 | 15 |
| 129 | 7.343 | 548.2 | 1 |
| 130 | 1.76 | 562.6 | 15 |
| 131 | 1.8 | 562.6 | 15 |
| 132 | 1.8 | 562.6 | 15 |
| 133 | 1.76 | 562.6 | 15 |
| 134 | 1.45 | 507.6 | 15 |
| 134a | 1.44 | 507.5 | 15 |
| 134b | 1.41 | 507.5 | 15 |
| 134c | 1.44 | 507.5 | 15 |
| 134d | 1.44 | 507.5 | 15 |
| 135 | 2.986 | 574.2 | 1 |
| 136 | 2.813 | 578.2 | 1 |
| 137a | 1.31 | 548.93 | 13 |
| 137b | 1.4 | 548.83 | 13 |
| 138 | 1.85 | 507.4 | 16 |
| 138a | 1.87 | 507.5 | 15 |
| 138b | 1.88 | 507.3 | 15 |
| 139 | 1.45 | 520.4 | 16 |
| 139a | 1.52 | 520.5 | 17 |
| 139b | 1.52 | 520.5 | 17 |
| 140 | 1.535 | 562.5 | 11 |
| 141 | 1.4 | 566.98 | 13 |
| 142 | 1.589 | 562.5 | 11 |
| 143 | 1.562 | 562.6 | 11 |
| 144 | 1.45 | 507.4 | 16 |
| 145 | 1.59 | 562.5 | 17 |
| 146 | 1.66 | 562.4 | 30 |
| 147a | 1.83 | 507.5 | 15 |
| 147b | 1.83 | 507.5 | 15 |
| 148 | 3.885 | 506.4 | 1 |
| 149 | 2.493 | 577.2 | 1 |
| 150 | 1.046 | 548.4 | 11 |
| 151 | 1.03 | 548.4 | 11 |
| 152 | 2.979 | 589.5 | 1 |
| 153 | 1.97 | 563.4 | 16 |
| 154 | 1.96 | 563.4 | 16 |
| 155 | 1.531 | 587.4 | 11 |
| 156 | 1.16 | 534.87 | 13 |
| 157 | 3.635 | 589.5 | 2 |
| 158 | 1.5 | 592.4 | 11 |
| 159 | 1.546 | 574.4 | 11 |
| 160 | 1.83 | 576.5 | 30 |
| 161 | 1.37 | 571.09 | 13 |
| 162 | 1.071 | 562.4 | 11 |
| 163 | 1.068 | 562.3 | 11 |
| 164a | 1.089 | 562.4 | 12 |
| 164b | 1.088 | 562.4 | 12 |
| 165a | 1.61 | 551.5 | 15 |
| 165b | 1.62 | 551.6 | 15 |
| 166a | 1.59 | 513.5 | 15 |
| 166b | 1.59 | 513.5 | 15 |
| 167a | 1.45 | 499.5 | 15 |
| 167b | 1.45 | 499.5 | 15 |
| 168 | 2.628 | 563.2 | 2 |
| 169 | 1.51 | 534.4 | 16 |
| 169a | 1.54 | 534.5 | 16 |
| 169b | 1.49 | 534.5 | 16 |
| 170 | 2.853 | 577.2 | 2 |
| 171 | 2.826 | 534.1 | 3 |
| 172 | 1.48 | 621.9 | 13 |
| 173 | 1.51 | 593.95 | 13 |
| 174 | 1.42 | 619.8 | 13 |
| 175 | 1.003 | 534.3 | 11 |
| 176 | 1.005 | 534.3 | 11 |
| 177 | 1.01 | 564.4 | 11 |
| 178 | 1.52 | 588.4 | 30 |
| 179 | 1.65 | 588.5 | 16 |
| 180a | 1.59 | 527.5 | 15 |
| 180b | 1.6 | 527.5 | 15 |
| 181a | 1.57 | 527.3 | 16 |
| 181b | 1.56 | 527.4 | 16 |
| 182a | 1.31 | 527.5 | 15 |
| 182b | 1.31 | 527.5 | 15 |
| 183a | 1.6 | 527.5 | 15 |
| 183b | 1.6 | 527.5 | 15 |
| 184a | 1.63 | 548.5 | 15 |
| 184b | 1.63 | 548.6 | 15 |
| 184 | 1.6 | 548.4 | 16 |
| 185 | 1.54 | 562.4 | 16 |
| 186a | 3.449 | 520.1 | 26 |
| 186b | 2.803 | 520.1 | 27 |
| 187 | 3.634 | 546.1 | 3 |
| 188a | 1.35 | 548.88 | 13 |
| 188b | 1.44 | 548.67 | 13 |
| 189 | 1.41 | 562.5 | 17 |
| 190a | 1.73 | 493.5 | 16 |
| 190b | 1.73 | 493.5 | 15 |
| 191a | 1.43 | 479.5 | 17 |
| 191b | 1.42 | 479.5 | 17 |
| 192 | 1.47 | 547.3 | 16 |
| 193 | 2.2 | 505.97 | 13 |
| 194 | 2.22 | 505.95 | 13 |
| 195 | 1.3 | 534.88 | 13 |
| 196 | 2.977 | 506.1 | 6 |
| 197 | 1.056 | 557.4 | 12 |
| 198a | 1.57 | 537.4 | 16 |
| 198b | 1.57 | 537.4 | 16 |
| 199 | 2.569 | 506.2 | 8 |
| 200 | 2.708 | 548.1 | 9 |
| 201 | 2.758 | 548.2 | 9 |
| 202 | 1.37 | 577.97 | 13 |
| 203 | 1.55 | 607.9 | 13 |
| 204 | 1.46 | 633.9 | 13 |
| 205 | 1.61 | 521.5 | 17 |
| 205a | 1.62 | 521.5 | 17 |
| 205b | 1.63 | 521.5 | 17 |
| 206a | 1.33 | 578.5 | 15 |
| 206b | 1.32 | 578.6 | 15 |
| 207 | 1.3 | 549.95 | 13 |
| 208 | 3.334 | 546.2 | 9 |
| 209 | 3.370 | 546.1 | 9 |
| 210 | 2.685 | 548.2 | 9 |
| 211 | 2.734 | 548.2 | 9 |
| 212 | 2.588 | 520.1 | 9 |
| 213a | 1.483 | 555.3 | 12 |
| 213b | 1.481 | 555.3 | 12 |
| 214 | 1.37 | 552.4 | 16 |
| 214a | 1.42 | 552.4 | 16 |
| 214b | 1.37 | 552.4 | 16 |
| 215 | 1.29 | 534.88 | 13 |
| 216 | 2.763 | 548.2 | 19 |
| 217 | 1.38 | 563.87 | 13 |
| 218 | 1.39 | 563.8 | 13 |
| 219 | 1.27 | 549.7 | 13 |
| 220 | 2.755 | 546.1 | 20 |
| 221 | 1.648 | 562.4 | 12 |
| 222 | 2.796 | 548.2 | 20 |
| 223 | 3.000 | 546.1 | 21 |
| 224 | 1.24 | 552.4 | 30 |
| 224a | 1.24 | 552.4 | 30 |
| 224b | 1.23 | 552.4 | 30 |
| 225 | 1.55 | 619.4 | 31 |
| 226 | 1.63 | 562.5 | 16 |
| 227 | 1.7 | 562.6 | 30 |
| 228 | 1.65 | 562.5 | 16 |
| 229 | 1.67 | 562.5 | 16 |
| 230a | 1.3 | 568.5 | 30 |
| 230b | 1.29 | 568.5 | 30 |
| 231a | 1.3 | 568.5 | 30 |
| 231b | 1.29 | 568.5 | 30 |
| 232a | 1.29 | 568.5 | 30 |
| 232b | 1.29 | 568.5 | 30 |
| 233a | 1.29 | 568.5 | 30 |
| 233b | 1.3 | 568.5 | 30 |
| 234 | 3.200 | 562.2 | 22 |
| 235 | 1.51 | 591.9 | 13 |
| 236 | 1.079 | 562.4 | 12 |
| 237 | 1.47 | 606.85 | 13 |
| 238 | 2.277 | 534.4 | 22 |
| 239 | 2.431 | 521.4 | 22 |
| 240a | 1.73 | 564.6 | 17 |
| 240b | 1.73 | 564.5 | 17 |
| 241 | 1.528 | 548.4 | 12 |
| 242 | 1.535 | 548.4 | 12 |
| 244 | 1.44 | 592.69 | 13 |
| 245 | 1.36 | 590.8 | 13 |
| 246 | 1.104 | 576.5 | 12 |
| 247 | 1.109 | 576.4 | 12 |
| 248 | 1.437 | 564.4 | 12 |
| 249 | 1.41 | 590.61 | 13 |
| 250 | 1.34 | 578.56 | 13 |
| 251 | 1.3 | 576.59 | 13 |
| 252a | 2.618 | 565.5 | 28 |
| 252b | 2.632 | 565.5 | 28 |
| 253a | 2.789 | 551.2 | 29 |
| 253b | 2.778 | 551.1 | 18 |
| 254 | 2.474 | 548.4 | 22 |
| 255 | 2.552 | 562.4 | 22 |
| 256 | 2.770 | 562.5 | 22 |
| 257 | 2.710 | 521.4 | 23 |
| 258 | 2.932 | 576.5 | 23 |
| 259 | 2.840 | 562.5 | 26 |
| 260 | 3.092 | 562.4 | 25 |
| 261 | 0.81 | 517 | 15 |
| 262 | 0.74 | 503 | 16 |
| 263 | 0.68 | 519 | 16 |
| 264 | 0.7 | 519 | 15 |
| 265 | 0.79 | 491 | 15 |
| 266 | 0.67 | 519 | 16 |
| 267 | 0.75 | 535 | 16 |
| 268 | 0.67 | 521 | 16 |
| 269 | 0.69 | 521 | 16 |
| 270 | 0.7 | 522 | 15 |
| 271 | 0.66 | 521 | 16 |
| 272 | 0.68 | 535 | 16 |
| 273 | 0.65 | 519 | 16 |
| 274 | 0.65 | 521 | 16 |
| 275 | 0.64 | 521 | 16 |
| 276 | 0.69 | 535 | 16 |
| 277 | 0.66 | 521 | 16 |
| 278 | 0.67 | 521 | 16 |
| 279 | 0.77 | 505 | 16 |
| 280 | 0.85 | 519 | 15 |
| 281 | 0.69 | 523 | 16 |
| 282 | 0.65 | 521 | 16 |
| 283 | 0.81 | 542 | 16 |
| 284 | 0.76 | 501 | 16 |
| 285 | 0.73 | 489 | 16 |
| 286 | 0.79 | 479 | 16 |
| 287 | 0.74 | 479 | 16 |
| 288 | 0.76 | 479 | 16 |
| 289 | 0.78 | 493 | 16 |
| 290 | 0.73 | 506 | 16 |
| 291 | 0.76 | 520 | 16 |
| 292a | 0.83 | 534 | 16 |
| 292b | 0.77 | 534 | 16 |
| 293a | 0.83 | 534 | 16 |
| 293b | 0.77 | 534 | 16 |
| 294a | 0.72 | 546 | 16 |
| 294b | 0.73 | 546 | 16 |
| 295 | 0.73 | 534 | 16 |
| 296 | 0.81 | 560 | 16 |
| 297 | 0.87 | 575 | 15 |
| 298 | 0.83 | 575 | 15 |
| 299 | 0.84 | 549 | 15 |
| 300 | 0.78 | 575 | 15 |
| 301 | 0.82 | 549 | 15 |
| 302 | 0.83 | 549 | 15 |
| 303 | 0.75 | 575 | 15 |
| 304 | 0.73 | 547 | 15 |
| 305 | 0.7 | 533 | 15 |
| 306 | 0.75 | 547 | 15 |
| 307 | 0.71 | 510 | 15 |
| 308 | 0.87 | 491 | 15 |
| 309a | 0.85 | 533 | 15 |
| 309b | 0.75 | 533 | 15 |
| 310 | 0.81 | 573 | 15 |
| 311 | 0.74 | 559 | 15 |
| 311a | 1.42 | 558.5 | 17 |
| 311b | 1.41 | 558.5 | 17 |
| 312 | 0.66 | 575 | 15 |
| 313 | 0.68 | 591 | 15 |
| 313a | 1.31 | 590.6 | 17 |
| 313b | 1.32 | 590.6 | 17 |
| 314 | 0.75 | 552 | 15 |
| 315 | 0.75 | 561 | 15 |
| 316 | 0.79 | 575 | 15 |
| 317 | 0.81 | 575 | 15 |
| 318 | 0.74 | 591 | 15 |
| 319 | 0.75 | 561 | 15 |
| 320 | 0.73 | 593 | 15 |
| 320a | 1.39 | 592.6 | 17 |
| 320b | 1.5 | 592.6 | 17 |
| 321 | 0.75 | 591 | 15 |
| 322 | 0.67 | 577 | 15 |
| 323 | 0.67 | 577 | 15 |
| 324 | 0.65 | 577 | 15 |
| 325 | 0.66 | 577 | 15 |
| 326 | 0.69 | 579 | 15 |
| 327 | 0.65 | 577 | 15 |
| 328 | 0.74 | 605 | 16 |
| 329 | 0.74 | 607 | 16 |
| 330 | 0.79 | 621 | 16 |
| 331 | 0.79 | 621 | 16 |
| 332 | 0.72 | 605 | 16 |
| 333 | 0.72 | 605 | 16 |
| 334 | 0.75 | 595 | 16 |
| 335 | 0.79 | 609 | 16 |
| 336 | 0.79 | 609 | 16 |
| 337 | 0.75 | 593 | 16 |
| 338 | 0.8 | 607 | 16 |
| 339 | 0.8 | 607 | 16 |
| 340 | 0.83 | 617 | 15 |
| 341 | 0.73 | 602.5 | 16 |
| 342 | 0.98 | 619 | 15 |
| 343 | 0.96 | 619 | 15 |
| 344 | 0.82 | 619 | 16 |
| 345 | 0.66 | 619 | 16 |
| 346 | 0.76 | 619 | 16 |
| 347 | 0.8 | 630 | 15 |
| 348 | 0.84 | 646 | 15 |
| 349 | 0.71 | 617 | 15 |
| 350 | 0.76 | 633 | 15 |
| 351a | 6.47 | 544.4 | 32 |
| 351b | 7.78 | 544.4 | 32 |
| 352a | 1.4 | 560.6 | 17 |
| 352b | 1.39 | 560.6 | 17 |
| 353a | 1.34 | 546.5 | 17 |
| 353b | 1.34 | 546.5 | 17 |
| 354 | 1.46 | 574.6 | 17 |
| 357 | 0.69 | 537.4 | 32 |
| 358 | 0.65 | 535.4 | 32 |
| 359 | 0.65 | 535.4 | 32 |
| 360 | 0.67 | 535.3 | 32 |
| 361 | 0.68 | 535.4 | 32 |
| 362a | 1.35 | 549.6 | 15 |
| 362b | 1.3 | 549.6 | 15 |
| 363a | 1.53 | 549.5 | 16 |
| 363b | 1.55 | 549.5 | 16 |
| 364a | 1.35 | 549.5 | 17 |
| 364b | 1.34 | 549.5 | 17 |
| 365a | 1.65 | 551.5 | 17 |
| 365b | 1.65 | 551.6 | 17 |
| 366 | 3.53 | 565.2 | 2 |
| 367 | 3.53 | 565.2 | 2 |
| 368 | 3.52 | 565.2 | 2 |
| 369 | 4.94 | 565.4 | 39 |
| 370 | 0.707 | 585.1 | 37 |
| 371 | 0.703 | 585.2 | 37 |
| 372 | 0.693 | 585.2 | 37 |
| 373 | 0.701 | 585.1 | 37 |
| 374 | 0.658 | 585.2 | 37 |
| 375 | 0.658 | 585.1 | 37 |
| 378 | 1.69 | 606.84 | 14 |
| 380 | 1.74 | 620.89 | 14 |
| 383 | 2.11 | 592.5 | 15 |
| 386 | 1.464 | 618.3 | 14 |
| 387 | 0.708 | 620.4 | 37 |
| 388 | 0.708 | 620.4 | 37 |
| 392 | 1.490 | 624.3 | 14 |
| 394 | 2.29 | 606.4 | 15 |
| 395 | 4.560 | 606.5 | 39 |
| 396 | 4.635 | 606.5 | 39 |
| 398 | 0.689 | 592.3 | 37 |
| 399 | 0.693 | 592.3 | 37 |
| 400 | 2.10 | 620.5 | 15 |
| 401 | 2.00 | 606.5 | 15 |
| 402 | 0.758 | 620.5 | 37 |
| 403 | 0.757 | 620.4 | 37 |
| 407 | 1.83 | 563.4 | 15 |
| 414 | 1.97 | 620.5 | 14 |
| 415 | 1.98 | 620.4 | 14 |
| 420 | 0.701 | 606.2 | 37 |
| 421 | 0.699 | 606.2 | 37 |
| 422 | 0.664 | 634.9 | 37 |
| 423 | 0.660 | 634.9 | 37 |
| 428 | 2.754 | 620.5 | 1 |
| 429 | 1.37 | 492.4 | 16 |
| 431 | 1.37 | 492.4 | 15 |
| 433 | 1.12 | 520.5 | 14 |
| 434 | 0.334 | 492.3 | 14 |
| 435 | 0.409 | 492.3 | 14 |
| 437 | 0.609 | 466.3 | 37 |
| 438 | 0.364 | 518.3 | 14 |
| 441 | 0.566 | 506.2 | 37 |
| 442 | 0.321 | 520.3 | 37 |
| 443 | 0.427 | 520.3 | 37 |
| 449 | 0.351 | 506.4 | 40 |
| 444 | 0.609 | 466.3 | 37 |
| 446 | 1.35 | 504.4 | 15 |
| 448 | 0.98 | 506.4 | 35 |
| 452 | 0.595 | 492.2 | 37 |
| 453 | 0.591 | 492.3 | 37 |
| 454 | 0.396 | 522.3 | 38 |
| 456 | 1.41 | 520.4 | 15 |
| 457 | 1.35 | 506.4 | 15 |
| 458 | 0.321 | 520.3 | 37 |
| 459 | 0.427 | 520.3 | 37 |
| 468 | 1.69 | 520.2 | 14 |
| 469 | 1.68 | 520.54 | 14 |
| 486 | 0.665 | 620.3 | 37 |
| 487 | 0.743 | 606.8 | 37 |
| 488 | 0.577 | 520.3 | 37 |
| 489 | 2.24 | 613 | 15 |
| 490 | 1.44 | 513.4 | 15 |
| 491 | 1.487 | 592.3 | 14 |
| 492 | 1.51 | 592.9 | 14 |
| 497 | 1.95 | 563.4 | 15 |
| 498 | 4.209 | 549.4 | 39 |
| 499 | 4.282 | 549.5 | 39 |
| 500 | 4.266 | 622.6 | 39 |
| 503 | 1.33 | 423.3 | 15 |
| 504 | 1.34 | 423.3 | 31 |
| 505 | 1.33 | 423.3 | 15 |
| 507 | 1.33 | 423.3 | 15 |
| 508 | 1.29 | 423.3 | 15 |
| 509 | 1.34 | 423.4 | 34 |
| 510 | 1.34 | 423.4 | 34 |
| 516 | 1.08 | 535.5 | 35 |
| 517 | 1.09 | 523.3 | 35 |
| 519 | 1.51 | 547.5 | 34 |
| 520 | 0.74 | 551 | 36 |
| 521 | 1.76 | 560.5 | 34 |
| 522 | 1.33 | 423.3 | 15 |
| 525 | 0.765 | 640.3 | 37 |
| 526a | 0.681 | 563.3 | 37 |
| 526b | 0.674 | 563.3 | 37 |
| 527 | 0.76 | 452 | 36 |
| 528 | 1.44 | 507.4 | 34 |
| 529 | 1.00 | 565 | 36 |
| 530 | 0.61 | 465 | 36 |
| 532 | 1.30 | 565 | 35 |
| 533 | 0.68 | 493 | 36 |
| 534 | 1.51 | 466.3 | 16 |

### Analytical SFC

### General Procedure for SFC Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (NIS), It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 1c
Analytical SFC Methods (Flow expressed in mL/min; column temperature (T) in
° C.; Run time in minutes, Backpressure (BPR) in bars or pound-force per square
inch (psi). “ACN” means acetonitrile; “MeOH” means methanol; “EtOH” means
ethanol; “iPrNH₂” means isopropylamine. All other abbreviations used in the table
below are as defined before

| | | | | Flow- | |
|---|---|---|---|---|---|
| | | | | (ml/mn) | Run time |
| | | | | — | (min) |
| Method | | | | Column T | — |
| code | Column | Mobile phase | Gradient | (° C.) | BPR (bar) |
| 1 | Daicel | A: supercritical | 10%-50% B in 6 | 2.5 | 9.5 |
| | Chiralpak ® | CO₂ | min, hold 3.5 min | — | — |
| | AD3 | B: iPrOH + 0.2% | | 40 | 130 |
| | column (3.0 | iPrNH₂ | | | |
| | μm, 150 × 4.6 | | | | |
| | mm) | | | | |
| 2 | Daicel | A: supercritical | 10%-50% B in 6 | 2.5 | 9.5 |
| | Chiralpak ® | CO₂ | min, hold 3.5 min | — | — |
| | IG3 | B: EtOH + 0.2% | | 40 | 130 |
| | column (3.0 | iPrNH₂ | | | |
| | μm, 150 × 4.6 | | | | |
| | mm) | | | | |

### NMR:

### NMR-Methods

Some NMR experiments were carried out using a Bruker Avance III 400 spectrometer at ambient temperature (298.6 K), using internal deuterium lock and equipped with BBO 400 MHz S1 5 mm probe head with z gradients and operating at 400 MHz for the proton and 100 MHz for carbon. Chemical shifts (d) are reported in parts per million (ppm). J values are expressed in Hz.

Some NMR experiments were carried out using a Varian 400-MR spectrometer at ambient temperature (298.6 K), using internal deuterium lock and equipped with Varian 400 4NUC PFG probe head with z gradients and operating at 400 MHz for the proton and 100 MHz for carbon. Chemical shifts (d) are reported in parts per million (ppm). J values are expressed in Hz.

Some NMR experiments were carried out using a Varian 400-VNMRS spectrometer at ambient temperature (298.6 K), using internal deuterium lock and equipped with Varian 400 ASW PFG probe head with z gradients and operating at 400 MHz for the proton and 100 MHz for carbon. Chemical shifts (d) are reported in parts per million (ppm). J values are expressed in Hz.

| Compound | |
|---|---|
| number | NMR data |
| Compound 8 | ¹H NMR (400 MHZ, DMSO-d6) 8.43-8.33 (m, 1H), 7.92 (d, J = 7.2 Hz, |
| | 1H), 7.65-7.62 (m, 1H), 7.50-7.42 (m, 2H), 7.27-7.22 (m, 1H), 6.54 (s, |
| | 3.0H), 4.43-4.36 (m, 0.54H), 3.52-3.46 (m, 0.55H), 3.37-3.35 (m, 4H), |
| | 2.93-2.89 (m, 2H), 2.63-2.58 (m, 6H), 2.36-2.34 (m, 8H), 1.96 (s, 3H), |
| | 1.84 (s, 6H), 1.48-1.36 (m, 5H), 0.96-0.83 (m, 9H), 0.46-0.16 (m, 3H). |
| Compound 8a | ¹H NMR (400 MHz, METHANOL-d4) = 8.46 (s, 1H), 8.41 (d, J = 11.6 |
| | Hz, 1H), 7.98 (d, J = 4.8 Hz, 1H), 7.70-7.64 (m, 1H), 7.55-7.43 (m, |
| | 2H), 7.38 (dd, J = 2.4, 8.0 Hz, 1H), 3.84-3.46 (m, 8H), 3.26-3.07 (m, |
| | 4H), 2.89-2.71 (m, 4H), 2.69-2.47 (m, 9H), 2.31 (s, 2H), 2.10 (s, 3H), |
| | 1.96-1.54 (m, 5H), 1.31 (d, J = 18.0 Hz, 1H), 1.13-0.83 (m, 9H), 0.69- |
| | 0.20 (m, 2H) |
| Compound 9a | ¹H NMR (400 MHZ, METHANOL-d4) = 8.43-8.34 (m, 1H), 7.93 (d, |
| | J = 8.0 Hz, 1H), 7.66-7.60 (m, 1H), 7.47-7.40 (m, 1H), 7.37-7.31 (m, |
| | 2H), 3.54 (d, J = 18.0 Hz, 4H), 3.10-2.91 (m, 3H), 2.81-2.67 (m, 6H), |
| | 2.51-2.33 (m, 8H), 2.09 (s, 3H), 2.03 (d, J = 5.2 Hz, 2H), 1.75-1.40 (m, |
| | 4H), 1.31 (d, J = 18.0 Hz, 5H), 1.05-0.88 (m, 9H), 0.53 (s, 1H), 0.22 (dd, |
| | J = 6.4, 12.0 Hz, 2H) |
| Compound 32a | ¹H NMR (400 MHZ, DMSO-d6) 8.42-8.34 (m, 1H), 7.93-7.92 (m, 1H), |
| | 7.65-7.62 (m, 1H), 7.48-7.41 (m, 2H), 7.18-7.15 (m, 1H), 4.42-4.38 (m, |
| | 0.54H), 3.50-3.45 (m, 0.55H), 3.37 (s, br, 5H), 3.07-3.05 (m, 2H), 2.89- |
| | 2.81 (m, 2H), 2.60-2.56 (m, 5H), 2.33-2.30 (m, 2H), 2.18-2,11 (m, 4H), |
| | 1.96 (s, 3H), 1.87 (s, 10H), 1.67-1.60 (m, 2H), 1.52-1.47 (m, 1H), 0.94- |
| | 0.81 (m, 7H), 0.48-0.18 (m, 1H). |
| Compound 43 | ¹H NMR (400 MHZ, DMSO-d6) d 8.43-8.34 (m, 1H), 7.94-7.92 (m, |
| | 1H), 7.65-7.62 (m, 1H), 7.50-7.42 (m, 2H), 7.28-7.22 (m, 1H), 4.41- |
| | 4.39 (m, 0.5H), 3.80 (dd, J = 7.2 Hz; 3.6 Hz, 2H), 3.51-3.48 (m, 1H), |
| | 3.31-3.23 (m, 2.5H), 2.93-2.86 (m, 2H), 2.68-2.66 (m, 1H), 2.61- |
| | 2.58 (m, 5H), 2.55-2.51 (m, 0.5H), 2.48-2.46 (m, 0.5H), 2.44-2.37 |
| | (m, 1.5H), 2.34 (s, 1.5H), 2.33-2.18 (m, 3H), 1.96-1.95 (m,1H), 1.68- |
| | 1.60 (m, 3H), 1.49-1.45 (m, 1H), 1.16-1.06 (m, 2H), 0.96-0.94 (m, |
| | 2.5H), 0.48-0.47 (m, 1H), 0.20-0.17 (m, 1.5H). |
| Compound 51 | ¹H NMR (400 MHZ, DMSO-d6): δ 8.43-8.34 (m, 1H), 7.94-7.92 (m, |
| | 1H), 7.67-7.62 (m, 1H), 7.50-7.42 (m, 2H), 7.28-7.22 (m, 1H), 4.42-4.39 |
| | (m, 0.43H), 4.34-4.31 (m, 1H), 3.78-3.74 (m, 1H), 3.51-3.46 (m, 0.53H), |
| | 3.00-2.84 (m, 3H), 2.71-2.55 (m, 7H), 2.48-2.34 (m, 4H), 2.28-2.14 (m, |
| | 3H), 2.00-1.90 (m, 4H), 1.75-1.65 (m, 3H), 1.50-1.44 (m, 1H), 1.06-0.84 |
| | (m, 5H), 0.48-0.17 (m, 3H). |
| Compound 51a | ¹H NMR (400 MHZ, DMSO-d₆, 27° C.) δ ppm 0.18-0.53 (m, 3 H), 0.89- |
| | 1.29 (m, 5 H), 1.68-1.89 (m, 3 H), 1.98 (d, J = 1.3 Hz, 4 H), 2.07-2.29 |
| | (m, 1 H), 2.37 (s, 1 H), 2.57 (s, 2 H), 2.63 (s, 3 H), 2.94-3.15 (m, 6 H), |
| | 3.48-3.54 (m, 0.5 H), 3.56-3.75 (m, 2 H), 3.76-3.86 (m, 1 H), 4.35 |
| | (br d, J = 14.3 Hz, 2 H), 4.38-4.42 (m, 0.5 H), 7.32-7.42 (m, 1 H), 7.44- |
| | 7.54 (m, 2 H), 7.62-7.71 (m, 1 H), 7.97 (d, J = 5.5 Hz, 1 H), 8.34-8.47 |
| | (m, 1 H), 10.01-10.48 (m, 1 H). |
| Compound 59 | ¹H NMR (400 MHZ, DMSO-d6) δ = 8.56-8.25 (m, 1H), 8.04-7.82 (m, |
| | 1H), 7.70-7.58 (m, 1H), 7.56-7.38 (m, 2H), 7.33-7.17 (m, 1H), 4.49- |
| | 4.35 (m, 0.5H), 4.35-4.24 (m, 1H), 4.10-3.93 (m, 2H), 3.82-3.66 (m, |
| | 1H), 3.55-3.37 (m, 0.5H), 3.27-3.21 (m, 3H), 2.99-2.79 (m, 3H), |
| | 2.73-2.55 (m, 7H), 2.43-2.30 (m, 3H), 2.30-2.09 (m, 4H), 2.04-1.84 |
| | (m, 1H), 1.81-1.59 (m, 3H), 1.54-1.36 (m, 1H), 1.12-0.81 (m, 5H), |
| | 0.63-0.10 (m, 3H). |
| Compound 60 | ¹H NMR (400 MHZ, DMSO-d6) δ = 8.48-8.27 (m, 1H), 7.98-7.90 (m, |
| | 1H), 7.72-7.60 (m, 1H), 7.53-7.40 (m, 2H), 7.31-7.19 (m, 1H), 4.48- |
| | 4.33 (m, 0.5H), 4.33-4.23 (m, 1H), 4.08-3.92 (m, 2H), 3.66-3.57 (m, |
| | 1H), 3.55-3.42 (m, 0.5H), 3.08-2.79 (m, 3H), 2.74-2.55 (m, 7H), |
| | 2.43-2.11 (m, 6H), 2.06-1.82 (m, 1H), 1.80-1.61 (m, 3H), 1.56-1.37 |
| | (m, 1H), 1.16-0.82 (m, 5H), 0.62-0.09 (m, 3H). |
| Compound 117a | ¹H NMR (400 MHZ, METHANOL-d4) 8.44-8.32 (m, 1H), 7.92 (d, J = |
| | 7.6 Hz, 1H), 7.67-7.59 (m, 1H), 7.47-7.39 (m, 1H), 7.38-7.30 (m, |
| | 2H), 4.52-4.46 (m, 0.5H), 4.22-4.07 (m, 2H), 3.92-3.84 (m, 1H), |
| | 3.63-3.54 (m, 0.5H), 3.39 (s, 3H), 3.11-2.80 (m, 4H), 2.79-2.48 (m, |
| | 9H), 2.46-1.85 (m, 6H), 1.84-1.74 (m, 1H), 1.72-1.51 (m, 2H), 1.30- |
| | 1.12 (m, 2H), 1.10-0.87 (m, 6H), 0.59-0.18 (m, 3H) |
| Compound 125 | ¹H NMR (400 MHZ, DMSO-d6) δ ppm −0.04 (br d, J = 2.1 Hz, 1 H), 0.13- |
| | 0.22 (m, 2 H), 0.36-0.60 (m, 1 H), 0.85-0.88 (m, 1 H), 0.90-0.99 |
| | (m, 3 H), 1.24 (br s, 1 H), 1.29 (s, 6 H), 1.41-1.53 (m, 1 H), 1.64 (br d, |
| | J = 1.7 Hz, 1 H), 1.67-1.75 (m, 2 H), 1.87-2.02 (m, 1 H), 2.13-2.28 |
| | (m, 3 H), 2.32-2.42 (m, 3 H), 2.56-2.63 (m, 5 H), 2.64-2.74 (m, 1 H), |
| | 2.77-3.01 (m, 3 H), 3.14-3.27 (m, 1 H), 3.38-3.58 (m, 1 H), 4.33- |
| | 4.48 (m, 1 H), 5.30 (s, 1 H), 7.23 (d, J = 8.2 Hz, 1 H), 7.28 (s, 1 H), 7.42- |
| | 7.51 (m, 2 H), 7.61-7.68 (m, 1 H), 7.93 (d, J = 7.9 Hz, 1 H), 8.30-8.47 |
| | (m, 1 H), 8.34 (s, 1 H), 8.43 (d, J = 2.6 Hz, 1 H) |
| Compound 169a | ¹H NMR (400 MHZ, DMSO-d6, 27° C.) d ppm 0.12-0.25 (m, 2 H), 0.36- |
| | 0.63 (m, 1 H), 0.81-1.12 (m, 5 H), 1.33-1.55 (m, 1 H), 1.59-2.06 |
| | (m, 4 H), 2.08-2.27 (m, 4 H), 2.34 (s, 5 H), 2.56-2.65 (m, 5 H), 2.79- |
| | 3.16 (m, 4 H), 3.42 (s, 1 H), 4.32-4.48 (m, 1 H), 7.18-7.31 (m, 1 H), |
| | 7.32-7.54 (m, 3 H), 7.57-7.72 (m, 1 H), 7.86-7.99 (m, 1 H), 8.27- |
| | 8.51 (m, 1 H) |
| Compound 228 | ¹H NMR (400 MHZ, DMSO-d6, 100° C.) d ppm 0.25-0.89 (m, 6 H), |
| | 0.95 (s, 2 H), 1.04 (s, 3 H), 1.39-1.52 (m, 1 H), 1.61-1.72 (m, 2 H), |
| | 1.78-1.85 (m, 1 H), 1.87-1.95 (m, 2 H), 1.97 (s, 3 H), 2.09 (dd, |
| | J = 12.1, 4.5 Hz, 1 H), 2.16 (quin, J = 6.2 Hz, 1 H), 2.25 (q, J = 8.4 Hz, 1 |
| | H), 2.41 (br s, 1.5 H rotamer), 2.51-2.52 (m, 1 H), 2.60 (s, 1.5 |
| | Hrotamer), 2.62-2.65 (m, 3 H), 2.72 (br dd, J = 14.9, 9.3 Hz, 1 H), 3.02- |
| | 3.11 (m, 2 H), 3.51-3.62 (m, 0.5 H rotamer), 3.64-4.33 (m, 4 H), 4.35- |
| | 4.44 (m, 0.5 H rotamer), 7.25 (s, 1 H), 7.35 (dd, J = 8.5, 3.0 Hz, 1 H), |
| | 7.43 (td, J = 8.5, 3.0 Hz, 1 H), 7.58 (dd, J = 8.8, 5.0 Hz, 1 H), 7.94 (br s, 1 |
| | H), 8.29-8.44 (m, 1 H) |
| Compound 365b | ¹H NMR (400 MHZ, DMSO-d6, 27° C.) δ ppm 0.28 (br d, J = 6.2 Hz, 1 H), |
| | 0.39 (br d, J = 6.4 Hz, 1 H), 0.68 (d, J = 6.3 Hz, 1 H), 0.74-0.83 (m, 2 H), |
| | 0.83-0.91 (m, 2 H), 0.91-1.00 (m, 2 H), 1.04-1.16 (m, 2 H), 1.42- |
| | 1.52 (m, 1 H), 1.56-1.67 (m, 3 H), 1.92-2.02 (m, 1 H), 2.17-2.29 (m, |
| | 3 H), 2.32-2.48 (m, 2 H), 2.52-2.65 (m, 5 H), 2.81-2.92 (m, 3 H), |
| | 2.96 (br dd, J = 13.6, 5.7 Hz, 1 H), 3.10 (br dd, J = 13.6, 6.2 Hz, 1 H), 3.18- |
| | 3.30 (m, 3 H), 3.39-3.62 (m, 2 H), 3.67-3.89 (m, 3 H), 4.57-4.77 |
| | (m, 1 H), 7.24-7.29 (m, 1 H), 7.37-7.56 (m, 2 H), 7.56-7.64 (m, 1 H), |
| | 7.90-7.94 (m, 1 H), 8.26-8.37 (m, 1 H) |

### DSC

For a number of compounds, melting points (MP) were determined with a TA Instrument (Discovery DSC 250 or a DSC 2500). Melting points were measured with a temperature gradient of 10° C./minute. Maximum temperature was 300° C. Values are melting peak onset values.

### XPRD

Compound 51 as a Crystalline Free Base Form Compound 51 as a crystalline free base Form may be characterized by an X-ray powder diffraction pattern.

X-ray powder diffraction (XRPD) analysis was carried out on a PANalytical Empyrean diffractometer. The compound was loaded onto a zero-background silicon wafer sample holder by gently pressing the powder sample onto the flat surface.

Samples were run on XRPD using the method below:
Radiation: Cu K-Alpha (λ=1.5418 Å)
Tube voltage/current: 45 kV/40 mA
Divergence slit: ⅛°
Geometry: Bragg-Brentano
Scan mode: Continuous Scan
Scan Range: 3-40° 2θ
Step size: 0.013° 2θ
Scan speed: 20.4 s/step
Rotation: On
Detector: PIXcel^{1D}

One skilled in the art will recognize that diffraction patterns and peak positions are typically substantially independent of the diffractometer used and whether a specific calibration method is utilized. Typically, the peak positions may differ by about ±0.2° 2θ, or less. The intensities (and relative intensities) of each specific diffraction peak may also vary as a function of various factors, including but not limited to particle size, orientation, sample purity, etc.

The X-ray powder diffraction pattern comprises peaks at 9.3, 12.6, 15.7, 21.9 and 22.5° 2θ 0.2° 2θ. The X-ray powder diffraction pattern may further comprise at least one peak selected from 8.1, 11.6, 13.2, 16.8, 18.5, 18.7, 19.2, 19.9, 20.5° 2θ 0.2° 2θ.

Compound 51 as a crystalline free base Form may further be characterized by an X-ray powder diffraction pattern having four, five, six, seven, eight, nine or more peaks selected from those peaks identified in Table 2a.

Compound 51 as a crystalline free base Form may further be characterized by an X-ray powder diffraction pattern comprising those peaks identified in Table 2a, wherein the relative intensity of the peaks is greater than about 2%, preferably greater than about 5%, more preferably greater than about 10%, more preferably greater than about 15%. However, a skilled person will realize that the relative intensity of the peaks may vary between different samples and different measurements on the same sample.

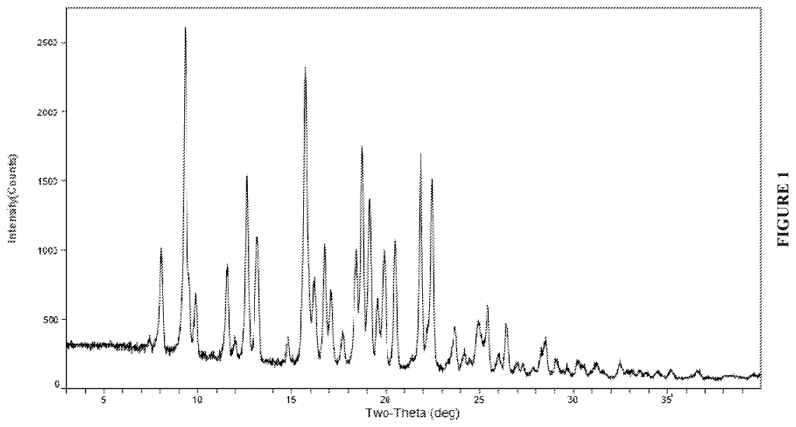

Compound 51 as a crystalline free base Form may further be characterized by an X-ray powder diffraction pattern substantially as depicted in FIG. 1.

Table 2a provides peak listing and relative intensity for the XPRD of Compound 51 as a crystalline free base Form:

| | Pos. | Rel. Int. | | Pos. | Rel. Int. |
|---|---|---|---|---|---|
| No. | (° 2θ) | (%) | No. | (° 2θ) | (%) |
| 1 | 7.411 | 4.6 | 16 | 18.744 | 63.6 |
| 2 | 8.056 | 31.4 | 17 | 19.15 | 48.8 |
| 3 | 9.304 | 100 | 18 | 19.57 | 16.4 |
| 4 | 9.893 | 18.2 | 19 | 19.912 | 32.2 |
| 5 | 11.574 | 28.1 | 20 | 20.503 | 38.2 |
| 6 | 11.969 | 6.4 | 21 | 21.881 | 65.4 |
| 7 | 12.598 | 55.9 | 22 | 22.485 | 57.1 |
| 8 | 13.163 | 37.6 | 23 | 23.693 | 13 |
| 9 | 14.804 | 7.9 | 24 | 24.205 | 5.6 |
| 10 | 15.723 | 89.6 | 25 | 24.915 | 15.2 |
| 11 | 16.195 | 24.4 | 26 | 25.401 | 19.6 |
| 12 | 16.762 | 34.5 | 27 | 26.068 | 5.3 |
| 13 | 17.076 | 21.2 | 28 | 26.400 | 14.5 |
| 14 | 17.694 | 9.5 | 29 | 28.276 | 8 |
| 15 | 18.454 | 33.2 | 30 | 28.499 | 11 |

### Compound 51a Crystalline HCl Salt Form (Mono HCl Trihydrate Salt)

Compound 51a (Crystalline HCl salt Form —mono HCl trihydrate salt) may be characterized by an X-ray powder diffraction pattern.

X-ray powder diffraction (XRPD) analysis was carried out on a PANalytical Empyrean diffractometer. The compound was loaded onto a zero-background silicon wafer sample holder by gently pressing the powder sample onto the flat surface.

Samples were run on XRPD using the method below:
Radiation: Cu K-Alpha (λ=1.5418 Å)
Tube voltage/current: 45 kV/40 mA
Divergence slit: ⅛°
Geometry: Bragg-Brentano
Scan mode: Continuous Scan
Scan Range: 3-40° 2θ
Step size: 0.013° 2θ
Scan speed: 20.4 s/step
Rotation: On
Detector: PIXcel^{1D}

One skilled in the art will recognize that diffraction patterns and peak positions are typically substantially independent of the diffractometer used and whether a specific calibration method is utilized. Typically, the peak positions may differ by about ±0.2° 2θ, or less. The intensities (and relative intensities) of each specific diffraction peak may also vary as a function of various factors, including but not limited to particle size, orientation, sample purity, etc.

The X-ray powder diffraction pattern comprises peaks at 5.2, 13.2, 14.1, 18.8 and 20.3° 2θ 0.2° 2θ. The X-ray powder diffraction pattern may further comprise at least one peak selected from 9.7, 10.0, 15.4, 15.8, 18.3, 21.3, 24.3° 2θ±0.2° 2θ.

Compound 51a may further be characterized by an X-ray powder diffraction pattern having four, five, six, seven, eight, nine or more peaks selected from those peaks identified in Table 2b.

Compound 51a may further be characterized by an X-ray powder diffraction pattern comprising those peaks identified in Table 2b, wherein the relative intensity of the peaks is greater than about 2%, preferably greater than about 5%, more preferably greater than about 10%, more preferably greater than about 15%. However, a skilled person will realize that the relative intensity of the peaks may vary between different samples and different measurements on the same sample.

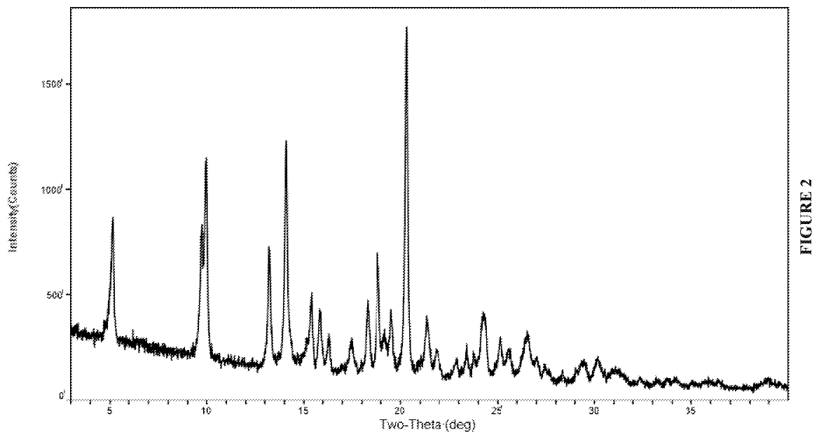

Compound 51a may further be characterized by an X-ray powder diffraction pattern substantially as depicted in FIG. 2.

Table 2b provides peak listing and relative intensity for the XPRD of Compound 51a.

| | Pos. | Rel. Int. | | Pos. | Rel. Int. |
|---|---|---|---|---|---|
| No. | (° 2θ) | (%) | No. | (° 2θ) | (%) |
| 1 | 5.151 | 36 | 13 | 19.505 | 16.6 |
| 2 | 9.749 | 37.2 | 14 | 20.305 | 100 |
| 3 | 9.984 | 58.9 | 15 | 21.331 | 16.6 |
| 4 | 13.217 | 34 | 16 | 21.855 | 6.8 |
| 5 | 14.095 | 64.4 | 17 | 22.905 | 4.5 |
| 6 | 15.393 | 20.4 | 18 | 23.419 | 8.2 |
| 7 | 15.842 | 16.4 | 19 | 24.310 | 17.4 |
| 8 | 16.315 | 10.4 | 20 | 25.136 | 10.6 |
| 9 | 17.471 | 10.1 | 21 | 25.595 | 7.1 |
| 10 | 18.296 | 19.4 | 22 | 26.529 | 12.9 |
| 11 | 18.810 | 34.3 | 23 | 29.496 | 4.5 |
| 12 | 19.19 | 10.8 | 24 | 30.179 | 6.1 |

### Dynamic Vapor Sorption (DVS)

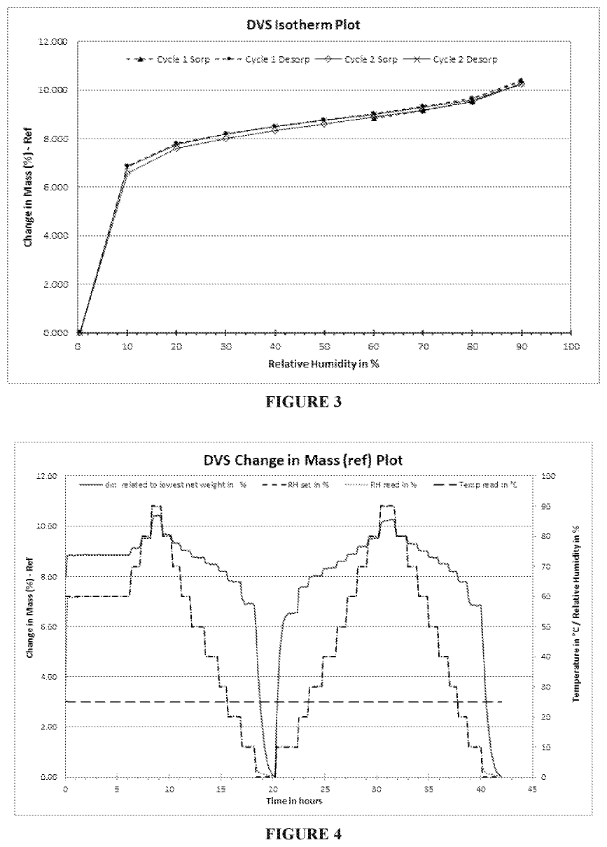

The moisture sorption analysis (DVS) was performed using a ProUmid GmbH & Co. KG Vsorp Enhanced dynamic vapor sorption apparatus. Results are shown in FIG. 3 and FIG. 4. The moisture profile was evaluated by monitoring vapor adsorption/desorption over the range of 0 to 90% relative humidity at 25° C. The sample weight equilibrium criteria were set at <0.01% change in 45 min with minimum and maximum time of acclimation at 50 min and 120 min, respectively. The moisture profile consisted of 2 cycles of vapor adsorption/desorption.

The DVS change in mass plot of crystalline HCl salt Form (Compound 51a) shows that the crystalline form is hygroscopic with the water content varying with relative humidity and dehydrates rapidly at below 10% RH (relative humidity) to complete dehydrated state at 0% RH. In the humidity range of 20-90% RH, the crystalline form adsorbs and desorbs moisture slowly and reversibly up to 2.5% by mass on average. Based on DVS, the crystalline HCl salt Form, at equilibrium, can contain around 3 equivalents of water (8.5-9.5% total moisture mass) at common ambient RH of 40% to 75%. The XRPD pattern of the fraction obtained after the DVS test was comparable to the starting material. No indication of a solid-state form change was observed.

### Pharmacological Part

### 1) Menin/MLL Homogenous Time-Resolved Fluorescence (HTRF) Assay

To an untreated, white 384-well microtiter plate was added 40 nL 200× test compound in DMSO and 4 μL 2× terbium chelate-labeled menin (vide infra for preparation) in assay buffer (40 mM Tris HCl, pH 7.5, 50 mM NaCl. 1 mM DTT (dithiothreitol) and 0.05% Pluronic F-127). After incubation of test compound and terbium chelate-labeled menin for 30 min at ambient temperature, 4 μL 2×FITC-MBM1 peptide (FITC-β-alanine-SARWRFPARPGT-NH₂) (“FITC” means fluorescein isothiocyanate) in assay buffer was added, the microtiter plate centrifuged at 1000 rpm for 1 min and the assay mixtures incubated for 15 min at ambient temperature. The relative amount of menin FITC-MBM1 complex present in an assay mixture is determined by measuring the homogenous time-resolved fluorescence (HTRF) of the terbium/FITC donor/acceptor fluorphore pair using an EnVision microplate reader (ex. 337 nm/terbium em. 490 nm/FITC em. 520 nm) at ambient temperature. The degree of fluorescence resonance energy transfer (the HTRF value) is expressed as the ratio of the fluorescence emission intensities of the FITC and terbium fluorophores (F^{em}520 nm/F^{em}490 nm). The final concentrations of reagents in the binding assay are 200 μM terbium chelate-labeled menin, 75 nM FITC-MBM1 peptide and 0.5% DMSO in assay buffer. Dose-response titrations of test compounds are conducted using an 11 point, four-fold serial dilution scheme, starting typically at 10 μM.

Compound potencies were determined by first calculating % inhibition at each compound concentration according to equation 1:

% inhibition=((HC−LC)−(HTRF^{compound}−LC))/(HC−LC))*100 (Eqn 1)

Where LC and HC are the HTRF values of the assay in the presence or absence of a saturating concentration of a compound that competes with FITC-MBM1 for binding to menin, and HTRF^{compound} is the measured HTRF value in the presence of the test compound. HC and LC HTRF values represent an average of at least 10 replicates per plate. For each test compound, % inhibition values were plotted vs. the logarithm of the test compound concentration, and the IC₅₀ value derived from fitting these data to equation 2:

% inhibition=Bottom+(Top−Bottom)/(1+10\hat{ }((log/C₅₀−log[cmpd])*h)) (Eqn 2)

Where Bottom and Top are the lower and upper asymptotes of the dose-response curve, respectively, IC₅₀ is the concentration of compound that yields 50% inhibition of signal and h is the Hill coefficient.

Preparation of Terbium cryptate labeling of Menin: Menin (a.a 1-610-6xhis tag, 2.3 mg/mL in 20 mM Hepes (2-[4-(2-Hydroxyethyl)-1-piperazinyl]ethane sulfonic acid), 80 mM NaCl, 5 mM DTT (Dithiothreitol), pH 7.5) was labeled with terbium cryptate as follows. 200 μg of Menin was buffer exchanged into 1×Hepes buffer. 6.67 μM Menin was incubated with 8-fold molar excess NHS (N-hydroxysuccinimide)-terbium cryptate for 40 minutes at room temperature. Half of the labeled protein was purified away from free label by running the reaction over a NAP5 column with elution buffer (0.1M Hepes, pH 7+0.1% BSA (bovine serum albumin)). The other half was eluted with 0.1 M phosphate buffered saline (PBS), pH7. 400 μl of eluent was collected for each, aliquoted and frozen at −80° C. The final concentration of terbium-labeled Menin protein was 115 μg/mL in Hepes buffer and 85 μg/mL in PBS buffer, respectively.

MENIN Protein Sequence (SEQ ID NO: 1):
MGLKAAQKTLFPLRSIDDVVRLFAAELGREEPDLVLLSLVLGFVEHFLAV
NRVIPTNVPELTFQPSPAPDPPGGLTYFPVADLSIIAALYARFTAQIRGA
VDLSLYPREGGVSSRELVKKVSDVIWNSLSRSYFKDRAHIQSLFSFITGT
KLDSSGVAFAVVGACQALGLRDVHLALSEDHAWVVFGPNGEQTAEVTWHG
KGNEDRRGQTVNAGVAERSWLYLKGSYMRCDRKMEVAFMVCAINPSIDLH
TDSLELLQLQQKLLWLLYDLGHLERYPMALGNLADLEELEPTPGRPDPLT
LYHKGIASAKTYYRDEHIYPYMYLAGYHCRNRNVREALQAWADTATVIQD
YNYCREDEEIYKEFFEVANDVIPNLLKEAASLLEAGEERPGEQSQGTQSQ
GSALQDPECFAHLLRFYDGICKWEEGSPTPVLHVGWATFLVQSLGRFEGQ
VRQKVRIVSREAEAAEAEEPWGEEAREGRRRGPRRESKPEEPPPPKKPAL
DKGLGTGQGAVSGPPRKPPGTVAGTARGPEGGSTAQVPAPAASPPPEGPV
LTFQSEKMKGMKELLVATKINSSAIKLQLTAQSQVQMKKQKVSTPSDYTL
SFLKRQRKGLHHHHHH

### 2a) Proliferation Assay

The anti-proliferative effect of menin/MLL protein/protein interaction inhibitor test compounds was assessed in human leukemia cell lines. The cell line MOLM14 harbors a MLL translocation and expresses the MLL fusion protein MLL-AF9, respectively, as well as the wildtype protein from the second allele. OCI-AML3 cells that carry the NPM1c gene mutation were also tested. MLL rearranged cell lines (e.g. MOLM14) and NPM1c mutated cell lines exhibit stem cell-like HOXA/MEIS1 gene expression signatures. KO-52 was used as a control cell line containing two MLL (KMT2A) wildtype alleles in order to exclude compounds that display general cytotoxic effects.

MOLM14 cells were cultured in RPMI-1640 (Sigma Aldrich) supplemented with 10% heat-inactivated fetal bovine serum (HyClone), 2 mM L-glutamine (Sigma Aldrich) and 50 μg/ml gentamycin (Gibco). KO-52 and OCI-AML3 cell lines were propagated in alpha-MEM (Sigma Aldrich) supplemented with 20% heat-inactivated fetal bovine serum (HyClone), 2 mM L-glutamine (Sigma Aldrich) and 50 μg/ml gentamycin (Gibco). Cells were kept at 0.3-2.5 million cells per ml during culturing and passage numbers did not exceed 20.

In order to assess the anti-proliferative effects, 200 MOLM14 cells, 200 OCI-AML3 cells or 300 KO-52 cells were seeded in 200 μl media per well in 96-well round bottom, ultra-low attachment plates (Costar, catalogue number 7007). Cell seeding numbers were chosen based on growth curves to ensure linear growth throughout the experiment. Test compounds were added at different concentrations and the DMSO content was normalized to 0.3%. Cells were incubated for 8 days at 37° C. and 5% CO₂. Spheroid like growth was measured in real-time by live-cell imaging (IncuCyteZOOM, Essenbio, 4×objective) acquiring images at day 8. Confluence (%) as a measure of spheroid size was determined using an integrated analysis tool.

In order to determine the effect of the test compounds over time, the confluence in each well as a measure of spheroid size, was calculated. Confluence of the highest dose of a reference compound was used as baseline for the LC (Low control) and the confluence of DMSO treated cells was used as 0% cytotoxicity (High Control, HC).

Absolute IC₅₀ values were calculated as percent change in confluence as follows:

LC=Low Control: cells treated with e.g. 1 μM of the cytotoxic agent staurosporin, or e.g. cells treated with a high concentration of an alternative reference compound

HC=High Control: Mean confluence (%) (DMSO treated cells)

% Effect=100−(100*(Sample−LC)/(HC−LC))

GraphPad Prism (version 7.00) was used to calculate the IC₅₀. Dose-response equation was used for the plot of % Effect vs Log10 compound concentration with a variable slope and fixing the maximum to 100% and the minimum to 0%.

2b) MEIS1 mRNA Expression Assay

MEIS1 mRNA expression upon treatment of compound was examined by Quantigene Singleplex assay (Thermo Fisher Scientific). This technology allows for direct quantification of mRNA targets using probes hybridizing to defined target sequences of interest and the signal is detected using a Multimode plate reader Envision (PerkinElmer). The MOLM14 cell line was used for this experiment. Cells were plated in 96-well plates at 3,750 cells/well in the presence of increasing concentrations of compounds. After incubation of 48 hours with compounds, cells were lysed in lysis buffer and incubated for 45 minutes at 55° C. Cell lysates were mixed with human MEIS specific capture probe or human RPL28 (Ribosomal Protein L28) specific probe as a normalization control, as well as blocking probes. Cell lysates were then transferred to the custom assay hybridization plate (Thermo Fisher Scientific) and incubated for 18 to 22 hours at 55° C. Subsequently, plates were washed to remove unbound materials followed by sequential addition of preamplifiers, amplifiers, and label probe. Signals (═gene counts) were measured with a Multimode plate reader Envision. IC_{50S} were calculated by dose-response modelling using appropriate software. For all non-housekeeper genes response equal counts corrected for background and relative expression. For each sample, each test gene signal (background subtracted) was divided by the normalization gene signal (RPL28: background subtracted). Fold changes were calculated by dividing the normalized values for the treated samples by the normalized values for the DMSO treated sample. Fold changes of each target gene were used for the calculation of IC_{50S}.

TABLE 3
Biological data - HTRF assay, proliferation assay, and MEIS1
mRNA expressionassay

| | | | | spheroid | |
|---|---|---|---|---|---|
| | HTRF-30 min | MEIS1 | spheroid assay_ | assay_OCI- | spheroid assay_KO- |
| Compound | incubation IC₅₀ | IC₅₀ | MOLM14 IC₅₀ | AML3 | 52 IC₅₀ |
| Number | (μM) | (μM) | (μM) | IC₅₀ (μM) | (μM) |
| 1 | 0.000033 | 0.004 | 0.002 | | 7.5 |
| 2 | 0.000049 | 0.003 | 0.001 | | >15 |
| 2a | 0.000024 | 0.036 | | | |
| 3 | 0.000026 | 0.010 | 0.004 | | 1.1 |
| 4 | 0.000016 | 0.011 | 0.005 | | 3.0 |
| 4a | 0.000301 | 0.247 | 0.132 | | 7.8 |
| 4b | 0.000094 | 0.006 | 0.003 | | 0.5 |
| 6 | 0.000288 | 0.049 | 0.034 | | 1.5 |
| 7 | 0.000305 | 0.158 | | | |
| 8 | 0.000365 | 0.023 | 0.011 | | 2.4 |
| 8a | 0.000024 | 0.013 | | | |
| 8b | ~0.0073 | 0.044 | | | |
| 9 | 0.000138 | 0.023 | 0.013 | | 5.6 |
| 9a | 0.000016 | 0.006 | 0.002 | | 0.6 |
| 9b | 0.000975 | 0.042 | | | |
| 10 | 0.000021 | 0.017 | | | |
| 11 | 0.000034 | 0.006 | | | |
| 12 | 0.000018 | 0.005 | | | |
| 13 | 0.000124 | 0.006 | | | |
| 14 | 0.000054 | <0.0036 | | | |
| 15 | 0.000040 | <0.0008 | | | |
| 16 | 0.000051 | 0.070 | | | |
| 17 | 0.000031 | 0.048 | | | |
| 18a | 0.000019 | 0.005 | | | |
| 18b | 0.000017 | 0.110 | | | |
| 19a | 0.000032 | <0.0033 | 0.003 | | >15 |
| 19b | 0.000065 | 0.128 | | | |
| 20 | ~0.00019 | <0.0017 | | | |
| 21 | 0.000052 | <0.0023 | <0.0018 | | 2.0 |
| 22 | 0.000024 | ~0.011 | | | |
| 23 | 0.000070 | 0.007 | | | |
| 24 | 0.000057 | 0.071 | 0.013 | | 8.5 |
| 25 | 0.000087 | 0.091 | | | |
| 26a | 0.000036 | 0.007 | | | |
| 26b | ~0.000036 | 0.029 | | | |
| 27a | 0.000012 | <0.0041 | | | |
| 27b | 0.000038 | 0.019 | | | |
| 27c | 0.000038 | 0.040 | | | |
| 27d | 0.000013 | 0.080 | | | |
| 28 | 0.000055 | 0.107 | | | |
| 28b | 0.000042 | 0.162 | | | |
| 30 | ~0.00049 | >1 | | | |
| 31 | 0.000052 | ~0.47 | | | |
| 32a | 0.000062 | 0.022 | 0.005 | | 5.5 |
| 32b | 0.000472 | >1 | | | |
| 33a | 0.000070 | 0.011 | 0.010 | | 3.0 |
| 33b | 0.000030 | 0.023 | | | |
| 33c | 0.000072 | ~0.42 | | | |
| 34a | 0.000019 | 0.016 | | | |
| 34b | 0.000092 | 0.025 | | | |
| 35a | 0.000059 | 0.021 | | | 1.5 |
| 35b | 0.000077 | 0.047 | | | |
| 36a | 0.000030 | 0.016 | | | 0.6 |
| 36b | 0.000052 | 0.019 | 0.003 | | 0.5 |
| 37a | 0.000126 | 0.027 | 0.008 | | 0.8 |
| 37b | 0.000237 | 0.028 | | | |
| 38a | 0.000036 | 0.006 | <0.0018 | | 0.8 |
| 38b | 0.000013 | | 0.002 | | 1.2 |
| 39a | 0.000034 | 0.005 | 0.002 | | >15 |
| 39b | 0.000052 | 0.035 | | | |
| 40a | 0.000841 | ~0.54 | | | |
| 40b | 0.000182 | ~0.68 | | | |
| 41 | 0.000202 | 0.015 | 0.014 | | 1.4 |
| 42 | 0.000030 | 0.045 | 0.031 | | 2.8 |
| 43 | 0.000027 | 0.024 | 0.014 | 0.023 | 3.3 |
| 44 | 0.000060 | 0.043 | 0.071 | | 4.7 |
| 45 | 0.000054 | 0.056 | 0.053 | | 6.2 |
| 46 | 0.000047 | 0.092 | | | |
| 47 | 0.000042 | 0.055 | | | |
| 48 | 0.000060 | 0.020 | | | |
| 49 | 0.000048 | 0.017 | 0.003 | | 3.0 |
| 49a | 0.000227 | 0.221 | 0.113 | | |
| 49b | 0.000060 | 0.009 | 0.003 | 0.007 | 4.1 |
| 50 | 0.000079 | 0.089 | 0.039 | | 2.3 |
| 51 | 0.000042 | 0.011 | 0.008 | 0.024 | 1.9 |
| 51a | 0.000024 | 0.011 | 0.013 | 0.070 | 1.2 |
| 58 | 0.000135 | 0.183 | 0.176 | | 10.4 |
| 59 | 0.000054 | 0.009 | 0.011 | 0.012 | 2.1 |
| 60 | <0.0000095 | 0.007 | 0.013 | 0.013 | 2.7 |
| 61 | 0.000132 | 0.023 | 0.010 | | 7.9 |
| 62 | 0.000085 | 0.014 | | | |
| 63 | 0.000176 | 0.083 | 0.053 | 0.121 | 5.9 |
| 64 | 0.000284 | ~0.039 | | | |
| 65 | 0.000079 | 0.045 | | | |
| 66 | 0.000037 | 0.046 | 0.018 | | 7.7 |
| 67 | ~0.000043 | ~0.068 | 0.048 | | 12.0 |
| 68 | 0.000043 | 0.032 | | | |
| 69 | 0.000049 | 0.017 | 0.005 | 0.021 | 11.0 |
| 70 | 0.000114 | 0.048 | 0.040 | 0.068 | 8.5 |
| 71 | 0.000202 | 0.048 | 0.083 | | >15 |
| 72 | 0.000173 | 0.182 | | | |
| 73 | 0.000057 | 0.101 | | | |
| 74 | 0.000034 | 0.012 | 0.004 | | 5.2 |
| 75 | 0.000059 | 0.119 | 0.009 | | 2.0 |
| 76 | 0.000304 | | | | |
| 77 | 0.000191 | 0.136 | 0.090 | | 5.8 |
| 77a | 0.000105 | 0.206 | 0.117 | | |
| 77b | 0.000070 | 0.132 | 0.081 | 0.128 | 1.9 |
| 77c | 0.000546 | 0.669 | | | |
| 77d | 0.000208 | ~0.26 | 0.197 | | >15 |
| 78 | 0.000299 | 0.327 | 0.111 | | 8.4 |
| 78a | 0.001217 | ~0.69 | | | |
| 78b | 0.000552 | 0.361 | | | |
| 78c | 0.000382 | 0.416 | 0.200 | | |
| 78d | 0.000875 | 0.538 | | | |
| 79 | 0.000168 | | 0.092 | | 9.4 |
| 80 | 0.000116 | | 0.099 | | 7.2 |
| 81 | 0.000425 | 0.077 | 0.058 | 0.100 | 4.0 |
| 82 | ~0.000035 | 0.011 | | | |
| 83 | 0.001300 | 2.220 | | | |
| 84 | 0.000075 | 0.131 | 0.063 | 0.120 | 4.3 |
| 85 | 0.000113 | 0.022 | 0.012 | 0.022 | 2.2 |
| 86 | 0.000042 | 0.021 | 0.010 | 0.023 | 1.3 |
| 87 | 0.000042 | 0.027 | | | |
| 88 | 0.000093 | 0.140 | 0.096 | | |
| 89 | 0.000183 | ~0.895 | | | |
| 90a | 0.000391 | ~0.127 | | | |
| 90b | 0.000263 | 0.103 | | | |
| 91 | 0.000155 | 0.094 | | | |
| 92 | 0.000219 | 0.052 | | | |
| 93 | 0.000418 | ~0.360 | | | |
| 94 | 0.000079 | 0.309 | | | |
| 95 | 0.000601 | ~0.914 | | | |
| 96 | 0.004571 | 3.691 | | | |
| 97 | 0.001971 | 1.607 | | | |
| 98 | 0.000747 | ~0.535 | | | |
| 99 | 0.000217 | ~0.792 | | | |
| 100 | 0.000165 | 0.434 | | | |
| 101 | 0.000041 | 0.007 | 0.041 | 0.008 | 3.1 |
| 102 | 0.003025 | >1 | | | |
| 103 | 0.003272 | ~5.12 | | | |
| 104 | 0.000147 | 0.254 | 0.275 | | >15 |
| 105 | 0.000728 | >1 | | | |
| 106 | 0.000039 | 0.104 | | | |
| 107 | 0.000188 | 0.226 | | | |
| 108 | 0.000161 | 0.153 | | | |
| 109 | 0.000304 | 0.053 | | | |
| 110 | 0.001802 | ~1.07 | | | |
| 111 | 0.000745 | ~0.707 | | | |
| 112 | 0.000555 | 0.345 | | | |
| 113 | 0.000642 | ~0.493 | | | |
| 114 | 0.000173 | 0.487 | | | |
| 115 | 0.000021 | 0.011 | | | |
| 116a | 0.000075 | 0.005 | 0.008 | 0.009 | 3.8 |
| 116b | 0.000329 | 0.377 | | | |
| 117a | 0.000088 | 0.007 | 0.005 | 0.007 | 1.6 |
| 117b | 0.000270 | 0.411 | 0.300 | | |
| 118 | 0.000139 | 0.117 | | | |
| 119a | 0.000906 | >1 | | | |
| 119b | 0.000569 | >1 | | | |
| 120 | 0.000054 | 0.149 | 0.539 | | |
| 121 | 0.000049 | 0.013 | | | |
| 122 | 0.000050 | 0.018 | 0.012 | | |
| 123 | 0.000052 | 0.031 | 0.016 | | |
| 124 | 0.000156 | 0.114 | | | |
| 125 | 0.000027 | 0.016 | 0.011 | 0.012 | 7.0 |
| 126 | 0.000091 | 0.032 | 0.018 | | |
| 128 | 0.000078 | 0.238 | 0.146 | | 7.9 |
| 128a | 0.000151 | 0.258 | 0.676 | | 7.7 |
| 128b | 0.000045 | 0.065 | 0.040 | | |
| 128c | 0.001006 | ~0.4869 | | | |
| 128d | 0.000104 | 0.419 | 0.089 | | |
| 129 | 0.000291 | 0.277 | | | |
| 130 | 0.001202 | 0.314 | | | |
| 131 | 0.000132 | 0.129 | | | |
| 132 | 0.001211 | ~0.5733 | | | |
| 133 | 0.000051 | 0.042 | | | |
| 134a | 0.000032 | 0.086 | | | |
| 134b | 0.000988 | >1 | | | |
| 134c | 0.000073 | 0.119 | | | |
| 134d | 0.000425 | >1 | | | |
| 135 | ~0.000056 | 0.007 | | | |
| 136 | ~0.000094 | 0.071 | 0.028 | 0.054 | 1.4 |
| 137a | 0.000063 | 0.014 | 0.015 | 0.023 | 1.1 |
| 137b | ~0.00034 | 0.043 | | | |
| 138 | 0.000067 | 0.047 | | | |
| 138a | 0.000054 | 0.044 | 0.032 | 0.072 | 1.7 |
| 138b | 0.000109 | ~0.258 | 0.086 | 0.222 | 3.7 |
| 139 | 0.000119 | 0.444 | | | |
| 139a | 0.000122 | ~0.81 | | | |
| 139b | 0.000087 | 0.716 | | | |
| 140 | ~0.00017 | 0.049 | 0.015 | | |
| 141 | 0.000027 | 0.015 | | | |
| 142 | 0.000086 | 0.010 | | | |
| 143 | 0.000064 | 0.005 | | | |
| 144 | 0.000072 | 0.011 | 0.011 | | 6.5 |
| 145 | 0.001056 | ~0.7485 | | | |
| 146 | 0.001220 | >1 | | | |
| 147a | 0.000082 | 0.052 | 0.010 | | |
| 147b | 0.000073 | ~0.1048 | | | |
| 148 | 0.000086 | 0.238 | 0.088 | | |
| 149 | 0.000037 | 0.023 | | | |
| 150 | 0.000430 | >1 | 1.474 | | |
| 151 | 0.003773 | 1.351 | | | |
| 152 | 0.000053 | 0.197 | | | |
| 153 | 0.000077 | 0.077 | 0.035 | | 7.9 |
| 154 | 0.000140 | 0.244 | 0.091 | | |
| 155 | 0.000051 | 0.054 | 0.049 | | |
| 156 | 0.000042 | 0.187 | 0.042 | | |
| 157 | 0.000020 | 0.006 | 0.002 | | |
| 158 | 0.000045 | 0.024 | 0.006 | | |
| 159 | 0.000019 | 0.010 | 0.003 | | |
| 160 | 0.004021 | >1 | | | |
| 161 | 0.000167 | 0.256 | 0.053 | | |
| 162 | 0.000505 | >1 | 0.424 | | |
| 163 | 0.000192 | ~0.623 | 0.456 | | |
| 164a | 0.000209 | 0.249 | 0.154 | | |
| 164b | 0.000025 | 0.017 | 0.010 | 0.012 | 10.4 |
| 165a | 0.000173 | ~0.18 | | | |
| 165b | 0.000070 | 0.038 | 0.004 | | 1.9 |
| 166a | 0.000084 | 0.038 | 0.049 | | |
| 166b | 0.000226 | 0.256 | 0.120 | | |
| 167a | 0.000190 | >1 | 0.210 | | |
| 167b | 0.000118 | 0.117 | 0.099 | | |
| 168 | 0.000100 | 0.016 | 0.008 | | |
| 169 | 0.000061 | 0.071 | 0.016 | | 10.0 |
| 169a | 0.000061 | 0.015 | 0.017 | | |
| 169b | 0.000195 | ~0.22 | 0.087 | | |
| 170 | 0.000155 | 0.079 | 0.014 | | |
| 171 | 0.000581 | >1 | 0.337 | | |
| 172 | 0.000134 | 0.018 | 0.011 | | |
| 173 | 0.000056 | 0.032 | 0.005 | | |
| 174 | 0.000048 | 0.034 | 0.007 | | |
| 175 | 0.001332 | ~1.05 | 0.234 | | |
| 176 | 0.000200 | 0.306 | 0.046 | | |
| 177 | 0.000109 | 0.268 | 0.035 | | |
| 178 | 0.000055 | 0.014 | 0.012 | | 2.9 |
| 179 | 0.000405 | 0.259 | | | |
| 180a | 0.000143 | 0.117 | 0.039 | | 7.2 |
| 180b | 0.007291 | ~2.806 | | | |
| 181a | 0.000066 | 0.111 | 0.044 | | 4.1 |
| 181b | 0.004582 | 1.914 | | | |
| 182a | 0.000462 | 0.287 | | | |
| 182b | 0.003095 | 1.767 | | | |
| 183a | 0.000370 | 0.307 | | | |
| 183b | 0.002614 | ~2.04 | | | |
| 184 | 0.000060 | | 0.026 | | 7.4 |
| 184a | 0.000041 | 0.023 | 0.013 | 0.026 | >15 |
| 184b | 0.000065 | 0.115 | 0.052 | 0.138 | >15 |
| 185 | 0.000097 | 0.044 | 0.023 | | 11.0 |
| 186a | 0.000447 | 0.419 | | | |
| 186b | 0.000932 | 0.504 | | | |
| 187 | 0.001016 | 1.937 | | | |
| 189 | 0.000544 | 0.314 | 0.255 | | 14.4 |
| 190a | 0.000101 | 0.113 | 0.040 | | 10.0 |
| 190b | 0.000063 | 0.065 | 0.046 | | 4.9 |
| 191a | 0.000056 | 0.039 | 0.007 | | 4.6 |
| 191b | 0.000071 | 0.041 | 0.033 | | 7.0 |
| 192 | ~0.000268 | ~0.403 | | | |
| 193 | 0.000071 | 0.041 | | | |
| 194 | 0.000072 | 0.066 | | | |
| 195 | 0.000153 | 0.156 | | | |
| 196 | 0.000190 | 0.438 | | | |
| 197 | 0.000046 | 0.008 | | | |
| 198a | 0.000034 | 0.013 | 0.010 | 0.015 | |
| 198b | 0.000085 | 0.107 | | 0.156 | >15 |
| 199 | 0.000327 | 0.338 | 0.126 | | 11.8 |
| 200 | 0.000909 | ~0.864 | 0.679 | | >15 |
| 201 | 0.000063 | 0.047 | 0.031 | 0.035 | |
| 202 | 0.000081 | 0.025 | 0.007 | | 3.4 |
| 203 | 0.000069 | 0.043 | 0.012 | | 2.9 |
| 204 | 0.000091 | 0.079 | 0.016 | | 1.6 |
| 205 | 0.000106 | 0.019 | 0.019 | | 2.5 |
| 205a | 0.000049 | 0.021 | 0.023 | | 7.0 |
| 205b | 0.000035 | 0.031 | 0.025 | | 3.2 |
| 206a | 0.000045 | 0.024 | 0.013 | | 6.6 |
| 206b | 0.000387 | ~0.524 | 0.148 | | >15 |
| 207 | 0.000016 | 0.029 | 0.016 | | 14.3 |
| 208 | 0.000147 | 0.281 | 0.224 | | >15 |
| 209 | 0.000202 | >1 | 0.458 | | 12.8 |
| 210 | 0.000602 | >1 | | | |
| 211 | 0.000032 | 0.113 | 0.169 | | >15 |
| 212 | 0.000115 | 0.322 | 0.305 | | >15 |
| 213a | 0.000567 | 1.149 | | | |
| 213b | 0.001629 | ~2.30 | | | |
| 214 | | >1 | | | |
| 214a | 0.001293 | ~0.605 | | | |
| 214b | 0.011692 | ~7.28 | | | |
| 215 | 0.000291 | 0.299 | | | |
| 216 | 0.000187 | 0.100 | | | |
| 217 | 0.000039 | 0.021 | | | |
| 218 | 0.000094 | 0.048 | | | |
| 219 | 0.000039 | 0.032 | | | |
| 220 | 0.000932 | >1 | | | |
| 221 | 0.000311 | 0.109 | | | |
| 222 | 0.000103 | 0.259 | | | |
| 223 | 0.000239 | 0.279 | | | |
| 224 | 0.008656 | ~3.88 | | | |
| 224a | 0.030697 | >1 | | | |
| 224b | 0.002766 | >1 | | | |
| 225 | 0.000637 | 0.340 | | | |
| 226 | 0.000255 | 0.269 | | | |
| 227 | 0.000655 | 0.551 | | | |
| 228 | 0.000061 | 0.008 | 0.010 | 0.011 | 5.8 |
| 229 | 0.000235 | 0.093 | | | |
| 230a | 0.000053 | 0.077 | | | |
| 230b | 0.005446 | >1 | | | |
| 231a | 0.000311 | 0.052 | | | |
| 231b | 0.006942 | >1 | | | |
| 232a | 0.000624 | ~0.448 | | | |
| 232b | 0.004044 | >1 | | | |
| 233a | 0.000171 | 0.247 | | | |
| 233b | 0.002238 | >1 | | | |
| 234 | ~0.000068 | 0.018 | | | |
| 235 | 0.000097 | 0.014 | | | |
| 236 | 0.000086 | ~0.0989 | | | |
| 237 | 0.000149 | 0.052 | | | |
| 238 | 0.001935 | >1 | | | |
| 239 | 0.000305 | 0.138 | | | |
| 240a | 0.000407 | 0.291 | | | |
| 240b | 0.000151 | 0.038 | | | |
| 241 | 0.000084 | 0.173 | | | |
| 242 | 0.000811 | >1 | | | |
| 243 | 0.000097 | 0.037 | | | |
| 244 | 0.000101 | 0.051 | | | |
| 245 | 0.000185 | 0.065 | | | |
| 246 | 0.000057 | 0.016 | | | |
| 247 | 0.000179 | 0.116 | | | |
| 248 | 0.000255 | 0.011 | | | |
| 249 | 0.000023 | 0.007 | | | |
| 250 | 0.000076 | 0.030 | | | |
| 251 | 0.000066 | 0.083 | | | |
| 252a | 0.000341 | 0.069 | | | |
| 252b | 0.000250 | 0.315 | | | |
| 253a | 0.001474 | ~0.540 | | | |
| 253b | 0.001726 | >1 | | | |
| 254 | 0.000071 | 0.019 | | | |
| 255 | 0.000102 | 0.010 | | | |
| 256 | 0.000073 | 0.020 | | | |
| 257 | 0.000031 | 0.007 | | | |
| 258 | 0.000021 | 0.011 | | | |
| 259 | 0.000031 | 0.138 | | | |
| 260 | 0.000042 | ~0.0276 | | | |
| 261 | 0.000213 | 0.217 | | | |
| 262 | 0.000018 | 0.015 | 0.015 | | 2.9 |
| 263 | 0.000258 | 0.175 | | | |
| 264 | 0.000034 | 0.025 | 0.020 | | 13.5 |
| 265 | 0.000097 | 0.046 | | | |
| 266 | 0.000349 | 0.431 | | | |
| 267 | 0.00024 | 0.166 | | | |
| 268 | 0.000149 | 0.037 | 0.088 | | >15 |
| 269 | 0.001699 | 0.018 | | | |
| 270 | 0.000311 | 0.177 | | | |
| 271 | 0.000368 | 0.288 | | | |
| 272 | 0.000310 | ~0.27 | | | |
| 273 | 0.001080 | >1 | | | |
| 274 | 0.002422 | >1 | | | |
| 275 | 0.000945 | >1 | | | |
| 276 | 0.000532 | ~0.462 | | | |
| 277 | 0.000481 | ~0.502 | | | |
| 278 | 0.000323 | 0.342 | | | |
| 279 | 0.000025 | 0.017 | 0.012 | | 5.1 |
| 280 | 0.000077 | 0.051 | | | |
| 281 | 0.000058 | 0.013 | 0.010 | | 4.2 |
| 282 | 0.003272 | >1 | | | |
| 283 | 0.000610 | 0.440 | | | |
| 284 | 0.004185 | ~2.36 | | | |
| 285 | 0.001485 | ~1.33 | | | |
| 286 | 0.000046 | 0.059 | | | |
| 287 | 0.000034 | 0.026 | | | |
| 288 | 0.000059 | 0.043 | | | |
| 289 | 0.000055 | 0.031 | | | |
| 290 | 0.000393 | 0.170 | | | |
| 291 | 0.000594 | 0.468 | | | |
| 292a | 0.000132 | | | | |
| 292b | 0.000562 | ~0.586 | | | |
| 293a | 0.000459 | 0.148 | | | |
| 293b | 0.000152 | 0.259 | | | |
| 294a | 0.000237 | 0.365 | | | |
| 294b | 0.000077 | 0.153 | | | |
| 295 | 0.000171 | 0.338 | | | |
| 296 | 0.000339 | 0.549 | | | |
| 297 | 0.000477 | >1 | | | |
| 298 | 0.000083 | 0.178 | | | |
| 299 | 0.000151 | ~0.281 | | | |
| 300 | 0.000116 | 0.055 | | | |
| 301 | 0.000191 | 0.144 | | | |
| 302 | 0.000050 | 0.078 | | | |
| 303 | 0.000179 | ~0.077 | | | |
| 304 | 0.002803 | >1 | | | |
| 305 | 0.000146 | ~0.297 | | | |
| 306 | 0.000468 | 0.159 | | | |
| 307 | 0.000044 | 0.048 | | | |
| 308 | 0.060201 | >1 | | | |
| 309a | 0.001300 | | | | |
| 309b | 0.000111 | >1 | | | |
| 310 | 0.000119 | 0.351 | | | |
| 311 | 0.000031 | 0.018 | 0.017 | | 5.2 |
| 311a | 0.000037 | 0.014 | 0.018 | | 7.7 |
| 311b | 0.000077 | 0.026 | 0.035 | | >15 |
| 312 | 0.000058 | 0.055 | 0.033 | | >15 |
| 313 | 0.000155 | 0.552 | 0.179 | | |
| 313a | 0.000111 | ~0.299 | | | |
| 313b | 0.000713 | ~0.713 | | | |
| 314 | 0.000055 | 0.019 | 0.006 | | |
| 315 | 0.000071 | 0.037 | 0.010 | | 2.4 |
| 316 | 0.000055 | 0.029 | 0.012 | | 8.9 |
| 317 | 0.000291 | 0.065 | | | |
| 318 | 0.000220 | 1.377 | | | |
| 319 | 0.000293 | 0.240 | 0.139 | | >15 |
| 320 | 0.000078 | 0.023 | 0.019 | | 6.9 |
| 320a | 0.000189 | 0.024 | | | |
| 320b | 0.000104 | 0.062 | | | |
| 321 | 0.000257 | 0.413 | 0.232 | | |
| 322 | 0.000152 | ~0.547 | 0.323 | | |
| 323 | 0.000080 | ~0.146 | 0.142 | | |
| 324 | 0.000267 | 0.642 | 0.553 | | |
| 325 | 0.000337 | 1.336 | 1.049 | | |
| 326 | 0.000038 | 0.023 | 0.016 | | |
| 327 | 0.000819 | ~0.616 | | | |
| 328 | 0.000101 | ~0.154 | 0.050 | 0.052 | |
| 329 | 0.000152 | 0.013 | | | |
| 330 | 0.000130 | 0.010 | | | |
| 331 | 0.000117 | 0.029 | | | |
| 332 | 0.000130 | 0.196 | | | |
| 333 | 0.000251 | ~0.394 | | | |
| 334 | 0.000224 | 0.012 | | | |
| 335 | 0.000193 | 0.018 | | | |
| 336 | 0.000128 | 0.033 | | | |
| 337 | 0.000092 | 0.024 | | | |
| 338 | 0.000185 | 0.037 | | | |
| 339 | 0.000193 | 0.068 | | | |
| 340 | 0.000034 | >1 | | | |
| 341 | 0.000037 | 0.020 | | | |
| 342 | 0.000188 | 0.107 | | | |
| 343 | 0.000045 | 0.013 | | | |
| 344 | ~0.000058 | 0.014 | | | |
| 345 | 0.000204 | 0.036 | | | |
| 346 | 0.000116 | 0.011 | | | |
| 347 | 0.000045 | 0.144 | | | |
| 348 | 0.000059 | 0.033 | | | |
| 349 | 0.006685 | >1 | | | |
| 350 | 0.000583 | ~1.00 | | | |
| 351a | 0.000040 | 0.012 | | | |
| 351b | 0.000255 | 0.182 | | | |
| 352a | 0.000102 | 0.013 | 0.007 | 0.011 | >15 |
| 352b | 0.000247 | 0.266 | 0.140 | 0.263 | >15 |
| 353a | 0.000094 | 0.012 | 0.006 | 0.011 | >15 |
| 353b | 0.000238 | 0.139 | | 0.236 | |
| 354 | 0.000040 | 0.018 | | | |
| 357 | 0.000035 | 0.015 | 0.008 | | 3.0 |
| 358 | 0.000223 | 0.386 | | | |
| 359 | 0.000166 | 0.358 | 0.079 | | |
| 360 | 0.000127 | 0.107 | | | |
| 361 | 0.000149 | | | | |
| 362a | 0.000149 | 0.184 | | | |
| 362b | 0.000067 | 0.019 | 0.012 | | |
| 363a | 0.000228 | 0.359 | | | |
| 363b | 0.000093 | ~0.1745 | | | |
| 364a | 0.000098 | 0.225 | | | |
| 364b | 0.000292 | ~0.6699 | | | |
| 365a | 0.000102 | 0.035 | | | |
| 365b | 0.000081 | 0.011 | 0.008 | 0.011 | |
| 366 | 0.000067 | ~0.3194 | | | |
| 367 | 0.000201 | >1 | | | |
| 503 | 0.010457 | | | | |
| 504 | 0.023939 | >1 | | | |
| 505 | 0.003110 | | | | |
| 507 | 0.006605 | | | | |
| 508 | 0.003680 | | | | |
| 509 | 0.004525 | | | | |
| 510 | 0.002946 | | | | |
| 522 | 0.010590 | | | | |
| 523a | 0.000376 | >1 | | | |
| 523b | 0.000124 | 0.062 | | | |
| 524b | 0.008624 | 2.120 | | | |
| 524a | 0.019002 | 2.557 | | | |
| 528 | 0.000514 | | | | |
| 533 | 0.000808 | | | | |
| 534 | 0.000556 | | | | |

What is claimed is:

1. A compound of Formula (I) 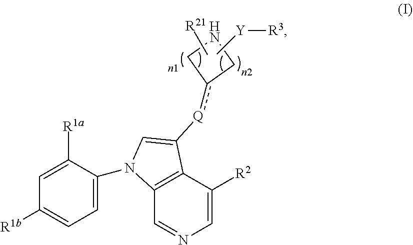
or a tautomer or a stereoisomeric form thereof, wherein
Q represents —CHR^{y}—, —O—, —C(═O)—, —NR^{q}—, or —CR^{y}═; the dotted line is an optional additional bond to form a double bond in case Q represents —CR^{y}═;
R^{1a} represents hydrogen; cyano; halo; Het; —C(═O)—NR^{xa}R^{xb}; —S(═O)₂—R¹⁸; —C(═O)—O—C₁₋₄alkyl-NR^{22a}R^{22b}; —C(═O)—O—C₁₋₄alkyl; 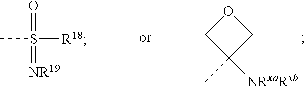
R¹⁸ represents C₁₋₆alkyl or C₃₋₆cycloalkyl;
R¹⁹ represents hydrogen or C₁₋₆alkyl;
or R¹⁸ and R¹⁹ are taken together to form —(CH₂)₃—, —(CH₂)₄— or —(CH₂)₅—;
Het represents a monocyclic 5- or 6-membered aromatic ring containing one, two or three O-, S- or N-atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, —C₁₋₄alkyl-OH, halo, CF₃, C₃₋₆cycloalkyl, Het³, and NR^{11c}R^{11d};
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents selected from the group consisting of halo and OR²³;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents each independently selected from the group consisting of halo and OR²³;
R²³ represents hydrogen or C₁₋₄alkyl optionally substituted with one, two or three halo;
R^{1b} represents hydrogen, F, Cl, or —O—C₁₋₄alkyl;
R² represents halo, C₃₋₆cycloalkyl, C₁₋₄alkyl, —O—C₁₋₄alkyl, cyano, or C₁₋₄alkyl substituted with one, two or three halo substituents;
R² represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond or 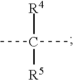
n1 is selected from 1 and 2;
n2 is selected from 1, 2, 3 and 4;
R^{y} represents hydrogen, —OH, C₁₋₄alkyl, —C₁₋₄alkyl-0H, or —C₁₋₄alkyl-O—C₁₋₄alkyl;
R^{q} represents hydrogen or C₁₋₄alkyl;
R⁵ represents hydrogen, C₁₋₄alkyl, or C₃₋₆cycloalkyl;
R³, R^{3a}, and R⁴ are each independently selected from the group consisting of Het¹; Het²;
Cy²; C₁₋₈alkyl; and C₁₋₈alkyl substituted with one, two, three or four substituents each independently selected from the group consisting of —C(═O)—NR^{10a}R^{10b}, —C(═O)—Het^{6a}, —C(═O)—Het^{6b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², Ar¹, and Cy²;
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo,
—OH, —O—C₁₋₄alkyl, —(C═O)—C₁₋₄alkyl, —S(═O)₂—C₁₋₄alkyl, and cyano;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;
R^{8a} and R^{8b} are each independently selected from the group consisting of hydrogen;
C₁₋₆alkyl; —(C═O)—C₁₋₄alkyl; and C₁₋₆alkyl substituted with one, two or three substituents each independently selected from the group consisting of —OH, cyano, halo, —S(═O)₂—C₁₋₄alkyl,
—O—C₁₋₄alkyl, —C(═O)—NR^{10a}R^{10b}, and —NR^{10c}—C(═O)—C₁₋₄alkyl;
Ar¹ represents phenyl optionally substituted with one, two or three substituents each independently selected from the group consisting of C₁₋₄alkyl, halo, —O—C₁₋₄alkyl, —CF₃, —OH,
—S(═O)₂—C₁₋₄alkyl, and —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a} Het^{6b}, C₁₋₄alkyl, oxo, —NR^{9a}R^{9b} and —OH—;
Het² represents C-linked pyrazolyl, 1,2,4-oxadiazolyl, pyridazinyl or triazolyl; which may be optionally substituted on one nitrogen atom with R^{6a};
R⁶ and R^{6a} are each independently selected from the group consisting of Het³; Het⁴; —C(═O)—NH—Cy¹; —C(═O)—NH—R⁸; —C(═O)—Het^{6a}; —C(═O)—NR^{10d}R^{10c}; —C(═O)—O—C₁₋₄alkyl; —S(═O)₂—C₁₋₄alkyl;
C₁₋₆alkyl optionally substituted with one or two substituents each independently selected from the group consisting of Het³, Het⁴, Het^{6a}, Hetb, Cy¹, —CN, —OH, —O—C₁₋₄alkyl, —C(═O)—NH—C₁₋₄alkyl, —C(═O)—N(C₁₋₄alkyl)₂, —C(═O)—NH—C₁₋₄alkyl-C₃₋₆cycloalkyl, —C(═O)—OH, —NR^{11a}R^{11b} and —NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —C(═O)—N(C₁₋₄alkyl)₂, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R⁸ represents hydrogen, —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b},
—S(═O)₂—C₁₋₄alkyl, Het^{3a}, and Het^{6a};
Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo,
—OH, —NR^{11a}R^{11b}, or oxo; and wherein said heterocyclyl is optionally substituted on one nitrogen atom with C₁₋₄alkyl or —(C═O)—C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₇₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het¹, and —C(═O)—NR^{10a}R^{10b};
Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-O-1, —O—C₁₋₄alkyl, —O—(C═O)—NR^{10a}R^{10b}, and
—O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};
Het^{6b} and Het^{8b} each independently represent a bicyclic N-linked 6- to 1l-membered fully or partially saturated heterocyclyl containing one N-atom and optionally one or two additional heteroatoms each independently selected from O, S, and N, wherein said S-atom might be substituted to form S(═O) or S(═O); wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl,
—NH—C(═O)—Cy³, and —O—C₁₋₄alkyl; and wherein said heterocyclyl is optionally substituted on one nitrogen with a substituent selected from the group consisting of —C(═O)—C₁₋₄alkyl,
—C(═O)—Cy³, —(C═O)—C₁₋₄alkyl-OH, —C(═O)—C₁₋₄alkyl-O—C₁₋₄alkyl,
—C(═O)—C₁₋₄alkyl-NR^{11a}R^{11b} and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl,
—NH—S(═O)₂—C₁₋₄alkyl, —S(═O)₂—C₁₋₄alkyl, and —O—C₁₋₄alkyl;
Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, —O—C₁₋₄alkyl, cyano, 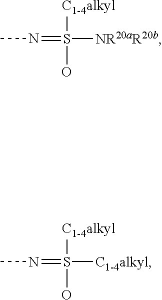
and
C₁₋₄alkyl substituted with one or two substituents each independently selected from the group consisting of Het^{3a}, Het^{6a}, Het^{6b}, and —NR^{9a}R^{9b},
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen;
C₁₋₄alkyl; C₃₋₆cycloalkyl; —C(═O)—C₁₋₄alkyl; —C(═O)—C₃₋₆cycloalkyl; —S(═O)₂—C₁₋₄alkyl;
Het⁵; Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, R^{17b}, R^{20a}, R^{20b}, R^{22a}, and R^{22b} are each independently selected from the group consisting of hydrogen and C₁₋₄alkyl;
R^{11c} and R^{11d} are each independently selected from the group consisting of hydrogen, C₁₋₆alkyl, and —C(═O)—C₁₋₄alkyl;
R^{10a}, R^{10b} and R^{10c} are each independently selected from the group consisting of hydrogen,
C₁₋₄alkyl, and C₃₋₆cycloalkyl;
R^{10d} and R^{10e} are each independently selected from the group consisting of C₁₋₄alkyl, —O—C₁₋₄alkyl and C₃₋₆cycloalkyl;
R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het⁵, Het¹, or Het⁸;
or a pharmaceutically acceptable salt or a solvate thereof.

2. The compound according to claim 1, wherein
Q represents —CHR^{y}—, —O—, —C(═O)—, —NR^{q}—, or —CR^{y}═; the dotted line is an optional additional bond to form a double bond in case Q represents —CR^{y}═;
R^{1a} represents hydrogen; cyano; halo; Het; —C(═O)—NR^{xa}R^{xb}; —S(═O)₂—R¹⁸; 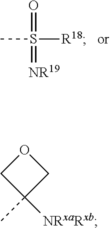
R¹⁸ represents C₁₋₆alkyl or C₃₋₆cycloalkyl;
R¹⁹ represents hydrogen or C₁₋₆alkyl;
Het represents a monocyclic 5- or 6-membered aromatic ring containing one, two or three nitrogen atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, or cyano;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and NR^{11c}R^{11d};
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, —C₁₋₄alkyl-O—C₁₋₄alkyl, and cyano;
R^{1b} represents hydrogen, F or Cl;
R² represents halo, C₃₋₆cycloalkyl, C₁₋₄alkyl, —O—C₁₋₄alkyl, cyano, or C₁₋₄alkyl substituted with one, two or three halo substituents;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond or 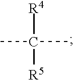
n1 and n2 are each independently selected from 1 and 2;
R^{y} represents hydrogen, —OH, C₁₋₄alkyl, —C₁₋₄alkyl-OH, or —C₁₋₄alkyl-O—C₁₋₄alkyl;
R^{q} represents hydrogen or C₁₋₄alkyl;
R⁵ represents hydrogen, C₁₋₄alkyl, or C₃₋₆cycloalkyl;
R³, R^{3a}, and R⁴ are each independently selected from the group consisting of Het¹; Het²; Cy²;
C₁₋₆alkyl; and C₁₋₆alkyl substituted with one, two, three or four substituents each independently selected from the group consisting of —C(═O)—NR^{10a}R^{10b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², and Cy²;
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano; or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo,
—OH, —O—C₁₋₄alkyl, —(C═O)—C₁₋₄alkyl, —S(═O)₂—C₁₋₄alkyl, and cyano;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl-S(═O)₂—C₁₋₄alkyl, and cyano;
R^{8a} and R^{8b} are each independently selected from the group consisting of hydrogen;
C₁₋₆alkyl; and C₁₋₆alkyl substituted with one, two or three substituents each independently selected from the group consisting of —OH, cyano, halo, —S(═O)₂—C₁₋₄alkyl, —O—C₁₋₄alkyl,
—C(═O)—NR^{10a}R^{10b}, and —NR^{10c}—C(═O)—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy¹, and
—C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl or triazolyl; which may be optionally substituted on one nitrogen atom with R^{6a};
R⁶ and R^{6a} are each independently selected from the group consisting of Het³; Het⁴; —C(═O)—NH—Cy¹; —C(═O)—NH—R^{B}; —S(═O)₂—C₁₋₄alkyl;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—NH—C₁₋₄alkyl-C₃₋₆cycloalkyl, —C(═O)—OH, —NR^{11a}R^{11b}, and
—NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R⁸ represents —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b}, Het^{3a}, and Het^{6a};
Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo,
—OH, —NR^{11a}R^{11b}, or oxo; and wherein said heterocyclyl is optionally substituted on one nitrogen atom with C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano,
—COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)₂—C₁₋₄alkyl, cyano, C₁₋₄alkyl, —C₁₋₄alkyl-OH, —O—C₁₋₄alkyl, —O—(C═O)—NR^{10a}R^{10b}, and
—O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};
Het^{6b} and Het^{8b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl,
—NH—C(═O)—Cy³, and —O—C₁₋₄alkyl; and wherein said heterocyclyl is optionally substituted on one nitrogen with a substituent selected from the group consisting of —C(═O)—C₁₋₄alkyl,
—C(═O)—Cy³, —(C═O)—C₁₋₄alkyl-OH, —C(═O)—C₁₋₄alkyl-O—C₁₋₄alkyl,
—C(═O)—C₁₋₄alkyl-NR^{11a}R^{11b}, and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl,
—NH—S(═O)₂—C₁₋₄alkyl, —S(═O)₂—C₁₋₄alkyl, and —O—C₁₋₄alkyl;
Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, 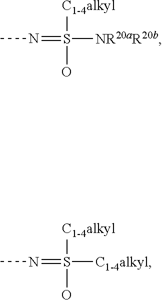
and
C₁₋₄alkyl substituted with one or two substituents each independently selected from the group consisting of Het^{3a}, Het^{6a}, Het^{6b}, and —NR^{9a}R^{9b};
Cy³ represents C₃₋₇Cycloalkyl; wherein said C₃₋₇cycloalkyl is optionally substituted with one, two or three halo substituents;
R^{9a} and R^{9b} are each independently selected from the group consisting of hydrogen;
C₁₋₄alkyl; C₃₋₆cycloalkyl; —C(═O)—C₁₋₄alkyl; —C(═O)—C₃₋₆cycloalkyl; —S(═O)₂—C₁₋₄alkyl; Het⁵; Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, R^{17b}, R^{20a}, and R^{20b} are each independently selected from the group consisting of hydrogen and C₁₋₄alkyl;
R^{11c} and R^{11d} are each independently selected from the group consisting of hydrogen, C₁₋₆alkyl, and —C(═O)—C₁₋₄alkyl;
R^{10a} and R^{10b} are each independently selected from the group consisting of hydrogen, C₁₋₄alkyl, and C₃₋₆cycloalkyl;
R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het, Het¹, or Het⁸;
or a pharmaceutically acceptable salt or a solvate thereof.

3. The compound according to claim 1, wherein
Q represents —CHR^{y}— or —CR^{y}═; the dotted line is an optional additional bond to form a double bond in case Q represents —CR^{y}═;
R^{1a} represents hydrogen; halo; —C(═O)—NR^{a}R^{xb}; —S(═O)₂—R¹⁸,
—C(═O)—O—C₁₋₄alkyl; or 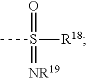
R¹⁸ represents C₁₋₆alkyl;
R¹⁹ represents hydrogen or C₁₋₆alkyl;
or R¹⁸ and R¹⁹ are taken together to form —(CH₂)₃—, —(CH₂)₄— or —(CH₂)₅—;
R^{xa} and R^{xb} are each independently selected from the group consisting of hydrogen;
Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, and —C₁₋₄alkyl-OH;
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, —O—C₁₋₄alkyl, and C₁₋₄alkyl substituted with one, two or three OR³;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three —OH substituents;
R²³ represents hydrogen or C₁₋₄alkyl;
R^{1b} represents F or —O—C₁₋₄alkyl;
R² represents halo, C₁₋₄alkyl, or C₁₋₄alkyl substituted with one, two or three halo substituents;
R²¹ represents hydrogen or —Y^{a}—R^{3a}; provided that when R²¹ represents —Y^{a}—R^{3a}, one of —Y^{a}—R^{3a} and —Y—R³ is attached to the nitrogen atom of the ring;
Y and Y^{a} each independently represent a covalent bond or 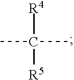
R⁵ represents hydrogen;
n1 is selected from 1 and 2;
n2 is selected from 1, 2 and 3;
R^{y} represents hydrogen;
R³, R^{3a}, and R⁴ are each independently selected from the group consisting of Het¹; C¹⁻⁸alkyl; and C₁₋₈alkyl substituted with one, two, three or four substituents each independently selected from the group consisting of —C(═O)—Het^{6a}, —C(═O)—Het^{6b}, —NR^{10c}—C(═O)—C₁₋₄alkyl, —NRR^{xc}R^{xd}, —NR^{8a}R^{8b}, —CF₃, halo, —OH, —O—C₁₋₄alkyl, Het¹, Het², Ar¹, and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of
—(C═O)—C₁₋₄alkyl, and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
R^{8a} and R^{8b} are each independently selected from the group consisting of hydrogen; C₁₋₆alkyl; —(C═O)—C₁₋₄alkyl; and C₁₋₆alkyl substituted with one, two or three —O—C₁₋₄alkyl;
Ar¹ represents phenyl optionally substituted with one, two or three substituents each independently selected from the group consisting of C₁₋₄alkyl and —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶, —C(═O)—Cy, and —C(═O)—R⁸; and wherein said heterocyclyl is optionally substituted on one or two carbon atoms with in total one, two, three or four substituents each independently selected from the group consisting of halo, R⁶, C₁₋₄alkyl, oxo and —OH;
Het² represents C-linked pyrazolyl, 1,2,4-oxadiazolyl, pyridazinyl or triazolyl;
R⁶ is selected from the group consisting of Het³; Het⁴; —C(═O)—NH—Cy¹; —C(═O)—NH—R; —C(═O)—Het^{6a}; —C(═O)—NR^{10d}R^{10e}; —C(═O)—O—C₁₋₄alkyl; —S(═O)₂—C₁₋₄alkyl;
C₁₋₆alkyl optionally substituted with one or two substituents each independently selected from the group consisting of Het^{6a}, Het^{6b}, and —OH;
R⁸ represents hydrogen, —O—C₁₋₆alkyl, C₁₋₆alkyl; or C₁₋₆alkyl substituted with one, two or three substituents each independently selected from —OH, —O—C₁₋₄alkyl, cyano, —S(═O)₂—C₁₋₄alkyl, and Het^{3a};
Het³ and Het^{3a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen atom with
—(C═O)—C₁₋₄alkyl;
Het⁴ represents a monocyclic C-linked 5- or 6-membered aromatic ring containing one, two or three heteroatoms each independently selected from O, S, and N; wherein said aromatic ring is optionally substituted on one or two carbon atoms with in total one or two substituents each independently selected from the group consisting of C₁₋₄alkyl and —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo and —S(═O)₂—C₁₋₄alkyl; and wherein said heterocyclyl is optionally substituted on one nitrogen with a substituent selected from the group consisting of —C(═O)—C₁₋₄alkyl and —S(═O)₂—C₁₋₄alkyl;
Het^{6b} represents a bicyclic N-linked 6- to 1l-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with a
—C(═O)—C₁₋₄alkyl;
Cy¹ represents C₃₋₆cycloalkyl optionally substituted with one, two or three —OH;
Cy² represents C₃₋₇cycloalkyl or a 5- to 12-membered saturated carbobicyclic system;
wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, and C₁₋₄alkyl;
R^{9a} and R^{9b} are each independently selected from the group consisting of hydrogen;
C₁₋₄alkyl; —C(═O)—C₁₋₄alkyl; —S(═O)₂—C₁₋₄alkyl; and —C(═O)—R¹⁴;
R^{10a}, R^{10b} and R^{10c} are each independently selected from the group consisting of hydrogen and
C₁₋₄alkyl;
R^{10d} and R^{10e} are each independently selected from the group consisting of C₁₋₄alkyl and —O—C₁₋₄alkyl;
R¹⁴ represents —O—C₁₋₄alkyl.

4. The compound according to claim 1, wherein
Q represents —CHR^{y}—, or —CR^{y}═; the dotted line is an optional additional bond to form a double bond in case Q represents —CR^{y}═;
R^{1a} represents hydrogen; halo; —C(═O)—NR^{xa}R^{xb}; or 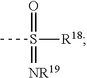
R^{xa} and R^{xb} are each independently selected from the group consisting of hydrogen;
Het³; and C₁₋₆alkyl; wherein optionally said C₁₋₆alkyl are substituted with 1, 2 or 3 substituents each independently selected from the group consisting of —OH, and —OC₁₋₄alkyl;
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, and —O—C₁₋₄alkyl;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, —OH, and —O—C₁₋₄alkyl;
R^{1b} represents F;
R² represents halo, C₁₋₄alkyl, or C₁₋₄alkyl substituted with one, two or three halo substituents;
R²¹ represents hydrogen;
R^{y} represents hydrogen;
R⁵ represents hydrogen;
R³ and R⁴ are each independently selected from the group consisting of Het¹; Cy²;
C₁₋₆alkyl; and C₁₋₆alkyl substituted with one, two, three or four substituents each independently selected from the group consisting of —NR^{xc}R^{xd}, —NR^{8a}R^{8b}, —CF₃, —OH, Het¹, and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of
—(C═O)—C₁₋₄alkyl and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
R^{8a} and R^{8b} are each independently selected from the group consisting of C₁₋₆alkyl; and C₁₋₆alkyl substituted with one —O—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R⁶ and —C(═O)—R⁸; and wherein said heterocyclyl is optionally substituted on one or two carbon atoms with in total one or two substituents each independently selected from the group consisting of oxo and —NR^{9a}R^{9b};
R⁶ represents Het⁴; —C(═O)—NH—R^{B}; —S(═O)₂—C₁₋₄alkyl; or C₁₋₆alkyl;
R⁸ represents —O—C₁₋₆alkyl, C₁₋₆alkyl; or C₁₋₆alkyl substituted with one, two or three substituents each independently selected from —O—C₁₋₄alkyl, and cyano;
Het³ represents a monocyclic C-linked 4- to 7-membered fully or partially saturated heterocyclyl containing one, two or three heteroatoms each independently selected from O, S, and N;
Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two —S(═O)₂—C₁₋₄alkyl; and wherein said heterocyclyl is optionally substituted on one nitrogen with a substituent selected from the group consisting of
—C(═O)—C₁₋₄alkyl and —S(═O)₂—C₁₋₄alkyl;
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of R⁶,
—C(═O)—Het^{6a}, Het^{6a}, Het^{6b}, —NR^{9a}R^{9b}, 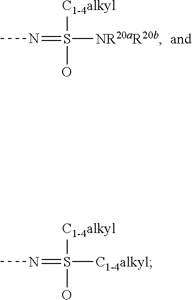
R^{9a} and R^{9b} are each independently selected from the group consisting of hydrogen; C₁₋₄alkyl; —C(═O)—C₁₋₄alkyl; and —S(═O)₂—C₁₋₄alkyl.

5. The compound according to claim 4, wherein
Q represents —CHR^{y}—, or —CR^{y}═; the dotted line is an optional additional bond to form a double bond in case Q represents —CR^{y}═;
R^{1a} represents hydrogen; halo; or —C(═O)—NR^{xa}R^{xb};
R^{xa} and R^{xb} are each independently selected from the group consisting of hydrogen and C₁₋₆alkyl;
R^{1b} represents F;
R² represents halo, C₁₋₄alkyl, or C₁₋₄alkyl substituted with one, two or three halo substituents;
R²¹ represents hydrogen;
R^{y} represents hydrogen;
R⁵ represents hydrogen;
R³ and R⁴ are each independently selected from the group consisting of Het¹; Cy²;
C₁₋₆alkyl; and C₁₋₆alkyl substituted with one, two, three or four substituents each independently selected from the group consisting of —NR^{xc}R^{xd}, —NR^{8a}R^{8b}, Het¹, and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of
—(C═O)—C₁₋₄alkyl and —S(═O)₂—C₁₋₄alkyl;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of —(C═O)—C₁₋₄alkyl and —S(═O)₂—C₁₋₄alkyl;
R^{8a} and R^{8b} are each independently selected from the group consisting of C₁₋₆alkyl; and C₁₋₆alkyl substituted with one —O—C₁₋₄alkyl;
Het¹ represents a monocyclic C-linked 4- to 7-membered fully or partially saturated heterocyclyl containing one, two or three heteroatoms each independently selected from O, S, and N; wherein said heterocyclyl is optionally substituted on one nitrogen with a substituent selected from the group consisting of R⁶ and —C(═O)—R⁸;
R⁶ represents Het⁴; —C(═O)—NH—R⁸; or —S(═O)₂—C₁₋₄alkyl;
R⁸ represents —O—C₁₋₆alkyl, C₁₋₆alkyl; or C₁₋₆alkyl substituted with one, two or three substituents each independently selected from —O—C₁₋₄alkyl, and cyano;
Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two —S(═O)₂—C₁₋₄alkyl; and wherein said heterocyclyl is optionally substituted on one nitrogen with a substituent selected from the group consisting of
—C(═O)—C₁₋₄alkyl and —S(═O)₂—C₁₋₄alkyl;
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with
—C(═O)—C₁₋₄alkyl;
Cy² represents C₃₋₇cycloalkyl optionally substituted with one, two, three or four substituents each independently selected from the group consisting of R⁶, Het^{6a}, Het^{6b}, and —NR^{9a}R^{9b};
R^{9a} and R^{9b} are each independently selected from the group consisting of hydrogen;
C₁₋₄alkyl; —C(═O)—C₁₋₄alkyl; and —S(═O)₂—C₁₋₄alkyl;
R^{10a} and R^{10b} are each independently selected from the group consisting of hydrogen and C₁₋₄alkyl.

6. The compound according to claim 5, wherein
Q represents —CHR^{y}—;
R^{1a} represents —C(═O)—NR^{xa}R^{xb};
R^{xa} and Rx represent C₁₋₆alkyl;
R^{1b} represents F;
R² represents halo or C₁₋₄alkyl;
R²¹ represents hydrogen;
R^{y} represents hydrogen;
R⁵ represents hydrogen;
R³ is selected from the group consisting of Het¹; Cy²; C₁₋₆alkyl; and C₁₋₆alkyl substituted with one, two, three or four substituents each independently selected from the group consisting of —NR^{xc}R^{xd}, Het¹, and Cy²;
R⁴ represents C₁₋₆alkyl; in particular isopropyl;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;
Het¹ represents a monocyclic C-linked 4- to 7-membered fully or partially saturated heterocyclyl containing one, two or three heteroatoms each independently selected from O, S, and N; wherein said heterocyclyl is optionally substituted on one nitrogen with a substituent selected from the group consisting of R⁶ and —C(═O)—R⁸;
R⁶ represents Het⁴ or —C(═O)—NH—R^{B};
R⁸ represents C₁₋₆alkyl; or C₁₋₆alkyl substituted with one, two or three substituents each independently selected from —O—C₁₋₄alkyl, and cyano;
Het⁴ represents a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one or two carbon atoms with in total one or two —C(═O)—NR^{10a}R^{10b};
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with
—C(═O)—C₁₋₄alkyl;
Het^{6b} represents a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with —C(═O)—C₁₋₄alkyl;
Cy² represents C₃₋₇cycloalkyl optionally substituted with one, two, three or four substituents each independently selected from the group consisting of R⁶, Het^{6a}, Het^{6b}, and —NR^{9a}R^{9b};
R^{9a} and R^{9b} are each independently selected from the group consisting of hydrogen; and —S(═O)₂—C₁₋₄alkyl;
R^{10a} and R^{10b} are each independently selected from the group consisting of hydrogen and C₁₋₄alkyl.

7. The compound according to claim 6, wherein
Q represents —CHR^{y}—;
R^{1a} represents —C(═O)—NR^{xa}R^{xb};
R^{xa} and R^{xb} represent C₁₋₆alkyl;
R^{1b} represents F;
R² represents C₁₋₄alkyl;
R²¹ represents hydrogen;
R^{y} represents hydrogen;
R⁵ represents hydrogen;
R³ is selected from the group consisting of Cy²; and C₁₋₆alkyl substituted with one, two, three or four substituents each independently selected from the group consisting of —NR^{xc}R^{xd}, Het¹, and Cy²;
R⁴ represents C₁₋₆alkyl; in particular isopropyl;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N; wherein said heterocyclyl is optionally substituted with one, two or three —(C═O)—C₁₋₄alkyl;
Het¹ represents a monocyclic C-linked 4- to 7-membered fully or partially saturated heterocyclyl containing one, two or three heteroatoms each independently selected from O, S, and N; wherein said heterocyclyl is optionally substituted on one nitrogen with —C(═O)—R⁸;
R⁶ represents —C(═O)—NH—R^{B};
R⁸ represents C₁₋₆alkyl;
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂ wherein said heterocyclyl is optionally substituted on one nitrogen with
—C(═O)—C₁₋₄alkyl;
Cy² represents C₃₋₇cycloalkyl optionally substituted with one, two, three or four substituents each independently selected from the group consisting of R⁶ and Het^{6a}.

8. The compound according to claim 1, wherein
Q represents —CHR^{y}—;
R^{1a} represents —C(═O)—NR^{xa}R^{xb};
R^{xa} and R^{xb} are C₁₋₆alkyl optionally substituted with 1, 2 or 3 —OH;
R^{1b} represents F;
R² represents methyl;
R²¹ represents hydrogen or methyl;
Y represents a covalent bond;
n1 is 1;
n2 is selected from 1 and 2;
R^{y} represents hydrogen;
R³ is selected from C₁₋₈alkyl substituted with one, two, three or four substituents each independently selected from the group consisting of —NR^{xc}R^{xd}, Het¹ and Cy²;
R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three —(C═O)—C₁₋₄alkyl;
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with —C(═O)—R⁸; and wherein said heterocyclyl is optionally substituted on one carbon atom with oxo;
R⁸ represents C₁₋₆alkyl; or C₁₋₆alkyl substituted with one, two or three substituents each independently selected from —OH, —O—C₁₋₄alkyl and cyano;
Het^{6a} represents a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one nitrogen with
—C(═O)—C₁₋₄alkyl;
Cy² represents C₃₋₇cycloalkyl optionally substituted with one Het^{6a}.

9. The compound according to claim 1, wherein R²¹ represents hydrogen.

10. The compound according to claim 1 wherein R² represents methyl.

11. The compound according to claim 1, wherein R^{1b} represents F.

12. The compound according to claim 1, wherein —Y—R³ is attached to the nitrogen atom of the ring.

13. The compound according to claim 1, wherein Formula (I) is limited to Formula (I-x):

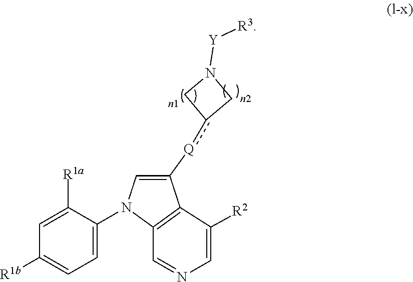

14. A compound of Formula (A) 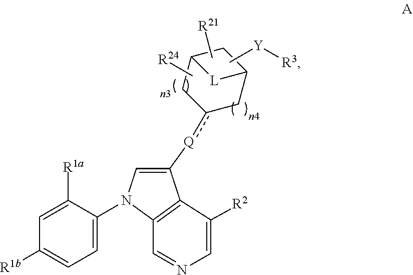
or a tautomer or a stereoisomeric form thereof, wherein
L is absent or represents —CH₂— or —CH₂—CH₂—;
Q represents —CHR^{y}—, —O—, —C(═O)—, —NR—, or —CR^{y}═; the dotted line is an optional additional bond to form a double bond in case Q represents —CR^{y}═;
R^{1a} represents hydrogen; cyano; halo; Het; —C(═O)—NR^{xa}R^{xb}; —S(═O)₂—R¹;
—C(═O)—O—C₁₋₄alkyl-NR^{22a}R^{22b}; —C(═O)—O—C₁₋₄alkyl; 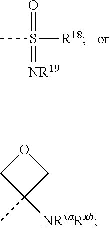
R¹⁸ represents C₁₋₆alkyl or C₃₋₆cycloalkyl;
R¹⁹ represents hydrogen or C₁₋₆alkyl;
or R¹⁸ and R¹⁹ are taken together to form —(CH₂)₃—, —(CH₂)₄— or —(CH₂)₅—;
Het represents a monocyclic 5- or 6-membered aromatic ring containing one, two or three O-, S- or N-atoms and optionally a carbonyl moiety; wherein said monocyclic 5- or 6-membered aromatic ring is optionally substituted with one, two or three substituents selected from the group consisting of C₁₋₄alkyl, C₃₋₆cycloalkyl, halo or cyano;
R^{xa} and R^{xb} are each independently selected from the group consisting of hydrogen; Het³; C₃₋₆cycloalkyl; and C₁₋₆alkyl; wherein optionally said C₃₋₆cycloalkyl and C₁₋₆alkyl are substituted with 1, 2 or 3 substituents each independently selected from the group consisting of —OH, —OC₁₋₄alkyl, —C₁₋₄alkyl-OH, halo, CF₃, C₃₋₆cycloalkyl, Het³, and NR^{11c}R^{11d};
or R^{xa} and R^{xb} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents selected from the group consisting of halo and OR²³;
or R^{xa} and R^{xb} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of C₁₋₄alkyl, halo, —OH, —O—C₁₋₄alkyl, cyano, and C₁₋₄alkyl substituted with one, two or three substituents each independently selected from the group consisting of halo and OR²³;
R²³ represents hydrogen or C₁₋₄alkyl optionally substituted with one, two or three halo;
R^{1b} represents hydrogen, F, Cl, or —O—C₁₋₄alkyl;
R² represents halo, C₃₋₆cycloalkyl, C₁₋₄alkyl, —O—C₁₋₄alkyl, cyano, or C₁₋₄alkyl substituted with one, two or three halo substituents;
R^{2a} represents hydrogen or C₁₋₄alkyl;
R²¹ represents hydrogen or —Y^{a}—R^{3a}; provided that when R^{2′} represents —Y^{a}—R^{3a}, one of —Y^{a}—R^{3a} and —Y—R³ is attached to the nitrogen atom of the ring;
Y and Y^{a} each independently represent a covalent bond or 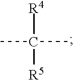
n3 is selected from 0 and 1;
n4 is selected from 0, 1, 2 and 3;
R^{y} represents hydrogen, —OH, C₁₋₄alkyl, —C₁₋₄alkyl-OH, or —C₁₋₄alkyl-O—C₁₋₄alkyl;
R^{q} represents hydrogen or C₁₋₄alkyl;
R⁵ represents hydrogen, C₁₋₄alkyl, or C₃₋₆cycloalkyl;
R³, R^{3a}, and R⁴ are each independently selected from the group consisting of Het¹; Het²;
Cy²; C₁₋₈alkyl; and C₁₋₈alkyl substituted with one, two, three or four substituents each independently selected from the group consisting of —C(═O)—NR^{10a}R^{10b}, —C(═O)—Het^{6a}, —C(═O)—Het^{6b}, —NR^{10c}—C(═O)—C₁₋₄alkyl, —S(═O)₂—C₁₋₄alkyl, —NR^{xc}R^{xd}, —NR^{8a}R^{8b}, —CF₃, cyano, halo, —OH, —O—C₁₋₄alkyl, Het¹, Het², Ar¹, and Cy²;
R^{xc} represents Cy¹; Het⁵; —C₁₋₆alkyl-Cy¹; —C₁₋₆alkyl-Het³; —C₁₋₆alkyl-Het⁴;
or —C₁₋₆alkyl-phenyl;
R^{xd} represents hydrogen; C₁₋₄alkyl; or C₁₋₄alkyl substituted with one, two or three substituents selected from the group consisting of halo, —OH, —O—C₁₋₄alkyl, and cyano;
or R^{xc} and R^{xd} are taken together to form together with the N-atom to which they are attached a 4- to 7-membered monocyclic fully or partially saturated heterocyclyl containing one N-atom and optionally one additional heteroatom selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl, —S(═O)₂—C₁₋₄alkyl, and cyano;
or R^{xc} and R^{xd} are taken together to form together with the N-atom to which they are attached a 6- to 11-membered bicyclic fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted with one, two or three substituents selected from the group consisting of halo, —OH, —O—C₁₋₄alkyl, —(C═O)—C₁₋₄alkyl —S(═O)₂—C₁₋₄alkyl, and cyano;
R^{8a} and R^{8b} are each independently selected from the group consisting of hydrogen;
C₁₋₆alkyl; —(C═O)—C₁₋₄alkyl; and C₁₋₆alkyl substituted with one, two or three substituents each independently selected from the group consisting of —OH, cyano, halo, —S(═O)₂—C₁₋₄alkyl, —O—C₁₋₄alkyl,
—C(═O)—NR^{10a}R^{10b}, and —NR^{10c}—C(═O)—C₁₋₄alkyl;
Ar¹ represents phenyl optionally substituted with one, two or three substituents each independently selected from the group consisting of C₁₋₄alkyl, halo, —O—C₁₋₄alkyl, —CF₃, —OH,
—S(═O)₂—C₁₋₄alkyl, and —C(═O)—NR^{10a}R^{10b};
Het¹ represents a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂; wherein said heterocyclyl is optionally substituted on one nitrogen with a substituent selected from the group consisting of R, —C(═O)—Cy¹, and —C(═O)—R⁸; and wherein said heterocyclyl is optionally substituted on one or two carbon atoms with in total one, two, three or four substituents each independently selected from the group consisting of halo, R⁶, Het^{6a}, Het^{6b}, C₁₋₄alkyl, oxo, —NR^{9a}R^{9b} and —OH;
Het² represents C-linked pyrazolyl, 1,2,4-oxadiazolyl, pyridazinyl or triazolyl; which may be optionally substituted on one nitrogen atom with R^{6a};
R⁶ and R^{6a} are each independently selected from the group consisting of Het³; Het⁴; —C(═O)—NH—Cy¹; —C(═O)—NH—R⁸; —C(═O)—Het^{6a}; —C(═O)—NR^{10d}R^{10e}; —C(═O)—O—C₁₋₄alkyl; —S(═O)₂—C₁₋₄alkyl;
C₁₋₆alkyl optionally substituted with one or two substituents each independently selected from the group consisting of Het³, Het⁴, Het^{6a}, Het^{6b}, Cy¹, —CN, —OH, —O—C₁₋₄alkyl, —C(═O)—NH—C₁₋₄alkyl, —C(═O)—N(C₁₋₄alkyl)₂, —C(═O)—NH—C₁₋₄alkyl-C₃₋₆cycloalkyl, —C(═O)—OH, —NR^{11a}R^{11b}, and
—NH—S(═O)₂—C₁₋₄alkyl; and
C₃₋₆cycloalkyl optionally substituted by one or two substituents each independently selected from the group consisting of —CN, —OH, —O—C₁₋₄alkyl, —C(═O)—NH—C₁₋₄alkyl, —C(═O)—N(C₁₋₄alkyl)₂, —NH—S(═O)₂—C₁₋₄alkyl, and C₁₋₄alkyl optionally substituted with one substituent selected from the group consisting of OH, —O—C₁₋₄alkyl, —C(═O)—NH—C₁₋₄alkyl and —NH—S(═O)₂—C₁₋₄alkyl;
R⁸ represents hydrogen, —O—C₁₋₆alkyl, C₁₋₆alkyl; or C₁₋₆alkyl substituted with one, two or three substituents each independently selected from —OH, —O—C₁₋₄alkyl, halo, cyano, —NR^{11a}R^{11b},
—S(═O)₂—C₁₋₄alkyl, Het^{3a}, and Het^{6a};
Het³, Het^{3a}, Het⁵ and Het^{5a} each independently represent a monocyclic C-linked 4- to 7-membered fully or partially saturated heterocyclyl containing one, two or three heteroatoms each independently selected from O, S, and N, wherein said S-atom might be substituted to form S(═O) or S(═O)₂; or a bicyclic C-linked 6- to 11-membered fully or partially saturated heterocyclyl containing one, two or three heteroatoms each independently selected from O, S, and N, wherein said S-atom might be substituted to form S(═O) or S(═O)₂;
wherein said heterocyclyl is optionally substituted on one carbon atom with C₁₋₄alkyl, halo, —OH, —NR^{11a}R^{11b}, or oxo; and wherein said heterocyclyl is optionally substituted on one nitrogen atom with C₁₋₄alkyl or —(C═O)—C₁₋₄alkyl;
Het⁴ and Het⁷ each independently represent a monocyclic C-linked 5- or 6-membered aromatic ring containing one, two or three heteroatoms each independently selected from O, S, and N, or a fused bicyclic C-linked 9- or 10-membered aromatic ring containing one, two, three or four heteroatoms each independently selected from O, S, and N; wherein said aromatic ring is optionally substituted on one nitrogen atom with C₁₋₄alkyl or —(C═O)—O—C₁₋₄alkyl; and wherein said aromatic ring is optionally substituted on one or two carbon atoms with in total one or two substituents each independently selected from the group consisting of —OH, halo, C₁₋₄alkyl, —O—C₁₋₄alkyl, —NR^{11a}R^{11b}, C₁₋₄alkyl-NR^{11a}R^{11b}, —NH—C(═O)—C₁₋₄alkyl, cyano, —COOH, —NH—C(═O)—O—C₁₋₄alkyl, —NH—C(═O)—Cy³, —NH—C(═O)—NR^{10a}R^{10b}, —(C═O)—O—C₁₋₄alkyl, —NH—S(═O)₂—C₁₋₄alkyl, Het^{8a}, —C₁₋₄alkyl-Het^{8a}, Het^{8b}, Het⁹, and —C(═O)—NR^{10a}R^{10b};
Het^{6a}, Het⁸ and Het^{8a} each independently represent a monocyclic N-linked 4- to 7-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one, two, three or four substituents each independently selected from the group consisting of halo, —OH, oxo,
—NH—C(═O)—C₁₋₄alkyl, —NH—C(═O)—Cy³, —(C═O)—NR^{10a}R^{10b}, —O—C₃₋₆cycloalkyl, —S(═O)—C₁₋₄alkyl, cyano, C₁₋₄alkyl, —C₁₋₄alkyl-OH, —O—C₁₋₄alkyl, —O—(C═O)—NR^{10a}R^{10b}and —O—(C═O)—C₁₋₄alkyl; and wherein said heterocyclyl is optionally substituted on one nitrogen with a substituent selected from the group consisting of —C(═O)—C₁₋₄alkyl, —S(═O)₂—C₁₋₄alkyl, and —(C═O)—NR^{10a}R^{10b};
Het^{6b} and Het^{6b} each independently represent a bicyclic N-linked 6- to 11-membered fully or partially saturated heterocyclyl containing one N-atom and optionally one or two additional heteroatoms each independently selected from O, S, and N, wherein said S-atom might be substituted to form S(═O) or S(═O)₂; wherein said heterocyclyl is optionally substituted on one or two carbon atoms with in total one or two substituents each independently selected from the group consisting of C₁₋₄alkyl, —OH, oxo, —(C═O)—NR^{10a}R^{10b}, —NH—C(═O)—C₁₋₄alkyl, —NH—C(═O)—Cy³, and —O—C₁₋₄alkyl; and wherein said heterocyclyl is optionally substituted on one nitrogen with a substituent selected from the group consisting of —C(═O)—C₁₋₄alkyl, —C(═O)—Cy³, —(C═O)—C₁₋₄alkyl-OH, —C(═O)—C₁₋₄alkyl-O—C₁₋₄alkyl,
—C(═O)—C₁₋₄alkyl-NR^{11a}R^{11b} and C₁₋₄alkyl;
Het⁹ represents a monocyclic C-linked 5- or 6-membered aromatic ring containing one, two or three heteroatoms each independently selected from O, S, and N, or a fused bicyclic C-linked 9- or 10-membered aromatic ring containing one, two or three heteroatoms each independently selected from O, S, and N; wherein said aromatic ring is optionally substituted on one nitrogen atom with C₁₋₄alkyl; and wherein said aromatic ring is optionally substituted on one or two carbon atoms with in total one or two substituents each independently selected from the group consisting of —OH, halo, and C₁₋₄alkyl;
Cy¹ represents C₃₋₆cycloalkyl optionally substituted with one, two or three substituents selected from the group consisting of —OH, —NH—C(═O)—C₁₋₄alkyl, C₁₋₄alkyl, —NH—S(═O)₂—C₁₋₄alkyl, —S(═O)₂—C₁₋₄alkyl, and —O—C₁₋₄alkyl;
Cy² represents C₃₋₇cycloalkyl or a 5- to 12-membered saturated carbobicyclic system; wherein said C₃₋₇cycloalkyl or said carbobicyclic system is optionally substituted with one, two, three or four substituents each independently selected from the group consisting of halo, R⁶, —C(═O)—Het^{6a}, Het^{6a}, Het^{6b}, —NR^{9a}R^{9b}, —OH, C₁₋₄alkyl, —O—C₁₋₄alkyl, cyano, 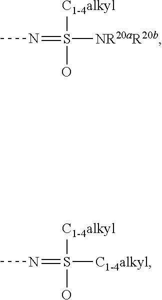
and
C₁₋₄alkyl substituted with one or two substituents each independently selected from the group consisting of Het^{3a}, Het^{6a}, Het^{6b}, and —NR^{9a}R^{9b};
Cy³ represents C₃₋₇cycloalkyl; wherein said C₃₋₇cycloalkyl is optionally substituted with one, two or three halo substituents;
R^{9a} and R^{9b} are each independently selected from the group consisting of hydrogen;
C₁₋₄alkyl; C₃₋₆cycloalkyl; —C(═O)—C₁₋₄alkyl; —C(═O)—C₃₋₆cycloalkyl; —S(═O)₂—C₁₋₄alkyl;
Het⁵; Het⁷; —C₁₋₄alkyl-R¹⁶; —C(═O)—C₁₋₄alkyl-Het^{3a}; —C(═O)—R¹⁴;
C₃₋₆cycloalkyl substituted with one, two or three substituents selected from the group consisting of halo, —OH, —O—C₁₋₄alkyl, —NR^{11a}R^{11b}, and cyano; and
C₁₋₄alkyl substituted with one, two or three substituents selected from the group consisting of halo, —OH, —O—C₁₋₄alkyl, —NR^{11a}R^{11b}, and cyano;
R^{11a}, R^{11b}, R^{13a}, R^{13b}, R^{15a}, R^{15b}, R^{17a}, R^{17b}, R^{20a}, R^{20b}, R^{22a}, and R^{22b} are each independently selected from the group consisting of hydrogen and C₁₋₄alkyl;
R^{11c} and R^{11d} are each independently selected from the group consisting of hydrogen, C₁₋₆alkyl, and —C(═O)—C₁₋₄alkyl;
R^{10a}, R^{10b} and R^{10c} are each independently selected from the group consisting of hydrogen,
C₁₋₄alkyl, and C₃₋₆cycloalkyl;
R^{10d} and R^{10e} are each independently selected from the group consisting of C₁₋₄alkyl, —O—C₁₋₄alkyl and C₃₋₆cycloalkyl;
R¹⁴ represents Het^{5a}; Het⁷; Het^{8a}; —O—C₁₋₄alkyl; —C(═O)NR^{15a}R^{15b}; C₃₋₆cycloalkyl substituted with one, two or three substituents selected from the group consisting of —O—C₁₋₄alkyl and halo; or C₁₋₄alkyl substituted with one, two or three substituents selected from the group consisting of —O—C₁₋₄alkyl, —NR^{13a}R^{13b}, halo, cyano, —OH, Het^{8a}, and Cy¹;
R¹⁶ represents —C(═O)—NR^{17a}R^{17b}, —S(═O)₂—C₁₋₄alkyl, Het⁵, Het⁷, or Het⁸;
R²⁴ represents hydrogen or C₁₋₄alkyl;
or a pharmaceutically acceptable salt or a solvate thereof.

15. A pharmaceutical composition comprising a compound as claimed in claim 1, or a pharmaceutically acceptable salt or a solvate thereof and a pharmaceutically acceptable carrier or excipient.

16. (canceled)

17. (canceled)

18. (canceled)

19. (canceled)

20. (canceled)

21. (canceled)

22. A method of treating or preventing cancer in a subject, comprising administering to a subject in need thereof, a therapeutically effective amount of a compound as claimed in claim 1 or a pharmaceutically acceptable salt or a solvate thereof.

23. The method of claim 22 wherein the cancer is selected from leukemias, lymphomas, myelomas or solid tumor cancers such as prostate cancer, lung cancer, breast cancer, pancreatic cancer, colon cancer, liver cancer, melanoma and glioblastoma.

24. A method of treating or preventing leukemia, myelodysplastic syndrome (MDS), and myeloproliferative neoplasms (MPN) in a subject, comprising administering to a subject in need thereof, a therapeutically effective amount of a compound of claim 1 or a pharmaceutically acceptable salt or a solvate thereof.

25. The method of claim 24 wherein the leukemia is selected from acute leukemias, chronic leukemias, myeloid leukemias, myelogeneous leukemias, lymphoblastic leukemias, lymphocytic leukemias, Acute myelogeneous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, and leukemias exhibiting HOX/MEIS1 gene expression signatures.

26. The method of claim 24 wherein the leukemia is (NPM1)-mutated leukemia.


SEQUENCE LISTING (1 sequence)

SEQ ID NO: 1 - protein, 616 aa, Artificial Sequence
  MENIN protein sequence with His tag
MGLKAAQKTLFPLRSIDDVVRLFAAELGREEPDLVLLSLVLGFVEHFLAVNRVIPTNVPELTFQPSPAPDPPGGLTYFPVADLSIIAALYARFTAQIRGAVDLSLYPREGGVSSRELVKKVSDVIWNSLSRSYFKDRAHIQSLFSFITGTKLDSSGVAFAVVGACQALGLRDVHLALSEDHAWVVFGPNGEQTAEVTWHGKGNEDRRGQTVNAGVAERSWLYLKGSYMRCDRKMEVAFMVCAINPSIDLHTDSLELLQLQQKLLWLLYDLGHLERYPMALGNLADLEELEPTPGRPDPLTLYHKGIASAKTYYRDEHIYPYMYLAGYHCRNRNVREALQAWADTATVIQDYNYCREDEEIYKEFFEVANDVIPNLLKEAASLLEAGEERPGEQSQGTQSQGSALQDPECFAHLLRFYDGICKWEEGSPTPVLHVGWATFLVQSLGRFEGQVRQKVRIVSREAEAAEAEEPWGEEAREGRRRGPRRESKPEEPPPPKKPALDKGLGTGQGAVSGPPRKPPGTVAGTARGPEGGSTAQVPAPAASPPPEGPVLTFQSEKMKGMKELLVATKINSSAIKLQLTAQSQVQMKKQKVSTPSDYTLSFLKRQRKGLHHHHHH